(19) United States
(12) Patent Application Publication
(10) Pub. No.: US 2025/0230470 A1
(43) Pub. Date: Jul. 17, 2025

(54) SAFETY SWITCHES TO CONTROL IN VITRO AND IN VIVO PROLIFERATION OF CELL THERAPY PRODUCTS

(71) Applicant: Sana Biotechnology, Inc., Seattle, WA (US)

(72) Inventor: Eleonore THAM, Seattle, WA (US)

(21) Appl. No.: 19/017,410

(22) Filed: Jan. 10, 2025

Related U.S. Application Data
(60) Provisional application No. 63/624,262, filed on Jan. 23, 2024.
(60) Provisional application No. 63/620,745, filed on Jan. 12, 2024.

(51) Int. Cl. C12N 15/90; C12N 5/00; C12N 5/071; C12N 9/64; C12N 9/78; C12N 15/86

(52) U.S. Cl. CPC C12N 15/907; C12N 5/0081; C12N 5/0678; C12N 9/6472; C12N 9/78; C12N 15/86; C12Y 304/22062; C12Y 305/04001; C12N 2506/45; C12N 2750/14143; C12N 2840/002

(57) ABSTRACT

The present disclosure provides modified cells comprising a nucleic acid encoding an agent (e.g., a detection agent, a selection agent, or a detection agent and a selection agent) inserted at an endogenous proliferation gene or off-target cell marker gene, as well as compositions, methods, uses, and kits related thereto.

### CROSS REFERENCE TO RELATED APPLICATIONS

This patent application claims priority benefits of U.S. Provisional Application No. 63/620,745 filed on Jan. 12, 2024 and U.S. Provisional Application No. 63/624,262 filed on Jan. 23, 2024, the content of each of which is incorporated herein by reference in their entirety.

### REFERENCE TO AN ELECTRONIC SEQUENCE LISTING

The contents of the electronic sequence listing (186152007700SEQLIST.xml.xml; Size: 154,521 bytes; and Date of Creation: Jan. 8, 2025) is herein incorporated by reference in its entirety.

### FIELD OF THE INVENTION

Provided herein are modified cells comprising a nucleic acid encoding an agent (e.g., a detection agent or selection agent) inserted at an endogenous proliferation gene or off-target cell marker gene, as well as compositions, methods, uses, and kits related thereto.

### BACKGROUND OF THE INVENTION

The ability to prevent or mitigate an adverse clinical event in a reliable and safe manner is key for successful development of stem cell-derived therapies. One challenge to stem cell-derived therapies is the growth of cancerous or other unwanted cells. Accordingly, there is an unmet need for developing safe and efficient methods for preserving differentiated therapeutic cells.

### BRIEF SUMMARY OF THE INVENTION

The present invention in one aspect provides a cell comprising a nucleic acid encoding a selection agent or a detection agent integrated at an endogenous proliferation gene locus and operably linked to the promoter of the endogenous proliferation gene, wherein expression of the nucleic acid encoding the selection agent or the detection agent is regulated by the promoter of the endogenous proliferation gene and/or regulatory elements of the endogenous proliferation gene and methods of uses thereof. The present invention in another aspect provides a cell comprising a nucleic acid encoding a selection agent or a detection agent integrated at an off-target cell type gene locus and operably linked to the promoter of the off-target cell marker gene, wherein expression of the nucleic acid encoding the selection agent or the detection agent is regulated by the promoter of the off-target cell marker gene and/or regulatory elements of the off-target cell marker gene and methods of uses thereof.

### BRIEF DESCRIPTION OF THE DRAWINGS

The drawings illustrate certain features and advantages of this disclosure. These embodiments are not intended to limit the scope of the appended claims in any manner.

FIG. 1 shows a schematic representation of pluripotent stem cell (PSC) and the subsequent implantation of differentiated cells into a human. Differentiated cells are a mixed pool, with most PSCs progressing to the post-mitotic stage during differentiation, while a small subset of PSCs and other cells with proliferative potential remain. These residual proliferating cells may cause tumors or teratomas that could evade the immune system upon transplantation.

FIG. 2 depicts a workflow for identification of endogenous proliferation gene candidates in iPSCs by bulk RNA sequencing, cross-referencing candidates with cancer-associated genes from internal and public datasets, and single cell RNA (scRNA) sequencing to interrogate proliferative gene specificity across cell types (e.g., beta islet cells and cardiomyocytes).

FIG. 3A depicts a heat map of bulk RNA expression of endogenous proliferation genes in sorted cadaveric islets (n=7 adult cadaveric beta cell batches; n=6 adult cadaveric alpha cell batches) and the average transcripts per million (TPM) calculated for each gene (data unscaled). Candidate genes demonstrated the largest fold change in expression among the datasets and had higher absolute TPM values sorted under the three batches of representative iPSC than the adult cadaveric beta and alpha cell batches. The genes from top to bottom are BIRC5, CDK1, TOP2A, PTTG1, CCNB1, TPX2, KIF11, SPC25, RRM2, CENPK, SMC4, TYMS, CDC20, AURKB, H2AZ1, TMSB15A, CENPF, and MKI67. FIG. 3B depicts a heatmap of bulk RNA expression of endogenous proliferation genes in sorted PSC cell lines (n=28) and differentiated batches (n=24) and the average TPM calculated for each gene (data unscaled). Fold change in expression of a gene was calculated as the average TPM of a gene in PSC batches divided by the average TPM of the same gene in differentiated batches. The genes from top to bottom are BIRC5, CDK1, TOP2A, PTTG1, CCNB1, TPX2, KIF11, CENPK, SMC4, TYSM, CDC20, AURKB, CENPF, and MKI67. FIG. 3C depicts a single-cell RNA (scRNA) sequencing map of two different PSC-derived cell types (n=22). The cells from scRNA sequencing were annotated based on their gene expression profiles to identify the proliferative subset of cells, as outlined by a circular shape in the figure. The proliferative subset of cells was then analyzed to identify endogenous proliferation genes specifically expressed in the proliferative subset and not in terminally differentiated cells, agnostic of cell type. The maps on the right depict the expression of two exemplary endogenous proliferation genes, MKI67 and TOP2A.

FIG. 4A depicts a clustered dotplot for scRNA sequencing of beta islet cell differentiation. FIG. 4B depicts a clustered dotplot for scRNA sequencing of cardiomyocyte differentiation.

FIG. 5A depicts a homology-directed repair (HDR) donor design for insertion of a nucleic acid encoding an agent into an endogenous proliferation gene locus. The agent can be, for example, a selection agent. FIG. 5B depicts an example of HDR introduction of iCaspase 9, cytosine deaminase, or a promotor-less GFP reporter into the 3′ UTR of the endogenous AURKB locus.

FIG. 6A depicts a graph of the knock-in (KI) efficiency of inducible Caspase 9-GFP (iC9-GFP) into candidate endogenous proliferation gene loci. KI efficiency was assessed by percentage of GFP-positive cells. KI efficiency of iC9-CD47 was assessed by percentage of CD47-positive cells. The lower dashed, horizontal line represents 2% of cells were GFP-positive or CD47-positive. The upper dashed, horizontal line represents 5% of cells were GFP-positive or CD47-positive. FIG. 6B depicts a graph of the clonal copy number by ddPCR. Plasmid backbone genomic copy number is plotted on the y-axis. bGH (polyA signal) genomic copy number is plotted on the x-axis. Integration of a nucleic acid encoding an agent was assessed by the bGH genomic copy number. Clones expressing monoallelic or biallelic integration (1 or 2 bGH genomic copy number) and without plasmid backbone (0 plasmid backbone genomic copy number) were selected as the “correct clones”. Clones with a plasmid backbone genomic copy number greater than 0 were excluded (labeled “excluded clones”).

FIG. 7 depicts the percentage of positivity for pluripotency markers. Each clone, which had iC9-GFP knocked into the specific locus, is marked as “mono” for monoallelic or “bi” for biallelic. Each set of bars from left to right represents the percentage of Oct4-positive, Sox2-positive, Nanog-positive, and Oct4/Sox2/Nanog-positive cells. The parental iPSC line (dko D11) and AAVS1 line (iC9-GFP knocked into the AAVS1 safe harbor site in iPSCs) are controls. dko D11 is a pluripotent cell line-derived clone with a double knock-out for genes B2M and CIITA.

FIG. 8A depicts the gene expression level of the specified, edited endogenous proliferation locus (locus with KI), compared to the gene's expression level in AAVS1-edited clones. The dashed, horizontal line represents the expression level of the specific endogenous proliferation gene in AAVS-1 edited clones. The expression level of all endogenous proliferation genes in AAVS1-edited clones is set to 1. FIG. 8B depicts the growth rate over 24 hours for the specified, edited endogenous proliferation locus (locus with KI). The parental iPSC line (dko D11) is the control. dko D11 is a pluripotent cell line-derived clone with a double knock-out for genes B2M and CIITA. Each edited locus is marked as “mono” for monoallelic and “bi” for biallelic.

FIGS. 9A-9B depicts AP20187 (also named “BB”) dosing kill curves of edited iPSCs. Twenty-four hours after the formation of embryoid bodies (EB), the EB were plated in DMEM, which was day 0 of the spontaneous differentiation timeline. Edited iPSCs at various points of an 18-day spontaneous differentiation timeline (undifferentiated iPSC and differentiated iPSCs at day 5, day 12, and day 18) were subjected to increasing concentrations of AP20187. Cell titer viability was determined 24 hours after dosing. FIG. 9A shows AP20187 dosing kill curves for iPSCs with iC9 KI at the AURKB locus (iC9_AURKB). FIG. 9B shows AP20187 dosing kill curves for iPSCs with iC9 KI at the CDK1 locus (iC9_CDK1). FIG. 9C shows AP20187 dosing kill curves for iPSCs with iC9 KI at the AAVS1 locus (iC9_AAVS1). iC9 AAVS1 is the control.

FIG. 10 depicts quantification of western blotting results for iC9 expression in the iC9_AURKB, iC9_CDK1, and dko D11 cell lines at the undifferentiated iPSC stage and day 14 (D14) of differentiation. The parental iPSC line (dko D11) is the control. dko D11 is a pluripotent cell line-derived clone with a double knock-out for genes B2M and CIITA.

FIGS. 11A-11M depict AP20187 (also named “BB dimerizer”) dosing kill curves of edited iPSCs. Twenty-four hours after the formation of embryoid bodies (EB), the EB were plated in DMEM, which was day 0 of the spontaneous differentiation timeline. Edited iPSCs at various points of an 18-day spontaneous differentiation timeline (undifferentiated iPSC and differentiated iPSCs at day 5, day 12, and day 18) were subjected to increasing concentrations of AP20187. Cell titer viability was determined 24 hours after dosing. FIG. 11A shows AP20187 dosing kill curves for iPSC clones with iC9-GFP KI at the CENPF locus. FIG. 11B shows AP20187 dosing kill curves for iPSC clones with iC9-GFP KI at the TOP2A locus. FIG. 11C shows AP20187 dosing kill curves for iPSC clones with iC9-GFP KI at the SMC4 locus. FIG. 11D shows AP20187 dosing kill curves for iPSC clones with iC9-GFP KI at the AURKB locus. FIG. 11E shows AP20187 dosing kill curves for iPSC clones with iC9-GFP KI at the CDK1 locus. FIG. 11F shows AP20187 dosing kill curves for iPSC clones with iC9-GFP KI at the BIRC5 locus. FIG. 11G shows AP20187 dosing kill curves for iPSC clones with iC9-GFP KI at the TPX2 locus. FIG. 11H shows AP20187 dosing kill curves for iPSC clones with iC9-GFP KI at the MKI67 locus. FIG. 11I shows AP20187 dosing kill curves for iPSC clones with iC9-GFP KI at the CDC20 locus. FIG. 11J shows AP20187 dosing kill curves for iPSC clones with iC9-GFP KI at the PTTG1 locus. FIG. 11K shows AP20187 dosing kill curves for iPSC clones with iC9-GFP KI at the KIF11 locus. FIG. 11L shows AP20187 dosing kill curves for iPSC clones with iC9-GFP KI at the CENPK locus. FIG. 11M shows AP20187 dosing kill curves for iPSC clones with iC9-GFP KI at the TYMS locus. For the graphs with a horizontal line at about 100% cell viability with circles to denote the data points, the horizontal line represents the parental iPSC line (dko D11) control. dko D11 is a pluripotent cell line-derived clone with a double knock-out for genes B2M and CIITA.

FIG. 12 is a plot of the IC₅₀ for a specific proliferation gene KI clone against the iC9 expression as measured by mean fluorescence intensity (MFI) of GFP. The dotted horizontal line represents incomplete killing. The dotted vertical line represents the minimum threshold for iC9-GFP expression. The 2 in parenthesis denotes biallelic. All other loci are monoallelic.

FIG. 13 is a bar graph of the growth rate over 24 hours of specific iC9 KI clones on day 0, day 5, and day 18 of differentiation. For each day, the bars from left to right are as follows: dko D11 (parental iPSC control), AVSS1 (monoallelic), AAVS1 (biallelic), CENPF (biallelic), TOP2A (monoallelic), TOP2A (biallelic), SMC4 (monoallelic), AURKB (monoallelic), AURKB (biallelic), CDK1 (monoallelic), BIRC5 (biallelic), TPX2 (monoallelic), MKI67 (biallelic), CDC20 (monoallelic), PTTG1 (monoallelic), KIF11 (monoallelic), and TYMS (monoallelic). dko D11 is a pluripotent cell line-derived clone with a double knock-out for genes B2M and CIITA.

FIGS. 14A-14O depict AP20187 (also named “BB dimerizer”) dosing kill curves of edited iPSCs with iC9 inserted at proliferation loci. Twenty-four hours after the formation of embryoid bodies (EB), the EB were plated in DMEM, which was day 0 of the spontaneous differentiation timeline. Edited iPSCs at various points (undifferentiated iPSC, differentiated iPSCs at day 5, and differentiated iPSCs at day 18) of an 18-day spontaneous differentiation timeline were subjected to increasing concentrations of AP20187. Cell titer viability was determined 24 hours after dosing. FIG. 14A shows AP20187 dosing kill curves for the parental iPSC control (dko), AAVS1 monoallelic KI clone A (control), and AAVS1 biallelic KI clone B (control). dko D11 is a pluripotent cell line-derived clone with a double knock-out for genes B2M and CIITA. FIG. 14B shows AP20187 dosing kill curves for CDK1 monoallelic KI clones A and B. FIG. 14C shows AP20187 dosing kill curves for CENPF biallelic KI clones A and B. FIG. 14D shows AP20187 dosing kill curves for TOP2A monoallelic KI clones A and B. FIG. 14E shows AP20187 dosing kill curves for TOP2A biallelic KI clones C and D. FIG. 14F shows AP20187 dosing kill curves for SMC4 monoallelic KI clones A and B. FIG. 14G shows AP20187 dosing kill curves for AURKB monoallelic KI clones A and B. FIG. 14H shows AP20187 dosing kill curves for AURKB biallelic KI clones C and D. FIG. 14I shows AP20187 dosing kill curves for BIRC5 biallelic KI clone A. FIG. 14J shows AP20187 dosing kill curves for TPX2 monoallelic KI clones A and B. FIG. 14K shows AP20187 dosing kill curves for MKI67 biallelic KI clones A and B. FIG. 14L shows AP20187 dosing kill curves for CDC20 monoallelic KI clones A and B. FIG. 14M shows AP20187 dosing kill curves for PTTG1 monoallelic KI clones A and B. FIG. 14N shows AP20187 dosing kill curves for KIF11 monoallelic KI clone A. FIG. 14O shows AP20187 dosing kill curves for TYMS monoallelic KI clone A.

FIGS. 15A and 15B is a graph plotting the IC₅₀ for the kill curves (bars) against the percentage of resistant cells on day 18 (triangles) of the specified proliferation gene locus. The vertical dashed line in FIG. 15B delineates gene loci that were further investigated (left of vertical dashed line) and gene loci that did not fit the criteria (right of the vertical dashed line). The 2 in parenthesis denotes biallelic. All other loci are monoallelic.

FIGS. 16A-16C depict 5-flurocytosine (5-FC) dosing kill curves of edited iPSCs and pancreatic progenitors. Cytosine deaminase (CDA) was knocked into the AAVS1, AURKB, or CDK1 loci in iPSCs that were differentiated into pancreatic progenitors. Undifferentiated iPSCs and day 12 differentiated pancreatic progenitors (“D12”) were subjected to increasing concentrations of 5-FC. Cell titers were assessed 72 hours after dosing. FIG. 16A shows 5-FC dosing kill curves for CDA_AURKB iPSCs and CDA_AURKB pancreatic progenitors. FIG. 16B shows 5-FC dosing kill curves for CDA_CDK1 iPSCs and CDA_CDK1 pancreatic progenitors. FIG. 16C shows 5-FC dosing kill curves for the following controls: dko parental iPSC line (a pluripotent cell line-derived clone with a double knock-out for genes B2M and CIITA), dko pancreatic progenitors (day 12 pancreatic progenitors differentiated from the dko parental iPSC line), CDA_AAVS1 iPSCs (CDA knocked into the AAVS1 safe harbor site in iPSCs), and CDA_AAVS1 pancreatic progenitors (CDA knocked into the AAVS1 safe harbor site in pancreatic progenitors).

FIGS. 17A-17D depict flow cytometry histograms of Ki67-positive cells in pancreatic progenitors 72 hours after dosing with or without 100 μM 5-FC. CDA was knocked into the AAVS1 (CDA_AAVS1), AURKB (CDA_AURKB), or CDK1 (CDA_CDK1) loci in pancreatic progenitors, and pancreatic progenitors at day 12 of beta islet differentiation were used. FIG. 17A shows a comparison of Ki67-positive cell count in untreated and treated CDA_AURKB pancreatic progenitor cultures. FIG. 17B shows a comparison of Ki67-positive cell count in untreated and treated CDA_CDK1 pancreatic progenitor cultures. FIG. 17C shows a comparison of Ki67-positive cell count in untreated and treated CDA_AAVS1 pancreatic progenitor cultures (control). FIG. 17D shows a comparison of Ki67-positive cell count in untreated and treated dko pancreatic progenitor cultures (control).

FIGS. 18A-18C depict AP20187 (also named “BB dimerizer”) dosing kill curves of edited iPSCs and pancreatic progenitors. iCaspase 9 was knocked into the AAVS1, AURKB, or CDK1 loci in iPSCs that were differentiated into pancreatic progenitors, and undifferentiated iPSCs and day 12 differentiated pancreatic progenitors (“D12”) were subjected to increasing concentrations of AP20187. Cell titers were assessed 48 hours after dosing. FIG. 18A shows AP20187 dosing kill curves for iC9_AURKB iPSCs and iC9_AURKB pancreatic progenitors. FIG. 18B shows AP20187 dosing kill curves for iC9_CDK1 iPSCs and iC9_CDK1 pancreatic progenitors. FIG. 18C shows AP20187 dosing kill curves for the following controls: dko parental iPSC line (dko D11 is a pluripotent cell line-derived clone with a double knock-out for genes B2M and CIITA), dko pancreatic progenitors (double knock-out for genes B2M and CIITA in pancreatic progenitors), iC9_AAVS1 iPSCs (iC9 knocked into the AAVS1 safe harbor site in iPSCs), and iC9_AAVS1 pancreatic progenitors (iC9 knocked into the AAVS1 safe harbor site in pancreatic progenitors).

FIG. 19 depicts a clustered dotplot for scRNA sequencing of beta islet cell differentiation. Top ductal cell markers are identified.

FIG. 20 depicts a homology-directed repair (HDR) donor design for insertion of a nucleic acid encoding an agent into an off-target cell marker gene locus. The agent can be, for example, a selection agent.

FIG. 21 depicts a homology-directed repair design for insertion of a nucleic acid encoding an agent into an endogenous proliferation gene or off-target cell marker gene locus. The agent can be, for example, a detection agent.

### DETAILED DESCRIPTION OF THE INVENTION

The present disclosure relates to modified cells comprising a nucleic acid encoding an agent inserted at an endogenous proliferation gene or off-target cell marker gene, as well as compositions, methods, uses, and kits related thereto.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The term “locus” refers to a fixed position on a chromosome where a particular gene or genetic marker is located. Reference to a “target locus” refers to a particular locus of a desired gene in which it is desired to target a genetic modification, such as a gene edit or integration of an exogenous polynucleotide.

As used herein, the term “and/or” includes any and all combinations of one or more of the associated listed items.

The term “expression” with reference to a gene or “gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or can be a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation. Hence, reference to expression or gene expression includes protein (or polypeptide) expression or expression of a transcribable product of a gene such as mRNA. The protein expression may include intracellular expression or surface expression of a protein. Typically, expression of a gene product, such as mRNA or protein, is at a level that is detectable in the cell.

As used herein, the term “exogenous” with reference to a polypeptide or a polynucleotide is intended to mean that the referenced molecule is introduced into the cell of interest. The exogenous molecule, such as exogenous polynucleotide, exogenous sequence, or exogenous transgene, can be introduced, for example, by introduction of an exogenous encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell. In some cases, an “exogenous” molecule is a molecule, construct, factor and the like that is not normally present in a cell but can be introduced into a cell by one or more genetic, biochemical, or other methods.

The term “endogenous” refers to a referenced molecule, such as a polynucleotide (e.g., gene), or polypeptide, that is present in a native or unmodified cell. For instance, the term when used in reference to expression of an endogenous gene refers to expression of a gene encoded by an endogenous nucleic acid contained within the cell and not exogenously introduced. A “gene,” includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. The sequence of a gene is typically present at a fixed chromosomal position or locus on a chromosome in the cell.

The term “operably linked” refers to two or more genetic elements, such as a polynucleotide coding sequence and a promoter, placed in relative positions that permit the proper biological functioning of the elements, such as the promoter directing transcription of the coding sequence.

The term “wild-type” or “WT” as used herein, refers to an entity having a structure and/or activity as found in nature in a “normal” (as contrasted with mutant, diseased, altered, engineered, etc.) state or context. A wild-type amino acid sequence is an amino acid sequence that is found in nature, including allelic variations. A wild-type protein or polypeptide has an amino acid sequence that has not been intentionally modified.

As used herein, the term “modification” with reference to a cell refers to any change or alteration of a nucleic acid in the genome of a cell, which may impact gene expression in the cell. For example, a modification includes a genetic modification that results in alterations, additions, and/or deletion of genes or portions of genes or other nucleic acid sequences. A modified cell, such as a genetically modified cell, can also refer to a cell with an added, deleted and/or altered gene or portion of a gene. In some embodiments, the modification is a genetic modification that directly changes the gene or regulatory elements thereof encoding a protein product in a cell, such as by gene editing, mutagenesis or by genetic engineering of an exogenous polynucleotide or transgene. Genetic modifications include, for example, both transient knock-in or knock-down mechanisms, and mechanisms that result in permanent knock-in, knock-down, or knock-out of target genes or portions of genes or nucleic acid sequences. Genetic modifications include, for example, both transient knock-in and mechanisms that result in permanent knock-in of nucleic acids sequences. Genetic modifications also include, for example, reduced or increased transcription, reduced or increased mRNA stability, reduced or increased translation, and reduced or increased protein stability.

As used herein, the terms “effective amount” and “pharmaceutically effective amount” refer to a nontoxic but sufficient amount of an agent or drug to provide the desired biological result. That result can be reduction and/or alleviation of the signs, symptoms, or causes of a disease or disorder, imaging or monitoring of an in vitro or in vivo system (including a living organism), or any other desired alteration of a biological system. An appropriate effective amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one particle with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

A “disease” or “disorder” as used herein refers to a condition where treatment is needed and/or desired.

As used herein, the terms “treat,” “treating,” or “treatment” refer to ameliorating a disease or disorder, e.g., slowing or arresting or reducing the development of the disease or disorder or reducing at least one of the clinical symptoms thereof. For purposes of this disclosure, ameliorating a disease or disorder can include obtaining a beneficial or desired clinical result that includes, but is not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

The terms “individual” and “subject” are used interchangeably herein to refer to an animal, for example a mammal. The term patient includes human and veterinary subjects. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. The subject can be male or female and can be any suitable age, including infant, juvenile, adolescent, adult, and geriatric subjects. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder. In particular embodiments, the subject is a human, such as a human patient.

The term “donor subject” or “donor individual” refers to an animal, for example, a human from whom cells can be obtained. The “non-human animals” and “non-human mammals” as used interchangeably herein, includes mammals such as rats, mice, rabbits, sheep, cats, dogs, cows, pigs, and non-human primates. The term “donor subject” also encompasses any vertebrate including but not limited to mammals, reptiles, amphibians and fish. However, advantageously, the donor subject is a mammal such as a human, or other mammals such as a domesticated mammal, e.g., dog, cat, horse, and the like, or production mammal, e.g., cow, sheep, pig, and the like. A “donor subject” can also refer to more than one donor, for example one or more humans or non-human animals or non-human mammals.

The term “recipient”, “recipient patient”, or “recipient individual” refers to an animal, for example, a human to whom treatment, including prophylactic treatment, with the cells as described herein, is provided. For treatment of those infections, conditions, or disease states, which are specific for a specific animal such as a human patient, the term patient refers to that specific animal. The term “recipient patient” also encompasses any vertebrate including but not limited to mammals, reptiles, amphibians, and fish. However, advantageously, the recipient patient is a mammal such as a human, or other mammals such as a domesticated mammal, e.g., dog, cat, horse, and the like, or production mammal, e.g., cow, sheep, pig, and the like.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

As used herein, “percent (%) nucleic acid sequence identity” and “homology” with respect to a nucleic acid, nucleotide, DNA, or RNA sequence are defined as the percentage of nucleotides in a candidate sequence that are identical with the nucleotides in the specific nucleic acid, DNA, or RNA sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “comparable”, as used herein, refers to two or more agents, entities, situations, sets of conditions, etc. that may not be identical to one another but that are sufficiently similar to permit comparison there between so that conclusions may reasonably be drawn based on differences or similarities observed. Persons of ordinary skill in the art will understand, in context, what degree of identity is required in any given circumstance for two or more such agents, entities, situations, sets of conditions, etc. to be considered comparable. A control cell, for example, can be a comparable cell (e.g., same cell type) that does not comprise the relative modifications.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “antibody” is used to denote, in addition to natural antibodies, genetically engineered or otherwise modified forms of immunoglobulins or portions thereof, including chimeric antibodies, human antibodies, humanized antibodies, or synthetic antibodies. The antibodies may be monoclonal or polyclonal antibodies. In those embodiments wherein an antibody is an immunogenically active portion of an immunoglobulin molecule, the antibody may include, but is not limited to, a single chain variable fragment antibody (scFv), disulfide linked Fv, single domain antibody (sdAb), VHH antibody, antigen-binding fragment (Fab), Fab′, F(ab′)2 fragment, or diabody. An scFv antibody is derived from an antibody by linking the variable regions of the heavy (VH) and light (VL) chains of the immunoglobulin with a short linker peptide. Similarly, a disulfide linked Fv antibody can be generated by linking the VH and VL using an interdomain disulfide bond. On the other hand, sdAbs consist of only the variable region from either the heavy or light chain and usually are the smallest antigen-binding fragments of antibodies. A VHH antibody is the antigen binding fragment of heavy chain only. A diabody is a dimer of scFv fragment that consists of the VH and VL regions noncovalent connected by a small peptide linker or covalently linked to each other. The antibodies disclosed herein, including those that comprise an immunogenically active portion of an immunoglobulin molecule, retain the ability to bind a specific antigen.

The term “antigen”, as used herein, refers to a molecule capable of provoking an immune response. Antigens include but are not limited to cells, cell extracts, proteins, polypeptides, peptides, polysaccharides, polysaccharide conjugates, peptide and non-peptide mimics of polysaccharides and other molecules, small molecules, lipids, glycolipids, carbohydrates, viruses and viral extracts and multicellular organisms such as parasites and allergens. The term antigen broadly includes any type of molecule which is recognized by a host immune system as being foreign.

A “binding domain,” also referred to as a “binding region,” refers to an antibody or portion thereof that possesses the ability to specifically and non-covalently associate, unite, or combine with a target. A binding domain includes any naturally occurring, synthetic, semi-synthetic, or recombinantly produced binding partner for a biological molecule, a molecular complex, or other target of interest. Exemplary binding domains include receptor ectodomains, ligands, scFvs, disulfide linked Fvs, sdAbs, VHH antibodies, Fab fragments, Fab′ fragments, F(ab′)2 fragments, diabodies, or other synthetic polypeptides selected for their specific ability to bind to a biological molecule, a molecular complex, or other target of interest.

“Immune signaling molecule” as used herein refers to, in some cases, a molecule, protein, peptide and the like that activates immune signaling pathways.

As used herein, “fusogen” refers to an agent or molecule that creates an interaction between two membranes, including membrane enclosed lumens. In embodiments, the fusogen facilitates fusion of the membranes. In other embodiments, the fusogen creates a connection, e.g., a pore, between two membranes or lumens (e.g., a lumen of a retroviral particle and a cytoplasm of a target cell). In some embodiments, the fusogen comprises a complex of two or more proteins, e.g., wherein neither protein has fusogenic activity alone. In some embodiments, the fusogen comprises a targeting domain.

The term “tolerogenic factor” as used herein include immunosuppressive factors or immune-regulatory factors that modulate or affect the ability of a cell to be recognized by the immune system of a host or recipient subject upon administration, transplantation, or engraftment. Typically, a tolerogenic factor is a factor that induces immunological tolerance to a modified primary cell or stem cell (or cell derived from a stem cell) so that the modified primary cell or stem cell (or cell derived from a stem cell) is not targeted, such as rejected, by the host immune system of a recipient. Hence, a tolerogenic factor may be a hypoimmunity factor. Examples of tolerogenic factors include immune cell inhibitory receptors (e.g., CD47), proteins that engage immune cell inhibitory receptors, checkpoint inhibitors and other molecules that reduce innate or adaptive immune recognition.

The term “hypoimmunogenic” refers to a cell that is less prone to immune rejection by a subject to which such cells are transplanted. For example, relative to a comparable cell that does not contain certain modifications (e.g., a wild-type cell or a cell without one or more modifications intended to address immunogenicity (e.g., a modification to an HLA-I, HLA-II and/or tolerogenic factor gene)), such a hypoimmunogenic cell may be about 2.5%, 5%, 10%, 20%, 30%, 40%, 50%, 60%, 70%, 80%, 90%, 95%, 97.5%, 99% or more less prone to immune rejection by a subject into which such cells are transplanted. Typically, the hypoimmunogenic cells are allogenic to the subject and a hypoimmunogenic cell evades immune rejection in an MHC-mismatched allogeneic recipient. In some embodiments, a hypoimmunogenic cell is protected from T cell-mediated adaptive immune rejection and/or innate immune cell rejection.

As used herein, “hypoimmunogenicity” of a cell can be determined by evaluating the immunogenicity of the cell such as the cell's ability to elicit adaptive and innate immune responses.

The term “kill switch” refers to a system for expressing a protein linked to a gene of interest that, when the gene of interest is expressed or upregulated and when an inducer is provided, the cell expressing the gene of interest will be eliminated. An activated kill switch leads to clearance or death of the cell, e.g., through apoptosis. A kill switch can be designed to be or include an exogenous molecule administered to prevent or mitigate an adverse clinical event. A kill switch may include a protein or molecule that allows for the control of cellular activity in response to an adverse event. A kill switch may be used to eliminate cells that overproliferate or cells that are of an unwanted cell type. A kill switch can be expressed in an inactive state and is fatal to a cell expressing the kill switch upon activation of the switch by a selective, externally provided inducer. In some embodiments, the selection agent is a kill switch. In some embodiments, the kill switch gene is cis-acting in relation to the gene of interest in a construct. Activation of the kill switch causes the cell to kill solely itself or itself and neighboring cells through apoptosis or necrosis.

The term “cell” includes the subject cell and its progeny.

It will be understood by one of ordinary skill in the art that uracil and thymine can both be represented by ‘t’, instead of ‘u’ for uracil and ‘t’ for thymine; in the context of a ribonucleic acid, it will be understood that ‘t’ is used to represent uracil unless otherwise indicated.

It is understood that embodiments of the invention described herein include “consisting” and/or “consisting essentially of” embodiments.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods. Reference to “about” a value or parameter herein includes (and describes) variations that are directed to that value or parameter per se. For example, description referring to “about X” includes description of “X”.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For instance, where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure. In some embodiments, two opposing and open-ended ranges are provided for a feature, and in such description, it is envisioned that combinations of those two ranges are provided herein. For example, in some embodiments, it is described that a feature is greater than about 10 units, and it is described (such as in another sentence) that the feature is less than about 20 units, and thus, the range of about 10 units to about 20 units is described herein.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

Before the technology is further described, it is to be understood that this technology is not limited to particular embodiments described, as such may, of course, vary. It is also to be understood that the terminology used herein is for the purpose of describing particular embodiments only, and is not intended to be limiting, since the scope of the present disclosure will be limited only by the appended claims. It should also be understood that the headers used herein are not limiting and are merely intended to orient the reader, but the subject matter generally applies to the technology disclosed herein.

### II. Cells

Certain aspects of the present disclosure relate to modified cells comprising a nucleic acid encoding a selection agent or a detection agent integrated at a gene of interest locus and operably linked to the promoter of the gene of interest, wherein expression of the nucleic acid encoding the selection agent or the detection agent is regulated by the promoter of the gene of interest and/or regulatory elements of the gene of interest. In some aspects, when the promoter of the gene of interest is “turned on” the selection or detection agent is expressed allowing selection or detection of certain cell types.

### A. Endogenous Proliferation Genes

When normal tissue and tumor samples are compared by microarray or sequencing analysis, the biggest differences most often occur in the expression levels of genes that control cell proliferation. Cell proliferation is an increase in cell number due to cell division. Although cell proliferation is necessary for normal tissue development and maintenance over a lifespan and is a tightly regulated process, with many different proteins controlling cell cycle checkpoints, genetic mutations found in cancer cells can cause uncontrolled cellular proliferation. Accordingly, various endogenous proliferation genes are upregulated in cancer cells. Proliferation genes can be screened using next-generation sequencing methods known in the art, and are described in, e.g., Metzker, M. (2010) Nature Biotechnology Reviews 11:31-46, which is incorporated herein by reference. In some instances, the sequencing may comprise, for example, bulk RNA-sequencing or single-cell RNA sequencing. Endogenous proliferation genes may be identified by sequencing iPSC cells versus differentiated cells, identifying a subset of genes with largest fold change between datasets, and cross-referencing with open databases of essential cancer genes to identify gene candidates that are essential to proliferation but not expressed in terminally differentiated cell types of interest. The genes that are strong markers of proliferation (overexpressed in iPSC cells) but are not essential are identified as endogenous proliferation genes.

In some embodiments, there is provided a modified cell comprising a nucleic acid encoding a selection agent or a detection agent integrated at an endogenous proliferation gene locus and operably linked to the promoter of the endogenous proliferation gene, wherein expression of the nucleic acid encoding the selection agent or the detection agent is regulated by the promoter of the endogenous proliferation gene and/or regulatory elements of the endogenous proliferation gene. In some embodiments, there is provided a modified cell comprising a nucleic acid encoding a selection agent and a detection agent integrated at an endogenous proliferation gene locus and operably linked to the promoter of the endogenous proliferation gene, wherein expression of the nucleic acid encoding the selection agent and the detection agent is regulated by the promoter of the endogenous proliferation gene and/or regulatory elements of the endogenous proliferation gene. In some embodiments, there is provided a modified cell comprising a nucleic acid encoding a first detection agent and a second detection agent, wherein the nucleic acid is integrated at an endogenous proliferation gene locus and operably linked to the promoter of the endogenous proliferation gene, and wherein expression of the nucleic acid encoding the first detection agent and the second detection agent is regulated by the promoter of the endogenous proliferation gene and/or regulatory elements of the endogenous proliferation gene. In some embodiments, there is provided a modified cell comprising a nucleic acid encoding a first selection agent and a second selection agent, wherein the nucleic acid is integrated at an endogenous proliferation gene locus and operably linked to the promoter of the endogenous proliferation gene, and wherein expression of the nucleic acid encoding the first selection agent and the second selection agent is regulated by the promoter of the endogenous proliferation gene and/or regulatory elements of the endogenous proliferation gene. In some embodiments, the first selection agent and the second selection agent are operably linked. In some embodiments, the first selection agent and the second selection agent are operably linked by an internal ribosome entry site. In some embodiments, the first selection agent and the second selection agent are operably linked by a self-cleaving peptide. In some embodiments, there is provided a modified cell comprising a first nucleic acid encoding a selection agent and a second nucleic acid encoding a detection agent, wherein the first nucleic acid is integrated at a first endogenous proliferation gene locus and operably linked to the promoter of the first endogenous proliferation gene, wherein expression of the first nucleic acid encoding the selection agent is regulated by the promoter of the first endogenous proliferation gene and/or regulatory elements of the first endogenous proliferation gene, wherein the second nucleic acid is integrated at a second endogenous proliferation gene locus and operably linked to the promoter of the second endogenous proliferation gene, and wherein expression of the second nucleic acid encoding the detection agent is regulated by the promoter of the second endogenous proliferation gene and/or regulatory elements of the endogenous proliferation gene. In some embodiments, there is provided a modified cell comprising a first nucleic acid encoding a first selection agent and a second nucleic acid encoding a second selection agent, wherein the first nucleic acid is integrated at a first endogenous proliferation gene locus and operably linked to the promoter of the first endogenous proliferation gene, wherein expression of the first nucleic acid encoding the first selection agent is regulated by the promoter of the first endogenous proliferation gene and/or regulatory elements of the first endogenous proliferation gene, wherein the second nucleic acid is integrated at a second endogenous proliferation gene locus and operably linked to the promoter of the second endogenous proliferation gene, and wherein expression of the second nucleic acid encoding the second selection agent is regulated by the promoter of the second endogenous proliferation gene and/or regulatory elements of the endogenous proliferation gene. In some embodiments, there is provided a modified cell comprising a first nucleic acid encoding a first detection agent and a second nucleic acid encoding a second detection agent, wherein the first nucleic acid is integrated at a first endogenous proliferation gene locus and operably linked to the promoter of the first endogenous proliferation gene, wherein expression of the first nucleic acid encoding the first detection agent is regulated by the promoter of the first endogenous proliferation gene and/or regulatory elements of the first endogenous proliferation gene, wherein the second nucleic acid is integrated at a second endogenous proliferation gene locus and operably linked to the promoter of the endogenous proliferation gene, and wherein expression of the second nucleic acid encoding the second detection agent is regulated by the promoter of the second endogenous proliferation gene and/or regulatory elements of the second endogenous proliferation gene. In some embodiments, the first endogenous proliferation gene and/or the second endogenous proliferation gene are expressed in a cell that is proliferating. In some embodiments, the first endogenous proliferation gene and/or the second endogenous proliferation gene are expressed in a cell that is proliferating. In some embodiments, the first endogenous proliferation gene and/or the second endogenous proliferation gene are expressed in a cell that is included in a population of therapeutic cells. In some embodiments, the first endogenous proliferation gene and/or the second endogenous proliferation gene are expressed in a cell that is included in a population of therapeutic cells but is not a therapeutic cell. In some embodiments, the cell is a pluripotent cell, a progenitor cell, or a differentiated cell that is proliferating. In some embodiments, the first endogenous proliferation gene and the second endogenous proliferation gene are the same, and the first and second nucleic acids are integrated at different alleles. In some embodiments, the first endogenous proliferation gene and the second endogenous proliferation gene are different. In some embodiments, the endogenous proliferation gene is expressed at a higher level from day 0 to about day 5 of differentiation, compared to expression of the endogenous proliferation gene at day 18 of differentiation, or wherein the endogenous proliferation gene is highly expressed from day 1 to about day 5 of differentiation. In some embodiments, the nucleic acid encoding the selection agent or the detection agent is integrated in an exon or an intron of the endogenous proliferation gene. In some embodiments, the nucleic acid encoding the selection agent or the detection agent is integrated in a 5′ UTR or a 3′ UTR of the endogenous proliferation gene. In some embodiments, following integration, the endogenous proliferation gene locus comprises i) nucleic acid encoding the endogenous proliferation gene, ii) an internal ribosomal entry site or a self-cleaving RNA site, and iii) the nucleic acid encoding the selection agent or a detection agent. Examples of endogenous proliferation genes include, but are not limited to, AURKB, CDK1, CDC20, RRM2, BIRC5, TOP2A, PTTG1, CCNB1, TPX2, KIF11, SPC25, CENPK, SMC4, TYMS, H2AZ1, TMSB15A, CENPF, and MKI67.

In some embodiments, the endogenous proliferation gene is AURKB. In some embodiments, the nucleic acid encoding a selection agent or a detection agent is integrated at an exon of AURKB, such as any one of exons 1, 2, 3, 4, 5, 6, 7, 8, or 9. In some embodiments, the nucleic acid encoding a selection agent or a detection agent is integrated at an intron of AURKB, such as any one of introns 1, 2, 3, 4, 5, 6, 7, or 8. In some embodiments, the nucleic acid encoding the selection agent or the detection agent is integrated in the 5′ UTR of AURKB. In some embodiments, the nucleic acid encoding the selection agent or the detection agent is integrated in the 3′ UTR of AURKB.

In some embodiments, the endogenous proliferation gene is CDK1. In some embodiments, the nucleic acid encoding a selection agent or a detection agent is integrated at an exon of CDK1, such as any one of exons 1, 2, 3, 4, 5, 6, 7, or 8. In some embodiments, the nucleic acid encoding a selection agent or a detection agent is integrated at an intron of CDK1, such as any one of introns 1, 2, 3, 4, 5, 6, or 7. In some embodiments, the nucleic acid encoding the selection agent or the detection agent is integrated in the 5′ UTR of CDK1. In some embodiments, the nucleic acid encoding the selection agent or the detection agent is integrated in the 3′ UTR of CDK1.

In some embodiments, the endogenous proliferation gene is CDC20. In some embodiments, the nucleic acid encoding a selection agent or a detection agent is integrated at an exon of CDC20, such as any one of exons 1, 2, 3, 4, 5, 6, 7, 8, 9, 10, or 11. In some embodiments, the nucleic acid encoding a selection agent or a detection agent is integrated at an intron of CDC20, such as any one of introns 1, 2, 3, 4, 5, 6, 7, 8, 9, or 10. In some embodiments, the nucleic acid encoding the selection agent or the detection agent is integrated in the 5′ UTR of CDC20. In some embodiments, the nucleic acid encoding the selection agent or the detection agent is integrated in the 3′ UTR of CDC20.

In some embodiments, the endogenous proliferation gene is RRM2. In some embodiments, the nucleic acid encoding a selection agent or a detection agent is integrated at an exon of RRM2, such as any one of exons 1, 2, 3, 4, 5, 6, 7, 8, 9, or 10. In some embodiments, the nucleic acid encoding a selection agent or a detection agent is integrated at an intron of RRM2, such as any one of introns 1, 2, 3, 4, 5, 6, 7, 8, or 9. In some embodiments, the nucleic acid encoding the selection agent or the detection agent is integrated in the 5′ UTR of RRM2. In some embodiments, the nucleic acid encoding the selection agent or the detection agent is integrated in the 3′ UTR of RRM2.

In some embodiments, the endogenous proliferation gene is BIRC5. In some embodiments, the nucleic acid encoding a selection agent or a detection agent is integrated at an exon of BIRC5, such as any one of exons 1, 2, 3, or 4. In some embodiments, the nucleic acid encoding a selection agent or a detection agent is integrated at an intron of BIRC5, such as any one of introns 1, 2, or 3. In some embodiments, the nucleic acid encoding the selection agent or the detection agent is integrated in the 5′ UTR of BIRC5. In some embodiments, the nucleic acid encoding the selection agent or the detection agent is integrated in the 3′ UTR of BIRC5.

In some embodiments, the endogenous proliferation gene is TOP2A. In some embodiments, the nucleic acid encoding a selection agent or a detection agent is integrated at an exon of TOP2A, such as any one of exons 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35. In some embodiments, the nucleic acid encoding a selection agent or a detection agent is integrated at an intron of TOP2A, such as any one of introns 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34. In some embodiments, the nucleic acid encoding the selection agent or the detection agent is integrated in the 5′ UTR of TOP2A. In some embodiments, the nucleic acid encoding the selection agent or the detection agent is integrated in the 3′ UTR of TOP2A.

In some embodiments, the endogenous proliferation gene is PTTG1. In some embodiments, the nucleic acid encoding a selection agent or a detection agent is integrated at an exon of PTTG1, such as any one of exons 1, 2, 3, 4, 5, or 6. In some embodiments, the nucleic acid encoding a selection agent or a detection agent is integrated at an intron of PTTG1, such as any one of introns 1, 2, 3, 4, or 5. In some embodiments, the nucleic acid encoding the selection agent or the detection agent is integrated in the 5′ UTR of PTTG1. In some embodiments, the nucleic acid encoding the selection agent or the detection agent is integrated in the 3′ UTR of PTTG1.

In some embodiments, the endogenous proliferation gene is CCNB1. In some embodiments, the nucleic acid encoding a selection agent or a detection agent is integrated at an exon of CCNB1, such as any one of exons 1, 2, 3, 4, 5, 6, 7, 8, or 9. In some embodiments, the nucleic acid encoding a selection agent or a detection agent is integrated at an intron of CCNB1, such as any one of introns 1, 2, 3, 4, 5, 6, 7, or 8. In some embodiments, the nucleic acid encoding the selection agent or the detection agent is integrated in the 5′ UTR of CCNB1. In some embodiments, the nucleic acid encoding the selection agent or the detection agent is integrated in the 3′ UTR of CCNB1.

In some embodiments, the endogenous proliferation gene is TPX2. In some embodiments, the nucleic acid encoding a selection agent or a detection agent is integrated at an exon of TPX2, such as any one of exons 1, 2, 3, 4, 5, 6, 7, 8, 9, 10, 11, 12, 13, 14, 15, 16, 17, or 18. In some embodiments, the nucleic acid encoding a selection agent or a detection agent is integrated at an intron of TPX2, such as any one of introns 1, 2, 3, 4, 5, 6, 7, 8, 9, 10, 11, 12, 13, 14, 15, 16, or 17. In some embodiments, the nucleic acid encoding the selection agent or the detection agent is integrated in the 5′ UTR of TPX2. In some embodiments, the nucleic acid encoding the selection agent or the detection agent is integrated in the 3′ UTR of TPX2.

In some embodiments, the endogenous proliferation gene is KIF11. In some embodiments, the nucleic acid encoding a selection agent or a detection agent is integrated at an exon of KIF11, such as any one of exons 1, 2, 3, 4, 5, 6, 7, 8, 9, 10, 11, 12, 13, 14, 15, 16, 17, 18, 19, 20, 21, or 22. In some embodiments, the nucleic acid encoding a selection agent or a detection agent is integrated at an intron of KIF11, such as any one of introns 1, 2, 3, 4, 5, 6, 7, 8, 9, 10, 11, 12, 13, 14, 15, 16, 17, 18, 19, 20, or 21. In some embodiments, the nucleic acid encoding the selection agent or the detection agent is integrated in the 5′ UTR of KIF11. In some embodiments, the nucleic acid encoding the selection agent or the detection agent is integrated in the 3′ UTR of KIF11.

In some embodiments, the endogenous proliferation gene is SPC25. In some embodiments, the nucleic acid encoding a selection agent or a detection agent is integrated at an exon of SPC25, such as any one of exons 1, 2, 3, 4, 5, 6, or 7. In some embodiments, the nucleic acid encoding a selection agent or a detection agent is integrated at an intron of SPC25, such as any one of introns 1, 2, 3, 4, 5, or 6. In some embodiments, the nucleic acid encoding the selection agent or the detection agent is integrated in the 5′ UTR of SPC25. In some embodiments, the nucleic acid encoding the selection agent or the detection agent is integrated in the 3′ UTR of SPC25.

In some embodiments, the endogenous proliferation gene is CENPK. In some embodiments, the nucleic acid encoding a selection agent or a detection agent is integrated at an exon of CENPK, such as any one of exons 1, 2, 3, 4, 5, 6, 7, 8, 9, 10, or 11. In some embodiments, the nucleic acid encoding a selection agent or a detection agent is integrated at an intron of CENPK, such as any one of introns 1, 2, 3, 4, 5, 6, 7, 8, 9, or 10. In some embodiments, the nucleic acid encoding the selection agent or the detection agent is integrated in the 5′ UTR of CENPK. In some embodiments, the nucleic acid encoding the selection agent or the detection agent is integrated in the 3′ UTR of CENPK.

In some embodiments, the endogenous proliferation gene is SMC4. In some embodiments, the nucleic acid encoding a selection agent or a detection agent is integrated at an exon of SMC4, such as any one of exons 1, 2, 3, 4, 5, 6, 7, 8, 9, 10, 11, 12, 13, 14, 15, 16, 17, 18, 19, 20, 21, 22, 23, or 24. In some embodiments, the nucleic acid encoding a selection agent or a detection agent is integrated at an intron of SMC4, such as any one of introns 1, 2, 3, 4, 5, 6, 7, 8, 9, 10, 11, 12, 13, 14, 15, 16, 17, 18, 19, 20, 21, 22, or 23. In some embodiments, the nucleic acid encoding the selection agent or the detection agent is integrated in the 5′ UTR of SMC4. In some embodiments, the nucleic acid encoding the selection agent or the detection agent is integrated in the 3′ UTR of SMC4.

In some embodiments, the endogenous proliferation gene is TYMS. In some embodiments, the nucleic acid encoding a selection agent or a detection agent is integrated at an exon of TYMS, such as any one of exons 1, 2, 3, 4, 5, 6, or 7. In some embodiments, the nucleic acid encoding a selection agent or a detection agent is integrated at an intron of TYMS, such as any one of introns 1, 2, 3, 4, 5, or 6. In some embodiments, the nucleic acid encoding the selection agent or the detection agent is integrated in the 5′ UTR of TYMS. In some embodiments, the nucleic acid encoding the selection agent or the detection agent is integrated in the 3′ UTR of TYMS.

In some embodiments, the endogenous proliferation gene is H2AZ1. In some embodiments, the nucleic acid encoding a selection agent or a detection agent is integrated at an exon of H2AZ1, such as any one of exons 1, 2, 3, 4, or 5. In some embodiments, the nucleic acid encoding a selection agent or a detection agent is integrated at an intron of H2AZ1, such as any one of introns 1, 2, 3, or 4. In some embodiments, the nucleic acid encoding the selection agent or the detection agent is integrated in the 5′ UTR of H2AZ1. In some embodiments, the nucleic acid encoding the selection agent or the detection agent is integrated in the 3′ UTR of H2AZ1.

In some embodiments, the endogenous proliferation gene is TMSB15A. In some embodiments, the nucleic acid encoding a selection agent or a detection agent is integrated at an exon of TMSB15A, such as any one of exons 1, 2, or 3. In some embodiments, the nucleic acid encoding a selection agent or a detection agent is integrated at an intron of TMSB15A, such as any one of introns 1 or 2. In some embodiments, the nucleic acid encoding the selection agent or the detection agent is integrated in the 5′ UTR of TMSB15A. In some embodiments, the nucleic acid encoding the selection agent or the detection agent is integrated in the 3′ UTR of TMSB15A.

In some embodiments, the endogenous proliferation gene is CENPF. In some embodiments, the nucleic acid encoding a selection agent or a detection agent is integrated at an exon of CENPF, such as any one of exons 1, 2, 3, 4, 5, 6, 7, 8, 9, 10, 11, 12, 13, 14, 15, 16, 17, 18, 19, or 20. In some embodiments, the nucleic acid encoding a selection agent or a detection agent is integrated at an intron of CENPF, such as any one of introns 1, 2, 3, 4, 5, 6, 7, 8, 9, 10, 11, 12, 13, 14, 15, 16, 17, 18, or 19. In some embodiments, the nucleic acid encoding the selection agent or the detection agent is integrated in the 5′ UTR of CENPF. In some embodiments, the nucleic acid encoding the selection agent or the detection agent is integrated in the 3′ UTR of CENPF.

In some embodiments, the endogenous proliferation gene is MKI67. In some embodiments, the nucleic acid encoding a selection agent or a detection agent is integrated at an exon of MKI67, such as any one of exons 1, 2, 3, 4, 5, 6, 7, 8, 9, 10, 11, 12, 13, 14, or 15. In some embodiments, the nucleic acid encoding a selection agent or a detection agent is integrated at an intron of MKI67, such as any one of introns 1, 2, 3, 4, 5, 6, 7, 8, 9, 10, 11, 12, 13, or 14. In some embodiments, the nucleic acid encoding the selection agent or the detection agent is integrated in the 5′ UTR of MKI67. In some embodiments, the nucleic acid encoding the selection agent or the detection agent is integrated in the 3′ UTR of MKI67.

In some embodiments, the modified cell described herein has at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% gene expression at the modified locus relative to the unmodified locus. In some embodiments, the modified cell described herein has 100% gene expression at the modified locus relative to that of the unmodified locus. In some embodiments, the modified cell described herein has a growth rate equal to or faster than that of unmodified cells.

### B. Off-Target Cell Marker Genes

Cell types in the body exhibit diverse properties in many modalities, including molecular, morphological, physiological, and functional. Cellular properties at the individual cell level are highly heterogeneous. Variations in different modalities do not necessarily exhibit high degrees of unity. However, single-cell transcriptomics has allowed robust cell type classification. Molecular approaches for profiling a cell type using single-cell or single-nucleus RNA-sequencing are used to generate comprehensive atlases of cell types and their markers. Off-target cell marker genes are genes that are highly expressed in one cell type, but are lowly expressed in other types. Off-target cell marker genes define cellular identity. The off-target cell marker gene may be specific to sample type and/or species. Off-target cell marker genes provide insights into the core set of genes whose expression is shared among all cell of a given type and have been used to annotate cell clusters and to examine the cellular composition of bulk tissues. Measurement of specific off-target cell marker genes allows for the identification and verification of the desired differentiated cell type needed for gene or cell therapy.

In some embodiments, there is provided a modified cell comprising a nucleic acid encoding a selection agent or a detection agent integrated at an off-target cell type gene locus and operably linked to the promoter of the off-target cell marker gene, wherein expression of the nucleic acid encoding the selection agent or the detection agent is regulated by the promoter of the off-target cell marker gene and/or regulatory elements of the off-target cell marker gene. In some embodiments, there is provided a modified cell comprising a nucleic acid encoding a selection agent and a detection agent integrated at an off-target cell type gene locus and operably linked to the promoter of the off-target cell type gene, wherein expression of the nucleic acid encoding the selection agent and the detection agent is regulated by the promoter of the off-target cell type gene and/or regulatory elements of the off-target cell type gene. In some embodiments, there is provided a modified cell comprising a nucleic acid encoding a first detection agent and a second detection agent, wherein the nucleic acid is integrated at an off-target cell type gene locus and operably linked to the promoter of the off-target cell type gene, and wherein expression of the nucleic acid encoding the first detection agent and the second detection agent is regulated by the promoter of the off-target cell type gene and/or regulatory elements of the off-target cell type gene. In some embodiments, there is provided a modified cell comprising a nucleic acid encoding a first selection agent and a second selection agent, wherein the nucleic acid is integrated at an off-target cell type gene locus and operably linked to the promoter of the off-target cell type gene, and wherein expression of the nucleic acid encoding the first selection agent and the second selection agent is regulated by the promoter of the off-target cell type gene and/or regulatory elements of the off-target cell type gene. In some embodiments, the first selection agent and the second selection agent are operably linked. In some embodiments, the first selection agent and the second selection agent are operably linked by an internal ribosome entry site. In some embodiments, the first selection agent and the second selection agent are operably linked by a self-cleaving peptide. In some embodiments, there is provided a modified cell comprising a first nucleic acid encoding a selection agent and a second nucleic acid encoding a detection agent, wherein the first nucleic acid is integrated at a first off-target cell type gene locus and operably linked to the promoter of the first off-target cell type gene, wherein expression of the first nucleic acid encoding the selection agent is regulated by the promoter of the first off-target cell type gene and/or regulatory elements of the first off-target cell type gene, wherein the second nucleic acid is integrated at a second off-target cell type gene locus and operably linked to the promoter of the second off-target cell type gene, and wherein expression of the second nucleic acid encoding the detection agent is regulated by the promoter of the second off-target cell type gene and/or regulatory elements of the off-target cell type gene. In some embodiments, there is provided a modified cell comprising a first nucleic acid encoding a first selection agent and a second nucleic acid encoding a second selection agent, wherein the first nucleic acid is integrated at a first off-target cell type gene locus and operably linked to the promoter of the first off-target cell type gene, wherein expression of the first nucleic acid encoding the first selection agent is regulated by the promoter of the first off-target cell type gene and/or regulatory elements of the first off-target cell type gene, wherein the second nucleic acid is integrated at a second off-target cell type gene locus and operably linked to the promoter of the second off-target cell type gene, and wherein expression of the second nucleic acid encoding the second selection agent is regulated by the promoter of the second off-target cell type gene and/or regulatory elements of the off-target cell type gene. In some embodiments, there is provided a modified cell comprising a first nucleic acid encoding a first detection agent and a second nucleic acid encoding a second detection agent, wherein the first nucleic acid is integrated at a first off-target cell type gene locus and operably linked to the promoter of the first off-target cell type gene, wherein expression of the first nucleic acid encoding the first detection agent is regulated by the promoter of the first off-target cell type gene and/or regulatory elements of the first off-target cell type gene, wherein the second nucleic acid is integrated at a second off-target cell type gene locus and operably linked to the promoter of the off-target cell type gene, and wherein expression of the second nucleic acid encoding the second detection agent is regulated by the promoter of the second off-target cell type gene and/or regulatory elements of the second off-target cell type gene. In some embodiments, the first endogenous proliferation gene and/or the second endogenous proliferation gene are expressed in a cell that is included in a population of therapeutic cells. In some embodiments, the first endogenous proliferation gene and/or the second endogenous proliferation gene are expressed in a cell that is included in a population of therapeutic cells but is not a therapeutic cell. In some embodiments, the cell is an off-target cell. In some embodiments, the first off-target cell type gene and the second off-target cell type gene are the same, and the first and second nucleic acids are integrated at different alleles. In some embodiments, first off-target cell type gene and the second off-target cell type gene are different. In some embodiments, the nucleic acid encoding the selection agent or the detection agent is integrated in an exon or an intron of the off-target cell marker gene. In some embodiments, the nucleic acid encoding the selection agent or the detection agent is integrated in a 5′ UTR or a 3′ UTR of the off-target cell marker gene. In some embodiments, following integration, the off-target cell marker gene locus comprises i) nucleic acid encoding the off-target cell marker gene, ii) an internal ribosomal entry site or a self-cleaving RNA site, and iii) the nucleic acid encoding the selection agent or a detection agent. Examples of off-target cell marker genes include, but are not limited to, ANXA1, KRT19, CTSC, DSC2, ARHGAP29, KRT18, KRT8, CD9, PLK2, and KRT17.

In some embodiments, the off-target cell marker gene is ANXA1. In some embodiments, the nucleic acid encoding a selection agent or a detection agent is integrated at an exon of ANXA1, such as any one of exons 1, 2, 3, 4, 5, 6, 7, 8, 9, 10, 11, 12, or 13. In some embodiments, the nucleic acid encoding a selection agent or a detection agent is integrated at an intron of ANXA1, such as any one of introns 1, 2, 3, 4, 5, 6, 7, 8, 9, 10, 11, or 12. In some embodiments, the nucleic acid encoding the selection agent or the detection agent is integrated in the 5′ UTR of ANXA1. In some embodiments, the nucleic acid encoding the selection agent or the detection agent is integrated in the 3′ UTR of ANXA1.

In some embodiments, the off-target cell marker gene is KRT19. In some embodiments, the nucleic acid encoding a selection agent or a detection agent is integrated at an exon of KRT19, such as any one of exons 1, 2, 3, 4, 5, or 6. In some embodiments, the nucleic acid encoding a selection agent or a detection agent is integrated at an intron of KRT19, such as any one of introns 1, 2, 3, 4, or 5. In some embodiments, the nucleic acid encoding the selection agent or the detection agent is integrated in the 5′ UTR of KRT19. In some embodiments, the nucleic acid encoding the selection agent or the detection agent is integrated in the 3′ UTR of KRT19.

In some embodiments, the off-target cell marker gene is CTSC. In some embodiments, the nucleic acid encoding a selection agent or a detection agent is integrated at an exon of CTSC, such as any one of exons 1, 2, 3, 4, 5, 6, or 7. In some embodiments, the nucleic acid encoding a selection agent or a detection agent is integrated at an intron of CTSC, such as any one of introns 1, 2, 3, 4, 5, or 6. In some embodiments, the nucleic acid encoding the selection agent or the detection agent is integrated in the 5′ UTR of CTSC. In some embodiments, the nucleic acid encoding the selection agent or the detection agent is integrated in the 3′ UTR of CTSC.

In some embodiments, the off-target cell marker gene is DSC2. In some embodiments, the nucleic acid encoding a selection agent or a detection agent is integrated at an exon of DSC2, such as any one of exons 1, 2, 3, 4, 5, 6, 7, 8, 9, 10, 11, 12, 13, 14, 15, or 16. In some embodiments, the nucleic acid encoding a selection agent or a detection agent is integrated at an intron of DSC2, such as any one of introns 1, 2, 3, 4, 5, 6, 7, 8, 9, 10, 11, 12, 13, 14, or 15. In some embodiments, the nucleic acid encoding the selection agent or the detection agent is integrated in the 5′ UTR of DSC2. In some embodiments, the nucleic acid encoding the selection agent or the detection agent is integrated in the 3′ UTR of DSC2.

In some embodiments, the off-target cell marker gene is ARHGAP29. In some embodiments, the nucleic acid encoding a selection agent or a detection agent is integrated at an exon of ARHGAP29, such as any one of exons 1, 2, 3, 4, 5, 6, 7, 8, 9, 10, 11, 12, 13, 14, 15, or 16. In some embodiments, the nucleic acid encoding a selection agent or a detection agent is integrated at an intron of ARHGAP29, such as any one of introns 1, 2, 3, 4, 5, 6, 7, 8, 9, 10, 11, 12, 13, 14, or 15. In some embodiments, the nucleic acid encoding the selection agent or the detection agent is integrated in the 5′ UTR of ARHGAP29. In some embodiments, the nucleic acid encoding the selection agent or the detection agent is integrated in the 3′ UTR of ARHGAP29.

In some embodiments, the off-target cell marker gene is KRT18. In some embodiments, the nucleic acid encoding a selection agent or a detection agent is integrated at an exon of KRT18, such as any one of exons 1, 2, 3, 4, 5, 6, or 7. In some embodiments, the nucleic acid encoding a selection agent or a detection agent is integrated at an intron of KRT18, such as any one of introns 1, 2, 3, 4, 5, or 6. In some embodiments, the nucleic acid encoding the selection agent or the detection agent is integrated in the 5′ UTR of KRT18. In some embodiments, the nucleic acid encoding the selection agent or the detection agent is integrated in the 3′ UTR of KRT18.

In some embodiments, the off-target cell marker gene is KRT8. In some embodiments, the nucleic acid encoding a selection agent or a detection agent is integrated at an exon of KRT8, such as any one of exons 1, 2, 3, 4, 5, 6, 7, or 8. In some embodiments, the nucleic acid encoding a selection agent or a detection agent is integrated at an intron of KRT8, such as any one of introns 1, 2, 3, 4, 5, 6, or 7. In some embodiments, the nucleic acid encoding the selection agent or the detection agent is integrated in the 5′ UTR of KRT8. In some embodiments, the nucleic acid encoding the selection agent or the detection agent is integrated in the 3′ UTR of KRT8.

In some embodiments, the off-target cell marker gene is CD9. In some embodiments, the nucleic acid encoding a selection agent or a detection agent is integrated at an exon of CD9, such as any one of exons 1, 2, 3, 4, 5, 6, 7, or 8. In some embodiments, the nucleic acid encoding a selection agent or a detection agent is integrated at an intron of CD9, such as any one of introns 1, 2, 3, 4, 5, 6, or 7. In some embodiments, the nucleic acid encoding the selection agent or the detection agent is integrated in the 5′ UTR of CD9. In some embodiments, the nucleic acid encoding the selection agent or the detection agent is integrated in the 3′ UTR of CD9.

In some embodiments, the off-target cell marker gene is PLK2. In some embodiments, the nucleic acid encoding a selection agent or a detection agent is integrated at an exon of PLK2, such as any one of exons 1, 2, 3, 4, 5, 6, 7, 8, 9, 10, 11, 12, 13, or 14. In some embodiments, the nucleic acid encoding a selection agent or a detection agent is integrated at an intron of PLK2, such as any one of introns 1, 2, 3, 4, 5, 6, 7, 8, 9, 10, 11, 12, or 13. In some embodiments, the nucleic acid encoding the selection agent or the detection agent is integrated in the 5′ UTR of PLK2. In some embodiments, the nucleic acid encoding the selection agent or the detection agent is integrated in the 3′ UTR of PLK2.

In some embodiments, the off-target cell marker gene is KRT17. In some embodiments, the nucleic acid encoding a selection agent or a detection agent is integrated at an exon of KRT17, such as any one of exons 1, 2, 3, 4, 5, 6, 7, or 8. In some embodiments, the nucleic acid encoding a selection agent or a detection agent is integrated at an intron of KRT17, such as any one of introns 1, 2, 3, 4, 5, 6, or 7. In some embodiments, the nucleic acid encoding the selection agent or the detection agent is integrated in the 5′ UTR of KRT17. In some embodiments, the nucleic acid encoding the selection agent or the detection agent is integrated in the 3′ UTR of KRT17.

As used herein, an “IC50” is used to indicate the effective concentration of an inducer or activator needed to achieve 50% cell viability in vitro. IC₅₀ can be measured by bioassays such as a dosing kill curve. The values are typically expressed as molar concentration. In some embodiments, the gene candidate was chosen based on the IC₅₀ in a dosing kill curve assay. In some embodiments, the IC₅₀ value of the inducer in causing 50% cell death at the iPSC stage is less than about 50 μM, about 49 μM, about 48 μM, about 47 μM, about 46 μM, about 45 μM, about 44 μM, about 43 μM, about 42 μM, about 41 μM, about 40 μM, about 39 μM, about 38 μM, about 37 μM, about 36 μM, about 35 μM, about 34 μM, about 33 μM, about 32 μM, about 31 μM, about 30 μM, about 29 μM, about 28 μM, about 27 μM, about 26 μM, about 25 μM, about 24 μM, about 23 μM, about 22 μM, about 21 μM, about 20 μM, about 19 μM, about 18 μM, about 17 μM, about 16 μM, about 15 μM, about 14 μM, about 13 μM, about 12 μM, about 11 μM, about 10 μM, about 9 μM, about 8 μM, about 7 μM, about 6 μM, about 5 μM, about 4 μM, about 3 μM, about 2 μM, or about 1 μM.

In some embodiments, the gene candidate was chosen based on the percentage of viable cells in the non-proliferative state (e.g., day 18 of differentiation). In some embodiments, the percentage of resistant cells in the non-proliferative state (e.g., day 18 of differentiation) is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

### C. Exogenous Sequences

The cells of the present disclosure may be modified to include any exogenous nucleic acid sequence of interest at an endogenous proliferation gene or an off-target cell marker gene, e.g., as described above. In some embodiments, the exogenous sequence(s) inserted at an endogenous proliferation gene or an off-target cell marker gene comprise of selection agents, detection agents, genome editing complexes, and combinations thereof, as described in greater detail below.

### (1) Transgenes

i. Kill Switches

In some embodiments, the one or more transgenes comprise a kill switch. In some embodiments, the selection agent is a kill switch. A “kill switch” can cause the death of the cell, such as after the modified cell is administered to a subject and if the cells should grow and divide in an undesired manner. A kill switch can be activated by a specific compound. The result is specifically eliminating cells expressing a specific gene (e.g., an endogenous proliferation gene or an off-target cell marker gene). Inclusion of a kill switch allows for controlled killing of the cells in the event of cytotoxicity or other negative consequences to the recipient, thus increasing the safety of cell-based therapies.

In some embodiments, a kill switch can be incorporated into, such as introduced, into a modified cell provided herein to provide the ability to induce death or apoptosis of modified cell containing the kill switch, for example if the cells grow and divide in an undesired manner or cause excessive toxicity to the host. Thus, the use of kill switches enables one to conditionally eliminate aberrant cells in vivo and can be a critical step for the application of cell therapies in the clinic. Kill switches and their uses thereof are described in, for example, Duzgune§, Origins of Suicide Gene Therapy (2019); Duzgune§ (eds), Suicide Gene Therapy. Methods in Molecular Biology, vol. 1895 (Humana Press, New York, NY) (for HSV-tk, cytosine deaminase, nitroreductase, purine nucleoside phosphorylase, and horseradish peroxidase); Zhou and Brenner, Exp Hematol 44(11):1013-1019 (2016) (for iCaspase9); Wang et al., Blood 18(5):1255-1263 (2001) (for huEGFR); U.S. Patent Application Publication No. 20180002397 (for HER1); and Philip et al., Blood124(8):1277-1287 (2014) (for RQR8).

In some embodiments, the kill switch can cause cell death in a controlled manner, for example, in the presence of a drug or prodrug or upon activation by a selective exogenous compound. In some embodiments, the kill switch is selected from the group consisting of herpes simplex virus thymidine kinase (HSV-tk), cytosine deaminase (CyD), nitroreductase (NTR), purine nucleoside phosphorylase (PNP), horseradish peroxidase, inducible caspase 9 (iCasp9), rapamycin-activated caspase 9 (rapaCasp9), chemically regulated-SH2-delivered inhibitory tail (CRASH-IT), CCR4, CD16, CD19, CD20, CD30, EGFR, GD2, HER1, HER2, MUC1, PSMA, and RQR8.

In some embodiments, the kill switch may be a transgene encoding a product with cell killing capabilities when activated by a drug or prodrug, for example, by turning a non-toxic prodrug to a toxic metabolite inside the cell. In these embodiments, cell killing is activated by contacting a modified cell with the drug or prodrug. In some cases, the kill switch is HSV-tk, which converts ganciclovir (GCV) to GCV-triphosphate, thereby interfering with DNA synthesis and killing dividing cells. In some cases, the kill switch is CyD or a variant thereof, which converts the antifungal drug 5-fluorocytosine (5-FC) to cytotoxic 5-fluorouracil (5-FU) by catalyzing the hydrolytic deamination of cytosine into uracil. 5-FU is further converted to potent anti-metabolites (5-FdUMP, 5-FdUTP, 5-FUTP) by cellular enzymes. These compounds inhibit thymidylate synthase and the production of RNA and DNA, resulting in cell death. In some cases, the kill switch is NTR or a variant thereof, which can act on the prodrug CB 1954 via reduction of the nitro groups to reactive N-hydroxylamine intermediates that are toxic in proliferating and nonproliferating cells. In some cases, the kill switch is PNP or a variant thereof, which can turn prodrug 6-methylpurine deoxyriboside or fludarabine into toxic metabolites to both proliferating and nonproliferating cells. In some cases, the kill switch is horseradish peroxidase or a variant thereof, which can catalyze indole-3-acetic acid (IAA) to a potent cytotoxin and thus achieve cell killing. In other embodiments, the suicide gene is a cytosine deaminase (e.g., the Escherichia coli cytosine deaminase (EC-CD)) gene, and the trigger is 5-fluorocytosine (5-FC) (Barese et al., Mol. Therap. 20(10): 1932-1943 (2012) and Xu et al., Cell Res. 8:73-8 (1998), both incorporated herein by reference in their entirety). In some embodiments, the kill switch and genes associated with the kill switch are expressed from a bicistronic cassette. In some embodiments, the kill switch and one or more tolerogenic factors are expressed from a bicistronic cassette.

In some embodiments, the kill switch may be an iCasp9. Caspase 9 is a component of the intrinsic mitochondrial apoptotic pathway which, under physiological conditions, is activated by the release of cytochrome C from damaged mitochondria. Activated caspase 9 then activates caspase 3, which triggers terminal effector molecules leading to apoptosis. The iCasp9 may be generated by fusing a truncated caspase 9 (without its physiological dimerization domain or caspase activation domain) to a FK506 binding protein (FKBP), FKBP12-F36V, via a peptide linker. The iCasp9 has low dimer-independent basal activity and can be stably expressed in host cells (e.g., human T cells) without impairing their phenotype, function, or antigen specificity. However, in the presence of chemical inducer of dimerization (CID), such as rimiducid (AP1903), AP20187, and rapamycin, iCasp9 can undergo inducible dimerization and activate the downstream caspase molecules, resulting in apoptosis of cells expressing the iCasp9. See, e.g., PCT Application Publication No. WO2011/146862; Stasi et al., N. Engl. J. Med. 365; 18 (2011); Tey et al., Biol. Blood Marrow Transplant 13:913-924 (2007). In particular, the rapamycin-inducible caspase 9 variant is called rapaCasp9. See Stavrou et al., Mal. Ther. 26(5):1266-1276 (2018). Thus, iCasp9 can be used as a kill switch to achieve controlled killing of the host cells.

In some embodiments, the kill switch may be a membrane-expressed protein, which allows for cell depletion after administration of a specific antibody to that protein. Kill switches of this category may include, for example, one or more transgene encoding CCR4, CD16, CD19, CD20, CD30, EGFR, GD2, HER1, HER2, MUC1, PSMA, or RQR8 for surface expression thereof. These proteins may have surface epitopes that can be targeted by specific antibodies. In some embodiments, the kill switch comprises CCR4, which can be recognized by an anti-CCR4 antibody. Non-limiting examples of suitable anti-CCR4 antibodies include mogamulizumab and biosimilars thereof. In some embodiments, the kill switch comprises CD16 or CD30, which can be recognized by an anti-CD16 or anti-CD30 antibody. Non-limiting examples of such antiCD16 or anti-CD30 antibody include AFM13 and biosimilars thereof. In some embodiments, the kill switch comprises CD19, which can be recognized by an anti-CD19 antibody. Non-limiting examples of such anti-CD19 antibody include MOR208 and biosimilars thereof. In some embodiments, the kill switch comprises CD20, which can be recognized by an anti-CD20 antibody. Non-limiting examples of such anti-CD20 antibody include obinutuzumab, ublituximab, ocaratuzumab, rituximab, rituximab-R11b, and biosimilars thereof. Cells that express the kill switch are thus CD20-positive and can be targeted for killing through administration of an anti-CD20 antibody as described. In some embodiments, the kill switch comprises EGFR, which can be recognized by an anti-EGFR antibody. Non-limiting examples of such anti-EGFR antibody include tomuzotuximab, R05083945 (GA201), cetuximab, and biosimilars thereof. In some embodiments, the kill switch comprises GD2, which can be recognized by an anti-GD2 antibody. Non-limiting examples of such anti-GD2 antibody include Hu14.18K322A, Hu14.18-IL2, Hu3F8, dinituximab, c.60C3-R11c, and biosimilars thereof.

In some embodiments, the expression of a detection agent acts as a signal for the administration of an antibody directed against or specific to a tolerogenic agent, e.g., an anti-CD47 antibody. In some embodiments, the kill switch may be an exogenously administered agent that recognizes one or more tolerogenic factor on the surface of the modified cells. In some embodiments, the exogenously administered agent is an antibody directed against or specific to a tolerogenic agent, e.g., an anti-CD47 antibody. By recognizing and blocking a tolerogenic factor on modified cells, an exogenously administered antibody may block the immune inhibitory functions of the tolerogenic factor thereby re-sensitizing the immune system to the modified cells. For instance, for modified cells that overexpresses CD47, an exogenously administered anti-CD47 antibody may be administered to the subject, resulting in masking of CD47 on the modified cells and triggering of an immune response to the modified cells. In some embodiments, the anti-CD47 antibody is Magrolimab.

In some the safety switch comprises an anti-CD47 antibody. In some embodiments, the anti-CD47 antibody is Magrolimab. In some embodiments, the safety switch is Magrolimab.

In some embodiments, the method further comprises introducing an expression vector comprising an inducible suicide switch into the cell.

In some embodiments, the tolerogenic factor is CD47 and the cell includes an exogenous polynucleotide encoding a CD47 protein. In some embodiments, the cell expresses an exogenous CD47 polypeptide.

In some embodiments, a method disclosed herein comprises administering to a subject in need thereof a CD47-SIRPα blockade agent, wherein the subject was previously administered modified cells engineered to express an exogenous CD47 polypeptide. In some embodiments, the CD47-SIRPα blockade agent comprises a CD47-binding domain. In some embodiments, the CD47-binding domain comprises signal regulatory protein alpha (SIRPα) or a fragment thereof. In some embodiments, the CD47-SIRPα blockade agent comprises an immunoglobulin G (IgG) Fc domain. In some embodiments, the IgG Fc domain comprises an IgG1 Fc domain. In some embodiments, the IgG1 Fc domain comprises a fragment of a human antibody. In some embodiments, the CD47-SIRPα blockade agent is selected from the group consisting of TTI-621, TTI-622, and ALX148. In some embodiments, the CD47-SIRPα blockade agent is TTI-621, TTI-622, and ALX148. In some embodiments, the CD47-SIRPα blockade agent is TTI-622. In some embodiments, the CD47-SIRPα blockade agent is ALX148. In some embodiments, the IgG Fc domain comprises an IgG4 Fc domain. In some embodiments, the CD47-SIRPα blockade agent is an antibody. In some embodiments, the antibody is selected from the group consisting of MIAP410, B6H12, and Magrolimab. In some embodiments, the antibody is MIAP410. In some embodiments, the antibody is B6H12. In some embodiments, the antibody is Magrolimab. In some embodiments, the antibody is selected from the group consisting of AO-176, IBI188 (letaplimab), STI-6643, and ZL-1201. In some embodiments, the antibody is AO-176 (Arch). In some embodiments, the antibody is IBI188 (letaplimab) (Innovent). In some embodiments, the antibody is STI-6643 (Sorrento). In some embodiments, the antibody is ZL-1201 (Zai).

In some embodiments, useful antibodies or fragments thereof that bind CD47 can be selected from a group that includes magrolimab ((Hu5F9-G4)) (Forty Seven, Inc.; Gilead Sciences, Inc.), urabrelimab, CC-90002 (Celgene; Bristol-Myers Squibb), IBI-188 (Innovent Biologics), IBI-322 (Innovent Biologics), TG-1801 (TG Therapeutics; also known as NI-1701, Novimmune SA), ALX148 (ALX Oncology), TJ011133 (also known as TJC4, I-Mab Biopharma), FA3M3, ZL-1201 (Zai Lab Co., Ltd), AK117 (Akesbio Australia Pty, Ltd.), AO-176 (Arch Oncology), SRF231 (Surface Oncology), GenSci-059 (GeneScience), C47B157 (Janssen Research and Development), C47B161 (Janssen Research and Development), C47B167 (Janssen Research and Development), C47B222 (Janssen Research and Development), C47B227 (Janssen Research and Development), Vx-1004 (Corvus Pharmaceuticals), HMBD004 (Hummingbird Bioscience Pte Ltd), SHR-1603 (Hengrui), AMMS4-G4 (Beijing Institute of Biotechnology), RTX-CD47 (University of Groningen), and IMC-002. (Samsung Biologics; ImmuneOncia Therapeutics). In some embodiments, the antibody or fragment thereof does not compete for CD47 binding with an antibody selected from a group that includes magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof competes for CD47 binding with an antibody selected from magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof that binds CD47 is selected from a group that includes a single-chain Fv fragment (scFv) against CD47, a Fab against CD47, a VHH nanobody against CD47, a DARPin against CD47, and variants thereof. In some embodiments, the scFv against CD47, a Fab against CD47, and variants thereof are based on the antigen binding domains of any of the antibodies selected from a group that includes magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002.

In some embodiments, the CD47 antagonist provides CD47 blockade. Methods and agents for CD47 blockade are described in PCT/US2021/054326, which is incorporated by reference in its entirety.

In some embodiments, the modified cells are derived from a source cell already comprising one or more of the desired modifications. In some embodiments, in view of the teachings provided herein one of ordinary skill in the art will readily appreciate how to assess what modifications are required to arrive at the desired final form of a modified cell and that not all reduced or increased levels of target components are achieved via active engineering. In some embodiments, the modifications of the modified cells may be in any order, and not necessarily the order listed in the descriptive language provided herein.

Once altered, the presence of expression of any of the molecule described herein can be assayed using known techniques, such as Western blots, ELISA assays, FACS assays, flow cytometry, and the like.

In some embodiments, the kill switch can include any of the strategies as described in WO2021146627A1, which is incorporated by reference in its entirety.

In some embodiments, a kill switch may be introduced into a modified cell of the disclosure as part of an expression vector comprising, e.g., an inducible kill switch.

In some embodiments, there is provided a cell comprising a nucleic acid encoding a selection agent or a detection agent integrated at an endogenous proliferation gene locus and operably linked to the promoter of the endogenous proliferation gene, wherein expression of the nucleic acid encoding the selection agent or the detection agent is regulated by the promoter of the endogenous proliferation gene and/or regulatory elements of the endogenous proliferation gene. In some embodiments, the endogenous proliferation gene is selected from the group consisting of AURKB, CDK1, CDC20, RRM2, BIRC5, TOP2A, PTTG1, CCNB1, TPX2, KIF11, SPC25, CENPK, SMC4, TYMS, H2AZ1, TMSB15A, CENPF, and MKI67. In some embodiments, the selection agent is iCaspase 9 or cytosine deaminase. In some embodiments, (i) the endogenous proliferation gene is AURKB and the selection agent is iCaspase9; (ii) the endogenous proliferation gene is CDK1 and the selection agent is iCaspase9; (iii) the endogenous proliferation gene is CDC20 and the selection agent is iCaspase9; (iv) the endogenous proliferation gene is RRM2 and the selection agent is iCaspase9; (v) the endogenous proliferation gene is BIRC5 and the selection agent is iCaspase9; (vi) the endogenous proliferation gene is TOP2A and the selection agent is iCaspase9; (vii) the endogenous proliferation gene is PTTG1 and the selection agent is iCaspase9; (viii) the endogenous proliferation gene is CCNB1 and the selection agent is iCaspase9; (ix) the endogenous proliferation gene is TPX2 and the selection agent is iCaspase9; (x) the endogenous proliferation gene is KIF11 and the selection agent is iCaspase9; (xi) the endogenous proliferation gene is SPC25 and the selection agent is iCaspase9; (xii) the endogenous proliferation gene is CENPK and the selection agent is iCaspase9; (xiii) the endogenous proliferation gene is SMC4 and the selection agent is iCaspase9; (xiv) the endogenous proliferation gene is TYMS and the selection agent is iCaspase9; (xv) the endogenous proliferation gene is H2AZ1 and the selection agent is iCaspase9; (xvi) the endogenous proliferation gene is TMSB15A and the selection agent is iCaspase9; (xvii) the endogenous proliferation gene is CENPF and the selection agent is iCaspase9; (xviii) the endogenous proliferation gene is MKI67 and the selection agent is iCaspase9; (xix) the endogenous proliferation gene is AURKB and the selection agent is cytosine deaminase; (xx) the endogenous proliferation gene is CDK1 and the selection agent is cytosine deaminase; (xxi) the endogenous proliferation gene is CDC20 and the selection agent is cytosine deaminase; (xxii) the endogenous proliferation gene is RRM2 and the selection agent is cytosine deaminase; (xxiii) the endogenous proliferation gene is BIRC5 and the selection agent is cytosine deaminase; (xxiv) the endogenous proliferation gene is TOP2A and the selection agent is cytosine deaminase; (xxv) the endogenous proliferation gene is PTTG1 and the selection agent is cytosine deaminase; (xxvi) the endogenous proliferation gene is CCNB1 and the selection agent is cytosine deaminase; (xxvii) the endogenous proliferation gene is TPX2 and the selection agent is cytosine deaminase; (xxviii) the endogenous proliferation gene is KIF11 and the selection agent is cytosine deaminase; (xxix) the endogenous proliferation gene is SPC25 and the selection agent is cytosine deaminase; (xxx) the endogenous proliferation gene is CENPK and the selection agent is cytosine deaminase; (xxxi) the endogenous proliferation gene is SMC4 and the selection agent is cytosine deaminase; (xxxii) the endogenous proliferation gene is TYMS and the selection agent is cytosine deaminase; (xxxiii) the endogenous proliferation gene is H2AZ1 and the selection agent is cytosine deaminase; (xxxiv) the endogenous proliferation gene is TMSB15A and the selection agent is cytosine deaminase; (xxxv) the endogenous proliferation gene is CENPF and the selection agent is cytosine deaminase; or (xxxvi) the endogenous proliferation gene is MKI67 and the selection agent is cytosine deaminase.

In some embodiments, the selection agent or the detection agent of the cell described herein is expressed in proliferating cells. In some embodiments, the selection agent or the detection agent of the cell described herein is not expressed in non-proliferating cells. In some embodiments, the selection agent or the detection agent of the cell described herein is expressed in partially differentiated cells. In some embodiments, the selection agent or the detection agent of the cell described herein is expressed in an intermediate cell type made during differentiation. In some embodiments, the selection agent or the detection agent of the cell described herein is not expressed in differentiated cells. In some embodiments, the selection agent or the detection agent of the cell described herein is not expressed in a therapeutic cell. In some embodiments, the cell is selected from the group consisting of a pancreatic islet cell, an alpha cell, a beta cell, a gamma cell, a delta cell, an epsilon cell, a T cell, a neuron, a glial cell, a cardiomyocyte, a retinal pigmented epithelial cell, a hematopoietic progenitor cell, a natural killer cell, an endothelial cell, and a lung cell.

In some embodiments, there is provided a cell comprising a nucleic acid encoding a selection agent or a detection agent integrated at an off-target cell type gene locus and operably linked to the promoter of the off-target cell marker gene, wherein expression of the nucleic acid encoding the selection agent or the detection agent is regulated by the promoter of the off-target cell marker gene and/or regulatory elements of the off-target cell marker gene. In some embodiments, the off-target cell marker gene is selected from the group consisting of a pluripotency cell marker gene, a tumorigenic cell marker gene, a ductal marker gene, an enterochromaffin marker gene, a neural marker gene, acinar marker gene, intestinal marker gene, endothelial marker gene, mesenchymal fibroblast marker gene, muscle marker gene, osteoblast marker gene, and stromal marker gene. In some embodiments, the off-target cell marker gene is selected from the group consisting of ANXA1, KRT19, CTSC, DSC2, ARHGAP29, KRT18, KRT8, CD9, PLK2, and KRT17. In some embodiments, the selection agent is iCaspase 9 or cytosine deaminase. In some embodiments, (i) the off-target cell marker gene is ANXA1 and the selection agent is iCaspase9; (ii) the off-target cell marker gene is KRT19 and the selection agent is iCaspase9; (iii) the off-target cell marker gene is CTSC and the selection agent is iCaspase9; (iv) the off-target cell marker gene is DSC2 and the selection agent is iCaspase9; (v) the off-target cell marker gene is ARHGAP29 and the selection agent is iCaspase9; (vi) the off-target cell marker gene is KRT18 and the selection agent is iCaspase9; (vii) the off-target cell marker gene is KRT8 and the selection agent is iCaspase9; (viii) the off-target cell marker gene is CD9 and the selection agent is iCaspase9; (ix) the off-target cell marker gene is PLK2 and the selection agent is iCaspase9; (x) the off-target cell marker gene is KRT17 and the selection agent is iCaspase9; (xi) the off-target cell marker gene is ANXA1 and the selection agent is cytosine deaminase; (xii) the off-target cell marker gene is KRT19 and the selection agent is cytosine deaminase; (xiii) the off-target cell marker gene is CTSC and the selection agent is cytosine deaminase; (xiv) the off-target cell marker gene is DSC2 and the selection agent is cytosine deaminase; (xv) the off-target cell marker gene is ARHGAP29 and the selection agent is cytosine deaminase; (xvi) the off-target cell marker gene is KRT18 and the selection agent is cytosine deaminase; (xvii) the off-target cell marker gene is KRT8 and the selection agent is cytosine deaminase; (xviii) the off-target cell marker gene is CD9 and the selection agent is cytosine deaminase; (xix) the off-target cell marker gene is PLK2 and the selection agent is cytosine deaminase; or (xx) the off-target cell marker gene is KRT17 and the selection agent is cytosine deaminase.

In some embodiments, the selection agent or the detection agent of the cell described herein is expressed in a cell with a high level of expression of an off-target cell marker gene. In some embodiments, the selection agent or the detection agent is expressed in a cell with a high level of expression. In some embodiments, the selection agent or the detection agent is expressed in a cell with greater than about 3 transcripts per million (TPM), about 4 TPM, about 5 TPM, about 6 TPM, about 7 TPM, about 8 TPM, about 9 TPM, about 10 TPM, about 15 TPM, about 20 TPM, about 25 TPM, about 30 TPM, about 35 TPM, about 40 TPM, about 45 TPM, or about 50 TPM. In some embodiments, the selection agent or the detection agent is expressed in an off-target cell with greater than about 3 transcripts per million (TPM), about 4 TPM, about 5 TPM, about 6 TPM, about 7 TPM, about 8 TPM, about 9 TPM, about 10 TPM, about 15 TPM, about 20 TPM, about 25 TPM, about 30 TPM, about 35 TPM, about 40 TPM, about 45 TPM, or about 50 TPM. In some embodiments, the selection agent is expressed in a cell such that the selection agent enables selection of the cell. In some embodiments, the detection agent is expressed in a cell such that the detection agent enables detection of the cell.

In some embodiments, the selection agent or the detection agent of the cell described herein is expressed in a pluripotent cell. In some embodiments, the selection agent or the detection agent of the cell described herein is expressed in a tumorigenic cell. In some embodiments, the selection agent or the detection agent of the cell described herein is expressed in a ductal cell. In some embodiments, the selection agent or the detection agent of the cell described herein is expressed in an enterochromaffin cell. In some embodiments, the selection agent or the detection agent of the cell described herein is expressed in a neural cell. In some embodiments, the selection agent or the detection agent of the cell described herein is in partially differentiated cells. In some embodiments, the selection agent or the detection agent of the cell described herein is not expressed in differentiated cells. In some embodiments, the selection agent or the detection agent of the cell described herein is not expressed in a cell with a low level of expression or no expression of an off-target cell marker gene. In some embodiments, the selection agent or the detection agent of the cell described herein is not expressed in a therapeutic cell. In some embodiments, the off-target cell marker gene of the cell described herein is not a beta cell marker gene, a T cell marker gene, a glial cell marker gene, a cardiac cell marker gene, or a retinal pigment epithelium (RPE) cell marker gene.

ii. Genome-Editing Complexes

In some embodiments, the cell provided herein comprises a genome editing complex. Genome editing complexes are described in detail in Section C(2) below. In some embodiments, the one or more transgenes encode any of the genome editing complexes described in Section C(2) below.

In some specific embodiments, the genome editing complex comprises a genome targeting entity and/or a genome modifying entity. In some embodiments, the genome targeting entity is a nucleic acid-guided targeting entity, such as any of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising a gRNA and a Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof. In some embodiments, the genome targeting entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof. In some embodiments, the genome modifying entity comprises one or more of the following genome modifying activities: cleaving, deaminating, nicking, polymerizing, interrogating, integrating, cutting, unwinding, breaking, altering, methylating, demethylating, or otherwise destabilizing a target locus. In some embodiments, the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof. In some embodiments, the genome modifying entity is selected from a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof. In certain specific embodiments, the genome modifying entity is selected from Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor, a target-primed reverse transcription (TPRT) editor, APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof. In certain embodiments, the genome modifying entity is a Cas protein such as Cas9, or Mad7. In some embodiments, the genome targeting entity and the genome modifying entity are (a) different domains of a single polypeptide; (b) two different polypeptides that are operably linked together; or (c) two different polypeptides that are not linked together. In some embodiments, the genome editing complex comprises a guide nucleic acid having a targeting domain that is complementary to at least one target locus, optionally wherein the guide nucleic acid is a guide RNA (gRNA). In some cases, the genome editing complex is an RNA-guided nuclease, for example, a Cas nuclease and a guide RNA (CRISPR-Cas combination). In some embodiments, the CRISPR-Cas combination is a ribonucleoprotein (RNP) complex comprising the gRNA and the Cas nuclease. In some embodiments, the Cas nuclease is a Type II or Type V Cas protein. In some embodiments, the Cas nuclease is selected from Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, or Mad7.

iii. Barcodes

In some embodiments, the cell provided herein comprises a vector comprising a barcode. Barcodes generally refer to short, unique DNA sequences used to identify a target. Nucleic acid barcodes with unique, identifiable sequences can include (e.g.) molecular barcodes that can be used to tag, mark, delineate, identify, etc. a specific sequence, e.g., a transgene such that confirmation of the presence of the barcode is indicative of the presence of the associated transgene. Barcodes may be non-naturally occurring sequences. Barcodes may be used to identify cells or cell populations wherein the cells or cell populations express or contain a vector encoding a transgene sequence and the barcode, for example to differentiate engineered cells or populations of cells from wild-type cells. Barcodes are associated with specific transgenes such that each barcode is associated with one or more specific transgene, and identifying the presence of one or more barcodes within a cell or population of cells is indicative of the presence of the associated one or more transgenes. The diversity of barcodes allows for the pooling and simultaneous sequencing of cells, population of cells, and/or samples obtained from a patient using a high throughput, multiplex system, for example to screen the cells, population of cells, and/or samples obtained from a patient for the presence of a transgene by detecting the presence of the barcode. Accordingly, the barcode(s) of the present disclosure can be used to detect the presence of a transgene. See, e.g., U.S. 63/580,663, the contents of which are herein incorporated by reference.

In some embodiments, the barcode is located on a vector. In some embodiments, one or more barcodes are located on the same vector. In some embodiments, one or more barcodes are located on different vectors. In some embodiments, the barcode is flanked by primer binding sites. In some embodiments, the barcode is flanked by primer binding sites comprising a forward primer and a reverse primer.

In some embodiments, the first barcode and/or the second barcode is located outside of the first transgene and/or second transgene. In some embodiments, the first barcode and/or the second barcode is located within the first transgene and/or the second transgene. In some embodiments, a portion of the first barcode and/or the second barcode is located within the first transgene and/or the second transgene and a portion of the first barcode and/or the second barcode is located outside of the first transgene and/or second transgene.

In some embodiments, the first barcode and/or the second barcode comprises a diverged nucleotide sequence within the transgene. A barcode can be a diverged sequence, for example wherein the diverged nucleotide sequence within the transgene encodes the same amino acid sequence as a non-diverged (e.g., wild-type) nucleotide sequence but wherein the diverged nucleotide sequence comprises a different nucleotide sequence from the non-diverged (e.g., wild-type) nucleotide sequence, for example silent mutations. A diverged sequence can include, for example, one or more types of mutations, such as substitutions, when combined, generate a unique nucleotide sequence that translates into the wild-type amino acid sequence. A diverged sequence can be a recombinant sequence. A diverged sequence can be an engineered sequence. In some embodiments, the diverged nucleotide sequence is located at the junction of one or more transgene domains.

A barcode can encode the same amino acid sequence as a non-diverged nucleotide sequence, e.g., a reference wild-type amino acid sequence. A barcode can have at least about any of 80%, 85%, 90%, 91%, 92%, 93%, 94%, 95%, 96%, 97%, 98%, or 99% sequence identity to a wild-type nucleotide sequence. In some embodiments, a barcode is a probe binding site. A barcode can be of any suitable length, for example, in some embodiments, the barcode is an oligonucleotide between about 6 to about 30 nucleotides, such as about any of 6, 7, 8, 9, 10, 11, 12, 13, 14, 15, 20, 25, 26, 27, 28, 29, or 30 nucleotides in length. In some embodiments, the barcode can be between about 6 to about 10 nucleotides in length, between about 10 to about 20 nucleotides in length, between about 15 to about 25 nucleotides in length, or between about 20 to about 30 nucleotides in length. The barcode in the present disclosure is distinct from a sequence present in nature, for example present in a subject who may or may not comprise mutations from the reference wild-type sequence (i.e., alternative alleles), such that the barcode can accurately be used to detect the presence of a transgene. The barcode may be a naturally occurring amino acid sequence provided the nucleotide sequence present in the subject is different from the nucleotide sequence of the barcode that is comprising a naturally occurring amino acid sequence. In some embodiments, the first barcode and the second barcode are about the same nucleotide length.

In some embodiments, the barcode is randomly generated. For example, in some embodiments, the randomly generated barcode comprises the nucleotide sequence of NNNNNNNN wherein N refers to any of the nucleic acid bases A, T, C, and G, such as a nucleotide sequence set forth in any one of SEQ ID NOs: 12-17, or a nucleotide sequence comprising 1, 2, 3, 4, or 5 nucleotide substitutions, insertions, or deletions from the nucleotide sequence set forth in any one of SEQ ID NOs: 12-17. In some embodiments, the barcode comprises the nucleotide sequence set forth in any one of SEQ ID NOs: 12-17. In some embodiments, the barcode comprises a nucleotide sequence set forth in any one of SEQ ID NOs: 12-17, or comprises a nucleotide sequence comprising 1, 2, 3, 4, or 5 nucleotide substitutions, insertions, or deletions from the nucleotide sequence set forth in any one of SEQ ID NOs: 12-17, and the presence of the barcode indicates the presence of a transgene encoded by the vector comprising the barcode. In some embodiments, the barcode comprising the nucleotide sequence set forth in any one of SEQ ID NOs: 12-17 indicates the presence of a transgene encoded by the vector comprising the barcode.

a. Primer Binding Sites

In some embodiments, the first barcode and/or the second barcode is located within a first and/or second identifying region, wherein the first and/or second identifying region comprises primer binding sites that flank the first barcode and/or the second barcode. Primers are shorts, single-stranded pieces of DNA that are complementary to target sequences. Primer binding sites are a region of nucleotide sequence where an RNA or DNA single-stranded primer binds to start (i.e., initiate) replication. A forward primer binding site is a stretch of the antisense strand of DNA that runs in 3′ to 5′ direction and is complementary to a forward primer. A reverse primer binding site is a stretch of the sense strand of DNA that runs in the 5′ to 3′ direction and is complementary to a reverse primer. Primer binding sites generally include both a forward primer binding site and a reverse primer binding site, wherein the region between and inclusive of the primer binding sites are replicated, for example for amplification. In some instances, a forward primer binding site or a reverse primer binding site may be utilized alone, for example for sequencing.

A primer binding site can be of any suitable length, for example, in some embodiments, the primer binding site is a stretch of nucleotides between about 10 to about 30 nucleotides. In some embodiments, the primer binding site comprises about any of 10, 11, 12, 13, 14, 15, 16, 17, 18, 19, 20, 21, 22, 23, 24, 25, 26, 27, 28, 29, or 30 nucleotides. In some embodiments, the primer binding site comprises about 10 to about 15, about 12 to about 17, about 15 to about 20, about 17 to about 22, about 20 to about 25, about 22 to about 27, or about 25 to about 30 nucleotides.

Primer binding sites may comprise natural or non-natural sequences. Primer binding sites allow for the amplification a specific segment of DNA by, for example, polymerase chain reaction or for determining the nucleotide sequence of DNA by, for example, sequencing techniques such as Sanger sequencing. The primer binding sites of the present disclosure provide one way by which a barcode can be identified and/or detected to screen for the presence or absence of a transgene.

Primer binding sites may be complementary to universal primers. A universal primer is able to bind to a sequence found in many commonly used plasmid cloning vectors, many of which are derived from pUC vectors. Examples of universal primers include, but are not limited to M13 Reverse (−27), M13 Forward (−41), M13 Forward (−20), M13 Forward (−21), M13 Reverse (−48), SP6, T3, T7, T7 EEV, T7 Reverse, T7 Term, pBluescript KS, pBluescript SK, 3′pGEX, 5′pGEX, GST-Tag, pTrcHis-Forward, pTrcHis-Reverse, CMV-Forward, CMV-Reverse, EGFP-C, EGFP-N, BGH-Reverse, pQEproseq, pQErevseq, Intein Forward, 5′-pBabe-Seq, 3′-pBabe-Seq, -96 glll Sequencing Primer, GAL1 Forward, pBAD Forward, pBAD Reverse, pTRE 3′, pTRE 5′, pYESTrp Forward, pYESTrp Reverse, RVprimer3, Rvprimer4, GLprimer 1, GLprimer 2, SeqL-A (ATTL1), SeqL-B (ATTL2), SV40-pArev, SV40-Promoter, U6 Primer, Xpress Forward, EBV-Rev primer, hU6-01, hU6-02, 16S rRNA For, 16S rRNA Rev, 3′ RACE PCR, Anchored Oligo dT (20), Anchored Oligo dT (22), BGH Reverse, cDNA Cloning Primer, GAPDH For, GAPDH Rev, Neomycin For, Neomycin Rev, Oligo dT 15mer, Oligo dT 16mer, Oligo dT 18mer, Oligo dT 20mer, Oligo dT 20mer w/5′ Phos, PCMV Forward, pGEX 3′, pGEX 5′, Random Hexamer, Random Hexamer w/Biotin, and SP6 Upstream. In some embodiments, the universal primers are used to amplify the barcode sequence. In some embodiments, the universal primers are used to detect the barcode. In some embodiments, the detection of the barcode indicates the presence of a transgene encoded by a vector, wherein the vector comprises an identifying region comprising a barcode.

In some embodiments, the forward primer binding sites of the first barcode and the second barcode comprise the same sequence. In some embodiments, the forward primer binding site of the first barcode and/or the second barcode comprise different sequences. In some embodiments, the first and second forward primer binding sites are complementary to universal primers. In some embodiments, the first forward primer binding site and the second forward primer binding site is each between about 10 to about 30 nucleotides in length.

In some embodiments, the reverse primer binding sites of the first barcode and the second barcode comprise the same sequence. In some embodiments, the reverse primer binding site of the first barcode and/or the second barcode comprise different sequences. In some embodiments, the first and second reverse primer binding sites are complementary to universal primers. In some embodiments, the first reverse primer binding site and the second reverse primer binding site is each between about 10 to about 30 nucleotides in length.

In some embodiments, the forward primer binding sites of the first barcode and the second barcode comprise the same sequence, and the reverse primer binding sites of the first barcode and the second barcode comprise the same sequence. In some embodiments, the forward primer binding site of the first barcode and/or the second barcode comprise different sequences, and the reverse primer binding site of the first barcode and/or the second barcode comprise different sequences. In some embodiments, the first and second forward primer binding sites and the first and second reverse primer binding sites are complementary to universal primers. In some embodiments, the first forward primer binding site and the second forward primer binding site and the first reverse primer binding site and the second reverse primer binding site is each between about 10 to about 30 nucleotides in length.

In some embodiments, the forward and the reverse primer binding sites have a certain percentage complementarity with the primers that bind to each primer binding site. For example, in some embodiments, the forward primer binding site and the reverse primer binding site each comprises at least about 70% complementarity to the primer that binds to each primer binding site. In some embodiments, the forward binding site and the reverse primer binding site each comprises at least about 70%, about 80%, about 85%, about 90%, about 92%, about 95%, about 96%, about 97%, about 98%, about 99%, about 99.5%, or about 100% complementarity to the primer that binds to each primer binding site. In some embodiments, the forward binding site and the reverse primer binding site each comprises at least about 1, 2, 3, 4, 5, 6, 7, 8, 9, or 10 nucleotide substitutions, insertions, or deletions with respect to the nucleotide sequence of the primer that binds to each primer binding site. In some embodiments, the forward binding site and the reverse primer binding site each comprises 100% complementarity to the primer that binds to each primer binding site. In some embodiments, the primers bind to the primer binding site for amplification of the barcode.

In some embodiments, the distance between the forward and reverse primer binding sites is at least about 50 nucleotides in length, at least about 60 nucleotides in length, at least about 70 nucleotides in length, at least about 80 nucleotides in length, at least about 90 nucleotides in length, at least about 100 nucleotides in length, or more. In some embodiments, the distance between the forward and reverse primer binding sites is about 50 nucleotides to about 350 nucleotides in length, such as about 50 nucleotides to about 150 nucleotides, about 100 nucleotides to about 200 nucleotides, about 150 nucleotides to about 250 nucleotides, about 200 nucleotides to about 300 nucleotides, or about 250 nucleotides to about 350 nucleotides.

In some embodiments, the forward primer binding site and the reverse primer binding site are unique compared to nucleotide sequences found within the host genome. In some embodiments, the forward primer binding site and the reverse primer binding site are distinguishable from nucleotide sequences found within the host genome. In some embodiments, the primers that bind to the forward primer binding site and the reverse primer binding site do not substantially bind to nucleotide sequences found within the host genome. In some embodiments, the forward primer binding site comprises the nucleotide sequence of SEQ ID NO:33 or a variant thereof comprising about 1, 2, 3, 4, or 5 nucleotide substitutions, insertions, or deletions. In some embodiments, the reverse primer binding site comprises the nucleotide sequence of SEQ ID NO:34 or a variant thereof comprising about 1, 2, 3, 4, or 5 nucleotide substitutions, insertions, or deletions. In some embodiments, the forward primer binding site comprises the nucleotide sequence of SEQ ID NO:33, and/or the reverse primer binding site comprises the nucleotide sequence of SEQ ID NO:34.

b. Identifying Regions

In some embodiments, the nucleic acid(s), for example any of the vectors described herein or nucleic acids derived thereof that integrate into a cell genome, comprise an identifying region comprising a barcode. In some embodiments, the identifying region further comprises one or more primer binding sites, for example a forward primer binding site and/or a reverse primer binding site. In some embodiments, the barcode is located in between the forward primer binding site and the reverse primer binding site, i.e., the barcode is flanked by the forward primer binding site and the reverse primer binding site.

A vector may have one or more identifying regions. For example, a vector may comprise any of 1, 2, 3, 4, 5, 6, 7, 8, 9, 10, 11, 12, 13, 14, 15, 16, 17, 18, 19, 20, 21, 22, 23, 24, 25, or more identifying regions. The one or more identifying regions may comprise the same or different sequences. For example, a vector comprising one or more identifying regions wherein the identifying regions comprise the same sequences may be indicative of the presence of two identical copies inserted into the vector. In another example, a vector comprising one or more identifying regions wherein the identifying regions comprise difference sequences may be indicative of the presence of two different transgenes inserted into the vector. The identifying regions of the present disclosure are used to detect the presence of one or more barcodes that indicate the presence of one or more transgenes within an engineered cell or population of cells (e.g., T cells, B cells, NK cells, islet cells such as beta cells, or hypoimmunogenic cells thereof). For example, the identifying region may comprise any of 1, 2, 3, 4, 5, 6, 7, 8, 9, 10, 11, 12, 13, 14, 15, 16, 17, 18, 19, 20, 21, 22, 23, 24, 25, or more barcodes.

In some embodiments, the identifying region comprises one or more probe binding sites. In some embodiments, the probe is designed to bind to the barcode. Probe binding sites are stretches of nucleic acid that, for example, a fluorescein-labeled oligonucleotide probe can recognize and hybridize, as described in more detail in Section 11.1 above. Detection of the probe is also detection of the barcode, which indicate the presence of a transgene in a vector, including within a cell that contains or expresses the vector.

In some embodiments, the first identifying region and the second identifying region each has at least one barcode. In some embodiments, the first identifying region and/or the second identifying region has one barcode. In some embodiments, the first identifying region and/or the second identifying region has more than one barcode. In some embodiments, the first identifying region and/or the second identifying region comprises a first barcode and/or a second barcode comprising a nucleotide sequence set forth in any one of SEQ ID NOs:12-17, or a variant thereof having at least about 80% (such as at least about any of 80%, 85%, 90%, 91%, 92%, 93%, 94%, 95%, 96%, 97%, 98%, or 99%) sequence identity to any one of SEQ ID NOs:12-17. In some embodiments, the first identifying region and/or the second identifying region comprises a first barcode and/or a second barcode comprising a nucleotide sequence of any one of SEQ ID NOs:12-17, or a variant thereof comprising up to about 6 (such as about any of 1, 2, 3, 4, 5, or 6) nucleotide substitutions.

In some embodiments, the first identifying region and the second identifying region each comprises a probe binding site. In some embodiments, the first identifying region and/or the second identifying region comprises two or more probe binding sites. In some embodiments, the first identifying region and/or the second identifying region comprises a first probe binding site and/or a second probe binding site comprising a nucleotide sequence set forth in any one of SEQ ID NOs:18-23, or a variant thereof having at least about 80% (such as at least about any of 80%, 85%, 90%, 91%, 92%, 93%, 94%, 95%, 96%, 97%, 98%, or 99%) sequence identity to any one of SEQ ID NOs:18-23. In some embodiments, the first identifying region and/or the second identifying region comprises a first probe binding site and/or a second probe binding site comprising a nucleotide sequence of any one of SEQ ID NOs:18-23, or a variant thereof comprising up to about 6 (such as about any of 1, 2, 3, 4, 5, or 6) nucleotide substitutions.

In some embodiments, the first identifying region and the second identifying region are each between about 10 to about 100 nucleotides in length, such as about any of 10, 11, 12, 13, 14, 15, 16, 17, 18, 19, 20, 30, 40, 50, 60, 70, 80, 90, 91, 92, 93, 94, 95, 96, 97, 98, 99, or 100 nucleotides in length. In some embodiments, the first identifying region and the second identifying region are each between about 18 to about 30 nucleotides in length. In some embodiments, the first identifying region and the second identifying region are each at least about any of 18, 19, 20, 21, 22, 23, 24, 25, 26, 27, 28, 29, or 30 nucleotides in length. In some embodiments, the first identifying region and the second identifying region are about the same length. In some embodiments, the first identifying region and the second identifying region are different lengths. In some embodiments, the first identifying region and/or the second identifying region comprises a nucleotide sequence set forth in any one of SEQ ID NOs:24-30, or a variant thereof having at least about 80% (such as at least about any of 80%, 85%, 90%, 91%, 92%, 93%, 94%, 95%, 96%, 97%, 98%, or 99%) sequence identity to any one of SEQ ID NOs:24-30. In some embodiments, the first identifying region and/or the second identifying region comprises a nucleotide sequence of any one of SEQ ID NOs:24-30, or a variant thereof comprising up to about 6 (such as about any of 1, 2, 3, 4, 5, or 6) nucleotide substitutions. In some embodiments, the first identifying region and/or the second identifying region comprises an antisense nucleic acid sequence of SEQ ID NO:32, wherein NNNNNNNN is the barcode and wherein N represents a nucleotide selected from the group consisting of A, T, C, and G.

In some embodiments, the first identifying region is located in a non-coding region or a coding region of the vector comprising the first transgene. In some embodiments, the second identifying region is located in a second non-coding region or a second coding region of the vector comprising the second transgene. In some embodiments, the first identifying region and the second identifying region are located on the same vector. In some embodiments, the first identifying region and the second identifying region are located in the same region of the vector that comprises the first transgene and the second transgene. In some embodiments, the first identifying region and the second identifying region are located on different vectors.

In some embodiments, the first identifying region is located within the first transgene and/or the second identifying region is located within the second transgene. In some embodiments, the first identifying region is located outside of the first transgene and the second identifying region is located outside of the second transgene. In some embodiments, the first identifying region is located within the first transgene and the second identifying region is located outside of the second transgene. In some embodiments, the first identifying region is located within the first transgene and the second identifying region is located outside of the second transgene. In some embodiments, the first identifying region and the second identifying region are located on the same vector. In some embodiments, the first identifying region and the second identifying region are located in the same region of the vector that comprises the first transgene and the second transgene. In some embodiments, the first identifying region and the second identifying region are located on different vectors.

In some embodiments, the first identifying region is located 3′ to the first transgene and/or the second identifying region is located 3′ to the second transgene. In some embodiments, the first identifying region is located 3′ to the first transgene within about 1 to about 200 base pairs and/or the second identifying region is located 3′ to the second transgene within about 1 to about 200 base pairs. In some embodiments, the first identifying region is located 3′ to the first transgene at least about any of 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 191, 192, 193, 194, or 195 base pairs and/or the second identifying region is located 3′ to the second transgene at least about any of 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 191, 192, 193, 194, or 195 base pairs. In some embodiments, the first identifying region is located 3′ to the first transgene within 1-200 base pairs and/or the second identifying region is located 3′ to the transgene promoter within 1-200 base pairs.

In some embodiments, the first identifying region and/or the second identifying region is upstream of one or more additional regulatory elements. In some embodiments, the first identifying region and/or second identifying region is downstream of one or more additional regulatory elements. Regulatory elements can include, but are not limited to, any one or combination of: promoter sequences, enhancer sequences, intron sequences, terminator sequences, translation initiation signal sequences, polyadenylation signal sequences, replication element sequences, RNA processing and export element sequences, transposon sequences, transposase sequences, insulator sequences, 5′ UTR sequences, 3′ UTR sequences, mRNA 3′ end processing sequences, boundary element sequences, locus control region (LCR) sequences, matrix attachment region (MAR) sequences, ubiquitous chromatin opening elements, linker sequences, secretion signal sequences, anchoring peptide sequences, localization signal sequences, fusion tag sequences, affinity tag sequences, chaperonin sequences, protease sequences, and posttranscriptional regulatory element sequences.

In some embodiments, the first identifying region is upstream of a first promoter and/or the second identifying region is upstream of a second promoter. In some embodiments, the first identifying region is downstream of the first promoter and/or the second identifying region is downstream of the second promoter. In some embodiments, the first identifying region is upstream of the first promoter and the second identifying region is downstream of the second promoter. In some embodiments, the first identifying region is downstream of the first promoter and the second identifying region is upstream of the second promoter. In some embodiments, the promoter is selected from the group consisting of a CAG promoter, cytomegalovirus (CMV) promoter, EF1α promoter, EF1α short promoter, PGK promoter, adenovirus late promoter, vaccinia virus 7.5K promoter, SV40 promoter, tk promoter of HSV, mouse mammary tumor virus (MMTV) promoter, LTR promoter of HIV, Epstein Barr virus (EBV) promoter, Rous sarcoma virus (RSV) promoter, UBC promoter, MoMuLV promoter, an avian leukemia virus promoter, actin promoter, myosin promoter, hemoglobin promoter, creatine kinase promoter, hybrid CMV enhancer/chicken R-actin (CBA) promoter, and CBA hybrid intron (CBh) promoter. In some embodiments, the first promoter and/or the second promoter is operably linked to the first transgene and/or the second transgene.

In some embodiments, the first promoter is selected from the group consisting of: an EF1α promoter, an EF1α short promoter, a CAG promoter, a ubiquitin/S27a promoter, an SV40 early promoter, an adenovirus major late promoter, a mouse metallothionein-I promoter, an RSV promoter, an MMTV promoter, a Moloney murine leukemia virus Long Terminal repeat region, a CMV promoter, an actin promoter, an immunoglobulin promoter, a heat shock promoter, polyoma virus promoter, a fowlpox virus promoter, a bovine papilloma virus promoter, an avian sarcoma virus promoter, a retrovirus promoter, a hepatitis-B virus promoter, a PGK promoter, an adenovirus late promoter, a vaccinia virus 7.5K promoter, a SV40 promoter, a tk promoter of HSV, a mouse mammary tumor virus (MMTV) promoter, an LTR promoter of HIV, a promoter of moloney virus, an Epstein Barr virus (EBV) promoter, a Rous sarcoma virus (RSV) promoter, a U6 promoter, and an UBC promoter. In some embodiments, the second promoter is selected from the group consisting of: an EF1α promoter, an EF1α short promoter, a CAG promoter, a ubiquitin/S27a promoter, an SV40 early promoter, an adenovirus major late promoter, a mouse metallothionein-I promoter, an RSV promoter, an MMTV promoter, a Moloney murine leukemia virus Long Terminal repeat region, a CMV promoter, an actin promoter, an immunoglobulin promoter, a heat shock promoter, polyoma virus promoter, a fowlpox virus promoter, a bovine papilloma virus promoter, an avian sarcoma virus promoter, a retrovirus promoter, a hepatitis-B virus promoter, a PGK promoter, an adenovirus late promoter, a vaccinia virus 7.5K promoter, a SV40 promoter, a tk promoter of HSV, a mouse mammary tumor virus (MMTV) promoter, an LTR promoter of HIV, a promoter of moloney virus, an Epstein Barr virus (EBV) promoter, a Rous sarcoma virus (RSV) promoter, a U6 promoter, and an UBC promoter. In some embodiments, the first transgene is operably linked to a first promoter. In some embodiments, the second transgene is operably linked to a second promoter. In some embodiments, the first promoter and the second promoter comprise the same sequence.

In some embodiments, the first identifying region is located 5′ to the first promoter and/or the second identifying region is located 5′ to the second promoter. In some embodiments, the first identifying region is located 5′ to the first promoter within about 1 to about 200 base pairs and/or the second identifying region is located 5′ to the second promoter within about 1 to about 200 base pairs. In some embodiments, the first identifying region is located 5′ to the first promoter at least about any of 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 191, 192, 193, 194, or 195 base pairs and/or the second identifying region is located 5′ to the second promoter at least about any of 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 191, 192, 193, 194, or 195 base pairs. In some embodiments, the first identifying region is located 5′ to the first promoter within 1-200 base pairs and/or the second identifying region is located 5′ to the second promoter within 1-200 base pairs.

In some embodiments, the first identifying region is located 3′ of the first promoter and/or the second identifying region is located 3′ to the second promoter. In some embodiments, the first identifying region is located 3′ to the first promoter within about 1 to about 200 base pairs and/or the second identifying region is located 3′ to the second promoter within about 1 to about 200 base pairs. In some embodiments, the first identifying region is located 3′ to the first promoter at least about any of 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 191, 192, 193, 194, or 195 base pairs and/or the second identifying region is located 3′ to the second promoter at least about any of 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 191, 192, 193, 194, or 195 base pairs. In some embodiments, the first identifying region is located 3′ to the first promoter within 1-200 base pairs and/or the second identifying region is located 3′ to the second promoter within 1-200 base pairs.

In some embodiments, the first identifying region is located 5′ to the first promoter within about 1 to about 200 base pairs and/or the second identifying region is located 3′ to the second promoter within about 1 to about 200 base pairs. In some embodiments, the first identifying region is located 3′ to the first promoter within about 1 to about 200 base pairs and/or the second identifying region is located 5′ to the second promoter within about 1 to about 200 base pairs. In some embodiments, the first identifying region of the first vector is inserted 5′ to the first promoter, 3′ to the first transgene, and 5′ or 3′ to a Woodchuck Hepatitis Virus Posttranscriptional Regulatory Element (WPRE).

In some embodiments, a vector comprising a transgene and an identifying region comprising a barcode of the present disclosure comprises a linker sequence, such as any of an internal ribosome entry site (IRES) sequence, a cleavable peptide sequence, a 2A peptide sequence, a F2A peptide sequence, a E2A peptide sequence, a P2A peptide sequence, a T2A peptide sequence, or a tPT2A peptide sequence, or any other suitable linker sequence known in the art.

iv. Engineered Receptors

In some embodiments, the one or more transgenes comprise sequence(s) encoding an engineered receptor, such as a chimeric antigen receptor (CAR), a chimeric autoantibody receptor (CAAR), a B-cell autoantibody receptor (BAR), or a T cell receptor (TCR).

a. Chimeric Antigen Receptors (CARs)

In some embodiments, the one or more transgenes, e.g., the first and/or the second transgene, comprises a chimeric antigen receptor (CAR). CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. For example, a CAR may comprise an extracellular binding domain that specifically binds a target antigen, a transmembrane domain, and an intracellular signaling domain. In certain embodiments, the CAR may further comprise one or more additional elements, including one or more signal peptides, one or more extracellular hinge domains, and/or one or more intracellular costimulatory domains. Domains may be directly adjacent to one another, or there may be one or more amino acids linking the domains. The nucleotide sequence encoding a CAR may be derived from a mammalian sequence, for example, a mouse sequence, a primate sequence, a human sequence, or combinations thereof. In the cases where the nucleotide sequence encoding a CAR is non-human, the sequence of the CAR may be humanized. The nucleotide sequence encoding a CAR may also be codon-optimized for expression in a mammalian cell, for example, a human cell.

In some embodiments, the CAR is or comprises a first-generation CAR comprising an antigen binding domain, a transmembrane domain, and signaling domain (e.g., one, two or three signaling domains). In some embodiments, the CAR is or comprises a second-generation CAR comprising an antigen binding domain, a transmembrane domain, and two signaling domains. In some embodiments, the CAR is or comprises a third generation CAR comprising an antigen binding domain, a transmembrane domain, and at least three signaling domains. In some embodiments, the CAR is or comprises a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an scFv or Fab.

In some embodiments, the antigen binding domain binds to a cell surface antigen of a cell. In some embodiments, the antigen binding domain targets an antigen characteristic of a cell type, such as a neoplastic cell, a T cell, a B cell, or a senescent cell. In some embodiments, the antigen binding domain binds to a cell surface antigen of a cell. In some embodiments, a cell surface antigen is characteristic of one type of cell. In some embodiments, a cell surface antigen is characteristic of more than one type of cell.

In some embodiments, the antigen characteristic of a neoplastic cell is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, Epidermal Growth Factor Receptors (EGFR) (including ErbB1/EGFR, ErbB2/HER2, ErbB3/HER3, and ErbB4/HER4), Fibroblast Growth Factor Receptors (FGFR) (including FGF1, FGF2, FGF3, FGF4, FGF5, FGF6, FGF7, FGF18, and FGF21) Vascular Endothelial Growth Factor Receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2. EphB3, EphB4, and EphB6), CXCR1, CXCR2, CXCR3, CXCR4, CXCR6, CCR1, CCR2, CCR3, CCR4, CCR5, CCR6, CCR8, CFTR, CIC-1, CIC-2, CIC-4, CIC-5, CIC-7, CIC-Ka, CIC-Kb, Bestrophins, TMEM16A, GABA receptor, glycin receptor, ABC transporters, NAV1.1, NAV1.2, NAV1.3, NAV1.4, NAV1.5, NAV1.6, NAV1.7, NAV1.8, NAV1.9, sphingosin-1-phosphate receptor (S1P1R), NMDA channel, transmembrane protein, multispan transmembrane protein, T-cell receptor motifs, T-cell receptor alpha chains, T-cell receptor β chains, T-cell receptor γ chains, T-cell receptor δ chains, CCR7, CD3, CD4, CD5, CD7, CD8, CD11b, CD11c, CD16, CD19, CD20, CD21, CD22, CD25, CD28, CD34, CD35, CD40, CD45RA, CD45RO, CD52, CD56, CD62L, CD68, CD80, CD95, CD117, CD127, CD133, CD137 (4-1 BB), CD163, F4/80, IL-4Ra, Sca-1, CTLA-4, GITR, GARP, LAP, granzyme B, LFA-1, transferrin receptor, NKp46, perforin, CD4+, (e.g., CD4+Th1, Th2, Th17, Th40, Th22, Th9, Tfh, Canonical Treg, FoxP3+, Tr1, Th3, Treg17, or TREG cells) CDCP1, NT5E, EpCAM, CEA, gpA33, Mucins, TAG-72, Carbonic anhydrase IX, PSMA, Folate binding protein, Gangliosides (e.g., CD2, CD3, GM2), Lewis-γ2, VEGF, VEGFR 1/2/3, αVβ3, α5β1, ErbB1/EGFR, ErbB1/HER2, ErB3, c-MET, IGF1R, EphA3, TRAIL-R1, TRAIL-R2, RANKL, FAP, Tenascin, PDL-1, BAFF, HDAC, ABL, FLT3, KIT, MET, RET, IL-1β, ALK, RANKL, mTOR, CTLA-4, IL-6, IL-6R, JAK3, BRAF, PTCH, Smoothened, PIGF, ANPEP, TIMP1, PLAUR, PTPRJ, LTBR, or ANTXR1, Folate receptor alpha (FRa), ERBB2 (Her2/neu), EphA2, IL-13Ra2, epidermal growth factor receptor (EGFR), Mesothelin, TSHR, CD19, CD123, CD22, CD30, CD171, CS-1, CLL-1, CD33, EGFRvIII, GD2, GD3, BCMA, MUC16 (CA125), L1CAM, LeY, MSLN, IL13Rα1, L1-CAM, Tn Ag, prostate specific membrane antigen (PSMA), ROR1, FLT3, FAP, TAG72, CD38, CD44v6, CEA, EPCAM, B7H3, KIT, interleukin-11 receptor a (IL-IIRa), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-1 receptor, CAIX, LMP2, gplOO, bcr-abl, tyrosinase, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLACl, GloboH, NY-BR-1, UPK2, HAVCR1, ADRB3, PANX3, GPR20, LY6K, OR51E2, TARP, WT1, NY-ESO-1, LAGE-la, MAGE-A1, legumain, HPV E6, E7, ETV6-AML, sperm protein 17, XAGE1, Tie 2, MAD-CT-1, MAD-CT-2, Major histocompatibility complex class I-related gene protein (MR1), urokinase-type plasminogen activator receptor (uPAR), Fos-related antigen 1, p53, p53 mutant, prostein, survivin, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYPIB I, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, a neoantigen, CD133, CD15, CD184, CD24, CD56, CD26, CD29, CD44, HLA-A, HLA-B, HLA-C, (HLA-A,B,C) CD49f, CD151 CD340, CD200, tkrA, trkB, or trkC, or an antigenic fragment or antigenic portion thereof.

In some embodiments, the antigen characteristic of a T cell is selected from a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, AKT1, AKT2, AKT3, ATF2, BCL10, CALM1, CD3D (CD3δ), CD3E (CD3ε), CD3G (CD3γ), CD4, CD8, CD28, CD45, CD80 (B7-1), CD86 (B7-2), CD247 (CD3ζ), CTLA4 (CD152), ELK1, ERK1 (MAPK3), ERK2, FOS, FYN, GRAP2 (GADS), GRB2, HLA-DRA, HLA-DRB1, HLA-DRB3, HLA-DRB4, HLA-DRB5, HRAS, IKBKA (CHUK), IKBKB, IKBKE, IKBKG (NEMO), IL2, ITPR1, ITK, JUN, KRAS2, LAT, LCK, MAP2K1 (MEK1), MAP2K2 (MEK2), MAP2K3 (MKK3), MAP2K4 (MKK4), MAP2K6 (MKK6), MAP2K7 (MKK7), MAP3K1 (MEKK1), MAP3K3, MAP3K4, MAP3K5, MAP3K8, MAP3K14 (NIK), MAPK8 (JNK1), MAPK9 (JNK2), MAPK10 (JNK3), MAPK11 (p38β), MAPK12 (p38γ), MAPK13 (p38δ), MAPK14 (p38α), NCK, NFAT1, NFAT2, NFKB1, NFKB2, NFKBIA, NRAS, PAK1, PAK2, PAK3, PAK4, PIK3C2B, PIK3C3 (VPS34), PIK3CA, PIK3CB, PIK3CD, PIK3R1, PKCA, PKCB, PKCM, PKCQ, PLCY1, PRF1 (Perforin), PTEN, RAC1, RAF1, RELA, SDF1, SHP2, SLP76, SOS, SRC, TBK1, TCRA, TEC, TRAF6, VAV1, VAV2, or ZAP70.

In some embodiments, the antigen characteristic of senescent cells is, for example, urokinase-type plasminogen activator receptor (uPAR). In some embodiments, the antigen binding domain binds an antigen associated with a senescent cell. In some instances, the antigen is expressed by a senescent cell. In some embodiments, the CAR may be used for treatment or prophylaxis of disorders characterized by the aberrant accumulation of senescent cells, e.g., liver and lung fibrosis, atherosclerosis, diabetes and osteoarthritis.

In some embodiments, the antigen characteristic of a B cell is selected from the group consisting of IL-10, TGFβ, IgD, CD1, CD5, CD21, CD24, TLR4, CD21, CD22, CD23, Notch2, CD27, CXCR3, CXCR4, CXCR5, CXCR6, IgA, IgG, IgE, CD20, CD40, CD80, PDL-2, CD138, IL-6, CD38, CD78, CD319, CD25, CD30, CD19, CD22, ROR1, CD45, CD47, CD33, Igkappa, Iglambda, CD79a, CD79b, and IgM. In some embodiments, a CAR antigen binding domain binds to a ligand expressed on B cells, plasma cells, or plasmablasts, such as CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5, or CD2. See US 2003/0077249; WO 2017/058753; WO 2017/058850, the contents of which are herein incorporated by reference.

In some embodiments, the antigen binding domain targets an antigen characteristic of a disease, disorder, injury, or condition.

In certain embodiments, the antigen binding domain targets an antigen that is exclusively or preferentially expressed on tumor cells. Exemplary target antigens include, but are not limited to, CD5, CD19, CD20, CD22, CD23, CD30, CD70, Kappa, Lambda, B cell maturation agent (BCMA), G-protein coupled receptor family C group 5 member D (GPRC5D) (associated with leukemias); CS1/SLAMF7, CD38, CD138, GPRC5D, TACI, and BCMA (associated with myelomas); GD2, HER2, EGFR, EGFRvIII, B7H3, PSMA, PSCA, CAIX, CD171, CEA, CSPG4, EPHA2, FAP, FRa, IL-13Rα, Mesothelin, MUC1, MUC16, and ROR1 (associated with solid tumors).

In some embodiments, the antigen binding domain targets an antigen characteristic of an autoimmune or inflammatory disorder. In some embodiments, the CAR binds an antigen associated with an autoimmune or inflammatory disorder. In some instances, the antigen is expressed by a cell associated with an autoimmune or inflammatory disorder. In some embodiments, the autoimmune or inflammatory disorder is selected from the group consisting of: chronic graft-vs-host disease (GVHD), lupus, arthritis, immune complex glomerulonephritis, goodpasture, uveitis, hepatitis, systemic sclerosis or scleroderma, type I diabetes, multiple sclerosis, cold agglutinin disease, Pemphigus vulgaris, Grave's disease, autoimmune hemolytic anemia, Hemophilia A, Primary Sjogren's Syndrome, thrombotic thrombocytopenia purpura, neuromyelits optica, Evan's syndrome, IgM mediated neuropathy, cyroglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15(4):931-41) or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, or gene therapy. In some embodiments, the antigen characteristic of an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, the antigen binding domain targets an antigen characteristic of an infectious disease. In some embodiments, the CAR binds an antigen associated with an infectious disease. In some instances, the antigen is expressed by a cell affected by an infectious disease. In some embodiments, the infectious disease is selected from HIV, hepatitis B virus, hepatitis C virus, Human herpes virus, Human herpes virus 8 (HHV-8, Kaposi sarcoma-associated herpes virus (KSHV)), Human T-lymphotrophic virus-1 (HTLV-1), Merkel cell polyomavirus (MCV), Simian virus 40 (SV40), Epstein-Barr virus, CMV, or human papillomavirus. In some embodiments, the antigen characteristic of an infectious disease is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, HIV Env, gp120, or CD4-induced epitope on HIV-1 Env.

In some embodiments, the CAR comprises an antigen binding domain specific for any of: CD5, CD19, CD20, CD22, CD23, CD30, CD33, CD38, CD70, CD123, CD138, BCMA, GPRC5D, CD123, LeY, NKG2D ligand, WT1, GD2, HER2, EGFR, EGFRvIII, B7H3, PSMA, PSCA, CAIX, CD171, CEA, CSPG4, EPHA2, FAP, FRa, IL-13Rα, Mesothelin, MUC1, MUC16, ROR1, C-Met, CD133, Ep-CAM, GPC3, HPV16-E6, IL13Ra2, MAGEA3, MAGEA4, MART1, NY-ESO-1, VEGFR2, α-Folate receptor, CD24, CD44v7/8, EGP-2, EGP-40, erb-B2, erb-B 2,3,4, FBP, Fetal acethylcholine e receptor, GD2, GD3, HMW-MAA, IL-11Rα, KDR, Lewis Y, L1-cell adhesion molecule, MAGE-A1, Oncofetal antigen (h5T4), TAG-72, or CD19 and CD22. In some embodiments, the CAR comprises an antigen binding domain specific for any of: CD19, CD22, CD20, BCMA, an EBV antigen, CD27, CD30, CD19 and CD20, CD19 and CD22, CD19 and CD27, EBNA1, EBNA3A, BRLF1, BALF4, EBNA3C, LMP1, LMP2, LMP2A, LMP2B, BZLF1, BMLF1, gp350, gH/gL, EBNA1 and LMP1, EBNA1 and LMP2A, EBNA1 and LMP1 and LMP2A, LMP and BARF1 and EBNA1, CD19 and an EBV antigen, CD20 and an EBV antigen, or CD22 and an EBV antigen. In some embodiments, the CAR comprises an antigen binding domain specific for any of: CD19, CD20, CD22, CD38, CD123, CD138, BCMA, CD19 and CD22, CD19 and CD20, CD19 and BCMA, CD19 and BAFFR, CD33 and CD123, HER2 and B7H3, HER2 and EGFR, HER2 and IL13Rα, HER2 and ROR1, B7H3 and EGFR, B7H3 and IL13Rα, B7H3 and ROR1, EGFR and IL13Rα, and/or EGFR and ROR1.

In some embodiments, a cell comprises a CAR comprising an antigen binding domain specific for two or more target antigens. In some embodiments, a cell comprises a CAR comprising an antigen binding domain specific to two or more epitopes of the same target antigen. In some embodiments, a cell comprises two CARs each comprising a different antigen binding domain from each other. For example, in some embodiments, the two or more CARs each comprise an antigen binding domain specific for a target antigen such that the cell comprises two or more CARs targeting any combinations of CD19×CD20, CD19×BCMA, CD20×BCMA, CD19×CD22, CD19×BAFFR, CD33×CD123, HER2×B7H3, HER2×EGFR, HER2×IL13Rα, HER2×ROR1, B7H3×EGFR, B7H3×IL13Rα, B7H3×ROR1, EGFR×IL13Rα, EGFR×ROR1, and Her2×B7H3×EGFR×IL13Rα2×ROR1.

In some embodiments, the CAR comprises a transmembrane domain comprising at least a transmembrane region of the alpha, beta, or zeta chain of a T cell receptor, CD28, CD3 epsilon, CD45, CD4, CD5, CD8, CD9, CD16, CD22, CD33, CD3γ, CD64, CD80, CD86, CD134, CD137, CD154, or functional variant thereof. In some embodiments, the transmembrane domain comprises at least a transmembrane region(s) of CD8a, CD80, 4-1BB/CD137, CD28, CD34, CD4, FcFRIγ, CD16, OX40/CD134, CD3ζ, CD3ε, CD3γ, CD3δ, TCRα, TCRβ, TCRζ, CD32, CD64, CD64, CD45, CD5, CD9, CD22, CD3γ, CD80, CD86, CD40, CD40L/CD154, VEGFR2, FAS, FGFR2B, or functional variant thereof.

In some embodiments, the CAR comprises at least one signaling domain selected from one or more of B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, a CD3 zeta domain, an immunoreceptor tyrosine-based activation motif (ITAM), CD27, 4-1BB, CD134/OX40, CD30, CD40, PD-1, ICOS, LIGHT, NKG2C, a ligand that specifically binds with CD83, or functional fragment thereof.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the CAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine-serine doublets.

In some embodiments, the CAR comprises a transmembrane domain and a signaling domain. In some embodiments the signaling domain mediates downstream signaling during T cell activation.

In some embodiments, the CAR is a second-generation CAR. In some embodiments a second-generation CAR comprises an antigen binding domain, a transmembrane domain, and two signaling domains. In some embodiments a signaling domain mediates downstream signaling during T cell activation. In some embodiments a signaling domain is a costimulatory domain. In some embodiments, a costimulatory domain enhances cytokine production, CAR T cell proliferation, and or CAR T cell persistence during T cell activation.

In some some embodiments, the CAR is a third generation CAR. In some embodiments, a third generation CAR comprises an antigen binding domain, a transmembrane domain, and at least three signaling domains. In some embodiments a signaling domain mediates downstream signaling during T cell activation. In some embodiments a signaling domain is a costimulatory domain. In some embodiments, a costimulatory domain enhances cytokine production, CAR T cell proliferation, and or CAR T cell persistence during T cell activation. In some embodiments, a third generation CAR comprises at least two costimulatory domains. In some embodiments, the at least two costimulatory domains are not the same.

In some embodiments, the CAR is a fourth generation CAR. In some embodiments a fourth generation CAR comprises an antigen binding domain, a transmembrane domain, and at least two, three, or four signaling domains. In some embodiments a signaling domain mediates downstream signaling during T cell activation. In some embodiments a signaling domain is a costimulatory domain. In some embodiments, a costimulatory domain enhances cytokine production, CAR T cell proliferation, and or CAR T cell persistence during T cell activation.

In some embodiments, a first, second, third, or fourth generation CAR further comprises a domain which upon successful signaling of the CAR induces expression of a cytokine gene. In some embodiments, a cytokine gene is endogenous or exogenous to a target cell comprising a CAR which comprises a domain which upon successful signaling of the CAR induces expression of a cytokine gene. In some embodiments a cytokine gene encodes a pro-inflammatory cytokine. In some embodiments a cytokine gene encodes IL-1, IL-2, IL-9, IL-12, IL-18, TNF, IFN-gamma, or a functional fragment thereof. In some embodiments, a domain which upon successful signaling of the CAR induces expression of a cytokine gene is or comprises a transcription factor or functional domain or fragment thereof. In some embodiments, a domain which upon successful signaling of the CAR induces expression of a cytokine gene is or comprises a transcription factor or functional domain or fragment thereof. In some embodiments a transcription factor or functional domain or fragment thereof is or comprises a nuclear factor of activated T cells (NFAT), an NF-κB, or functional domain or fragment thereof. See, e.g., Zhang. C. et al., Engineering CAR-T cells. Biomarker Research. 5:22 (2017); WO 2016126608; Sha, H. et al., Chimaeric antigen receptor T-cell therapy for tumour immunotherapy. Bioscience Reports Jan. 27, 2017, 37 (1).

In some embodiments, a CAR antigen binding domain is or comprises an antibody or antigen-binding portion thereof. In some embodiments, a CAR antigen binding domain is or comprises an scFv or Fab. In some embodiments, a CAR antigen binding domain comprises an scFv or Fab fragment of a T-cell receptor alpha chain antibody, T-cell receptor β chain antibody, T-cell receptor γ chain antibody, T-cell receptor δ chain antibody, CCR7 antibody, CD3 antibody, CD4 antibody, CD5 antibody, CD7 antibody, CD8 antibody, CD11b antibody, CD11c antibody, CD16 antibody, CD19 antibody, CD20 antibody, CD21 antibody, CD22 antibody, CD25 antibody, CD28 antibody, CD34 antibody, CD35 antibody, CD40 antibody, CD45RA antibody, CD45RO antibody, CD52 antibody, CD56 antibody, CD62L antibody, CD68 antibody, CD80 antibody, CD95 antibody, CD117 antibody, CD127 antibody, CD133 antibody, CD137 (4-1 BB) antibody, CD163 antibody, F4/80 antibody, IL-4Ra antibody, Sea-1 antibody, CTLA-4 antibody, GITR antibody, GARP antibody, LAP antibody, granzyme B antibody, LFA-1 antibody, MR1 antibody, uPAR antibody, transferrin receptor antibody, or any combination thereof.

In some embodiments, a CAR antigen binding domain is or comprises an antibody or antigen-binding portion thereof. In some embodiments, a CAR antigen binding domain is or comprises an scFv or Fab. In some embodiments, the CAR encoded by the first transgene and/or the second transgene is selected from the group consisting of: a CD5-specific CAR, a CD19-specific CAR, a CD20-specific CAR, a CD22-specific CAR, a CD23-specific CAR, a CD30-specific CAR, a CD33-specific CAR, CD38-specific CAR, a CD70-specific CAR, a CD123-specific CAR, a CD138-specific CAR, a Kappa, Lambda, B cell maturation agent (BCMA)-specific CAR, a G-protein coupled receptor family C group 5 member D (GPRC5D)-specific CAR, a CD123-specific CAR, a LeY-specific CAR, a NKG2D ligand-specific CAR, a WT1-specific CAR, a GD2-specific CAR, a HER2-specific CAR, a EGFR-specific CAR, a EGFRvIII-specific CAR, a B7H3-specific CAR, a PSMA-specific CAR, a PSCA-specific CAR, a CAIX-specific CAR, a CD171-specific CAR, a CEA-specific CAR, a CSPG4-specific CAR, a EPHA2-specific CAR, a FAP-specific CAR, a FRa-specific CAR, a IL-13Rα-specific CAR, a Mesothelin-specific CAR, a MUC1-specific CAR, a MUC16-specific CAR, a ROR1-specific CAR, a C-Met-specific CAR, a CD133-specific CAR, a Ep-CAM-specific CAR, a GPC3-specific CAR, a HPV16-E6-specific CAR, a IL13Rα2-specific CAR, a MAGEA3-specific CAR, a MAGEA4-specific CAR, a MART1-specific CAR, a NY-ESO-1-specific CAR, a VEGFR2-specific CAR, a α-Folate receptor-specific CAR, a CD24-specific CAR, a CD44v7/8-specific CAR, a EGP-2-specific CAR, a EGP-40-specific CAR, a erb-B2-specific CAR, a erb-B 2,3,4-specific CAR, a FBP-specific CAR, a Fetal acethylcholine e receptor-specific CAR, a GD2-specific CAR, a GD3-specific CAR, a HMW-MAA-specific CAR, a IL-11Rα-specific CAR, a KDR-specific CAR, a Lewis Y-specific CAR, a L1-cell adhesion molecule-specific CAR, a MAGE-A1-specific CAR, a Oncofetal antigen (h5T4)-specific CAR, a TAG-72-specific CAR, and a CD19/CD22-bispecific CAR.

In some embodiments, a CAR comprises a signaling domain which is a costimulatory domain. In some embodiments, a CAR comprises a second costimulatory domain. In some embodiments, a CAR comprises at least two costimulatory domains. In some embodiments, a CAR comprises at least three costimulatory domains. In some embodiments, a CAR comprises a costimulatory domain selected from the group consisting of CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof. In some embodiments, if a CAR comprises two or more costimulatory domains, two costimulatory domains are different. In some embodiments, if a CAR comprises two or more costimulatory domains, two costimulatory domains are the same.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the CAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3-zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments the intracellular signaling domain includes intracellular components of a 4-1BB signaling domain and a CD3-zeta signaling domain. In some embodiments, the intracellular signaling domain includes intracellular components of a CD28 signaling domain and a CD3-zeta signaling domain.

In some embodiments, the CAR comprises an extracellular antigen binding domain (e.g., antibody or antibody fragment, such as an scFv or Fab) that binds to an antigen (e.g., tumor antigen), a spacer (e.g., containing a hinge domain, such as any as described herein), a transmembrane domain (e.g., any as described herein), and an intracellular signaling domain (e.g., any intracellular signaling domain, such as a primary signaling domain or costimulatory signaling domain as described herein). In some embodiments, the intracellular signaling domain is or includes a primary cytoplasmic signaling domain. In some embodiments, the intracellular signaling domain additionally includes an intracellular signaling domain of a costimulatory molecule (e.g., a costimulatory domain).

In some embodiments, the CAR contains one or more domains that combine an antigen- or ligand-binding domain (e.g., antibody or antibody fragment) that provides specificity for a desired antigen (e.g., tumor antigen) with intracellular signaling domains. In some embodiments, the intracellular signaling domain is a stimulating or an activating intracellular domain portion, such as a T cell stimulating or activating domain, providing a primary activation signal or a primary signal. In some embodiments, the intracellular signaling domain contains or additionally contains a costimulatory signaling domain to facilitate effector functions. In some embodiments, chimeric receptors when genetically engineered into immune cells can modulate T cell activity, and, in some cases, can modulate T cell differentiation or homeostasis, thereby resulting in genetically engineered cells or populations of cells (e.g., T cells, B cells, NK cells, islet cells such as beta cells, or hypoimmunogenic cells thereof) with improved longevity, survival and/or persistence in vivo, such as for use in adoptive cell therapy methods.

Exemplary antigen receptors, including CARs, and methods for engineering and introducing such receptors into cells, include those described, for example, in W0200014257, WO2013126726, WO2012/129514, WO2014031687, WO2013/166321, WO2013/071154, WO2013/123061, U.S. patent app. Pub. Nos. US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent app. No. EP2537416, and/or those described by Sadelain et al., Cancer Discov. 2013 April; 3(4): 388-398; Davila et al., (2013) PLoS ONE 8(4): e61338; Turtle et al., Curr. Opin. Immunol., 2012 October; 24(5): 633-39; Wu et al., Cancer, 2012 March 18(2): 160-75. In some aspects, the antigen receptors include a CAR as described in U.S. Pat. No. 7,446,190, and those described in WO/2014055668. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., (2013) Nature Reviews Clinical Oncology, 10, 267-276; Wang et al., (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282. The recombinant receptors, such as CARs, generally include an extracellular antigen binding domain, such as a portion of an antibody molecule, generally a variable heavy (VH) chain region and/or variable light (VL) chain region of the antibody, e.g., an scFv antibody fragment. In some embodiments, the antigen binding domain of the CAR molecule comprises an antibody, an antibody fragment, an scFv, a Fv, a Fab, a (Fab′)2, a single domain antibody (SdAb), a VH or VL domain, or a camelid VHH domain.

In addition to the CARs described herein, various chimeric antigen receptors and nucleotide sequences encoding the same are known in the art and would be suitable for use according to the present disclosure. See, e.g., WO2013040557; WO2012079000; WO2016030414; Smith T, et al., Nature Nanotechnology. 2017. DOI: 10.1038/NNANO.2017.57, the disclosures of which are herein incorporated by reference.

In some embodiments, the antigen targeted by the CAR is a polypeptide. In some embodiments, it is a carbohydrate or other molecule. In some embodiments, the antigen is selectively expressed or overexpressed on cells of a disease or condition, e.g., the tumor or pathogenic cells, as compared to normal or non-targeted cells or tissues. In other embodiments, the antigen is expressed on normal cells and/or is expressed on the engineered cells or populations of cells (e.g., T cells, B cells, NK cells, islet cells such as beta cells, or hypoimmunogenic cells thereof).

In some embodiments, the antigen targeted by the receptor includes antigens associated with a B cell malignancy, such as any of a number of known B cell markers. In some embodiments, the antigen targeted by the receptor is CD20, CD19, CD22, ROR1, CD45, CD47, CD21, CD5, CD33, Igkappa, Iglambda, CD79a, CD79b, or CD30.

In some embodiments, a cell comprises a CAR comprising an antigen binding domain specific for two or more target antigens. In some embodiments, a cell comprises a CAR comprising an antigen binding domain specific to two or more epitopes of the same target antigen. In some embodiments, a cell comprises two or more CARs each comprising a different antigen binding domain from each other. In some embodiments, the CAR binds to CD19. In some embodiments, the CAR binds to CD22. In some embodiments, the CAR binds to CD19 and CD22. In some embodiments, the CAR binds to CD20. In some embodiments, the CAR binds to CD19 and CD20. In some embodiments, the CAR binds to BCMA. In some embodiments, the CAR binds to CD19 and BCMA. In some embodiments, the CAR binds to BAFFR. In some embodiments, the CAR binds to CD19 and BAFFR. In some embodiments, the CAR binds to CD33. In some embodiments, the CAR binds to CD123. In some embodiments, the CAR binds to CD33 and CD123. In some embodiments, the CAR binds to HER2. In some embodiments, the CAR binds to B7H3. In some embodiments, the CAR binds to HER2 and B7H3. In some embodiments, the CAR binds to EGFR. In some embodiments, the CAR binds to HER2 and EGFR. In some embodiments, the CAR binds to IL13Rα1 and/or IL13Rα2 (i.e., IL13Rα). In some embodiments, the CAR binds to HER2 and IL13Rα. In some embodiments, the CAR binds to ROR1. In some embodiments, the CAR binds to HER2 and ROR1. In some embodiments, the CAR binds to B7H3 and EGFR. In some embodiments, the CAR binds to B7H3 and IL13Rα. In some embodiments, the CAR binds to B7H3 and ROR1. In some embodiments, the CAR binds to EGFR and IL13Rα. In some embodiments, the CAR binds to EGFR and ROR1. For example, in some embodiments, the two or more CARs each comprise an antigen binding domain specific for a target antigen such that the cell comprises two or more CARs targeting any combinations of CD19×CD20, CD19×BCMA, CD20×BCMA, CD19×CD22, CD19×BAFFR, CD33×CD123, HER2×B7H3, HER2×EGFR, HER2×IL13Rα, HER2×ROR1, B7H3×EGFR, B7H3×IL13Rα, B7H3×ROR1, EGFR×IL13Rα, EGFR×ROR1, and Her2×B7H3×EGFR×IL13Rα2×ROR1. In some embodiments, the CAR(s) targets an autoimmune disease. In some embodiments, the CAR(s) targets leukemia or lymphoma. In some embodiments, the CAR(s) targets acute myeloid leukemia. In some embodiments, the CAR(s) targets a solid tumor malignancy. In some embodiments, the CAR is selected from the group consisting of a first-generation CAR, a second generation CAR, a third generation CAR, and a fourth generation CAR. In some embodiments, the CAR includes a single binding domain that binds to a single target antigen. In some embodiments, the CAR includes a single binding domain that binds to more than one target antigen, e.g., 2, 3, or more target antigens. In some embodiments, the CAR includes two binding domains such that each binding domain binds to a different target antigen. In some embodiments, the CAR includes two binding domains such that each binding domain binds to the same target antigen. For example, detailed descriptions of exemplary CARs including CD19-specific, CD22-specific and CD19/CD22-bispecific CARs can be found in WO2012/079000, WO2016/149578, and WO2020/014482, the disclosures including the sequence listings and figures are incorporated herein by reference in their entirety.

In some embodiments, the antigen targeted by the antigen-binding domain is CD19. In some aspects, the antigen-binding domain of the recombinant receptor, e.g., CAR, and the antigen-binding domain binds, such as specifically binds or specifically recognizes, a CD19, such as a human CD19. In some embodiments, the scFv contains a VH and a VL derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723. In some embodiments, the scFv is derived from FMC63. FMC63 generally refers to a mouse monoclonal IgG1 antibody raised against Naim-1 and -16 cells expressing CD19 of human origin (Fing, N. R., et al., (1987). Leucocyte typing III. 302).

In some embodiments, the chimeric antigen receptor includes an extracellular portion containing an antibody or antibody fragment, e.g., as described above. In some embodiments, the chimeric antigen receptor includes an extracellular portion containing the antibody or fragment and an intracellular signaling domain.

In some embodiments of any of the CARs described herein, the antibody or fragment includes an scFv or a single-domain antibody fragment, for example, a VHH. In certain embodiments, the scFv may comprise a heavy chain variable region (VH) and a light chain variable region (VL) of an antibody connected by a linker. The VH and the VL may be connected in either order, i.e., VH-linker-VL or VL-linker-VH. Non-limiting examples of linkers include Whitlow linker, (G4S)n (n can be a positive integer, e.g., 1, 2, 3, 4, 5, 6, etc.) linker, and variants thereof.

In some embodiments, the antibody portion of a recombinant receptor, e.g., CAR, further includes spacer between the transmembrane domain and extracellular antigen binding domain. In some embodiments, the spacer includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers include, but are not limited to, those described in Hudecek et al., (2013) Clin. Cancer Res., 19:3153, WO2014031687, U.S. Pat. No. 8,822,647 or published app. No. US 2014/0271635. In some embodiments, the constant region or portion is of a human IgG, such as IgG4 or IgG1.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an ITAM. For example, in some aspects, the antigen recognition domain (e.g., extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g., scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e., comprise at least the transmembrane region(s) of) the alpha, beta or zeta chain of the T-cell receptor, CD28, CD3 epsilon, CD45, CD4, CD5, CD8, CD9, CD16, CD22, CD33, CD3γ, CD64, CD80, CD86, CD 134, CD 137, CD 154. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28.

In some embodiments, the extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

The receptor, e.g., the CAR, generally includes at least one intracellular signaling component or components. In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAMs containing primary cytoplasmic signaling sequences include those derived from CD3-zeta chain, FcR gamma, CD3 gamma, CD3 delta, and CD3 epsilon. In some embodiments, cytoplasmic signaling molecule(s) in the CAR contain(s) a cytoplasmic signaling domain, portion thereof, or sequence derived from CD3-zeta.

In some embodiments, the receptor includes an intracellular component of a TCR complex, such as a TCR CD3 chain that mediates T-cell activation and cytotoxicity, e.g., CD3-zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the intracellular component is or includes a CD3-zeta intracellular signaling domain. In some embodiments, the intracellular component is or includes a signaling domain from Fc receptor gamma chain. In some embodiments, the receptor, e.g., CAR, includes the intracellular signaling domain and further includes a portion, such as a transmembrane domain and/or hinge portion, of one or more additional molecules such as CD8, CD4, CD25, or CD16. For example, in some aspects, the CAR or other chimeric receptor is a chimeric molecule of CD3-zeta (CD3-z) or Fc receptor and a portion of one of CD8, CD4, CD25, or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 4-1BB. In some aspects, the T cell costimulatory molecule is 4-1BB.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3-zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments the intracellular signaling domain includes intracellular components of a 4-1BB signaling domain and a CD3-zeta signaling domain. In some embodiments, the intracellular signaling domain includes intracellular components of a CD28 signaling domain and a CD3-zeta signaling domain.

In some embodiments, a CD19 specific CAR includes an anti-CD19 single-chain antibody fragment (scFv), a transmembrane domain such as one derived from human CD8a, a 4-1BB (CD137) co-stimulatory signaling domain, and a CD3ζ signaling domain. In some embodiments, a CD22 specific CAR includes an anti-CD22 scFv, a transmembrane domain such as one derived from human CD8a, a 4-1BB (CD137) co-stimulatory signaling domain, and a CD3ζ signaling domain. In some embodiments, a CD19/CD22-bispecific CAR includes an anti-CD19 scFv, an anti-CD22 scFv, a transmembrane domain such as one derived from human CD8a, a 4-1BB (CD137) co-stimulatory signaling domain, and a CD3ζ signaling domain.

In some embodiments, the CAR comprises a commercial CAR construct carried by a T cell. Non-limiting examples of commercial CAR-T cell based therapies include brexucabtagene autoleucel (TECARTUS®), axicabtagene ciloleucel (YESCARTA®), idecabtagene vicleucel (ABECMA®), lisocabtagene maraleucel (BREYANZI®), tisagenlecleucel (KYMRIAH®), Descartes-08 and Descartes-11 from Cartesian Therapeutics, CTL110 from Novartis, P-BMCA-101 from Poseida Therapeutics, AUTO4 from Autolus Limited, UCARTCS from Cellectis, PBCAR19B and PBCAR269A from Precision Biosciences, FT819 from Fate Therapeutics, and CYAD-211 from Clyad Oncology.

In some embodiments, the CAR comprises an extracellular antigen binding domain (e.g., antibody or antibody fragment, such as an scFv) that binds to an antigen (e.g., tumor antigen), a spacer (e.g., containing a hinge domain, such as any as described herein), a transmembrane domain (e.g., any as described herein), and an intracellular signaling domain (e.g., any intracellular signaling domain, such as a primary signaling domain or costimulatory signaling domain as described herein). In some embodiments, the intracellular signaling domain is or includes a primary cytoplasmic signaling domain. In some embodiments, the intracellular signaling domain additionally includes an intracellular signaling domain of a costimulatory molecule (e.g., a costimulatory domain).

### Exemplary CAR Components

Exemplary components of CARs that may be used in the present disclosure are described below.

In some embodiments, the sequences of each component in a CAR can include any combination listed in Table 1.

TABLE 1
CAR components and Exemplary Sequences.

| | | SEQ ID |
|---|---|---|
| Component | Sequence | NO: |
| Extracellular | | |
| binding domain | | |
| Anti-CD19 scFv | DIQMTQTTSSLSASLGDRVTISCRASQDISKYLNWYQ | 255 |
| (FMC63) | QKPDGTVKLLIYHTSRLHSGVPSRFSGSGSGTDYSLT | |
| | ISNLEQEDIATYFCQQGNTLPYTFGGGTKLEITGSTS | |
| | GSGKPGSGEGSTKGEVKLQESGPGLVAPSQSLSVTC | |
| | TVSGVSLPDYGVSWIRQPPRKGLEWLGVIWGSETTY | |
| | YNSALKSRLTIIKDNSKSQVFLKMNSLQTDDTAIYY | |
| | CAKHYYYGGSYAMDYWGQGTSVTVSS | |
| Anti-CD19 scFv | DIQMTQTTSSLSASLGDRVTISCRASQDISKYLNWYQ | 256 |
| (FMC63) | QKPDGTVKLLIYHTSRLHSGVPSRFSGSGSGTDYSLT | |
| | ISNLEQEDIATYFCQQGNTLPYTFGGGTKLEITGGGG | |
| | SGGGGSGGGGSEVKLQESGPGLVAPSQSLSVTCTVS | |
| | GVSLPDYGVSWIRQPPRKGLEWLGVIWGSETTYYNS | |
| | ALKSRLTIIKDNSKSQVFLKMNSLQTDDTAIYYCAK | |
| | HYYYGGSYAMDYWGQGTSVTVSS | |
| Spacer (e.g., | | |
| hinge) | | |
| IgG4 Hinge | ESKYGPPCPPCP | 257 |
| CD8 Hinge | TTTPAPRPPTPAPTIASQPLSLRPE | 258 |
| CD28 Hinge | IEVMYPPPYLDNEKSNGTIIHVKGKHLCPSPLFPGPS | 259 |
| | KP | |
| Transmembrane | | |
| CD8 | ACRPAAGGAVHTRGLDFACDIYIWAPLAGTCGVLL | 260 |
| transmembrane | LSLVITLYC | |
| domain | | |
| CD28 | FWVLVVVGGVLACYSLLVTVAFIIFWV | 261 |
| transmembrane | | |
| domain | | |
| Costimulatory | | |
| domain | | |
| CD28 | RSKRSRLLHSDYMNMTPRRPGPTRKHYQPYAPPRDF | 262 |
| costimulatory | AAYRS | |
| domain | | |
| 4-1BB | KRGRKKLLYIFKQPFMRPVQTTQEEDGCSCRFPEEE | 263 |
| costimulatory | EGGCEL | |
| domain | | |
| Primary Signaling | | |
| Domain | | |
| CD3-zeta | RVKFSRSADAPAYQQGQNQLYNELNLGRREEYDVL | 264 |
| | DKRRGRDPEMGGKPRRKNPQEGLYNELQKDKMAE | |
| | AYSEIGMKGERRRGKGHDGLYQGLSTATKDTYDAL | |
| | HMQALPPR | |
| CD3-zeta | RVKFSRSADAPAYKQGQNQLYNELNLGRREEYDVL | 265 |
| | DKRRGRDPEMGGKPRRKNPQEGLYNELQKDKMAE | |
| | AYSEIGMKGERRRGKGHDGLYQGLSTATKDTYDAL | |
| | HMQALPPR | |

Signal peptides: In certain embodiments, the CAR may comprise a signal peptide at the N-terminus. Non-limiting examples of signal peptides include CD8a signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 2 below.

TABLE 2
Exemplary sequences of signal peptides.

| SEQ ID | | |
|---|---|---|
| NO: | Sequence | Description |
| 266 | MALPVTALLLPLALLLHAARP | CD8α signal peptide |
| 267 | METDTLLLWVLLLWVPGSTG | IgK signal peptide |
| 268 | MLLLVTSLLLCELPHPAFLL | GMCSFR-α (CSF2RA) |
| | IP | signal peptide |

Hinge or pacer domains: In certain embodiments, the CAR may comprise a hinge domain, also referred to as a spacer. The terms “hinge” and “spacer” may be used interchangeably in the present disclosure. Non-limiting examples of hinge domains include CD8α hinge domain, CD28 hinge domain, IgG4 hinge domain, IgG4 hinge-CH2-CH3 domain, and variants thereof, the amino acid sequences of which are provided in Table 3 below.

TABLE 3
Exemplary sequences of hinge domains.

| SEQ ID | | |
|---|---|---|
| NO: | Sequence | Description |
| 269 | TTTPAPRPPTPAPTIAS | CD8α hinge domain |
| | QPLSLRPEACRPAAGGA | |
| | VHTRGLDFACD | |
| 259 | IEVMYPPPYLDNEKSNG | CD28 hinge domain |
| | TIIHVKGKHLCPSPLFP | |
| | GPSKP | |
| 270 | AAAIEVMYPPPYLDNEK | CD28 hinge domain |
| | SNGTIIHVKGKHLCPSP | |
| | LFPGPSKP | |
| 257 | ESKYGPPCPPCP | IgG4 hinge domain |
| 271 | ESKYGPPCPSCP | IgG4 hinge domain |
| 272 | ESKYGPPCPPCPAPEFL | IgG4 hinge-CH2-CH3 |
| | GGPSVFLFPPKPKDTLM | domain |
| | ISRTPEVTCVVVDVSQE | |
| | DPEVQFNWYVDGVEVHN | |
| | AKTKPREEQFNSTYRVV | |
| | SVLTVLHQDWLNGKEYK | |
| | CKVSNKGLPSSIEKTIS | |
| | KAKGQPREPQVYTLPPS | |
| | QEEMTKNQVSLTCLVKG | |
| | FYPSDIAVEWESNGQPE | |
| | NNYKTTPPVLDSDGSFF | |
| | LYSRLTVDKSRWQEGNV | |
| | FSCSVMHEALHNHYTQK | |
| | SLSLSLGK | |

Transmembrane domains: In certain embodiments, the transmembrane domain of the CAR may comprise a transmembrane region of the alpha, beta, or zeta chain of a T cell receptor, CD28, CD3ε, CD45, CD4, CD5, CD8, CD9, CD16, CD22, CD33, CD3γ, CD64, CD80, CD86, CD134, CD137, CD154, or a functional variant thereof, including the human versions of each of these sequences. In other embodiments, the transmembrane domain may comprise a transmembrane region of CD8ax, CD803, 4-1BB/CD137, CD28, CD34, CD4, FcεRIγ, CD16, OX40/CD134, CD3, CD3ε, CD3γ, CD3δ, TCRα˜, TCRβ3, TCR, CD32, CD64, CD64, CD45, CD5, CD9, CD22, CD3γ, CD80, CD86, CD40, CD40L/CD154, VEGFR2, FAS, FGFR2B, or a functional variant thereof, including the human versions of each of these sequences. In some embodiments, the transmembrane domain is selected from the group consisting of: alpha, beta, or zeta chain of a T cell receptor, CD28, CD3ε, CD45, CD4, CD5, CD8, CD9, CD16, CD22, CD33, CD3γ, CD64, CD80, CD86, CD134, CD137, CD154, CD8α, CD80, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD3γ, CD80, CD86, CD40, CD40L/CD154, VEGFR2, FAS, FGFR2B, and any functional variant thereof.

TABLE 4
Exemplary sequences of transmembrane domains.

| SEQ ID | | |
|---|---|---|
| NO: | Sequence | Description |
| 273 | IYIWAPLAGTCG | CD8α transmembrane domain |
| | VLLLSLVITLYC | |
| 261 | FWVLVVVGGVLA | CD28 transmembrane domain |
| | CYSLLVTVAFII | |
| | FWV | |
| 274 | MFWVLVVVGGVL | CD28 transmembrane domain |
| | ACYSLLVTVAFI | |
| | IFWV | |

Intracellular domains: In certain embodiments, the intracellular signaling domain and/or intracellular costimulatory domain of the CAR may comprise one or more signaling domains selected from the group consisting of: B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3ζ, an immunoreceptor tyrosine-based activation motif (ITAM), CD27, 4-1BB, CD134/OX40, CD30, CD40, PD-1, ICOS, LIGHT, NKG2C, a ligand that specifically binds with CD83, and a functional variant thereof, including the human versions of each of these sequences. In some embodiments, the intracellular signaling domain and/or intracellular costimulatory domain comprises one or more signaling domains selected from a CD3 domain, an ITAM, a CD28 domain, 4-1BB domain, or a functional variant thereof. Table 5 provides the amino acid sequences of a few exemplary intracellular costimulatory and/or signaling domains. In certain embodiments, the CD3 signaling domain may have a mutation, e.g., a glutamine (Q) to lysine (K) mutation, at amino acid position 14.

TABLE 5
Exemplary sequences of intracellular costimulatory
and/or signaling domains.

| SEQ ID | | |
|---|---|---|
| NO: | Sequence | Description |
| 254 | KRGRKKLLYIFKQPFMRPVQTTQEEDGC | 4-1BB costimulatory |
| | SCRFPEEEEGGCEL | domain |
| 253 | RSKRSRLLHSDYMNMTPRRPGPTRKHYQ | CD28 costimulatory |
| | PYAPPRDFAAYRS | domain |
| 264 | RVKFSRSADAPAYQQGQNQLYNELNLGR | CD3ζ signaling domain |
| | REEYDVLDKRRGRDPEMGGKPRRKNPQ | |
| | EGLYNELQKDKMAEAYSEIGMKGERRR | |
| | GKGHDGLYQGLSTATKDTYDALHMQAL | |
| | PPR | |
| 265 | RVKFSRSADAPAYKQGQNQLYNELNLGR | CD3ζ signaling |
| | REEYDVLDKRRGRDPEMGGKPRRKNPQ | domain (with |
| | EGLYNELQKDKMAEAYSEIGMKGERRR | Q to K mutation |
| | GKGHDGLYQGLSTATKDTYDALHMQAL | at position 14) |
| | PPR | |

Markers: In some embodiments, the antigen receptor further includes a marker and/or cells expressing the CAR or other antigen receptor further includes a surrogate marker, such as a cell surface marker, which may be used to confirm transduction or engineering of the cell to express the receptor. In some embodiments, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence. In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred. In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

### Exemplary CD19 CARs

In some embodiments, the CAR is a CD19 CAR (“CD19-CAR”). In some embodiments, the CD19 CAR may comprise an extracellular binding domain that specifically binds CD19 and one or more of: any of the signal peptides described herein, any of the hinge domains described herein, any of the transmembrane domains described herein, any of the intracellular costimulatory domains described herein, and/or any of the intracellular signaling domains described herein, e.g., in tandem.

In some embodiments, the signal peptide of the CD19 CAR comprises a CD8a signal peptide. In some embodiments, the signal peptide comprises an IgK signal peptide. In some embodiments, the signal peptide comprises a GMCSFR-α or CSF2RA signal peptide. In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv. In some embodiments, the extracellular binding domain of the CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34(16-17):1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 6 below. In some embodiments, the CD19-specific scFv may comprise one or more CDRs having the CDR amino acid sequences set forth in Table 6. In some embodiments, the CD19-specific scFv may comprise a light chain with one or more CDRs having the light chain CDR amino acid sequences set forth in Table 6. In some embodiments, the CD19-specific scFv may comprise a heavy chain with one or more CDRs having the heavy chain CDR amino acid sequences set forth in Table 6. In any of these embodiments, the CD19-specific scFv may comprise one or more CDRs comprising one or more amino acid substitutions or comprising a sequence that is at least about 80% identical (e.g., at least about 80%, at least about 85%, at least about 90%, at least about 95%, at least about 96%, at least about 97%, at least about 98%, at least about 99%, or about 100% identical), to any of the sequences identified in Table 6. In some embodiments, the extracellular binding domain of the CD19 CAR comprises or consists of the one or more CDRs as described herein. In some embodiments, the linker linking the V_{H} and the V_{L} portions of the scFv is a Whitlow linker having an amino acid sequence set forth in Table 6. In some embodiments, the Whitlow linker may be replaced by a different linker, for example, a 3×G₄S linker having an amino acid sequence set forth in Table 6.

TABLE 6
Exemplary sequences of anti-CD19 scFv and components.

| SEQ ID NO: | Amino Acid Sequence | Description |
|---|---|---|
| 255 | DIQMTQTTSSLSASLGDRVTISCRAS | Anti-CD19 FMC63 scFv |
| | QDISKYLNWYQQKPDGTVKLLIYHTS | entire sequence, with |
| | RLHSGVPSRFSGSGSGTDYSLTISN | Whitlow linker |
| | LEQEDIATYFCQQGNTLPYTFGGGT | |
| | KLEITGSTSGSGKPGSGEGSTKGEVK | |
| | LQESGPGLVAPSQSLSVTCTVSGVSL | |
| | PDYGVSWIRQPPRKGLEWLGVIWGS | |
| | ETTYYNSALKSRLTIIKDNSKSQVFL | |
| | KMNSLQTDDTAIYYCAKHYYYGGS | |
| | YAMDYWGQGTSVTVSS | |
| 275 | DIQMTQTTSSLSASLGDRVTISCRAS | Anti-CD19 FMC63 scFv |
| | QDISKYLNWYQQKPDGTVKLLIYHTS | light chain variable |
| | RLHSGVPSRFSGSGSGTDYSLTISNL | region |
| | EQEDIATYFCQQGNTLPYTFGGGT | |
| | KLEIT | |
| 276 | QDISKY | Anti-CD19 FMC63 scFv |
| | | light chain CDR1 |
| | HTS | Anti-CD19 FMC63 scFv |
| | | light chain CDR2 |
| 278 | QQGNTLPYT | Anti-CD19 FMC63 scFv |
| | | light chain CDR3 |
| 279 | GSTSGSGKPGSGEGSTKG | Whitlow linker |
| 280 | EVKLQESGPGLVAPSQSLSVTCTVS | Anti-CD19 FMC63 scFv |
| | GVSLPDYGVSWIRQPPRKGLEWLGV | heavy chain variable |
| | IWGSETTYYNSALKSRLTIIKDNSK | region |
| | SQVFLKMNSLQTDDTAIYYCAKHY | |
| | YYGGSYAMDYWGQGTSVTVSS | |
| 281 | GVSLPDYG | Anti-CD19 FMC63 scFv |
| | | heavy chain CDR1 |
| 282 | IWGSETT | Anti-CD19 FMC63 scFv |
| | | heavy chain CDR2 |
| 283 | AKHYYYGGSYAMDY | Anti-CD19 FMC63 scFv |
| | | heavy chain CDR3 |
| 256 | DIQMTQTTSSLSASLGDRVTISCRA | Anti-CD19 FMC63 scFv |
| | SQDISKYLNWYQQKPDGTVKLLIYH | entire sequence, with |
| | TSRLHSGVPSRFSGSGSGTDYSLTI | 3xG4S linker |
| | SNLEQEDIATYFCQQGNTLPYTFGG | |
| | GTKLEITGGGGSGGGGSGGGGSEVK | |
| | LQESGPGLVAPSQSLSVTCTVSGVS | |
| | LPDYGVSWIRQPPRKGLEWLGVIWG | |
| | SETTYYNSALKSRLTIIKDNSKSQV | |
| | FLKMNSLQTDDTAIYYCAKHYYYGG | |
| | SYAMDYWGQGTSVTVSS | |
| 284 | GGGGSGGGGSGGGGS | 3xG₄S linker |

In some embodiments, the extracellular binding domain of the CD 19 CAR is derived from an antibody specific to CD 19, including, for example, SJ25C1 (Bejcek et al., Cancer Res. 55:2346-2351 (1995)), HD37 (Pezutto et al., J. Immunol. 138(9):2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmnacol. Exp. Ther. 335:213-222 (2010)), BU12 (Callard et al., J. Immunology, 148(10): 2983-2987 (1992)), and CLB-CD19 (De Rie Cell. Immunol. 118:368-381(1989)). In any of these embodiments, the extracellular binding domain of the CD19 CAR can comprise or consist of the V_{H}, the V_{L}, and/or one or more CDRs of any of the antibodies.

In some embodiments, the hinge domain of the CD19 CAR comprises a CD8a hinge domain, for example, a human CD8a hinge domain. In some embodiments, the hinge domain comprises a CD28 hinge domain, for example, a human CD28 hinge domain. In some embodiments, the hinge domain comprises an IgG4 hinge domain, for example, a human IgG4 hinge domain. In some embodiments, the hinge domain comprises a IgG4 hinge-Ch2-Ch3 domain, for example, a human IgG4 hinge-Ch2-Ch3 domain. In some embodiments, the transmembrane domain of the CD19 CAR comprises a CD8a transmembrane domain, for example, a human CD8a transmembrane domain. In some embodiments, the transmembrane domain comprises a CD28 transmembrane domain, for example, a human CD28 transmembrane domain.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the intracellular costimulatory domain comprises a CD28 costimulatory domain. CD28 is another co-stimulatory molecule on T cells. In some embodiments, the CD28 costimulatory domain is human. In some embodiments, the intracellular costimulatory domain of the CD19 CAR comprises a 4-1BB costimulatory domain and a CD28 costimulatory domain as described. In some embodiments, the intracellular signaling domain of the CD19 CAR comprises a CD3-zeta (ζ) signaling domain. CD3-zeta associates with T cell receptors (TCRs) to produce a signal and contains immunoreceptor tyrosine-based activation motifs (ITAMs). The CD3-zeta signaling domain refers to amino acid residues from the cytoplasmic domain of the zeta chain that are sufficient to functionally transmit an initial signal necessary for T cell activation. In some embodiments, the CD3-zeta signaling domain is human.

In some embodiments, the CD19 CAR comprises a CD19-specific scFv, a CD8a hinge domain, a CD8a transmembrane domain, a 4-1BB costimulatory domain, a CD3ζ signaling domain, and/or variants (i.e., having a sequence that is at least about 80% identical, for example, at least about 80%, at least about 85%, at least about 90%, at least about 95%, at least about 96%, at least about 97%, at least about 98%, or at least about 99% identity) thereof. In any of these embodiments, the CD19 CAR may additionally comprise a signal peptide (e.g., a CD8a signal peptide) as described above.

In some embodiments, the CD19 CAR comprises a CD19-specific scFv, an IgG4 hinge domain, a CD28 transmembrane domain, a 4-1BB costimulatory domain, a CD3ζ signaling domain, and/or variants (i.e., having a sequence that is at least about 80% identical, for example, at least about 80%, at least about 85%, at least about 90%, at least about 95%, at least about 96%, at least about 97%, at least about 98%, or at least about 99% identity) thereof. In any of these embodiments, the CD19 CAR may additionally comprise a signal peptide (e.g., a CD8a signal peptide) as described above.

In some embodiments, the CD19 CAR comprises a CD19-specific scFv, a CD28 hinge domain, a CD28 transmembrane domain, a CD28 costimulatory domain, a CD3ζ signaling domain, and/or variants (i.e., having a sequence that is at least about 80% identical, for example, at least about 80%, at least about 85%, at least about 90%, at least about 95%, at least about 96%, at least about 97%, at least about 98%, or at least about 99% identity) thereof. In any of these embodiments, the CD19 CAR may additionally comprise a signal peptide (e.g., a CD8a signal peptide) as described above.

In some embodiments, the CD19 CAR is encoded by the sequence set forth in SEQ ID NO: 285 or a sequence at least about 80% identical (e.g., at least about 80%, at least about 85%, at least about 90%, at least about 95%, at least about 96%, at least about 97%, at least about 98%, at least about 99%, or about 100% identical) to the nucleotide sequence set forth in SEQ ID NO: 285 (see Table 7). The encoded CD19 CAR has a corresponding amino acid sequence set forth in SEQ ID NO: 286 or is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in of SEQ ID NO: 286, with the following components: CD8a signal peptide, FMC63 scFv (V_{L}—Whitlow linker-V_{H}), CD8a hinge domain, CD8a transmembrane domain, 4-1BB costimulatory domain, and CD3ζ signaling domain.

TABLE 7
Exemplary sequences of CD19 CARs.

| SEQ ID NO: | Sequence | Description |
|---|---|---|
| 285 | atggccttaccagtgaccgccttgctcctgccgctggcct | Exemplary CD19 |
| | tgctgctccacgccgccaggccggacatccagatgacaca | CAR nucleotide |
| | gactacatcctccctgtctgcctctctgggagacagagtc | sequence |
| | accatcagttgcagggcaagtcaggacattagtaaatatt | |
| | taaattggtatcagcagaaaccagatggaactgttaaact | |
| | cctgatctaccatacatcaagattacactcaggagtccca | |
| | tcaaggttcagtggcagtgggtctggaacagattattctc | |
| | tcaccattagcaacctggagcaagaagatattgccactta | |
| | cttttgccaacagggtaatacgcttccgtacacgttcgga | |
| | ggggggaccaagctggagatcacaggctccacctctggat | |
| | ccggcaagcccggatctggcgagggatccaccaagggcga | |
| | ggtgaaactgcaggagtcaggacctggcctggtggcgccc | |
| | tcacagagcctgtccgtcacatgcactgtctcaggggtct | |
| | cattacccgactatggtgtaagctggattcgccagcctcc | |
| | acgaaagggtctggagtggctgggagtaatatggggtagt | |
| | gaaaccacatactataattcagctctcaaatccagactga | |
| | ccatcatcaaggacaactccaagagccaagttttcttaaa | |
| | aatgaacagtctgcaaactgatgacacagccatttactac | |
| | tgtgccaaacattattactacggtggtagctatgctatgg | |
| | actactggggccaaggaacctcagtcaccgtctcctcaac | |
| | cacgacgccagcgccgcgaccaccaacaccggcgcccacc | |
| | atcgcgtcgcagcccctgtccctgcgcccagaggcgtgcc | |
| | ggccagcggcggggggcgcagtgcacacgagggggctgga | |
| | cttcgcctgtgatatctacatctgggcgcccttggccggg | |
| | acttgtggggtccttctcctgtcactggttatcacccttt | |
| | actgcaaacggggcagaaagaaactcctgtatatattcaa | |
| | acaaccatttatgagaccagtacaaactactcaagaggaa | |
| | gatggctgtagctgccgatttccagaagaagaagaaggag | |
| | gatgtgaactgagagtgaagttcagcaggagcgcagacgc | |
| | ccccgcgtaccagcagggccagaaccagctctataacgag | |
| | ctcaatctaggacgaagagaggagtacgatgttttggaca | |
| | agagacgtggccgggaccctgagatggggggaaagccgag | |
| | aaggaagaaccctcaggaaggcctgtacaatgaactgcag | |
| | aaagataagatggcggaggcctacagtgagattgggatga | |
| | aaggcgagcgccggaggggcaaggggcacgatggccttta | |
| | ccagggtctcagtacagccaccaaggacacctacgacgcc | |
| | cttcacatgcaggccctgccccctcgc | |
| 286 | MALPVTALLLPLALLLHAARPDIQMTQTTS | Exemplary CD19 |
| | SLSASLGDRVTISCRASQDISKYLNWYQQK | CAR amino acid |
| | PDGTVKLLIYHTSRLHSGVPSRFSGSGSGT | sequence |
| | DYSLTISNLEQEDIATYFCQQGNTLPYTFG | |
| | GGTKLEITGSTSGSGKPGSGEGSTKGEVKL | |
| | QESGPGLVAPSQSLSVTCTVSGVSLPDYGV | |
| | SWIRQPPRKGLEWLGVIWGSETTYYNSALK | |
| | SRLTIIKDNSKSQVFLKMNSLQTDDTAIYY | |
| | CAKHYYYGGSYAMDYWGQGTSVTVSSTTTP | |
| | APRPPTPAPTIASQPLSLRPEACRPAAGGA | |
| | VHTRGLDFACDIYIWAPLAGTCGVLLLSLV | |
| | ITLYCKRGRKKLLYIFKQPFMRPVQTTQEE | |
| | DGCSCRFPEEEEGGCELRVKFSRSADAPAY | |
| | QQGQNQLYNELNLGRREEYDVLDKRRGRDP | |
| | EMGGKPRRKNPQEGLYNELQKDKMAEAYSE | |
| | IGMKGERRRGKGHDGLYQGLSTATKDTYDA | |
| | LHMQALPPR | |

### Exemplary CD20 CARs

In some embodiments, the CAR is a CD20 CAR (“CD20-CAR”). CD20 is an antigen found on the surface of B cells as early at the pro-B phase and progressively at increasing levels until B cell maturity, as well as on the cells of most B-cell neoplasms. CD20 positive cells are also sometimes found in cases of Hodgkin's disease, myeloma, and thymoma. In some embodiments, the CD20 CAR may comprise an extracellular binding domain that specifically binds CD20 and one or more of: any of the signal peptides described herein, any of the hinge domains described herein, any of the transmembrane domains described herein, any of the intracellular costimulatory domains described herein, and/or any of the intracellular signaling domains described herein, e.g., in tandem.

In some embodiments, the signal peptide of the CD20 CAR comprises a CD8a signal peptide. In some embodiments, the signal peptide comprises an IgK signal peptide. In some embodiments, the signal peptide comprises a GMCSFR-α or CSF2RA signal peptide.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Leu16, IFS, 1.5.3, rituximab, obinutuzumab, ibritumomab, ofatumumab, tositumumab, odronextamab, veltuzumab, ublituximab, and ocrelizumab. In any of these embodiments, the extracellular binding domain of the CD20 CAR can comprise or consist of the V_{H}, the V_{L}, and/or one or more CDRs of any of the antibodies.

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14(12):1025-1033 (2001). In some embodiments, the linker is a 3×G₄S linker. In other embodiments, the linker is a Whitlow linker as described herein. In some embodiments, the amino acid sequences of different portions of the entire Len16-derived scFv (also referred to as Len16 scFv) and its different portions are provided in Table 8 below. In some embodiments, the CD20-specific scFv may comprise one or more CDRs having the CDR amino acid sequences set forth in Table 8. In some embodiments, the CD20-specific scFv may comprise a light chain with one or more CDRs having light chain CDR amino acid sequences set forth in Table 8. In some embodiments, the CD20-specific scFv may comprise a heavy chain with one or more CDRs having heavy chain CDR amino acid sequences set forth in Table 8. In any of these embodiments, the CD20-specific scFv may comprise one or more CDRs comprising one or more amino acid substitutions, or comprising a sequence that is at least about 80% identical (e.g., at least about 80%, at least about 85%, at least about 90%, at least about 95%, at least about 96%, at least about 97%, at least about 98%, at least about 99%, or about 100% identity), to any of the sequences identified. In some embodiments, the extracellular binding domain of the CD20 CAR comprises or consists of the one or more CDRs as described herein.

TABLE 8
Exemplary sequences of anti-CD20 scFv and components.

| SEQ ID NO: | Amino Acid Sequence | Description |
|---|---|---|
| 293 | DIVLTQSPAILSASPGEKVTMTCRAS | Anti-CD20 Leu16 scFv |
| | SSVNYMDWYQKKPGSSPKPWIYATS | entire sequence, with |
| | NLASGVPARFSGSGSGTSYSLTISR | Whitlow linker |
| | VEAEDAATYYCQQWSFNPPTFGGGT | |
| | KLEIKGSTSGSGKPGSGEGSTKGEV | |
| | QLQQSGAELVKPGASVKMSCKASGY | |
| | TFTSYNMHWVKQTPGQGLEWIGAIY | |
| | PGNGDTSYNQKFKGKATLTADKSSS | |
| | TAYMQLSSLTSEDSADYYCARSNYY | |
| | GSSYWFFDVWGAGTTVTVSS | |
| 294 | DIVLTQSPAILSASPGEKVTMTCRA | Anti-CD20 Leu16 scFv |
| | SSSVNYMDWYQKKPGSSPKPWIYAT | light chain variable |
| | SNLASGVPARFSGSGSGTSYSLTIS | region |
| | RVEAEDAATYYCQQWSFNPPTFGGG | |
| | TKLEIK | |
| 295 | RASSSVNYMD | Anti-CD20 Leu16 scFv |
| | | light chain CDR1 |
| 296 | ATSNLAS | Anti-CD20 Leu16 scFv |
| | | light chain CDR2 |
| 297 | QQWSFNPPT | Anti-CD20 Leu16 scFv |
| | | light chain CDR3 |
| 298 | EVQLQQSGAELVKPGASVKMSCKA | Anti-CD20 Leu16 scFv |
| | SGYTFTSYNMHWVKQTPGQGLEWI | heavy chain |
| | GAIYPGNGDTSYNQKFKGKATLTA | |
| | DKSSSTAYMQLSSLTSEDSADYYC | |
| | ARSNYYGSSYWFFDVWGAGTTVTV | |
| | SS | |
| 299 | SYNMH | Anti-CD20 Leu16 scFv |
| | | heavy chain CDR1 |
| 300 | AIYPGNGDTSYNQKFKG | Anti-CD20 Leu16 scFv |
| | | heavy chain CDR2 |
| 301 | SNYYGSSYWFFDV | Anti-CD20 Leu16 scFv |
| | | heavy chain CDR3 |

In some embodiments, the hinge domain of the CD20 CAR comprises a CD8α hinge domain, for example, a human CD8α hinge domain. In some embodiments, the hinge domain comprises a CD28 hinge domain, for example, a human CD28 hinge domain. In some embodiments, the hinge domain comprises an IgG4 hinge domain, for example, a human IgG4 hinge domain. In some embodiments, the hinge domain comprises a IgG4 hinge-Ch2-Ch3 domain, for example, a human IgG4 hinge-Ch2-Ch3 domain.

In some embodiments, the transmembrane domain of the CD20 CAR comprises a CD8α transmembrane domain, for example, a human CD8α transmembrane domain. In some embodiments, the transmembrane domain comprises a CD28 transmembrane domain, for example, a human CD28 transmembrane domain.

In some embodiments, the intracellular costimulatory domain of the CD20 CAR comprises a 4-1BB costimulatory domain, for example, a human 4-1BB costimulatory domain. In some embodiments, the intracellular costimulatory domain comprises a CD28 costimulatory domain, for example, a human CD28 costimulatory domain.

In some embodiments, the intracellular signaling domain of the CD20 CAR comprises a CD3 zeta (ζ) signaling domain, for example, a human CD3ζ signaling domain.

In some embodiments, the CAR is a CD20 CAR, including, for example, a CD20 CAR comprising a CD20-specific scFv, a CD8a hinge domain, a CD8a transmembrane domain, a 4-1BB costimulatory domain, a CD3ζ signaling domain, and/or variants (i.e., having a sequence that is at least about 80% identical, for example, at least about 80%, at least about 85%, at least about 90%, at least about 95%, at least about 96%, at least about 97%, at least about 98%, or at least about 99% identity) thereof. In some embodiments, the first barcode and/or the second barcode is located at the junction of the CD20-specific scFv and the CD8a hinge domain. In some embodiments, the first barcode and/or the second barcode is located at the junction of the CD8a hinge domain and the CD28 transmembrane domain. In some embodiments, the first barcode and/or the second barcode is located at the junction of the CD28 transmembrane domain and the 4-1BB costimulatory domain. In some embodiments, the first barcode and/or the second barcode is located at the junction of the 4-1BB costimulatory domain and the CD3ζ signaling domain.

In some embodiments, the CAR is a CD20 CAR, including, for example, a CD20 CAR comprising a CD20-specific scFv, a CD28 hinge domain, a CD8a transmembrane domain, a 4-1BB costimulatory domain, a CD3ζ signaling domain, and/or variants (i.e., having a sequence that is at least about 80% identical, for example, at least about 80%, at least about 85%, at least about 90%, at least about 95%, at least about 96%, at least about 97%, at least about 98%, or at least about 99% identity) thereof. In some embodiments, the first barcode and/or the second barcode is located at the junction of the CD20-specific scFv and the CD28 hinge domain. In some embodiments, the first barcode and/or the second barcode is located at the junction of the CD28 hinge domain and the CD8a transmembrane domain. In some embodiments, the first barcode and/or the second barcode is located at the junction of the CD8a transmembrane domain and the 4-1BB costimulatory domain. In some embodiments, the first barcode and/or the second barcode is located at the junction of the 4-1BB costimulatory domain and the CD3ζ signaling domain.

In some embodiments, the CAR is a CD20 CAR, including, for example, a CD20 CAR comprising a CD20-specific scFv, an IgG4 hinge domain, a CD8a transmembrane domain, a 4-1BB costimulatory domain, a CD3ζ signaling domain, and/or variants (i.e., having a sequence that is at least about 80% identical, for example, at least about 80%, at least about 85%, at least about 90%, at least about 95%, at least about 96%, at least about 97%, at least about 98%, or at least about 99% identity) thereof. In some embodiments, the first barcode and/or the second barcode is located at the junction of the CD20-specific scFv and the IgG4 hinge domain. In some embodiments, the first barcode and/or the second barcode is located at the junction of the IgG4 hinge domain and the CD8a transmembrane domain. In some embodiments, the first barcode and/or the second barcode is located at the junction of the CD8a transmembrane domain and the 4-1BB costimulatory domain. In some embodiments, the first barcode and/or the second barcode is located at the junction of the 4-1BB costimulatory domain and the CD3ζ signaling domain.

In some embodiments, the CAR is a CD20 CAR, including, for example, a CD20 CAR comprising a CD20-specific scFv2, a CD8a hinge domain, a CD28 transmembrane domain, a 4-1BB costimulatory domain, a CD3ζ signaling domain, and/or variants (i.e., having a sequence that is at least about 80% identical, for example, at least about 80%, at least about 85%, at least about 90%, at least about 95%, at least about 96%, at least about 97%, at least about 98%, or at least about 99% identity) thereof. In some embodiments, the first barcode and/or the second barcode is located at the junction of the CD20-specific scFv2 and the CD8a hinge domain. In some embodiments, the first barcode and/or the second barcode is located at the junction of the CD8a hinge domain and the CD28 transmembrane domain. In some embodiments, the first barcode and/or the second barcode is located at the junction of the CD28 transmembrane domain and the 4-1BB costimulatory domain. In some embodiments, the first barcode and/or the second barcode is located at the junction of the 4-1BB costimulatory domain and the CD3ζ signaling domain.

In some embodiments, the CAR is a CD20 CAR, including, for example, a CD20 CAR comprising a CD20-specific scFv, a CD28 hinge domain, a CD28 transmembrane domain, a 4-1BB costimulatory domain, a CD3ζ signaling domain, and/or variants (i.e., having a sequence that is at least about 80% identical, for example, at least about 80%, at least about 85%, at least about 90%, at least about 95%, at least about 96%, at least about 97%, at least about 98%, or at least about 99% identity) thereof. In some embodiments, the first barcode and/or the second barcode is located at the junction of the CD20-specific scFv and the CD28 hinge domain. In some embodiments, the first barcode and/or the second barcode is located at the junction of the CD28 hinge domain and the CD28 transmembrane domain. In some embodiments, the first barcode and/or the second barcode is located at the junction of the CD28 transmembrane domain and the 4-1BB costimulatory domain. In some embodiments, the first barcode and/or the second barcode is located at the junction of the 4-1BB costimulatory domain and the CD3ζ signaling domain.

In some embodiments, the CAR is a CD20 CAR, including, for example, a CD20 CAR comprising a CD20-specific scFv, an IgG4 hinge domain, a CD28 transmembrane domain, a 4-1BB costimulatory domain, a CD3ζ signaling domain, and/or variants (i.e., having a sequence that is at least about 80% identical, for example, at least about 80%, at least about 85%, at least about 90%, at least about 95%, at least about 96%, at least about 97%, at least about 98%, or at least about 99% identity) thereof. In some embodiments, the first barcode and/or the second barcode is located at the junction of the CD20-specific scFv and the IgG4 hinge domain. In some embodiments, the first barcode and/or the second barcode is located at the junction of the IgG4 hinge domain and the CD28 transmembrane domain. In some embodiments, the first barcode and/or the second barcode is located at the junction of the CD28 transmembrane domain and the 4-1BB costimulatory domain. In some embodiments, the first barcode and/or the second barcode is located at the junction of the 4-1BB costimulatory domain and the CD3ζ signaling domain.

### Exemplary CD22 CARs

In some embodiments, the CAR is a CD22 CAR (“CD22-CAR”). CD22 is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise an extracellular binding domain that specifically binds CD22 and one or more of: any of the signal peptides described herein, any of the hinge domains described herein, any of the transmembrane domains described herein, any of the intracellular costimulatory domains described herein, and/or any of the intracellular signaling domains described herein, e.g., in tandem.

In some embodiments, the signal peptide of the CD22 CAR comprises a CD8a signal peptide. In some embodiments, the signal peptide comprises an IgK signal peptide. In some embodiments, the signal peptide comprises a GMCSFR-α or CSF2RA signal peptide.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, or pinatuzumab. In any of these embodiments, the extracellular binding domain of the CD22 CAR can comprise or consist of the V_{H}, the V_{L}, and/or one or more CDRs of any of the antibodies.
In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a 3×G₄S linker. In other embodiments, the Whitlow linker may be used instead. In some embodiments, the amino acid sequences of the entire m971-derived scFv (also referred to as m971 scFv) and its different portions are provided in Table 9 below. In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 μM). In some embodiments, the scFv derived from m971-L7 comprises the V_{H} and the V_{L} of m971-L7 connected by a 3×G₄S linker. In other embodiments, the Whitlow linker may be used instead. In some embodiments, the amino acid sequences of the entire m971-L7-derived scFv (also referred to as m971-L7 scFv) and its different portions are provided in Table 9 below. In some embodiments, the CD22-specific scFv may comprise one or more CDRs having CDR amino acid sequences set forth in Table 9. In some embodiments, the CD22-specific scFv may comprise a heavy chain with one or more CDRs having heavy chain CDR amino acid sequences set forth in Table 9. In some embodiments, the CD22-specific scFv may comprise a light chain with one or more CDRs having light chain CDR amino acid sequences set forth in Table 9. In any of these embodiments, the CD22-specific scFv may comprise one or more CDRs comprising one or more amino acid substitutions, or comprising a sequence that is at least about 80% identical (e.g., at least about 80%, at least about 85%, at least about 90%, at least about 95%, at least about 96%, at least about 97%, at least about 98%, at least about 99%, or about 100% identical), to any of the sequences identified. In some embodiments, the extracellular binding domain of the CD22 CAR comprises or consists of the one or more CDRs as described herein.

TABLE 9
Exemplary sequences of anti-CD22 scFv and components.

| SEQ ID NO: | Amino Acid Sequence | Description |
|---|---|---|
| 302 | QVQLQQSGPGLVKPSQTLSLTCAISG | Anti-CD22 m971 scFv |
| | DSVSSNSAAWNWIRQSPSRGLEWL | entire sequence, with |
| | GRTYYRSKWYNDYAVSVKSRITINP | 3xG4S linker |
| | DTSKNQFSLQLNSVTPEDTAVYYCA | |
| | REVTGDLEDAFDIWGQGTMVTVSS | |
| | GGGGSGGGGSGGGGSDIQMTQSPSS | |
| | LSASVGDRVTITCRASQTIWSYLNW | |
| | YQQRPGKAPNLLIYAASSLQSGVPS | |
| | RFSGRGSGTDFTLTISSLQAEDFATY | |
| | YCQQSYSIPQTFGQGTKLEIK | |
| 303 | QVQLQQSGPGLVKPSQTLSLTCAISG | Anti-CD22 m971 scFv |
| | DSVSSNSAAWNWIRQSPSRGLEWL | heavy chain variable |
| | GRTYYRSKWYNDYAVSVKSRITINP | region |
| | DTSKNQFSLQLNSVTPEDTAVYYCA | |
| | REVTGDLEDAFDIWGQGTMVTVSS | |
| 304 | GDSVSSNSAA | Anti-CD22 m971 scFv |
| | | heavy chain CDR1 |
| 305 | TYYRSKWYN | Anti-CD22 m971 scFv |
| | | heavy chain CDR2 |
| 306 | AREVTGDLEDAFDI | Anti-CD22 m971 scFv |
| | | heavy chain CDR3 |
| 307 | DIQMTQSPSSLSASVGDRVTITCRAS | Anti-CD22 m971 scFv |
| | QTIWSYLNWYQQRPGKAPNLLIYAA | light chain |
| | SSLQSGVPSRFSGRGSGTDFTLTISS | |
| | LQAEDFATYYCQQSYSIPQTFGQGT | |
| | KLEIK | |
| 308 | QTIWSY | Anti-CD22 m971 scFv |
| | | light chain CDR1 |
| | AAS | Anti-CD22 m971 scFv |
| | | light chain CDR2 |
| 310 | QQSYSIPQT | Anti-CD22 m971 scFv |
| | | light chain CDR3 |
| 311 | QVQLQQSGPGMVKPSQTLSLTCAIS | Anti-CD22 m971-L7 |
| | GDSVSSNSVAWNWIRQSPSRGLEWL | scFv entire sequence, |
| | GRTYYRSTWYNDYAVSMKSRITINP | with 3xG4S linker |
| | DTNKNQFSLQLNSVTPEDTAVYYCA | |
| | REVTGDLEDAFDIWGQGTMVTVSSG | |
| | GGGSGGGGSGGGGSDIQMIQSPSSL | |
| | SASVGDRVTITCRASQTIWSYLNWY | |
| | RQRPGEAPNLLIYAASSLQSGVPSR | |
| | FSGRGSGTDFTLTISSLQAEDFATY | |
| | YCQQSYSIPQTFGQGTKLEIK | |
| 312 | QVQLQQSGPGMVKPSQTLSLTCAIS | Anti-CD22 m971-L7 |
| | GDSVSSNSVAWNWIRQSPSRGLEWL | scFv heavy chain |
| | GRTYYRSTWYNDYAVSMKSRITINP | variable region |
| | DTNKNQFSLQLNSVTPEDTAVYYCA | |
| | REVTGDLEDAFDIWGQGTMVTVS | |
| | S | |
| 313 | GDSVSSNSVA | Anti-CD22 m971-L7 |
| | | scFv heavy chain CDR1 |
| 314 | TYYRSTWYN | Anti-CD22 m971-L7 |
| | | scFv heavy chain CDR2 |
| 306 | AREVTGDLEDAFDI | Anti-CD22 m971-L7 |
| | | scFv heavy chain CDR3 |
| 315 | DIQMIQSPSSLSASVGDRVTITCRAS | Anti-CD22 m971-L7 |
| | QTIWSYLNWYRQRPGEAPNLLIYAAS | scFv light chain variable |
| | SLQSGVPSRFSGRGSGTDFTLTISSL | region |
| | QAEDFATYYCQQSYSIPQTFGQGTK | |
| | LEIK | |
| 308 | QTIWSY | Anti-CD22 m971-L7 |
| | | scFv light chain CDR1 |
| | AAS | Anti-CD22 m971-L7 |
| | | scFv light chain CDR2 |
| 310 | QQSYSIPQT | Anti-CD22 m971-L7 |
| | | scFv light chain CDR3 |

In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280(1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8a hinge domain, for example, a human CD8a hinge domain. In some embodiments, the hinge domain comprises a CD28 hinge domain, for example, a human CD28 hinge domain. In some embodiments, the hinge domain comprises an IgG4 hinge domain, for example, a human IgG4 hinge domain. In some embodiments, the hinge domain comprises a IgG4 hinge-Ch2-Ch3 domain, for example, a human IgG4 hinge-Ch2-Ch3 domain.

In some embodiments, the transmembrane domain of the CD22 CAR comprises a CD8a transmembrane domain, for example, a human CD8a transmembrane domain. In some embodiments, the transmembrane domain comprises a CD28 transmembrane domain, for example, a human CD28 transmembrane domain.

In some embodiments, the intracellular costimulatory domain of the CD22 CAR comprises a 4-1BB costimulatory domain, for example, a human 4-1BB costimulatory domain. In some embodiments, the intracellular costimulatory domain comprises a CD28 costimulatory domain, for example, a human CD28 costimulatory domain.

In some embodiments, the intracellular signaling domain of the CD22 CAR comprises a CD3-zeta (ζ) signaling domain, for example, a human CD3ζ signaling domain.

In some embodiments, the CAR is a CD22 CAR, including, for example, a CD22 CAR comprising a CD22-specific scFv, a CD8a hinge domain, a CD8a transmembrane domain a 4-1BB costimulatory domain, a CD3ζ signaling domain, and/or variants (i.e., having a sequence that is at least about 80% identical, for example, at least about 80%, at least about 85%, at least about 90%, at least about 95%, at least about 96%, at least about 97%, at least about 98%, or at least about 99% identity) thereof. In some embodiments, the first barcode and/or the second barcode is located at the junction of the CD22-specific scFv and the CD8a hinge domain. In some embodiments, the first barcode and/or the second barcode is located at the junction of the CD8a hinge domain and the CD8a transmembrane domain. In some embodiments, the first barcode and/or the second barcode is located at the junction of the CD8a transmembrane domain and the 4-1BB costimulatory domain. In some embodiments, the first barcode and/or the second barcode is located at the junction of the 4-1BB costimulatory domain and the CD3ζ signaling domain.
In some embodiments, the CAR is a CD22 CAR, including, for example, a CD22 CAR comprising a CD22-specific scFv, a CD28 hinge domain, a CD8a transmembrane domain, a 4-1BB costimulatory domain, a CD3ζ signaling domain, and/or variants (i.e., having a sequence that is at least about 80% identical, for example, at least about 80%, at least about 85%, at least about 90%, at least about 95%, at least about 96%, at least about 97%, at least about 98%, or at least about 99% identity) thereof. In some embodiments, the first barcode and/or the second barcode is located at the junction of the CD22-specific scFv and the CD28 hinge domain. In some embodiments, the first barcode and/or the second barcode is located at the junction of the CD28 hinge domain and the CD8a transmembrane domain. In some embodiments, the first barcode and/or the second barcode is located at the junction of the CD8a transmembrane domain and the 4-1BB costimulatory domain. In some embodiments, the first barcode and/or the second barcode is located at the junction of the 4-1BB costimulatory domain and the CD3ζ signaling domain.

In some embodiments, the CAR is a CD22 CAR, including, for example, a CD22 CAR comprising a CD22-specific scFv, an IgG4 hinge domain, a CD8a transmembrane domain, a 4-1BB costimulatory domain, a CD3ζ signaling domain, and/or variants (i.e., having a sequence that is at least about 80% identical, for example, at least about 80%, at least about 85%, at least about 90%, at least about 95%, at least about 96%, at least about 97%, at least about 98%, or at least about 99% identity) thereof. In some embodiments, the first barcode and/or the second barcode is located at the junction of the CD22-specific scFv and the IgG4 hinge domain. In some embodiments, the first barcode and/or the second barcode is located at the junction of the IgG4 hinge domain and the CD8a transmembrane domain. In some embodiments, the first barcode and/or the second barcode is located at the junction of the CD8a transmembrane domain and the 4-1BB costimulatory domain. In some embodiments, the first barcode and/or the second barcode is located at the junction of the 4-1BB costimulatory domain and the CD3ζ signaling domain.

In some embodiments, the CAR is a CD22 CAR, including, for example, a CD22 CAR comprising a CD22-specific scFv, a CD8a hinge domain, a CD28 transmembrane domain, a 4-1BB costimulatory domain, a CD3ζ signaling domain, and/or variants (i.e., having a sequence that is at least about 80% identical, for example, at least about 80%, at least about 85%, at least about 90%, at least about 95%, at least about 96%, at least about 97%, at least about 98%, or at least about 99% identity) thereof. In some embodiments, the first barcode and/or the second barcode is located at the junction of the CD22-specific scFv and the CD8a hinge domain. In some embodiments, the first barcode and/or the second barcode is located at the junction of the CD8a hinge domain and the CD28 transmembrane domain. In some embodiments, the first barcode and/or the second barcode is located at the junction of the CD28 transmembrane domain and the 4-1BB costimulatory domain. In some embodiments, the first barcode and/or the second barcode is located at the junction of the 4-1BB costimulatory domain and the CD3ζ signaling domain.

In some embodiments, the CAR is a CD22 CAR, including, for example, a CD22 CAR comprising a CD22-specific scFv, a CD28 hinge domain, a CD28 transmembrane domain, a 4-1BB costimulatory domain, a CD3ζ signaling domain, and/or variants (i.e., having a sequence that is at least about 80% identical, for example, at least about 80%, at least about 85%, at least about 90%, at least about 95%, at least about 96%, at least about 97%, at least about 98%, or at least about 99% identity) thereof. In some embodiments, the first barcode and/or the second barcode is located at the junction of the CD22-specific scFv and the CD28 hinge domain. In some embodiments, the first barcode and/or the second barcode is located at the junction of the CD28 hinge domain and the CD28 transmembrane domain. In some embodiments, the first barcode and/or the second barcode is located at the junction of the CD28 transmembrane domain and the 4-1BB costimulatory domain. In some embodiments, the first barcode and/or the second barcode is located at the junction of the 4-1BB costimulatory domain and the CD3ζ signaling domain.

In some embodiments, the CAR is a CD22 CAR, including, for example, a CD22 CAR comprising a CD22-specific scFv, an IgG4 hinge domain, a CD28 transmembrane domain, a 4-1BB costimulatory domain, a CD3ζ signaling domain, and/or variants (i.e., having a sequence that is at least about 80% identical, for example, at least about 80%, at least about 85%, at least about 90%, at least about 95%, at least about 96%, at least about 97%, at least about 98%, or at least about 99% identity) thereof. In some embodiments, the first barcode and/or the second barcode is located at the junction of the CD22-specific scFv and the IgG4 hinge domain. In some embodiments, the first barcode and/or the second barcode is located at the junction of the IgG4 hinge domain and the CD28 transmembrane domain. In some embodiments, the first barcode and/or the second barcode is located at the junction of the CD28 transmembrane domain and the 4-1BB costimulatory domain. In some embodiments, the first barcode and/or the second barcode is located at the junction of the 4-1BB costimulatory domain and the CD3ζ signaling domain.

### Exemplary BCMA CARs

In some embodiments, the CAR is a BCMA CAR (“BCMA-CAR”). BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR may comprise an extracellular binding domain that specifically binds BCMA and one or more of: any of the signal peptides described herein, any of the hinge domains described herein, any of the transmembrane domains described herein, any of the intracellular costimulatory domains described herein, and/or any of the intracellular signaling domains described herein, e.g., in tandem.

In some embodiments, the signal peptide of the BCMA CAR comprises a CD8a signal peptide. In some embodiments, the signal peptide comprises an IgK signal peptide. In some embodiments, the signal peptide comprises a GMCSFR-α or CSF2RA signal peptide.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, erlanatamab, teclistamab, LCAR-B38M, or ciltacabtagene. In any of these embodiments, the extracellular binding domain of the BCMA CAR can comprise or consist of the V_{H}, the V_{L}, and/or one or more CDRs of any of the antibodies.

In some embodiments, the extracellular binding domain of the BCMA CAR comprises an scFv derived from C11D5.3, a murine monoclonal antibody as described in Carpenter et al., Clin. Cancer Res. 19(8):2048-2060 (2013). See also PCT Application Publication No. WO2010/104949. The C11D5.3-derived scFv may comprise the heavy chain variable region (V_{H}) and the light chain variable region (V_{L}) of C11D5.3 connected by the Whitlow linker, the amino acid sequence of which is provided in Table 10 below. In some embodiments, the extracellular binding domain of the BCMA CAR comprises an scFv derived from another murine monoclonal antibody, C12A3.2, as described in Carpenter et al., Clin. Cancer Res. 19(8):2048-2060 (2013) and PCT Application Publication No. WO2010/104949, the amino acid sequence of which is also provided in Table 10 below. In some embodiments, the extracellular binding domain of the BCMA CAR comprises a fully human heavy-chain variable domain (FHVH) as described in Lam et al., Nat. Commun. 11(1):283 (2020), also referred to as FHVH33. See also, PCT Application Publication No. WO2019/006072, and Table 10. In some embodiments, the extracellular binding domain of the BCMA CAR comprises an scFv derived from CT103A (or CAR0085) as described in U.S. Pat. No. 11,026,975 B2, the amino acid sequence of which is provided in Table 10 below. In some embodiments, the BCMA-specific extracellular binding domain may comprise one or more CDRs having CDR amino acid sequences set forth in Table 10. In some embodiments, the BCMA-specific extracellular binding domain may comprise a light chain with one or more CDRs having light chain CDR amino acid sequences set forth in Table 10. In some embodiments, the BCMA-specific extracellular binding domain may comprise a heavy chain with one or more CDRs having heavy chain CDR amino acid sequences set forth in Table 10. In any of these embodiments, the BCMA-specific scFv may comprise one or more CDRs comprising one or more amino acid substitutions, or comprising a sequence that is at least about 80% identical (e.g., at least about 80%, at least about 85%, at least about 90%, at least about 95%, at least about 96%, at least about 97%, at least about 98%, at least about 99%, or about 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29(5):585-601 (2018)). See also, PCT Application Publication No. WO2012163805. In some embodiments, the extracellular binding domain of the BCMA CAR comprises single variable fragments of two heavy chains (VHH) that can bind to two epitopes of BCMA as described in Zhao et al., J. Hematol. Oncol. 11(1):141 (2018), also referred to as LCAR-B38M. See also, PCT Application Publication No. WO2018/028647.

Additionally, CARs and binders directed to BCMA have been described in U.S. Application Publication Nos. 2020/0246381 A1 and 2020/0339699 A1, the entire contents of each of which are incorporated by reference herein.

TABLE 10
Exemplary sequences of anti-BCMA binder and components.

| SEQ ID NO: | Amino Acid Sequence | Description |
|---|---|---|
| 316 | DIVLTQSPASLAMSLGKRATISCRAS | Anti-BCMA C11D5.3 |
| | ESVSVIGAHLIHWYQQKPGQPPKLLI | scFv entire sequence, |
| | YLASNLETGVPARFSGSGSGTDFTLT | with Whitlow linker |
| | IDPVEEDDVAIYSCLQSRIFPRTFGG | |
| | GTKLEIKGSTSGSGKPGSGEGSTKGQ | |
| | IQLVQSGPELKKPGETVKISCKASGY | |
| | TFTDYSINWVKRAPGKGLKWMGWINT | |
| | ETREPAYAYDFRGRFAFSLETSASTA | |
| | YLQINNLKYEDTATYFCALDYSYAMD | |
| | YWGQGTSVTVSS | |
| 317 | DIVLTQSPASLAMSLGKRATISCRAS | Anti-BCMA C11D5.3 |
| | ESVSVIGAHLIHWYQQKPGQPPKLLI | scFv light chain variable |
| | YLASNLETGVPARFSGSGSGTDFTLT | region |
| | IDPVEEDDVAIYSCLQSRIFPRTFGG | |
| | GTKLEIK | |
| 318 | RASESVSVIGAHLIH | Anti-BCMA C11D5.3 |
| | | scFv light chain CDR1 |
| 319 | LASNLET | Anti-BCMA C11D5.3 |
| | | scFv light chain CDR2 |
| 320 | LQSRIFPRT | Anti-BCMA C11D5.3 |
| | | scFv light chain CDR3 |
| 321 | QIQLVQSGPELKKPGETVKISCKASG | Anti-BCMA C11D5.3 |
| | YTFTDYSINWVKRAPGKGLKWMG | scFv heavy chain |
| | WINTETREPAYAYDFRGRFAFSLETS | variable region |
| | ASTAYLQINNLKYEDTATYFCALDY | |
| | SYAMDYWGQGTSVTVSS | |
| 322 | DYSIN | Anti-BCMA C11D5.3 |
| | | scFv heavy chain CDR1 |
| 323 | WINTETREPAYAYDFRG | Anti-BCMA C11D5.3 |
| | | scFv heavy chain CDR2 |
| 324 | DYSYAMDY | Anti-BCMA C11D5.3 |
| | | scFv heavy chain CDR3 |
| 325 | DIVLTQSPPSLAMSLGKRATISCRAS | Anti-BCMA C12A3.2 |
| | ESVTILGSHLIYWYQQKPGQPPTLLI | scFv entire sequence, |
| | QLASNVQTGVPARFSGSGSRTDFTL | with Whitlow linker |
| | TIDPVEEDDVAVYYCLQSRTIPRTFG | |
| | GGTKLEIKGSTSGSGKPGSGEGSTK | |
| | GQIQLVQSGPELKKPGETVKISCKAS | |
| | GYTFRHYSMNWVKQAPGKGLKWM | |
| | GRINTESGVPIYADDFKGRFAFSVET | |
| | SASTAYLVINNLKDEDTASYFCSND | |
| | YLYSLDFWGQGTALTVSS | |
| 326 | DIVLTQSPPSLAMSLGKRATISCRAS | Anti-BCMA C12A3.2 |
| | ESVTILGSHLIYWYQQKPGQPPTLLI | scFv light chain variable |
| | QLASNVQTGVPARFSGSGSRTDFTL | region |
| | TIDPVEEDDVAVYYCLQSRTIPRTFG | |
| | GGTKLEIK | |
| 327 | RASESVTILGSHLIY | Anti-BCMA C12A3.2 |
| | | scFv light chain CDR1 |
| 328 | LASNVQT | Anti-BCMA C12A3.2 |
| | | scFv light chain CDR2 |
| 329 | LQSRTIPRT | Anti-BCMA C12A3.2 |
| | | scFv light chain CDR3 |
| 330 | QIQLVQSGPELKKPGETVKISCKASG | Anti-BCMA C12A3.2 |
| | YTFRHYSMNWVKQAPGKGLKWMG | scFv heavy chain |
| | RINTESGVPIYADDFKGRFAFSVETS | variable region |
| | ASTAYLVINNLKDEDTASYFCSNDY | |
| | LYSLDFWGQGTALTVSS | |
| 331 | HYSMN | Anti-BCMA C12A3.2 |
| | | scFv heavy chain CDR1 |
| 332 | RINTESGVPIYADDFKG | Anti-BCMA C12A3.2 |
| | | scFv heavy chain CDR2 |
| 333 | DYLYSLDF | Anti-BCMA C12A3.2 |
| | | scFv heavy chain CDR3 |
| 334 | EVQLLESGGGLVQPGGSLRLSCAAS | Anti-BCMA FHVH33 |
| | GFTFSSYAMSWVRQAPGKGLEWVSS | entire sequence |
| | ISGSGDYIYYADSVKGRFTISRDIS | |
| | KNTLYLQMNSLRAEDTAVYYCAKEG | |
| | TGANSSLADYRGQGTLVTVSS | |
| 335 | GFTFSSYA | Anti-BCMA FHVH33 |
| | | CDR1 |
| 336 | ISGSGDYI | Anti-BCMA FHVH33 |
| | | CDR2 |
| 337 | AKEGTGANSSLADY | Anti-BCMA FHVH33 |
| | | CDR3 |
| 338 | DIQMTQSPSSLSASVGDRVTITCRAS | Anti-BCMA CT103A |
| | QSISSYLNWYQQKPGKAPKLLIYAA | scFv entire sequence, |
| | SSLQSGVPSRFSGSGSGTDFTLTISSL | with Whitlow linker |
| | QPEDFATYYCQQKYDLLTFGGGTK | |
| | VEIKGSTSGSGKPGSGEGSTKGQLQ | |
| | LQESGPGLVKPSETLSLTCTVSGGSI | |
| | SSSSYYWGWIRQPPGKGLEWIGSISY | |
| | SGSTYYNPSLKSRVTISVDTSKNQFS | |
| | LKLSSVTAADTAVYYCARDRGDTIL | |
| | DVWGQGTMVTVSS | |
| 339 | DIQMTQSPSSLSASVGDRVTITCRAS | Anti-BCMA CT103A |
| | QSISSYLNWYQQKPGKAPKLLIYAA | scFv light chain variable |
| | SSLQSGVPSRFSGSGSGTDFTLTISSL | region |
| | QPEDFATYYCQQKYDLLTFGGGTK | |
| | VEIK | |
| 340 | QSISSY | Anti-BCMA CT103A |
| | | scFv light chain CDR1 |
| | AAS | Anti-BCMA CT103A |
| | | scFv light chain CDR2 |
| 341 | QQKYDLLT | Anti-BCMA CT103A |
| | | scFv light chain CDR3 |
| 342 | QLQLQESGPGLVKPSETLSLTCTVSG | Anti-BCMA CT103A |
| | GSISSSSYYWGWIRQPPGKGLEWIGS | scFv heavy chain |
| | ISYSGSTYYNPSLKSRVTISVDTSKN | variable region |
| | QFSLKLSSVTAADTAVYYCARDRG | |
| | DTILDVWGQGTMVTVSS | |
| 343 | GGSISSSSYY | Anti-BCMA CT103A |
| | | scFv heavy chain CDR1 |
| 344 | ISYSGST | Anti-BCMA CT103A |
| | | scFv heavy chain CDR2 |
| 345 | ARDRGDTILDV | Anti-BCMA CT103A |
| | | scFv heavy chain CDR3 |
| 346 | atggccttaccagtgaccgccttgctcctgccgctggcct | Exemplary BCMA CAR |
| | tgctgctccacgccgccaggccggacatccagatgacc | nucleotide sequence |
| | cagtctccatcctccctgtctgcatctgtaggagacagag | |
| | tcaccatcacttgccgggcaagtcagagcattagcagct | |
| | atttaaattggtatcagcagaaaccagggaaagccccta | |
| | agctcctgatctatgctgcatccagtttgcaaagtggggt | |
| | cccatcaaggttcagtggcagtggatctgggacagattt | |
| | cactctcaccatcagcagtctgcaacctgaagattttgca | |
| | acttactactgtcagcaaaaatacgacctcctcacttttgg | |
| | cggagggaccaaggttgagatcaaaggcagcaccagc | |
| | ggctccggcaagcctggctctggcgagggcagcacaa | |
| | agggacagctgcagctgcaggagtcgggcccaggact | |
| | ggtgaagccttcggagaccctgtccctcacctgcactgt | |
| | ctctggtggctccatcagcagtagtagttactactggggc | |
| | tggatccgccagcccccagggaaggggctggagtgga | |
| | ttgggagtatctcctatagtgggagcacctactacaaccc | |
| | gtccctcaagagtcgagtcaccatatccgtagacacgtc | |
| | caagaaccagttctccctgaagctgagttctgtgaccgc | |
| | cgcagacacggcggtgtactactgcgccagagatcgtg | |
| | gagacaccatactagacgtatggggtcagggtacaatg | |
| | gtcaccgtcagctcattcgtgcccgtgttcctgcccgcca | |
| | aacctaccaccacccctgcccctagacctcccacccca | |
| | gccccaacaatcgccagccagcctctgtctctgcggcc | Exemplary BCMA CAR |
| | cgaagcctgtagacctgctgccggcggagccgtgcac | |
| | accagaggcctggacttcgcctgcgacatctacatctgg | |
| | gcccctctggccggcacctgtggcgtgctgctgctgag | |
| | cctggtgatcaccctgtactgcaaccaccggaacaaac | |
| | ggggcagaaagaaactcctgtatatattcaaacaaccatt | |
| | tatgagaccagtacaaactactcaagaggaagatggct | |
| | gtagctgccgatttccagaagaagaagaaggaggatgt | |
| | gaactgagagtgaagttcagcagatccgccgacgcccc | |
| | tgcctaccagcagggacagaaccagctgtacaacgag | |
| | ctgaacctgggcagacgggaagagtacgacgtgctgg | |
| | acaagcggagaggccgggaccccgagatgggcgga | |
| | aagcccagacggaagaacccccaggaaggcctgtata | |
| | acgaactgcagaaagacaagatggccgaggcctacag | |
| | cgagatcggcatgaagggcgagcggaggcgcggcaa | |
| | gggccacgatggcctgtaccagggcctgagcaccgcc | |
| | accaaggacacctacgacgccctgcacatgcaggccct | |
| | gccccccaga | |
| 347 | MALPVTALLLPLALLLHAARPDIQM | amino acid sequence |
| | TQSPSSLSASVGDRVTITCRASQSISS | |
| | YLNWYQQKPGKAPKLLIYAASSLQS | |
| | GVPSRFSGSGSGTDFTLTISSLQPEDF | |
| | ATYYCQQKYDLLTFGGGTKVEIKGS | |
| | TSGSGKPGSGEGSTKGQLQLQESGP | |
| | GLVKPSETLSLTCTVSGGSISSSSYY | |
| | WGWIRQPPGKGLEWIGSISYSGSTY | |
| | YNPSLKSRVTISVDTSKNQFSLKLSS | |
| | VTAADTAVYYCARDRGDTILDVWG | |
| | QGTMVTVSSFVPVFLPAKPTTTPAP | |
| | RPPTPAPTIASQPLSLRPEACRPAAG | |
| | GAVHTRGLDFACDIYIWAPLAGTCG | |
| | VLLLSLVITLYCNHRNKRGRKKLLY | |
| | IFKQPFMRPVQTTQEEDGCSCRFPEE | |
| | EEGGCELRVKFSRSADAPAYQQGQ | |
| | NQLYNELNLGRREEYDVLDKRRGR | |
| | DPEMGGKPRRKNPQEGLYNELQKD | |
| | KMAEAYSEIGMKGERRRGKGHDGL | |
| | YQGLSTATKDTYDALHMQALPPR | |

In some embodiments, the hinge domain of the BCMA CAR comprises a CD8α hinge domain, for example, a human CD8α hinge domain. In some embodiments, the hinge domain comprises a CD28 hinge domain, for example, a human CD28 hinge domain. In some embodiments, the hinge domain comprises an IgG4 hinge domain, for example, a human IgG4 hinge domain. In some embodiments, the hinge domain comprises a IgG4 hinge-Ch2-Ch3 domain, for example, a human IgG4 hinge-Ch2-Ch3 domain.

In some embodiments, the transmembrane domain of the BCMA CAR comprises a CD8a transmembrane domain, for example, a human CD8a transmembrane domain. In some embodiments, the transmembrane domain comprises a CD28 transmembrane domain, for example, a human CD28 transmembrane domain.

In some embodiments, the intracellular costimulatory domain of the BCMA CAR comprises a 4-1BB costimulatory domain, for example, a human 4-1BB costimulatory domain. In some embodiments, the intracellular costimulatory domain comprises a CD28 costimulatory domain, for example, a human CD28 costimulatory domain.

In some embodiments, the intracellular signaling domain of the BCMA CAR comprises a CD3 zeta (ζ) signaling domain, for example, a human CD3ζ signaling domain.

In some embodiments, the CAR is a BCMA CAR, including, for example, a BCMA CAR comprising any of the BCMA-specific extracellular binding domains as described, a CD8a hinge domain, a CD8a transmembrane domain, a 4-1BB costimulatory domain, a CD3ζ signaling domain, and/or variants (i.e., having a sequence that is at least about 80% identical, for example, at least about 80%, at least about 85%, at least about 90%, at least about 95%, at least about 96%, at least about 97%, at least about 98%, or at least about 99% identity) thereof. In any of these embodiments, the BCMA CAR may additionally comprise a signal peptide (e.g., a CD8a signal peptide) as described above. In some embodiments, the first barcode and/or the second barcode is located at the junction of the BCMA-specific extracellular binding domain and the CD8a hinge domain. In some embodiments, the first barcode and/or the second barcode is located at the junction of the CD8a hinge domain and the CD8a transmembrane domain. In some embodiments, the first barcode and/or the second barcode is located at the junction of the CD8a transmembrane domain and the 4-1BB costimulatory domain. In some embodiments, the first barcode and/or the second barcode is located at the junction of the 4-1BB costimulatory domain and the CD3ζ signaling domain.

In some embodiments, the CAR is a BCMA CAR, including, for example, a BCMA CAR comprising any of the BCMA-specific extracellular binding domains as described, a CD8a hinge domain, a CD8a transmembrane domain, a CD28 costimulatory domain, a CD3ζ signaling domain, and/or variants (i.e., having a sequence that is at least about 80% identical, for example, at least about 80%, at least about 85%, at least about 90%, at least about 95%, at least about 96%, at least about 97%, at least about 98%, or at least about 99% identity) thereof. In any of these embodiments, the BCMA CAR may additionally comprise a signal peptide as described above. In some embodiments, the first barcode and/or the second barcode is located at the junction of the BCMA-specific extracellular binding domain and the CD8a hinge domain. In some embodiments, the first barcode and/or the second barcode is located at the junction of the CD8a hinge domain and the CD8a transmembrane domain. In some embodiments, the first barcode and/or the second barcode is located at the junction of the CD8a transmembrane domain and the CD28 costimulatory domain. In some embodiments, the first barcode and/or the second barcode is located at the junction of the CD28 costimulatory domain and the CD3ζ signaling domain.

In some embodiments, the CAR is a BCMA CAR as set forth in SEQ ID NO:346 or is at least about 80% identical (e.g., at least about 80%, at least about 85%, at least about 90%, at least about 95%, at least about 96%, at least about 97%, at least about 98%, at least about 99%, or about 100% identical) thereto (see Table 10). The encoded BCMA CAR has a corresponding amino acid sequence set forth in SEQ ID NO:347 or is at least about 80% identical (e.g., at least about 80%, at least about 85%, at least about 90%, at least about 95%, at least about 96%, at least about 97%, at least about 98%, at least about 99%, or about 100% identical) thereto, with the following components: CD8a signal peptide, CT103A scFv (V_{L}—Whitlow linker-V_{H}), CD8a hinge domain, CD8a transmembrane domain, 4-1BB costimulatory domain, and CD3ζ signaling domain. In some embodiments, the first barcode and/or the second barcode is located at the junction of the CT103A scFv and the CD8a hinge domain. In some embodiments, the first barcode and/or the second barcode is located at the junction of the CD8a hinge domain and the CD8a transmembrane domain. In some embodiments, the first barcode and/or the second barcode is located at the junction of the CD8a transmembrane domain and the 4-1BB costimulatory domain. In some embodiments, the first barcode and/or the second barcode is located at the junction of the 4-1BB costimulatory domain and the CD3ζ signaling domain.

In some embodiments, the CAR is a commercially available embodiment of BCMA CAR, including, for example, idecabtagene vicleucel (ide-cel, also called bb2121). In some embodiments, the CAR is idecabtagene vicleucel or portions thereof. Idecabtagene vicleucel comprises a BCMA CAR with the following components: the BB2121 binder, CD8a hinge domain, CD8a transmembrane domain, 4-1BB costimulatory domain, and CD3ζ signaling domain.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

For example, in some embodiments, any of the CARs herein contain an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3-zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-IBB or functional variant thereof and a signaling portion of CD3-zeta or functional variant thereof. In some such embodiments, the receptor further includes a spacer containing a portion of an Ig molecule, such as a human Ig molecule, such as an Ig hinge, e.g., an IgG4 hinge, such as a hinge-only spacer.

In some aspects, the spacer contains only a hinge region of an IgG, such as only a hinge of IgG4 or IgG1. In other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains. In some embodiments, the spacer is an Ig hinge, e.g., an IgG4 hinge, linked to a CH3 domain only. In some embodiments, the spacer is or comprises a glycine-serine rich sequence or other flexible linker such as known flexible linkers.

For example, in some embodiments, any of the CARs herein include an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3-zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3-zeta-derived signaling domain.

The recombinant receptors, such as any of the CARs of the disclosure, expressed by cells administered to a subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition. In any of these embodiments, the extracellular binding domain of the CAR can be codon-optimized for expression in a host cell or have variant sequences to increase functions of the extracellular binding domain.

b. T Cell Receptors

In some embodiments, the one or more transgenes comprise a T cell receptor (TCR). In some embodiments, the transgene encodes a T cell receptor (TCR) or antigen-binding portion thereof that recognizes a peptide epitope or T cell epitope of a target polypeptide, such as an antigen of a tumor, viral or autoimmune protein.
In some embodiments, a T cell receptor or TCR is a molecule that contains a variable a and b chain (also known as TCRalpha and TCRbeta, respectively) or a variable g and d chains (also known as TCRalpha and TCRbeta, respectively), or antigen-binding portions thereof, and which is capable of specifically binding to a peptide bound to an MHC molecule. In some embodiments, the TCR is in the alpha-beta (α-β) form. Typically, TCRs that exist in alpha-beta and gamma-delta forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the α-β form or γ-δ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable a chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

c. Chimeric Auto-Antibody Receptors (CAAR) and B-cell Autoantibody Receptors (BAR)

In some embodiments, the one or more transgenes comprise a chimeric autoantibody receptor (CAAR).

In some embodiments, the CAAR binds, e.g., specifically binds, or recognizes, an autoantibody. In some embodiments, a cell expressing the CAAR, such as a T cell engineered to express a CAAR, can be used to bind to and kill autoantibody-expressing cells, but not normal antibody expressing cells. In some embodiments, CAAR-expressing cells can be used to treat an autoimmune disease associated with expression of self-antigens, such as autoimmune diseases. In some embodiments, CAAR-expressing cells can target B cells that ultimately produce the autoantibodies and display the autoantibodies on their cell surfaces, mark these B cells as disease-specific targets for therapeutic intervention. In some embodiments, CAAR-expressing cells can be used for efficiently targeting and killing the pathogenic B cells in autoimmune diseases by targeting the disease-causing B cells using an antigen-specific chimeric autoantibody receptor. In some embodiments, the recombinant receptor is a CAAR, such as any described in U.S. Patent Application Pub. No. US 2017/0051035.

In some embodiments, the CAAR comprises an antigen selected from a pancreatic β-cell antigen, synovial joint antigen, myelin basic protein, proteolipid protein, myelin oligodendritic glycoprotein, MuSK, keratinocyte adhesion protein desmoglein 3 (Dsg3), Ro-RNP complex, La antigen, myeloperoxidase, proteinase 3, cardiolipin, citrullinated proteins, carbamylated proteins, α3 chain of basement membrane collagen, or any combination thereof.

In some embodiments, the CAAR comprises an autoantibody binding domain, a transmembrane domain, and one or more intracellular signaling regions or domains (also interchangeably called a cytoplasmic signaling domain or region). In some embodiments, the intracellular signaling region comprises an intracellular signaling domain. In some embodiments, the intracellular signaling domain is or comprises a primary signaling domain, a signaling domain that is capable of stimulating and/or inducing a primary activation signal in a T cell, a signaling domain of a T cell receptor (TCR) component (e.g., an intracellular signaling domain or region of a CD3-zeta) chain or a functional variant or signaling portion thereof), and/or a signaling domain comprising an immunoreceptor tyrosine-based activation motif (ITAM).

In some embodiments, the one or more transgenes comprise a B-cell autoantibody receptor (BAR). In some embodiments, the BAR comprises an FVIII antigen.
In some embodiments, the autoantibody binding domain comprises an autoantigen or a fragment thereof. The choice of autoantigen can depend upon the type of autoantibody being targeted. For example, the autoantigen may be chosen because it recognizes an autoantibody on a target cell, such as a B cell, associated with a particular disease state, e.g., an autoimmune disease, such as an autoantibody-mediated autoimmune disease. In some embodiments, the autoimmune disease includes pemphigus vulgaris (PV). Exemplary autoantigens include desmoglein 1 (Dsg1) and Dsg3.

In some embodiments, the first transgene and/or the second transgene encodes an antibody or an antibody fragment, a chimeric antigen receptor (CAR), a chimeric autoantibody receptor (CAAR), a B-cell autoantibody receptor (BAR), a T cell receptor (TCR), or one or more tolerogenic factors. In some embodiments, the first transgene and/or the second transgene encodes a CAR.

In some embodiments, the CAR encoded by the first transgene and/or the second transgene comprises a hinge domain, a transmembrane domain, and one or more signaling domains. In some embodiments, the hinge domain is a hinge domain of a naturally occurring protein. Hinge domains of any protein known in the art to comprise a hinge domain are compatible for use in the CARs as described herein. In some embodiments, the hinge domain is at least a portion of a hinge domain of a naturally occurring protein and confers flexibility to the CAR as described herein. In some embodiments, the hinge domain is a variant of a hinge domain of a naturally occurring protein (i.e., having a sequence that is at least about 80% identical, for example, at least about 80%, at least about 85%, at least about 90%, at least about 95%, at least about 96%, at least about 97%, at least about 98%, or at least about 99% identity).

In some embodiments, the hinge domain is selected from the group consisting of: CD8a hinge domain, CD28 hinge domain, IgG4 hinge domain, IgG4 hinge-CH2-CH3 domain, and any functional variant thereof. In some embodiments, the hinge domain is derived from CD8a. In some embodiments, the hinge domain is a portion of the hinge domain of CD8a, or any functional variant thereof. In some embodiments, the hinge domain is derived from CD28. In some embodiments, the hinge domain is a portion of the hinge domain of CD28, or any functional variant thereof. In some embodiments, the hinge domain is derived from IgG4. In some embodiments, the hinge domain is a portion of the hinge domain of IgG4, or any functional variant thereof. In some embodiments, the hinge domain is derived from IgG4 hinge-CH2-CH3. In some embodiments, the hinge domain is a portion of the hinge domain of IgG4 hinge-CH2-CH3, or any functional variant thereof.
In some embodiments, the transmembrane domain is selected from the group consisting of: alpha, beta, or zeta chain of a T cell receptor, CD28, CD3ε, CD45, CD4, CD5, CD8, CD9, CD16, CD22, CD33, CD3γ, CD64, CD80, CD86, CD134, CD137, CD154, CD8a, CD80, 4-1BB/CD137, CD28, CD34, CD4, FcεRIγ, CD16, OX40/CD134, CD3ζ, CD3δ, CD3γ, CD3δ, TCRα, TCRβ, TCRζ, CD32, CD64, CD64, CD45, CD5, CD9, CD22, CD3γ, CD80, CD86, CD40, CD40L/CD154, VEGFR2, FAS, FGFR2B, and any functional variant thereof. In some embodiments, the signaling domain is selected from the group consisting of: B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3ζ, an immunoreceptor tyrosine-based activation motif (ITAM), CD27, 4-1BB, CD134/OX40, CD30, CD40, PD-1, ICOS, LIGHT, NKG2C, a ligand that specifically binds with CD83, any functional variant thereof, and any combination thereof.
In some embodiments, the CAR encoded by the first transgene and/or the second transgene comprises a CD8a hinge domain, a CD8a transmembrane domain, a 4-1BB domain, and a CD3zeta domain. In some embodiments, the CAR encoded by the first transgene and/or the second transgene is selected from the group consisting of: a CD5-specific CAR, a CD19-specific CAR, a CD20-specific CAR, a CD22-specific CAR, a CD23-specific CAR, a CD30-specific CAR, a CD33-specific CAR, CD38-specific CAR, a CD70-specific CAR, a CD123-specific CAR, a CD138-specific CAR, a Kappa, Lambda, B cell maturation agent (BCMA)-specific CAR, a G-protein coupled receptor family C group 5 member D (GPRC5D)-specific CAR, a CD123-specific CAR, a LeY-specific CAR, a NKG2D ligand-specific CAR, a WT1-specific CAR, a GD2-specific CAR, a HER2-specific CAR, a EGFR-specific CAR, a EGFRvIII-specific CAR, a B7H3-specific CAR, a PSMA-specific CAR, a PSCA-specific CAR, a CAIX-specific CAR, a CD171-specific CAR, a CEA-specific CAR, a CSPG4-specific CAR, a EPHA2-specific CAR, a FAP-specific CAR, a FRa-specific CAR, a IL-13Rα-specific CAR, a Mesothelin-specific CAR, a MUC1-specific CAR, a MUC16-specific CAR, a ROR1-specific CAR, a C-Met-specific CAR, a CD133-specific CAR, a Ep-CAM-specific CAR, a GPC3-specific CAR, a HPV16-E6-specific CAR, a IL13Ra2-specific CAR, a MAGEA3-specific CAR, a MAGEA4-specific CAR, a MART1-specific CAR, a NY-ESO-1-specific CAR, a VEGFR2-specific CAR, a α-Folate receptor-specific CAR, a CD24-specific CAR, a CD44v7/8-specific CAR, a EGP-2-specific CAR, a EGP-40-specific CAR, a erb-B2-specific CAR, a erb-B 2,3,4-specific CAR, a FBP-specific CAR, a Fetal acethylcholine e receptor-specific CAR, a GD2-specific CAR, a GD3-specific CAR, a HMW-MAA-specific CAR, a IL-11Rα-specific CAR, a KDR-specific CAR, a Lewis Y-specific CAR, a L1-cell adhesion molecule-specific CAR, a MAGE-A1-specific CAR, a Oncofetal antigen (h5T4)-specific CAR, a TAG-72-specific CAR, and a CD19/CD22-bispecific CAR. In some embodiments, the CAR encoded by the first transgene and/or the second transgene is a CD19 CAR or a CD22 CAR. In some embodiments, the CAR encoded by the first transgene and/or the second transgene further comprises one or more co-stimulatory domain(s).

In some embodiments, the diverged nucleotide sequence within the transgene is located at the junction of: (i) the signaling domain and the co-stimulatory domain; or (ii) the hinge domain and the transmembrane domain. In some embodiments, the diverged nucleotide sequence within the transgene is located at the junction of the 4-1BB and CD3-zeta domains.

v. Tolerogenic Factors

In some embodiments, the one or more transgenes comprise a tolerogenic factor. Tolerogenic factors include any factors that promote or contribute to promoting or inducing tolerance to an engineered cell or population of cells (e.g., a hypoimmunogenic islet cell such as a hypoimmunogenic beta cell) of the disclosure by the immune system (e.g., innate or adaptive immune system). In some embodiments, the expression of a detection agent acts as a signal for the administration of an antibody directed against or specific to a tolerogenic agent or kill switch, e.g., an anti-CD47 antibody. In some embodiments, the kill switch may be an exogenously administered agent that recognizes one or more tolerogenic factor on the surface of the modified cells. In some embodiments, the exogenously administered agent is an antibody directed against or specific to a tolerogenic agent, e.g., an anti-CD47 antibody. By recognizing and blocking a tolerogenic factor on modified cells, an exogenously administered antibody may block the immune inhibitory functions of the tolerogenic factor thereby re-sensitizing the immune system to the modified cells. For instance, for modified cells that overexpresses CD47, an exogenously administered anti-CD47 antibody may be administered to the subject, resulting in masking of CD47 on the modified cells and triggering of an immune response to the modified cells. In some embodiments, the anti-CD47 antibody is Magrolimab.

In some embodiments, the tolerogenic factor comprises one or more of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, DUX4, MANF, or any combinations, functional fragments, or variants thereof. In some embodiments, the tolerogenic factor is CD47, PD-L1, HLA-E or HLA-G, CCL21, FasL, Serpinb9, CD200, Mfge8, or any combinations, functional fragments, or variants thereof. In some embodiments, expression of the tolerogenic (e.g., immune) factor affects immune recognition and tolerance of an engineered cell or population of cells (e.g., T cells, B cells, NK cells, islet cells such as beta cells, or hypoimmunogenic cells thereof) of the disclosure in a recipient. In some embodiments, the engineered cell or population of cells contains an exogenous sequence that encodes the one or more tolerogenic factors. In some embodiments, the tolerogenic factor is overexpressed in the cell. In some embodiments, the expression of the tolerogenic factor is overexpressed or increased in the engineered cell or population of cells, e.g., compared to a similar cell of the same cell type that has not been engineered with the modification, such as a reference or non-engineered cell, e.g., a cell not engineered with a transgene encoding the tolerogenic factor.

In some embodiments, the tolerogenic factor is CD47, or a functional fragment or variant thereof. CD47 is a leukocyte surface antigen and has a role in cell adhesion and modulation of integrins. It is normally expressed on the surface of a cell and signals to circulating macrophages not to eat the cell. In some embodiments, the engineered cell or population of cells (e.g., T cells, B cells, NK cells, islet cells such as beta cells, or hypoimmunogenic cells thereof) contains a transgene sequence that encodes CD47, such as human CD47, or a functional fragment or variant thereof. In some embodiments, CD47, or a functional fragment or variant thereof, is overexpressed in the cell. In some embodiments, the expression of CD47, or a functional fragment or variant thereof, is overexpressed or increased in the engineered cell or population of cells compared to a similar cell of the same cell type that has not been engineered, such as a reference or non-engineered cell, e.g., a cell not engineered with a transgene encoding CD47. In some embodiments, the engineered cell or population of cells contains an overexpressed transgene that encodes CD47, or a functional fragment or variant thereof, such as human CD47. Useful genomic, polynucleotide and polypeptide information about human CD47 are provided in, for example, the NP_001768.1, NP_942088.1, NM_001777.3 and NM_198793.2.

In some embodiments, the cell outlined herein comprises a transgene sequence encoding a CD47 polypeptide that has at least 95% sequence identity (e.g., 95%, 96%, 97%, 98%, 99%, or more) to an amino acid sequence as set forth in NCBI Ref. Sequence Nos. NP_001768.1 and NP_942088.1, or to fragments thereof. In some embodiments, the cell outlined herein comprises a transgene sequence encoding a CD47 polypeptide having an amino acid sequence as set forth in NCBI Ref. Sequence Nos. NP_001768.1 and NP_942088.1, or a functional fragment or variant thereof. In some embodiments, the cell comprises a transgene sequence encoding a CD47 polynucleotide having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2, or to fragments thereof. In some embodiments, the cell comprises a transgene sequence encoding a CD47 polynucleotide as set forth in NCBI Ref. Sequence Nos. NM_001777.3 and NM_198793.2, or fragments thereof.

In some embodiments, the cell outlined herein comprises a transgene sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1, or to fragments thereof. In some embodiments, the cell outlined herein comprises a transgene sequence encoding a CD47 polypeptide having an amino acid sequence as set forth in NCBI Ref. Sequence Nos. NP_001768.1 and NP_942088.1, or a functional fragment or variant thereof. In some embodiments, the cell comprises a transgene sequence encoding a CD47 polynucleotide having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2, or to a functional fragment thereof. In some embodiments, the cell comprises a transgene sequence encoding a CD47 polynucleotide as set forth in NCBI Ref. Sequence Nos. NM_001777.3 and NM_198793.2, or fragments thereof.

In some embodiments, the cell comprises a CD47 polypeptide having at least 95% sequence identity (e.g., 95%, 96%, 97%, 98%, 99%, or more) to an amino acid sequence as set forth in NCBI Ref. Sequence Nos. NP_001768.1 and NP_942088.1, or a functional fragment thereof. In some embodiments, the cell outlined herein comprises a CD47 polypeptide having an amino acid sequence as set forth in NCBI Ref. Sequence Nos. NP_001768.1 and NP_942088.1, or a functional fragment or variant thereof.

In some embodiments, the cell comprises a CD47 polypeptide having at least 95% sequence identity (e.g., 95%, 96%, 97%, 98%, 99%, or more) to an amino acid sequence selected from the group consisting of SEQ ID NOs:1-5, or a functional fragment thereof. In some embodiments, the cell outlined herein comprises a CD47 polypeptide having an amino acid sequence selected from the group consisting of SEQ ID NOs:1-5, or a functional fragment or variant thereof, as laid out in Table 11 below.

TABLE 11
CD47 polypeptide sequences.

| SEQ ID NO: | Description | Amino Acid Sequence |
|---|---|---|
| 1 | CD47-201 protein | MWPLVAALLLGSACCGSAQLLFNKTKSVEFTFCN |
| | | DTVVIPCFVTNMEAQNTTEVYVKWKFKGRDIYTF |
| | | DGALNKSTVPTDFSSAKIEVSQLLKGDASLKMDK |
| | | SDAVSHTGNYTCEVTELTREGETIIELKYRVVSWF |
| | | SPNENILIVIFPIFAILLFWGQFGIKTLKYRSGGMDE |
| | | KTIALLVAGLVITVIVIVGAILFVPGEYSLKNATGL |
| | | GLIVTSTGILILLHYYVFSTAIGLTSFVIAILVIQVIA |
| | | YILAVVGLSLCIAACIPMHGPLLISGLSILALAQLL |
| | | GLVYMKFVASNQKTIQPPRNN |
| 2 | CD47-202 protein- | MWPLVAALLLGSACCGSAQLLENKTKSVEFTFCN |
| | full-length protein | DTVVIPCFVTNMEAQNTTEVYVKWKFKGRDIYTF |
| | | DGALNKSTVPTDFSSAKIEVSQLLKGDASLKMDK |
| | | SDAVSHTGNYTCEVTELTREGETIIELKYRVVSWF |
| | | SPNENILIVIFPIFAILLFWGQFGIKTLKYRSGGMDE |
| | | KTIALLVAGLVITVIVIVGAILFVPGEYSLKNATGL |
| | | GLIVTSTGILILLHYYVFSTAIGLTSFVIAILVIQVIA |
| | | YILAVVGLSLCIAACIPMHGPLLISGLSILALAQLL |
| | | GLVYMKFVASNQKTIQPPRKAVEEPLNAFKESKG |
| | | MMNDE |
| 3 | CD47-203 protein | WTESLYCGVYTNAWPSSDFRFEYLSSSTITWTSLY |
| | | EICGVQRDTYSQLKGKKRQASNQKTIQPPRKAVE |
| | | EPLNAFKESKGMMNDE |
| 4 | CD47-205 protein | KNATGLGLIVTSTGILILLHYYVFSTAIGLTSFVIAI |
| | | LVIQVIAYILAVVGLSLCIAACIPMHGPLLISGLSIL |
| | | ALAQLLGLVYMKFVASNQKTIQPPRKA VEEPLNE |
| 5 | CD47-206 protein | MEAQNTTEVYVKWKFKGRDIYTFDGALNKSTVP |
| | | TDFSSAKIEVSQLLKGDASLKMDKSDAVSHTGNY |
| | | TCEVTELTREGETIIELKYRVVSWFSPNENILIVIFP |
| | | IFAILLFWGQFGIKTLKYRSGGMDEKTIALLVAGL |
| | | VITVIVIVGAILFVPGEYSLKNATGLGLIVTSTGILI |
| | | LLHYYVF |

In some embodiments, the cell comprises a CD47 polypeptide encoded by a nucleic acid sequence having at least 95% sequence identity (e.g., 95%, 96%, 97%, 98%, 99%, or more) to a nucleic acid sequence selected from the group consisting of SEQ ID NOs:6-11, or a functional fragment thereof. In some embodiments, the cell outlined herein comprises a CD47 polypeptide encoded by a nucleic acid sequence having a nucleic acid sequence selected from the group consisting of SEQ ID NOs:6-11, or a functional fragment or variant thereof, as laid out in Table 12 below.

TABLE 12
CD47 nucleic acid sequences.

| SEQ ID NO: | Description | Sequence |
|---|---|---|
| 6 | CD47-201 | ATGTGGCCCCTGGTAGCGGCGCTGTTGCTGGGC |
| | (ENST00000355354.13) | TCGGCGTGCTGCGGATCAGCTCAGCTACTATTT |
| | cDNA | AATAAAACAAAATCTGTAGAATTCACGTTTTGT |
| | | AATGACACTGTCGTCATTCCATGCTTTGTTACT |
| | | AATATGGAGGCACAAAACACTACTGAAGTATAC |
| | | GTAAAGTGGAAATTTAAAGGAAGAGATATTTA |
| | | CACCTTTGATGGAGCTCTAAACAAGTCCACTGT |
| | | CCCCACTGACTTTAGTAGTGCAAAAATTGAAGT |
| | | CTCACAATTACTAAAAGGAGATGCCTCTTTGAA |
| | | GATGGATAAGAGTGATGCTGTCTCACACACAG |
| | | GAAACTACACTTGTGAAGTAACAGAATTAACCA |
| | | GAGAAGGTGAAACGATCATCGAGCTAAAATATC |
| | | GTGTTGTTTCATGGTTTTCTCCAAATGAAAATA |
| | | TTCTTATTGTTATTTTCCCAATTTTTGCTATAC |
| | | TCCTGTTCTGGGGACAGTTTGGTATTAAAACAC |
| | | TTAAATATAGATCCGGTGGTATGGATGAGAAAA |
| | | CAATTGCTTTACTTGTTGCTGGACTAGTGATCA |
| | | CTGTCATTGTCATTGTTGGAGCCATTCTTTTCG |
| | | TCCCAGGTGAATATTCATTAAAGAATGCTACTG |
| | | GCCTTGGTTTAATTGTGACTTCTACAGGGATAT |
| | | TAATATTACTTCACTACTATGTGTTTAGTACAG |
| | | CGATTGGATTAACCTCCTTCGTCATTGCCATAT |
| | | TGGTTATTCAGGTGATAGCCTATATCCTCGCTG |
| | | TGGTTGGACTGAGTCTCTGTATTGCGGCGTGTA |
| | | TACCAATGCATGGCCCTCTTCTGATTTCAGGTT |
| | | TGAGTATCTTAGCTCTAGCACAATTACTTGGAC |
| | | TAGTTTATATGAAATTTGTGGCTTCCAATCAGA |
| | | AGACTATACAACCTCCTAGGAATAACTGA |
| 7 | CD47-202 | ATGTGGCCCCTGGTAGCGGCGCTGTTGCTGGGC |
| | (ENST00000361309.6) | TCGGCGTGCTGCGGATCAGCTCAGCTACTATTT |
| | cDNA | AATAAAACAAAATCTGTAGAATTCACGTTTTGT |
| | | AATGACACTGTCGTCATTCCATGCTTTGTTACT |
| | | AATATGGAGGCACAAAACACTACTGAAGTATAC |
| | | GTAAAGTGGAAATTTAAAGGAAGAGATATTTA |
| | | CACCTTTGATGGAGCTCTAAACAAGTCCACTGT |
| | | CCCCACTGACTTTAGTAGTGCAAAAATTGAAGT |
| | | CTCACAATTACTAAAAGGAGATGCCTCTTTGAA |
| | | GATGGATAAGAGTGATGCTGTCTCACACACAG |
| | | GAAACTACACTTGTGAAGTAACAGAATTAACC |
| | | AGAGAAGGTGAAACGATCATCGAGCTAAAATA |
| | | TCGTGTTGTTTCATGGTTTTCTCCAAATGAAAAT |
| | | ATTCTTATTGTTATTTTCCCAATTTTTGCTATAC |
| | | TCCTGTTCTGGGGACAGTTTGGTATTAAAACAC |
| | | TTAAATATAGATCCGGTGGTATGGATGAGAAA |
| | | ACAATTGCTTTACTTGTTGCTGGACTAGTGATC |
| | | ACTGTCATTGTCATTGTTGGAGCCATTCTTTTCG |
| | | TCCCAGGTGAATATTCATTAAAGAATGCTACTG |
| | | GCCTTGGTTTAATTGTGACTTCTACAGGGATAT |
| | | TAATATTACTTCACTACTATGTGTTTAGTACAGC |
| | | GATTGGATTAACCTCCTTCGTCATTGCCATATTG |
| | | GTTATTCAGGTGATAGCCTATATCCTCGCTGTG |
| | | GTTGGACTGAGTCTCTGTATTGCGGCGTGTATA |
| | | CCAATGCATGGCCCTCTTCTGATTTCAGGTTTG |
| | | AGTATCTTAGCTCTAGCACAATTACTTGGACTA |
| | | GTTTATATGAAATTTGTGGCTTCCAATCAGAAG |
| | | ACTATACAACCTCCTAGGAAAGCTGTAGAGGA |
| | | ACCCCTTAATGCATTCAAAGAATCAAAAGGAAT |
| | | GATGAATGATGAATAA |
| 8 | CD47-203 | TGGACTGAGTCTCTGTATTGCGGCGTGTATACC |
| | (ENST00000398258.7) | AATGCATGGCCCTCTTCTGATTTCAGGTTTGAG |
| | cDNA | TATCTTAGCTCTAGCACAATTACTTGGACTAGT |
| | | TTATATGAAATTTGTGGGGTTCAGAGGGACACC |
| | | TACAGTCAGTTGAAAGGCAAGAAGAGACAAGC |
| | | TTCCAATCAGAAGACTATACAACCTCCTAGGAA |
| | | AGCTGTAGAGGAACCCCTTAATGCATTCAAAGA |
| | | ATCAAAAGGAATGATGAATGATGAATAA |
| 9 | CD47-204 | AGATATTTTCTTGTTCAATTTAAGGAGAGGTAA |
| | (ENST00000471694.1) | ATTTGGTATCAATAGAAAAAATGTTTCTGAAAA |
| | DNA | ATTTAAACCCTGGAAATGTATTTATGGCATGGA |
| | | GTCAGATGTTTCAGGGAGAGAAGAACAAATCAA |
| | | GAAGCATTGCAAGTATGCTCATATGGAATGCTT |
| | | AAGGCTTGTGGTTAAAAAATATATATATATGGC |
| | | TGTCAATGTCTTAGGCTCATGGTAGCAGCAGAA |
| | | ATCGTAATAATTCTTTTGTCACATGGGTTATAT |
| | | CCATATTGGAGAGAATTAACTCAGGTGAAATTA |
| | | ACTTGTACACTGTTTGGTTTTATAATATTTAGA |
| | | GGGATCACAACTGACTGATGTCCCTTTGAAGTA |
| | | CCATTCTTCATAAATCtttttttttCAGAATGG |
| | | GCCAGCCAACTGTGACATCCCTTGGATCGGAGA |
| | | TTTAGAACTAGAAAGTATTCTTTCTACATTATT |
| | | AGGGAAGAAAAGGAGTTACTTGGCGGTTAGCAA |
| | | TATTCTAttttgttttgttttgtttttagagac |
| | | agggtctcattatgttgaccaggctggcctcga |
| | | gctcctgggctcaagcaatgctcccacctcagc |
| | | ctcccaagtagctgggactacaggcatgtgcca |
| | | ctacacctggcAGTGTTTATTCTGATAAATACA |
| | | TTTATGAGCTCAAAAATGTAACTCTAAAACCTT |
| | | ATCTCTGAACTTCCATATTACCATCAGAAATTT |
| | | AGATAGTTGTTTAGTTCTCTTTTTCTTTGTAGA |
| | | ACATAGATATAAGGCATGGTTTCATTGAAGTCA |
| | | GTTGTATATACATGTAACTATCCTGATGTTCCC |
| | | AAATAAAGCTCTGTATTTCTGCTTAGTTTATTG |
| | | GGGAGGCTGCTAAATGTAGTGCATCCCAACCCA |
| | | TTTTACCCTGTTCTACTTTAAAAAGAGGTTGGC |
| | | TTCTTGTTTGGATACAAGGACCAAGTCACTCCC |
| | | CCAGGTTCCTCCACAGTAAGGGAGGCCTATTTA |
| | | AAGCCGCCCATGGCACTAACAGAAACTGGACTC |
| | | CTATGAGCTCAGATACATAACTGGGCCTCACAG |
| | | GGGTGGGACAGTATGTAGTCTAGGAATTGGAAG |
| | | GATCCATTCCATATCAAAGAACTGAAGCATCGT |
| | | GTTGCCCTCTCAGCAGCAAGAGTAAGGTGATGC |
| | | CCCTGTCAGTTATAGTTCCTGAGTTCCTCTGTC |
| | | TTTGATTCTTTGCCTATTAGCCAGCTAGCTCAC |
| | | CCTCTTGTTTATGCCACTGTTTTTTATCCTATT |
| | | CATGCCTTCTCACAGACAACTTTTCTTACCTAC |
| | | AGCTTTGGACTCATCCTTGTCTCctttctgttt |
| | | ctttttcactttcccttcccatcaccaactttc |
| | | tgggtttttttctgtttcttcTTAGAGTCCAGT |
| | | GGCAGGGAGAAACTTGTCAGTCCAGTCTGTTGC |
| | | CATTTTTCCTGTTTGAGAAAGACTCACCAGCTT |
| | | TTGGCTGGCTCACAGATTGGCTTTCCTTGGGTC |
| | | AGGACCCACCCTTTTCCCTGCCAGCTTTGGAAG |
| | | CTTGACAGAATTCGAGTGTGCAGTGGTGGTAAA |
| | | TAAATAGTAAGGAACACAGAGCAGTCCTGGAGG |
| | | CGTGCCTCCATCTGCTGATGAGAAAATCCAGTG |
| | | CTGTCATCCAGCCCAGGTCCCAGCGGAATGGGC |
| | | CTCTCTGTTCAGTAGGATCCCCCTCCTGCTGAG |
| | | TGGTTCATGGCATGTTTCTGTTCAACGCTTTTC |
| | | CATCTGTAGGATTCTTATTCTGTAtttatttgt |
| | | ttttttgggtttttttattttttgagatggagt |
| | | ctcgctctgtcgcccaggctggagtgcagtggc |
| | | acgaccccagctcgctgcagcctctgcctccca |
| | | ggacgagggagatcctcccacctcagccttcca |
| | | cgtagctgggactacaggcatgcaccacaggca |
| | | tgcaccaccacgccagctaatttttgtattttt |
| | | ggtagagacagggttgcatcatgttgcccaggc |
| | | tggtcttgaatgcctgagctcaagcaatctatt |
| | | tgccttggcctcccaaagtgctgggattacagg |
| | | catgagccaccacggccagccTTCTCATTTGtt |
| | | ttttttATAAGGAAGCTATCTCTTCTTCCCTCC |
| | | CCAACTAGGGTATTCTTTTTCCCTTTCGTCACT |
| | | TTGCTCATGTACTGTATTCCTTCAACTTCATTA |
| | | ATGAATCCATTTGGAAGCAGTGAAAAAGGCAAC |
| | | TCAGAAAGCTAAGAAGAAATAGATAGAGGAATA |
| | | CTCAGAGCTATCTGAGTATTTTCTTTAGTTTGT |
| | | TAGCTCTTTGGAGCTTTGAAACTGGAAAGACCC |
| | | AGGGAGTGATGTGGAGAAAGAGACTGAGCTTGT |
| | | AAGACACAGGAGCAGTGAGCTAAGGGAGATGGA |
| | | GTAGTGGGGACAAATTCTGGCACATTCTGTCTA |
| | | CACTCTGGGTAGATAGAGGAGGGAGGATGGAGC |
| | | ACCCATGGTGGGGGTATGTTGGTGACAGCATTT |
| | | TCCCACCAGCCAGTGTAACAAGTGGCTGATTTG |
| | | GGGGAAAGATGGCATAAACAAATGAGAGAATGT |
| | | GTTTACTATTTGATGTAGATGGGTTATTTGCTT |
| | | CATTTTTCAAATCAGTGTATATAATCAAGAATA |
| | | TTCAGCATGTTTGAATAGACTGTCAGAGCTGGA |
| | | ACTCTTTCATTAACATCTCTGGCACCTTTAGTT |
| | | TTAGCCCTGAACATTTTATCTTAAAATTAAACA |
| | | TTACCAAATGCCTTAGtttatttcatttattaa |
| | | atttatattcttatttgttatttatatCAGCTT |
| | | CCAATCAGAAGACTATACAACCTCCTAGGAATA |
| | | ACTGAAGTGAAGTGATGGACTCCGATTTGGAGA |
| | | GTAGTAAGACGTGAAAGGAATACACTTGTGTTT |
| | | AAGCACCATGGCCTTGATGATTCACTGTTGGGG |
| | | AGAAGAAACAAGAAAAGTAACTGGTTGTCACCT |
| | | ATGAGACCCTTACGTGATTGTTAGTTAAGTTTT |
| | | TATTCAAAGCAGCTGTAATTTAGTTAATAAAAT |
| | | AATTATGATCTATGTTGTTTGCCCAATTGAGAT |
| | | CCAGTTTTTTGTTGTTATTTTTAATCAATTAGG |
| | | GGCAATAGTAGAATGGACAATTTCCAAGAATGA |
| | | TGCCTTTCAGGTCCTAGGGCCTCTGGCCTCTAG |
| | | GTAACCAGTTTAAATTGGTTCAGGGTGATAACT |
| | | ACTTAGCACTGCCCTGGTGATTACCCAGAGATA |
| | | TCTATGAAAACCAGTGGCTTCCATCAAACCTTT |
| | | GCCAACTCAGGTTCACAGCAGCTTTGGGCAGTT |
| | | ATGGCAGTATGGCATTAGCTGAGAGGTGTCTGC |
| | | CACTTCTGGGTCAATGGAATAATAAATTAAGTA |
| | | CAGGCAGGAATTTGGTTGGGAGCATCTTGTATG |
| | | ATCTCCGTATGATGTGATATTGATGGAGATAGT |
| | | GGTCCTCATTCTTGGGGGTTGCCATTCCCACAT |
| | | TCCCCCTTCAACAAACAGTGTAACAGGTCCTTC |
| | | CCAGATTTAGGGTACTTTTATTGATGGATATGT |
| | | TTTCCTTTTATTCACATAACCCCTTGAAACCCT |
| | | GTCTTGTCCTCCTGTTACTTGCTTCTGCTGTAC |
| | | AAGATGTAGCACCTTTTCTCCTCTTTGAACATG |
| | | GTCTAGTGACACGGTAGCACCAGTTGCAGGAAG |
| | | GAGCCAGACTTGTTCTCAGAGCACTGTGTTCAC |
| | | ACTTTTCAGCAAAAATAGCTATGGTTGTAACAT |
| | | ATGTATTCCCTTCCTCTGATTTGAAGGCAAAAA |
| | | TCTACAGTGTTTCTTCACTTCTTTTCTGATCTG |
| | | GGGCATGAAAAAAGCAAGATTGAAATTTGAACT |
| | | ATGAGTCTCCTGCATGGCAACAAAATGTGTGTC |
| | | ACCATCAGGCCAACAGGCCAGCCCTTGAATGGG |
| | | GATTTATTACTGTTGTATCTATGTTGCATGATA |
| | | AACATTCATCACCTTCCTCCTGTAGTCCTGCCT |
| | | CGTACTCCCCTTCCCCTATGATTGAAAAGTAAA |
| | | CAAAACCCACATTTCCTATCCTGGTTAGAAGAA |
| | | AATTAATGTTCTGACAGTTGTGATCGCCTGGAG |
| | | TACTTTTAGACTTTTAGCATTCGTTTTTTACCT |
| | | GTTTGTGGATGTGTGTTTGTATGTGCATACGTA |
| | | TGAGATAGGCACATGCATCTTCTGTATGGACAA |
| | | AGGTGGGGTACCTACAGGAGAGCAAAGGTTAAT |
| | | TTTGTGCTTTTAGTAAAAACATTTAAATACAAA |
| | | GTTCTTTATTGGGTGGAATTATATTTGATGCAA |
| | | ATATTTGATCACTTAAAACTTTTAAAACTTCTA |
| | | GGTAATTTGCCACGCTTTTTGACTGCTCACCAA |
| | | TACCCTGTAAAAATACGTAATTCTTCCTGTTTG |
| | | TGTAATAAGATATTCATATTTGTAGTTGCATTA |
| | | ATAATAGTTATTTCTTAGTCCATCAGATGTTCC |
| | | CGTGTGCCTCTTTTATGCCAAATTGATTGTCAT |
| | | ATTTCATGTTGGGACCAAGTAGTTTGCCCATGG |
| | | CAAACCTAAATTTATGACCTGCTGAGGCCTCTC |
| | | AGAAAACTGAGCATACTAGCAAGACAGCTCTTC |
| | | TTGaaaaaaaaaatatgtatacacaaatatata |
| | | cgtatatctatatatacgtatgtatatacacac |
| | | atgtatattCTTCCTTGATTGTGTAGCTGTCCA |
| | | AAATAATAACATATATAGAGGGAGCTGTATTCC |
| | | TTTATACAAATCTGATGGCTCCTGCAGCACTTT |
| | | TTCCTTCTGAAAATATTTACATTTTGCTAACCT |
| | | AGTTTGTTACTTTAAAAATCAGTTTTGATGAAA |
| | | GGAGGGAAAAGCAGATGGACTTGAAAAAGATCC |
| | | AAGCTCCTATTAGAAAAGGTATGAAAATCTTTA |
| | | TAGTAAAATTTTTTATAAACTAAAGTTGTACCT |
| | | TTTAATATGTAGTAAACTCTCATTTATTTGGGG |
| | | TTCGCTCTTGGATCTCATCCATCCATTGTGTTC |
| | | TCTTTAATGCTGCCTGCCTTTTGAGGCATTCAC |
| | | TGCCCTAGACAATGCCACCAGAGATAGTGGGGG |
| | | AAATGCCAGATGAAACCAACTCTTGCTCTCACT |
| | | AGTTGTCAGCTTCTCTGGATAAGTGACCACAGA |
| | | AGCAGGAGTCCTCCTGCTTGGGCATCATTGGGC |
| | | CAGTTCCTTCTCTTTAAATCAGATTTGTAATGG |
| | | CTCCCAAATTCCATCACATCACATTTAAATTGC |
| | | AGACAGTGTTTTGCACATCATGTATCTGTTTTG |
| | | TCCCATAATATGCTTTTTACTCCCTGATCCCAG |
| | | TTTCTGCTGTTGACTCTTCCATTCAGTTTTATT |
| | | TATTGTGTGTTCTCACAGTGACACCATTTGTCC |
| | | TTTTCTGCAACAACCTTTCCAGCTACTTTTGCC |
| | | AAATTCTATTTGTCTTCTCCTTCAAAACATTCT |
| | | CCTTTGCAGTTCCTCTTCATCTGTGTAGCTGCT |
| | | CTTTTGTCTCTTAACTTACCATTCCTATAGTAC |
| | | TTTATGCATCTCTGCTTAGTTCTATTAGTTTTT |
| | | TGGCCTTGCTCTTCTCCTTGATTTTAAAATTCC |
| | | TTCTATAGCTAGAGCTTTTCTTTCTTTCATTCT |
| | | CTCTTCCTGCAGTGTTTTGCATACATCAGAAGC |
| | | TAGGTACATAAGTTAAATGATTGAGAGTTGGCT |
| | | GTATTTAGATTTATCACTTTTTAATAGGGTGAG |
| | | CTTGAGAGttttctttctttctgtttttttttt |
| | | ttgtttttttttttttttttttttttttttttt |
| | | ttGACTAATTTCACATGCTCTAAAAACCTTCAA |
| | | AGGTGATTATTTTTCTCCTGGAAACTCCAGGTC |
| | | CATTCTGTTTAAATCCCTAAGAATGTCAGAATT |
| | | AAAATAACAGGGCTATCCCGTAATTGGAAATAT |
| | | TTCTTTTTTCAGGATGCTATAGTCAATTTAGTA |
| | | AGTGACCACCAAATTGTTATTTGCACTAACAAA |
| | | GCTCAAAACACGATAAGTTTACTCCTCCATCTC |
| | | AGTAATAAAAATTAAGCTGTAATCAACCTTCTA |
| | | GGTTTCTCTTGTCTTAAAATGGGTATTCAAAAA |
| | | TGGGGATCTGTGGTGTATGTATGGAAACACATA |
| | | CTCCTTAATTTACCTGTTGTTGGAAACTGGAGA |
| | | AATGATTGTCGGGCAACCGtttattttttattg |
| | | tattttatttggttgagggatttttttatAAAC |
| | | AGTTTTACTTGTGTCATATTTTAAAATTACTAA |
| | | CTGCCATCACCTGCTGGGGTCCTTTGTTAGGTC |
| | | ATTTTCAGTGACTAATAGGGATAATCCAGGTAA |
| | | CTTTGAAGAGATGAGCAGTGAGTGACCAGGCAG |
| | | TTTTTCTGCCTTTAGCTTTGACAGTTCTTAATT |
| | | AAGATCATTGAAGACCAGCTTTCTCATAAATTT |
| | | CTCTTTTTGAAAAAAAGAAAGCATTTGTACTAA |
| | | GCTCCTCTGTAAGACAACATCTTAAATCTTAAA |
| | | AGTGTTGTTATCATGACTGGTGAGAGAAGAAAA |
| | | CATTTTGTTTTTATTAAATGGAGCATTATTTAC |
| | | AAAAAGCCATTGTTGAGAATTAGATCCCACATC |
| | | GTATAAATATCTATTAACCATTCTAAATAAAGA |
| | | GAACTCCAGTGTTGCTATGTGCAAGATCCTCTC |
| | | TTGGAGCTTTTTTGCATAGCAATTAAAGGTGTG |
| | | CTATTTGTCAGTAGCCATTTTTTTGCAGTGATT |
| | | TGAAGACCAAAGTTGTTTTACAGCTGTGTTACC |
| | | GTTAAAGGtttttttttttATATGTATTAAATC |
| | | AATTTATCACTGTTTAAAGCTTTGAATATCTGC |
| | | AATCTTTGCCAAGGTACTTTTTTATTTaaaaaa |
| | | aaaCATAACTTTGTAAATATTACCCTGTAATAT |
| | | TATATATACTTAATAAAACATTTTAAGCTAT |
| 10 | CD47-205 | AAGAATGCTACTGGCCTTGGTTTAATTGTGACT |
| | (ENST00000517766.5) | TCTACAGGGATATTAATATTACTTCACTACTAT |
| | cDNA | GTGTTTAGTACAGCGATTGGATTAACCTCCTTC |
| | | GTCATTGCCATATTGGTTATTCAGGTGATAGCC |
| | | TATATCCTCGCTGTGGTTGGACTGAGTCTCTGT |
| | | ATTGCGGCGTGTATACCAATGCATGGCCCTCTT |
| | | CTGATTTCAGGTTTGAGTATCTTAGCTCTAGCA |
| | | CAATTACTTGGACTAGTTTATATGAAATTTGTG |
| | | GCTTCCAATCAGAAGACTATACAACCTCCTAGG |
| | | AAAGCTGTAGAGGAACCCCTTAATGAATAA |
| 11 | CD47-206 | ATGGAGGCACAAAACACTACTGAAGTATACGT |
| | (ENST00000644850.1) | AAAGTGGAAATTTAAAGGAAGAGATATTTACA |
| | cDNA | CCTTTGATGGAGCTCTAAACAAGTCCACTGTCC |
| | | CCACTGACTTTAGTAGTGCAAAAATTGAAGTCT |
| | | CACAATTACTAAAAGGAGATGCCTCTTTGAAGA |
| | | TGGATAAGAGTGATGCTGTCTCACACACAGGA |
| | | AACTACACTTGTGAAGTAACAGAATTAACCAG |
| | | AGAAGGTGAAACGATCATCGAGCTAAAATATC |
| | | GTGTTGTTTCATGGTTTTCTCCAAATGAAAATAT |
| | | TCTTATTGTTATTTTCCCAATTTTTGCTATACTC |
| | | CTGTTCTGGGGACAGTTTGGTATTAAAACACTT |
| | | AAATATAGATCCGGTGGTATGGATGAGAAAAC |
| | | AATTGCTTTACTTGTTGCTGGACTAGTGATCACT |
| | | GTCATTGTCATTGTTGGAGCCATTCTTTTCGTCC |
| | | CAGGTGAATATTCATTAAAGAATGCTACTGGCC |
| | | TTGGTTTAATTGTGACTTCTACAGGGATATTAA |
| | | TATTACTTCACTACTATGTGTTT |

In some embodiments, all or a functional portion of CD47 can be linked to other components such as a signal peptide, a leader sequence, a secretory signal, a label (e.g., a reporter gene), or any combination thereof. In some embodiments, the nucleic acid sequence encoding a signal peptide of CD47 is replaced with a nucleic acid sequence encoding a signal peptide from a heterologous protein. The heterologous protein can be, for example, CD8α, CD28, tissue plasminogen activator (tPA), growth hormone, granulocyte-macrophage colony stimulating factor (GM-CSF), GM-CSF receptor (GM-CSFRa), or an immunoglobulin (e.g., IgE or IgK). In some embodiments, the signal peptide is a signal peptide from an immunoglobulin (such as IgG heavy chain or IgG-kappa light chain), a cytokine (such as interleukin-2 (IL-2), or CD33), a serum albumin protein (e.g., HSA or albumin), a human azurocidin preprotein signal sequence, a luciferase, a trypsinogen (e.g., chymotrypsinogen or trypsinogen) or other signal peptide able to efficiently express a protein by or on a cell.

In some embodiments, the engineered cell or population of cells (e.g., T cells, B cells, NK cells, islet cells such as beta cells, or hypoimmunogenic cells thereof) contains a transgene that encodes CD200, such as human CD200, or a functional fragment or variant thereof. In some embodiments, CD200 is overexpressed in the cell. In some embodiments, the expression of CD200 is increased in the engineered cell or population of cells compared to a similar reference or non-engineered cell (including with any other modifications) except that the reference or non-engineered cell does not include the transgene encoding CD200. Useful genomic, polynucleotide and polypeptide information about human CD200 are provided in, for example, the GeneCard Identifier GC03P112332, HGNC No. 7203, NCBI Gene ID 4345, Uniprot No. P41217, and NCBI RefSeq Nos. NP_001004196.2, NM_001004196.3, NP_001305757.1, NM_001318828.1, NP_005935.4, NM_005944.6, XP_005247539.1, and XM_005247482.2. In certain embodiments, the transgene encoding CD200, or a functional fragment or variant thereof, is operably linked to a promoter.

In some embodiments, the engineered cell or population of cells (e.g., T cells, B cells, NK cells, islet cells such as beta cells, or hypoimmunogenic cells thereof) contains a transgene that encodes HLA-E, such as human HLA-E, or a functional fragment or variant thereof. In some embodiments, HLA-E is overexpressed in the cell. In some embodiments, the expression of HLA-E is increased in the engineered cell or population of cells compared to a similar reference or non-engineered cell (including with any other modifications) except that the reference or non-engineered cell does not include the transgene encoding HLA-E. Useful genomic, polynucleotide, and polypeptide information about human HLA-E are provided in, for example, the GeneCard Identifier GC06P047281, HGNC No. 4962, NCBI Gene ID 3133, Uniprot No. P13747, and NCBI RefSeq Nos. NP_005507.3 and NM_005516.5. In certain embodiments, the transgene encoding HLA-E, or a functional fragment or variant thereof, is operably linked to a promoter.

In some embodiments, the engineered cell or population of cells (e.g., T cells, B cells, NK cells, islet cells such as beta cells, or hypoimmunogenic cells thereof) contains a transgene that encodes HLA-G, such as human HLA-G, or a functional fragment or variant thereof. In some embodiments, HLA-G is overexpressed in the cell. In some embodiments, the expression of HLA-G is increased in the engineered cell or population of cells compared to a similar reference or non-engineered cell (including with any other modifications) except that the reference or non-engineered cell does not include the transgene encoding HLA-G. Useful genomic, polynucleotide and polypeptide information about human HLA-G are provided in, for example, the GeneCard Identifier GC06P047256, HGNC No. 4964, NCBI Gene ID 3135, Uniprot No. P17693, and NCBI RefSeq Nos. NP_002118.1 and NM_002127.5. In certain embodiments, the transgene encoding HLA-G, or a functional fragment or variant thereof, is operably linked to a promoter.

In some embodiments, the engineered cell or population of cells (e.g., T cells, B cells, NK cells, islet cells such as beta cells, or hypoimmunogenic cells thereof) contains a transgene that encodes PD-L1, such as human PD-L1, or a functional fragment or variant thereof. In some embodiments, PD-L1 is overexpressed in the cell. In some embodiments, the expression of PD-L1 is increased in the engineered cell or population of cells compared to a similar reference or non-engineered cell (including with any other modifications) except that the reference or non-engineered cell does not include the transgene encoding PD-L1. Useful genomic, polynucleotide and polypeptide information about human PD-L1 or CD274 are provided in, for example, the GeneCard Identifier GC09P005450, HGNC No. 17635, NCBI Gene ID 29126, Uniprot No. Q9NZQ7, and NCBI RefSeq Nos. NP_001254635.1, NM_001267706.1, NP_054862.1, and NM_014143.3. In certain embodiments, the polynucleotide encoding PD-L1, or a functional fragment or variant thereof, is operably linked to a promoter.

In some embodiments, the engineered cell or population of cells (e.g., T cells, B cells, NK cells, islet cells such as beta cells, or hypoimmunogenic cells thereof) contains a transgene that encodes FasL, such as human FasL, or a functional fragment or variant thereof. In some embodiments, FasL is overexpressed in the cell. In some embodiments, the expression of FasL is increased in the engineered cell or population of cells compared to a similar reference or non-engineered cell (including with any other modifications) except that the reference or non-engineered cell does not include the transgene encoding FasL. Useful genomic, polynucleotide and polypeptide information about human Fas ligand (which is known as FasL, FASLG, CD178, TNFSF6, and the like) are provided in, for example, the GeneCard Identifier GC01P172628, HGNC No. 11936, NCBI Gene ID 356, Uniprot No. P48023, and NCBI RefSeq Nos. NP_000630.1, NM_000639.2, NP_001289675.1, and NM_001302746.1. In certain embodiments, the transgene encoding Fas-L is operably linked to a promoter.

In some embodiments, the engineered cell or population of cells (e.g., T cells, B cells, NK cells, islet cells such as beta cells, or hypoimmunogenic cells thereof) contains a transgene that encodes CCL21, such as human CCL21, or a functional fragment or variant thereof. In some embodiments, CCL21 is overexpressed in the cell. In some embodiments, the expression of CCL21 is increased in the engineered cell or population of cells compared to a similar reference or non-engineered cell (including with any other modifications) except that the reference or non-engineered cell does not include the transgene encoding CCL21. Useful genomic, polynucleotide and polypeptide information about human CCL21 are provided in, for example, the GeneCard Identifier GC09M034709, HGNC No. 10620, NCBI Gene ID 6366, Uniprot No. 000585, and NCBI RefSeq Nos. NP_002980.1 and NM_002989.3. In certain embodiments, the transgene encoding CCL21, or a functional fragment or variant thereof, is operably linked to a promoter.

In some embodiments, the engineered cell or population of cells (e.g., T cells, B cells, NK cells, islet cells such as beta cells, or hypoimmunogenic cells thereof) contains a transgene that encodes CCL22, such as human CCL22, or a functional fragment or variant thereof. In some embodiments, CCL22 is overexpressed in the cell. In some embodiments, the expression of CCL22 is increased in the engineered cell or population of cells compared to a similar reference or non-engineered cell (including with any other modifications) except that the reference or non-engineered cell does not include the transgene encoding CCL22. Useful genomic, polynucleotide and polypeptide information about human CCL22 are provided in, for example, the GeneCard Identifier GC16P057359, HGNC No. 10621, NCBI Gene ID 6367, Uniprot No. 000626, and NCBI RefSeq Nos. NP_002981.2, NM_002990.4, XP_016879020.1, and XM_017023531.1. In certain embodiments, the transgene encoding CCL22, or a functional fragment or variant thereof, is operably linked to a promoter.

In some embodiments, the engineered cell or population of cells (e.g., T cells, B cells, NK cells, islet cells such as beta cells, or hypoimmunogenic cells thereof) contains a transgene that encodes Mfge8, such as human Mfge8, or a functional fragment or variant thereof. In some embodiments, Mfge8 is overexpressed in the cell. In some embodiments, the expression of Mfge8 is increased in the engineered cell or population of cells compared to a similar reference or non-engineered cell or population of cells (including with any other modifications) except that the reference or non-engineered cell or population of cells does not include the transgene encoding Mfge8. Useful genomic, polynucleotide and polypeptide information about human Mfge8 are provided in, for example, the GeneCard Identifier GC15M088898, HGNC No. 7036, NCBI Gene ID 4240, Uniprot No. Q08431, and NCBI RefSeq Nos. NP_001108086.1, NM_001114614.2, NP_001297248.1, NM_001310319.1, NP_001297249.1, NM_001310320.1, NP_001297250.1, NM_001310321.1, NP_005919.2, and NM_005928.3. In certain embodiments, the transgene encoding Mfge8, or a functional fragment or variant thereof, is operably linked to a promoter.

In some embodiments, the engineered cell or population of cells (e.g., T cells, B cells, NK cells, islet cells such as beta cells, or hypoimmunogenic cells thereof) contains a transgene that encodes SerpinB9, such as human SerpinB9, or a functional fragment or variant thereof. In some embodiments, SerpinB9 is overexpressed in the cell. In some embodiments, the expression of SerpinB9 is increased in the engineered cell or population of cells compared to a similar reference or non-engineered cell or population of cells (including with any other modifications) except that the reference or non-engineered cell or population of cells does not include the transgene encoding SerpinB9. Useful genomic, polynucleotide and polypeptide information about human SerpinB9 are provided in, for example, the GeneCard Identifier GC06M002887, HGNC No. 8955, NCBI Gene ID 5272, Uniprot No. P50453, and NCBI RefSeq Nos. NP_004146.1, NM_004155.5, XP_005249241.1, and XM_005249184.4. In certain embodiments, the transgene encoding SerpinB9, or a functional fragment or variant thereof, is operably linked to a promoter.

### (2) Insertion of Exogenous Sequences at Endogenous Proliferation Genes or Off-Target Cell Marker Genes

In some embodiments, provided herein is a modified cell comprising one or more exogenous sequences inserted at endogenous proliferation genes or off-target cell marker genes, such as any of endogenous proliferation genes or off-target cell marker genes described above or known in the art. Exogenous sequences, such as any of the exogenous sequences described below, may be inserted at endogenous proliferation genes or off-target cell marker genes by various methods and agents, for example, using zinc finger nucleases, restriction enzymes, transcription activator-like effector nucleases (TALENs), Programmable Addition via Site-specific Targeting Elements (PASTE), nucleic acid-guided nuclease editing, and the like. In some embodiments, exogenous sequences, such as any of the exogenous sequences described below, may be inserted at endogenous proliferation genes or off-target cell marker genes using a genome editing complex, as described further below.

a. Genome Editing Complexes

In some embodiments, one or more exogenous sequences, such as any of the exogenous sequences described below, may be inserted at endogenous proliferation genes or off-target cell marker genes using a genome editing complex.

Any of a variety of agents associated with gene editing technologies can be included in a genome editing complex. The genome editing complex can be used for knock-in or integration of DNA sequences into a region of the genome. In some embodiments, the genome editing complex mediates single-strand breaks (SSB). In some embodiments, the genome editing complex mediates double-strand breaks (DSB), including in connection with non-homologous end-joining (NHEJ) or homology-directed repair (HDR). In some embodiments, the genome editing complex does not mediate SSB. In some embodiments, the genome editing complex does not mediate DSB. In some embodiments, the genome editing complex can be used for DNA base editing or prime-editing. In some embodiments, the genome editing complex can be used for Programmable Addition via Site-specific Targeting Elements (PASTE). In some embodiments, the genome editing complex can cleave, deaminate, nick, polymerize, interrogate, integrate, cut, unwind, break, alter, methylate, demethylate, or otherwise destabilize a target locus.

In some embodiments, the one or more exogenous sequences are inserted at endogenous proliferation genes or off-target cell marker genes by one or more gene edits. In some embodiments, said one or more gene edits are made by a genome editing complex. In some embodiments, a cell of the disclosure comprises a genome editing complex, e.g., which can insert one or more exogenous sequences at endogenous proliferation genes or off-target cell marker genes.

In some embodiments, the genome editing complex is or encodes one or more polypeptides having an activity selected from nuclease activity (e.g., programmable nuclease activity); nickase activity (e.g., programmable nickase activity); homing activity (e.g., programmable DNA binding activity); nucleic acid polymerase activity (e.g., DNA polymerase or RNA polymerase activity); integrase activity; recombinase activity; or base editing activity (e.g., cytidine deaminase or adenosine deaminase activity).

In some embodiments, the genome editing complex is one for use in target-primed reverse transcription (TPRT) or “prime editing”. In some embodiments, prime editing mediates targeted insertions, deletions, all 12 possible base-to-base conversions, and combinations thereof in human cells without requiring DSBs or donor DNA templates. Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors search and locate the desired target site to be edited, and encode a replacement strand containing a desired edit which is installed in place of the corresponding target site endogenous DNA strand at the same time. For example, prime editing can be adapted for conducting precision CRISPR/Cas-based genome editing in order to bypass double stranded breaks. In some embodiments, the genome editing complex is or encodes for a primer editor that is a reverse transcriptase, or any DNA polymerase known in the art. Thus, in one aspect, the prime editor may comprise Cas9 (or an equivalent napDNAbp) which is programmed to target a DNA sequence by associating it with a specialized guide RNA (i.e., PEgRNA) containing a spacer sequence that anneals to a complementary protospacer in the target DNA. Such methods include any disclosed in Anzalone et al., (doi.org/10.1038/s41586-019-1711-4), or in PCT publication Nos. WO2020191248, WO2021226558, or WO2022067130, which are hereby incorporated in their entirety. In some embodiments, the genome editing complex is or encodes for a Cas protein-reverse transcriptase fusion or related systems to target a specific DNA sequence with a guide RNA, generate a single strand nick at the target site, and use the nicked DNA as a primer for reverse transcription of an engineered reverse transcriptase template that is integrated with the guide RNA. In some embodiments, the prime editor protein is paired with two prime editing guide RNAs (pegRNAs) that template the synthesis of complementary DNA flaps on opposing strands of genomic DNA, resulting in the replacement of endogenous DNA sequence between the PE-induced nick sites with pegRNA-encoded sequences.

In some embodiments, the genome editing complex is or encodes a base editor (e.g., a nucleobase editor). Base editors (BEs) are typically fusions of a Cas (“CRISPR-associated”) domain and a nucleobase modification domain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”) domains. In some cases, base editors may also include proteins or domains that alter cellular DNA repair processes to increase the efficiency and/or stability of the resulting single-nucleotide change. Currently available base editors include cytidine base editors (e.g., BE4) that convert target C·G to T·A and adenine base editors (e.g., ABE7.10) that convert target A·T to G·C. Cas9-targeted deamination was first demonstrated in connection with a Base Editor (BE) system designed to induce base changes without introducing double-strand DNA breaks. Further Rat deaminase APOBEC1 (rAPOBEC1) fused to deactivated Cas9 (dCas9) was used to successfully convert cytidines to thymidines upstream of the PAM of the sgRNA. This first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U:A base pair, which is then converted to T:A during DNA replication. In some embodiments, the genome editing complex is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, the base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, the base editor is a fusion protein that comprises a DNA domain of a CRISPR-Cas (e.g., Cas9) having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, the base editor is an adenine-to-thymine or “ATBE” (or thymine-to-adenine or “TABE”) transversion base editor. Exemplary base editor and base editor systems include any as described in patent publication Nos. US20220127622, US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, the genome editing complex is for use in Programmable Addition via Site-specific Targeting Elements (PASTE). In some aspects, PASTE is a platform in which genomic insertion is directed via a CRISPR-Cas9 nickase fused to both a reverse transcriptase and serine integrase. As described in Ioannidi et al. (doi.org/10.1101/2021.11.01.466786), PASTE does not generate double stranded breaks but allows for integration of sequences as large as ˜36 kb. In some embodiments, the serine integrase can be any known in the art. In some embodiments, the serine integrase has sufficient orthogonality such that PASTE can be used for multiplexed gene integration, simultaneously integrating at least two different genes in at least two genomic loci. In some embodiments, PASTE has editing efficiencies comparable to or better than those of homology directed repair or non-homologous end joining based integration, with activity in nondividing cells and fewer detectable off-target events.

In some embodiments, the genome editing complex comprises a genome targeting entity and/or a genome modifying entity.

In some embodiments, the genome targeting entity is a nucleic acid-guided targeting entity, such as any of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising a gRNA and a Cas nuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof. In other embodiments, the genome targeting entity comprises any of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.

In some embodiments, the genome modifying entity comprises one or more genome modifying activities, such as any of: cleaving, deaminating, nicking, polymerizing, interrogating, integrating, cutting, unwinding, breaking, altering, methylating, demethylating, or otherwise destabilizing a target locus, as well as any combination thereof. In some embodiments, the genome modifying entity is selected from Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor, a target-primed reverse transcription (TPRT) editor, APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, a transcriptional repressor, or a functional portion thereof. In other embodiments, the genome modifying entity is selected from a sequence specific nuclease, a recombinant nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof. In some specific embodiments, the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof. In some embodiments, the genome modifying entity is a Cas protein, a transcription activator-like effector nuclease (TALEN), or a zinc finger nuclease (ZFN). In some embodiments, the recombinant nuclease is a Cas nuclease. In some embodiments, the recombinant nuclease is a TALEN. In some embodiments, the recombinant nuclease is a ZFN.

In some embodiments, the genome targeting entity and the genome modifying entity in a genome editing complex are different domains of a single polypeptide. In other embodiments, the genome targeting entity and genome modifying entity are two different polypeptides that are operably linked together. In certain embodiments, the genome targeting entity and genome modifying entity are two different polypeptides that are not linked together.

In some specific embodiments, the genome editing complex comprises a guide nucleic acid having a targeting sequence that is complementary to at least one target locus, optionally wherein the guide nucleic acid is a guide RNA (gRNA). In some embodiments, the genome editing complex is an RNA-guided nuclease, such as a Cas nuclease (e.g., a Type II or Type V Cas protein) and a guide RNA (CRISPR-Cas combination). In some embodiments, the CRISPR-Cas combination is a ribonucleoprotein (RNP) complex comprising the gRNA and the Cas nuclease. Exemplary and non-limiting Cas nucleases that may be used in the genome editing complexes of the disclosure include Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. In some embodiments, the Cas protein is selected from Cas3, Cas9, Cas10, Cas12, Cas13, or Mad7. In some embodiments, the Cas protein is Mad7. In some embodiments, the Cas protein is a Cas12a (also known as cpf1) from a Prevotella, Francisella novicida, Acidaminococcus sp., Lachnospiraceae bacterium, or Francisella bacteria. In some embodiments, the Cas protein is a Cas12b from a Bacillus, optionally Bacillus hisashii. In some embodiments, the Cas protein is Cas9 from Streptococcus pyogenes (SpCas). In some embodiments, the Cas9 protein is from Staphylococcus aureus (SaCas9). In some embodiments, the Cas9 protein is from Neisseria meningitidis (NmeCas9). In some embodiments, the Cas9 protein is from Campylobacter jejuni (CjCas9). In some embodiments, the Cas9 protein is from Streptococcus thermophilis (StCas9). The Cas9 nuclease can, in some embodiments, be a Cas9 or functional fragment thereof from any bacterial species. See, e.g., Makarova et al. Nature Reviews, Microbiology, 9: 467-477 (2011), including supplemental information, hereby incorporated by reference in its entirety.

In some embodiments, the Cas is wild-type Cas9, which can site-specifically cleave double-stranded DNA, resulting in the activation of the double-strand break (DSB) repair machinery. DSBs can be repaired by the cellular Non-Homologous End Joining (NHEJ) pathway (Overballe-Petersen et al., 2013, Proc Natl Acad Sci USA, Vol. 110: 19860-19865), resulting in insertions and/or deletions (indels), which disrupt the targeted locus. Alternatively, if a donor template with homology to the targeted locus is supplied, the DSB may be repaired by the homology-directed repair (HDR) pathway allowing for precise replacement mutations to be made (Overballe-Petersen et al., 2013, Proc Natl Acad Sci USA, Vol. 110: 19860-19865; Gong et al., 2005, Nat. Struct Mol Biol, Vol. 12: 304-312). In some embodiments, the Cas is a mutant form known as Cas9 D10A, with only nickase activity. This means that Cas9D10A cleaves only one DNA strand and does not activate NHEJ. Instead, when provided with a homologous repair template, DNA repairs are conducted via the high-fidelity HDR pathway only, resulting in reduced indel mutations (Cong et al., 2013, Science, Vol. 339: 819-823; Jinek et al., 2012, Science, Vol. 337: 816-821; Qi et al., 2013 Cell, Vol. 152: 1173-1183). Cas9D10A is even more appealing in terms of target specificity when loci are targeted by paired Cas9 complexes designed to generate adjacent DNA nicks (Ran et al., 2013, Cell, Vol. 154: 1380-1389). In some embodiments, the Cas is a nuclease-deficient Cas9 (Qi et al., 2013 Cell, Vol. 152: 1173-1183). For instance, mutations H840A in the HNH domain and D10A in the RuvC domain inactivate cleavage activity, but do not prevent DNA binding. Therefore, this variant can be used to target in a sequence-specific manner any region of the genome without cleavage. Instead, by fusing with various effector domains, dCas9 can be used either as a gene silencing or activation tool. Furthermore, it can be used as a visualization tool by coupling the guide RNA or the Cas9 protein to a fluorophore or a fluorescent protein.

In some embodiments, the Cas protein comprises one or more mutations such that the Cas protein is converted into a nickase that is able to cleave only one strand of a double stranded DNA molecule (e.g., an SSB). For example, Cas9, which is normally capable of inducing a double strand break, can be converted into a Cas9 nickase, which is capable of inducing a single strand break, by mutating one of two Cas9 catalytic domains: the RuvC domain, which comprises the RuvC I, RuvC II, and RuvC III motifs, or the NHN domain. In some embodiments, the Cas protein comprises one or more mutations in the RuvC catalytic domain or the HNH catalytic domain. In some embodiments, the genome modifying entity is a recombinant nuclease that has been modified to have nickase activity. In some embodiments, the recombinant nuclease cleaves the strand to which the guide RNA, e.g., sgRNA, hybridizes, but does not cleave the strand that is complementary to the strand to which the guide RNA, e.g., sgRNA, hybridizes. In some embodiments, the recombinant nuclease does not cleave the strand to which the guide RNA, e.g., sgRNA, hybridizes, but does cleave the strand that is complementary to the strand to which the guide RNA, e.g., sgRNA, hybridizes.

In some embodiments, the genome editing complex is capable of inducing a DSB and comprises a nuclease or a functional fragment thereof, and a first guide RNA, e.g., a first sgRNA, and a second guide RNA, e.g., a second sgRNA. The guide RNA, e.g., the first guide RNA or the second guide RNA, in some embodiments, binds to the nuclease and targets the nuclease to a specific location within the target gene such as at a location within the sense strand or the antisense strand of the target gene that is or includes the cleavage site. In some embodiments, the recombinant nuclease is a Cas protein from any bacterial species or is a functional fragment thereof. In some embodiments, the Cas protein is Cas9 nuclease. Cas9 can, in some embodiments, be a Cas9 or functional fragment thereof from any bacterial species. See, e.g., Makarova et al., Nature Reviews, Microbiology, 9: 467-477 (2011), including supplemental information, hereby incorporated by reference in its entirety.

In some embodiments, the Cas9 is from Streptococcus pyogenes (SpCas9) and comprises one or more mutations in the RuvC catalytic domain or the HNH catalytic domain. In some embodiments, the one or more mutations in the RuvC catalytic domain or the HNH catalytic domain inactivates the catalytic activity of the domain. In some embodiments, the recombinant nuclease has RuvC activity but does not have HNH activity. In some embodiments, the recombinant nuclease does not have RuvC activity but does have HNH activity. In some embodiments, the Cas9 is from Streptococcus pyogenes (SpCas9) and comprises one or more mutations selected from the group consisting of D10A, H840A, H854A, and H863A. In some embodiments, the Cas9 is from Streptococcus pyogenes (SpCas9) and comprises one or more mutations in the RuvC I, RuvC II, or RuvC III motifs. In some embodiments, the Cas9 is from Streptococcus pyogenes (SpCas9) and comprises a mutation in the RuvC I motif. In some embodiments, the Cas9 is from Streptococcus pyogenes (SpCas9) and comprises a D10A mutation in the RuvC I motif. In some embodiments, the Cas9 is from Streptococcus pyogenes (SpCas9) and comprises one or more mutations in the HNH catalytic domain. In some embodiments, the one or more mutations in the HNH catalytic domain is selected from the group consisting of H840A, H854A, and H863A. In some embodiments, the Cas9 is from Streptococcus pyogenes (SpCas9) and comprises a H840A mutation in the HNH catalytic domain. In some embodiments, the Cas9 is from Streptococcus pyogenes (SpCas9) and comprises a H840A mutation. In some embodiments, the Cas9 is from Streptococcus pyogenes (SpCas9) and comprises a D10A mutation. In some embodiments, the Cas9 is from Streptococcus pyogenes (SpCas9) and comprises one or more mutations selected from the group consisting of N497A, R661A, Q695A, and Q926A. In some embodiments, the Cas9 is from Streptococcus pyogenes (SpCas9) and comprises one or more mutations selected from the group consisting of R780A, K810A, K855A, H982A, K1003A, R1060A, and K848A. In some embodiments, the Cas9 is from Streptococcus pyogenes (SpCas9) and comprises one or more mutations selected from the group consisting of N692A, M694A, Q695A, and H698A. In some embodiments, the Cas9 is from Streptococcus pyogenes (SpCas9) and comprises one or more mutations selected from the group consisting of M495V, Y515N, K526E, and R661Q. In some embodiments, the Cas9 is from Streptococcus pyogenes (SpCas9) and comprises one or more mutations selected from the group consisting of F539S, M763I, and K890N. In some embodiments, the Cas9 is from Streptococcus pyogenes (SpCas9) and comprises one or more mutations selected from the group consisting of E480K, E543D, E1219V, A262T, S409I, M694I, E108G, S217A.

In some embodiments, the Cas9 is from Streptococcus pyogenes (SaCas9). In some embodiments, the SaCas9 is wild-type SaCas9. In some embodiments, the SaCas9 comprises one or more mutations in REC3 domain. In some embodiments, the SaCas9 comprises one or more mutations in REC1 domain. In some embodiments, the SaCas9 comprises one or more mutations selected from the group consisting of N260D, N260Q, N260E, Q414A, Q414L. In some embodiments, the SaCas9 comprises one or more mutations in the recognition lobe. In some embodiments, the SaCas9 comprises one or more mutations selected from the group consisting of R245A, N413A, N419A. In some embodiments, the SaCas9 comprises one or more mutations in the RuvC-III domain. In some embodiments, the SaCas9 comprises a R654A mutation.

In some embodiments, the Cas protein is Cas12. In some embodiments, the Cas protein is Cas12a (i.e., cpf1). In some embodiments, the Cas12a is from the group consisting of Francisella novicida U112 (FnCas12a), Acidaminococcus sp. BV3L6 (AsCas12a), Moraxella bovoculi AAX11_00205 (Mb3Cas12a), Lachnospiraceae bacterium ND2006 (LbCas12a), Thiomicrospira sp. Xs5 (TsCas12a), Moraxella bovoculi AAX08_00205 (Mb2Cas12a), and Butyrivibrio sp. NC3005 (BsCas12a). In some embodiments, the Cas12a recognizes a T-rich 5′ protospacer adjacent motif (PAM). In some embodiments, the Cas12a processes its own crRNA without requiring a transactivating crRNA (tracrRNA). In some embodiments, the Cas12a processes both RNase and DNase activity. In some embodiments, the Cas12a is a split Cas12a platform, consisting of N-terminal and C-terminal fragments of Cas12a. In some embodiments, the split Cas12a platform is from Lachnospiraceae bacterium.

In some embodiments, the Cas protein is a Mad7 protein. Mad7 is an engineered class 2 type V-A CRISPR-Cas (Cas12a/Cpf1) system isolated from Eubacterium rectale. Mad7 is an engineered Cas12a variant with 76% homology to wild-type Cas12a. Mad7 is highly proficient in generating genomic insertions and/or deletions (indels) indels, small DNA insertions (e.g., 23 bases), and larger integrations ranging from about 1 to 14 kb in size (see, e.g., Liu, Z. et al., CRISPR J, 3(2):97-108, 2020). Mad7 only requires a crRNA for gene editing and allows for specific targeting of AT rich regions of the genome.

In some embodiments, the Cas is a TnpB protein. In some embodiments, TnpB proteins may comprise a Ruv-C-like domain. The RuvC domain may be a split RuvC domain comprising RuvC-I, RuvC-II, and RuvC-III subdomains. In some embodiments, the TnpB may further comprise one or more of a HTH domain, a bridge helix domain, and a zinc finger domain. TnpB proteins do not comprise an HNH domain. In some embodiments, a TnpB protein comprises, starting at the N-terminus: a HTH domain, a RuvC-I subdomain, a bridge helix domain, a RuvC-II sub-domain, a zinger finger domain, and a RuvC-III sub-domain. In some embodiments, a RuvC-III sub-domain forms the C-terminus of a TnpB protein. In some embodiments, the TnpB protein is from Epsilonproteobacteria bacterium, Actinoplanes lobatus strain DSM 43150, Actinomadura celluolosilytica strain DSM 45823, Actinomadura namibiensis strain DSM 44197, Alicyclobacillus macrosprangiidus strain DSM 17980, Lipingzhangella halophila strain DSM 102030, or Ktedonobacter recemifer. In some embodiments, the TnpB protein is from Ktedonobacter racemifer, or comprises a conserved RNA region with similarity to the 5′ ITR of K. racemifer TnpB loci. In some embodiments, the TnpB may comprise a Fanzor protein, a TnpB homolog found in eukaryotic genomes.

In some embodiments, the genome editing complex comprises, or is used in combination with, a guide RNA, e.g., single guide RNA (sgRNA), for inducing a DSB at the cleavage site. In some embodiments, the genome editing complex comprises, or is used in combination with, more than one guide RNA, e.g., a first sgRNA and a second sgRNA, for inducing a DSB at the cleavage site through an SSB on each strand. In some embodiments, the genome editing complex can be used in combination with a donor template, e.g., a single-stranded DNA oligonucleotide (ssODN), for HDR-mediated integration of the donor template into the target gene, such as at the targeting sequence. In some embodiments, the genome editing complex can be used in combination with a donor template, e.g., an ssODN, and a guide RNA, e.g., a sgRNA, for HDR-mediated integration of the donor template into the target gene, such as at the targeting sequence. In some embodiments, the genome editing complex can be used in combination with a donor template, e.g., an ssODN, and a first guide RNA, e.g., a first sgRNA, and a second guide RNA, e.g., a second sgRNA, for HDR-mediated integration of the donor template into the target gene, such as at the targeting sequence.

In particular embodiments, the genome-modifying agent is a Cas protein, such as Cas9. In some embodiments, delivery of the CRISPR/Cas can be used to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations.

In some embodiments, the genome-modifying agent is targeted to the cleavage site by interacting with a guide RNA, e.g., sgRNA, that hybridizes to a DNA sequence that immediately precedes a Protospacer Adjacent Motif (PAM) sequence or a target adjacent motif or transposon-associated motif (TAM) sequence. In general, a guide RNA, e.g., sgRNA, is any nucleotide sequence comprising a sequence, e.g., a crRNA sequence, that has sufficient complementarity with a target gene sequence to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease and the guide RNA, e.g., sgRNA. In some embodiments, the cleavage site is situated at a site within the target gene that is homologous to the sequence of the guide RNA, e.g., sgRNA. In some embodiments, the cleavage site is situated approximately 3 nucleotides upstream of the PAM or TAM sequence. In some embodiments, the cleavage site is situated approximately 3 nucleotides upstream of the juncture between the guide RNA and the PAM or TAM sequence. In some embodiments, the cleavage site is situated 3 nucleotides upstream of the PAM or TAM sequence. In some embodiments, the cleavage site is situated 4 nucleotides upstream of the PAM or TAM sequence. In some embodiments, the cleavage site is situated approximately 25 nucleotides upstream from the PAM or TAM sequence. In some embodiments, the cleavage site is situated approximately 23 nucleotides upstream from the PAM or TAM sequence. In some embodiments, the cleavage site is situated approximately 19 nucleotides upstream from the PAM or TAM sequence. In some embodiments, the cleavage site is situated approximately 18 nucleotides upstream from the PAM or TAM sequence. In some embodiments, the cleavage site is situated approximately 12 nucleotides upstream from the PAM or TAM sequence. In some embodiments, the cleavage site is situated approximately 8 nucleotides upstream from the PAM or TAM sequence.

In some embodiments, the genome editing complex capable of inducing a DSB comprises a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fokl nuclease domain. In some embodiments, the TAL effector DNA binding domain is engineered to target a specific target sequence, e.g., a portion of a target gene that includes a cleavage site.

In some embodiments, the fusion protein is a zinc finger nuclease (ZFN) comprising a zinc finger DNA binding domain and a DNA cleavage domain. In some embodiments, the DNA cleavage domain is a Fokl nuclease domain. In some embodiments, the zinc finger DNA binding domain is engineered to target a specific target sequence, e.g., a portion of a target gene, that includes a cleavage site, such as the targeting sequence.

In some embodiments, genome editing can be accomplished by introducing into a cell a genome editing complex of the disclosure. For example, introducing into a cell a genome editing complex capable of inducing a SSB at a cleavage site within the sense strand and an SSB at a cleavage site within the antisense strand of an endogenous target gene in the cell.

In some embodiments, the cleavage site in the sense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the antisense strand. In some embodiments, the cleavage site in the antisense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the sense strand. In some embodiments, the cleavage site in the sense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the antisense strand. In some embodiments, the cleavage site in the antisense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the sense strand.

In some embodiments, the genome editing complex capable of inducing an SSB at a cleavage site within the sense strand and an SSB at a cleavage site within the antisense strand comprise a recombinant nuclease. In some embodiments, the recombinant nuclease includes a recombinant nuclease that induces the SSB in the sense strand, and a recombinant nuclease that induced the SSB in the antisense strand, and both of which recombinant nucleases are referred to as the recombinant nuclease. Accordingly, in some embodiments, the method involves introducing into a cell the genome editing complex comprising a recombinant nuclease for inducing an SSB at a cleavage site in the sense strand and an SSB at a cleavage site in the antisense strand within an endogenous target gene in the cell. Although, in some embodiments, it is described that the recombinant nuclease induces an SSB in the antisense strand an SSB in the sense strand, it is to be understood that this includes situations where two of the same recombinant nucleases is used, such that one of the recombinant nucleases induces the SSB in the sense strand and the other recombinant nuclease induces the SSB in the antisense strand. In some embodiments, the recombinant nuclease that induces the SSB lacks the ability to induce a DSB by cleaving both strands of double stranded DNA.

In some embodiments, the genome editing complex capable of inducing an SSB comprises a recombinant nuclease and a first guide RNA, e.g., a first sgRNA, and a second guide RNA, e.g., a second sgRNA.

In some embodiments, the genome editing complex capable of inducing an SSB at a cleavage site within the sense strand and an SSB at a cleavage site within the antisense strand comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fokl nuclease domain. In some embodiments, the TAL effector DNA binding domain is engineered to target a specific target sequence, e.g., a portion of a target gene that includes a cleavage site. In some embodiments, the fusion protein is a zinc finger nuclease (ZFN) comprising a zinc finger DNA binding domain and a DNA cleavage domain. In some embodiments, the DNA cleavage domain is a Fokl nuclease domain. In some embodiments, the zinc finger DNA binding domain is engineered to target a specific target sequence, e.g., a portion of a target gene that includes a cleavage site, such as the targeting sequence.

In some embodiments, the genome editing complex capable of inducing an SSB at a cleavage site within the sense strand and an SSB at a cleavage site within the antisense strand involve use of the CRISPR/Cas gene editing system. In some embodiments the genome editing complex comprises a recombinant nuclease, such as Mad7 or Cas9.

In some embodiments, the genome modifying entity is targeted to the cleavage site by interacting with a guide RNA, e.g., a first guide RNA, such as a first sgRNA, or a second guide RNA, such as a second sgRNA, that hybridizes to a DNA sequence on the sense strand or the antisense strand that immediately precedes a PAM or TAM sequence. In some embodiments, the genome modifying entity is targeted to the cleavage site on the sense strand by interacting with a first guide RNA, e.g., first sgRNA, that hybridizes to a sequence on the sense strand that immediately precedes a PAM sequence. In some embodiments, the genome modifying entity is targeted to the cleavage site on the antisense strand by interacting with a second guide RNA, e.g., second sgRNA, that hybridizes to a sequence on the antisense strand that immediately precedes a PAM sequence. In some embodiments, the first guide RNA, e.g., first sgNA, that is specific to the sense strand of a target gene of interest is used to target a genome modifying entity to induce an SSB at a cleavage site within the sense strand of the target gene. In some embodiments, the first guide RNA, e.g., first sgNA, that is specific to the antisense strand of a target gene of interest is used to target the genome modifying entity to induce an SSB at a cleavage site within the antisense strand of the target gene. In some embodiments, the second guide RNA, e.g., second sgNA, that is specific to the sense strand of a target gene of interest used to target the genome modifying entity to induce an SSB at a cleavage site within the sense strand of the target gene. In some embodiments, the second guide RNA, e.g., second sgNA, that is specific to the antisense strand of a target gene of interest is used to target the genome modifying entity to induce an SSB at a cleavage site within the antisense strand of the target gene. In some embodiments, the first guide RNA, e.g., first sgNA, that is specific to the sense strand of a target gene of interest is used to target the genome modifying entity to induce an SSB at a cleavage site within the sense strand of the target gene; and the second guide RNA, e.g., second sgNA, that is specific to the antisense strand of a target gene of interest is used to target the genome modifying entity to induce an SSB at a cleavage site within the antisense strand of the target gene. In some embodiments, the genome modifying entity is, e.g., Mad7 or Cas9. In some embodiments, the first guide RNA, e.g., first sgNA, that is specific to the antisense strand of a target gene of interest is used to target the genome modifying entity to induce an SSB at a cleavage site within the antisense strand of the target gene; and the second guide RNA, e.g., second sgNA, that is specific to the sense strand of a target gene of interest is used to target the genome modifying entity to induce an SSB at a cleavage site within the sense strand of the target gene.

In some embodiments, the cleavage site is situated at a site within the target gene that is homologous to a sequence comprised within the guide RNA, e.g., sgRNA. In some embodiments, the cleavage site of the sense strand is situated at a site within the sense strand of the target genomic locus that is homologous to a sequence comprised within the first guide RNA, e.g., the first sgRNA. In some embodiments, the cleavage site of the antisense strand is situated at a site within the antisense strand of the target genomic locus that is homologous to a sequence comprised within the first guide RNA, e.g., the first sgRNA. In some embodiments, the cleavage site of the sense strand is situated at a site within the sense strand of the target genomic locus that is homologous to a sequence comprised within the second guide RNA, e.g., the second sgRNA. In some embodiments, the cleavage site of the antisense strand is situated at a site within the antisense strand of the target genomic locus that is homologous to a sequence comprised within the second guide RNA, e.g., the second sgRNA. In some embodiments, the cleavage site of the sense strand is situated at a site within the sense strand of the target genomic locus that is homologous to a sequence comprised within the first guide RNA, e.g., the first sgRNA; and the cleavage site of the antisense strand is situated at a site within the antisense strand of the target genomic locus that is homologous to a sequence comprised within the second guide RNA, e.g., the second sgRNA. In some embodiments, the cleavage site of the antisense strand is situated at a site within the antisense strand of the target genomic locus that is homologous to a sequence comprised within the first guide RNA, e.g., the first sgRNA; and the cleavage site of the sense strand is situated at a site within the sense strand of the target genomic locus that is homologous to a sequence comprised within the second guide RNA, e.g., the second sgRNA. In some embodiments, the cleavage site of the antisense strand is situated at a site within the antisense strand of the target genomic locus that is homologous to a sequence comprised within the second guide RNA, e.g., the second sgRNA; and the cleavage site of the sense strand is situated at a site within the sense strand of the target genomic locus that is homologous to a sequence comprised within the first guide RNA, e.g., the first sgRNA.

In some embodiments, the sense strand comprises the targeting sequence, and the targeting sequence includes a protospacer adjacent motif (PAM) or a target adjacent motif or transposon-associated motif (TAM) sequence. In some embodiments, the sense strand comprises the targeting sequence, and the targeting sequence includes a PAM or TAM sequence; and the antisense strand comprises a sequence that is complementary to the targeting sequence and includes a PAM or TAM sequence. In some embodiments, the antisense strand comprises the targeting sequence, and the targeting sequence includes a PAM or TAM sequence. In some embodiments, the antisense strand comprises the targeting sequence, and the targeting sequence includes a PAM or TAM sequence; and the sense strand comprises a sequence that is complementary to the targeting sequence and includes a PAM or TAM sequence.

In some embodiments, the cleavage site on the sense strand and/or the antisense strand is situated approximately 3 nucleotides upstream of the PAM or TAM sequence. In some embodiments, the cleavage site on the sense strand and/or the antisense strand is situated approximately 3 nucleotides upstream of the juncture between the guide RNA and the PAM or TAM sequence. In some embodiments, the cleavage site on the sense strand and/or the antisense strand is situated 3 nucleotides upstream of the PAM or TAM sequence. In some embodiments, the cleavage site on the sense strand and/or the antisense strand is situated 4 nucleotides upstream of the PAM or TAM sequence.

In some embodiments, the PAM or TAM sequence that is recognized by a recombinant nuclease is in the sense strand. In some embodiments, the PAM or TAM sequence that is recognized by a recombinant nuclease is in the antisense strand. In some embodiments, the PAM or TAM sequence that is recognized by a recombinant nuclease is in the sense strand and is in the antisense strand. In some embodiments, the PAM or TAM sequence on the sense strand and the PAM or TAM sequence on the antisense strand are outwardly facing. In some embodiments, the PAM or TAM sequence on the sense strand and the PAM or TAM sequence on the antisense strand comprise the same nucleic acid sequence, which can be any PAM or TAM sequence disclosed herein or known in the art. In some embodiments, the PAM or TAM sequence on the sense strand and the PAM or TAM sequence on the antisense strand each comprise a different nucleic acid sequence, each of which can be any of the PAM or TAM sequences disclosed herein or known in the art.

As is known in the art, the PAM or TAM sequence that is recognized by a recombinant nuclease, such as Cas9 or Mad7, differs depending on the particular recombinant nuclease and the organism species it is from.

In some embodiments, the genome editing complex can be provided in lipid particles that contain a nuclease protein and the nuclease protein to be directly delivered to a target cell. Methods of delivering a nuclease protein include those as described, for example, in Cai et al., Elife, 2014, 3:e01911 and International patent publication No. WO2017068077. For instance, such lipid particles comprise one or more Cas protein(s), such as Cas9. In some embodiments, the nuclease protein (e.g., Cas, such as Cas9) is engineered as a chimeric nuclease protein with a viral structural protein (e.g., GAG) for packaging into the lipid particle (e.g., lentiviral vector particle, VLP, or gesicle). For instance, a chimeric Cas9-protein fusion with the structural GAG protein can be packaged inside a lipid particle. In some embodiments, the fusion protein is a cleavable fusion protein between (i) a viral structural protein (e.g., GAG) and (ii) a nuclease protein (e.g., Cas protein, such as Cas9). In some embodiments, the fusion protein is a cleavable fusion protein between (i) a viral matrix (MA) protein and (ii) a nuclease protein (e.g., Cas protein, such as Cas9). In some embodiments, the particle contains a nuclease protein (e.g., Cas protein, such as Cas 9) immediately downstream of the gag start codon.

In some embodiments, the lipid particles contain mRNA encoding a Cas nuclease (e.g., Cas9). In some embodiments, the lipid particles contain guide RNA (gRNA), such as a single guide RNA (sgRNA).

In some embodiments, the lipid particles (e.g., lentiviral particles, VLPs, or gesicles) containing a Cas nuclease (e.g., Cas9) further comprise, or are further complexed with, one or more CRISPR-Cas system guide RNA(s) for targeting a desired target gene. In some embodiments, the CRISPR guide RNAs are efficiently encapsulated in the CAS-containing lipid particles. In some embodiments, the lipid particles (e.g., lentiviral particles, VLPs, or gesicles) further comprise, or are further complexed with, a targeting nucleic acid.

b. Guide RNAs and Compositions Thereof

In some embodiments, one or more exogenous sequences, such as any of the exogenous sequences described above, may be inserted at endogenous proliferation genes or off-target cell marker genes using a nucleic acid-guided targeting entity, such as any of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising a gRNA and a Cas nuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.

In some embodiments, the exogenous sequences are inserted at endogenous proliferation genes or off-target cell marker genes using a nucleic acid-guided nuclease, such as a guide RNA-guided nuclease, e.g., a Cas protein or Mad7, as described herein. In such cases, the nucleic acid-guided nuclease is targeted to a cleavage site by interacting with a guide RNA, e.g., sgRNA, that hybridizes to a DNA sequence that immediately precedes a Protospacer Adjacent Motif (PAM) or target adjacent motif or transposon-associated motif (TAM) sequence. The guide RNA can bind to the nucleic acid-guided nuclease and target the nucleic acid-guided nuclease to a specific location within the target gene such as at a location within the sense strand or the antisense strand of the target gene that is or includes the cleavage site.

In general, a guide RNA, e.g., sgRNA, is any nucleotide sequence comprising a sequence, e.g., a crRNA sequence, that has sufficient complementarity with a target gene sequence to hybridize with the target gene sequence at the cleavage site and direct sequence-specific binding of the nucleic acid-guided nuclease to a portion of the target gene that includes the cleavage site. Full complementarity (100%) is not necessarily required, so long as there is sufficient complementarity to cause hybridization and promote formation of a complex, e.g., CRISPR complex, that includes the nucleic acid-guided nuclease and the guide RNA, e.g., sgRNA.

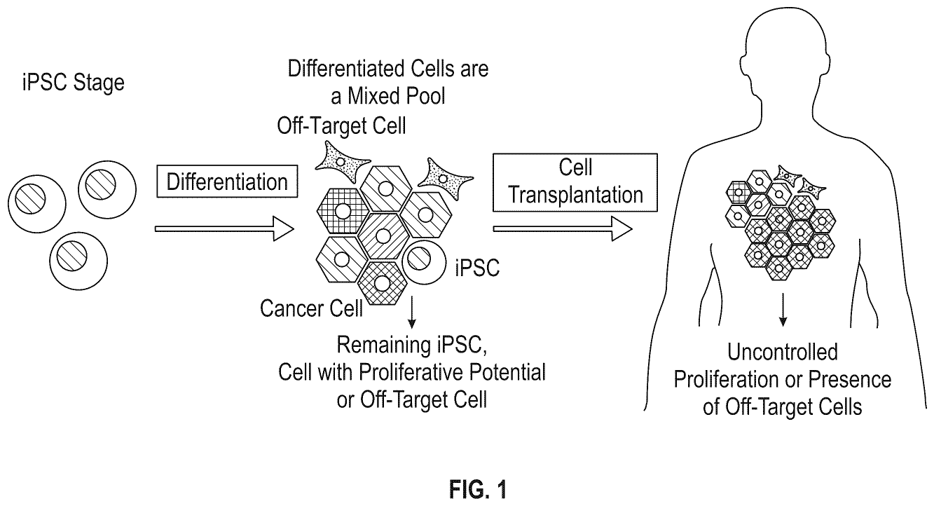

Methods for designing guide RNAs, e.g., sgRNAs, and their exemplary targeting sequences, e.g., crRNA sequences, can include those described in, e.g., International PCT Pub. Nos. WO2015/161276, WO2017/193107, and WO2017/093969. Exemplary guide RNA structures, including particular domains, are described in WO2015/161276, e.g., in FIGS. 1A-1G therein. Since a guide RNA is an RNA molecule, it will comprise the base uracil (U), while any DNA encoding the guide RNA molecule will comprise the base thymine (T). In some embodiments, the guide RNA, e.g., sgRNA, comprises a CRISPR targeting RNA sequence (crRNA) and a trans-activating crRNA sequence (tracrRNA). In some embodiments, the guide RNA, e.g., sgRNA, is an RNA comprising, from 5′ to 3′: a crRNA sequence and a tracrRNA sequence. In some embodiments, the crRNA and tracrRNA do not naturally occur together in the same sequence.

In some embodiments, the crRNA comprises a nucleotide sequence that is homologous, e.g., is at least 80%, 85%, 90%, 95%, 96%, 97%, 98%, or 99% homologous, or is 100% homologous, to a portion of the target genomic locus that includes the cleavage site. In some embodiments, the crRNA comprises a nucleotide sequence that is 100% homologous to a portion of the target genomic locus that includes the cleavage site. In some embodiments, the portion of the target genomic locus that includes the cleavage site is a portion of the sense strand of the target genomic locus that includes the cleavage site. In some embodiments, the portion of the target genomic locus that includes the cleavage site is a portion of the antisense strand of the target genomic locus that includes the cleavage site.

In some embodiments, more than one guide RNA (e.g., sgRNA) may be used to insert exogenous sequences at endogenous proliferation genes or off-target cell marker genes, such as a first and a second guide RNA. In some embodiments, the first guide RNA, e.g., the first sgRNA, and the second guide RNA, e.g., the second sgRNA, each comprise a crRNA and a tracrRNA. In some embodiments, each of the first guide RNA, e.g., first sgRNA, and the second guide RNA, e.g., second sgRNA, is an RNA comprising, from 5′ to 3′: a crRNA sequence and a tracrRNA sequence. In some embodiments, the crRNA and tracrRNA do not naturally occur together in the same sequence. In some embodiments, the sgRNA comprises a crRNA sequence that is homologous to a sequence in the target genomic locus that includes the cleavage site. In some embodiments, the first sgRNA comprises a crRNA sequence that is homologous to a sequence in the antisense strand of the target genomic locus that includes the cleavage site; and/or the second sgRNA comprises a crRNA sequence that is homologous to a sequence in the sense strand of the target genomic locus that includes the cleavage site.

In some embodiments, the crRNA sequence has 100% sequence identity to a sequence in the target genomic locus that includes the cleavage site. In some embodiments, the crRNA sequence of the first sgRNA has 100% sequence identity to a sequence in the sense strand of the target genomic locus that includes the cleavage site; and/or the crRNA sequence of the second sgRNA has 100% sequence identity to a sequence in the antisense strand of the target genomic locus that includes the cleavage site. In some embodiments, the crRNA sequence of the first sgRNA has 100% sequence identity to a sequence in the antisense strand of the target genomic locus that includes the cleavage site; and/or the crRNA sequence of the second sgRNA has 100% sequence identity to a sequence in the sense strand of the target genomic locus that includes the cleavage site.

Guidance on the selection of crRNA sequences can be found, e.g., in Fu Y et al., Nat Biotechnol 2014 (doi: 10.1038/nbt.2808) and Sternberg S H et al., Nature 2014 (doi: 10.1038/nature13011). Examples of the placement of crRNA sequences within the guide RNA, e.g., sgRNA, structure include those described in WO2015/161276, e.g., in FIGS. 1A-1G therein.

Reference to “the crRNA” is to be understood as also including reference to the crRNA of the first sgRNA and the crRNA of the second sgRNA, each independently. Thus, embodiments referring to “the crRNA” is to be understood as independently referring to embodiments of (i) the crRNA, (ii) the crRNA of the first sgRNA, and (iii) the crRNA of the second sgRNA. In some embodiments, the crRNA is 15-27 nucleotides in length, i.e., the crRNA is 15, 16, 17, 18, 19, 20, 21, 22, 23, 24, 25, 26, or 27 nucleotides in length. In some embodiments, the crRNA is 18-22 nucleotides in length. In some embodiments, the crRNA is 19-21 nucleotides in length. In some embodiments, the crRNA is 20 nucleotides in length. In some embodiments, the crRNA is 21 nucleotides in length.

In some embodiments, the crRNA is homologous to a portion of a target genomic locus that includes the cleavage site. In some embodiments, the crRNA is homologous to a portion of the sense strand of the target genomic locus that includes the cleavage site. In some embodiments, the crRNA is homologous to a portion of the antisense strand of the target genomic locus that includes the cleavage site. In some embodiments, the crRNA of the first sgRNA is homologous to a portion of the sense strand of the target genomic locus that includes the cleavage site; and the crRNA of the second sgRNA is homologous to a portion of the antisense strand of the target genomic locus that includes the cleavage site.

In some embodiments, the crRNA is homologous to a portion of the antisense strand of a target genomic locus that includes the cleavage site. In some embodiments, the crRNA is homologous to a portion of the sense strand of the target genomic locus that includes the cleavage site. In some embodiments, the crRNA of the first sgRNA is homologous to a portion of the antisense strand of the target genomic locus that includes the cleavage site; and the crRNA of the second sgRNA is homologous to a portion of the sense strand of the target genomic locus that includes the cleavage site.

In some embodiments, the crRNA is homologous to a portion of a target genomic locus that includes the cleavage site, and is 15-27 nucleotides in length, i.e., the crRNA is 15, 16, 17, 18, 19, 20, 21, 22, 23, 24, 25, 26, or 27 nucleotides in length. In some embodiments, the portion of the target genomic locus that includes the cleavage site is on the sense strand. In some embodiments, the portion of the target genomic locus that includes the cleavage site is on the antisense strand.

In some embodiments, the crRNA is homologous to a portion, i.e., sequence, in the sense strand or the antisense strand of the target genomic locus that includes the cleavage site and is immediately upstream of a PAM or TAM sequence.

In some embodiments, the tracrRNA sequence may be or comprise any sequence for tracrRNA that is used in any CRISPR/Cas system known in the art. Reference to “the tracrRNA” is to be understood as also including reference to the tracrRNA of the first sgRNA and the tracrRNA of the second sgRNA, each independently. Thus, embodiments referring to “the tracrRNA” is to be understood as independently referring to embodiments of (i) the tracrRNA, (ii) the tracrRNA of the first sgRNA, and (iii) the tracrRNA of the second sgRNA. Exemplary CRISPR/Cas systems, sgRNA, crRNA, and tracrRNA, and their manufacturing process and use include those described in, e.g., International PCT Pub. Nos. WO2015/161276, WO2017/193107 and WO2017/093969, and those described in, e.g., U.S. Patent Application Publication Nos. 20150232882, 20150203872, 20150184139, 20150079681, 20150073041, 20150056705, 20150031134, 20150020223, 20140357530, 20140335620, 20140310830, 20140273234, 20140273232, 20140273231, 20140256046, 20140248702, 20140242700, 20140242699, 20140242664, 20140234972, 20140227787, 20140189896, 20140186958, 20140186919, 20140186843, 20140179770, 20140179006, 20140170753, 20140093913, and 20140080216.

In some embodiments, the genome editing complex comprises a guide nucleic acid having a targeting domain that is complementary to at least one target sequence, optionally wherein the guide nucleic acid is a guide RNA (gRNA). In some cases, the genome editing complex is an RNA-guided nuclease, for example, a Cas nuclease and a guide RNA (CRISPR-Cas combination). In some embodiments, the CRISPR-Cas combination is a ribonucleoprotein (RNP) complex comprising the gRNA and the Cas nuclease. In some embodiments, the Cas nuclease is a Type II or Type V Cas protein. In some embodiments, the Cas nuclease is selected from Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, or Mad7.

In some embodiments, the cell described herein comprises a selection agent or a detection agent. In some embodiments, the selection agent is a kill switch. In some embodiments, the kill switch is selected from the group consisting of an inducible caspase 9 (iCasp9), cytosine deaminase (CDA), herpes simplex virus thymidine kinase (HSV-Tk), rapamycin-activated caspase 9 (rapaCasp9), chemically regulated-SH2-delivered inhibitory tail (CRASH-IT), nitroreductase (NTR), purine nucleoside phosphorylase (PNP), horseradish peroxidase, inducible MHC-I, inducible MHC-II, CCR4, CD16, CD19, CD20, CD30, EGFR, GD2, HER1, HER2, MUC1, PSMA, and RQR8. In some embodiments, the kill switch is iCasp9 or CDA. In some embodiments, the inducible MHC-I is selected from the group consisting of HLA-A, HLA-B, and HLA-C. In some embodiments, the inducible MHC-II is selected from the group consisting of HLA-DP, HLA-DQ, and HLA-DR. In some embodiments, the selection agent is a degron. In some embodiments, the detection agent is a cell surface protein. In some embodiments, the cell surface protein is selected from the group consisting of EGFR fused to a His-tag, RQRB fused to a His-tag, and CD47 fused to a His-tag. In some embodiments, the detection agent is a fluorescent protein. In some embodiments, the fluorescent protein is selected from the group consisting of: green fluorescent protein (GFP), enhanced green fluorescent protein (EGFP), yellow fluorescent protein (YFP), enhanced yellow fluorescent protein (EYFP), blue fluorescent protein (BFP), enhanced blue fluorescent protein (EBFP), cyan fluorescent protein (CFP), enhanced cyan fluorescent protein (ECFP), superfolder GFP, superfolder YFP, orange fluorescent protein, red fluorescent protein, small ultrared fluorescent protein, FMN-binding fluorescent protein, dsRed, qFP611, Dronpa, TagRFP, KFP, EosFP, IrisFP, Dendra, Kaede, KikGrl, emerald fluorescent protein, Azami Green, mWasabi, TagGFP, TurboGFP, AcGFP, ZsGreen, and T-Sapphire. In some embodiments, the detection agent is associated with a barcode. In some embodiments, the detection agent is a blood-detectable biomarker. In some embodiments, the detection agent is a secreted protein. In some embodiments, the secreted protein is detectable in vitro. In some embodiments, in vitro detection comprises detecting the secreted protein in cell culture medium collected from a cell culture comprising the cell described herein. In some embodiments, the secreted protein is detectable ex vivo. In some embodiments, ex vivo detection comprises detecting the secreted protein in a blood sample taken from an individual administered the cells described herein.

In some embodiments, the cell described herein is modified to express an engineered receptor. In some embodiments, the engineered receptor is a chimeric antigen receptor (CAR). In some embodiments, the CAR comprises an extracellular antigen binding domain specifically recognizing a target antigen; a transmembrane domain; and an intracellular signaling domain. In some embodiments, the cell is an immune cell. In some embodiments, the immune cell is a T cell, a B cell, or a natural killer (NK) cell.

In some embodiments, the cell described herein is a mammalian cell. In some embodiments, the mammalian cell is a human cell. In some embodiments, the cell is a stem cell-derived cell. In some embodiments, the stem cell-derived cell is derived from a cell selected from the group consisting of embryonic stem cell, induced pluripotent stem cell, multipotent stem cell, adult stem cell, hematopoietic stem cell, mesenchymal stem cell, endothelial stem cell, epithelial stem cell, adipose stem or progenitor cells, germline stem cells, lung stem or progenitor cells, mammary stem cells, olfactory adult stem cells, hair follicle stem cells, multipotent stem cells, amniotic stem cells, cord blood stem cells, neural stem, and progenitor cells. In some embodiments, the stem-cell derived cell is selected from the group consisting of a stem cell-derived beta cell, an alpha cell, and a delta cell. In some embodiments, the stem-cell derived cell is a stem cell-derived beta cell (SC-beta cell). In some embodiments, the stem-cell derived cell is a stem cell-derived beta islet cell. In some embodiments, the stem-cell derived cell is a stem cell-derived T cell. In some embodiments, the stem cell-derived T cell is selected from the group consisting of an ab T cell, dg T cell, helper/regulatory T cell, cytotoxic T cell, progenitor T cell (e.g., a progenitor T cell that is CD34+CD7+CD1a− or CD34+CD7+CD5+CD1a−), naive T cell, central memory T cell, effector T cell, terminal effector T cell, immature T cell, mature T cell, natural killer T cell, naive T cell, naive central memory T cell (TCM cell), effector memory T cell (TEM cell), and effector memory RA T cell (TEMRA cell). In some embodiments, the stem-cell derived cell is a stem cell-derived neural cell. In some embodiments, the stem cell-derived neural cell is selected from the group consisting of a glial cell, cerebral endothelial cell, neuron, ependymal cell, astrocyte, microglial cell, oligodendrocyte, and a Schwann cell. In some embodiments, the stem-cell derived cell is a stem cell-derived cardiac cell. In some embodiments, the stem cell-derived cardiac cell is selected from the group consisting of cardiomyocytes, nodal cardiomyocytes, conducting cardiomyocytes, working cardiomyocytes, cardiomyocyte precursors, cardiomyocyte progenitor cell, cardiac stem cell, and cardiac muscle cells.

In some embodiments, the cell described herein comprises modifications that (i) increase expression of one or more tolerogenic factors, and (ii) reduce expression of one or more major histocompatibility complex (MHC) class I molecules and/or one or more MHC class II molecules, wherein the increased expression of (i) and the reduced expression of (ii) is relative to a cell of the same cell type that does not comprise the modifications. In some embodiments, the one or more of the modifications in (ii) reduce expression of: a. one or more MHC class I molecules; b. one or more MHC class II molecules; or c. one or more MHC class I molecules and one or more MHC class II molecules. In some embodiments, the one or more modifications reduce expression of one or more molecules selected from the group consisting of B2M, TAP I, NLRC5, CIITA, HLA-A, HLA-B, HLA-C, HLA-DP, HLA-DM, HLA-DOA, HLA-DOB, HLA-DQ, HLA-DR, RFX5, RFXANK, RFXAP, NFY-A, NFY-B and/or NFY-C and any combination thereof. In some embodiments, the engineered cell does not express one or more molecules selected from the group consisting of B2M, TAP I, NLRC5, CIITA, HLA-A, HLA-B, HLA-C, HLA-DP, HLA-DM, HLA-DOA, HLA-DOB, HLA-DQ, HLA-DR, and combinations thereof. In some embodiments, the one or more tolerogenic factors is selected from the group consisting of CD47, A20/TNFAIP3, Cl-Inhibitor, CCL21, CCL22, CD16, CD16 Fc receptor, CD24, CD27, CD35, CD39, CD46, CD52, CD55, CD59, CD200, CR1, CTLA4-Ig, DUX4, FasL, H2-M3, HLA-C, HLA-E, HLA-E heavy chain, HLA-G, PD-L1, IDO1, IL-10, IL15-RF, IL-35, MANF, Mfge8, PD-1, PD-L1 or Serpinb9, and any combination thereof. In some embodiments, the one or more tolerogenic factors is selected from the group consisting of: a) CD47; b) HLA-E; c) CD24; d) PD-L1; e) CD46; f) CD55; f) CD59; h) CR1; i) MANF; j) A20/TNFAIP3; k) HLA-E and CD47; 1) CD24, CD47, PD-L1, and any combination thereof; m) HLA-E, CD24, CD47, and PD-L1, and any combination thereof; n) CD46, CD55, CD59, and CR1, and any combination thereof; o) HLA-E, CD46, CD55, CD59, and CR1, and any combination thereof; p) HLA-E, CD24, CD47, PDL1, CD46, CD55, CD59, and CR1, and any combination thereof; q) HLA-E and PDL1; r) HLA-E, PDL1, and A20/TNFA1P, and any combination thereof; sHLA-E, PDL1, and MANF, and any combination thereof; t) HLA-E, PDL1, A20/TNFAIP, and MANF, and any combination thereof; and u) CD47, PD-L1, HLA-E, HLA-G, CCL21, FASL, SERPINB9, CD200, MFGE8, and any combination thereof. In some embodiments, the engineered cell comprises modifications according to the following: (i) (a) reduces expression of MHC I and/or MHC II; (b) reduces expression of MIC-A and/or MIC-B; (c) increases expression of CD47, and optionally CD24 and PD-L1; and (d) increases expression of CD46, CD55, CD59 and CR1; (ii) (a) reduces expression of MHC class I molecule; (b) reduces expression of MIC-A and/or MIC-B; (c) reduces expression of TXNIP; (d) increases expression of PD-L1 and HLA-E; and (e) optionally increases expression of A20/TNFAIP3 and MANF; (iii) (a) increases expression of CCL21, PD-L1, FASL, SERPINB9, HLA-G, CD47, CD200, and MFGE8; and (b) reduces expression of a MICA and/or MICB; (iv) (a) reduces expression of MHC I and/or MHC II; and (b) increases expression of CD47; or (v) any of (i)-(iv) above further comprising modifications for increasing or decreasing expression of one or more additional genes, optionally reducing expression of B2M, TAP I, NLRC5, CIITA, HLA-A, HLA-B, HLA-C, HLA-DP, HLA-DM, HLA-DOA, HLA-DOB, HLA-DQ, HLA-DR, RFX5, RFXANK, RFXAP, NFY-A, NFY-B, NFY-C, CTLA-4, PD-1, IRF1, MIC-A, MIC-B, a protein that is involved in oxidative or ER stress, TRAC, TRB, CD142, ABO, CD38, PCDH11 Y, NLGN4Y and/or RHD, further optionally wherein proteins that are is involved in oxidative or ER stress include thioredoxin-interacting protein (TXNIP), PKR-like ER kinase (PERK), inositol-requiring enzyme 1α (IRE1α), and DJ-1 (PARK7).

In some embodiments, the cell provided herein comprises modifications that (i) increase expression of one or more tolerogenic factors selected from the group consisting of CD47, PD-L1, HLA-E, HLA-G, CCL21, FASL, SERPINB9, CD200, MFGE8, and any combination thereof, and (ii) reduce expression of one or more major histocompatibility complex (MHC) class I molecules and/or one or more MHC class II molecules, wherein the increased expression of (i) and the reduced expression of (ii) is relative to a cell of the same cell type that does not comprise the modifications. In some embodiments, the modification(s) that increase expression comprise increased surface expression, and/or the modifications that reduce expression comprise reduced surface expression, optionally wherein there is no detectable surface expression. In some embodiments, the modification that increases expression of the one or more tolerogenic factors comprises an exogenous polynucleotide encoding the one or more tolerogenic factors. In some embodiments, the one or more tolerogenic factors comprises CD47. In some embodiments, the one or more tolerogenic factors is CD47 and the exogenous polynucleotide encoding CD47 encodes a sequence of amino acids having at least 85% identity to the amino acid sequence of SEQ ID NO: 2, and reduces innate immune killing of the engineered primary cell. In some embodiments, the exogenous polynucleotide encoding CD47 encodes a sequence set forth in SEQ ID NO: 2. In some embodiments, the exogenous polynucleotide encoding the one or more tolerogenic factors is operably linked to a promoter. In some embodiments, the promoter is a constitutive promoter. In some embodiments, the promoter is selected from the group consisting of the CAG promoter, the cytomegalovirus (CMV) promoter, the EF1α promoter, the PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein barr virus (EBV) promoter, the Rous sarcoma virus (RSV) promoter and the UBC promoter. In some embodiments, the exogenous polynucleotide encoding CD47 is integrated into the genome of the engineered primary cell. In some embodiments, the exogenous polynucleotide is a multicistronic vector encoding the one or more tolerogenic factors and an additional transgene encoding a second transgene. In some embodiments, the integration is by non-targeted insertion into the genome of the engineered primary cell, optionally by introduction of the exogenous polynucleotide into the cell using a lentiviral vector. In some embodiments, the integration is by targeted insertion into a target genomic locus of the cell. In some embodiments, the target genomic locus is a a B2M gene locus, a CIITA gene locus, a MICA locus, a MICB locus, a TRAC gene locus, or a TRBC gene locus. In some embodiments, the target genomic locus is selected from the group consisting of: a CCR5 gene locus, a CXCR4 gene locus, a PPP1R12C (also known as AAVS1) gene, an albumin gene locus, a SHS231 locus, a CLYBL gene locus, a ROSA26 gene locus, ABO gene locus, F3 gene locus, FUT1 gene locus, HMGB1 gene locus, KDM5D gene locus, LRP1 gene locus, RHD gene locus, ROSA26 gene locus, and SHS231 gene locus. In some embodiments, the modification that reduces expression of one or more MHC class I molecules reduces one or more MHC class I molecules protein expression. In some embodiments, the modification that reduces expression of one or more MHC class I molecules is a modification that reduces expression of B-2 microglobulin (B2M). In some embodiments, the modification that reduces expression of one or more MHC class I molecules comprises reduced mRNA expression of B2M. In some embodiments, the modification that reduces expression of one or more MHC class I molecules comprises reduced protein expression of B2M. In some embodiments, the modification eliminates B2M gene activity. In some embodiments, the modification comprises inactivation or disruption of both alleles of the B2M gene. In some embodiments, the modification comprises inactivation or disruption of all B2M coding sequences in the cell. In some embodiments, the inactivation or disruption comprises an indel in the B2M gene. In some embodiments, the modification is a frameshift mutation or a deletion of a contiguous stretch of genomic DNA of the B2M gene. In some embodiments, the B2M gene is knocked out. In some embodiments, the modification that reduces expression of one or more MHC class I molecules is a modification that reduces expression of an HLA-A protein, an HLA-B protein, or HLA-C protein, optionally wherein a gene encoding said HLA-A protein, an HLA-B protein, or HLA-C protein is knocked out. In some embodiments, the modification that reduces expression of one or more MHC class II molecules reduces one or more MHC class II molecules protein expression. In some embodiments, the modification that reduces expression of one or more MHC class II molecules is a modification that reduces expression of CIITA. In some embodiments, the modification that reduces expression of one or more MHC class II molecules comprises reduced mRNA expression of CIITA. In some embodiments, the modification that reduces expression of one or more MHC class II molecules comprises reduced protein expression of CIITA. In some embodiments, the modification eliminates CIITA gene activity. In some embodiments, the modification comprises inactivation or disruption of both alleles of the CIITA gene. In some embodiments, the modification comprises inactivation or disruption of all CIITA coding sequences in the cell. In some embodiments, the inactivation or disruption comprises an indel in the CIITA gene. In some embodiments, the modification that reduces expression of one or more MHC class II molecules is a modification that reduces expression of an HLA-DP protein, an HLA-DR protein, or HLA-DQ protein, optionally wherein a gene encoding said HLA-DP protein, an HLA-DR protein, or HLA-DQ protein is knocked out. In some embodiments, the modification that reduces expression of the one or more MHC class I molecules and/or the one or more MHC class II molecules is by a genome-modifying protein. In some embodiments, the genome-modifying protein is associated with gene editing by a sequence-specific nuclease, a CRISPR-associated transposase (CAST), prime editing, or Programmable Addition via Site-specific Targeting Elements (PASTE). In some embodiments, the modification by the genome-modifying protein is nuclease-mediated gene editing. In some embodiments, the nuclease-mediated gene editing is by a zinc finger nuclease (ZFN), a TAL-effector nuclease (TALEN), or a CRISPR-Cas combination that targets the B2M gene, optionally wherein the Cas is Cas9. In some embodiments, the nuclease-mediated gene editing is by a CRISPR-Cas combination and the CRISPR-Cas combination comprises a guide RNA (gRNA) having a targeting domain that is complementary to at least one target site of an endogenous gene for reducing the expression of the one or more MHC class I molecules and/or one or more MHC class II molecules. In some embodiments, the CRISPR-Cas combination is a ribonucleoprotein (RNP) complex comprising the gRNA and a Cas protein. In some embodiments, the engineered primary cell is a human cell or an animal cell. In some embodiments, the engineered primary cell is a human cell. In some embodiments, the primary cell is a cell type that is exposed to the blood. In some embodiments, the engineered primary cell is a primary cell isolated from a donor subject. In some embodiments, the donor subject is healthy or is not suspected of having a disease or condition at the time the donor sample is obtained from the donor subject. In some embodiments, the engineered primary cell is selected from an islet cell, a beta islet cell, a pancreatic islet cell, an immune cell, a B cell, a T cell, a natural killer (NK) cell, a natural killer T (NKT) cell, a macrophage cell, an endothelial cell, a muscle cell, a cardiac muscle cell, a smooth muscle cell, a skeletal muscle cell, a dopaminergic neuron, a retinal pigmented epithelium cell, a hepatocyte, a thyroid cell, a skin cell, a glial progenitor cell, a neural cell, a cardiac cell, and a blood cell. In some embodiments, the engineered primary cell is an endothelial cell. In some embodiments, the engineered primary cell is an epithelial cell. In some embodiments, the engineered primary cell is a retinal pigmented epithelial cell. In some embodiments, the engineered primary cell is a T cell. In some embodiments, the engineered primary cell is an NK cell. In some embodiments, the engineered primary cell comprises a chimeric antigen receptor (CAR). In some embodiments, the engineered primary cell is an islet cell, optionally a beta islet cell. In some embodiments, the engineered primary cell is a hepatocyte. In some embodiments, the engineered primary cell is ABO blood group type O. In some embodiments, the engineered primary cell is Rhesus factor negative (Rh−).

In some embodiments, the cell described herein is capable of controlled killing of the engineered cell. In some embodiments, the engineered cell comprises a suicide gene or a suicide switch. In some embodiments, the suicide gene or the suicide switch induces controlled cell death in the presence of a drug or prodrug, or upon activation by a selective exogenous compound. In some embodiments, administration of an agent allows for depletion of an engineered cell of the population of engineered cells. In some embodiments, the agent recognizes the one or more tolerogenic factors on the surface of the engineered cell. In some embodiments, the engineered cell is engineered to express the one or more tolerogenic factors. In some embodiments, the one or more tolerogenic factors is CD47. In some embodiments, expression of a detection agent acts as a signal for administration of an exogenous kill switch directed against or specific to a tolerogenic agent. In some embodiments, the exogenous kill switch is an anti-CD47 antibody.

### D. Vectors

Vectors are nucleic acid (e.g., DNA) molecules that serve as vehicles to transfer foreign nucleic acid (e.g., DNA or a fragment thereof) into a host cell, in which the genetic material can be replicated and expressed. Vectors can be, for example, plasmids, viral vectors, cosmids, and artificial chromosomes. In the present disclosure, the vector may be an expression vector carrying a transgene encoding an agent (e.g., a detection agent or selection agent). In some embodiments, a vector as provided herein is a plasmid or a lentiviral vector.

Vectors may include additional components that are necessary for expression of a gene-of-interest (e.g., a transgene) in cells (e.g., multiple cloning site, promoter, regulatory elements, etc.). For example, a multiple cloning site acts as an insertion site for heterologous genes, reporters, additional gene regulatory elements, selectable markers, etc. Vectors may have one or more multiple cloning sites containing restriction sites. Furthermore, a promoter is a nucleic acid region that is upstream, or 5′, of a target gene, wherein transcription of the target gene is initiated at the promoter by a cell's transcription machinery. Promoters may be prokaryotic or eukaryotic, and furthermore can be expressed ubiquitously, expressed within specific cell types or subtypes, inducible, repressed, or otherwise cycled on/off in a controlled fashion or in response to cellular signaling pathway activity. Gene regulatory elements control the level and isoform type during gene expression. Regulatory elements may work at the transcriptional and/or the post-transcriptional stages. Gene regulatory elements may include promoters, enhancers, silencers, and insulators.

While not necessary for expression of a transgene encoded by a vector, a selectable marker can help identify and/or select for cells expressing the vector. Selectable markers may include antibiotic resistance genes, fluorescent proteins, toxins, etc. Selectable markers can permit positive selection (i.e., cells comprising the selectable marker are not killed) or negative selection (i.e., cells comprising the selectable marker are killed). Non-limiting examples of antibiotic resistance selectable markers known to those skill in the art include, for example, ampicillin, blasticidin, carbenicillin, chloramphenicol, hygromycin B, kanamycin, puromycin, spectinomycin, tetracycline, and zeocin.

Additional methods of inserting a transgene into a vector provided herein include recombineering, homologous recombination, standard restriction enzyme cloning, CRISPR systems, zinc-finger nuclease (ZFN) systems, TALENS, etc., as is known by one skilled in the art.

i. Elements for Insertion of Transgenes

The vector(s) of the present disclosure further may encode an enzyme recognition sequence, such as a recombinase, integrase, or meganuclease recognition sequence. In some embodiments, the vector comprises one or more recombination and/or cassette exchange sequences. These sites located within the vector of the present disclosure can then be leveraged to insert desired sequences, including large DNA payloads (e.g., >100 bp) using a recombinase, integrase, meganuclease, or HDR-mediated insertion or substitution through recognition of the enzyme recognition sequence.

In some embodiments, a vector of the present disclosure comprises one or more recombination sequences, such as any of Cre recombination sequences, Bxb1 recombination sequences, Flp recombination sequences, A118 recombination sequences, φC31 recombination sequences, R recombinase recombination sequences, Lambda recombination sequences, HK101 recombination sequences, pSAM2 recombination sequences, Beta-siz recombination sequences, CipH recombination sequences, ParA recombination sequences, Gamma-delta recombination sequences, TP901 recombination sequences, or any combination thereof. In some embodiments, the recombination sequences are used in a method of making any of the vectors described herein, wherein the method of making a vector comprises homologous recombination and/or recombineering of a transgene into a vector backbone, such as any vector backbone known in the art or described herein.

In some embodiments, a vector of the disclosure includes enzyme recognition sequences for the bacteriophage P1 Cre/lox system, e.g., Cre recombination sequences. The Cre protein is a 343 amino acid protein that has two domains: the larger carboxyl (catalytic) domain and the smaller amino domain. When expressed in a cell containing the target lox sites, these lox sites are recombined by the Cre enzyme. LoxP (locus of X-over P1) is a specific sequence of the bacteriophage P1 that is 34 bp and is targeted by the Cre enzyme. The site includes an asymmetric 8 bp sequence in between two sets of symmetric 13 bp sequences. The 13 bp sequences are palindromic but the 8 bp spacer sequence is not palindromic, thus giving the loxP sequence its directionality. In particular, loxP sites are directional such that inverted loxP sites on the same chromosome arm will cause an inversion of the DNA sequence located between the two loxP sites, whereas a direct repeat of loxP sites will cause a deletion event. LoxP sites being located on different chromosomes can cause translocation events to be catalysed upon Cre recombinase induction. When loxP sites are located in the original sequence and on a donor plasmid sequence, the donor sequence can be swapped with the original sequence in a process called recombinase-mediated cassette exchange (RMCE). In some embodiments, the Cre recombination sequences comprise one or more of loxP, lox511, loxN, and lox2272, as well as combinations thereof. The Cre recombinase enzyme is not found naturally within mammalian cells, such as any of the mammalian cells described herein. Thus, Cre recombinase can be provided to cells to induce recombination as a polypeptide or as a nucleic acid encoding the Cre recombinase. In cases where a nucleic acid encoding the Cre recombinase is provided, expression of Cre recombinase may be controlled by an inducible promoter, including but not limited to, for example, the pL, pBAD, Tet-on/Tet-Off, Lac switch, ecdysone, cumate, or tamoxifen inducible promoters.

In some embodiments, a vector of the present disclosure includes enzyme recognition sequences for the mycobacteriophage large serine recombinase Bxb1, e.g., Bxb1 recombination sequences. Bxb1 recombinase catalyzes site-specific recombination between its corresponding attP and attB recognition sites. Depending on the relative orientation of the attP and attB sites, the reaction can result in excision, inversion, or integration of sequences between the recognition sites to yield the product sites known as attL and attR. The attP and attB sites are each 39 bp and 34 bp in length, respectively. The catalyzed reaction is unidirectional and irreversible unless an excisionase is present. In some embodiments, the Bxb1 recombination sequences in a vector of the present disclosure comprise an attP or attB sequence. In some embodiments, the Bxb1 recombination sequences are selected from attP0, attP15, attB0, attB15, attP6, attP13, attB6, or attB13, as well as combinations thereof. In some embodiments, the Bxb1 recombination sequences comprise a minimal attP or attB sequence.

In some embodiments, a vector of the present disclosure includes enzyme recognition sequences for the S. cerevisiae recombinase, flippase (Flp), e.g., Flp recombination sequences. Flp recombines the sequences between the short flippase recognition target (FRT) sites, which are 34 bp long. The wild-type FRT F site includes an asymmetric 8 bp sequence in between two sets of 13 bp flanking arm sequences

(5′ GAAGTTCCTATTCTCTAGAAAGTATAGGAACTTC 3′.

In some embodiments, the Flp recombination sequences are selected from F, F3, F5, F10, F11, F12, F13, F14, F15, and F16, as well as combinations thereof.

In some embodiments, a vector of the present disclosure further comprises one or more regulatory element sequences. In some embodiments, the one or more regulatory element sequences comprise one or more promoter sequences, enhancer sequences, intron sequences, terminator sequences, translation initiation signal sequences, polyadenylation signal sequences, replication element sequences, RNA processing and export element sequences, transposon sequences, transposase sequences, insulator sequences, 5′UTR sequences, 3′UTR sequences, mRNA 3′ end processing sequences, boundary element sequences, locus control region (LCR) sequences, matrix attachment region (MAR) sequences, ubiquitous chromatin opening elements, linker sequences, secretion signal sequences, anchoring peptide sequences, localization signal sequences, fusion tag sequences, affinity tag sequences, chaperonin sequences, protease sequences, or posttranscriptional regulatory element sequences.

In some embodiments, a vector of the present disclosure comprises a promoter, such as any of a CAG promoter, cytomegalovirus (CMV) promoter, EF1α promoter, EF1α short promoter, PGK promoter, adenovirus late promoter, vaccinia virus 7.5K promoter, SV40 promoter, tk promoter of HSV, mouse mammary tumor virus (MMTV) promoter, LTR promoter of HIV, Epstein Barr virus (EBV) promoter, Rous sarcoma virus (RSV) promoter, UBC promoter, MoMuLV promoter, an avian leukemia virus promoter, actin promoter, myosin promoter, hemoglobin promoter, creatine kinase promoter, hybrid CMV enhancer/chicken (3-actin (CBA) promoter, or CBA hybrid intron (CBh) promoter, or any other suitable promoter known in the art.

In some embodiments, a vector of the present disclosure comprises a polyadenylation signal, such as any of Rb B-globin polyA, Rb a-globin polyA, Human Growth Hormone polyA, Human B-globin polyA, Bovine Growth Hormone polyA, or SV40 late polyA, or any other suitable polyadenylation signal known in the art.

In some embodiments, a vector of the present disclosure comprises a linker sequence, such as any of an internal ribosome entry site (IRES) sequence, a cleavable peptide sequence, a 2A peptide sequence, a F2A peptide sequence, a E2A peptide sequence, a P2A peptide sequence, a T2A peptide sequence, or a tPT2A peptide sequence, or any other suitable linker sequence known in the art.

In some embodiments, a vector of the present disclosure comprises one or more sequences encoding one or more selection markers. In some embodiments, the one or more selection markers comprise a fluorescent protein. In some embodiments, the fluorescent protein is selected from green fluorescent protein (GFP), enhanced green fluorescent protein (EGFP), yellow fluorescent protein (YFP), enhanced yellow fluorescent protein (EYFP), blue fluorescent protein (BFP), enhanced blue fluorescent protein (EBFP), cyan fluorescent protein (CFP), enhanced cyan fluorescent protein (ECFP), superfolder GFP, superfolder YFP, orange fluorescent protein, red fluorescent protein, small ultrared fluorescent protein, FMN-binding fluorescent protein, dsRed, qFP611, Dronpa, TagRFP, KFP, EosFP, IrisFP, Dendra, Kaede, KikGrl, emerald fluorescent protein, Azami Green, mWasabi, TagGFP, TurboGFP, AcGFP, ZsGreen, or T-Sapphire, or any suitable fluorescent protein known in the art. In some embodiments, fluorescent protein selection markers are screened using flow cytometry to select for engineered cells or population of cells (e.g., T cells, B cells, NK cells, islet cells such as beta cells, or hypoimmunogenic cells thereof). In other embodiments, fluorescent protein selection markers are screened using spectral imaging to select for engineered cells or populations of cells. Selection for engineered cells or populations of cells can include, for example, selecting and/or sorting for engineered cells or a population of engineered cells to expand and culture in vitro, such as prior to therapeutic administration as described further herein. In some instances, selection for engineered cells can include, for example, selecting and/or sorting for engineered cells or a population of engineered cells to administer to an individual (such as a patient, for example a human patient) in need thereof.

In some embodiments, a vector of the present disclosure comprises one or more sequences encoding one or more selection proteins. In some embodiments, the one or more selection proteins are selected from a membrane-bound selection molecule, a blasticidin selection protein, a hygromycin selection protein, a puromycin selection protein, a zeocin selection protein, a neomycin selection protein, a ganciclovir selection protein, a 5′-fluorocytosine selection protein, a G418 (geneticin) selection protein, or a cytosine deaminase selection protein, or any other suitable selection protein known in the art. In some embodiments, antibiotic resistance screening is used to select for engineered cells or population of cells. In some embodiments, membrane-bound selection molecules can include, for example, membrane-bound biotin mimetic peptides (BMPs) or biotin acceptor peptides (BAPs) (see, e.g., WO2012085911, hereby incorporated by reference in its entirety, for a list of BMPs and BAPs). In some embodiments, the membrane-bound selection molecule can include membrane peptides with mutated posttranslational modifications, for example glycosylation, acetylation, phosphorylation, nitrosylation, methylation, lipidation, etc. In some embodiments, membrane-bound selection molecules may include, for example, extracellular domain peptides of exogenous receptors, extracellular and transmembrane domain peptides of exogenous receptors, and/or full-length peptides of exogenous receptors that are not otherwise produced by the modified cell as described herein or population of such cells. In some embodiments, membrane-bound selection molecules are screened using flow cytometry to select for engineered cells or populations of cells. In some embodiments, membrane-bound selection molecules are screened using cytometry by time of flight (i.e., CyTOF) to select for engineered cells or populations of cells. In some embodiments, membrane-bound selection molecules are screened using spectral imaging to select for engineered cells or populations of cells. In some embodiments, the engineered cell or population of cells as described herein is sorted or selected by flow cytometry, CyTOF, or spectral imaging using antibodies that bind to membrane-bound selection molecules.

iii. Insertion of Transgene Sequences into Vectors

In some embodiments, transgenes, such as any of the transgenes described herein, may be inserted at a vector, such as any vector known in the art or described herein. For example, in some embodiments, the transgene (e.g., an agent and/or the genome editing complex) may be inserted into a vector sequence as described herein. Insertion of transgenes into a vector may be accomplished by various methods and agents, for example, using recombination-based methods, zinc finger nucleases, restriction enzymes, transcription activator-like effector nucleases (TALENs), Programmable Addition via Site-specific Targeting Elements (PASTE), nucleic acid-guided nuclease editing, and the like. In some embodiments, one or more transgenes may be inserted into a vector of the disclosure by one or more genetic rearrangements. In some embodiments, such genetic rearrangements are mediated by a site-specific recombinase. Thus, in some cases, a vector of the disclosure comprises, or is otherwise engineered to comprise a site-specific recombinase. In some embodiments, the site-specific recombinase comprises one or more of Cre, Bxb1, Flp, A118, φC31, R recombinase, Lambda, HK101, pSAM2, Beta-siz, CipH, ParA, Gamma-delta, or TP901.

In some embodiments, the site-specific recombinase is capable of targeting one or more recombination or cassette exchange sequences in the vector. In further embodiments, the site-specific recombinase is capable of inserting one or more transgenes into the vector. In some embodiments, the site-specific recombinase and the recombination or cassette exchange sequences are located on different vectors. In some embodiments, the site-specific recombinase and the recombination or cassette exchange sequences are located on the same vector.

In some embodiments, the vector comprises Cre recombination sequences, such as loxP, lox511, loxN, and lox2272, and the site-specific recombinase is Cre recombinase. In some embodiments, the vector comprises Bxb1 recombination sequences, such as attP and an attB sequence (e.g., any of attP0, attP15, attB0, attB15, attP6, attP13, attB6, and attB13, including minimal attP or attB sequences, such as any of SEQ ID NOs: 244, 277, 309, 367, and 370-374), and the site-specific recombinase is Bxb1 recombinase. In some embodiments, the vector comprises Flp recombination sequences, such as FRT, F, F3, F5, F10, F11, F12, F13, F14, F15, and F16, and the site-specific recombinase is Flp recombinase. In some embodiments, the vector comprises A118 recombination sequences, and the site-specific recombinase is A118. In some embodiments, the vector comprises φC31 recombination sequences, and the site-specific recombinase is φC31 recombinase.

In some embodiments, the transgene (e.g., an agent and/or the genome editing complex) to be inserted into a vector are encoded by a nucleic acid. In some embodiments, the nucleic acid encoding first transgene and/or the second transgene and the vector have matching recombination or cassette exchange sequences, such that the corresponding recombinase can insert the nucleic acid into a region of the vector by recombination. In some embodiments, the vector and the nucleic acid encoding the transgene (e.g., an agent and/or the genome editing complex) comprise Cre recombination sequences, such as loxP, lox511, loxN, and lox2272, and the site-specific recombinase is Cre recombinase. In some embodiments, the vector and the nucleic acid encoding the transgene (e.g., an agent and/or the genome editing complex) comprise Bxb1 recombination sequences, such as attP and an attB sequence (e.g., any of attP0, attP15, attB0, attB15, attP6, attP13, attB6, and attB13, including minimal attP or attB sequences, such as any of SEQ ID NOs: 244, 277, 309, 367, and 370-374), and the site-specific recombinase is Bxb1 recombinase. In some embodiments, the vector and the nucleic acid encoding the transgene (e.g., an agent and/or the genome editing complex) comprise Flp recombination sequences, such as FRT, F, F3, F5, F10, F11, F12, F13, F14, F15, and F16, and the site-specific recombinase is Flp recombinase. In some embodiments, the vector and the nucleic acid encoding the transgene (e.g., an agent and/or the genome editing complex) comprise A118 recombination sequences, and the site-specific recombinase is A118 recombinase. In some embodiments, the vector and the nucleic acid encoding the transgene (e.g., an agent and/or the genome editing complex) comprise φC31 recombination sequences, and the site-specific recombinase is φC31 recombinase.

Any of the vectors described herein can be inserted into a cell or a population of cells, such as any cell or population of cells described herein. Methods for the insertion or introduction of a vector of the present disclosure into cells are known to those of skill in the art and include, but are not limited to, lipid-mediated transfer (e.g., liposomes, including neutral and cationic lipids), electroporation, direct injection, cell fusion, particle bombardment, calcium phosphate co-precipitation, DEAE-dextran-mediated transfer and viral vector-mediated transfer.

### E. Types of Cells

The present disclosure provides cells, for example a population of cells, that have been engineered (hereafter interchangeably “cell” or “engineered cell”), e.g., to comprise one or more agents (e.g., a detection agent or selection agent), as well as compositions, methods, uses, and kits related thereto, as described herein. A cell according to the present disclosure may be any suitable cell known in the art, such as bacterial, mammalian, insect, fungal, or plant cells. The cells may be cell lines, primary cells, stem cells, differentiated cells derived or produced from such stem cells, hematopoietic stem cells, induced pluripotent stem cells, and the like. In some embodiments, the cell is a human cell. In some embodiments, the cell is a murine cell.

i. Primary and Induced Pluripotent Stem Cell-Derived Cells

In some embodiments, the present disclosure provides a cell (e.g., stem cell, induced pluripotent stem cell, differentiated cell derived or produced from such stem cell, hematopoietic stem cell, or primary cell), or population thereof, wherein the cell or population thereof comprises any of the vectors described herein. In some embodiments, the host cell is a mammalian cell. In some embodiments, the mammalian cell is a human cell. In some embodiments, the cell is in an immune cell. In some embodiments, the cell is a primary human cell.

In some embodiments, the cells (i.e., engineered cells or populations of cells) as provided herein are pluripotent stems cells or are cells differentiated from pluripotent stem cells. In some embodiments, the cells are primary cells.

The cell may be a vertebrate cell, for example, a mammalian cell, such as a human cell or a mouse cell. The cell may also be a vertebrate stem cell, for example, a mammalian stem cell, such as a human stem cell or a mouse stem cell. Preferably, the cell or stem cell is amenable to modification. Preferably, the cell or stem cell, or a cell derived from such a stem cell, has or is believed to have therapeutic value, such that the cell or stem cell or a cell derived or differentiated from such stem cell may be used to treat a disease, disorder, defect or injury in a subject in need of treatment for same.

In some embodiments, the cell is a stem cell or progenitor cell (e.g., iPSC, embryonic stem cell, hematopoietic stem cell, mesenchymal stem cell, endothelial stem cell, epithelial stem cell, adipose stem or progenitor cells, germline stem cells, lung stem or progenitor cells, mammary stem cells, olfactory adult stem cells, hair follicle stem cells, multipotent stem cells, amniotic stem cells, cord blood stem cells, or neural stem or progenitor cells). In some embodiments, the stem cells are adult stem cells (e.g., somatic stem cells or tissue specific stem cells). In some embodiments, the stem or progenitor cell is capable of being differentiated (e.g., the stem cell is totipotent, pluripotent, or multipotent). In some embodiments, the cell is isolated from embryonic or neonatal tissue. In some embodiments, the cell is a fibroblast, monocytic precursor, B cell, exocrine cell, pancreatic progenitor, endocrine progenitor, hepatoblast, myoblast, preadipocyte, progenitor cell, hepatocyte, chondrocyte, smooth muscle cell, K562 human erythroid leukemia cell line, bone cell, synovial cell, tendon cell, ligament cell, meniscus cell, adipose cell, dendritic cells, or natural killer cell. In some embodiments, the cell is manipulated (e.g., converted or differentiated) into a muscle cell, erythroid-megakaryocytic cell, eosinophil, iPSC, macrophage, T cell, islet beta-cell, neuron, cardiomyocyte, blood cell, endocrine progenitor, exocrine progenitor, ductal cell, acinar cell, alpha cell, beta cell, delta cell, PP cell, hepatocyte, cholangiocyte, or brown adipocyte. In some embodiments, the cell is a muscle cell (e.g., skeletal, smooth, or cardiac muscle cell), erythroid-megakaryocytic cell, eosinophil, iPSC, macrophage, T cell, islet beta-cell, neuron, cardiomyocyte, blood cell (e.g., red blood cell, white blood cell, or platelet), endocrine progenitor, exocrine progenitor, ductal cell, acinar cell, alpha cell, beta cell, delta cell, PP cell, hepatocyte, cholangiocyte, or white or brown adipocyte. In some embodiments, the cell is a hormone-secreting cell (e.g., a cell that secretes insulin, oxytocin, endorphin, vasopressin, serotonin, somatostatin, gastrin, secretin, glucagon, thyroid hormone, bombesin, cholecystokinin, testosterone, estrogen, or progesterone, renin, ghrelin, amylin, or pancreatic polypeptide), an epidermal keratinocyte, an epithelial cell (e.g., an exocrine secretory epithelial cell, a thyroid epithelial cell, a keratinizing epithelial cell, a gall bladder epithelial cell, or a surface epithelial cell of the cornea, tongue, oral cavity, esophagus, anal canal, distal urethra, or vagina), a kidney cell, a germ cell, a skeletal joint synovium cell, a periosteum cell, a bone cell (e.g., osteoclast, osteocyte, or osteoblast), a perichondrium cell (e.g., a chondroblast or chondrocyte), a cartilage cell (e.g., chondrocyte), a fibroblast, an endothelial cell, a pericardium cell, a meningeal cell, a keratinocyte precursor cell, a keratinocyte stem cell, a pericyte, a glial cell, an ependymal cell, a cell isolated from an amniotic or placental membrane, or a serosal cell (e.g., a serosal cell lining body cavities).

In some embodiments, the cell is a somatic cell. In some embodiments, the cells are derived from skin or other organs, e.g., heart, brain or spinal cord, liver, lung, kidney, pancreas, bladder, bone marrow, spleen, intestine, or stomach. The cells can be from humans or other mammals (e.g., rodent, non-human primate, bovine, or porcine cells).

In some embodiments, the cell is a mammalian cell, for example a primary cell or a cell line, selected from the group consisting of: islet cells, beta islet cells, pancreatic islet cells, immune cells, B cells, T cells, natural killer (NK) cells, natural killer T (NKT) cells, macrophage cells, endothelial cells, muscle cells, cardiac muscle cells, smooth muscle cells, skeletal muscle cells, dopaminergic neurons, retinal pigmented epithelium cells, optic cells, hepatocytes, thyroid cells, skin cells, glial progenitor cells, neural cells, cardiac cells, stem cells, hematopoietic stem cells, induced pluripotent stem cells (iPSCs), mesenchymal stem cells (MSCs), embryonic stem cells (ESCs), pluripotent stem cell (PSCs), blood cells, endothelial stem cells, epithelial stem cells, adipose stem or progenitor cells, germline stem cells, lung stem or progenitor cells, mammary stem cells, olfactory adult stem cells, hair follicle stem cells, multipotent stem cells, amniotic stem cells, cord blood stem cells, neural stem or progenitor cells, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, NS0, PerC6, Sp2/0, BHK, C127, and 211 A cells.

In some embodiments, the cell is an immune cell, such as, for example, an immune cell selected from the group consisting of: a natural killer (NK) cell, a natural killer T (NKT) cell, a T cell (e.g., CTL), a CD14+ cell, a dendritic cell, a PBMC cell, and any combination thereof. In some embodiments, the cell is an NK cell, a T cell (e.g., CTL), or a PBMC.

### Primary Cells

In some embodiments, the cell or population of cells as provided herein comprise cells derived from primary cells obtained or isolated from one or more individual subjects or donors. In some embodiments, the cells are derived from a pool of isolated primary cells obtained from one or more (e.g., two or more, three or more, four or more, five or more, ten or more, twenty or more, fifty or more, or one hundred or more) different donor subjects. In some embodiments, the primary cells isolated or obtained from the plurality of different donor subjects (e.g., two or more, three or more, four or more, five or more, ten or more, twenty or more, fifty or more, or one hundred or more) are pooled together in a batch and are engineered in accord with the provided methods.

In some embodiments, the primary cells are from a pool of primary cells from one or more donor subjects that are different than the recipient subject (e.g., the patient administered the cells). The primary cells can be obtained from 1, 2, 3, 4, 5, 6, 7, 8, 9, 10, 20, 50, 100 or more donor subjects and pooled together. The primary cells can be obtained from 1 or more, 2 or more, 3 or more, 4 or more, 5 or more, 6 or more, 7 or more, 8 or more, 9 or more, 10, or more 20 or more, 50 or more, or 100 or more donor subjects and pooled together. In some embodiments, the primary cells are harvested from one or a plurality of individuals, and in some instances, the primary cells or the pool of primary T cells are cultured in vitro. In some embodiments, the primary cells or the pool of primary T cells are engineered in accord with the methods provided herein.

In some embodiments, the methods include obtaining or isolating a desired type of primary cell (e.g., T cells, NK cells, NKT cells, endothelial cell, islet cell, beta islet cell, hepatocyte or other primary cells as described herein) from individual donor subjects, pooling the cells to obtain a batch of the primary cell type, and engineering the cells by the methods provided herein. In some embodiments, the methods include obtaining or isolating a desired type of primary cell (e.g., T cells, NK cells, endothelial cell, beta islet cell, hepatocyte or other primary cells as described herein), engineering cells of each of the individual donors by the methods provided herein, and pooling engineered cells or populations of cells of at least two individual samples to obtain a batch of engineered cells or populations of cells of the primary cell type.

In some embodiments, the primary cells are isolated or obtained from an individual or from a pool of primary cells isolated or obtained from more than one individual donor. The primary cells may be any type of primary cell described herein, including any described in herein. In some embodiments, the primary cells are selected from T cells, NK cells, beta islet cells, endothelial cells, epithelial cells such as RPE, thyroid, skin, or hepatocytes. In some embodiments, the primary cells from an individual donor or a pool of individual donors are engineered to contain and express any of the vectors described herein.

In some embodiments, the engineered cell or population of cells is a muscle cell (e.g., skeletal, smooth, or cardiac muscle cell), a skin cell, optic cells, immune cells, B cells, natural killer T cells, macrophages, erythroid-megakaryocytic cell, eosinophil, iPSC, macrophage, T cell, islet cluster, islet cell, beta-cell, neuron, cardiomyocyte, blood cell (e.g., red blood cell, white blood cell, or platelet), endocrine progenitor, exocrine progenitor, ductal cell, acinar cell, alpha cell, beta islet cell, delta cell, PP cell, hepatocyte, cholangiocyte, or white or brown adipocyte. In some embodiments, the cell is a hormone-secreting cell (e.g., a cell that secretes insulin, oxytocin, endorphin, vasopressin, serotonin, somatostatin, gastrin, secretin, glucagon, thyroid hormone, bombesin, cholecystokinin, testosterone, estrogen, or progesterone, renin, ghrelin, amylin, or pancreatic polypeptide), an epidermal keratinocyte, an epithelial cell (e.g., an exocrine secretory epithelial cell, a thyroid epithelial cell, a keratinizing epithelial cell, a gall bladder epithelial cell, or a surface epithelial cell of the cornea, tongue, oral cavity, esophagus, anal canal, distal urethra, or vagina), a kidney cell, a germ cell, a skeletal joint synovium cell, a periosteum cell, a bone cell (e.g., osteoclast, osteocyte, or osteoblast), a perichondrium cell (e.g., a chondroblast or chondrocyte), a cartilage cell (e.g., chondrocyte), a fibroblast, an endothelial cell, a pericardium cell, a meningeal cell, a keratinocyte precursor cell, a keratinocyte stem cell, a pericyte, a glial cell, an ependymal cell, a cell isolated from an amniotic or placental membrane, or a serosal cell (e.g., a serosal cell lining body cavities).

### Induced Pluripotent Stem Cells

In some embodiments, the cell or population thereof as provided herein are induced pluripotent stem cells or are engineered cells or populations of cells that are derived from or differentiated from induced pluripotent stem cells. The generation of mouse and human pluripotent stem cells (generally referred to as iPSCs; miPSCs for murine cells or hiPSCs for human cells) is generally known in the art. As will be appreciated by those skilled in the art, there are a variety of different methods for the generation of iPCSs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al., World J. Stem Cells 7(1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see, for example, Chapter 3 of the latter reference).

Generally, iPSCs are generated by the transient expression of one or more “reprogramming factors” in the host cell, usually introduced using episomal vectors. Under these conditions, small amounts of the cells are induced to become iPSCs (in general, the efficiency of this step is low, as no selection markers are used). Once the cells are “reprogrammed” and become pluripotent, they lose the episomal vector(s) and produce the factors using the endogenous genes.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g., fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4, and SOX2, are used. In other embodiments, four reprogramming factors, OCT4, KLF4, SOX2, and c-Myc, are used. In other embodiments, 5, 6, or 7 reprogramming factors can be selected from the group including, but not limited to: SOKMNLT, SOX2, OCT4 (POU5F1), KLF4, MYC, NANOG, LIN28, and SV40L T antigen. In general, these reprogramming factor genes are provided on episomal vectors such as are known in the art and commercially available.

In some embodiments, the hosts cells used for transfecting the one or more reprogramming factors are non-pluripotent stem cells. In general, as is known in the art, iPSCs are made from non-pluripotent cells such as, but not limited to, blood cells, fibroblasts, etc., by transiently expressing the reprogramming factors as described herein. In some embodiments, the non-pluripotent cells, such as fibroblasts, are obtained or isolated from one or more individual subjects or donors prior to reprogramming the cells. In some embodiments, iPSCs are made from a pool of isolated non-pluripotent stems cells, e.g., fibroblasts, obtained from one or more (e.g., two or more, three or more, four or more, five or more, ten or more, twenty or more, fifty or more, or one hundred or more) different donor subjects. In some embodiments, the non-pluripotent cells, such as fibroblasts, are isolated or obtained from a plurality of different donor subjects (e.g., two or more, three or more, four or more, five or more, ten or more, twenty or more, fifty or more, or one hundred or more), pooled together in a batch, reprogrammed as iPSCs, and engineered in accord with the provided methods.

In some embodiments, the iPSCs are derived from, such as by transiently transfecting one or more reprogramming factors into cells from a pool of non-pluripotent cells (e.g., fibroblasts) from one or more donor subjects that are different than the recipient subject (e.g., the patient administered the cells). The non-pluripotent cells (e.g., fibroblasts) to be induced to iPSCs can be obtained from 1, 2, 3, 4, 5, 6, 7, 8, 9, 10, 20, 50, 100 or more donor subjects and pooled together. The non-pluripotent cells (e.g., fibroblasts) can be obtained from 1 or more, 2 or more, 3 or more, 4 or more, 5 or more, 6 or more, 7 or more, 8 or more, 9 or more, 10, or more, 20 or more, 50 or more, or 100 or more donor subjects and pooled together. In some embodiments, the non-pluripotent cells (e.g., fibroblasts) are harvested from one or a plurality of individuals, and in some instances, the non-pluripotent cells (e.g., fibroblasts) or the pool of non-pluripotent cells (e.g., fibroblasts) are cultured in vitro and transfected with one or more reprogramming factors to induce generation of iPSCs. In some embodiments, the non-pluripotent cells (e.g., fibroblasts) or the pool of non-pluripotent cells (e.g., fibroblasts) are engineered or modified in accord with the methods provided herein. In some embodiments, the engineered iPSCs or a pool of engineered iPSCs are then subjected to a differentiation process for differentiation into any cells of an organism and tissue.

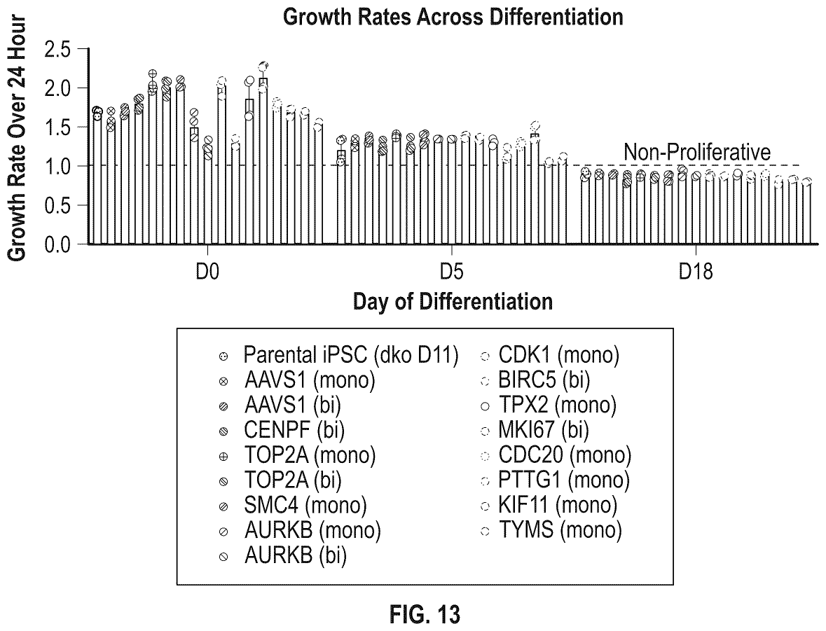

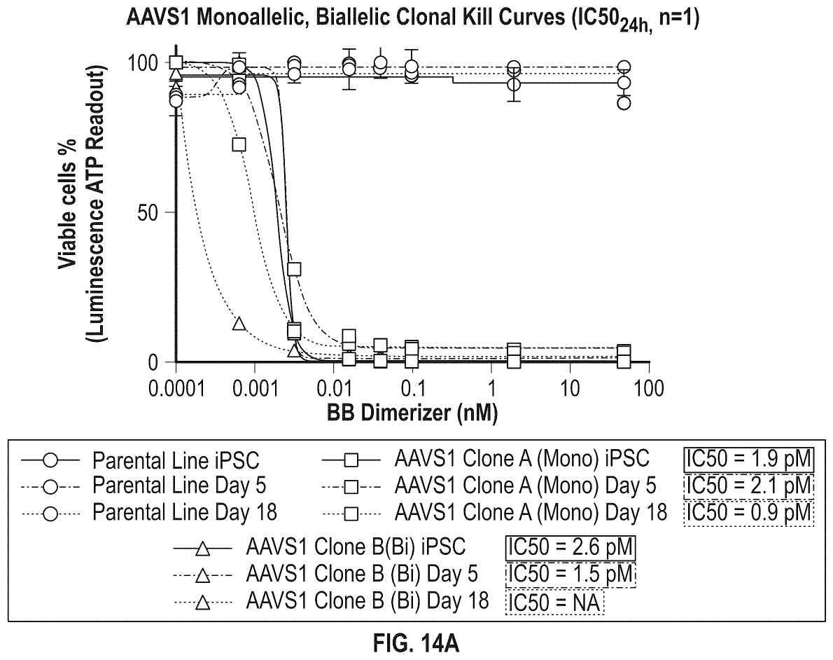

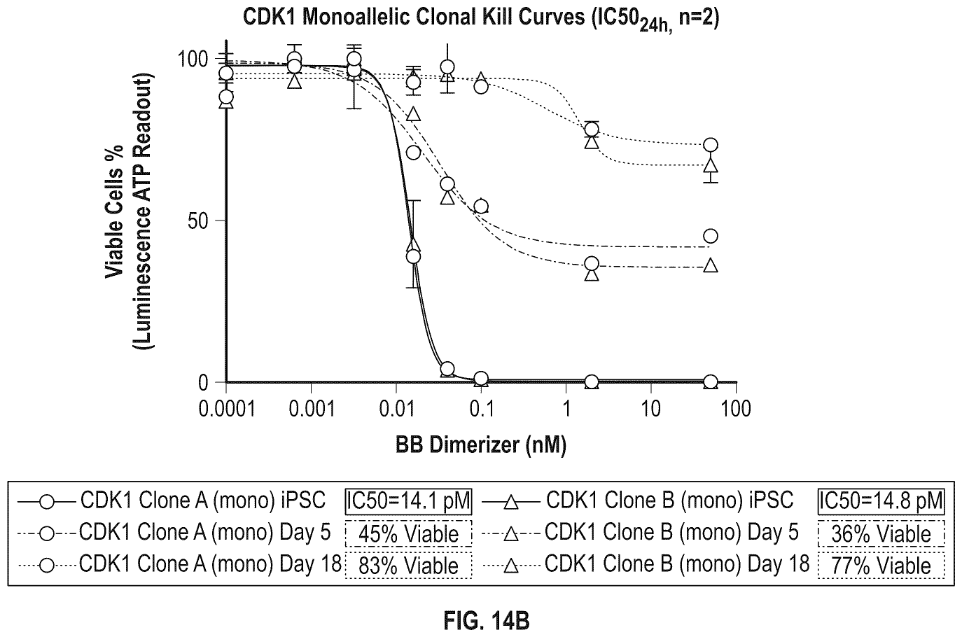

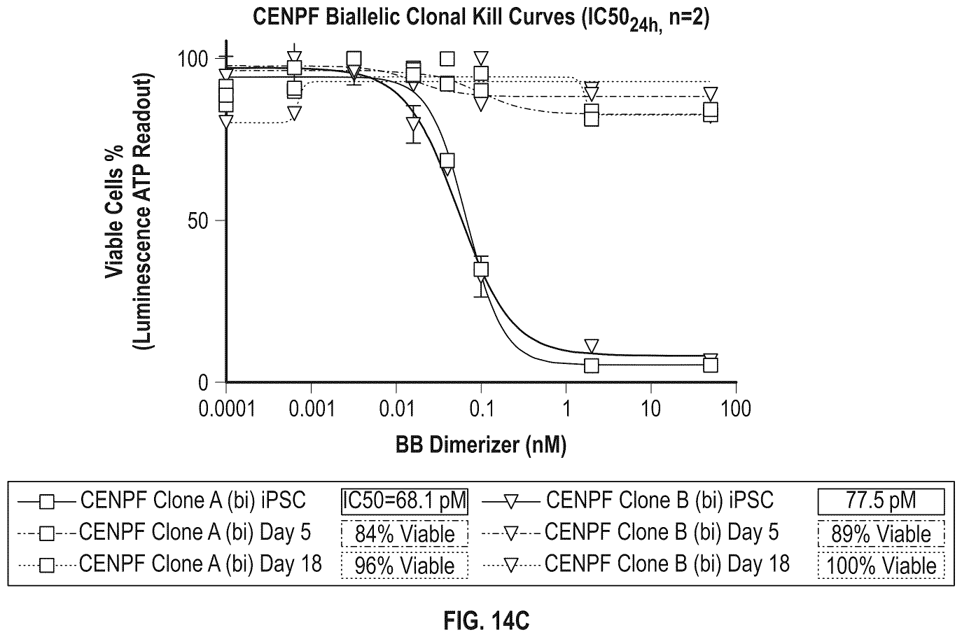

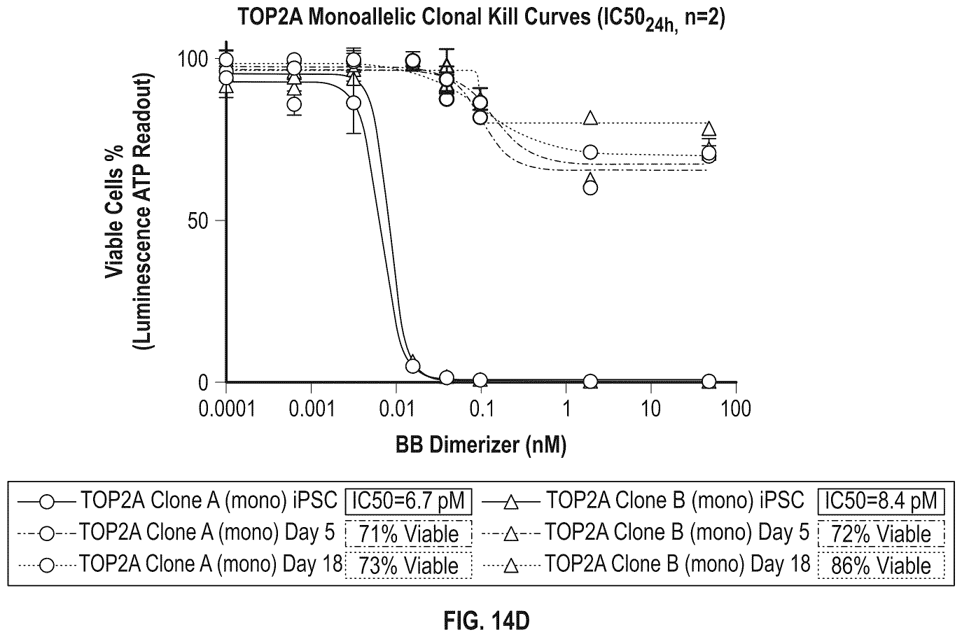

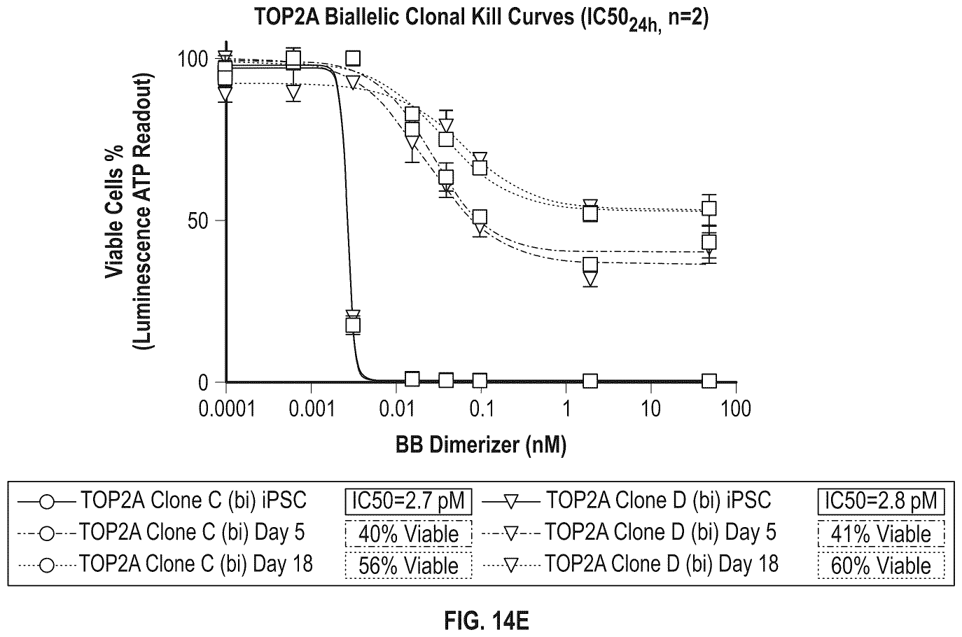

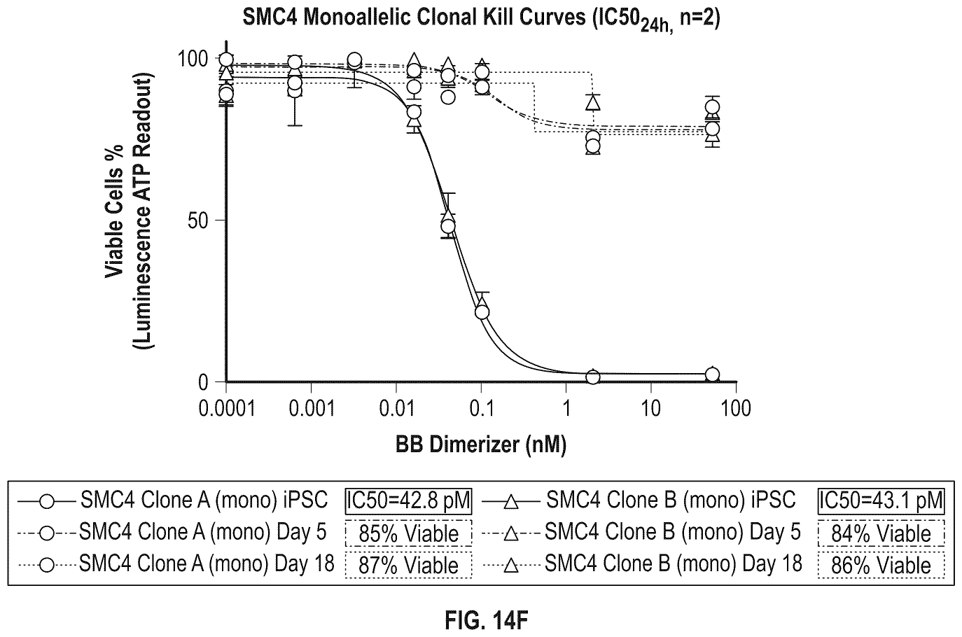

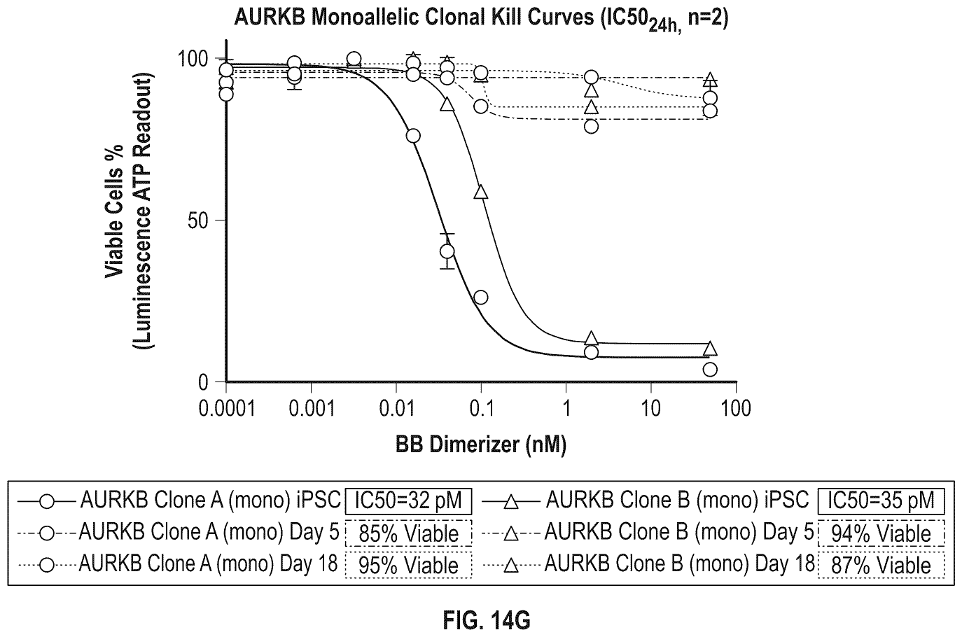

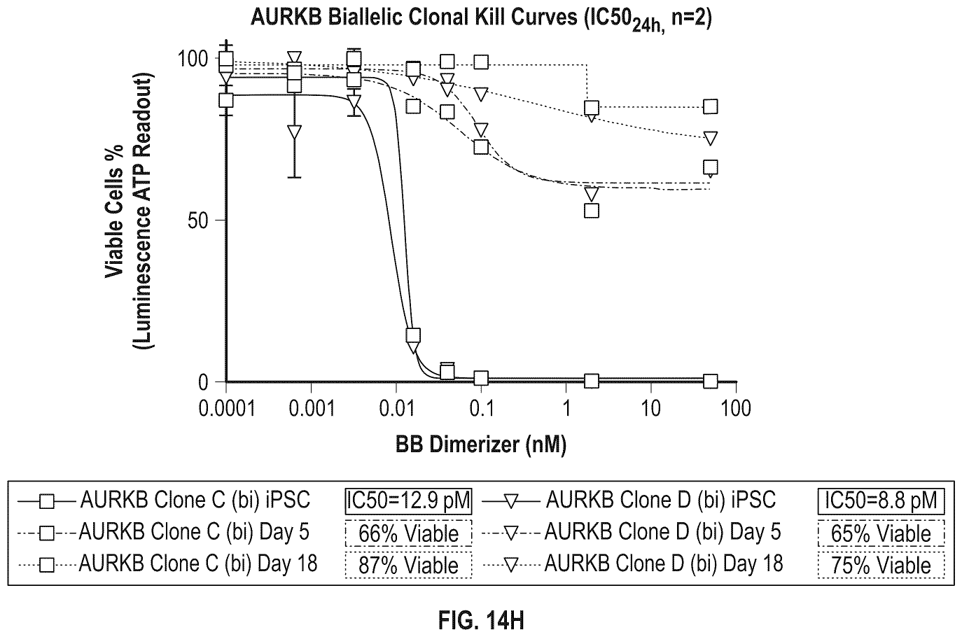

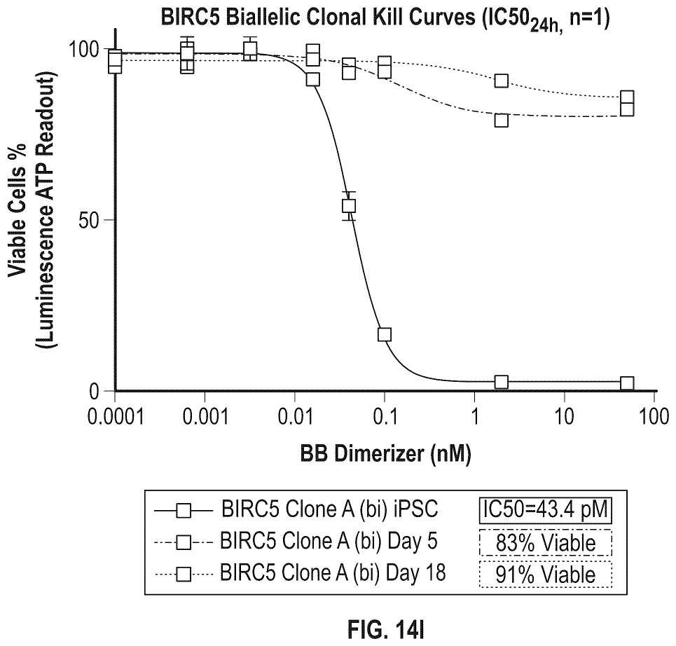

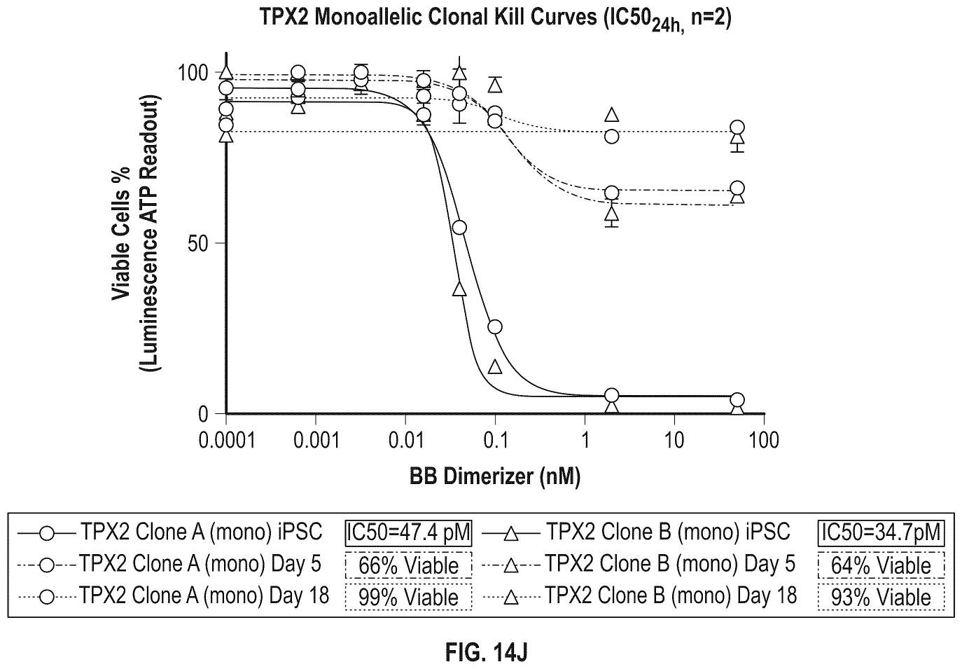

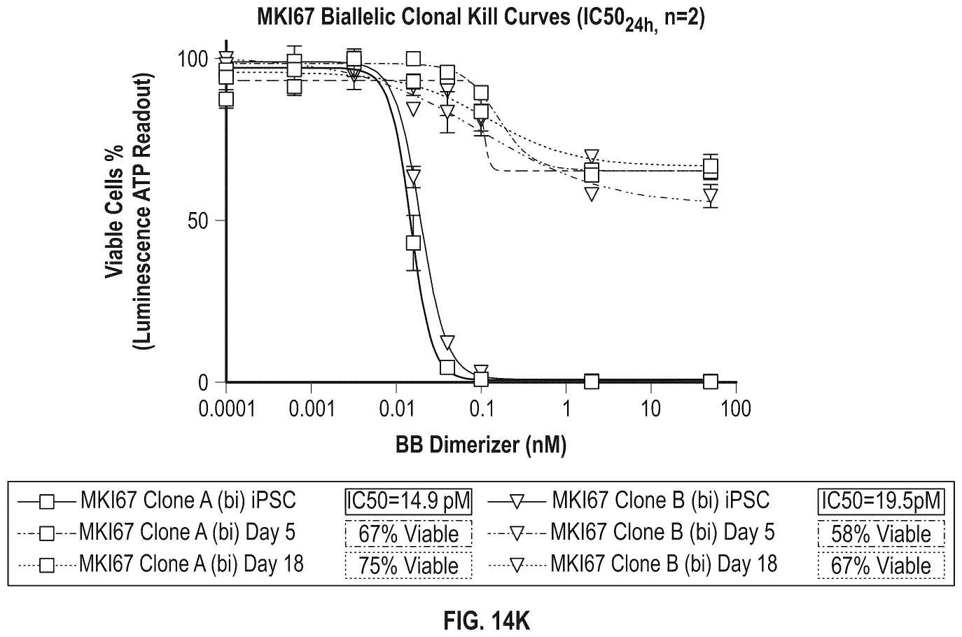

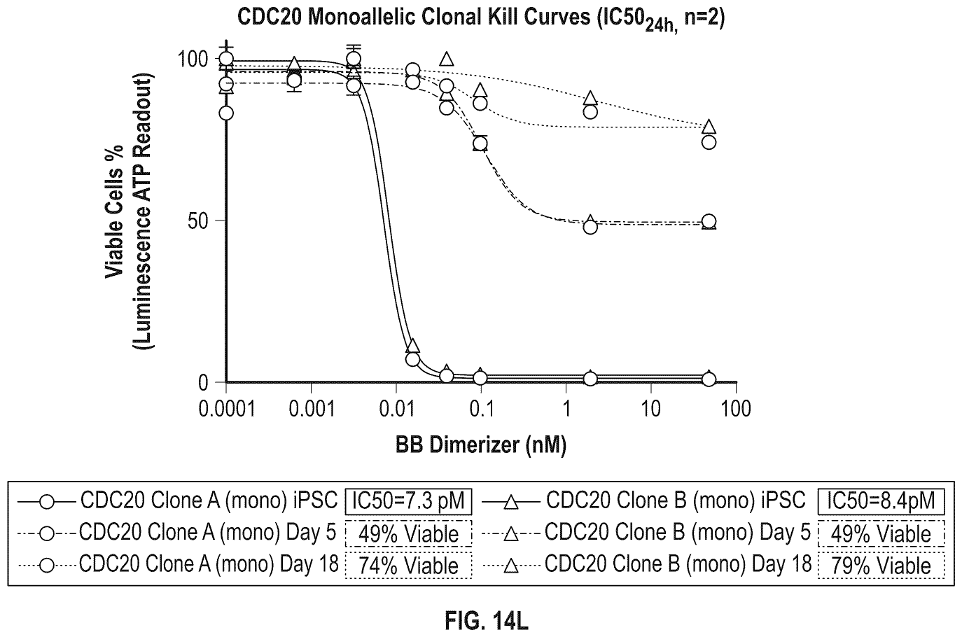

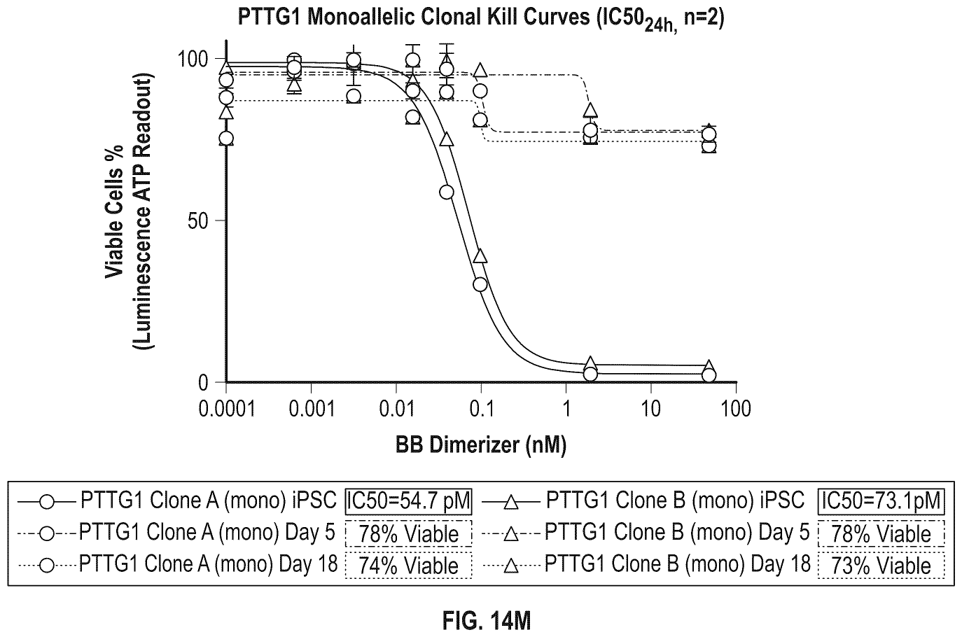

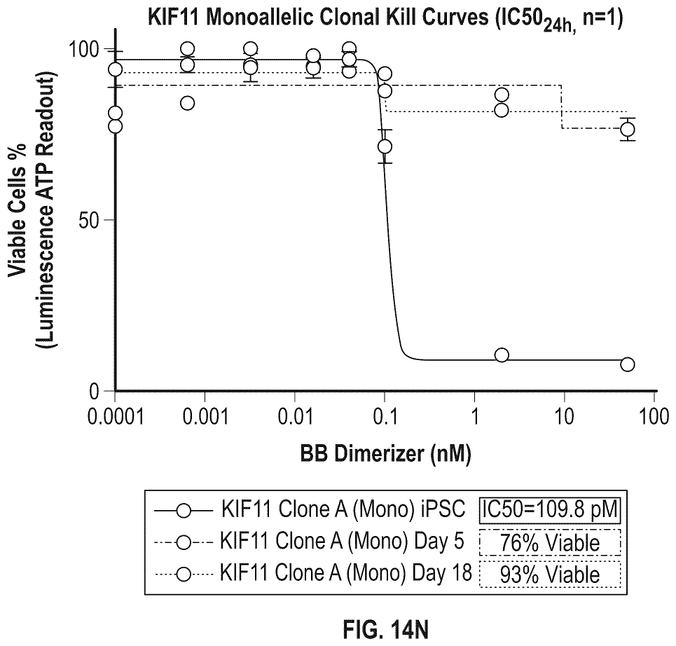

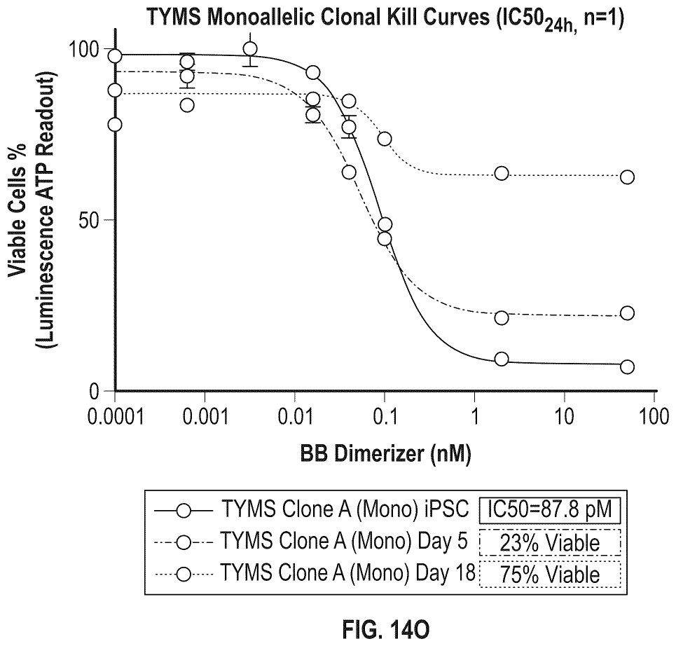

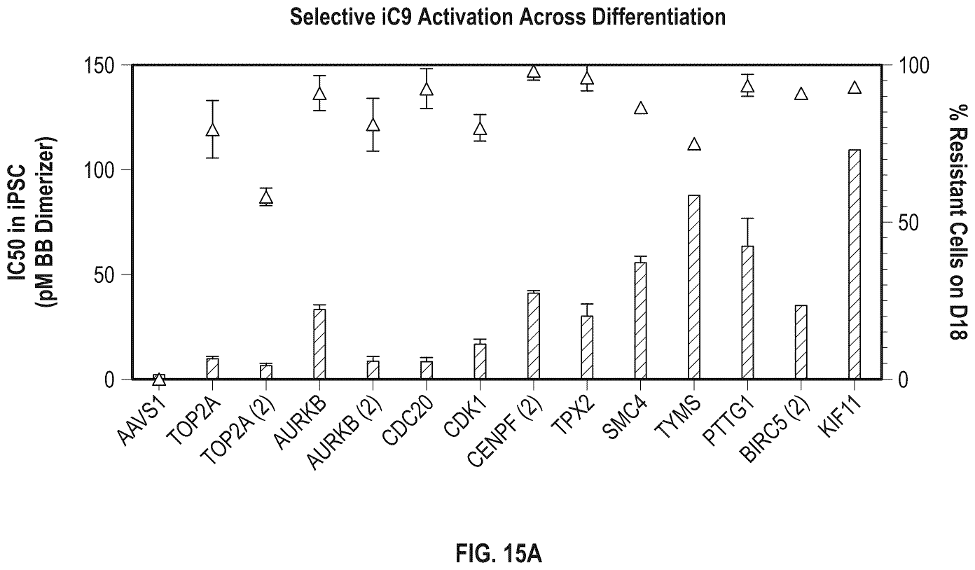

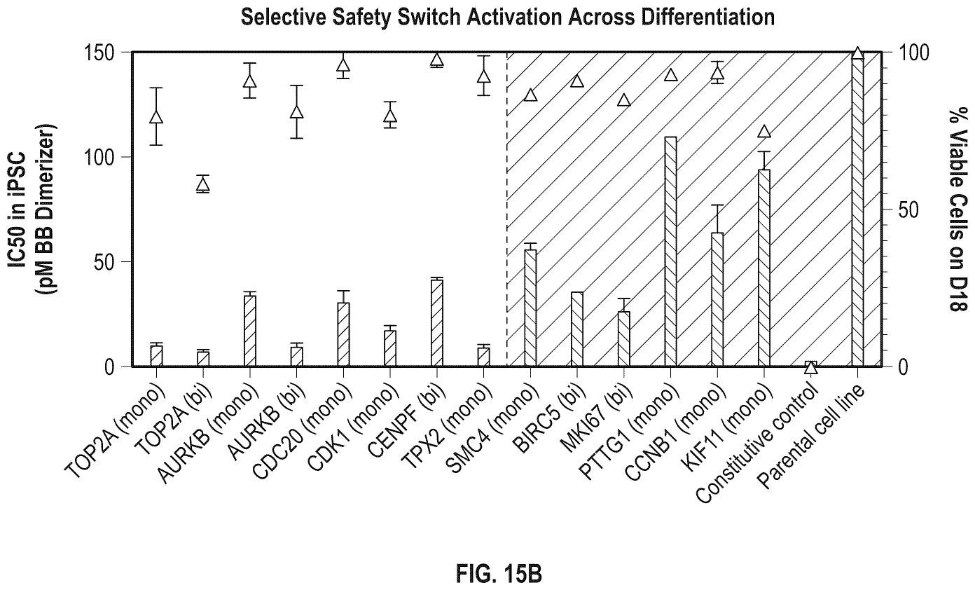

Once the engineered iPSCs cells have been generated, they may be assayed for their hypoimmunogenicity and/or retention of pluripotency as is described in WO2016183041 and WO2018132783. In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g., teratomas) that escape the host immune system. In some instances, hypoimmunogenic pluripotent cell derivatives are transduced to express luciferase and can then be followed using bioluminescence imaging. Similarly, the T cell and/or B cell response of the host animal to such cells are tested to confirm that the cells do not cause an immune reaction in the host animal. T cell responses can be assessed by Elispot, ELISA, FACS, PCR, or mass cytometry (CYTOF). B cell responses or antibody responses are assessed using FACS or Luminex. Additionally, or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of engineered or modified iPSCs is determined using an allogeneic humanized immunodeficient mouse model. In some instances, the engineered or modified iPSCs are transplanted into an allogeneic humanized NSG-SGM3 mouse and assayed for cell rejection, cell survival, and teratoma formation. In some instances, grafted engineered iPSCs or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

Similarly, the retention of pluripotency is tested in a number of ways. In one embodiment, pluripotency is assayed by the expression of certain pluripotency-specific factors as generally described herein and shown in FIG. 29 of WO2018132783. Additionally, or alternatively, the pluripotent cells are differentiated into one or more cell types as an indication of pluripotency.

Once the engineered pluripotent stem cells (engineered iPSCs) have been generated, they can be maintained in an undifferentiated state as is known for maintaining iPSCs. For example, the cells can be cultured on Matrigel using culture media that prevents differentiation and maintains pluripotency. In addition, they can be in culture medium under conditions to maintain pluripotency.

Any of the pluripotent stem cells described herein can be differentiated into any cells of an organism and tissue. In an aspect, provided herein are engineered cells or populations of cells that are differentiated into different cell types from iPSCs for subsequent transplantation into recipient subjects. Differentiation can be assayed as is known in the art, generally by evaluating the presence of cell-specific markers. As will be appreciated by those in the art, the differentiated engineered (e.g., hypoimmunogenic) pluripotent cell derivatives can be transplanted using techniques known in the art that depends on both the cell type and the ultimate use of these cells. Exemplary types of differentiated cells and methods for producing the same are described below. In some embodiments, the iPSCs may be differentiated to any type of cell described herein. In some embodiments, the iPSCs are differentiated into cell types selected from T cells, NK cells, beta islet cells, endothelial cells, epithelial cells such as RPE, thyroid, skin, or hepatocytes. In some embodiments, host cells such as non-pluripotent cells (e.g., fibroblasts) from an individual donor or a pool of individual donors are isolated or obtained, generated into iPSCs in which the iPSCs are then engineered to contain modifications (e.g., genetic modifications) described herein and then differentiated into a desired cell type.

### Islet Cells

In some embodiments, the engineered cells or populations of cells that are provided herein are islet cells, such as beta cells, primary beta cells, pancreatic islet cells or pancreatic beta cells. In some embodiments, the islet cells are isolated or obtained from one or more individual donor subjects, such as one or more individual healthy donor (e.g., a subject that is not known or suspected of, e.g., not exhibiting clinical signs of, a disease or infection). As will be appreciated by those skilled in the art, methods of isolating or obtaining islet cells from an individual can be achieved using known techniques.

In some embodiments, islet cells, such as beta cells, primary beta cells, pancreatic islet cells or pancreatic beta cells, are obtained (e.g., harvested, extracted, removed, or taken) from a subject or an individual. In some embodiments, islet cells are produced from a pool of islet cells such that the islet cells are from one or more subjects (e.g., one or more human including one or more healthy humans). In some embodiments, the pool of islet cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects.

In some embodiments, the engineered cells or populations of cells as provided herein are islet cells derived from engineered iPSCs that contain any of the vectors described herein and that are differentiated into beta islet cells. As will be appreciated by those skilled in the art, the methods for differentiation depend on the desired cell type using known techniques. In some embodiments, islet cells are derived from the engineered pluripotent cells described herein. Useful methods for differentiating pluripotent stem cells into islet cells are described, for example, in U.S. Pat. Nos. 9,683,215; 9,157,062; 8,927,280; U.S. Patent Pub. No. 2021/0207099; Hogrebe et al., Nat. Biotechnol., 2020, 38:460-470; and Hogrebe et al., Nat. Protoc., 2021, the contents of which are herein incorporated by reference in their entirety.

In some embodiments, the engineered pluripotent cells described herein are differentiated into beta-like cells or islet organoids. Pagliuca et al. (Cell, 2014, 159(2):428-39) and Vegas et al. (Nat Med, 2016, 22(3):306-11) report on the successful differentiation of β-cells from hiPSCs, the contents of which are incorporated herein by reference in their entirety.

In some embodiments, the method of producing engineered islet cells from engineered pluripotent cells by in vitro differentiation comprises: (a) culturing the engineered iPSCs in a first culture medium comprising one or more factors selected from the group consisting insulin-like growth factor, transforming growth factor, FGF, EGF, HGF, SHH, VEGF, transforming growth factor-β superfamily, BMP2, BMP7, a GSK inhibitor, an ALK inhibitor, a BMP type 1 receptor inhibitor, and retinoic acid to produce a population of immature pancreatic islet cells; and (b) culturing the immature islet cells in a second culture medium that is different than the first culture medium to produce engineered islet cells. In some embodiments, the GSK inhibitor is CHIR-99021, a derivative thereof, or a variant thereof. In some instances, the GSK inhibitor is at a concentration ranging from about 2 mM to about 10 mM. In some embodiments, the ALK inhibitor is SB-431542, a derivative thereof, or a variant thereof. In some instances, the ALK inhibitor is at a concentration ranging from about 1 μM to about 10 μM. In some embodiments, the first culture medium and/or second culture medium are absent of animal serum.

Differentiation is assayed as is known in the art, e.g., by evaluating the presence of beta cell-associated or specific markers, including but not limited to, insulin. Differentiation can also be measured functionally, such as measuring glucose metabolism, see generally Muraro et al., Cell Syst. 2016 Oct. 26; 3(4): 385-394.e3, hereby incorporated by reference in its entirety.

Additional descriptions of islet cells including for use in the present technology are found in WO2020/018615, the disclosure of which is herein incorporated by reference in its entirety.

In some embodiments, the engineered islet cells, such as primary beta islet cells isolated from one or more individual donors (e.g., healthy donors) or endothelial cells differentiated from iPSCs derived from one or more individual donors (e.g., healthy donors), are maintained in culture and in some cases expanded and/or cryopreserved. In certain embodiments, the engineered islet cells are cryopreserved.

Exemplary islet cell types include, but are not limited to, pancreatic islet progenitor cell, immature pancreatic islet cell, mature pancreatic islet cell, and the like.

In some embodiments, the islet cells engineered as disclosed herein, such as primary beta islet cells isolated from one or more individual donors (e.g., healthy donors) or beta islet cells differentiated from iPSCs derived from one or more individual donors (e.g., healthy donors), secrete insulin. In some embodiments, a pancreatic islet cell exhibits at least two characteristics of an endogenous pancreatic islet cell, for example, but not limited to, secretion of insulin in response to glucose, and expression of beta cell markers.

Exemplary beta cell markers or beta cell progenitor markers include, but are not limited to, c-peptide, Pdx1, glucose transporter 2 (Glut2), HNF6, VEGF, glucokinase (GCK), prohormone convertase (PC 1/3), Cdcp1, NeuroD, Ngn3, Nkx2.2, Nkx6.1, Nkx6.2, Pax4, Pax6, Ptfla, Isl1, Sox9, Sox17, and FoxA2.

In some embodiments, the islet cells, such as primary beta islet cells isolated from one or more individual donors (e.g., healthy donors) or beta islet cells differentiated from iPSCs derived from one or more individual donors (e.g., healthy donors), produce insulin in response to an increase in glucose. In various embodiments, the pancreatic islet cells secrete insulin in response to an increase in glucose. In some embodiments, the cells have a distinct morphology such as a cobblestone cell morphology and/or a diameter of about 17 pm to about 25 pm.

In some embodiments, the present disclosure is directed to engineered beta islet cells, such as primary beta islet cells isolated from one or more individual donors (e.g., healthy donors) or beta islet cells differentiated from iPSCs derived from one or more individual donors (e.g., healthy donors).

In some embodiments, the provided engineered islet cells evade immune recognition. In some embodiments, the engineered islet cells described herein, such as primary beta islet cells isolated from one or more individual donors (e.g., healthy donors) or beta islet cells differentiated from iPSCs derived from one or more individual donors (e.g., healthy donors), do not activate an immune response.

a. Engineered Islets

In some embodiments, the provided engineered islets, also includes a modification to modulate (e.g., increase) expression of one or more tolerogenic factor. In some embodiments, the modulation of expression of the tolerogenic factor (e.g., increased expression), and the modulation of expression of the one or more MHC class I molecules and/or one or more MHC class II molecules (e.g., reduced or eliminated expression) is relative to the amount of expression of said molecule(s) in a cell that does not comprise the modification(s), such as a control cell. In some embodiments, the modulation of expression is relative to the amount of expression of said molecule(s) in a wild-type cell. In some embodiments, the control or wild-type cell is an islet cell that has not been engineered with the modifications. In some embodiments, modulation of expression of the tolerogenic factor (e.g., increased expression), and the modulation of expression of the one or more MHC class I molecules and/or one or more MHC class II molecules (e.g., reduced or eliminated expression) is relative to the amount of expression of said molecule(s) in a control or wild-type cell of the same cell type that does comprise not the modification(s). In some embodiments, the control or wild-type cell does not express the one or more tolerogenic factor, the one or more MHC class I molecules, and/or the one or more MHC class II molecules. In some embodiments, it is understood that where the control or wild-type cell does not express the tolerogenic factor, the provided engineered islet cell includes a modification to overexpress the one or more tolerogenic factor or increase the expression of the one or more tolerogenic factor from 0%. It is understood that if the islet cell prior to the engineering does not express a detectable amount of the tolerogenic factor, then a modification that results in any detectable amount of an expression of the tolerogenic factor is an increase in the expression compared to the similar beta cell that does not contain the modifications.

In some embodiments, the provided engineered islets includes a modification to increase expression of one or more tolerogenic factors. In some embodiments, the tolerogenic factor is one or more of DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, and H2-M3 (including any combination thereof). In some embodiments, the tolerogenic factor is one or more of CD47, PD-L1, HLA-E or HLA-G, CCL21, FasL, Serpinb9, CD200, and Mfge8 (including any combination thereof). In some embodiments, the modification to increase expression of one or more tolerogenic factors is or includes increased expression of CD47. In some embodiments, the modification to increase expression of one or more tolerogenic factors is or includes increased expression of PD-L1. In some embodiments, the modification to increase expression of one or more tolerogenic factors is or includes increased expression of HLA-E. In some embodiments, the modification to increase expression of one or more tolerogenic factors is or includes increased expression of HLA-G. In some embodiments, the modification to increase expression of one or more tolerogenic factors is or includes increased expression of CCL21, PD-L1, FasL, Serpinb9, H2-M3 (HLA-G), CD47, CD200, and Mfge8.

In some embodiments, the engineered islets includes one or more modifications, such as genomic modifications, that reduce expression of one or more MHC class I molecules and a modification that increases expression of CD47. In other words, the engineered islets comprises exogenous CD47 proteins and exhibit reduced or silenced surface expression of one or more one or more MHC class I molecules. In some embodiments, the engineered islets includes one or more genomic modifications that reduce expression of one or more MHC class II molecules and a modification that increases expression of CD47. In some instances, the engineered islets comprises exogenous CD47 nucleic acids and proteins and exhibit reduced or silenced surface expression of one or more MHC class I molecules. In some embodiments, the engineered islets includes one or more genomic modifications that reduce or eliminate expression of one or more MHC class II molecules, one or more genomic modifications that reduce or eliminate expression of one or more MHC class II molecules, and a modification that increases expression of CD47. In some embodiments, the engineered islets comprises exogenous CD47 proteins, exhibit reduced or silenced surface expression of one or more MHC class I molecules and exhibit reduced or lack surface expression of one or more MHC class II molecules. In many embodiments, the engineered islets is a B2M^{indel/indel}, CIITA^{indel/indel}, CD47tg cell.

In some embodiments, the engineered islets elicits a reduced level of immune activation or no immune activation upon administration to a recipient subject. In some embodiments, the engineered islets elicits a reduced level of systemic TH1 activation or no systemic TH1 activation in a recipient subject. In some embodiments, the engineered islets elicits a reduced level of immune activation of peripheral blood mononuclear cells (PBMCs) or no immune activation of PBMCs in a recipient subject. In some embodiments, the engineered islets elicits a reduced level of donor-specific IgG antibodies or no donor specific IgG antibodies against the cells upon administration to a recipient subject. In some embodiments, the engineered islets elicits a reduced level of IgM and IgG antibody production or no IgM and IgG antibody production against the cells in a recipient subject. In some embodiments, the engineered islets elicits a reduced level of cytotoxic T cell killing of the cells upon administration to a recipient subject.

In some embodiments, the engineered islets provided herein comprises a selection agent. In some embodiments, the selection agent is a kill switch. A kill switch can be incorporated to function as a “safety switch” that can cause the death of the engineered islets, such as after the engineered islets is administered to a subject and if the engineered islets should grow and divide in an undesired manner. The result is specifically eliminating cells expressing a specific gene. In some embodiments, the kill switch is the herpesvirus thymidine kinase (HSV-tk) gene and the trigger is ganciclovir. In other embodiments, the kill switch is the Escherichia coli cytosine deaminase (EC-CD) gene and the trigger is 5-fluorocytosine (5-FC) (Barese et al, Mol. Therap. 20(10): 1932-1943 (2012), Xu et al, Cell Res. 8:73-8 (1998), both incorporated herein by reference in their entirety).

In other embodiments, the kill switch is an inducible Caspase protein. An inducible Caspase protein comprises at least a portion of a Caspase protein capable of inducing apoptosis. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, API 903. Thus, the elimination function of iCasp9 in the instant invention is triggered by the administration of a chemical inducer of dimerization (CID). In some embodiments, the CID is the small molecule drug API 903. Dimerization causes the rapid induction of apoptosis. (See WO2011146862; Stasi et al, N. Engl. J. Med 365; 18 (2011); Tey et al, Biol. Blood Marrow Transplant. 13:913-924 (2007), each of which are incorporated by reference herein in their entirety.)

Inclusion of a kill switch allows for controlled killing of the cells in the event of cytotoxicity or other negative consequences to the recipient, thus increasing the safety of cell-based therapies, including those using tolerogenic factors.

In some embodiments, a kill switch can be incorporated into, such as introduced, into the engineered islets provided herein to provide the ability to induce death or apoptosis of the engineered islets containing the kill switch, for example if the cells grow and divide in an undesired manner or cause excessive toxicity to the host. Thus, the use of kill switches enables one to conditionally eliminate aberrant cells in vivo and can be a critical step for the application of cell therapies in the clinic. Kill switches and their uses thereof are described in, for example, Duzgune§, Origins of Suicide Gene Therapy (2019); Duzgune§ (eds), Suicide Gene Therapy. Methods in Molecular Biology, vol. 1895 (Humana Press, New York, NY) (for HSV-tk, cytosine deaminase, nitroreductase, purine nucleoside phosphorylase, and horseradish peroxidase); Zhou and Brenner, Exp Hematol 44(11):1013-1019 (2016) (for iCaspase9); Wang et al., Blood 18(5):1255-1263 (2001) (for huEGFR); U.S. Patent Application Publication No. 20180002397 (for HER1); and Philip et al., Blood 124(8):1277-1287 (2014) (for RQR8).

In some embodiments, the kill switch can cause cell death in a controlled manner, for example, in the presence of a drug or prodrug or upon activation by a selective exogenous compound. In some embodiments, the kill switch is selected from the group consisting of herpes simplex virus thymidine kinase (HSV-tk), cytosine deaminase (CyD), nitroreductase (NTR), purine nucleoside phosphorylase (PNP), horseradish peroxidase, inducible caspase 9 (iCasp9), rapamycin-activated caspase 9 (rapaCasp9), chemically regulated-SH2-delivered inhibitory tail (CRASH-IT), CCR4, CD16, CD19, CD20, CD30, EGFR, GD2, HER1, HER2, MUC1, PSMA, and RQR8.

In some embodiments, the kill switch may be a transgene encoding a product with cell killing capabilities when activated by a drug or prodrug, for example, by turning a non-toxic prodrug to a toxic metabolite inside the cell. In these embodiments, cell killing is activated by contacting a engineered islets with the drug or prodrug. In some cases, the kill switch is HSV-tk, which converts ganciclovir (GCV) to GCV-triphosphate, thereby interfering with DNA synthesis and killing dividing cells. In some cases, the kill switch is CyD or a variant thereof, which converts the antifungal drug 5-fluorocytosine (5-FC) to cytotoxic 5-fluorouracil (5-FU) by catalyzing the hydrolytic deamination of cytosine into uracil. 5-FU is further converted to potent anti-metabolites (5-FdUMP, 5-FdUTP, 5-FUTP) by cellular enzymes. These compounds inhibit thymidylate synthase and the production of RNA and DNA, resulting in cell death. In some cases, the kill switch is NTR or a variant thereof, which can act on the prodrug CB 1954 via reduction of the nitro groups to reactive N-hydroxylamine intermediates that are toxic in proliferating and nonproliferating cells. In some cases, the kill switch is PNP or a variant thereof, which can turn prodrug 6-methylpurine deoxyriboside or fludarabine into toxic metabolites to both proliferating and nonproliferating cells. In some cases, the kill switch is horseradish peroxidase or a variant thereof, which can catalyze indole-3-acetic acid (IAA) to a potent cytotoxin and thus achieve cell killing.

In some embodiments, the kill switch may be an iCasp9. Caspase 9 is a component of the intrinsic mitochondrial apoptotic pathway which, under physiological conditions, is activated by the release of cytochrome C from damaged mitochondria. Activated caspase 9 then activates caspase 3, which triggers terminal effector molecules leading to apoptosis. The iCasp9 may be generated by fusing a truncated caspase 9 (without its physiological dimerization domain or caspase activation domain) to a FK506 binding protein (FKBP), FKBP12-F36V, via a peptide linker. The iCasp9 has low dimer-independent basal activity and can be stably expressed in host cells (e.g., human T cells) without impairing their phenotype, function, or antigen specificity. However, in the presence of chemical inducer of dimerization (CID), such as rimiducid (AP1903), AP20187, and rapamycin, iCasp9 can undergo inducible dimerization and activate the downstream caspase molecules, resulting in apoptosis of cells expressing the iCasp9. See, e.g., PCT Application Publication No. WO2011/146862; Stasi et al., N. Engl. J. Med. 365; 18 (2011); Tey et al., Biol. Blood Marrow Transplant 13:913-924 (2007). In particular, the rapamycininducible caspase 9 variant is called rapaCasp9. See Stavrou et al., Mal. Ther. 26(5):1266-1276 (2018). Thus, iCasp9 can be used as a kill switch to achieve controlled killing of the host cells.

In some embodiments, the kill switch may be a membrane-expressed protein which allows for cell depletion after administration of a specific antibody to that protein. kill switches of this category may include, for example, one or more transgene encoding CCR4, CD16, CD19, CD20, CD30, EGFR, GD2, HER1, HER2, MUC1, PSMA, or RQR8 for surface expression thereof. These proteins may have surface epitopes that can be targeted by specific antibodies. In some embodiments, the kill switch comprises CCR4, which can be recognized by an anti-CCR4 antibody. Non-limiting examples of suitable anti-CCR4 antibodies include mogamulizumab and biosimilars thereof. In some embodiments, the kill switch comprises CD16 or CD30, which can be recognized by an anti-CD16 or anti-CD30 antibody. Non-limiting examples of such anti-CD16 or anti-CD30 antibody include AFM13 and biosimilars thereof. In some embodiments, the kill switch comprises CD19, which can be recognized by an anti-CD19 antibody. Non-limiting examples of such anti-CD19 antibody include MOR208 and biosimilars thereof. In some embodiments, the kill switch comprises CD20, which can be recognized by an anti-CD20 antibody. Non-limiting examples of such anti-CD20 antibody include obinutuzumab, ublituximab, ocaratuzumab, rituximab, rituximab-R11b, and biosimilars thereof. Cells that express the kill switch are thus CD20-positive and can be targeted for killing through administration of an anti-CD20 antibody as described. In some embodiments, the kill switch comprises EGFR, which can be recognized by an anti-EGFR antibody. Non-limiting examples of such anti-EGFR antibody include tomuzotuximab, R05083945 (GA201), cetuximab, and biosimilars thereof. In some embodiments, the kill switch comprises GD2, which can be recognized by an anti-GD2 antibody. Non-limiting examples of such anti-GD2 antibody include Hu14.18K322A, Hu14.18-IL2, Hu3F8, dinituximab, c.60C3-R11c, and biosimilars thereof.

In some embodiments, the kill switch may be an exogenously administered agent that recognizes one or more tolerogenic factor on the surface of the engineered islets. In some embodiments, the exogenously administered agent is an antibody directed against or specific to a tolerogenic agent, e.g., an anti-CD47 antibody. By recognizing and blocking a tolerogenic factor on engineered islets, an exogenously administered antibody may block the immune inhibitory functions of the tolerogenic factor thereby re-sensitizing the immune system to the engineered islets. For instance, for a engineered islets that overexpresses CD47 an exogenously administered anti-CD47 antibody may be administered to the subject, resulting in masking of CD47 on the engineered islets and triggering of an immune response to the engineered islets.

In some embodiments, the method further comprises introducing an expression vector comprising an inducible kill into the cell.

In some embodiments, the tolerogenic factor is CD47 and the cell includes an exogenous polynucleotide encoding a CD47 protein. In some embodiments, the cell expresses an exogenous CD47 polypeptide.

In some embodiments, a method disclosed herein comprises administering to a subject in need thereof a CD47-SIRPα blockade agent, wherein the subject was previously administered a engineered islets engineered to express an exogenous CD47 polypeptide. In some embodiments, the CD47-SIRPα blockade agent comprises a CD47-binding domain. In some embodiments, the CD47-binding domain comprises signal regulatory protein alpha (SIRPα) or a fragment thereof. In some embodiments, the CD47-SIRPα blockade agent comprises an immunoglobulin G (IgG) Fc domain. In some embodiments, the IgG Fc domain comprises an IgG1 Fc domain. In some embodiments, the IgG1 Fc domain comprises a fragment of a human antibody. In some embodiments, the CD47-SIRPα blockade agent is selected from the group consisting of TTI-621, TTI-622, and ALX148. In some embodiments, the CD47-SIRPα blockade agent is TTI-621, TTI-622, and ALX148. In some embodiments, the CD47-SIRPα blockade agent is TTI-622. In some embodiments, the CD47-SIRPα blockade agent is ALX148. In some embodiments, the IgG Fc domain comprises an IgG4 Fc domain. In some embodiments, the CD47-SIRPα blockade agent is an antibody. In some embodiments, the antibody is selected from the group consisting of MIAP410, B6H12, and Magrolimab. In some embodiments, the antibody is MIAP410. In some embodiments, the antibody is B6H12. In some embodiments, the antibody is Magrolimab. In some embodiments, the antibody is selected from the group consisting of AO-176, IBI188 (letaplimab), STI-6643, and ZL-1201. In some embodiments, the antibody is AO-176 (Arch). In some embodiments, the antibody is IBI188 (letaplimab) (Innovent). In some embodiments, the antibody is STI-6643 (Sorrento). In some embodiments, the antibody is ZL-1201 (Zai).

In some embodiments, useful antibodies or fragments thereof that bind CD47 can be selected from a group that includes magrolimab ((Hu5F9-G4)) (Forty Seven, Inc.; Gilead Sciences, Inc.), urabrelimab, CC-90002 (Celgene; Bristol-Myers Squibb), IBI-188 (Innovent Biologics), IBI-322 (Innovent Biologics), TG-1801 (TG Therapeutics; also known as NI-1701, Novimmune SA), ALX148 (ALX Oncology), TJ011133 (also known as TJC4, I-Mab Biopharma), FA3M3, ZL-1201 (Zai Lab Co., Ltd), AK117 (Akesbio Australia Pty, Ltd.), AO-176 (Arch Oncology), SRF231 (Surface Oncology), GenSci-059 (GeneScience), C47B157 (Janssen Research and Development), C47B161 (Janssen Research and Development), C47B167 (Janssen Research and Development), C47B222 (Janssen Research and Development), C47B227 (Janssen Research and Development), Vx-1004 (Corvus Pharmaceuticals), HMBD004 (Hummingbird Bioscience Pte Ltd), SHR-1603 (Hengrui), AMMS4-G4 (Beijing Institute of Biotechnology), RTX-CD47 (University of Groningen), and IMC-002. (Samsung Biologics; ImmuneOncia Therapeutics). In some embodiments, the antibody or fragment thereof does not compete for CD47 binding with an antibody selected from a group that includes magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof competes for CD47 binding with an antibody selected from magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof that binds CD47 is selected from a group that includes a single-chain Fv fragment (scFv) against CD47, a Fab against CD47, a VHH nanobody against CD47, a DARPin against CD47, and variants thereof. In some embodiments, the scFv against CD47, a Fab against CD47, and variants thereof are based on the antigen binding domains of any of the antibodies selected from a group that includes magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002.

In some embodiments, the CD47 antagonist provides CD47 blockade. Methods and agents for CD47 blockade are described in PCT/US2021/054326, which is incorporated by reference in its entirety.

In some embodiments, the engineered islets is derived from a source cell already comprising one or more of the desired modifications. In some embodiments, in view of the teachings provided herein one of ordinary skill in the art will readily appreciate how to assess what modifications are required to arrive at the desired final form of a engineered islets and that not all reduced or increased levels of target components are achieved via active engineering. In some embodiments, the modifications of the engineered islets may be in any order, and not necessarily the order listed in the descriptive language provided herein.

Once altered, the presence of expression of any of the molecule described herein can be assayed using known techniques, such as Western blots, ELISA assays, FACS assays, flow cytometry, and the like.

b. Exemplary Embodiments of Engineered Islets

In some embodiments, the engineered hypoimmunogenic islets comprise any of the vectors described herein (e.g., in section 11.6 above), wherein the vector generates germline modifications that: (a) inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules; and/or (b) increase expression of one or more tolerogenic factors, wherein the increased expression is relative to a control or wild-type islet that does not comprise the modifications. In some embodiments, the one or more molecules that regulate expression of the one or more MHC class I molecules is B2M. In some embodiments, the one or more molecules that regulate expression of the one or more MHC class II molecules is CIITA. In some embodiments, expression of HLA-A, HLA-B, HLA-C, HLA-DP, HLA-DQ, and HLA-DR are reduced in the engineered hypoimmunogenic islets. In some embodiments, the engineered hypoimmunogenic islet cell further comprises a modification to increase expression of an exogenous kill switch.

In some embodiments, the modifications comprise a modification that regulates the expression of the one or more MHC class I molecules, and the modification inactivates or disrupts one or more alleles of B2M. In some embodiments, the modification that inactivates or disrupts one or more alleles of B2M reduces mRNA expression of the B2M gene. In some embodiments, the modification that inactivates or disrupts one or more alleles of B2M reduces protein expression of B2M. In some embodiments, the modification that inactivates or disrupts one or more alleles of B2M comprises: i) inactivation or disruption of one allele of the B2M gene; ii) inactivation or disruption of both alleles of the B2M gene; or iii) inactivation or disruption of all B2M coding alleles in the cell. In some embodiments, the inactivation or disruption comprises an indel in the B2M gene. In some embodiments, the inactivation or disruption comprises a frameshift mutation or a deletion of a contiguous stretch of genomic DNA of the B2M gene.

In some embodiments, the one or more modifications comprise a modification that regulates cell surface protein expression of the one or more MHC class I molecules and the modification inactivates or disrupts one or more alleles of B2M. In some embodiments, the modification that regulates cell surface protein expression reduces cell surface trafficking of the one or more MHC class I molecules. In some embodiments, the one or more modifications reduce a function of the one or more MHC class I molecules, optionally wherein the function is antigen presentation.

In some embodiments, the modification is a modification that regulates expression of the one or more MHC class II molecules, and the modification inactivates or disrupts one or more alleles of CIITA. In some embodiments, the modification that inactivates or disrupts one or more alleles of CIITA reduces protein expression of CIITA. In some embodiments, the modification that inactivates or disrupts one or more alleles of CIITA comprises: i) inactivation or disruption of one allele of the CIITA gene; ii) inactivation or disruption of both alleles of the CIITA gene; or iii) inactivation or disruption of all CIITA coding alleles in the cell. In some embodiments, the inactivation or disruption comprises an indel in the CIITA gene. In some embodiments, the inactivation or disruption is a frameshift mutation or a deletion of a contiguous stretch of genomic DNA of the CIITA gene.

In some embodiments, the vector comprises one or more transgenes comprising one or more tolerogenic factors selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF. In some embodiments, at least one of the one or more tolerogenic factors is CD47. In some embodiments, at least one of the one or more tolerogenic factors is PD-L1. In some embodiments, at least one of the one or more tolerogenic factors is HLA-E. In some embodiments, at least one of the one or more tolerogenic factors is HLA-G.

In some embodiments, the engineered hypoimmunogenic islet cells further comprises a modification to increase expression of an exogenous kill switch. In some embodiments, the kill switch is a system wherein upon activation, cells downregulate expression of the one or more tolerogenic factors and/or upregulate expression of one or more immune signaling molecules, thereby marking the cell for elimination by the host immune system. In some embodiments, the one or more immune signaling molecules is selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAETIH/ULBP2, RAET1/ULBP1, RAET1L/ULBP6, RAET1N/ULBP3, and other ligands of NKG2D. In some embodiments, the suicide gene is selected from the group consisting of cytosine deaminase (CyD), herpesvirus thymidine kinase (HSV-Tk), an inducible caspase 9 (iCaspase9), rapamycin-activated caspase 9 (rapaCasp9), and chemically regulated-SH2-delivered inhibitory tail (CRASH-IT). In some embodiments, the engineered hypoimmunogenic islet cells have the genotype B2M^{indel/indel}; CIITA^{indel/indel}; CD47tg; safety switch transgene.

In some embodiments, the one or more tolerogenic factors comprises CD47 and the engineered hypoimmunogenic islet cells expresses CD47 at a first level that is greater than at or about 5-fold over a second level expressed by the control or wild-type islet cells, optionally wherein CD47 is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type islet cells. In some embodiments, the one or more tolerogenic factors comprises CD47 and CD47 is expressed by the engineered hypoimmunogenic islet cells at greater than at or about 20,000 molecules per cell, optionally, wherein CD47 is expressed by the engineered hypoimmunogenic islet cells at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

In some embodiments, the engineered islets exhibit increased expression of CD47 and reduced expression of one or more molecules of the MHC class I complex. In some embodiments, the engineered islets exhibit increased expression of CD47 and reduced expression of one or more molecules of the MHC class I and MHC class II complexes. In some embodiments, the engineered islets express one or more exogenous complement inhibitor polypeptides selected from CD46, CD59, CD55, and any combinations thereof.

In some embodiments, engineered islets exhibit increased expression of CD47 and reduced expression of B2M. In some embodiments, engineered islets exhibit increased expression of CD47 and reduced expression of CIITA. In some embodiments, the engineered islets exhibit increased expression of CD47 and reduced expression of NLRC5. In some embodiments, the engineered islets exhibit increased expression of CD47 and reduced expression of one or more molecules of B2M and CIITA. In some embodiments, the engineered islets exhibit increased expression of CD47 and reduced expression of one or more molecules of B2M and NLRC5. In some embodiments, the engineered islets exhibit increased expression of CD47 and reduced expression of one or more molecules of B2M, CIITA and NLRC5. Any of the engineered islets described herein can also exhibit increased expression of one or more factors selected from the group including, but not limited to, DUX4, CD24, CD27, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, and Serpinb9. In some embodiments, the engineered islets express one or more exogenous complement inhibitor polypeptides selected from CD46, CD59, CD55, and any combinations thereof.

In some embodiments, the engineered islets exhibit increased expression of CD47 and at least one complement inhibitor selected from the group consisting of CD46, CD59, CD55, and any combination thereof, and reduced expression of one or more molecules of the MHC class I complex. In some embodiments, the engineered islets exhibit increased expression of CD47 and at least one complement inhibitor selected from the group consisting of CD46, CD59, CD55, and any combination thereof, and reduced expression of one or more molecules of the MHC class II complex. In some embodiments, the engineered islets exhibit increased expression of CD47 and at least one complement inhibitor selected from the group consisting of CD46, CD59, CD55, and any combination thereof, and reduced expression of one or more molecules of the MHC class II and MHC class II complexes. In some embodiments, the engineered islets and populations thereof exhibit increased expression of CD47 and at least one complement inhibitor selected from the group consisting of CD46, CD59, CD55, and any combination thereof, and reduced expression of B2M. In some embodiments, the engineered islets and populations thereof exhibit increased expression of CD47 and at least one complement inhibitor selected from the group consisting of CD46, CD59, CD55, and any combination thereof, and reduced expression of CIITA. In some embodiments, the engineered islets and populations thereof exhibit increased expression of CD47 and at least one complement inhibitor selected from the group consisting of CD46, CD59, CD55, and any combination thereof, and reduced expression of NLRC5. In some embodiments, the engineered islets and populations thereof exhibit increased expression of CD47 and at least one complement inhibitor selected from the group consisting of CD46, CD59, CD55, and any combination thereof, and reduced expression of one or more molecules of B2M and CIITA. In some embodiments, the engineered islets and populations thereof exhibit increased expression of CD47 and at least one complement inhibitor selected from the group consisting of CD46, CD59, CD55, and any combination thereof, and reduced expression of one or more molecules of B2M and NLRC5. In some embodiments, the engineered islets and populations thereof exhibit increased expression of CD47 and at least one complement inhibitor selected from the group consisting of CD46, CD59, CD55, and any combination thereof, and reduced expression of one or more molecules of CIITA and NLRC5. In some embodiments, the engineered islets and populations thereof exhibit increased expression of CD47 at least one complement inhibitor selected from the group consisting of CD46, CD59, CD55, and any combination thereof, and reduced expression of one or more molecules of B2M, CIITA and NLRC5.

In some embodiments, an engineered islets exhibits increased or decreased expression of the one more target molecules (e.g. MHC class I or class II, or CD47) in which the increase or decrease in expression is retained or similar (e.g. 75% to 100% of the level) compared to the unmodified or wild-type cell. Also provided herein is a population of engineered islets that include a plurality of cells that exhibit increased or decreased expression of the one more target molecules (e.g. MHC class I or class II, or CD47).

In some embodiments, at least at or about 50%, at least at or about 60%, at least at or about 70%, at least at or about 80%, or at least at or about 90% of the cells in the population are eliminated for expression of MHC class I or for B2M. In some embodiments, at least at or about 50%, at least at or about 60%, at least at or about 70%, at least at or about 80%, or at least at or about 90% of the cells in the population are eliminated for expression of MHC class II or for CIITA.

In some embodiments, least at or about 50%, at least at or about 60%, at least at or about 70%, at least at or about 80%, or at least at or about 90% of the cells in the population level have increased expression of the tolerogenic factor (CD47) that is greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a wild-type primary beta cell or an unmodified pluripotent stem cell or an unmodified SC-beta differentiated from the unmodified pluripotent stem cell. In some embodiments, at least at or about 50%, at least at or about 60%, at least at or about 70%, at least at or about 80%, or at least at or about 90% of the cells in the population expresses the tolerogenic factor (e.g. CD47) at greater than at or about 20,000 molecules per cell, at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

One skilled in the art will appreciate that levels of expression such as increased or reduced expression of a gene, protein or molecule can be referenced or compared to a comparable cell. In some embodiments, a modified cell (e.g., an engineered islet, such as a modified beta islet cell) having increased expression of a protein (e.g., CD46, CD59. CD55, CD47, or any other tolerogenic factor) refers to a modified cell having a higher level of the protein compared to an unmodified cell. In some embodiments, an engineered islets having increased expression of a protein (e.g., CD46, CD59. CD55, CD47, or any other tolerogenic factor) is a cell comprising modifications, wherein the cell comprising modifications has a higher level of the protein compared to a cell without said modifications (e.g., beta cell without the modifications may comprise other modifications). In some embodiments, an engineered islet having reduced expression of a protein (e.g., B2M, or CIITA) is a cell comprising modifications, wherein the cell comprising modifications has a lower level of the protein or RNA compared to a cell without said modifications (e.g., beta cell without the modifications may comprise other modifications). In some embodiments, the engineered islets express one or more exogenous complement inhibitor polypeptides selected from CD46, CD59, CD55, and any combinations thereof.

In one embodiment, provided herein are engineered islets expressing exogenous CD47 polypeptides and having reduced expression of either one or more MHC class I complex proteins, one or more MHC class II complex proteins, or any combination of MHC class I and class II complex proteins. In another embodiment, the engineered islets express exogenous CD47 polypeptides and express reduced levels of B2M and CIITA polypeptides. In some embodiments, the engineered islets express exogenous CD47 polypeptides and possess modifications of the B2M and CIITA genes. In some instances, the modifications inactivate the B2M and CIITA genes. In some embodiments, the modified cells express one or more exogenous complement inhibitor polypeptides selected from CD46, CD59, CD55, and any combinations thereof.

c. Exemplary Features of SC-β cells

The provided modified SC-beta cells, including those obtained by in vitro differentiation from pluripotent stem cells such as modified pluripotent stem cells, are highly functional SC-β cells. The modified SC-beta cell or population exhibits a GSIS response both in vitro and in vivo. The isolated SC-beta cell or population also exhibits at least one characteristic feature of a mature endogenous beta cell. In some aspects, a modified SC-beta cell or population thereof exhibits a stimulation index of between about 1.4 and about 2.4. In some aspects, a modified SC-beta cell or population thereof produces insulin at between approximately 300 uIU to about 4000 uIU per 30 minute per 106 total cells incubation at a high glucose concentration.

In certain embodiments, static insulin secretion is greater than about 1 uIU/103 cells/hour at high glucose (e.g., 20 mM). In certain embodiments, the static insulin secretion is greater than about 1.5 uIU/103 cells/hour at high glucose. In certain embodiments, the static insulin secretion is greater than about 2.0 uIU/103 cells/hour at high glucose. In certain embodiments, the static insulin secretion is greater than about 2.5 uIU/103 cells/hour at high glucose. In certain embodiments, the static insulin secretion is greater than about 3.0 uIU/103 cells/hour at high glucose. In certain embodiments, the static insulin secretion is greater than about 3.5 uIU/103 cells/hour at high glucose. In certain embodiments, the static insulin secretion is greater than about 4.0 uIU/103 cells/hour at high glucose. In certain embodiments, the static stimulation index is defined as a ratio of 20 mM glucose to 2 mM glucose, incubated for 1 hr.

Assays to assess functional activity of the SC-b cells include static and dynamic glucose stimulated insulin secretion (GSIS) assays to measure glucose responsiveness, insulin content and proinsulin-to-insulin ratio to determine intracellular insulin levels and processing, flow cytometry to measure the percentages of the different hormone-producing cells, and qRT-PCR and immunostaining to confirm the presence of β-cell-specific markers. In particular, SC-β cells can be identified by their coexpression of C-peptide and NKX6-1, while chromogranin A (CHGA) marks the general endocrine population. After aggregation, >80% of the cells in these clusters should express CHGA, with ˜20-60% of these cells being C-peptide+/NKX6-1+.

In certain embodiments, the SC-β cells achieve both first and second-phase dynamic insulin secretion. In some embodiments, the SC-β cells achieve equivalent functional capabilities of human islets. In certain embodiments, the SC-β cells retain functionality for 1 or more days. In certain embodiments, the SC-pp cells retain functionality for more than 1 week. In certain embodiments, the SC-β cells may be used to treat or reverse severe preexisting diabetes at a rate similar to primary human islets, outperforming cells generated with a suspension-based protocol. In certain embodiments, the SC-β cells, when transplanted, are capable of maintaining normoglycemia indefinitely.

### Hepatocyte Cells

In some embodiments, the engineered cells or populations of cells that are provided herein are hepatocytes, such as primary hepatocytes or hepatocytes differentiated from pluripotent cells, stem cells or iPSCs. In some embodiments, primary hepatocytes are isolated or obtained from one or more individual donor subjects, such as one or more individual healthy donor (e.g., a subject that is not known or suspected of, e.g., not exhibiting clinical signs of, a disease or infection). As will be appreciated by those in the art, methods of isolating or obtaining hepatocytes from an individual can be achieved using known techniques.

In some embodiments, primary hepatocytes are obtained (e.g., harvested, extracted, removed, or taken) from a subject or an individual. In some embodiments, primary hepatocytes are produced from a pool of hepatocytes such that the hepatocytes are from one or more subjects (e.g., one or more human including one or more healthy humans). In some embodiments, the pool of primary hepatocytes is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects.

In some embodiments, the cells as provided herein are engineered hepatocytes differentiated from engineered iPSCs that contain any of the vectors described herein and that are differentiated into hepatocytes. As will be appreciated by those skilled in the art, the methods for differentiation depend on the desired cell type using known techniques.

In some embodiments, engineered pluripotent cells containing any of the vectors described herein are differentiated into hepatocytes. There are a number of techniques that can be used to differentiate engineered pluripotent cells into hepatocytes; see for example, Pettinato et al., doi: 10.1038/spre32888; Snykers et al., Methods Mol Biol, 2011 698:305-314; Si-Tayeb et al., Hepatology, 2010, 51:297-305; and Asgari et al., Stem Cell Rev, 2013, 9(4):493-504, all of which are incorporated herein by reference in their entirety. Differentiation can be assayed as is known in the art, generally by evaluating the presence of hepatocyte-associated and/or specific markers, including, but not limited to, albumin, alpha fetoprotein, and fibrinogen. Differentiation can also be measured functionally, such as the metabolization of ammonia, LDL storage and uptake, ICG uptake and release, and glycogen storage.

In some embodiments, the engineered hepatocytes, such as primary hepatocytes isolated from one or more individual donors (e.g., healthy donors) or hepatocytes differentiated from iPSCs derived from one or more individual donors (e.g., healthy donors), are maintained in culture and in some cases expanded and/or cryopreserved. In certain embodiments, the population of hepatocytes are cryopreserved.

In some embodiments, the present disclosure is directed to engineered hepatocytes, such as primary hepatocytes isolated from one or more individual donors (e.g., healthy donors) or hepatocytes differentiated from iPSCs derived from one or more individual donors (e.g., healthy donors).

In some embodiments, the provided engineered hepatocytes evade immune recognition. In some embodiments, the engineered hepatocytes described herein, such as primary hepatocytes isolated from one or more individual donors (e.g., healthy donors) or hepatocytes differentiated from iPSCs derived from one or more individual donors (e.g., healthy donors), do not activate an immune response.

### T Cells

In some embodiments, the engineered cells or populations of cells provided herein are T lymphocytes (also called T cells), such as primary T lymphocytes or T cells. In some embodiments, primary T lymphocytes are isolated or obtained from one or more individual donor subjects, such as one or more individual healthy donor (e.g., a subject that is not known or suspected of, e.g., not exhibiting clinical signs of, a disease or infection). In some instances, the T cells are populations or subpopulations of primary T cells from one or more individuals. As will be appreciated by those skilled in the art, methods of isolating or obtaining T lymphocytes from an individual can be achieved using known techniques.

In some embodiments, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, the pool of primary T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects.

In some embodiments, the engineered cells or populations of cells as provided herein are T lymphocytes differentiated from engineered pluripotent cells that contain any of the vectors described herein and that are differentiated into T lymphocytes. As will be appreciated by those skilled in the art, the methods for differentiation depend on the desired cell type using known techniques.

Methods for generating T cells from pluripotent stem cells (e.g., iPSC) are described, for example, in Iriguchi et al., Nature Communications 12, 430 (2021); Themeli et al., Nature Biotechnology 31:928-933 (2013).

Non-limiting examples of T cells, such as primary T cells, include CD3+ T cells, CD4+ T cells, CD8+ T cells, naive T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), 76 T cells, and any other subtype of T cells. In some embodiments, the primary T cells are selected from the group consisting of: cytotoxic T cells, helper T cells, memory T cells, regulatory T cells, tumor infiltrating lymphocytes, and any combination thereof.

Exemplary T cells that may be used according to the present disclosure include, without limitation, cytotoxic T cells, helper T cells, memory T cells, central memory T cells, effector memory T cells, effector memory RA T cells, regulatory T cells, tissue infiltrating lymphocytes, and combinations thereof. In many embodiments, the T cells express CCR7, CD27, CD28, and CD45RA. In some embodiments, the central T cells express CCR7, CD27, CD28, and CD45RO. In other embodiments, the effector memory T cells express PD-1, CD27, CD28, and CD45RO. In other embodiments, the effector memory RA T cells express PD-1, CD57, and CD45RA.

In some embodiments, the present disclosure is directed to engineered T cells, such as primary T cells isolated from one or more individual donors (e.g., healthy donors) or T cells differentiated from iPSCs derived from one or more individual donors (e.g., healthy donors).

In some embodiments, the provided engineered T cells evade immune recognition. In some embodiments, the engineered T cells described herein, such as primary T cells isolated from one or more individual donors (e.g., healthy donors) or T cells differentiated from iPSCs derived from one or more individual donors (e.g., healthy donors), do not activate an immune response.

### Natural Killer Cells

In some embodiments, the engineered cells or populations of cells that are provided herein are Natural Killer (NK) cells. In some embodiments, the NK cells are isolated or obtained from one or more individual donor subjects, such as one or more individual healthy donor (e.g., a subject that is not known or suspected of, e.g., not exhibiting clinical signs of, a disease or infection). In some instances, the NK cells are populations or subpopulations of NK cells from one or more individuals. As will be appreciated by those skilled in the art, methods of isolating or obtaining NK cells from an individual can be achieved using known techniques.

In some embodiments, the engineered cells or populations of cells as provided herein are NK cells differentiated from engineered pluripotent cells that contain any of the vectors described herein and that are differentiated into NK cells. As will be appreciated by those skilled in the art, the methods for differentiation depend on the desired cell type using known techniques.

Methods for generating NK cells from pluripotent stem cells (e.g., iPSC) are described, for example, in U.S. patent Ser. No. 10/626,373; Shankar et al., Stem Cell Res Ther. 2020; 11: 234; Euchner et al., Frontiers in Immunology, 2021; 12, Article 640672, doi=10.3389/fimmu.2021.640672.

In some embodiments, NK cells are obtained (e.g., harvested, extracted, removed, or taken) from a subject or an individual. In some embodiments, NK cells are produced from a pool of NK cells such that the NK cells are from one or more subjects (e.g., one or more human including one or more healthy humans). In some embodiments, the pool of primary NK cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects.

In some embodiments, NK cells, including primary NK cells isolated from one or more individual donors (e.g., healthy donors) or NK cells differentiated from iPSCs derived from one or more individual donors (e.g., healthy donors) express CD56 (e.g., CD56dim or CD56bright) and lack CD3 (e.g., CD3neg). In some embodiments, NK cells as described herein may also express the low-affinity Fc receptor CD16, which mediate ADCC. In some embodiments, the NK cells also express one or more natural killer cell receptors NKG2A and NKG2D or one or more natural cytotoxicity receptors NKp46, NKp44, NKp30. For example, for the case of primary NK cells, in specific cases, the primary cells may be isolated from a starting source of NK cells, such as a sample containing peripheral blood mononuclear cells (PBMCs), by depletion of cells positive for CD3, CD14, and/or CD19. For instance, the cells may be subject to depletion using immunomagnetic beads having attached thereto antibodies to CD3, CD14, and/or CD19, respectively, thereby producing an enriched population of NK cells. In other cases, primary NK cells may be isolated from a starting source that is a mixed population (e.g., PBMCs) by selecting cells for the presence of one or more markers on the NK cells, such as CD56, CD16, NKp46, and/or NKG2D.

In some embodiments, prior to the engineering as described herein, the NK cells, such as isolated primary NK cells, may be subject to one or more expansion or activation step. In some embodiments, expansion may be achieved by culturing of the NK cells with feeder cells, such as antigen presenting cells that may or may not be irradiated. The ratio of NK cells to antigen presenting cells (APCs) in the expansion step may be of a certain number, such as 1:1, 1:1.5, 1:2, or 1:3, for example. In certain aspects, the APCs are engineered to express membrane-bound IL-21 (mblL-21). In particular aspects, the APCs are alternatively or additionally engineered to express IL-21, IL-15, and/or IL-2. In particular embodiments, the media in which the expansion step(s) occurs comprises one or more agents to facilitate expansion, such as one or more recombinant cytokines. In specific embodiments, the media comprises one or more recombinant cytokines from IL-2, IL-15, IL-18, and/or IL-21. In some embodiments, the steps for engineering the NK cells by introducing the vectors as described herein is carried out 2-12 days after initiation of the expansion, such as on or about day 2, 3, 4, 5, 6, 7, 8, 9, 10, 11 or 12.

In some embodiments, the present technology is directed to engineered NK cells, such as primary NK cells isolated from one or more individual donors (e.g., healthy donors) or NK cells differentiated from iPSCs derived from one or more individual donors (e.g., healthy donors).

In some embodiments, the provided engineered NK cells evade immune recognition. In some embodiments, the engineered NK cells described herein, such as primary NK cells isolated from one or more individual donors (e.g., healthy donors), do not activate an immune response.

### Endothelial Cells

In some embodiments, the engineered cells or populations of cells that are provided herein are endothelial cells, such as primary endothelial cells. In some embodiments, primary endothelial cells are isolated or obtained from one or more individual donor subjects, such as one or more individual healthy donor (e.g., a subject that is not known or suspected of, e.g., not exhibiting clinical signs of, a disease or infection). As will be appreciated by those skilled in the art, methods of isolating or obtaining endothelial cells from an individual can be achieved using known techniques.

In some embodiments, primary endothelial cells are obtained (e.g., harvested, extracted, removed, or taken) from a subject or an individual. In some embodiments, primary endothelial cells are produced from a pool of endothelial cells such that the endothelial cells are from one or more subjects (e.g., one or more human including one or more healthy humans). In some embodiments, the pool of primary endothelial cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects.

In some embodiments, the engineered cells or populations of cells as provided herein are engineered endothelial cells differentiated from engineered iPSCs that contain any of the vectors described herein and that are differentiated into an endothelial cell type. As will be appreciated by those skilled in the art, the methods for differentiation depend on the desired cell type using known techniques.

In some embodiments, the engineered pluripotent cells described herein are differentiated into endothelial colony forming cells (ECFCs) to form new blood vessels. Techniques to differentiate endothelial cells are known. See, e.g., Prasain et al., doi: 10.1038/nbt.3048, incorporated herein by reference in its entirety. Differentiation can be assayed as is known in the art, generally by evaluating the presence of endothelial cell associated or specific markers or by measuring functionality.

In some embodiments, the method of producing engineered endothelial cells from engineered pluripotent cells by in vitro differentiation comprises: (a) culturing a population of engineered iPSCs in a first culture medium comprising a GSK inhibitor; (b) culturing the population of engineered iPSCs cells in a second culture medium comprising VEGF and bFGF to produce a population of pre-endothelial cells; and (c) culturing the population of pre-endothelial cells in a third culture medium comprising a ROCK inhibitor and an ALK inhibitor to produce a population of differentiated endothelial cells that are engineered to contain the one or more exogenous sequences as described herein.

In some embodiments, the GSK inhibitor is CHIR-99021, a derivative thereof, or a variant thereof. In some instances, the GSK inhibitor is at a concentration ranging from about 1 mM to about 10 mM. In some embodiments, the ROCK inhibitor is Y-27632, a derivative thereof, or a variant thereof. In some instances, the ROCK inhibitor is at a concentration ranging from about 1 μM to about 20 μM. In some embodiments, the ALK inhibitor is SB-431542, a derivative thereof, or a variant thereof. In some instances, the ALK inhibitor is at a concentration ranging from about 0.5 μM to about 10 μM.

In some embodiments, the first culture medium comprises from 2 μM to about 10 μM of CHIR-99021. In some embodiments, the second culture medium comprises 50 ng/ml VEGF and 10 ng/ml bFGF. In other embodiments, the second culture medium further comprises Y-27632 and SB-431542. In various embodiments, the third culture medium comprises 10 μM Y-27632 and 1 pM SB-431542. In certain embodiments, the third culture medium further comprises VEGF and bFGF. In particular instances, the first culture medium and/or the second medium is absent of insulin.

The engineered cells or populations of cells provided herein can be cultured on a surface, such as a synthetic surface to support and/or promote differentiation of pluripotent cells into engineered endothelial cells. In some embodiments, the surface comprises a polymer material including, but not limited to, a homopolymer or copolymer of selected one or more acrylate monomers. Non-limiting examples of acrylate monomers and methacrylate monomers include tetra(ethylene glycol) diacrylate, glycerol dimethacrylate, 1,4-butanediol dimethacrylate, poly(ethylene glycol) diacrylate, di(ethylene glycol) dimethacrylate, tetra(ethyiene glycol) dimethacrylate, 1,6-hexanediol propoxylate diacrylate, neopentyl glycol diacrylate, trimethylolpropane benzoate diacrylate, trimethylolpropane eihoxylate (1 EO/QH) methyl, tricyclo[5.2.1.02,6]decane dimethanol diacrylate, neopentyl glycol exhoxylate diacrylate, and trimethylolpropane triacrylate. Acrylate synthesized as known in the art or obtained from a commercial vendor, such as Polysciences, Inc., Sigma Aldrich, Inc. and Sartomer, Inc.

In some embodiments, the endothelial cells may be seeded onto a polymer matrix. In some cases, the polymer matrix is biodegradable. Suitable biodegradable matrices are well known in the art and include collagen-GAG, collagen, fibrin, PLA, PGA, and PLA/PGA co-polymers. Additional biodegradable materials include poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes and polysaccharides.

Non-biodegradable polymers may also be used as well. Other non-biodegradable, yet biocompatible polymers include polypyrrole, polyanibnes, polythiophene, polystyrene, polyesters, non-biodegradable polyurethanes, polyureas, poly(ethylene vinyl acetate), polypropylene, polymethacrylate, polyethylene, polycarbonates, and poly(ethylene oxide). The polymer matrix may be formed in any shape, for example, as particles, a sponge, a tube, a sphere, a strand, a coiled strand, a capillary network, a film, a fiber, a mesh, or a sheet. The polymer matrix can be modified to include natural or synthetic extracellular matrix materials and factors.

The polymeric material can be dispersed on the surface of a support material. Useful support materials suitable for culturing cells include a ceramic substance, a glass, a plastic, a polymer or co-polymer, any combinations thereof, or a coating of one material on another. In some instances, a glass includes soda-lime glass, pyrex glass, vycor glass, quartz glass, silicon, or derivatives of these or the like.

In some instances, plastics or polymers including dendritic polymers include poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine or derivatives of these or the like. In some instances, copolymers include poly(vinyl acetate-co-maleic anhydride), poly(styrene-co-maleic anhydride), poly(ethylene-co-acrylic acid) or derivatives of these or the like.

Additional descriptions of endothelial cells and their differentiation for use in the methods provided herein are found in WO2020/018615, the disclosure of which is herein incorporated by reference in its entirety.

In some embodiments, the engineered endothelial cells, such as primary endothelial cells isolated from one or more individual donors (e.g., healthy donors) or endothelial cells differentiated from iPSCs derived from one or more individual donors (e.g., healthy donors), are maintained in culture and in some cases expanded and/or cryopreserved. In certain embodiments, the engineered endothelial cells are cryopreserved.

In some embodiments, the present disclosure is directed to engineered endothelial cells, such as primary endothelial cells isolated from one or more individual donors (e.g., healthy donors) or endothelial cells differentiated from iPSCs derived from one or more individual donors (e.g., healthy donors).

In some embodiments, the provided engineered endothelial cells evade immune recognition. In some embodiments, the engineered endothelial cells described herein, such as primary endothelial cells isolated from one or more individual donors (e.g., healthy donors) or endothelial cells differentiated from iPSCs derived from one or more individual donors (e.g., healthy donors), do not activate an immune response.

Exemplary endothelial cell types include, but are not limited to, a capillary endothelial cell, vascular endothelial cell, aortic endothelial cell, arterial endothelial cell, venous endothelial cell, renal endothelial cell, brain endothelial cell, liver endothelial cell, and the like.

The endothelial cells outlined herein, such as isolated primary endothelial cells or differentiated endothelial cells, can express one or more endothelial cell markers. Non-limiting examples of such markers include VE-cadherin (CD 144), ACE (angiotensin-converting enzyme) (CD 143), BNH9/BNF13, CD31, CD34, CD54 (ICAM-1), CD62E (E-Selectin), CD105 (Endoglin), CD146, Endocan (ESM-1), Endoglyx-1, Endomucin, Eotaxin-3, EPAS1 (Endothelial PAS domain protein 1), Factor VIII related antigen, FLI-1, Flk-1 (KDR, VEGFR-2), FLT-1 (VEGFR-1), GATA2, GBP-1 (guanylate-binding protein-1), GRO-alpha, HEX, ICAM-2 (intercellular adhesion molecule 2), LM02, LYVE-1, MRB (magic roundabout), Nucleolin, PAL-E (pathologische anatomie Leiden-endothelium), RTKs, sVCAM-1, TAL1, TEM1 (Tumor endothelial marker 1), TEM5 (Tumor endothelial marker 5), TEM7 (Tumor endothelial marker 7), thrombomodulin (TM, CD141), VCAM-1 (vascular cell adhesion molecule-1) (CD106), VEGF, vWF (von Willebrand factor), ZO-1, endothelial cell-selective adhesion molecule (ESAM), CD102, CD93, CD184, CD304, and DLL4.

### Epithelial Cells

In some embodiments, the engineered cells or populations of cells that are provided herein are retinal pigmented epithelium (RPE) cells, such as primary RPE cells. In some embodiments, the primary RPE cells are isolated or obtained from one or more individual donor subjects, such as one or more individual healthy donor (e.g., a subject that is not known or suspected of, e.g., not exhibiting clinical signs of, a disease or infection). As will be appreciated by those skilled in the art, methods of isolating or obtaining RPE cells from an individual can be achieved using known techniques.

In some embodiments, primary RPE cells are obtained (e.g., harvested, extracted, removed, or taken) from a subject or an individual. In some embodiments, primary RPE cells are produced from a pool of RPE cells such that the RPE cells are from one or more subjects (e.g., one or more human including one or more healthy humans). In some embodiments, the pool of primary RPE cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects.

In some embodiments, the cells as provided herein are engineered RPE cells differentiated from engineered iPSCs that contain any of the vectors described herein and that are differentiated into an engineered RPE cell. As will be appreciated by those skilled in the art, the methods for differentiation depend on the desired cell type using known techniques.

Useful methods for differentiating pluripotent stem cells into RPE cells are described in, for example, U.S. Pat. Nos. 9,458,428 and 9,850,463, the disclosures of which are herein incorporated by reference in their entirety. Additional methods for producing RPE cells from human induced pluripotent stem cells can be found in, for example, Lamba et al., PNAS, 2006, 103(34): 12769-12774; Mellough et al., Stem Cells, 2012, 30(4):673-686; Idelson et al., Cell Stem Cell, 2009, 5(4): 396-408; Rowland et al., Journal of Cellular Physiology, 2012, 227(2):457-466; Buchholz et al., Stem Cells Trans Med, 2013, 2(5): 384-393; and da Cruz et al., Nat Biotech, 2018, 36:328-337.

Human pluripotent stem cells have been differentiated into RPE cells using the techniques outlined in Kamao et al., Stem Cell Reports 2014:2:205-18, hereby incorporated by reference in its entirety; see also Mandai et al., N Engl J Med, 2017, 376:1038-1046, the contents herein incorporated in its entirety. Differentiation can be assayed as is known in the art, generally by evaluating the presence of RPE-associated and/or specific markers or by measuring functionality.

In some embodiments, the method of producing engineered retinal pigmented epithelium (RPE) cells from engineered pluripotent cells by in vitro differentiation comprises: (a) culturing the population of engineered pluripotent cells in a first culture medium comprising any one of the factors selected from activin A, bFGF, BMP4/7, DKK1, IGF1, noggin, a BMP inhibitor, an ALK inhibitor, a ROCK inhibitor, and a VEGFR inhibitor to produce a population of pre-RPE cells; and (b) culturing the pre-RPE cells in a second culture medium that is different than the first culture medium to produce engineered RPE cells. In some embodiments, the ALK inhibitor is SB-431542, a derivative thereof, or a variant thereof. In some instances, the ALK inhibitor is at a concentration ranging from about 2 mM to about 10 μM. In some embodiments, the ROCK inhibitor is Y-27632, a derivative thereof, or a variant thereof. In some instances, the ROCK inhibitor is at a concentration ranging from about 1 μM to about 10 μM. In some embodiments, the first culture medium and/or second culture medium are absent of animal serum.

Differentiation can be assayed as is known in the art, generally by evaluating the presence of RPE associated and/or specific markers or by measuring functionality. See, for example, Kamao et al., Stem Cell Reports, 2014, 2(2):205-18, the contents of which are herein incorporated by reference in its entirety.

Additional descriptions of RPE cells, including methods for their differentiation and for use in the present technology, are found in WO2020/018615, the disclosure of which is herein incorporated by reference in its entirety.

In some embodiments, the engineered RPE cells, such as primary RPE cells isolated from one or more individual donors (e.g., healthy donors) or RPE cells differentiated from iPSCs derived from one or more individual donors (e.g., healthy donors), are maintained in culture and in some cases expanded and/or cryopreserved. In certain embodiments, the population of RPE cells are cryopreserved.

Exemplary RPE cell types include, but are not limited to, retinal pigmented epithelium (RPE) cell, RPE progenitor cell, immature RPE cell, mature RPE cell, functional RPE cell, and the like.

In some embodiments, the RPE cells, such as primary RPE cells isolated from one or more individual donors (e.g., healthy donors) or RPE cells differentiated from iPSCs derived from one or more individual donors (e.g., healthy donors), have a genetic expression profile similar or substantially similar to that of native RPE cells. Such RPE cells may possess the polygonal, planar sheet morphology of native RPE cells when grown to confluence on a planar substrate.

In some embodiments, the present technology is directed to engineered RPE cells, such as primary RPE cells isolated from one or more individual donors (e.g., healthy donors) or RPE cells differentiated from iPSCs derived from one or more individual donors (e.g., healthy donors).

In some embodiments, the provided engineered RPE cells evade immune recognition. In some embodiments, the engineered RPE cells described herein, such as primary RPE cells isolated from one or more individual donors (e.g., healthy donors) or RPE cells differentiated from iPSCs derived from one or more individual donors (e.g., healthy donors), do not activate an immune response.

In some embodiments, the engineered cells or populations of cells provided herein are thyroid cells, such as primary thyroid cells. In some embodiments, the primary thyroid cells are isolated or obtained from one or more individual donor subjects, such as one or more individual healthy donor (e.g., a subject that is not known or suspected of, e.g., not exhibiting clinical signs of, a disease or infection). As will be appreciated by those skilled in the art, methods of isolating or obtaining thyroid cells from an individual can be achieved using known techniques.

In some embodiments, the engineered cells or populations of cells provided herein are thyroid cells, such as primary thyroid cells. In some embodiments, the primary thyroid cells are isolated or obtained from one or more individual donor subjects, such as one or more individual healthy donor (e.g., a subject that is not known or suspected of, e.g., not exhibiting clinical signs of, a disease or infection). As will be appreciated by those skilled in the art, methods of isolating or obtaining thyroid cells from an individual can be achieved using known techniques.

In some embodiments, primary thyroid cells are obtained (e.g., harvested, extracted, removed, or taken) from a subject or an individual. In some embodiments, primary thyroid cells are produced from a pool of thyroid cells such that the thyroid cells are from one or more subjects (e.g., one or more human including one or more healthy humans). In some embodiments, the pool of primary thyroid cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects.

In some embodiments, the cells as provided herein are thyroid cells differentiated from engineered iPSCs that contain modifications (e.g., genetic modifications) described herein and that are differentiated into a thyroid cell. As will be appreciated by those skilled in the art, the methods for differentiation depend on the desired cell type using known techniques.

In some embodiments, engineered pluripotent cells containing any of the vectors described herein are differentiated into thyroid progenitor cells and thyroid follicular organoids that can secrete thyroid hormones. Techniques to differentiate thyroid cells are known the art. See, e.g., Kurmann et al., Cell Stem Cell, 2015 Nov. 5; 17(5):527-42, incorporated herein by reference in its entirety and specifically for the methods and reagents for the generation of thyroid cells from human pluripotent stem cells. Differentiation can be assayed as is known in the art, generally by evaluating the presence of thyroid cell associated or specific markers or by measuring functionality.

In some embodiments, the population of engineered thyroid cells, such as primary thyroid cells isolated from one or more individual donors (e.g., healthy donors) or thyroid cells differentiated from iPSCs derived from one or more individual donors (e.g., healthy donors), are maintained in culture and in some cases expanded and/or cryopreserved. In certain embodiments, the thyroid cells are cryopreserved.

In some embodiments, the present technology is directed to engineered thyroid cells, such as primary thyroid cells isolated from one or more individual donors (e.g., healthy donors) or thyroid cells differentiated from iPSCs derived from one or more individual donors (e.g., healthy donors).

In some embodiments, the provided engineered thyroid cells evade immune recognition. In some embodiments, the engineered thyroid cells described herein, such as primary thyroid cells isolated from one or more individual donors (e.g., healthy donors) or thyroid cells differentiated from iPSCs derived from one or more individual donors (e.g., healthy donors), do not activate an immune response.

### Cardiac Cells

In some embodiments, the engineered cells or populations of cells provided herein are cardiac cells. In some embodiments, the engineered cardiac cells comprise cells differentiated from HIP cells comprising any of the vectors as described herein. As will be appreciated by those skilled in the art, the methods for differentiation depend on the desired cell type using known techniques. Exemplary cardiac cell types include, but are not limited to, a cardiomyocyte, nodal cardiomyocyte, conducting cardiomyocyte, working cardiomyocyte, cardiomyocyte precursor cell, cardiomyocyte progenitor cell, cardiac stem cell, cardiac muscle cell, atrial cardiac stem cell, ventricular cardiac stem cell, epicardial cell, hematopoietic cell, vascular endothelial cell, endocardial endothelial cell, cardiac valve interstitial cell, cardiac pacemaker cell, and the like.

In some embodiments, cardiomyocyte precursor cells include a cell that is capable of giving rise to progeny that include mature (end-stage) cardiomyocytes. Cardiomyocyte precursor cells can often be identified using one or more markers selected from GATA-4, Nkx2.5, and the MEF-2 family of transcription factors. In some instances, cardiomyocytes refer to immature cardiomyocytes or mature cardiomyocytes that express one or more markers (sometimes at least 2, 3, 4, or 5 markers) from the following list: cardiac troponin I (cTn1), cardiac troponin T (cTnT), sarcomeric myosin heavy chain (MHC), GATA-4, Nkx2.5, N-cadherin, β2-adrenoceptor, ANF, the MEF-2 family of transcription factors, creatine kinase MB (CK-MB), myoglobin, and atrial natriuretic factor (ANF). In some embodiments, the cardiac cells of the disclosure demonstrate spontaneous periodic contractile activity. In some cases, when those cardiac cells are cultured in a suitable tissue culture environment with an appropriate Ca²⁺ concentration and electrolyte balance, the cells can be observed to contract in a periodic fashion across one axis of the cell, and then release from contraction, without having to add any additional components to the culture medium. In some embodiments, the cardiac cells of the disclosure are hypoimmunogenic cardiac cells.

In some embodiments, the method of producing hypoimmunogenic cardiac cells from hypoimmunogenic pluripotent (HIP) cells by in vitro differentiation comprises: (a) culturing HIP cells in a culture medium comprising a GSK inhibitor; (b) culturing the HIP cells in a culture medium comprising a WNT antagonist to produce a population of pre-cardiac cells; and (c) culturing the pre-cardiac cells in a culture medium comprising insulin to produce hypoimmune cardiac cells. In some embodiments, the GSK inhibitor is CHIR-99021, a derivative thereof, or a variant thereof. In some instances, the GSK inhibitor is at a concentration ranging from about 2 mM to about 10 mM. In some embodiments, the WNT antagonist is IWR1, a derivative thereof, or a variant thereof. In some instances, the WNT antagonist is at a concentration ranging from about 2 mM to about 10 mM.

In some embodiments, the hypoimmunogenic cardiac cells is isolated from non-cardiac cells. In some embodiments, the isolated hypoimmunogenic cardiac cells are expanded. In certain embodiments, the isolated hypoimmunogenic cardiac cells are expanded and cryopreserved.

Other useful methods for differentiating induced pluripotent stem cells or pluripotent stem cells into cardiac cells are described, for example, in US2017/0152485; US2017/0058263; US2017/0002325; US2016/0362661; US2016/0068814; U.S. Pat. Nos. 9,062,289; 7,897,389; and 7,452,718. Additional methods for producing cardiac cells from induced pluripotent stem cells or pluripotent stem cells are described in, for example, Xu et al., Stem Cells and Development, 2006, 15(5): 631-9, Burridge et al., Cell Stem Cell, 2012, 10: 16-28, and Chen et al., Stem Cell Res, 2015, 15(2):365-375.

In various embodiments, hypoimmunogenic cardiac cells can be cultured in medium comprising a BMP pathway inhibitor, a WNT signaling activator, a WNT signaling inhibitor, a WNT agonist, a WNT antagonist, a Src inhibitor, a EGFR inhibitor, a PCK activator, a cytokine, a growth factor, a cardiotropic agent, a compound, and/or the like.

The WNT signaling activator includes, but is not limited to, CHIR99021. The PCK activator includes, but is not limited to, PMA. The WNT signaling inhibitor includes, but is not limited to, a compound selected from KY02111, S03031 (KY01-I), S02031 (KY02-I), and S03042 (KY03-I), and XAV939. The Src inhibitor includes, but is not limited to, A419259. The EGFR inhibitor includes, but is not limited to, AG1478.

Non-limiting examples of an agent for generating a cardiac cell from an iPSC include activin A, BMP4, Wnt3a, VEGF, soluble frizzled protein, cyclosporin A, angiotensin II, phenylephrine, ascorbic acid, dimethylsulfoxide, 5-aza-2′-deoxycytidine, and the like.

The cells provided herein can be cultured on a surface, such as a synthetic surface to support and/or promote differentiation of hypoimmunogenic pluripotent cells into cardiac cells. In some embodiments, the surface comprises a polymer material including, but not limited to, a homopolymer or copolymer of selected one or more acrylate monomers. Non-limiting examples of acrylate monomers and methacrylate monomers include tetra(ethylene glycol) diacrylate, glycerol dimethacrylate, 1,4-butanediol dimethacrylate, poly(ethylene glycol) diacrylate, di(ethylene glycol) dimethacrylate, tetra(ethyiene glycol) dimethacrylate, 1,6-hexanediol propoxylate diacrylate, neopentyl glycol diacrylate, trimethylolpropane benzoate diacrylate, trimethylolpropane eihoxylate (1 EO/QH) methyl, tricyclo[5.2.1.02,6]decane dimethanol diacrylate, neopentyl glycol exhoxylate diacrylate, and trimethylolpropane triacrylate. Acrylate synthesized is known in the art or obtained from a commercial vendor, such as Polysciences, Inc., Sigma Aldrich, Inc. and Sartomer, Inc.

The polymeric material can be dispersed on the surface of a support material. Useful support materials suitable for culturing cells include a ceramic substance, a glass, a plastic, a polymer or co-polymer, any combinations thereof, or a coating of one material on another. In some instances, a glass includes soda-lime glass, pyrex glass, vycor glass, quartz glass, silicon, derivatives of these, or the like.

In some instances, plastics or polymers including dendritic polymers include poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine, derivatives of these, or the like. In some instances, copolymers include poly(vinyl acetate-co-maleic anhydride), poly(styrene-co-maleic anhydride), poly(ethylene-co-acrylic acid), derivatives of these, or the like.

The efficacy of cardiac cells prepared as described herein can be assessed in animal models for cardiac cryoinjury, which causes 55% of the left ventricular wall tissue to become scar tissue without treatment (Li et al., Ann. Thorac. Surg. 62:654, 1996; Sakai et al., Ann. Thorac. Surg. 8:2074, 1999, Sakai et al., Thorac. Cardiovasc. Surg. 118:715, 1999).

In some embodiments, the engineered cardiac cells, such cardiac cells differentiated from iPSCs derived from one or more individual donors (e.g., healthy donors), are maintained in culture and in some cases expanded and/or cryopreserved. In certain embodiments, the cardiac cells are cryopreserved.

In some embodiments, the present technology is directed to engineered cardiac cells, such as cardiac cells differentiated from iPSCs derived from one or more individual donors (e.g., healthy donors).

In some embodiments, the provided engineered cardiac cells evade immune recognition. In some embodiments, the engineered cardiac cells described herein, such as cardiac cells differentiated from iPSCs derived from one or more individual donors (e.g., healthy donors), do not activate an immune response.

### Neural Cells

In some embodiments, the engineered cells or populations of cells provided herein are neural cells, including neural cell types differentiated from engineered pluripotent cells (e.g., iPSCs) as described herein. As will be appreciated by those skilled in the art, the methods for differentiation depend on the desired cell type using known techniques. Exemplary neural cell types include, but are not limited to, cerebral endothelial cells, neurons (e.g., dopaminergic neurons), glial cells, and the like.

In some embodiments, differentiation of induced pluripotent stem cells is performed by exposing or contacting cells to specific factors which are known to produce a specific cell lineage(s), so as to target their differentiation to a specific, desired lineage and/or cell type of interest. In some embodiments, terminally differentiated cells display specialized phenotypic characteristics or features. In certain embodiments, the stem cells described herein are differentiated into a neuroectodermal, neuronal, neuroendocrine, dopaminergic, cholinergic, serotonergic (5-HT), glutamatergic, GABAergic, adrenergic, noradrenergic, sympathetic neuronal, parasympathetic neuronal, sympathetic peripheral neuronal, or glial cell population. In some instances, the glial cell population includes a microglial (e.g., amoeboid, ramified, activated phagocytic, and activated non-phagocytic) cell population or a macroglial (e.g., central nervous system cell: astrocyte, oligodendrocyte, ependymal cell, and radial glia; and peripheral nervous system cell: Schwann cell and satellite cell) cell population, or the precursors and progenitors of any of the preceding cells.

Protocols for generating different types of neural cells are described in PCT Application No. WO2010144696, U.S. Pat. Nos. 9,057,053; 9,376,664; and 10,233,422. Additional descriptions of methods for differentiating hypoimmunogenic pluripotent cells can be found, for example, in Deuse et al., Nature Biotechnology, 2019, 37, 252-258 and Han et al., Proc Natl Acad Sci USA, 2019, 116(21), 10441-10446.

In some embodiments, the engineered neural cells, such as neural cells differentiated from iPSCs derived from one or more individual donors (e.g., healthy donors), are maintained in culture and in some cases expanded and/or cryopreserved. In certain embodiments, the neural cells are cryopreserved.

In some embodiments, the present disclosure is directed to engineered neural cells, such as neural cells differentiated from iPSCs derived from one or more individual donors (e.g., healthy donors), that is engineered to express any of the vectors described herein.

In some embodiments, the provided engineered neural cells evade immune recognition. In some embodiments, the engineered neural cells described herein, such as neural cells differentiated from iPSCs derived from one or more individual donors (e.g., healthy donors), do not activate an immune response.

In some embodiments, the engineered cells or populations of cells provided herein are cerebral endothelial cells (ECs), precursors, and/or progenitors thereof, e.g., differentiated from pluripotent stem cells (e.g., induced pluripotent stem cells). In some embodiments, the cerebral endothelial cells (ECs), precursors, and/or progenitors thereof are differentiated from pluripotent stem cells on a surface by culturing the cells in a medium comprising one or more factors that promote the generation of cerebral ECs or neural cell. In some instances, the medium includes one or more of the following: CHIR-99021, VEGF, basic FGF (bFGF), or Y-27632. In some embodiments, the medium includes a supplement designed to promote survival and functionality for neural cells. In some embodiments, cerebral endothelial cells (ECs), precursors, and progenitors thereof are differentiated from pluripotent stem cells on a surface by culturing the cells in an unconditioned or conditioned medium. In some instances, the medium comprises factors or small molecules that promote or facilitate differentiation. In some embodiments, the medium comprises one or more factors or small molecules selected from the group consisting of VEGR, FGF, SDF-1, CHIR-99021, Y-27632, SB 431542, and any combination thereof. In some embodiments, the surface for differentiation comprises one or more extracellular matrix proteins. The surface can be coated with the one or more extracellular matrix proteins. The cells can be differentiated in suspension and then put into a gel matrix form, such as matrigel, gelatin, or fibrin/thrombin forms to facilitate cell survival. In some cases, differentiation is assayed as is known in the art, generally by evaluating the presence of cell-specific markers. In some embodiments, the cerebral endothelial cells express or secrete a factor selected from CD31, VE cadherin, and any combinations thereof. In certain embodiments, the cerebral endothelial cells express or secrete one or more of the factors selected from CD31, CD34, CD45, CD117 (c-kit), CD146, CXCR4, VEGF, SDF-1, PDGF, GLUT-1, PECAM-1, eNOS, claudin-5, occludin, ZO-1, p-glycoprotein, von Willebrand factor, VE-cadherin, low density lipoprotein receptor LDLR, low density lipoprotein receptor-related protein 1 LRP1, insulin receptor INSR, leptin receptor LEPR, basal cell adhesion molecule BCAM, transferrin receptor TFRC, advanced glycation end product-specific receptor AGER, receptor for retinol uptake STRA6, large neutral amino acids transporter small subunit 1 SLC7A5, excitatory amino acid transporter 3 SLC1A1, sodium-coupled neutral amino acid transporter 5 SLC38A5, solute carrier family 16 member 1 SLC16A1, ATP-dependent translocase ABCB1, ATP-ABCC2-binding cassette transporter ABCG2, multidrug resistance-associated protein 1 ABCC1, canalicular multispecific organic anion transporter 1 ABCC2, multidrug resistance-associated protein 4 ABCC4, multidrug resistance-associated protein 5 ABCC5, or any combination thereof. In some embodiments, the cerebral ECs are characterized with one or more of the features selected from high expression of tight junctions, high electrical resistance, low fenestration, small perivascular space, high prevalence of insulin and transferrin receptors, high number of mitochondria, or any combination thereof. In some embodiments, cerebral ECs are selected or purified using a positive selection strategy. In some instances, the cerebral ECs are sorted against an endothelial cell marker such as, but not limited to, CD31. In other words, CD31 positive cerebral ECs are isolated. In some embodiments, cerebral ECs are selected or purified using a negative selection strategy. In some embodiments, undifferentiated or pluripotent stem cells are removed by selecting for cells that express a pluripotency marker including, but not limited to, TRA-1-60 and SSEA-1.

In some embodiments, the engineered cells or population of cells provided herein are dopaminergic neurons, including neuronal stem cells, neuronal progenitor cells, immature dopaminergic neurons, and mature dopaminergic neurons. In some embodiments, the dopaminergic neurons are differentiated from HIP cells. In some cases, dopaminergic neurons include neuronal cells which express tyrosine hydroxylase (TH), the rate-limiting enzyme for dopamine synthesis. In some embodiments, dopaminergic neurons secrete the neurotransmitter dopamine, and have little or no expression of dopamine hydroxylase. A dopaminergic (DA) neuron can express one or more of the following markers: neuron-specific enolase (NSE), 1-aromatic amino acid decarboxylase, vesicular monoamine transporter 2, dopamine transporter, Nurr-1, or dopamine-2 receptor (D2 receptor). In certain cases, neural stem cells include a population of pluripotent cells that have partially differentiated along a neural cell pathway and express one or more neural markers including, for example, nestin. Neural stem cells may differentiate into neurons or glial cells (e.g., astrocytes and oligodendrocytes). Neural progenitor cells can include cultured cells which express FOXA2 and low levels of 0-tubulin, but not tyrosine hydroxylase. Such neural progenitor cells have the capacity to differentiate into a variety of neuronal subtypes; particularly a variety of dopaminergic neuronal subtypes, upon culturing the appropriate factors, such as those described herein. In some embodiments, DA neurons, precursors, and progenitors thereof are differentiated from pluripotent stem cells by culturing the stem cells in medium comprising one or more factors or additives. Useful factors and additives that promote differentiation, growth, expansion, maintenance, and/or maturation of DA neurons include, but are not limited to, Wnt1, FGF2, FGF8, FGF8a, sonic hedgehog (SHH), brain derived neurotrophic factor (BDNF), transforming growth factor α (TGF-α), TGF-β, interleukin 1 beta, glial cell line-derived neurotrophic factor (GDNF), a GSK-3 inhibitor (e.g., CHIR-99021), a TGF-β inhibitor (e.g., SB-431542), B-27 supplement, dorsomorphin, purmorphamine, noggin, retinoic acid, cAMP, ascorbic acid, neurturin, knockout serum replacement, N-acetyl cysteine, c-kit ligand, modified forms thereof, mimics thereof, analogs thereof, or variants thereof. In some embodiments, the DA neurons are differentiated in the presence of one or more factors that activate or inhibit the WNT pathway, NOTCH pathway, SHH pathway, BMP pathway, FGF pathway, and the like. Differentiation protocols and detailed descriptions thereof are provided in, e.g., U.S. Pat. Nos. 9,968,637, 7,674,620, Kim et al., Nature, 2002, 418,50-56; Bjorklund et al., PNAS, 2002, 99(4), 2344-2349; Grow et al., Stem Cells Transl Med. 2016, 5(9): 1133-44, and Cho et al., PNAS, 2008, 105:3392-3397, the disclosures in their entirety including the detailed description of the examples, methods, figures, and results are herein incorporated by reference. In some embodiments, hypoimmunogenic dopaminergic neurons can be isolated from non-neuronal cells. In some embodiments, isolated hypoimmunogenic dopaminergic neurons can be expanded. In certain embodiments, the isolated hypoimmunogenic dopaminergic neurons are expanded and cryopreserved. To characterize and monitor DA differentiation and assess the DA phenotype, expression of any number of molecular and genetic markers can be evaluated. For example, the presence of genetic markers can be determined by various methods known to those skilled in the art. Expression of molecular markers can be determined by quantifying methods such as, but not limited to, qPCR-based assays, immunoassays, immunocytochemistry assays, immunoblotting assays, or the like. Exemplary markers for DA neurons include, but are not limited to, TH, b-tubulin, paired box protein (Pax6), insulin gene enhancer protein (Isl1), nestin, diaminobenzidine (DAB), G protein-activated inward rectifier potassium channel 2 (GIRK2), microtubule-associated protein 2 (MAP-2), NURR1, dopamine transporter (DAT), forkhead box protein A2 (FOXA2), FOX3, doublecortin, LIM homeobox transcription factor 1-beta (LMX1B), or the like. In some embodiments, the DA neurons express one or more of the markers selected from corin, FOXA2, TuJI, NURR1, or any combination thereof. In some embodiments, DA neurons are assessed according to cell electrophysiological activity. The electrophysiology of the cells can be evaluated by using assays known to those skilled in the art. For instance, whole-cell and perforated patch clamp, assays for detecting electrophysiological activity of cells, assays for measuring the magnitude and duration of action potential of cells, and functional assays for detecting dopamine production of DA cells. In some embodiments, DA neuron differentiation is characterized by spontaneous rhythmic action potentials, and high-frequency action potentials with spike frequency adaption upon injection of depolarizing current. In other embodiments, DA differentiation is characterized by the production of dopamine. The level of dopamine produced is calculated by measuring the width of an action potential at the point at which it has reached half of its maximum amplitude (spike half-maximal width). Useful descriptions of neurons derived from stem cells and methods of making thereof can be found, for example, in Kirkeby et al., Cell Rep, 2012, 1:703-714; Kriks et al., Nature, 2011, 480:547-551; Wang et al., Stem Cell Reports, 2018, 11(1):171-182; Lorenz Studer, “Chapter 8—Strategies for Bringing Stem Cell-Derived Dopamine Neurons to the clinic—The NYSTEM Trial” in Progress in Brain Research, 2017, volume 230, pg. 191-212; Liu et al., Nat Protoc, 2013, 8:1670-1679; Upadhya et al., Curr Protoc Stem Cell Biol, 38, 2D.7.1-2D.7.47; US Publication Appl. No. 20160115448, and U.S. Pat. Nos. 8,252,586; 8,273,570; 9,487,752 and 10,093,897, the contents are incorporated herein by reference in their entirety. In addition to DA neurons, other neuronal cells, precursors, and progenitors thereof can be differentiated from the HIP cells outlined herein by culturing the cells in medium comprising one or more factors or additives. Non-limiting examples of factors and additives include GDNF, BDNF, GM-CSF, B27, basic FGF, basic EGF, NGF, CNTF, SMAD inhibitor, Wnt antagonist, SHH signaling activator, or any combination thereof. In some embodiments, the SMAD inhibitor is selected from the group consisting of SB431542, LDN-193189, Noggin PD169316, SB203580, LY364947, A77-01, A-83-01, BMP4, GW788388, GW6604, SB-505124, lerdelimumab, metelimumab, GC-I008, AP-12009, AP-110I4, LY550410, LY580276, LY364947, LY2109761, SB-505124, E-616452 (RepSox ALK inhibitor), SD-208, SMI6, NPC-30345, K 26894, SB-203580, SD-093, activin-M108A, P144, soluble TBR2-Fc, DMH-1, dorsomorphin dihydrochloride, or derivatives thereof. In some embodiments, the Wnt antagonist is selected from the group consisting of XAV939, DKK1, DKK-2, DKK-3, DKK-4, SFRP-1, SFRP-2, SFRP-3, SFRP-4, SFRP-5, WIF-1, Soggy, IWP-2, IWR1, ICG-001, KY0211, Wnt-059, LGK974, IWP-L6, and derivatives thereof. In some embodiments, the SHH signaling activator is selected from the group consisting of Smoothened agonist (SAG), SAG analog, SHH, C25-SHH, C24-SHH, purmorphamine, Hg-Ag, and derivatives thereof. In some embodiments, the neurons express one or more of the markers selected from the group consisting of glutamate ionotropic receptor NMDA type subunit 1 GRIN1, glutamate decarboxylase 1 GAD1, gamma-aminobutyric acid GABA, tyrosine hydroxylase TH, LIM homeobox transcription factor 1-alpha LMX1A, Forkhead box protein 01 FOXO1, Forkhead box protein A2 FOXA2, Forkhead box protein 04 FOXO4, FOXG1, 2′,3′-cyclic-nucleotide 3′-phosphodiesterase CNP, myelin basic protein MBP, tubulin beta chain 3 TUB3, tubulin beta chain 3 NEUN, solute carrier family 1 member 6 SLC1A6, SST, PV, calbindin, RAX, LHX6, LHX8, DLX1, DLX2, DLX5, DLX6, SOX6, MAFB, NPAS1, ASCL1, SIX6, OLIG2, NKX2.1, NKX2.2, NKX6.2, VGLUT1, MAP2, CTIP2, SATB2, TBR1, DLX2, ASCL1, ChAT, NGFI-B, c-fos, CRF, RAX, POMC, hypocretin, NADPH, NGF, Ach, VAChT, PAX6, EMX2p75, CORIN, TUJ1, NURR1, and any combination thereof.

In some embodiments, the engineered cells or population of cells provided herein are glial cells such as, but not limited to, microglia, astrocytes, oligodendrocytes, ependymal cells and Schwann cells, glial precursors, and glial progenitors thereof that are produced by differentiating pluripotent stem cells into therapeutically effective glial cells, and the like. Differentiation of hypoimmunogenic pluripotent stem cells produces hypoimmunogenic neural cells, such as hypoimmunogenic glial cells. In some embodiments, glial cells, precursors, and progenitors thereof can be generated by culturing pluripotent stem cells in medium comprising one or more agents selected from the group consisting of retinoic acid, IL-34, M-CSF, FLT3 ligand, GM-CSF, CCL2, a TGFbeta inhibitor, a BMP signaling inhibitor, a SHH signaling activator, FGF, platelet derived growth factor PDGF, PDGFR-alpha, HGF, IGF1, noggin, SHH, dorsomorphin, noggin, and any combination thereof. In certain instances, the BMP signaling inhibitor is LDN193189, SB431542, or a combination thereof. In some embodiments, the glial cells express NKX2.2, PAX6, SOX10, brain derived neurotrophic factor BDNF, neutrotrophin-3 NT-3, NT-4, EGF, ciliary neurotrophic factor CNTF, nerve growth factor NGF, FGF8, EGFR, OLIG1, OLIG2, myelin basic protein MBP, GAP-43, LNGFR, nestin, GFAP, CD11b, CD11c, CX3CR1, P2RY12, IBA-1, TMEM119, CD45, or any combination thereof. Exemplary differentiation medium can include any specific factors and/or small molecules that may facilitate or enable the generation of a glial cell type as recognized by those skilled in the art. To determine if the cells generated according to the in vitro differentiation protocol display glial cell characteristics and features, the cells can be transplanted into an animal model. In some embodiments, the glial cells are injected into an immunocompromised mouse, e.g., an immunocompromised shiverer mouse. The glial cells are administered to the brain of the mouse and after a pre-selected amount of time, the engrafted cells are evaluated. In some instances, the engrafted cells in the brain are visualized by using immunostaining and imaging methods. In some embodiments, it is determined that the glial cells express known glial cell biomarkers. Useful methods for generating glial cells, precursors, and progenitors thereof from stem cells are found, for example, in U.S. Pat. Nos. 7,579,188; 7,595,194; 8,263,402; 8,206,699; 8,252,586; 9,193,951; 9,862,925; 8,227,247; 9,709,553; US2018/0187148; US2017/0198255; US2017/0183627; US2017/0182097; US2017/253856; US2018/0236004; WO2017/172976; and WO2018/093681. Methods for differentiating pluripotent stem cells are described in, e.g., Kikuchi et al., Nature, 2017, 548, 592-596; Kriks et al., Nature, 2011, 547-551; Doi et al., Stem Cell Reports, 2014, 2, 337-50; Perrier et al., Proc Natl Acad Sci USA, 2004, 101, 12543-12548; Chambers et al., Nat Biotechnol, 2009, 27, 275-280; and Kirkeby et al., Cell Reports, 2012, 1, 703-714. Additional descriptions of neural cells including dopaminergic neurons for use in the present technology are found in WO2020/018615, the disclosure of which is herein incorporated by reference in its entirety.

### Hematopoietic Stem Cells

In some embodiments, the engineered cells or populations of cells provided herein are hematopoietic stem cells. In some cases, the hematopoietic stem cell is an immature cell that can develop into all types of blood cells, including white blood cells, red blood cells, and platelets. Hematopoietic stem cells (HSC) are found in the peripheral blood and the bone marrow. In some cases, the hematopoietic stem cell is isolated from the peripheral blood or bone marrow.

ii. Producer Cells

An engineered cell or population of cells of the present disclosure may find use in the production of molecules of interest, such as antibodies, fusogens, and viral or virus-like particles. In some cases, the engineered cells or populations of cells may be used for the production of one or more proteins encoded by a transgene described herein, e.g., kill switch and/or genome editing complex. Non-limiting examples of cells that may be used include CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, NS0, PerC6, Sp2/0, BHK, C127, and 211 A cells. In some embodiments, the engineered cell or population of cells of the disclosure is a HEK293 cell, e.g., a HEK293T cell. In some embodiments, the engineered cell or population of cells of the disclosure is a HeLa cell. In some embodiments, the engineered cell or population of cells of the disclosure is a NS0 cell. In some embodiments, the engineered cell or population of cells of the disclosure is a PerC6 cell.

In some embodiments, engineered cells or population of cells of the present disclosure may be used for the production of virus or virus-like particles. In such cases, multiple cell culture systems including bacteria, mammalian cell lines, insect cell lines, yeast, and plant cells can be used. In some embodiments, elements for the production of a viral or virus-like particle, e.g., a recombinant viral particle such as a replication incompetent lentiviral vector, are included in a packaging cell line or are present on a packaging vector. In some embodiments, viral particles can include packaging elements, rev, gag, and pol, delivered to the packaging cell line via one or more packaging vectors. In some embodiments, the packaging vector is an expression vector or viral vector that lacks a packaging signal and comprises a polynucleotide encoding one, two, three, four, or more viral structural and/or accessory genes. Typically, the packaging vectors are included in a packaging cell, and are introduced into the cell via transfection, transduction, or infection. A retroviral, e.g., lentiviral, transfer vector can be introduced into a packaging cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase, or ADA, followed by selection in the presence of the appropriate drug and isolation of clones. A selectable marker gene can be linked physically to genes encoded by the packaging vector, e.g., by IRES or self-cleaving viral peptides. In some embodiments, the packaging vector is a packaging plasmid.

In some embodiments, engineered cells or population of cells of the disclosure comprise cells from producer (or packaging) cell lines. In some embodiments, the producer cell line further stably expresses one or more transgene sequences encoded by any of the vectors described herein. Suitable cell lines that can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, HEK293 cells, HEK293T cells, B-50 cells, 3T3 cells, NIH3T3 cells, HepG2 cells, Saos-2 cells, Huh7 cells, HeLa cells, W163 cells, PerC6 cells, NS0 cells, 211 cells, and 211 A cells. In embodiments, the packaging cells are HEK293 cells, HEK293T cells, HeLa cells, PerC6 cells, or NS0 cells. In some embodiments, producer cells (e.g., a source cell line) include a cell line that is capable of producing recombinant retroviral particles, comprising a packaging cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al., (1992) J. Virol. 66:5110-5113, which are incorporated herein by reference. Infectious virus particles may be collected from the packaging cells, e.g., by cell lysis, or collection of the supernatant of the cell culture. Optionally, the collected virus particles may be enriched or purified. In some embodiments, the source cell comprises one or more plasmids coding for viral structural proteins and replication enzymes (e.g., gag, pol, and env) that can package viral particles (i.e., a packaging plasmid). In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector. In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level. In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al., 1998; Jones et al., 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, engineered cells or populations of cells (e.g., T cells, B cells, NK cells, islet cells such as beta cells, or hypoimmunogenic cells thereof) of the present disclosure may be used for the production of retroviruses such as lentiviruses.

In some embodiments, third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments, a nucleic acid encoding the exogenous agent (e.g., a retroviral nucleic acid encoding the vector comprising one or more transgene) is also integrated into the source cell genome. In some embodiments a nucleic acid encoding the vector comprising one or more transgene is maintained episomally. In some embodiments a nucleic acid encoding the vector comprising one or more transgene is transfected into the source cell that has stably integrated Rev, Gag/Pol, and an envelope protein in the genome. See, e.g., Milani et al., EMBO Molecular Medicine, 2017, which is herein incorporated by reference in its entirety.

In some embodiments, a retroviral nucleic acid described herein is unable to undergo reverse transcription. Such a nucleic acid, in some embodiments, is able to transiently express a vector comprising one or more transgene. The retrovirus may comprise a disabled reverse transcriptase protein, or may not comprise a reverse transcriptase protein. In some embodiments, the retroviral nucleic acid comprises a disabled primer binding site (PBS) and/or att site. In some embodiments, one or more viral accessory genes, including rev, tat, vif, nef, vpr, vpu, vpx, S2, or functional equivalents thereof, are disabled or absent from the retroviral nucleic acid. In embodiments, one or more accessory genes selected from S2, rev, and tat are disabled or absent from the retroviral nucleic acid.

Typically, modern retroviral vector systems include viral genomes bearing cis-acting vector sequences for transcription, reverse-transcription, integration, translation, and packaging of viral RNA into the viral particles, and producer cells lines which express the trans-acting retroviral gene sequences (e.g., gag, pol, and env) needed for production of virus particles. By separating the cis- and trans-acting vector sequences completely, the virus is unable to maintain replication for more than one cycle of infection. Generation of live virus can be avoided by a number of strategies, e.g., by minimizing the overlap between the cis- and trans-acting sequences to avoid recombination.

In some embodiments, engineered cells or populations of cells (e.g., T cells, B cells, NK cells, islet cells such as beta cells, or hypoimmunogenic cells thereof) of the present disclosure may be used for the production of virus or virus-like particles (VLPs). A virus-like particle (VLP) which comprises a sequence that is devoid of or lacking viral genetic material may be the result of removing or eliminating the viral RNA from the sequence. Similar to viral particles, VLPs contain a viral outer envelope made from the host cell (i.e., producer cell or source cell) lipid-bilayer as well as at least one viral structural protein. In some embodiments, a viral structural protein refers to any viral protein or fragment thereof which contributes to the structure of the viral core or capsid. Generally, for viral particles, expression of the gag precursor protein alone mediates vector assembly and release. In some aspects, gag proteins or fragments thereof have been demonstrated to assemble into structures analogous to viral cores. In one embodiment this may be achieved by using an endogenous packaging signal binding site on gag. Alternatively, the endogenous packaging signal binding site is on pol. In this embodiment, the RNA which is to be delivered will contain a cognate packaging signal. In another embodiment, a heterologous binding domain (which is heterologous to gag) located on the RNA to be delivered, and a cognate binding site located on gag or pol, can be used to ensure packaging of the RNA to be delivered. The heterologous sequence could be non-viral or it could be viral, in which case it may be derived from a different virus. The VLP could be used to deliver therapeutic RNA, in which case functional integrase and/or reverse transcriptase is not required. These VLPs could also be used to deliver a therapeutic gene of interest, in which case pol is typically included.

In an embodiment, gag-pol are altered, and the packaging signal is replaced with a corresponding packaging signal. In this embodiment, the particle can package the RNA with the new packaging signal. The advantage of this approach is that it is possible to package an RNA sequence that is devoid of viral sequence for example, RNAi.

An alternative approach is to rely on over-expression of the RNA to be packaged. In one embodiment the RNA to be packaged is over-expressed in the absence of any RNA containing a packaging signal. This may result in a significant level of therapeutic RNA being packaged, and that this amount is sufficient to transduce a cell and have a biological effect.

In some embodiments, a polynucleotide, such as a transgene, comprises a nucleotide sequence encoding a viral gag protein or retroviral gag and pol proteins, wherein the gag protein or pol protein comprises a heterologous RNA binding domain capable of recognizing a corresponding sequence in an RNA sequence to facilitate packaging of the RNA sequence into a viral vector particle. In some embodiments, the heterologous RNA binding domain comprises an RNA binding domain derived from a bacteriophage coat protein, a Rev protein, a protein of the U1 small nuclear ribonucleoprotein particle, a Nova protein, a TF111 A protein, a TIS 11 protein, a trp RNA-binding attenuation protein (TRAP), or a pseudouridine synthase.

In some embodiments, the assembly of a viral based vector vehicle particle (i.e., a VLP) is initiated by binding of the core protein to a unique encapsidation sequence within the viral genome (e.g., UTR with stem-loop structure). In some embodiments, the interaction of the core with the encapsidation sequence facilitates oligomerization.

In some embodiments, the source cell for VLP production comprises one or more plasmids coding for viral structural proteins (e.g., gag, pol) which can package viral particles (i.e., a packaging plasmid). In some embodiments, the sequences coding for at least two of the gag and pol precursors are on the same plasmid. In some embodiments, the sequences coding for the gag and pol precursors are on different plasmids. In some embodiments, the sequences coding for the gag and pol precursors have the same expression signal, e.g., promoter. In some embodiments, the sequences coding for the gag and pol precursors have a different expression signal, e.g., different promoters. In some embodiments, expression of the gag and pol precursors is inducible.

In some embodiments, formation of VLPs or any viral-based particle can be detected by any suitable technique known in the art. Examples of such techniques include, e.g., electron microscopy, dynamic light scattering, selective chromatographic separation, and/or density gradient centrifugation.

In some embodiments, the cell is a cell line, such as CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, NS0, PerC6, Sp2/0, BHK, C127 and 211 A cells.

iii. Samples

In some embodiments, the sample is a biological sample. In some embodiments, the sample is from a human. In some embodiments, the sample comprises an engineered cell or population of cells (e.g., T cells, B cells, NK cells, islet cells such as beta cells, or hypoimmunogenic cells thereof). In some embodiments, the comprises a population of cells. In some embodiments, the sample comprises any cell described herein. In some embodiments, the sample comprises any population of cells described herein. In some embodiments, the sample is a tissue sample, and may include, for example, tissue sample, biopsy, resection, smear, aspirate, PBMC, cell lines, hair follicles, or any combination thereof.

The disclosed method may be used with any of a variety of samples comprising cells that are collected from a subject (e.g., a patient). Examples of a sample include, but are not limited to, a liquid biopsy sample, a blood sample (e.g., a peripheral whole blood sample), a blood plasma sample, a blood serum sample, a lymph sample, a circulating tumor cell (CTC) sample, a cerebrospinal fluid (CSF) sample, a pericardial fluid sample, a pleural fluid sample, an ascites (peritoneal fluid) sample, an interstitial fluid sample, a feces (or stool) sample, or other body fluid, secretion, and/or excretion sample that can comprise a cell of interest (or sample derived therefrom), as well as, e.g., a tissue sample, biopsy, resection, smear, or aspirate. A tissue sample may be harvested from any tissue in the body, e.g., lung, liver, pancreas, bone, gynecological, cardiac, muscle, adipose, brain, gastrointestinal, breast, eye, cartilage, tumor, etc.

In some embodiments, the sample may be collected by surgical resection, needle biopsy, fine needle aspiration, collection cup or tube, oral swab, nasal swab, vaginal swab or a cytology smear, etc. In some embodiments, the sample comprises a mixture of single cells. In some embodiments, the sample is further processed, e.g., digested, dissected, or otherwise broken down into a single cell mixture. In some embodiments, the sample is further processed to select for cells of a specific lineage or phenotype, for example a T cell, B cell, NK cell, macrophage, monocyte, dendritic cell, pancreatic islet cell (e.g., beta cell), keratinocyte, hepatocyte, stellate cell, cardiac cell, etc. wherein the cell presents specific lineage markers and can be selected or sorted, for example by magnetic-activated cell selection (MACS) or fluorescent-activated cell sorting (FACS).

In some embodiments, the sample cell population is at least about 60% pure, e.g., at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% pure cell population, wherein a “pure” cell population comprises a population of cells of one cell type or one cell lineage, for example a population of: CD8+ cytotoxic T cells; regulatory T cells (Tregs); CD4+TH1 cells; CD4+TH2 cells; hepatocytes; stellate cells; keratinocytes; natural killer (NK) cells; beta cells; etc.

### F. Assays for Hypoimmunogenic Phenotypes

In some embodiments, the engineered primary cells provided herein can be assessed or assessed for hypoimmunogenecity. In some embodiments, hypoimmunogenecity of a cell can be determined by evaluating the immunogenicity of the cell such as the cell's ability to elicit adaptive and innate immune responses. Such immune response can be measured using assays recognized by those skilled in the art. In some embodiments, an immune response assay measures the effect of a hypoimmunogenic cell on T cell proliferation, T cell activation, T cell killing, NK cell proliferation, NK cell activation, and macrophage activity. In some cases, hypoimmunogenic cells and derivatives thereof undergo decreased killing by T cells and/or NK cells upon administration to a subject. In some cases, hypoimmunogenic cells and derivatives thereof undergo decreased killing by macrophages upon administration to a subject. In some cases, hypoimmunogenic cells and derivatives thereof undergo decreased killing by peripheral blood mononuclear cells (PBMCs) upon administration to a subject. In some instances, the cells and derivatives thereof show decreased macrophage engulfment compared to an unmodified or wildtype cell. In some embodiments, a hypoimmunogenic cell elicits a reduced or diminished immune response in a recipient subject compared to a corresponding unmodified wild-type cell. In some embodiments, a hypoimmunogenic cell is nonimmunogenic or fails to elicit an immune response in a recipient subject.

Once the hypoimmunogenic cells have been generated, they may be assayed for their hypoimmunogenicity, engraftment, and function, as is described in WO2016183041 and WO2018132783.

The hypoimmunogenic cells are administered in a manner that permits them to engraft to the intended tissue site and reconstitute or regenerate the functionally deficient area. In some embodiments, the hypoimmunogenic cells are assayed for engraftment (e.g., successful engraftment). In some embodiments, the engraftment of the hypoimmunogenic cells is evaluated after a pre-selected amount of time. In some embodiments, the engrafted cells are monitored for cell survival. For example, the cell survival may be monitored via bioluminescence imaging (BLI), wherein the cells are transduced with a luciferase expression construct for monitoring cell survival. In some embodiments, the engrafted cells are visualized by immunostaining and imaging methods known in the art. In some embodiments, the engrafted cells express known biomarkers that may be detected to determine successful engraftment. For example, flow cytometry may be used to determine the surface expression of particular biomarkers. In some embodiments, the hypoimmunogenic cells are engrafted to the intended tissue site as expected (e.g., successful engraftment of the hypoimmunogenic cells). In some embodiments, the hypoimmunogenic cells are engrafted to the intended tissue site as needed, such as at a site of cellular deficiency. In some embodiments, the hypoimmunogenic cells are engrafted to the intended tissue site in the same manner as a non-engineered primary cell (e.g., a primary cell not comprising modification(s)) would be engrafted to the intended tissue site. In some embodiments, the hypoimmunogenic cells are assayed for function. In some embodiments, the hypoimmunogenic cells are assayed for function prior to their engraftment to the intended tissue site. In some embodiments, the hypoimmunogenic cells are assayed for function following engraftment to the intended tissue site. In some embodiments, the function of the hypoimmunogenic cells is evaluated after a pre-selected amount. In some embodiments, the function of the engrafted cells is evaluated by the ability of the cells to produce a detectable phenotype. For example, engrafted islet cells and/or beta islet cells function may be evaluated based on the restoration of lost glucose control due to diabetes. In some embodiments, the function of the hypoimmunogenic cells is as expected (e.g., successful function of the hypoimmunogenic cells while avoiding antibody-mediated rejection). In some embodiments, the function of the hypoimmunogenic cells is as needed, such as sufficient function at a site of cellular deficiency while avoiding antibody-mediated rejection. In some embodiments, the hypoimmunogenic cells function in the same manner as a non-engineered primary cell (e.g., a primary cell not comprising modification(s)) would function, while avoiding antibody-mediated rejection.

In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic cell growth (e.g. teratomas) that escape the host immune system. In some instances, hypoimmunogenic cell derivatives are transduced to express luciferase and can then followed using bioluminescence imaging. Similarly, the T cell and/or B cell response of the host animal to such cells are tested to confirm that the cells do not cause an immune reaction in the host animal. T cell responses can be assessed by ELISPOT, ELISA, FACS, PCR, or mass cytometry (CYTOF). B cell responses or antibody responses are assessed using FACS or Luminex. Additionally or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of hypoimmunogenic cells is determined using an allogeneic humanized immunodeficient mouse model. In some instances, the hypoimmunogenic cells are transplanted into an allogeneic humanized NSG-SGM3 mouse and assayed for cell rejection, cell survival, and teratoma formation. In some instances, grafted hypoimmunogenic cells display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety

As will be appreciated by those in the art, the successful reduction of the one or more MHC class I molecules function (HLA I when the cells are derived from human cells) in the pluripotent cells can be measured using techniques known in the art and as described below; for example, FACS techniques using labeled antibodies that bind the HLA complex; for example, using commercially available HLA-A, B, C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the one or more MHC class II molecules function (HLA II when the cells are derived from human cells) in the pluripotent cells or their derivatives can be measured using techniques known in the art such as Western blotting using antibodies to the protein, FACS techniques, RT-PCR techniques, etc.

In addition, the cells can be tested to confirm that the HLA II complex is not expressed on the cell surface. Again, this assay is done as is known in the art (See FIG. 21 of WO2018132783, for example) and generally is done using either Western Blots or FACS analysis based on commercial antibodies that bind to human HLA Class II molecules HLA-DR, DP and most DQ antigens.

In addition to the reduction of HLA I and II (or MHC class I molecules and class II molecules), the hypoimmunogenic cells provided herein have a reduced susceptibility to macrophage phagocytosis and NK cell killing. The resulting hypoimmunogenic cells “escape” the immune macrophage and innate pathways due to the expression of one or more CD24 transgenes.

### III. Modified Cell Compositions

Certain aspects of the present disclosure relate to compositions comprising any of the modified cells described herein, or populations of such modified cells. In some embodiments, the compositions are pharmaceutical compositions.

In some embodiments, the compositions provided herein further include a pharmaceutically acceptable excipient or carrier. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polysorbates (TWEEN™), poloxamers (PLURONICS™) or polyethylene glycol (PEG). In some embodiments, the pharmaceutical composition includes a pharmaceutically acceptable buffer (e.g., neutral buffer saline or phosphate buffered saline). In some embodiments, the pharmaceutical composition can contain one or more excipients for modifying, maintaining or preserving, for example, the pH, osmolarity, viscosity, clarity, color, isotonicity, odor, sterility, stability, rate of dissolution or release, adsorption, or penetration of the composition. In some aspects, a skilled artisan understands that a pharmaceutical composition containing cells may differ from a pharmaceutical composition containing a protein.

The pharmaceutical composition in some embodiments contains modified cells as described herein in amounts effective to treat or prevent a disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, compositions comprising modified cells as described herein are administered using standard administration techniques, formulations, and/or devices. Also provided are formulations and devices, such as syringes and vials, for storage and administration of the compositions. Compositions of the disclosure cells can be administered via localized injection, including catheter administration, systemic injection, localized injection, intravenous injection, or parenteral administration. When administering a therapeutic composition (e.g., a pharmaceutical composition containing a modified cell), it will generally be formulated in a unit dosage injectable form (solution, suspension, emulsion).

Formulations include those for intravenous, intraperitoneal, or subcutaneous, administration. In some embodiments, the cells of the disclosure are administered parenterally. The term “parenteral,” as used herein, includes intravenous, intramuscular, subcutaneous, rectal, vaginal, and intraperitoneal administration. In some embodiments, the cells of the disclosure are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, or dispersions, which may in some aspects be buffered to a selected pH. Liquid compositions are somewhat more convenient to administer, especially by injection. Liquid compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the cells in a solvent, such as in admixture with a suitable carrier, diluent, or excipient such as sterile water, physiological saline, glucose, dextrose, or the like.

In some embodiments, a pharmaceutically acceptable carrier can include all solvents, dispersion media, coatings, antibacterial and antifungal agents, isotonic and absorption delaying agents, and the like, compatible with pharmaceutical administration (Gennaro, 2000, Remington: The science and practice of pharmacy, Lippincott, Williams & Wilkins, Philadelphia, PA). Examples of such carriers or diluents include, but are not limited to, water, saline, Ringer's solutions, dextrose solution, and 5% human serum albumin. Liposomes and non-aqueous vehicles such as fixed oils may also be used. Supplementary active compounds can also be incorporated into the compositions. The pharmaceutical carrier should be one that is suitable for the modified cells, such as a saline solution, a dextrose solution or a solution comprising human serum albumin. In some embodiments, the pharmaceutically acceptable carrier or vehicle for such compositions is any non-toxic aqueous solution in which the modified cells can be maintained, or remain viable, for a time sufficient to allow administration of live cells. For example, the pharmaceutically acceptable carrier or vehicle can be a saline solution or buffered saline solution.

In some embodiments, the composition, including pharmaceutical composition, is sterile. In some embodiments, isolation, enrichment, or culturing of the cells is carried out in a closed or sterile environment, for example and for instance in a sterile culture bag, to minimize error, user handling and/or contamination. In some embodiments, sterility may be readily accomplished, e.g., by filtration through sterile filtration membranes. In some embodiments, culturing is carried out using a gas permeable culture vessel. In some embodiments, culturing is carried out using a bioreactor.

Also provided herein are compositions that are suitable for cryopreserving the provided modified cells. In some embodiments, the provided modified cells are cryopreserved in a cryopreservation medium. In some embodiments, the cryopreservation medium is a serum free cryopreservation medium. In some embodiments, the composition comprises a cryoprotectant. In some embodiments, the cryoprotectant is or comprises DMSO and/or glycerol. In some embodiments, the cryopreservation medium is between at or about 5% and at or about 10% DMSO (v/v). In some embodiments, the cryopreservation medium is at or about 5% DMSO (v/v). In some embodiments, the cryopreservation medium is at or about 6% DMSO (v/v). In some embodiments, the cryopreservation medium is at or about 7% DMSO (v/v). In some embodiments, the cryopreservation medium is at or about 7.5% DMSO (v/v). In some embodiments, the cryopreservation medium is at or about 8% DMSO (v/v). In some embodiments, the cryopreservation medium is at or about 9% DMSO (v/v). In some embodiments, the cryopreservation medium is at or about 10% DMSO (v/v). In some embodiments, the cryopreservation medium contains a commercially available cryopreservation solution (CryoStor™ CS10). CryoStor™ CS10 is a cryopreservation medium containing 10% dimethyl sulfoxide (DMSO). In some embodiments, compositions formulated for cryopreservation can be stored at low temperatures, such as ultra-low temperatures, for example, storage with temperature ranges from −40° C. to −150° C., such as or about 80° C.±6.0° C.

In some embodiments, the pharmaceutical composition comprises modified cells described herein and a pharmaceutically acceptable carrier comprising 31.25% (v/v) Plasma-Lyte A, 31.25% (v/v) of 5% dextrose/0.45% sodium chloride, 10% dextran 40 (LMD)/5% dextrose, 20% (v/v) of 25% human serum albumin (HSA), and 7.5% (v/v) dimethylsulfoxide (DMSO).

In some embodiments, the cryopreserved modified cells are prepared for administration by thawing. In some cases, the modified cells can be administered to a subject immediately after thawing. In such an embodiment, the composition is ready-to-use without any further processing. In other cases, the modified cells are further processed after thawing, such as by resuspension with a pharmaceutically acceptable carrier, incubation with an activating or stimulating agent, or are activated washed and resuspended in a pharmaceutically acceptable buffer prior to administration to a subject.

### IV. Method of Eliminating

The present disclosure provides methods for eliminating proliferating cells comprising providing a population of cells with an inducer, wherein the population of cells express a nucleic acid encoding a selection agent integrated at an endogenous proliferation gene locus and operably linked to the promoter of the endogenous proliferation gene, and wherein providing the inducer causes elimination of proliferating cells.

In some embodiments, the providing of the method provided herein is administering the inducer to an individual harboring the population of cells. In some embodiments, the method provided herein further comprises administering the population of cells to a subject either before or after the inducer is provided to the subject. In some embodiments, the population of cells is incubated with the inducer prior to administration to the subject. In some embodiments, the population of cells are administered to a subject before the inducer is provided to the subject. In some embodiments, the providing comprises incubating the population of cells in vitro. In some embodiments, the endogenous proliferation gene locus is selected from the group consisting of AURKB, CDC20, RRM2, CDK1, BIRC5, TOP2A, PTTG1, CCNB1, TPX2, KIF11, SPC25, CENPK, SMC4, TYMS, H2AZ1, TMSB15A, CENPF, and MKI67. In some embodiments, the endogenous proliferation gene locus is selected from the group consisting of AURKB, RRM2, TOP2A, PTTG1, CCNB1, SPC25, CENPK, SMC4, TYMS, H2AZ1, TMSB15A, CENPF, and MKI67. In some embodiments, the method selectively eliminates proliferating cells, cancer cells, pluripotent cells, multipotent stem cells, progenitor cells, de-differentiated cells, undifferentiated cells, and/or partially differentiated cells. In some embodiments, the population of cells comprises proliferating cells and non-proliferating cells. In some embodiments, the differentiated cells and/or non-proliferating cells are not eliminated.

The present disclosure provides methods for eliminating a specific cell type comprising providing a population of cells with an inducer, wherein the population of cells express a nucleic acid encoding an agent (e.g., a detection agent or selection agent) from an off-target cell marker gene locus and operably linked to the promoter of the off-target cell marker gene, and wherein providing the inducer causes elimination of an off-target cell.

In some embodiments, the providing of the method provided herein is administering the inducer to an individual harboring the population of cells. In some embodiments, the method provided herein further comprises administering the population of cells to a subject either before or after the inducer is provided to the subject. In some embodiments, the population of cells is incubated with the inducer prior to administration to the subject. In some embodiments, the population of cells are administered to a subject before the inducer is provided to the subject. In some embodiments, the providing comprises incubating the population of cells in vitro. In some embodiments, the off-target cell marker gene is selected from the group consisting of ANXA1, KRT19, CTSC, DSC2, ARHGAP29, KRT18, KRT8, CD9, PLK2, and KRT17. In some embodiments, the method provided herein selectively eliminates cancer cells, pluripotent cells, multipotent stem cells, progenitor cells, de-differentiated cells, undifferentiated cells, partially differentiated cells, and/or off-target cells. In some embodiments, the inducer does not cause elimination of a cell type other than the off-target cell. In some embodiments, the on-target cells are not eliminated. In some embodiments, the population of cells comprises on-target cells and off-target cells. In some embodiments, the population of cells comprises therapeutic cells and off-target cells. In some embodiments, the therapeutic cells are not eliminated. In some embodiments, the population of cells are stem cell derived cells or primary cells. In some embodiments, the stem cells are pluripotent stem cells, embryonic stem cells, induced pluripotent stem cells, multipotent stem cells, or adult stem cells. In some embodiments, the cells are autologous cells. In some embodiments, the cells are allogeneic cells. In some embodiments, the allogeneic cells are derived from one or more donors.

In some embodiments, the methods provided herein comprises administering the inducer is if cell proliferation is detected or if information indicating cell proliferation is obtained. In some embodiments, the inducer is administered if expression of proliferation markers is detected or if information indicating expression of proliferation markers is obtained. In some embodiments, the inducer is administered if an excess number of cells is detected or if information indicating an excess number of cells is obtained. In some embodiments, the method provided herein further comprises detecting proliferation using flow cytometry for a proliferation marker. In some embodiments, the method provided herein further comprises detecting proliferation using single-cell RNA-sequencing for expression of proliferation markers.

In some embodiments, the method provided herein eliminates at least 5%, at least 10%, at least 15%, at least 20%, at least 25%, at least 30%, at least 35%, at least 40%, at least 45%, at least 50%, at least 55%, at least 60%, at least 65%, at least 70%, or at least 75% of proliferating cells at day 5 of spontaneous differentiation. In some embodiments, the method provided herein eliminates at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of proliferating cells at day 18 of spontaneous differentiation. In some embodiments, the method provided herein eliminates 100% of proliferating cells at day 18 of spontaneous differentiation.

### V. Method of Detecting

The present disclosure provides methods of detecting a proliferating cell expressing a detection agent in a population of cells, wherein the population of cells comprises a nucleic acid encoding the detection agent integrated at an endogenous proliferation gene locus and operably linked to the promoter of the endogenous proliferation gene, and wherein expression of the nucleic acid encoding the detection agent is regulated by the promoter of the endogenous proliferation gene and/or regulatory elements of the endogenous proliferation gene.

The present disclosure provides methods of detecting a proliferating cell expressing a detection agent in a population of cells isolated from an individual, wherein the population of cells comprises a nucleic acid encoding the detection agent integrated at an endogenous proliferation gene locus and operably linked to the promoter of the endogenous proliferation gene, and wherein expression of the nucleic acid encoding the detection agent is regulated by the promoter of the endogenous proliferation gene and/or regulatory elements of the endogenous proliferation gene.

The present disclosure provides methods of detecting a proliferating cell expressing a detection agent in a population of cells in an individual, wherein the population of cells comprises a nucleic acid encoding the detection agent integrated at an endogenous proliferation gene locus and operably linked to the promoter of the endogenous proliferation gene, and wherein expression of the nucleic acid encoding the detection agent is regulated by the promoter of the endogenous proliferation gene and/or regulatory elements of the endogenous proliferation gene.

The present disclosure provides methods of detecting an off-target cell expressing a detection agent in a population of cells, wherein the population of cells comprises a nucleic acid encoding the detection agent integrated at an off-target cell marker gene locus and operably linked to the promoter of the off-target cell marker gene, and wherein expression of the nucleic acid encoding the detection agent is regulated by the promoter of the off-target cell marker gene and/or regulatory elements of the off-target cell marker gene.

The present disclosure provides methods of detecting an off-target cell expressing a detection agent in a population of cells isolated from an individual, wherein the population of cells comprises a nucleic acid encoding the detection agent integrated at an off-target cell marker gene locus and operably linked to the promoter of the off-target cell marker gene, and wherein expression of the nucleic acid encoding the detection agent is regulated by the promoter of the off-target cell marker gene and/or regulatory elements of the off-target cell marker gene.

The present disclosure provides methods of detecting an off-target cell expressing a detection agent in a population of cells in an individual, wherein the population of cells comprises a nucleic acid encoding the detection agent integrated at an off-target cell marker gene locus and operably linked to the promoter of the off-target cell marker gene, and wherein expression of the nucleic acid encoding the detection agent is regulated by the promoter of the off-target cell marker gene and/or regulatory elements of the off-target cell marker gene.

In some embodiments, the method provided herein further comprising removing or eliminating the proliferating cell or the off-target cell if expression of the detection agent is identified. In some embodiments, the method provided herein further comprises treating the population of cells with an agent that recognizes the detection agent, wherein treating the population of cells with the agent that recognizes the detection agent eliminates the proliferating cell or the off-target cells or targets the proliferating cell or the off-target cells for elimination. In some embodiments, the method provided herein further comprises surgically removing the proliferating cell or the off-target cell if expression of the detection agent is identified. In some embodiments, the method provided herein further comprises purifying the population of cells to select for cells not expressing the detection agent prior to administration to a patient if expression of the detection agent is identified. In some embodiments, the method provided herein further comprises modifying a treatment when the proliferating cell or the off-target cell expressing the detection agent is identified. In some embodiments, the method provided herein further comprises administering an additional therapy when the proliferating cell or the off-target cell expressing the detection agent is identified. In some embodiments, the method provided herein further comprises administering cells that are not expressing the detection agent to a subject. In some embodiments, a treatment regimen is not modified when there are no cells expressing the detection agent identified.

### A. Detection of Barcodes

In some embodiments, the method provided herein comprises detecting barcodes in a sample, wherein detection of a barcode indicates the presence of a transgene, including components in a vector comprising an identifying region comprising the barcode by which the barcode can be detected. In some embodiments, the method further comprises detecting a second barcode in the sample, wherein detection of the second barcode indicates the presence of a second transgene. In some embodiments, the method further comprises detecting a third barcode in the sample, wherein detection of the third barcode indicates the presence of a third transgene. In some embodiments, the first and/or the second barcodes are detected using sequencing and/or probe binding. In some embodiments, one or more probes bind to a barcode. In some embodiments, a probe binding site is a barcode. In some embodiments, the sample is a blood sample, plasma sample, or tissue sample. In some embodiments, the sample comprises a cell or population of cells, e.g., T cells, B cells, NK cells, islet cells such as beta cells, or hypoimmunogenic cells thereof.

i. Barcode Binding

In some embodiments, the identifying region comprises a barcode and primer binding sites. The primer binding sites flank the barcode region, thereby allowing for the amplification and/or detection of the barcode by any method known in the art. Amplification techniques can include a polymerase chain reaction (PCR) amplification technique or a non-PCR amplification technique, for example, an isothermal amplification technique. The primer binding sites can bind to nucleic acid primers that comprise sequences complementary to the primer bind site sequences. In some embodiments, the primers are about 10 to about 50 nucleotides in length, for example about any of 10, 11, 12, 13, 14, 15, 16, 17, 18, 19, 20, 21, 22, 23, 24, 25, 30, 35, 40, 45, or 50 nucleotides in length. In some embodiments, the primers are about 5-15, about 10-20, about 15-25, about 20-30, about 25-35, about 30-40, about 35-45, about 40-50, about 45-55 nucleotides in length. In some instances, the primer sequences may be longer than 50 nucleotides, for example about 50, about 60, about 70, about 80, about 90, or more nucleotides in length. In some embodiments, the primer sequences are about 10 to about 30 nucleotides in length.

In some embodiments, the first identifying region and/or the second identifying region comprises one or more probe binding sites. Probe binding sites are nucleic acid sequences to which specific probes recognize and hybridize. In some embodiments, the probes bind to specific nucleic acid sequences. In some embodiments, the probes bind to a barcode. In some embodiments, the probes comprise or are nucleic acid molecules, polypeptides, or chemical molecules. Antibodies can include primary or secondary antibodies, wherein secondary antibodies can bind to primary antibodies to amplify the probe binding signal. Nucleic acid molecules can comprise either RNA or DNA molecules that are complementary to the probe binding sites. In some instances, the probes (e.g., the nucleic acid molecules) can be further attached to a marker for detection as described below. In some embodiments, the probes are attached, e.g., conjugated, to a detection marker. In some embodiments, the detection molecule can include any one or more of fluorophores, biotin, enzymes, radioisotopes, non-radioactive heavy metal isotopes, etc. Additional examples of probes include, but are not limited to, two-photon fluorescent probes, Raman probes (based on alkyne or nitrile tags), Siderophore-Dioxetane Probes, or enzyme-activated fluorescent probes.

Known fluorophores include, for example, any of Brilliant Ultra Violet 395, Alexa Fluor 350, Brilliant Ultra Violet 496, Qdot 525 Probe, Brilliant Ultra Violet 563, Qdot 565 Probe, Qdot 605 Probe, Brilliant Ultra Violet 615, Qdot 655 Probe, Brilliant Ultra Violet 661, Qdot 705 Probe, Brilliant Ultra Violet 737, Qdot 800 Probe, Brilliant Ultra Violet 805, Alexa Fluor 405, Brilliant Violet 421, Super Bright 436, eFluor 450, Pacific Blue, Coumarin and Coumarin Derivatives, Brilliant Violet 480, Cyan Fluorescent Protein (CFP), eFluor 506, Pacific Green, Pacific Orange, Super Bright 600, Super Bright 645, Brilliant Violet 650, Super Bright 702, Brilliant Violet 711, Super Bright 780, Brilliant Violet 786, Green Fluorescent Protein (GFP), BODIPY FL, NovaFluor Blue 510, Fluorescein (FITC), Alexa Fluor 488, Oregon Green 488, NovaFluor Blue 530, NovaFluor Blue 555, NovaFluor Blue 585, NovaFluor Blue 610-30S, NovaFluor Blue 610-70S, NovaFluor Blue 660-40S, NovaFluor Blue 660-120S, PerCP-Cyanine5.5, PerCP-eFluor 710, Alexa Fluor 532, Cy3, NovaFluor Yellow 570, Alexa Fluor 555, Alexa Fluor 561, Alexa Fluor 546, R-phycoerythrin (R-PE), Tetramethylrhodamine (TRITC), Red Fluorescent Protein (RFP), NovaFluor Yellow 590, Alexa Fluor 568, PE-eFluor 610, Texas Red (and Texas Red-X), NovaFluor Yellow 610, Alexa Fluor 594, NovaFluor Yellow 660, NovaFluor Yellow 690, NovaFluor Yellow 700, NovaFluor Yellow 730, NovaFluor Yellow 755, PE-Cyanine7, NovaFluor Red 660, Allophycocyanin (APC), Cy5, eFluor 660, Alexa Fluor 647, NovaFluor Red 685, NovaFluor Blue 690, Alexa Fluor 660, NovaFluor Red 700, Alexa Fluor 680, NovaFluor Red 710, Alexa Fluor 700, NovaFluor Red 725, NovaFluor Red 755, Alexa Fluor 750, APC-eFluor 780, FAM, HEX, Rhodamine Red-X, Tamara, YY, Atto 550, Atto 590, Atto 700, Rox, TruRed, Cy7, Red 613, Cy3.5 581, Cy5.5, DAPI, Hoechst, SYTOX blue, SYTOX green, SYTOX orange, YOYO-1, TOTO-1, TO-PRO-1, chromomycin A3, mithramycin, propidium iodide, ethidium bromide, SYBR Green, any KIRAVIA dyes (e.g., KIRVIA Blue 520), PE-Dazzle 594, PE-Fire 640, PE-Cy5, PE-Fire 700, PE-FIRE 810, PerCP, APC-Cyanine 7, APC-Fire 750, APC-Fire 810, Spark UV 387, Spark Violet 423, Spark Violet 500, Spark Violet 538, Spark Blue 550, Spark Blue 574, Spark YG 570, Spark YG 581, Spark YG 593, Spark NIR 685, Spark Red 718, Brilliant Violet 510, Brilliant Violet 570, Brilliant Violet 605, Brilliant Violet 750, 7-AAD, APC-H7, Apotracker Green, APC-R700, Brilliant Blue 515, Brilliant Blue 700, Calcein-AM, Calcein Red-AM, Calcein Violet-AM, CF 570, CytoPhase Violet, DRAQ5, DRAQ7, Helix NP Blue, Helix NP Green, Helix NP NIR, MitoSpy Green, MitoSpy NIR, MitoSpy Orange, MitoSpy Red, Tag-it Violet, VioBright FITC, Zombie Aqua, Zombie Green, Zombie NIR, Zombie Red, Zombie UV, Zombie Violet, and Zombie Yellow.

In some embodiments, the first probe and the second probe are each labeled with a distinct fluorophore. In some embodiments, the first probe and the second probe are used to detect the first barcode and the second barcode simultaneously or concurrently. In some embodiments, the first probe and the second probe are used to detect the first barcode and the second barcode separately. In some embodiments, the two or more distinct fluorophores conjugated to the two or more probes used to detect two or more barcodes simultaneously or concurrently do not spectrally overlap. Spectral overlap occurs when two fluorophores share similar excitation and emission wavelengths, such that the excitation spectra and the emission spectra are not readily distinguishable by the instruments used in the art to detect the presence of fluorophores. In some embodiments, the emission spectra of two or more fluorophores do not overlap. In some embodiments, the excitation spectra of two or more fluorophores do not overlap. In some embodiments, neither the excitation spectra nor the emission spectra of two or more fluorophores overlap. In some embodiments, the first probe and/or the second probe is labeled with FAM and/or HEX.

Known radioisotopes (i.e., radioactive isotope, radionuclide, or radioactive nuclide) include, for example, Phosphorus-32, Hydrogen-3 (tritium), Carbon-14, Chlorine-36, Lead-210, Chromium-51, Manganese-54, Cobalt-60, Zinc-65, Technetium-99, Cesium-137, Ytterbium-169, Iridium-192, Gold-198, Americium-241, Molybdenum-99, Iodine-123, Iodine-131, Samarium-153, Lutetium-177, Carbon-11, Nitrogen-13, Oxygen-15, Fluorine-18, Copper-64, Gallium-67, Thallium-201, Hydrogen-1, Phosphorus-31, Fluorine-19, Sodium-23, Carbon-13, Oxygen-17, Nitrogen-15, 2D FBP, 3DRP, OSEM2D, and OSEM3D/MAP. Non-radioactive heavy metal isotopes include 38 isotopes of lanthanides, 2 isotopes of indium, 1 isotope of yttrium, 6 isotopes of palladium, 1 isotope of bismuth (see, e.g., Han et al., Nat Protoc. 2018, 13(10):2121-2148), for example (but not limited to): 89Y, 113In, 115In, 139La, 140Ce, 141Pr, 142Nd, 143Nd, 144Nd, 145Nd, 146Nd, 147Sm, 148Nd, 149Sm, 150Nd, 151Eu, 152Sm, 153Eu, 154Sm, 155Gd, 156Gd, 157Gd, 158Gd, 159Tb, 160Gd, 161Dy, 162Dy, 163Dy, 164Dy, 165Ho, 166Er, 167Er, 168Er, 169Tm, 170Er, 171Yb, 172Yb, 173Yb, 174Yb, 175Lu, 176Yb, 1911r, 193Ir, 195Pt, and 209Bi. Known reporter enzymes include, for example, horseradish peroxidase (HRP), alkaline phosphatase (AP), glucose oxidase, 0-galactosidase, 0-glucuronidase (GUS), luciferase, and Chloramphenicol O-Acetyltransferase (CAT).

Florescent molecules can be identified by multiple technologies including, for example, fluorescence microscopy, flow cytometry, spectral flow cytometry, microplate reader, immunohistochemistry, multiplexed immunofluorescence (e.g. PhenoCycler, Vectra Polaris), fluorescence spectroscopy, fluorescence in situ hybridization, PET/fluorescence imaging (e.g., CRi Maestro, IVIS Lumina, IVIS Spectrum, iThera MSOT), etc. Radioisotopes can be detected by, e.g., single-photon emission computed tomography (SPECT), positron emission tomography (PET), magnetic resonance imaging, scintillation detector/counter, laser spectroscopy, isotope ratio mass spectrometry, or autoradiography. Non-radioactive heavy metal isotopes can be detected by methods including cytometry by time of flight (CyTOF), mass spectrometry (e.g., LC-MS), reversed-phase high-performance liquid chromatography (RP-HPLC), etc.

In some embodiments, the probe is between 10 and 50 nucleotides in length. In some embodiments, the probe is at least 10, 15, 20, 25, 30, 35, 40, 45, or 50 nucleotides in length. In some embodiments, the probe is about 10, 15, 20, 25, 30, 35, 40, 45, or 50 nucleotides in length. In some embodiments, the probe is between 10 and 50, 15 and 45, 20 and 40, or 25 and 35 nucleotides in length. In some embodiments, the probe is about any of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or nucleotides in length. In some embodiments, the probe is between 18 and 30 nucleotide in length. In some embodiments, the probe binds to a probe binding site that comprises a sequence that is complementary to the barcode. In some embodiments, the probe and the barcode comprise a sequence of the same length. In some embodiments, the probe and the barcode comprise sequences of different lengths. In some embodiments, the probe binding site comprises a sequence that is longer than the sequence comprised by the barcode. In some embodiments, the probe binding site comprises a sequence that is shorter than the sequence comprised by the barcode.

In some embodiments, the method comprises the detection of a first probe binding site and/or a second probe binding site with a first complementary probe and/or a second complementary probe. In some embodiments, the barcode is randomly generated, and the probe binding site comprises the nucleic acid sequence of SEQ ID NO:31, wherein NNNNNNNN is the barcode and wherein N represents a nucleotide selected from the group consisting of A, T, C, and G. For example, in some embodiments, the method comprises detection of a barcode using a probe that binds to the probe binding site comprising the nucleotide sequence set forth in any one of SEQ ID NOs:18-23 or a variant thereof comprising about 1, 2, 3, 4, or 5 nucleotide substitutions, insertions, or deletions.

In some embodiments, the probe binds directly to the barcode. In some embodiments, the probe comprises a sequence that is complementarity to the barcode. In some embodiments, the probe and the barcode share between about 70% and 100% complementarity. In some embodiments, the probe and the barcode sequence share at least about any of 70%, 80%, 85%, 90%, 92%, 95%, 96%, 97%, 98%, 99%, 99%, or 100% complementarity.

In some embodiments, the probe sequence partially or completely overlaps with a barcode. In some embodiments, the probe sequence and the barcode partially overlap within the 5′ region and/or 3′ region of the barcode. In some embodiments, the probe sequence and the barcode partially overlap within the 5′ region of the barcode. In some embodiments, the probe sequence and the barcode partially overlap within the 3′ region of the barcode. In some embodiments, the probe sequence and the barcode partially overlap within the 5′ region and the 3′ region of the barcode.

In some embodiments, one or more probes is incubated with a sample comprising one or more cells, wherein the cells comprise one or more vectors encoding one or more transgenes and identifying regions comprising one or more barcodes for about any of 5, 10, 15, 20, 35, 30, 45, 60, 90, or 120 minutes. In some embodiments, the cells are intact. In some embodiments, the cells are fixed. In some embodiments, the cells are lysed. In some embodiments, one or more probes is incubated with the sample for about any of 1, 2, 3, 4, 5, 6, 7, 8, 9, or 10 hours. In some embodiments, probes incubated with the sample are then detected by a method known in the art that is appropriate for the detection marker, as described herein.

ii. Detection via Sequencing

In some embodiments, the first barcode and/or the second barcode are detected using sequencing and/or probe binding, wherein sequencing comprises one or more of Sanger sequencing, next-generation sequencing (NGS), shotgun sequencing, single molecule real time (SMRT) sequencing, nanopore DNA sequencing, massively parallel signature sequencing (MPSS), polony sequencing, 454 pyrosequencing, Illumina (Solexa) sequencing, combinatorial probe anchor synthesis (cPAS), SOLiD sequencing, Ion Torrent semiconductor sequencing, DNA nanoball sequencing, helioscope single molecule sequencing, and microfluidic systems.

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, targeted sequencing or direct sequencing. In some instances, sequencing may be performed using, e.g., Sanger sequencing. In some instances, the sequencing may comprise whole genome sequencing or whole exome sequence wherein detection of, e.g., genomic rearrangements, repetitive sequence elements, gene fusions, and novel transcripts within a cell is also desired.

The disclosed methods may be implemented using sequencing platforms such as the Roche 454, Illumina Solexa, ABI-SOLiD, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target loci in nucleic acids extracted from a sample, such as a population of cells as described herein, are described in more detail in, e.g., International Patent Application Publication No. WO 2020/236941, the entire content of which is incorporated herein by reference.

Quantitative polymerase chain reaction (qPCR) or real-time PCR (RT-PCR) monitors the amplification of a targeted DNA molecule during the PCR reaction. Detection of PCR products in real-time may use non-specific fluorescent dyes that intercalate with any double-stranded DNA or sequence-specific DNA probes consisting of oligonucleotides that are labeled with a fluorescent reporter. This technology permits detection only after hybridization of the probe with its complementary nucleic acid sequence. DNA-binding dyes known to one of ordinary skill in the art include, but is not limited to, SYBR Green, DAPI, propidium iodide, Hoechst, ethidium bromide, mithramycin, chromomycin A3, etc. Oligonucleotides labeled with fluorescent reporters known to one of ordinary skill in the art include, but are not limited to, hydrolysis probes, molecular beacons, dual hybridization probes, eclipse probes, UniPrimer, Scorpions primers, LUX primers, and QZyme primers.

Digital droplet PCR (ddPCR) is a digital PCR method utilizing a water-oil emulsion droplet system to precisely measure absolute quantities of nucleic acid molecules. ddPCR reactions are prepared with reporter fluorophores, such as FAM and HEX, or and intercalating dye. Droplets are formed in a water-oil emulsion to form massive partitions that separate the template DNA molecules. ddPCR fractionates a sample into 20,000 nanoliter-sized droplets, and PCR amplification of the template molecules occurs in each individual droplet using a thermal cycler. Partitioning enables the measurement of thousands of independent amplification events within a single sample. The droplets are streamed in a single file on a reader, which counts the fluorescent positive and negative droplets to calculate the DNA concentration. Fluorescence in two channels is measured for the individual droplets. Positive droplets containing at least one copy of the target DNA molecule will exhibit increased fluorescence, compared to negative droplets that do not contain the target DNA molecule. ddPCR data is shown as a plot as fluorescence intensity versus droplet number. Positive droplets are scored as positive, assigned a value of 1, and appear above a threshold line in the graph while negative droplets are scored as negative, assigned a value of 0, and appear below the threshold line. ddPCR is known to one of ordinary skill in the art. See, e.g., Hindson et al. (2011), Anal Chem. 83(22):8604-10; and Pinheiro et al. (2012), Anal Chem. 84(2):1003-11.

In some embodiments, the method further comprises contacting the sample with i) a first probe, ii) a second probe, or iii) a first probe and a second probe, wherein the first probe has a different sequence from the second probe; and detecting binding of i) the first probe to a first barcode in the first vector, ii) the second probe to a second barcode in the second vector, or iii) the first probe to a first barcode in a first vector and the second probe to a second barcode in a second vector. In some embodiments, the first probe and the second probe comprise the same sequence. In some embodiments, the first probe and the second probe comprise different sequences. In some embodiments, the method further comprises contacting the sample with a third probe, wherein the third probe has a different sequence from the first probe or the second probe; and detecting the binding of the third probe to the third barcode in a third vector.

In some the sample comprising one or more cells is screened and/or monitored using the methods provided herein. In some embodiments, the sample is screened and/or monitored for the presence of a transgene, e.g., by the detection of an associated barcode. In some embodiments, the sample is screened and/or monitored for the presence of two or more transgenes. In some embodiments, detection of the first barcode and the second barcode indicates the presence of i) a first vector encoding a first transgene and a second vector encoding a second transgene or ii) a first vector encoding a first transgene and a second transgene. In some embodiments, the detection of two or more barcodes is used to screen a sample comprising one or more cells (or one or more populations of cells), such as any cell or population of cells described in the present disclosure, for the presence of two or more barcodes. In some embodiments, the detection of two or more unique barcodes is used to screen a sample comprising one or more cells for the presence of two or more transgenes associated with the expression of the two or more barcodes.

### VI. Method of Making

Also provided herein are methods for generating a modified cell of the present disclosure.

### A. Insertion of Kill Switch(es)

In some aspects, provided herein are methods of generating a modified cell comprising one or more exogenous sequences, such as a kill switch, inserted into an endogenous proliferation gene or off-target cell marker gene, such as any of the endogenous proliferation genes or off-target cell marker genes of the disclosure. In some embodiments, the one or more exogenous sequences, such as a kill switch, are inserted into an endogenous proliferation gene or an off-target cell marker gene of a cell by HDR-mediated insertion using any of the gRNAs provided herein, such as a Mad7 gRNA provided herein. In some embodiments, the one or more exogenous sequences, such as a kill switch, are inserted into an endogenous proliferation gene or an off-target cell marker gene of a cell using a Mad7 nuclease and/or any of the gRNAs provided herein, such as a Mad7 gRNA provided herein.

In some embodiments, the kill switch is any of the kill switches described herein, for example, in Section IIC. In some embodiments, the kill switch comprises one or more recombination or cassette exchange sequences, such as any of the recombination or cassette exchange sequences described in Section IIC. In some embodiments, the site-specific recombinase includes any of the recombinases described in Section IIC. In some embodiments, the modified cell comprises any one of the cells described in Section III above.

Insertion of kill switch(es) into cells as described herein may require transfection or transduction of the relevant genome editing components, for example the components for nucleic acid-guided nuclease editing. Standard cell transfection and transduction methods are well known by those skilled in the art and can include any known method of transfection or transduction of a cell or population of cells, for example a mammalian cell, such as any of the methods included in Section VI. Furthermore, methods of genome insertion are well known in the art and described in more detail in Section II. In some embodiments, the modified cell may be transduced or transfected with separate editing and enzyme vectors. In some embodiments, the enzyme vectors comprise a nuclease, such as any selected from Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, TnpB, MAD2, Mad7, MAD2007, and other MADzymes and MADzyme systems (see U.S. Pat. Nos. 9,982,279; 10,337,028; 10,435,714; 10,011,849; 10,626,416; 10,604,746; 10,665,114; 10,640,754; 10,876,102; 10,883,077; 10,704,033; 10,745,678; 10,724,021; 10,767,169; and 10,870,761 for sequences and other details related to engineered and naturally-occurring MADzymes). In some embodiments, the modified cell described herein may already express the nuclease (e.g., the genome modifying complex may have already been transfected into the modified cell described herein or the coding sequence for the nuclease may be stably integrated into the cellular genome). In some embodiments, the modified cell described herein may be transduced or transfected with a single vector comprising all components required to perform nucleic acid-guided nuclease genome editing.

After the modified cell or population of such cells described herein is transduced or transfected with the components for genome editing, such as the components described above, the modified cell or population of such cells are cultured under conditions to promote genome editing and cell expansion. For example, in some embodiments, constitutive promoters are used to drive transcription of the nuclease and/or gRNA, and as a result, the modified cell or population of such cells can be grown in standard culture conditions for those cells. In other embodiments, inducible promoters are used such that the modified cell or population of such cells must be grown in inducing conditions in order to undergo genome editing.

In some embodiments, the methods provided herein comprise inserting at the endogenous proliferation gene or off-target cell marker gene one or more kills switches (e.g., any of the kill switches described in Section TIC herein).

In some embodiments, the methods provided herein further comprises inserting a nucleic acid encoding one or more kill switches at the endogenous proliferation gene or off-target cell marker gene. Methods for inserting sequences into an endogenous proliferation gene or off-target cell marker gene are described in detail in Section IIC(2) herein, and are further in detail described in Section VIIA, below.

In some embodiments, the nucleic acid is inserted into the endogenous proliferation gene or off-target cell marker gene by one or more gene edits. In some embodiments, the cell comprises a genome editing complex. In some embodiments, the method further comprises introducing into the cell a genome editing complex. Genome modifying complexes are described in detail in Section IIC(2) herein. In some embodiments, the genome editing complex comprises a genome targeting entity and a genome modifying entity. In some embodiments, the genome targeting entity localizes the genome editing complex to the s endogenous proliferation gene or off-target cell marker gene, optionally wherein the genome targeting entity is a nucleic acid-guided targeting entity.

In some embodiments, the genome targeting entity is selected from a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising a gRNA and a Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof. In some embodiments, the genome targeting entity is selected from Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.

In some embodiments, the genome modifying entity cleaves, deaminates, nicks, polymerizes, interrogates, integrates, cuts, unwinds, breaks, alters, methylates, demethylates, or otherwise destabilizes the target locus. In some embodiments, the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof. In some embodiments, the genome modifying entity is selected from a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof. In some embodiments, the genome modifying entity is selected from Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor, a target-primed reverse transcription (TPRT) editor, APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.

In some embodiments, the genome targeting entity and the genome modifying entity are different domains of a single polypeptide. In some embodiments, the genome targeting entity and genome modifying entity are two different polypeptides that are operably linked together. In some embodiments, the genome targeting entity and genome modifying entity are two different polypeptides that are not linked together.

In some embodiments, the genome editing complex comprises a guide nucleic acid having a targeting domain that is complementary to at least one sequence within the endogenous proliferation gene or off-target cell marker gene, optionally wherein the guide nucleic acid is a guide RNA (gRNA). In some embodiments, the gRNA can be selected from any of the gRNAs described in Section IIA above.

In some embodiments, the genome editing complex is an RNA-guided nuclease. In some embodiments, the RNA-guided nuclease comprises a Cas nuclease and a guide RNA (CRISPR-Cas combination). In some embodiments, the CRISPR-Cas combination is a ribonucleoprotein (RNP) complex comprising the gRNA and the Cas nuclease. In some embodiments, the Cas nuclease is a Type II or Type V Cas protein. In some embodiments, the Cas nuclease is selected from Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7.

In some embodiments, there is provided a method of making a cell comprising a selection agent or a detection agent integrated at an endogenous proliferation gene locus and operably linked to the promoter of the endogenous proliferation gene, the method comprising introducing a nucleic acid encoding a selection agent or a detection agent into the cell, wherein the nucleic acid encoding the selection agent or the detection agent is integrated in the genome of the cell at the endogenous proliferation gene locus.

In some embodiments, there is provided a method of making a cell comprising a selection agent or a detection agent integrated at an off-target cell marker gene locus and operably linked to the promoter of the off-target cell marker gene, the method comprising introducing a nucleic acid encoding a selection agent or a detection agent into the cell, wherein the nucleic acid encoding the selection agent or the detection agent is integrated in the genome of the cell at the off-target cell marker gene locus.

In some embodiments, the method provided herein comprise an integration of a nucleic acid, wherein the integration of a nucleic acid comprises a gene knock-in. In some embodiments, the integration of a nucleic acid comprises a gene knock-out. In some embodiments, the integration comprises a targeted integration method.

In some embodiments, the method provided herein comprises a genome editing complex. In some embodiments, the genome editing complex is encoded by one or more transgenes. In some embodiments, the genome editing complex comprises a genome targeting entity and a genome modifying entity. In some embodiments, the genome targeting entity is a nucleic acid-guided targeting entity. In some embodiments, the genome targeting entity is selected from the group consisting of: a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising a gRNA and a Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), and a Type II or Type V Cas protein, or functional portions thereof. In some embodiments, the genome targeting entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, and dCas13b, or functional portions thereof. In some embodiments, the genome modifying entity cleaves, deaminates, nicks, polymerizes, interrogates, integrates, cuts, unwinds, breaks, alters, methylates, demethylates, or otherwise destabilizes the target locus. In some embodiments, the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof. In some embodiments, the genome modifying entity is selected from the group consisting of: a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, an endonuclease-deficient-Cas protein, an enzymatically inactive Cas protein, a CRISPR-associated transposase (CAST), a Type II or Type V Cas protein, base editing, prime editing, and a Programmable Addition via Site-specific Targeting Elements (PASTE), or functional portions thereof. In some embodiments, the genome modifying entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor, a target-primed reverse transcription (TPRT) editor, APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, and a transcriptional repressor, or functional portions thereof. In some embodiments, the genome targeting entity and the genome modifying entity are different domains of a single polypeptide. In some embodiments, the genome targeting entity and genome modifying entity are two different polypeptides that are operably linked together. In some embodiments, the genome targeting entity and genome modifying entity are two different polypeptides that are not linked together. In some embodiments, the genome editing complex comprises a guide nucleic acid having a targeting domain that is complementary to at least one target locus, optionally wherein the guide nucleic acid is a guide RNA (gRNA). In some embodiments, the genome editing complex is an RNA-guided nuclease. In some embodiments, the RNA-guided nuclease comprises a Cas nuclease and a guide RNA (CRISPR-Cas combination). In some embodiments, the CRISPR-Cas combination is a ribonucleoprotein (RNP) complex comprising the gRNA and the Cas nuclease. In some embodiments, the Cas nuclease is a Type II or Type V Cas protein. In some embodiments, the Cas nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. In some embodiments, the targeted integration method is a Cas-directed homology-directed repair (HDR).

In some embodiments, the methods described herein comprise use of an endogenous proliferation gene. In some embodiments, the endogenous proliferation gene is selected from the group consisting of AURKB, CDK1, CDC20, RRM2, BIRC5, TOP2A, PTTG1, CCNB1, TPX2, KIF11, SPC25, CENPK, SMC4, TYMS, H2AZ1, TMSB15A, CENPF, and MKI67. In some embodiments, the endogenous proliferation gene is selected from the group consisting of AURKB, RRM2, TOP2A, PTTG1, CCNB1, SPC25, CENPK, SMC4, TYMS, H2AZ1, TMSB15A, CENPF, and MKI67. In some embodiments, the nucleic acid encoding the selection agent or the detection agent is integrated in the exon or intron of the endogenous proliferation gene. In some embodiments, the nucleic acid encoding the selection agent or the detection agent is integrated in the 5′ UTR or the 3′ UTR of the endogenous proliferation gene. In some embodiments, expression of the nucleic acid encoding the selection agent or a detection agent is regulated by the endogenous promoter and/or regulatory elements of the endogenous proliferation gene. In some embodiments, following integration, the endogenous proliferation gene locus comprises i) nucleic acid encoding the proliferation gene, ii) an internal ribosomal entry site or a self-cleaving RNA site, and iii) the nucleic acid encoding the selection agent or a detection agent. In some embodiments, (i) the endogenous proliferation gene is AURKB and the selection agent is iCaspase9; (ii) the endogenous proliferation gene is CDK1 and the selection agent is iCaspase9; (iii) the endogenous proliferation gene is CDC20 and the selection agent is iCaspase9; (iv) the endogenous proliferation gene is RRM2 and the selection agent is iCaspase9; (v) the endogenous proliferation gene is BIRC5 and the selection agent is iCaspase9; (vi) the endogenous proliferation gene is TOP2A and the selection agent is iCaspase9; (vii) the endogenous proliferation gene is PTTG1 and the selection agent is iCaspase9; (viii) the endogenous proliferation gene is CCNB1 and the selection agent is iCaspase9; (ix) the endogenous proliferation gene is TPX2 and the selection agent is iCaspase9; (x) the endogenous proliferation gene is KIF11 and the selection agent is iCaspase9; (xi) the endogenous proliferation gene is SPC25 and the selection agent is iCaspase9; (xii) the endogenous proliferation gene is CENPK and the selection agent is iCaspase9; (xiii) the endogenous proliferation gene is SMC4 and the selection agent is iCaspase9; (xiv) the endogenous proliferation gene is TYMS and the selection agent is iCaspase9; (xv) the endogenous proliferation gene is H2AZ1 and the selection agent is iCaspase9; (xvi) the endogenous proliferation gene is TMSB15A and the selection agent is iCaspase9; (xvii) the endogenous proliferation gene is CENPF and the selection agent is iCaspase9; (xviii) the endogenous proliferation gene is MKI67 and the selection agent is iCaspase9; (xix) the endogenous proliferation gene is AURKB and the selection agent is cytosine deaminase; (xx) the endogenous proliferation gene is CDK1 and the selection agent is cytosine deaminase; (xxi) the endogenous proliferation gene is CDC20 and the selection agent is cytosine deaminase; (xxii) the endogenous proliferation gene is RRM2 and the selection agent is cytosine deaminase; (xxiii) the endogenous proliferation gene is BIRC5 and the selection agent is cytosine deaminase; (xxiv) the endogenous proliferation gene is TOP2A and the selection agent is cytosine deaminase; (xxv) the endogenous proliferation gene is PTTG1 and the selection agent is cytosine deaminase; (xxvi) the endogenous proliferation gene is CCNB1 and the selection agent is cytosine deaminase; (xxvii) the endogenous proliferation gene is TPX2 and the selection agent is cytosine deaminase; (xxviii) the endogenous proliferation gene is KIF11 and the selection agent is cytosine deaminase; (xxix) the endogenous proliferation gene is SPC25 and the selection agent is cytosine deaminase; (xxx) the endogenous proliferation gene is CENPK and the selection agent is cytosine deaminase; (xxxi) the endogenous proliferation gene is SMC4 and the selection agent is cytosine deaminase; (xxxii) the endogenous proliferation gene is TYMS and the selection agent is cytosine deaminase; (xxxiii) the endogenous proliferation gene is H2AZ1 and the selection agent is cytosine deaminase; (xxxiv) the endogenous proliferation gene is TMSB15A and the selection agent is cytosine deaminase; (xxxv) the endogenous proliferation gene is CENPF and the selection agent is cytosine deaminase; or (xxxvi) the endogenous proliferation gene is MKI67 and the selection agent is cytosine deaminase.

In some embodiments, the selection agent or the detection agent of the methods described herein is expressed in proliferating cells. In some embodiments, the selection agent or the detection agent of the cell described herein is not expressed in non-proliferating cells. In some embodiments, the selection agent or the detection agent of the cell described herein is expressed in partially differentiated cells. In some embodiments, the selection agent or the detection agent of the cell described herein is expressed in an intermediate cell type made during differentiation. In some embodiments, the selection agent or the detection agent of the cell described herein is not expressed in differentiated cells. In some embodiments, the selection agent or the detection agent of the cell described herein is not expressed in a therapeutic cell. In some embodiments, the cell is selected from the group consisting of a pancreatic islet cell, an alpha cell, a beta cell, a gamma cell, a delta cell, an epsilon cell, a T cell, a neuron, a glial cell, a cardiomyocyte, a retinal pigmented epithelial cell, a hematopoietic progenitor cell, a natural killer cell, an endothelial cell, and a lung cell.

In some embodiments, the methods described herein comprise use of an off-target cell marker gene. In some embodiments, the off-target cell marker gene is selected from the group consisting of a pluripotency cell marker gene, a tumorigenic cell marker gene, a ductal marker gene, an enterochromaffin marker gene, a neural marker gene, acinar marker gene, intestinal marker gene, endothelial marker gene, mesenchymal fibroblast marker gene, muscle marker gene, osteoblast marker gene, and stromal marker gene. In some embodiments, the off-target cell marker gene is selected from the group consisting of ANXA1, KRT19, CTSC, DSC2, ARHGAP29, KRT18, KRT8, CD9, PLK2, and KRT17. In some embodiments, the selection agent is iCaspase 9 or cytosine deaminase. In some embodiments, (i) the off-target cell marker gene is ANXA1 and the selection agent is iCaspase9; (ii) the off-target cell marker gene is KRT19 and the selection agent is iCaspase9; (iii) the off-target cell marker gene is CTSC and the selection agent is iCaspase9; (iv) the off-target cell marker gene is DSC2 and the selection agent is iCaspase9; (v) the off-target cell marker gene is ARHGAP29 and the selection agent is iCaspase9; (vi) the off-target cell marker gene is KRT18 and the selection agent is iCaspase9; (vii) the off-target cell marker gene is KRT8 and the selection agent is iCaspase9; (viii) the off-target cell marker gene is CD9 and the selection agent is iCaspase9; (ix) the off-target cell marker gene is PLK2 and the selection agent is iCaspase9; (x) the off-target cell marker gene is KRT17 and the selection agent is iCaspase9; (xi) the off-target cell marker gene is ANXA1 and the selection agent is cytosine deaminase; (xii) the off-target cell marker gene is KRT19 and the selection agent is cytosine deaminase; (xiii) the off-target cell marker gene is CTSC and the selection agent is cytosine deaminase; (xiv) the off-target cell marker gene is DSC2 and the selection agent is cytosine deaminase; (xv) the off-target cell marker gene is ARHGAP29 and the selection agent is cytosine deaminase; (xvi) the off-target cell marker gene is KRT18 and the selection agent is cytosine deaminase; (xvii) the off-target cell marker gene is KRT8 and the selection agent is cytosine deaminase; (xviii) the off-target cell marker gene is CD9 and the selection agent is cytosine deaminase; (xix) the off-target cell marker gene is PLK2 and the selection agent is cytosine deaminase; or (xx) the off-target cell marker gene is KRT17 and the selection agent is cytosine deaminase. In some embodiments, the off-target cell marker gene is not a beta cell marker gene, a T cell marker gene, a neuronal cell marker gene, a glial cell marker gene, a cardiac cell marker gene, a retinal pigment epithelium (RPE) cell marker gene, a hematopoietic progenitor cell marker gene, a natural killer cell marker gene, an endothelial cell marker gene, or a lung cell marker gene.

In some embodiments, the methods described herein comprise providing an inducer, wherein the inducer is selected from the group consisting of rimiducid (AP1903), AP20187, rapamycin, 5-fluorocytosine, ganciclovir, CB 1954, 6-methylpurine deoxyriboside, fludarabine, indole-3-acetic acid (IAA), tetracycline, doxycycline, tamoxifen, cumate, FKCsA, abscisic acid (ABA), riboswitch, ecdysone, tryptophan, arabinose, isopropyl β-d-1-thiogalactopyranoside (IPTG), asunaprevir, grazoprevir, dTAG-13, lenalidomide, anti-MHC-I antibodies, anti-MHC-II antibodies, anti-CCR4 antibodies, anti-CD16 antibodies, anti-CD19 antibodies, anti-CD20 antibodies, anti-CD30 antibodies, anti-EGFR antibodies, anti-GD2 antibodies, anti-HER1 antibodies, anti-HER2 antibodies, anti-MUC1 antibodies, anti-PSMA antibodies, and anti-RQR8 antibodies. In some embodiments, the anti-CCR4 antibodies include, but are not limited to mogamulizumab and biosimilars thereof. In some embodiments, the anti-CD16 or anti-CD30 antibodies include, but are not limited to AFM13 and biosimilars thereof. In some embodiments, the anti-CD19 antibodies include, but are not limited to, tafasitamab, loncastuximab tesirine, MOR208, and biosimilars thereof. In some embodiments, In some embodiments, anti-CD20 antibodies include, but are not limited to, rituximab, obinutuzumab, ofatumumab, ublituximab, ocaratuzumab, rituximab-R11b, and biosimilars thereof. In some embodiments, the anti-EGFR (or anti-HER1) antibodies include, but are not limited to, tomuzotuximab, R05083945 (GA201), panitumumab, cetuximab, and biosimilars thereof. In some embodiments, the anti-GD2 antibodies include, but are not limited to, dinituximab and biosimilars thereof. In some embodiments, anti-HER2 antibodies include, but are not limited to, trastuzumab, pertuzumab, and biosimilars thereof. In some embodiments, the anti-GD2 antibodies include, but are not limited to, Hu14.18K322A, Hu14.18-IL2, Hu3F8, dinituximab, c.60C3-R11c, and biosimilars thereof.

In some embodiments, the methods described herein comprise use of a selection agent, wherein the selection agent is a kill switch. In some embodiments, the kill switch is selected from the group consisting of an inducible caspase 9 (iCasp9), cytosine deaminase (CDA), herpes simplex virus thymidine kinase (HSV-Tk), rapamycin-activated caspase 9 (rapaCasp9), chemically regulated-SH2-delivered inhibitory tail (CRASH-IT), nitroreductase (NTR), purine nucleoside phosphorylase (PNP), horseradish peroxidase, inducible MHC-I, inducible MHC-II, CCR4, CD16, CD19, CD20, CD30, EGFR, GD2, HER1, HER2, MUC1, PSMA, and RQR8. In some embodiments, the kill switch is iCasp9 or CDA. In some embodiments, the inducible MHC-I is selected from the group consisting of HLA-A, HLA-B, and HLA-C. In some embodiments, the inducible MHC-II is selected from the group consisting of HLA-DP, HLA-DQ, and HLA-DR. In some embodiments, the selection agent is a degron.

In some embodiments, the methods described herein comprise use of a detection agent, wherein the detection agent is a cell surface protein. In some embodiments, the cell surface protein is selected from the group consisting of EGFR fused to a His-tag, RQRB fused to a His-tag, and CD47 fused to a His-tag. In some embodiments, the detection agent is a fluorescent protein. In some embodiments, the fluorescent protein is selected from the group consisting of: green fluorescent protein (GFP), enhanced green fluorescent protein (EGFP), yellow fluorescent protein (YFP), enhanced yellow fluorescent protein (EYFP), blue fluorescent protein (BFP), enhanced blue fluorescent protein (EBFP), cyan fluorescent protein (CFP), enhanced cyan fluorescent protein (ECFP), superfolder GFP, superfolder YFP, orange fluorescent protein, red fluorescent protein, small ultrared fluorescent protein, FMN-binding fluorescent protein, dsRed, qFP611, Dronpa, TagRFP, KFP, EosFP, IrisFP, Dendra, Kaede, KikGrl, emerald fluorescent protein, Azami Green, mWasabi, TagGFP, TurboGFP, AcGFP, ZsGreen, and T-Sapphire. In some embodiments, the detection agent is associated with a barcode. In some embodiments, the detection agent is a blood-detectable biomarker. In some embodiments, the detection agent is a secreted protein. In some embodiments, the secreted protein is detectable in vitro. In some embodiments, in vitro detection comprises detecting the secreted protein in cell culture medium collected from a cell culture comprising the cell described herein. In some embodiments, the secreted protein is detectable ex vivo. In some embodiments, ex vivo detection comprises detecting the secreted protein in a blood sample taken from an individual administered the cells described herein.

In some embodiments, the methods described herein comprises using a modified cell expressing an engineered receptor. In some embodiments, the engineered receptor is a chimeric antigen receptor (CAR). In some embodiments, the CAR comprises an extracellular antigen binding domain specifically recognizing a target antigen; a transmembrane domain; and an intracellular signaling domain. In some embodiments, the cell is an immune cell. In some embodiments, the immune cell is a T cell, a B cell, or a natural killer (NK) cell.

In some embodiments, the methods described herein comprise use of a cell, wherein the cell is a mammalian cell. In some embodiments, the mammalian cell is a human cell. In some embodiments, the cell is a stem cell-derived cell. In some embodiments, the stem cell-derived cell is derived from a cell selected from the group consisting of embryonic stem cell, induced pluripotent stem cell, multipotent stem cell, adult stem cell, hematopoietic stem cell, mesenchymal stem cell, endothelial stem cell, epithelial stem cell, adipose stem or progenitor cells, germline stem cells, lung stem or progenitor cells, mammary stem cells, olfactory adult stem cells, hair follicle stem cells, multipotent stem cells, amniotic stem cells, cord blood stem cells, neural stem, and progenitor cells. In some embodiments, the stem-cell derived cell is selected from the group consisting of a stem cell-derived beta cell, an alpha cell, and a delta cell. In some embodiments, the stem-cell derived cell is a stem cell-derived beta cell (SC-beta cell). In some embodiments, the stem cell-derived cell is a stem-cell derived beta islet cell. In some embodiments, the stem-cell derived cell is a stem cell-derived T cell. In some embodiments, the stem cell-derived T cell is selected from the group consisting of an ab T cell, dg T cell, helper/regulatory T cell, cytotoxic T cell, progenitor T cell (e.g., a progenitor T cell that is CD34+CD7+Cd1a− or CD34+CD7+CD5+Cd1a−), naive T cell, central memory T cell, effector T cell, terminal effector T cell, immature T cell, mature T cell, natural killer T cell, naive T cell, naive central memory T cell (TCM cell), effector memory T cell (TEM cell), and effector memory RA T cell (TEMRA cell). In some embodiments, the stem-cell derived cell is a stem cell-derived neural cell. In some embodiments, the stem cell-derived neural cell is selected from the group consisting of a glial cell, cerebral endothelial cell, neuron, ependymal cell, astrocyte, microglial cell, oligodendrocyte, and a Schwann cell. In some embodiments, the stem-cell derived cell is a stem cell-derived cardiac cell. In some embodiments, the stem cell-derived cardiac cell is selected from the group consisting of cardiomyocytes, nodal cardiomyocytes, conducting cardiomyocytes, working cardiomyocytes, cardiomyocyte precursors, cardiomyocyte progenitor cell, cardiac stem cell, and cardiac muscle cells.

In some embodiments, the methods described herein comprise use of modified cells comprising modifications that (i) increase expression of one or more tolerogenic factors, and (ii) reduce expression of one or more major histocompatibility complex (MHC) class I molecules and/or one or more MHC class II molecules, wherein the increased expression of (i) and the reduced expression of (ii) is relative to a cell of the same cell type that does not comprise the modifications. In some embodiments, the one or more of the modifications in (ii) reduce expression of: a. one or more MHC class I molecules; b. one or more MHC class II molecules; or c. one or more MHC class I molecules and one or more MHC class II molecules. In some embodiments, the one or more modifications reduce expression of one or more molecules selected from the group consisting of B2M, TAP I, NLRC5, CIITA, HLA-A, HLA-B, HLA-C, HLA-DP, HLA-DM, HLA-DOA, HLA-DOB, HLA-DQ, HLA-DR, RFX5, RFXANK, RFXAP, NFY-A, NFY-B and/or NFY-C and any combination thereof. In some embodiments, the engineered cell does not express one or more molecules selected from the group consisting of B2M, TAP I, NLRC5, CIITA, HLA-A, HLA-B, HLA-C, HLA-DP, HLA-DM, HLA-DOA, HLA-DOB, HLA-DQ, HLA-DR, and combinations thereof. In some embodiments, the one or more tolerogenic factors is selected from the group consisting of CD47, A20/TNFAIP3, C1-Inhibitor, CCL21, CCL22, CD16, CD16 Fc receptor, CD24, CD27, CD35, CD39, CD46, CD52, CD55, CD59, CD200, CR1, CTLA4-Ig, DUX4, FasL, H2-M3, HLA-C, HLA-E, HLA-E heavy chain, HLA-G, PD-L1, IDO1, IL-10, IL15-RF, IL-35, MANF, Mfge8, PD-1, PD-L1 or Serpinb9, and any combination thereof. In some embodiments, the one or more tolerogenic factors is selected from the group consisting of: a) CD47; b) HLA-E; c) CD24; d) PD-L1; e) CD46; f) CD55; f) CD59; h) CR1; i) MANF; j) A20/TNFAIP3; k) HLA-E and CD47; 1) CD24, CD47, PD-L1, and any combination thereof; m) HLA-E, CD24, CD47, and PD-L1, and any combination thereof; n) CD46, CD55, CD59, and CR1, and any combination thereof; o) HLA-E, CD46, CD55, CD59, and CR1, and any combination thereof; p) HLA-E, CD24, CD47, PDL1, CD46, CD55, CD59, and CR1, and any combination thereof; q) HLA-E and PDL1; r) HLA-E, PDL1, and A20/TNFAIP, and any combination thereof; sHLA-E, PDL1, and MANF, and any combination thereof; t) HLA-E, PDL1, A20/TNFAIP, and MANF, and any combination thereof; and u) CD47, PD-L1, HLA-E, HLA-G, CCL21, FASL, SERPINB9, CD200, MFGE8, and any combination thereof. In some embodiments, the engineered cell comprises modifications according to the following: (i) (a) reduces expression of MHC I and/or MHC II; (b) reduces expression of MIC-A and/or MIC-B; (c) increases expression of CD47, and optionally CD24 and PD-L1; and (d) increases expression of CD46, CD55, CD59 and CR1; (ii) (a) reduces expression of MHC class I molecule; (b) reduces expression of MIC-A and/or MIC-B; (c) reduces expression of TXNIP; (d) increases expression of PD-L1 and HLA-E; and (e) optionally increases expression of A20/TNFAIP3 and MANF; (iii) (a) increases expression of CCL21, PD-L1, FASL, SERPINB9, HLA-G, CD47, CD200, and MFGE8; and (b) reduces expression of a MICA and/or MICB; (iv) (a) reduces expression of MHC I and/or MHC II; and (b) increases expression of CD47; or (v) any of (i)-(iv) above further comprising modifications for increasing or decreasing expression of one or more additional genes, optionally reducing expression of B2M, TAP I, NLRC5, CIITA, HLA-A, HLA-B, HLA-C, HLA-DP, HLA-DM, HLA-DOA, HLA-DOB, HLA-DQ, HLA-DR, RFX5, RFXANK, RFXAP, NFY-A, NFY-B, NFY-C, CTLA-4, PD-1, IRF1, MIC-A, MIC-B, a protein that is involved in oxidative or ER stress, TRAC, TRB, CD142, ABO, CD38, PCDH11Y, NLGN4Y and/or RHD, further optionally wherein proteins that are is involved in oxidative or ER stress include thioredoxin-interacting protein (TXNIP), PKR-like ER kinase (PERK), inositol-requiring enzyme 1α (IRE1α), and DJ-1 (PARK7).

In some embodiments, the methods described herein comprise use of modified cells comprising modifications that (i) increase expression of one or more tolerogenic factors selected from the group consisting of CD47, PD-L1, HLA-E, HLA-G, CCL21, FASL, SERPINB9, CD200, MFGE8, and any combination thereof, and (ii) reduce expression of one or more major histocompatibility complex (MHC) class I molecules and/or one or more MHC class II molecules, wherein the increased expression of (i) and the reduced expression of (ii) is relative to a cell of the same cell type that does not comprise the modifications. In some embodiments, the modification(s) that increase expression comprise increased surface expression, and/or the modifications that reduce expression comprise reduced surface expression, optionally wherein there is no detectable surface expression. In some embodiments, the modification that increases expression of the one or more tolerogenic factors comprises an exogenous polynucleotide encoding the one or more tolerogenic factors. In some embodiments, the one or more tolerogenic factors comprises CD47. In some embodiments, the one or more tolerogenic factors is CD47 and the exogenous polynucleotide encoding CD47 encodes a sequence of amino acids having at least 85% identity to the amino acid sequence of SEQ ID NO: 2, and reduces innate immune killing of the engineered primary cell. In some embodiments, the exogenous polynucleotide encoding CD47 encodes a sequence set forth in SEQ ID NO: 2. In some embodiments, the exogenous polynucleotide encoding the one or more tolerogenic factors is operably linked to a promoter. In some embodiments, the promoter is a constitutive promoter. In some embodiments, the promoter is selected from the group consisting of the CAG promoter, the cytomegalovirus (CMV) promoter, the EF1a promoter, the PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein barr virus (EBV) promoter, the Rous sarcoma virus (RSV) promoter and the UBC promoter. In some embodiments, the exogenous polynucleotide encoding CD47 is integrated into the genome of the engineered primary cell. In some embodiments, the exogenous polynucleotide is a multicistronic vector encoding the one or more tolerogenic factors and an additional transgene encoding a second transgene. In some embodiments, the integration is by non-targeted insertion into the genome of the engineered primary cell, optionally by introduction of the exogenous polynucleotide into the cell using a lentiviral vector. In some embodiments, the integration is by targeted insertion into a target genomic locus of the cell. In some embodiments, the target genomic locus is a a B2M gene locus, a CIITA gene locus, a MICA locus, a MICB locus, a TRAC gene locus, or a TRBC gene locus. In some embodiments, the target genomic locus is selected from the group consisting of: a CCR5 gene locus, a CXCR4 gene locus, a PPPIR12C (also known as AAVS1) gene, an albumin gene locus, a SHS231 locus, a CLYBL gene locus, a ROSA26 gene locus, ABO gene locus, F3 gene locus, FUT1 gene locus, HMGB1 gene locus, KDM5D gene locus, LRP1 gene locus, RHD gene locus, ROSA26 gene locus, and SHS231 gene locus. In some embodiments, the modification that reduces expression of one or more MHC class I molecules reduces one or more MHC class I molecules protein expression. In some embodiments, the modification that reduces expression of one or more MHC class I molecules is a modification that reduces expression of B-2 microglobulin (B2M). In some embodiments, the modification that reduces expression of one or more MHC class I molecules comprises reduced mRNA expression of B2M. In some embodiments, the modification that reduces expression of one or more MHC class I molecules comprises reduced protein expression of B2M. In some embodiments, the modification eliminates B2M gene activity. In some embodiments, the modification comprises inactivation or disruption of both alleles of the B2M gene. In some embodiments, the modification comprises inactivation or disruption of all B2M coding sequences in the cell. In some embodiments, the inactivation or disruption comprises an indel in the B2M gene. In some embodiments, the modification is a frameshift mutation or a deletion of a contiguous stretch of genomic DNA of the B2M gene. In some embodiments, the B2M gene is knocked out. In some embodiments, the modification that reduces expression of one or more MHC class I molecules is a modification that reduces expression of an HLA-A protein, an HLA-B protein, or HLA-C protein, optionally wherein a gene encoding said HLA-A protein, an HLA-B protein, or HLA-C protein is knocked out. In some embodiments, the modification that reduces expression of one or more MHC class II molecules reduces one or more MHC class II molecules protein expression. In some embodiments, the modification that reduces expression of one or more MHC class II molecules is a modification that reduces expression of CIITA. In some embodiments, the modification that reduces expression of one or more MHC class II molecules comprises reduced mRNA expression of CIITA. In some embodiments, the modification that reduces expression of one or more MHC class II molecules comprises reduced protein expression of CIITA. In some embodiments, the modification eliminates CIITA gene activity. In some embodiments, the modification comprises inactivation or disruption of both alleles of the CIITA gene. In some embodiments, the modification comprises inactivation or disruption of all CIITA coding sequences in the cell. In some embodiments, the inactivation or disruption comprises an indel in the CIITA gene. In some embodiments, the modification that reduces expression of one or more MHC class II molecules is a modification that reduces expression of an HLA-DP protein, an HLA-DR protein, or HLA-DQ protein, optionally wherein a gene encoding said HLA-DP protein, an HLA-DR protein, or HLA-DQ protein is knocked out. In some embodiments, the modification that reduces expression of the one or more MHC class I molecules and/or the one or more MHC class II molecules is by a genome-modifying protein. In some embodiments, the genome-modifying protein is associated with gene editing by a sequence-specific nuclease, a CRISPR-associated transposase (CAST), prime editing, or Programmable Addition via Site-specific Targeting Elements (PASTE). In some embodiments, the modification by the genome-modifying protein is nuclease-mediated gene editing. In some embodiments, the nuclease-mediated gene editing is by a zinc finger nuclease (ZFN), a TAL-effector nuclease (TALEN), or a CRISPR-Cas combination that targets the B2M gene, optionally wherein the Cas is Cas9. In some embodiments, the nuclease-mediated gene editing is by a CRISPR-Cas combination and the CRISPR-Cas combination comprises a guide RNA (gRNA) having a targeting domain that is complementary to at least one target site of an endogenous gene for reducing the expression of the one or more MHC class I molecuels and/or one or more MHC class II molecules. In some embodiments, the CRISPR-Cas combination is a ribonucleoprotein (RNP) complex comprising the gRNA and a Cas protein. In some embodiments, the engineered primary cell is a human cell or an animal cell. In some embodiments, the engineered primary cell is a human cell. In some embodiments, the primary cell is a cell type that is exposed to the blood. In some embodiments, the engineered primary cell is a primary cell isolated from a donor subject. In some embodiments, the donor subject is healthy or is not suspected of having a disease or condition at the time the donor sample is obtained from the donor subject. In some embodiments, the engineered primary cell is selected from an islet cell, a beta islet cell, a pancreatic islet cell, an immune cell, a B cell, a T cell, a natural killer (NK) cell, a natural killer T (NKT) cell, a macrophage cell, an endothelial cell, a muscle cell, a cardiac muscle cell, a smooth muscle cell, a skeletal muscle cell, a dopaminergic neuron, a retinal pigmented epithelium cell, a hepatocyte, a thyroid cell, a skin cell, a glial progenitor cell, a neural cell, a cardiac cell, and a blood cell. In some embodiments, the engineered primary cell is an endothelial cell. In some embodiments, the engineered primary cell is an epithelial cell. In some embodiments, the engineered primary cell is a retinal pigmented epithelial cell. In some embodiments, the engineered primary cell is a T cell. In some embodiments, the engineered primary cell is an NK cell. In some embodiments, the engineered primary cell comprises a chimeric antigen receptor (CAR). In some embodiments, the engineered primary cell is an islet cell, optionally a beta islet cell. In some embodiments, the engineered primary cell is a hepatocyte. In some embodiments, the engineered primary cell is ABO blood group type O. In some embodiments, the engineered primary cell is Rhesus factor negative (Rh−).

In some embodiments, the methods described herein comprise use of modified cells capable of controlled killing of the engineered cell. In some embodiments, the engineered cell comprises a suicide gene or a suicide switch. In some embodiments, the suicide gene or the suicide switch induces controlled cell death in the presence of a drug or prodrug, or upon activation by a selective exogenous compound. In some embodiments, administration of an agent allows for depletion of an engineered cell of the population of engineered cells. In some embodiments, the agent recognizes the one or more tolerogenic factors on the surface of the engineered cell. In some embodiments, the engineered cell is engineered to express the one or more tolerogenic factors. In some embodiments, the one or more tolerogenic factors is CD47. In some embodiments, expression of a detection agent acts as a signal for administration of an exogenous kill switch directed against or specific to a tolerogenic agent. In some embodiments, the exogenous kill switch is an anti-CD47 antibody.

### VII. Therapeutic Uses and Products

In some embodiments, there is provided a method of treating a disease in an individual, comprising administering a cell therapy to treat the disease, wherein the cell therapy comprises a nucleic acid encoding a selection agent or a detection agent integrated at an endogenous proliferation gene locus and operably linked to the promoter of the endogenous proliferation gene.

In some embodiments, there is provided a method of treating a disease in an individual, comprising administering a cell therapy to treat the disease, wherein the cell therapy comprises a nucleic acid encoding a selection agent or a detection agent integrated at an off-target cell marker gene locus and operably linked to the promoter of the off-target cell marker gene.

In some embodiments, there is provided a method of treating a patient in need thereof comprising, (a) administering a population of cells to the patient, wherein the population of cells comprise a nucleic acid encoding a selection agent integrated at an endogenous proliferation gene locus and operably linked to the promoter of the endogenous proliferation gene, and (b) activating the selection agent to eliminate one or more cells in the population of cells. In embodiments, the step of activating the selection agent takes place before the population of cells are administered to the patient and/or after the population of cells are administered to the patient. In some embodiments, there is provided a method of treating a patient in need thereof comprising, (a) administering a population of cells to the patient, wherein the population of cells comprise a nucleic acid encoding a selection agent integrated at an off-target cell marker gene locus and operably linked to the promoter of the off-target cell marker gene, and (b) activating the selection agent to eliminate one or more cells in the population of cells. In some embodiments, the step of activating the selection agent takes place before the population of cells are administered to the patient. In some embodiments, the step of activating the selection agent takes place after the population of cells are administered to the patient. In some embodiments, the step of activating the selection agent takes place before the population of cells are administered to the patient and after the population of cells are administered to the patient.

In some embodiments, there is provided a method of treating a patient in need thereof comprising activating a selection agent to eliminate one or more cells in a population of cells, wherein the patient was previously administered the population of cells, wherein the population of cells comprise a nucleic acid encoding a selection agent integrated at an endogenous proliferation gene locus and operably linked to the promoter of the endogenous proliferation gene.

In some embodiments, there is provided a method of treating a patient in need thereof comprising activating a selection agent to eliminate one or more cells in a population of cells, wherein the patient was previously administered the population of cells, wherein the population of cells comprise a nucleic acid encoding a selection agent integrated at an off-target cell marker gene locus and operably linked to the promoter of the off-target cell marker gene.

### 1. Uses

Such methods and uses include, for example, administration of modified cells or population of said cells (e.g., T cells, B cells, NK cells, islet cells such as beta cells, or hypoimmunogenic cells thereof), or compositions containing the same, to a subject having a disease, condition, or disorder. It is within the level of a skilled artisan to choose the appropriate modified cell or population of cells as provided herein for a particular disease, condition, or disorder. In some embodiments, the modified cells or populations of cells or compositions thereof are administered in an effective amount to effect treatment of the disease or disorder, e.g., a therapeutically effective amount. Uses include uses of the modified cells or populations of cells or compositions thereof in such methods and treatments, and in the preparation of a medicament in order to carry out such therapeutic methods. In some embodiments, the methods thereby treat the disease or condition or disorder in the subject.

In some embodiments, the modified cell or population of cells (e.g., T cells) comprises a T cell receptor (TCR), as described in more detail herein. In some embodiments, the modified cell or population of cells comprises a kill switch, such as any of the kill switches described herein. In some embodiments, the modified cell or population of cells comprises a transcription factor, as described in more detail herein. In some embodiments, the engineered cell or population of cells comprises a tolerogenic factor, such as any of the tolerogenic factors described herein. In some embodiments, the tolerogenic factor is selected from the group consisting of: CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.

In some embodiments, the disease or disorder to be treated is selected from autoimmune or inflammatory disorders, including but not limited to, any of the disorders selected from the group consisting of: arthritis, rheumatoid arthritis, acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, juvenile-onset rheumatoid arthritis, osteoarthritis, arthritis chronica progrediente, arthritis deformans, polyarthritis chronica primaria, reactive arthritis, ankylosing spondylitis, inflammatory hyperproliferative skin diseases, psoriasis, plaque psoriasis, gutatte psoriasis, pustular psoriasis, psoriasis of the nails, atopy, atopic diseases, hay fever, Job's syndrome, dermatitis, contact dermatitis, chronic contact dermatitis, exfoliative dermatitis, exfoliative psoriatic dermatitis, allergic dermatitis, allergic contact dermatitis, dermatitis herpetiformis, nummular dermatitis, seborrheic dermatitis, non-specific dermatitis, primary irritant contact dermatitis, atopic dermatitis, x-linked hyper IgM syndrome, allergic intraocular inflammatory diseases, urticaria, chronic allergic urticaria, chronic idiopathic urticaria, chronic autoimmune urticaria, myositis, polymyositis/dermatomyositis, juvenile dermatomyositis, toxic epidermal necrolysis, scleroderma, systemic scleroderma, sclerosis, systemic sclerosis, multiple sclerosis (MS), MS associated with EBV infection, spino-optical MS, primary progressive MS (PPMS), relapsing-remitting MS (RRMS), progressive relapsing MS, secondary progressive MS (SPMS), progressive systemic sclerosis, atherosclerosis, arteriosclerosis, sclerosis disseminata, ataxic sclerosis, neuromyelitis optica spectrum disorder, inflammatory bowel disease (IBD), Crohn's disease, autoimmune-mediated gastrointestinal diseases, colitis, ulcerative colitis, colitis ulcerosa, microscopic colitis, collagenous colitis, colitis polyposa, necrotizing enterocolitis, transmural colitis, autoimmune inflammatory bowel disease, bowel inflammation, pyoderma gangrenosum, erythema nodosum, primary sclerosing cholangitis, respiratory distress syndrome, adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage, meningitis, herpes gestationis, pemphigoid gestationis, pruritis scroti, autoimmune premature ovarian failure, sudden hearing loss due to an autoimmune condition, IgE-mediated diseases, anaphylaxis, allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN) with or without nephrotic syndrome, chronic or acute glomerulonephritis, primary GN, immune-mediated GN, membranous GN (membranous nephropathy), idiopathic membranous GN or idiopathic membranous nephropathy, membrano- or membranous proliferative GN (MPGN), Type I or Type II GN, rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, erythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema, allergic or atopic eczema, asteatotic eczema, dyshidrotic eczema, vesicular palmoplantar eczema, asthma, asthma bronchiale, bronchial asthma, auto-immune asthma, conditions involving infiltration of T cells or chronic inflammatory responses, immune reactions against foreign antigens, immune reactions against fetal A-B-O blood groups during pregnancy, chronic pulmonary inflammatory disease, autoimmune myocarditis, leukocyte adhesion deficiency, lupus, lupus nephritis, lupus cerebritis, pediatric lupus, non-renal lupus, extra-renal lupus, discoid lupus, discoid lupus erythematosus, alopecia lupus, systemic lupus erythematosus (SLE), cutaneous SLE, subacute cutaneous SLE, neonatal lupus syndrome (NLE), lupus erythematosus disseminatus, Type I diabetes, Type II diabetes, latent autoimmune diabetes in adults, Type 1.5 diabetes, juvenile onset (Type I) diabetes mellitus, pediatric insulin-dependent diabetes mellitus (IDDM), adult onset diabetes mellitus (Type II diabetes), idiopathic diabetes, insipidus, diabetic retinopathy, diabetic nephropathy, diabetic large-artery disorder, immune responses associated with acute or delayed hypersensitivity mediated by cytokines or T-lymphocytes, tuberculosis, sarcoidosis, granulomatosis, lymphomatoid granulomatosis, Wegener's granulomatosis, agranulocytosis, vasculitides, vasculitis, large-vessel vasculitis, polymyalgia rheumatica, giant cell (Takayasu's) arteritis, medium-vessel vasculitis, Kawasaki's disease, polyarteritis nodosa/periarteritis nodosa, microscopic polyarteritis, immunovasculitis, CNS vasculitis, cutaneous vasculitis, hypersensitivity vasculitis, necrotizing vasculitis, systemic necrotizing vasculitis, ANCA-associated vasculitis, Churg-Strauss vasculitis, syndrome (CSS), ANCA-associated small-vessel vasculiti, temporal arteritis, aplastic anemia, autoimmune aplastic anemia, Coombs positive anemia, Diamond Blackfan anemia, hemolytic anemia, immune hemolytic anemia, autoimmune hemolytic anemia (AIHA), pernicious anemia (anemia perniciosa), Addison's disease, pure red cell anemia, aplasia (PRCA), Factor VIII deficiency, hemophilia A, autoimmune neutropenia, pancytopenia, leukopenia, diseases involving leukocyte diapedesis, CNS inflammatory disorders, Alzheimer's disease, Parkinson's disease, multiple organ injury syndrome, multiple organ injury syndrome secondary to septicemia, trauma, or hemorrhage, antigen-antibody complex-mediated diseases, anti-glomerular basement membrane disease, anti-phospholipid antibody syndrome, anti-phospholipid syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid, pemphigoid bullous, skin pemphigoid, pemphigus, pemphigus vulgaris, pemphigus foliaceus, pemphigus mucus-membrane pemphigoid, pemphigus erythematosus, autoimmune polyendocrinopathies, Reiter's disease or syndrome, thermal injury, preeclampsia, an immune complex disorder, immune complex nephritis, antibody-mediated nephritis, polyneuropathies, chronic neuropathy, IgM polyneuropathies, IgM-mediated neuropathy, thrombocytopenia, thrombotic thrombocytopenic purpura (TTP), post-transfusion purpura (PTP), heparin-induced thrombocytopenia, autoimmune or immune-mediated thrombocytopenia, idiopathic thrombocytopenic purpura (ITP), chronic or acute ITP, acquired thrombocytopenic purpura, scleritis, idiopathic cerato-scleritis, episcleritis, autoimmune disease of the testis or ovary, autoimmune orchitis or oophoritis, primary hypothyroidism, hypoparathyroidism, autoimmune endocrine diseases, thyroiditis, autoimmune thyroiditis, Hashimoto's disease, chronic thyroiditis, Hashimoto's thyroiditis, subacute thyroiditis, autoimmune thyroid disease, idiopathic hypothyroidism, Grave's disease, polyglandular syndromes, autoimmune polyglandular syndromes, polyglandular endocrinopathy syndromes, paraneoplastic syndromes, neurologic paraneoplastic syndromes, Lambert-Eaton myasthenic syndrome, Eaton-Lambert syndrome, stiff-man or stiff-person syndrome, encephalomyelitis, allergic encephalomyelitis, encephalomyelitis allergica, experimental allergic encephalomyelitis (EAE), myasthenia gravis, thymoma-associated myasthenia gravis, cerebellar degeneration, neuromyotonia, opsoclonus, opsoclonus myoclonus syndrome (OMS), sensory neuropathy, multifocal motor neuropathy, Sheehan's syndrome, hepatitis, autoimmune hepatitis, chronic hepatitis, lupoid hepatitis, giant cell hepatitis, chronic active hepatitis, autoimmune chronic active hepatitis, lymphoid interstitial pneumonitis (LIP), bronchiolitis obliterans, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis, primary biliary cirrhosis, pneumonocirrhosis, autoimmune enteropathy syndrome, Celiac or Coeliac disease, celiac sprue (gluten enteropathy), refractory sprue, idiopathic sprue, cryoglobulinemia, amyotrophic lateral sclerosis (ALS; Lou Gehrig's disease), coronary artery disease, autoimmune ear disease, autoimmune inner ear disease (AIED), autoimmune hearing loss, polychondritis, refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, monoclonal B cell lymphocytosis, benign monoclonal gammopathy or monoclonal gammopathy of undetermined significance, peripheral neuropathy, paraneoplastic syndrome, channelopathies, epilepsy, migraine, arrhythmia, muscular disorders, deafness, blindness, periodic paralysis, channelopathies of the CNS, autism, inflammatory myopathy, focal or segmental or focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases, autoimmune demyelinating diseases, chronic inflammatory demyelinating polyneuropathy, Dressler's syndrome, alopecia areata, alopecia totalis, CREST syndrome, calcinosis, Raynaud's phenomenon, esophageal dysmotility, sclerodactyly, telangiectasia, male or female autoimmune infertility, anti-spermatozoan antibodies, mixed connective tissue disease, Chagas' disease, rheumatic fever, recurrent abortion, farmer's lung, erythema multiforme, post-cardiotomy syndrome, post myocardial infarction cardiotomy syndrome, Cushing's syndrome, bird-fancier's lung, allergic granulomatous angiitis, benign lymphocytic angiitis, Alport's syndrome, alveolitis, allergic alveolitis, fibrosing alveolitis, interstitial lung disease, transfusion reaction, leprosy, malaria, parasitic diseases, leishmaniasis, kypanosomiasis, schistosomiasis, ascariasis, aspergillosis, Samter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis, chronic cyclitis, heterochronic cyclitis, iridocyclitis (acute or chronic), Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, non-malignant thymoma, vitiligo, toxic-shock syndrome, food poisoning, conditions involving infiltration of T cells, leukocyte-adhesion deficiency, immune responses associated with acute or delayed hypersensitivity mediated by cytokines or T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, sphenoid sinusitis, an eosinophil-related disorder, eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis, central nervous system inflammatory disorders, ocular or orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, emphysema, alopecia areata, adipose tissue inflammation/diabetes type II, obesity-associated adipose tissue inflammation/insulin resistance, endometriosis, and pulmonary hemosiderosis.

In some embodiments, the disease or disorder is selected from the group consisting of: diabetes, cancer, vascularization disorders, ocular disease, thyroid disease, skin diseases, liver diseases, a condition or disease associated with a vascular condition or disease, a vascular condition or disease, a condition or disease associated with autoimmune thyroiditis, autoimmune thyroiditis, a condition or disease associated with a liver disease, liver disease, cirrhosis of the liver, a condition or disease associated with a corneal disease, corneal disease, Fuchs dystrophy or congenital hereditary endothelial dystrophy, a condition or disease associated with a kidney disease, kidney disease, a disease associated with cancer, cancer, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, bladder cancer, a condition or disease associated with a hematopoietic disease or disorder, a hematopoietic disease or disorder, myelodysplasia, aplastic anemia, Fanconi anemia, paroxysmal nocturnal hemoglobinuria, Sickle cell disease, Diamond Blackfan anemia, Schachman Diamond disorder, Kostmann's syndrome, chronic granulomatous disease, adrenoleukodystrophy, leukocyte adhesion deficiency, hemophilia, thalassemia, beta-thalassemia, leukaemia, acute lymphocytic leukemia (ALL), acute myelogenous (myeloid) leukemia (AML), adult lymphoblastic leukaemia, chronic lymphocytic leukemia (CLL), B-cell chronic lymphocytic leukemia (B-CLL), chronic myeloid leukemia (CML), juvenile chronic myelogenous leukemia (CML), juvenile myelomonocytic leukemia (JMML), severe combined immunodeficiency disease (SCID), X-linked severe combined immunodeficiency, Wiskott-Aldrich syndrome (WAS), adenosine-deaminase (ADA) deficiency, chronic granulomatous disease, Chediak-Higashi syndrome, Hodgkin's lymphoma, non-Hodgkin's lymphoma (NHL), AIDS, a condition or disease associated with leukemia or myeloma, leukemia, myeloma, a condition or disease associated with an autoimmune disease or condition, an autoimmune disease or condition, acute disseminated encephalomyelitis, acute hemorrhagic leukoencephalitis, Addison's disease, Agammaglobulinemia, alopecia areata, amyotrophic lateral sclerosis, ankylosing spondylitis, antiphospholipid syndrome, antisynthetase syndrome, atopic allergy, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, Balo disease, Balo concentric sclerosis, Bechets syndrome, Berger's disease, Bickerstaff's encephalitis, Blau syndrome, bullous pemphigoid, cancer, Castleman's disease, celiac disease, chronic inflammatory demyelinating polyneuropathy, chronic recurrent multifocal osteomyelitis, Churg-Strauss syndrome, cicatricial pemphigoid, Cogan syndrome, cold agglutinin disease, complement component 2 deficiency, cranial arteritis, CREST syndrome, Crohn's disease, Cushing's syndrome, cutaneous leukocytoclastic angiitis, Dego's disease, Dercum's disease, dermatitis herpetiformis, dermatomyositis, diabetes mellitus type 1, diffuse cutaneous systemic sclerosis, Dressler's syndrome, discoid lupus erythematosus, eczema, enthesitis-related arthritis, eosinophilic fasciitis, eosinophilic gastroenteritis, epidermolysis bullosa acquisita, erythema nodosum, essential mixed cryoglobulinemia, Evan's syndrome, fibrodysplasia ossificans progressiva, fibrosing alveolitis, gastritis, gastrointestinal pemphigoid, giant cell arteritis, glomerulonephritis, goodpasture's syndrome, Grave's disease, Guillain-Barre syndrome (GBS), Hashimoto's encephalitis, Hashimoto's thyroiditis, hemolytic anaemia, Henoch-Schonlein purpura, herpes gestationis, hypogammaglobulinemia, idiopathic inflammatory demyelinating disease, idiopathic pulmonary fibrosis, idiopathic thrombocytopenic purpura, IgA nephropathy, inclusion body myositis, inflammatory demyelinating polyneuropathy, interstitial cystitis, juvenile idiopathic arthritis, juvenile rheumatoid arthritis, Kawasaki's disease, Lambert-Eaton myasthenic syndrome, leukocytoclastic vasculitis, lichen planus, lichen sclerosus, linear IgA disease (LAD), Lou Gehrig's disease, lupoid hepatitis, lupus erythematosus, Majeed syndrome, Meniere's disease, microscopic polyangiitis, Miller-Fisher syndrome, mixed connective tissue disease, morphea, Mucha-Habermann disease, multiple sclerosis, myasthenia gravis, myositis, neuropyelitis optica, neuromyotonia, ocular cicatricial pemphigoid, opsoclonus myoclonus syndrome, ord thyroiditis, palindromic rheumatism, paraneoplastic cerebellar degeneration, paroxysmal nocturnal hemoglobinuria (PNH), Parry Romberg syndrome, Parsonnage-Turner syndrome, pars planitis, pemphigus, pemphigus vulgaris, pernicious anemia, perivenous encephalomyelitis, POEMS syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatoid fever, sarcoidosis, Schmidt syndrome, Schnitzler syndrome, scleritis, scleroderma, Sjogren's syndrome, spondylarthropathy, Still's disease, stiff person syndrome, subacute bacterial endocarditis, Susac's syndrome, Sweet's syndrome, Sydenham chorea, sympathetic ophthalmia, Takayasu's arteritis, temporal arteritis, Tolosa-Hunt syndrome, transverse myelitis, ulcerative colitis, undifferentiated connective tissue disease, undifferentiated spondylarthropathy, vasculitis, vitiligo, Wegener's granulomatosis, a condition or disease associated with Parkinson's disease, Huntington disease, Alzheimer's disease, multiple sclerosis, Amyotrophic lateral sclerosis (ALS), spinocerebellar ataxia, dementia, Charcot-Marie-Tooth disease, prion disease, muscular dystrophy, other neurodegenerative disease or condition, attention deficit hyperactivity disorder (ADHD), Tourette Syndrome (TS), schizophrenia, psychosis, depression, bipolar disorder, anxiety disorder, autism spectrum disorder, other neuropsychiatric disorder, and stroke.

In some embodiments, the modified cells (e.g., T cells, B cells, NK cells, islet cells such as beta cells, or hypoimmunogenic cells thereof) provided herein, populations of said modified cells, or compositions thereof can be used in cell therapy. Therapeutic cells outlined herein may be useful to treat a disorder such as, but not limited to, a cancer, a genetic disorder, a chronic infectious disease, an autoimmune disorder, a neurological disorder, or the like.

In some embodiments, the modified cells (e.g., T cells, B cells, NK cells, islet cells such as beta cells, or hypoimmunogenic cells thereof) provided herein, populations of said modified cells, or compositions thereof are administered prior to providing a tissue, organ, or partial organ transplant to a patient in need thereof. In some embodiments, the patient exhibits a reduced immune response to the modified cells or populations of cells. In particular embodiments, the patient does not exhibit an immune response to the modified cells or populations of cells. In certain embodiments, the modified cells or populations of cells are administered to the patient for the treatment of a cellular deficiency in a particular tissue or organ, and the patient subsequently receives a tissue or organ transplant for the same particular tissue or organ. In such embodiments, the engineered cell treatment functions as a bridge therapy to the eventual tissue or organ replacement. For example, in some embodiments, the patient has a liver disorder and receives an engineered hepatocyte treatment as provided herein, prior to receiving a liver transplant. In certain embodiments, the engineered cells or populations of cells are administered to the patient for the treatment of a cellular deficiency in a particular tissue or organ and the patient subsequently receives a tissue or organ transplant for a different tissue or organ. For example, in some embodiments, the patient is a diabetes patient who is treated with engineered pancreatic beta cells as provided herein prior to receiving a pancreas transplant. In some embodiments, the method is for the treatment of a cellular deficiency. In exemplary embodiments, the tissue or organ transplant is a heart transplant, a lung transplant, a kidney transplant, a liver transplant, a pancreas transplant, an intestine transplant, a stomach transplant, a cornea transplant, a bone marrow transplant, a blood vessel transplant, a heart valve transplant, or a bone transplant.

The modified cells (e.g., T cells, B cells, NK cells, islet cells such as beta cells, or hypoimmunogenic cells thereof) provided herein, populations of said engineered cells, or compositions thereof can be administered to any suitable patients including, for example, a candidate for a cellular therapy for the treatment of a disease or disorder, such as any of the diseases or disorders described herein. Candidates for cellular therapy include any patient having a disease or condition that may potentially benefit from the therapeutic effects of the engineered cells or population of cells described herein. In some embodiments, the patient is an allogeneic recipient of the administered cells. In some embodiments, the provided engineered cells or populations of cells are effective for use in allogeneic cell therapy. A candidate who benefits from the therapeutic effects of the engineered cells or populations of cells provided herein may exhibit an elimination, reduction, or amelioration of the disease or condition.

The specific amount and dosage regimen of engineered cells (e.g., T cells, B cells, NK cells, islet cells such as beta cells, or hypoimmunogenic cells thereof) provided herein, populations of said engineered cells, or compositions thereof will vary depending on the weight, gender, age, and health of the individual; the formulation; the biochemical nature; bioactivity; bioavailability and side effects of the cells; and the number and identity of the cells in the complete therapeutic regimen.

The engineered cells (e.g., T cells, B cells, NK cells, islet cells such as beta cells, or hypoimmunogenic cells thereof) provided herein, populations of said engineered cells, or compositions thereof can be administered by any suitable route, such as by intravenous, intraperitoneal, or subcutaneous administration, as described in Section IV (“Modified Cell Compositions”) above. In some embodiments, the engineered cells provided herein, populations of said engineered cells, or compositions thereof are administered parenterally. In some embodiments, the engineered cells provided herein, populations of said engineered cells, and compositions thereof are administered to a subject intravenously.

Any therapeutically effective amount of engineered cells (e.g., T cells, B cells, NK cells, islet cells such as beta cells, or hypoimmunogenic cells thereof) provided herein, and populations of said engineered cells, can be included in compositions for therapeutic treatments or uses, depending on the indication being treated. In some embodiments, the pharmaceutical composition includes at least about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells. In some embodiments, the pharmaceutical composition includes up to about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells. In some embodiments, the pharmaceutical composition includes up to about 6.0×10⁸ cells. In some embodiments, the pharmaceutical composition includes up to about 8.0×10⁸ cells. In some embodiments, the pharmaceutical composition includes at least about 1×10²-5×10²; 5×10²-1×10³; 1×10³-5×10³; 5×10³-1×10⁴; 1×10⁴-5×10⁴; 5×10⁴-1×10⁵; 1×10⁵-5×10⁵; 5×10⁵-1×10⁶; 1×10⁶-5×10⁶; 5×10⁶-1×10⁷; 1×10⁷-5×10⁷; 5×10⁷-1×10⁸; 1×10⁸-5×10⁸; 5×10⁸-1×10⁹; 1×10⁹-5×10⁹; 5×10⁹-1×10¹⁰; or 1×10¹⁰-5×10¹⁰ cells. In exemplary embodiments, the pharmaceutical composition includes from about 1.0×10⁶ to about 2.5×10⁸ cells. In some embodiments, the pharmaceutical composition comprises engineered beta cells, wherein the dose is from about 1×10⁷ cells to about 3×10⁸ cells, or from about 6,500 islet equivalents (IEQ) to about 600,000 IEQ (e.g., from about 6,500 IEQ to about 7,500 IEQ, from about 7,000 IEQ to about 8,000 IEQ, from about 7,500 IEQ to about 10,000 IEQ, from about 10,000 IEQ to about 20,000 IEQ, from about 15,000 IEQ to about 25,000 IEQ, from about 20,000 IEQ to about 50,000 IEQ, from about 25,000 IEQ to about 75,000 IEQ, from about 50,000 IEQ to about 150,000 IEQ, from about 100,000 IEQ to about 200,000 IEQ, from about 150,000 IEQ to about 250,000 IEQ, from about 200,000 IEQ to about 400,000 IEQ, from about 300,000 IEQ to about 500,000 IEQ, or from about 400,000 IEQ to about 600,000 IEQ). In some embodiments, the pharmaceutical composition has a volume of at least about any of 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 mL. In exemplary embodiments, the pharmaceutical composition has a volume of up to about any of 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 mL. In exemplary embodiments, the pharmaceutical composition has a volume of about any of 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 mL. In some embodiments, the pharmaceutical composition has a volume of from about 1-50 mL, 50-100 mL, 100-150 mL, 150-200 mL, 200-250 mL, 250-300 mL, 300-350 mL, 350-400 mL, 400-450 mL, or 450-500 mL. In some embodiments, the pharmaceutical composition has a volume of from about 1-50 mL, 50-100 mL, 100-150 mL, 150-200 mL, 200-250 mL, 250-300 mL, 300-350 mL, 350-400 mL, 400-450 mL, or 450-500 mL. In some embodiments, the pharmaceutical composition has a volume of from about 1-10 mL, 10-20 mL, 20-30 mL, 30-40 mL, 40-50 mL, 50-60 mL, 60-70 mL, 70-80 mL, 70-80 mL, 80-90 mL, or 90-100 mL. In some embodiments, the pharmaceutical composition has a volume that ranges from about 5 mL to about 80 mL. In exemplary embodiments, the pharmaceutical composition has a volume that ranges from about 10 mL to about 70 mL. In many embodiments, the pharmaceutical composition has a volume that ranges from about 10 mL to about 50 mL. In some embodiments, a dose of the pharmaceutical composition includes about 1.0×10⁵ to about 2.5×10⁸ cells at a volume of about 10 mL to 50 mL, and the pharmaceutical composition is administered as a single dose.

In some embodiments, the pharmaceutical composition is administered as a single dose of from about 1.0×10⁵ to about 1.0×10⁷ cells per kg body weight for subjects 50 kg or less. In some embodiments, the pharmaceutical composition is administered as a single dose of from about 0.5×10⁵ to about 1.0×10⁷, about 1.0×10⁵ to about 1.0×10⁷, about 1.0×10⁵ to about 1.0×10⁷, about 5.0×10⁵ to about 1×10⁷, about 1.0×10⁶ to about 1×10⁷, about 5.0×10⁶ to about 1.0×10⁷, about 1.0×10⁵ to about 5.0×10⁶, about 1.0×10⁵ to about 1.0×10⁶, about 1.0×10⁵ to about 5.0×10⁵, about 1.0×10⁵ to about 5.0×10⁶, about 2.0×10⁵ to about 5.0×10⁶, about 3.0×10⁵ to about 5.0×10⁶, about 4.0×10⁵ to about 5.0×10⁶, about 5.0×10⁵ to about 5.0×10⁶, about 6.0×10⁵ to about 5.0×10⁶, about 7.0×10⁵ to about 5.0×10⁶, about 8.0×10⁵ to about 5.0×10⁶, or about 9.0×10⁵ to about 5.0×10⁶ cells per kg body weight for subjects 50 kg or less. In some embodiments, the dose is from about 0.2×10⁶ to about 5.0×10⁶ cells per kg body weight for subjects 50 kg or less. In many embodiments, the dose is at a range that is lower than from about 0.2×10⁶ to about 5.0×10⁶ cells per kg body weight for subjects 50 kg or less. In many embodiments, the dose is at a range that is higher than from about 0.2×10⁶ to about 5.0×10⁶ cells per kg body weight for subjects 50 kg or less. In exemplary embodiments, the single dose is at a volume of about 10 mL to 50 mL. In some embodiments, the dose is administered intravenously. In exemplary embodiments, the cells are administered in a single dose of from about 1.0×10⁶ to about 5.0×10⁸ cells for subjects above 50 kg. In some embodiments, the pharmaceutical composition is administered as a single dose of from about 0.5×10⁶ to about 1.0×10¹, about 1.0×10⁶ to about 1.0×10¹, about 1.0×10⁶ to about 1.0×10¹, about 5.0×10⁶ to about 1.0×10¹, about 1.0×10⁷ to about 1.0×10¹, about 5.0×10⁷ to about 1.0×10¹, about 1.0×10⁶ to about 5.0×10⁷, about 1.0×10⁶ to about 1.0×10⁷, about 1.0×10⁶ to about 5.0×10⁷, about 1.0×10⁷ to about 5.0×10⁸, about 2.0×10⁷ to about 5.0×10⁸, about 3.0×10⁷ to about 5.0×10⁸, about 4.0×10⁷ to about 5.0×10⁸, about 5.0×10⁷ to about 5.0×10⁸, about 6.0×10⁷ to about 5.0×10⁸, about 7.0×10⁷ to about 5.0×10⁸, about 8.0×10⁷ to about 5.0×10⁸, or about 9.0×10⁷ to about 5.0×10⁸ cells per kg body weight for subjects 50 kg or less. In certain embodiments, the cells are administered in a single dose of about 1.0×10⁷ to about 2.5×10⁸ cells for subjects above 50 kg. In some embodiments, the cells are administered in a single dose of a range that is less than about 1.0×10⁷ to about 2.5×10⁸ cells for subjects above 50 kg. In some embodiments, the cells are administered in a single dose of a range that is higher than about 1.0×10⁷ to about 2.5×10⁸ cells for subjects above 50 kg. In some embodiments, the pharmaceutical composition comprises engineered beta cells, wherein the dose is from about 1.25×10⁵ cells/kg to about 1.2×10⁷ cells/kg, or from about 80 IEQ/kg to about 24,000 IEQ/kg (e.g., from about 80 IEQ/kg to about 800 IEQ/kg, from about 500 IEQ/kg to about 1500 IEQ/kg, from about 1000 IEQ/kg to about 2000 IEQ/kg, from about 1500 IEQ/kg to about 3000 IEQ/kg, from about 2000 IEQ/kg to about 4000 IEQ/kg, from about 3000 IEQ/kg to about 5000 IEQ/kg from about 4000 IEQ/kg to about 6000 IEQ/kg, from about 5000 IEQ/kg to about 10,000 IEQ/kg, from about 7500 IEQ/kg to about 15,000 IEQ/kg, from about 15,000 IEQ/kg to about 20,000 IEQ/kg, or from about 20,000 IEQ/kg to about 24,000 IEQ/kg). In some embodiments, the dose is administered intravenously. In exemplary embodiments, the single dose is at a volume of about 10 mL to 50 mL. In some embodiments, the dose is administered intravenously.

In some embodiments, the dose is administered intravenously at a rate of about 1 to 50 mL per minute, 1 to 40 mL per minute, 1 to 30 mL per minute, 1 to 20 mL per minute, 10 to 20 mL per minute, 10 to 30 mL per minute, 10 to 40 mL per minute, 10 to 50 mL per minute, 20 to 50 mL per minute, 30 to 50 mL per minute, or 40 to 50 mL per minute. In numerous embodiments, the pharmaceutical composition is stored in one or more infusion bags for intravenous administration. In some embodiments, the dose is administered completely at no more than 10 minutes, 15 minutes, 20 minutes, 25 minutes, 30 minutes, 35 minutes, 40 minutes, 45 minutes, 50 minutes, 55 minutes, 60 minutes, 70 minutes, 80 minutes, 90 minutes, 120 minutes, 150 minutes, 180 minutes, 240 minutes, or 300 minutes.

In some embodiments, a single dose of the pharmaceutical composition is present in a single infusion bag. In other embodiments, a single dose of the pharmaceutical composition is divided into 2, 3, 4, or 5 separate infusion bags.

In some embodiments, the cells described herein are administered in a plurality of doses such as 2, 3, 4, 5, 6, or more doses. In some embodiments, each dose of the plurality of doses is administered to the subject ranging from 1 to 24 hours apart. In some instances, a subsequent dose is administered from about 1 hour to about 24 hours (e.g., about any of 1, 2, 3, 4, 5, 6, 7, 8, 9, 10, 11, 12, 13, 14, 15, 16, 17, 18, 19, 20, 21, 22, 23, or 24 hours) after an initial or preceding dose. In some embodiments, each dose of the plurality of doses is administered to the subject ranging from about 1 day to about 28 days apart. In some instances, a subsequent dose is administered from about 1 day to about 28 days (e.g., about any of 1, 2, 3, 4, 5, 6, 7, 8, 9, 10, 11, 12, 13, 14, 15, 16, 17, 18, 19, 20, 21, 22, 23, 24, 25, 26, 27, or 28 days) after an initial or preceding dose. In certain embodiments, each dose of the plurality of doses is administered to the subject ranging from about 1 week to about 6 weeks apart. In certain instances, a subsequent dose is administered from about 1 week to about 6 weeks (e.g., about any of 1, 2, 3, 4, 5, or 6 weeks) after an initial or preceding dose. In several embodiments, each dose of the plurality of doses is administered to the subject ranging from about 1 month to about 12 months apart. In several instances, a subsequent dose is administered from about 1 month to about 12 months (e.g., about any of 1, 2, 3, 4, 5, 6, 7, 8, 9, 10, 11, or 12 months) after an initial or preceding dose.

In some embodiments, a subject is administered a first dosage regimen at a first timepoint, and then subsequently administered a second dosage regimen at a second timepoint. In some embodiments, the first dosage regimen is the same as the second dosage regimen. In other embodiments, the first dosage regimen is different than the second dosage regimen. In some instances, the number of cells in the first dosage regimen and the second dosage regimen are the same. In some instances, the number of cells in the first dosage regimen and the second dosage regimen are different. In some cases, the number of doses of the first dosage regimen and the second dosage regimen are the same. In some cases, the number of doses of the first dosage regimen and the second dosage regimen are different.

In some embodiments, a subject, individual, or patient to be treated according to the methods or therapeutic uses described herein is a mammal. In some embodiments, the subject, individual, or patient to be treated according to the methods or therapeutic uses described herein is a human.

In some embodiments, the engineered cells or populations of cells provided herein (e.g., engineered iPSCs, stem cells, pluripotent cells, or the like) are differentiated into different cell types for subsequent transplantation into recipient subjects. Differentiation can be assayed as is known in the art, generally by evaluating the presence of cell-specific markers. As will be appreciated by those in the art, the differentiated engineered cell derivatives can be transplanted using techniques known in the art that depends on both the cell type and the ultimate use of these cells. Exemplary types of differentiated cells and methods for producing the same are described below. In some embodiments, the engineered cells or populations of cells provided herein (e.g., engineered iPSCs, stem cells, pluripotent cells, or the like) may be differentiated to any type of cell described herein, including any described herein. In some embodiments, the differentiated engineered cells or populations of cells are transplanted or administered to a patient, e.g., as described above. In some embodiments, the patient is a human individual. In some embodiments, the patient is the same as the donor. In some embodiments, the patient is a different individual than the donor. In some embodiments, the engineered cells or populations of cells provided herein (e.g., engineered iPSCs, stem cells, pluripotent cells, or the like) are differentiated into cell types selected from T cells, NK cells, beta islet cells, endothelial cells, epithelial cells such as RPE, thyroid, skin, or hepatocytes. In some embodiments, host cells such as non-pluripotent cells (e.g., fibroblasts) from an individual donor or a pool of individual donors are isolated or obtained, generated into iPSCs in which the iPSCs are then engineered (e.g., genetic modifications) as described herein, differentiated into a desired cell type, and administered and/or transplanted into the individual patient. In some embodiments, the population of engineered cells isolated from one or more individual donors (e.g., healthy donors) or engineered cells differentiated from engineered iPSCs, stem cells, pluripotent cells, or the like derived from one or more individual donors (e.g., healthy donors), are maintained in culture, in some cases expanded, prior to administration. In certain embodiments, the population of engineered cells are cryopreserved prior to administration.

In some embodiments, the engineered cells or populations of cells described herein, such as cells isolated from one or more individual donors (e.g., healthy donors) or engineered cells differentiated from engineered iPSCs, stem cells, pluripotent cells, or the like derived from one or more individual donors (e.g., healthy donors), do not activate an immune response in the patient (e.g., in the recipient upon administration). In some embodiments, the number of cells administered is at a lower dosage than would be required for immunogenic cells (e.g., a population of cells of the same or similar cell type or phenotype but that do not contain the vectors comprising the one or more transgenes (e.g., a kill switch described herein). In some embodiments, the number of cells administration is at a lower dosage than would be required to reduce immune rejection of immunogenic cells (e.g., a population of cells of the same or similar cell type or phenotype but that do not contain the vectors comprising the one or more transgenes (e.g., a kill switch described herein).

In some embodiments, the engineered cells or populations of cells for use as described herein are pancreatic cells, such as islet cells, e.g., beta islet cells, including primary cells, which may be transplanted (either as a cell suspension or within a gel matrix as discussed herein) into the portal vein/liver, the omentum, the gastrointestinal mucosa, the bone marrow, a muscle, or subcutaneous pouches. In some embodiments, pancreatic cells described herein are administered to a subject to treat diabetes or other diseases, disorders or conditions associated with pancreatic dysfunction.

In some embodiments, the engineered cells or populations of cells for use as described herein are hepatocytes, including primary hepatocytes, which can be administered as a cell therapy to address loss of hepatocyte function, diseases of the liver, liver dysfunction, or cirrhosis of the liver.

In some embodiments, the engineered cells or populations of cells for use as described herein are immune cells (e.g., T cells, B cells, NK cells, macrophages, neutrophils, dendritic cells, MDSCs, or the like). Immune cells such as those provided herein may be useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, or bladder cancer.

In some embodiments, the engineered cells or populations of cells for use as described herein are endothelial cells, including primary endothelial cells. In some embodiments, the endothelial cells are endothelial colony forming cells (ECFCs) that may be used to form new blood vessels to address peripheral arterial disease. The engineered endothelial cells provided herein may be useful in the treatment of cardiovascular disease, vascular disease, peripheral vascular disease, ischemic disease, myocardial infarction, congestive heart failure, peripheral vascular obstructive disease, stroke, reperfusion injury, limb ischemia, neuropathy (e.g., peripheral neuropathy or diabetic neuropathy), organ failure (e.g., liver failure, kidney failure, or the like), diabetes, rheumatoid arthritis, osteoporosis, vascular injury, tissue injury, hypertension, angina pectoris and myocardial infarction due to coronary artery disease, renal vascular hypertension, renal failure due to renal artery stenosis, claudication of the lower extremities, or the like. In certain embodiments, the patient has suffered from or is suffering from a transient ischemic attack or stroke, which in some cases, may be due to cerebrovascular disease. In some embodiments, the engineered endothelial cells are administered to treat tissue ischemia, e.g., as occurs in atherosclerosis, myocardial infarction, or limb ischemia and to repair injured blood vessels. In some instances, the cells are used in bioengineering grafts. For instance, the engineered endothelial cells can be used in cell therapy for the repair of ischemic tissues, formation of blood vessels and heart valves, engineering of artificial vessels, repair of damaged vessels, or inducing the formation of blood vessels in engineered tissues (e.g., prior to transplantation). Additionally, the endothelial cells can be engineered to deliver agents to target and treat tumors. In some embodiments, the engineered endothelial cells of the disclosure may be useful for repair or replacement of tissue in need of vascular cells or vascularization. Such repair or replacement may involve administering to a human patient in need of such treatment, a composition containing the engineered endothelial cells, such as engineered isolated primary endothelial cells or differentiated endothelial cells, to promote vascularization in such tissue. The tissue in need of vascular cells or vascularization can be (e.g.) a cardiac tissue, liver tissue, pancreatic tissue, renal tissue, muscle tissue, neural tissue, or bone tissue, among others, which can be a tissue damaged and/or characterized by excess cell death, a tissue at risk for damage, or an artificially engineered tissue. In some embodiments, vascular diseases, which may be associated with cardiac diseases or disorders, can be treated by administering the engineered endothelial cells of the disclosure, such as but not limited to, definitive vascular endothelial cells, or endocardial endothelial cells derived as described herein. Such vascular diseases include, but are not limited to, coronary artery disease, cerebrovascular disease, aortic stenosis, aortic aneurysm, peripheral artery disease, atherosclerosis, varicose veins, angiopathy, infarcted area of heart lacking coronary perfusion, non-healing wounds, diabetic or non-diabetic ulcers, or any other disease or disorder in which it is desirable to induce formation of blood vessels. In certain embodiments, the engineered endothelial cells may be used for improving prosthetic implants (e.g., vessels made of synthetic materials such as Dacron and Gortex) which are used in vascular reconstructive surgery. For example, prosthetic arterial grafts are often used to replace diseased arteries which perfuse vital organs or limbs. In other embodiments, the engineered endothelial cells are used to cover the surface of prosthetic heart valves to decrease the risk of the formation of emboli by making the valve surface less thrombogenic. The engineered endothelial cells as outlined can be transplanted into the patient using well known surgical techniques for grafting tissue and/or isolated cells into a vessel. In some embodiments, the cells are introduced into the patient's heart tissue by injection (e.g., intramyocardial injection, intracoronary injection, trans-endocardial injection, trans-epicardial injection, percutaneous injection), infusion, grafting, or implantation. In certain embodiments, the engineered endothelial cells can be engineered in a way that improves their performance in the context of an implanted graft. Non-limiting illustrative examples include secretion or expression of a thrombolytic agent to prevent intraluminal clot formation, secretion of an inhibitor of smooth muscle proliferation to prevent luminal stenosis due to smooth muscle hypertrophy, and expression and/or secretion of an endothelial cell mitogen or autocrine factor to stimulate endothelial cell proliferation and improve the extent or duration of the endothelial cell lining of the graft lumen. In some embodiments, the engineered endothelial cells are utilized for delivery of therapeutic levels of a secreted product to a specific organ or limb. For example, a vascular implant lined with endothelial cells engineered (transduced) in vitro can be grafted into a specific organ or limb. The secreted product of the transduced endothelial cells will be delivered in high concentrations to the perfused tissue, thereby achieving a desired effect to a targeted anatomical location. In other embodiments, the engineered endothelial cells comprise an exogenous sequence that encodes a gene that disrupts or inhibits angiogenesis when expressed by endothelial cells in a vascularizing tumor. In some embodiments, endothelial cells described herein, such as isolated primary endothelial cells or differentiated endothelial cells, are administered to a recipient subject to treat a vascular disorder selected from vascular injury, cardiovascular disease, vascular disease, peripheral vascular disease, ischemic disease, myocardial infarction, congestive heart failure, peripheral vascular obstructive disease, hypertension, ischemic tissue injury, reperfusion injury, limb ischemia, stroke, neuropathy (e.g., peripheral neuropathy or diabetic neuropathy), organ failure (e.g., liver failure, kidney failure, or the like), diabetes, rheumatoid arthritis, osteoporosis, cerebrovascular disease, hypertension, angina pectoris and myocardial infarction due to coronary artery disease, renal vascular hypertension, renal failure due to renal artery stenosis, claudication of the lower extremities, or other vascular conditions or diseases.

In some embodiments, the engineered cells or populations of cells for use as described herein are cardiac cells, including primary cardiac cells, which may be useful for treating any of the diseases or disorders selected from the group consisting of: pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, idiopathic cardiomyopathy, other cardiomyopathy, myocardial ischemic reperfusion injury, ventricular dysfunction, heart failure, congestive heart failure, coronary artery disease, end-stage heart disease, atherosclerosis, ischemia, hypertension, restenosis, angina pectoris, rheumatic heart, arterial inflammation, cardiovascular disease, myocardial infarction, myocardial ischemia, congestive heart failure, myocardial infarction, cardiac ischemia, cardiac injury, myocardial ischemia, vascular disease, acquired heart disease, congenital heart disease, congenital heart defect, atherosclerosis, coronary artery disease, dysfunctional conduction systems, dysfunctional coronary arteries, pulmonary hypertension, cardiac arrhythmias, muscular dystrophy, muscle mass abnormality, muscle degeneration, myocarditis, infective myocarditis, drug- or toxin-induced muscle abnormalities, hypersensitivity myocarditis, autoimmune endocarditis, heart valve disease or dysfunction, endocarditis, rheumatic fever, mitral valve prolapse, infective endocarditis, cardiomegaly, and/or mitral insufficiency, among others.

In some embodiments, the engineered cells or populations of cells for use as described herein are neural cells, including primary neural cells, such as cerebral endothelial cells, dopaminergic neurons, or glial cells. The engineered neural cells of the disclosure may be useful for treating, e.g., Parkinson's disease, Alzheimer's disease, Huntington disease, multiple sclerosis, other neurodegenerative disease or condition, attention deficit hyperactivity disorder (ADHD), stroke, amyotrophic lateral sclerosis (ALS), Tourette Syndrome (TS), schizophrenia, psychosis, depression, cerebral hemorrhage, epileptic seizures, or other neuropsychiatric disorders or conditions. In some embodiments, neural cells described herein are administered to a subject to treat or ameliorate stroke. In some embodiments, engineered neurons and glial cells are administered to a subject with amyotrophic lateral sclerosis (ALS). In some embodiments, engineered cerebral endothelial cells are administered to alleviate the symptoms or effects of cerebral hemorrhage. In some embodiments, engineered dopaminergic neurons are administered to a patient with Parkinson's disease. In some embodiments, engineered noradrenergic neurons and/or GABAergic interneurons are administered to a patient who has experienced an epileptic seizure. In some embodiments, engineered motor neurons, interneurons, Schwann cells, oligodendrocytes, and/or microglia are administered to a patient who has experienced a spinal cord injury. In some embodiments, engineered dopaminergic (DA) neurons derived from HIP cells are administered to a patient, e.g., human patient, to treat a neurodegenerative disease or condition, such as Parkinson's disease, Huntington disease, or multiple sclerosis. In other embodiments, the DA neurons are used to treat or ameliorate one or more symptoms of a neuropsychiatric disorder, such as attention deficit hyperactivity disorder (ADHD), Tourette Syndrome (TS), schizophrenia, psychosis, or depression. In yet other embodiments, the DA neurons are used to treat a patient with impaired DA neurons.

In some embodiments, the engineered cells or populations of cells for use as described herein are hematopoietic stem cells, including primary hematopoietic stem cells. In some embodiments, the engineered hematopoietic stem cells may be useful for treating hematopoietic diseases or disorders, such as any selected from the group consisting of: myelodysplasia, aplastic anemia, Fanconi anemia, paroxysmal nocturnal hemoglobinuria, Sickle cell disease, Diamond Blackfan anemia, Schachman Diamond disorder, Kostmann's syndrome, chronic granulomatous disease, adrenoleukodystrophy, leukocyte adhesion deficiency, hemophilia, thalassemia, beta-thalassemia, leukaemia such as acute lymphocytic leukemia (ALL), acute myelogenous (myeloid) leukemia (AML), adult lymphoblastic leukaemia, chronic lymphocytic leukemia (CLL), B-cell chronic lymphocytic leukemia (B-CLL), chronic myeloid leukemia (CML), juvenile chronic myelogenous leukemia (CML), juvenile myelomonocytic leukemia (JMML), severe combined immunodeficiency disease (SCID), X-linked severe combined immunodeficiency, Wiskott-Aldrich syndrome (WAS), adenosine-deaminase (ADA) deficiency, chronic granulomatous disease, Chediak-Higashi syndrome, Hodgkin's lymphoma, non-Hodgkin's lymphoma (NHL), or AIDS. In some embodiments, the engineered hematopoietic stem cells may be useful for treating a cellular deficiency associated with leukemia or myeloma, or to treat leukemia or myeloma. In some embodiments, the engineered hematopoietic stem cells may be useful for treating a cellular deficiency associated with an autoimmune disease or condition or to treat an autoimmune disease or condition. In some embodiments, the autoimmune disease or condition is selected from the group consisting of: acute disseminated encephalomyelitis, acute hemorrhagic leukoencephalitis, Addison's disease, Agammaglobulinemia, alopecia areata, amyotrophic lateral sclerosis, ankylosing spondylitis, antiphospholipid syndrome, antisynthetase syndrome, atopic allergy, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, Balo disease, Balo concentric sclerosis, Bechets syndrome, Berger's disease, Bickerstaff's encephalitis, Blau syndrome, bullous pemphigoid, cancer, Castleman's disease, celiac disease, chronic inflammatory demyelinating polyneuropathy, chronic recurrent multifocal osteomyelitis, Churg-Strauss syndrome, cicatricial pemphigoid, Cogan syndrome, cold agglutinin disease, complement component 2 deficiency, cranial arteritis, CREST syndrome, Crohn's disease, Cushing's syndrome, cutaneous leukocytoclastic angiitis, Dego's disease, Dercum's disease, dermatitis herpetiformis, dermatomyositis, diabetes mellitus type 1, diffuse cutaneous systemic sclerosis, Dressler's syndrome, discoid lupus erythematosus, eczema, enthesitis-related arthritis, eosinophilic fasciitis, eosinophilic gastroenteritis, epidermolysis bullosa acquisita, erythema nodosum, essential mixed cryoglobulinemia, Evan's syndrome, firodysplasia ossificans progressiva, fibrosing aveolitis, gastritis, gastrointestinal pemphigoid, giant cell arteritis, glomerulonephritis, goodpasture's syndrome, Grave's disease, Guillain-Barre syndrome (GBS), Hashimoto's encephalitis, Hashimoto's thyroiditis, hemolytic anaemia, Henoch-Schonlein purpura, herpes gestationis, hypogammaglobulinemia, idiopathic inflammatory demyelinating disease, idiopathic pulmonary fibrosis, idiopathic thrombocytopenic purpura, IgA nephropathy, inclusion body myositis, inflammatory demyelinating polyneuropathy, interstitial cystitis, juvenile idiopathic arthritis, juvenile rheumatoid arthritis, Kawasaki's disease, Lambert-Eaton myasthenic syndrome, leukocytoclastic vasculitis, lichen planus, lichen sclerosus, linear IgA disease (LAD), Lou Gehrig's disease, lupoid hepatitis, lupus erythematosus, Majeed syndrome, Meniere's disease, microscopic polyangiitis, Miller-Fisher syndrome, mixed connective tissue disease, morphea, Mucha-Habermann disease, multiple sclerosis, myasthenia gravis, myositis, neuropyelitis optica, neuromyotonia, ocular cicatricial pemphigoid, opsoclonus myoclonus syndrome, ord thyroiditis, palindromic rheumatism, paraneoplastic cerebellar degeneration, paroxysmal nocturnal hemoglobinuria (PNH), Parry Romberg syndrome, Parsonnage-Turner syndrome, pars planitis, pemphigus, pemphigus vulgaris, permicious anemia, perivenous encephalomyelitis, POEMS syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatoid fever, sarcoidosis, Schmidt syndrome, Schnitzler syndrome, scleritis, scleroderma, Sjogren's syndrome, spondylarthropathy, Still's disease, stiff person syndrome, subacute bacterial endocarditis, Susac's syndrome, Sweet's syndrome, Sydenham chorea, sympathetic ophthalmia, Takayasu's arteritis, temporal arteritis, Tolosa-Hunt syndrome, transverse myelitis, ulcerative colitis, undifferentiated connective tissue disease, undifferentiated spondylarthropathy, vasculitis, vitiligo, or Wegener's granulomatosis.

In some embodiments, the donor of the therapeutic engineered cells or populations of cells provided herein is different from the patient (e.g., the recipient that is administered the therapeutic cells). In some embodiments, the pool of engineered cells or populations of cells does not include cells from the patient. In some embodiments, one or more of the donor subjects from which the pool of engineered cells or populations of cells is obtained are different from the patient. In some embodiments, the engineered cells or populations of cells differentiated into cell types as described herein may be used for subsequent transplantation or engraftment into subjects (e.g., recipients). In some embodiments, the population of engineered cells isolated from one or more individual donors (e.g., healthy donors) or engineered cells differentiated from iPSCs derived from one or more individual donors (e.g., healthy donors), are maintained in culture, in some cases expanded, prior to administration. In certain embodiments, the population of engineered cells is cryopreserved prior to administration.

In some embodiments, the engineered cells or populations of cells for use as described herein are T cells, and the pharmaceutical composition includes from about 2.0×10⁶ to about 2.0×10⁸ cells, such as but not limited to, primary T cells or T cells differentiated from engineered induced pluripotent stem cells. In some cases, the dose includes about 1.0×10¹ to about 2.5×10⁸ primary T cells described herein at a volume of about 10 mL to 50 mL. In several cases, the dose includes about 1.0×10⁵ to about 2.5×10⁸ primary T cells that have been described above at a volume of about 10 mL to 50 mL. In various cases, the dose includes about 1.0×10⁵ to about 2.5×10⁸ T cells differentiated from engineered induced pluripotent stem cells described herein at a volume of about 10 mL to 50 mL. In other cases, the dose is at a range that is lower than about 1.0×10¹ to about 2.5×10⁸ T cells, including primary T cells or T cells differentiated from engineered induced pluripotent stem cells. In yet other cases, the dose is at a range that is higher than about 1.0×10⁵ to about 2.5×10⁸ T cells, including primary T cells and T cells differentiated from engineered induced pluripotent stem cells. In some embodiments, the cells are engineered T cells (e.g., primary T cells or T cells differentiated from engineered induced pluripotent stem cells). The first dosage regimen can be administered to the subject at least 1 month, 2 months, 3 months, 4 months, 5 months, 6 months, 7 months, 8 months, 9 months, 10 months, 11 months, 12 months, 1-3 months, 1-6 months, 4-6 months, 3-9 months, 3-12 months, or more months apart from the second dosage regimen. In some embodiments, a subject is administered a plurality of dosage regimens during the course of a disease (e.g., cancer) and at least two of the dosage regimens comprise the same type of engineered T cells described herein. In other embodiments, at least two of the plurality of dosage regimens comprise different types of engineered T cells described herein.

In some embodiments, the therapeutic uses and methods provided herein further comprise administering one or more additional therapeutic agents to the subject. In some embodiments, the one or more additional therapeutics are selected from the group consisting of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a cell growth factor, a growth inhibitory agent, a cytotoxic agent, a hormonal therapy, a metabolic agent, a protein, a peptide, a pro-peptide, and any combination thereof. In some embodiments, the patient has been previously administered one or more therapeutic agents, such as any of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a cell growth factor, a growth inhibitory agent, a cytotoxic agent, a hormonal therapy, a metabolic agent, a protein, a peptide, a pro-peptide, or any combination thereof. For example, in some embodiments, the one or more additional therapeutic agents are selected from the group consisting of insulin, amylinomimetic(s), dopamine-2 agonist(s), DPP-4 inhibitor(s), metformin, alpha-glucosidase inhibitor(s), SGLT2 inhibitor(s), statins, GLP-1 receptor agonist(s), incretin, meglitinide(s), sulfonylureas, thiazolidinediones, nonsteroidal anti-inflammatory drugs (NSAIDs), antimalarial drugs, corticosteroids, azathioprine, mycophenolate, methotrexate, cyclosporine, voclosporin, leflunomide, belimumab, anifrolumab, abatacept, rituximab, vitamin D supplementation, dehydroepiandrosterone (DHEA), and any combination thereof. In some embodiments, the therapeutic uses and methods provided herein further comprise administering one or more immunosuppressive agents to the patient. In some embodiments, the therapeutic uses and methods provided herein further comprise administering an agent that triggers a kill switch to the patient. In some embodiments, the one or more additional therapeutic agents are administered via routes that include, but are not limited to, oral, intravenous, intracavitary, intratumoral, intraarterial, intravitreal, intramuscular, subcutaneous, parenteral, transmucosal, transdermal, ocular, topical, intraperitoneal, intracranial, intrapleural, and epidermal routes. In some embodiments, the engineered cells provided herein, populations of said engineered cells, or compositions thereof, and the one or more additional therapies are administered simultaneously or sequentially.

In some embodiments, the therapeutic uses and methods provided herein further comprise monitoring therapeutic efficacy, such as by monitoring whether the treated subject exhibits an elimination, reduction, or amelioration of the disease or condition, as described in Section V (“Methods of Monitoring a Sample”) above. In some embodiments, the therapeutic uses and methods provided herein further comprise monitoring the prophylactic efficacy of the method. Therapeutic or prophylactic efficacy in some embodiments is monitored by periodic assessment of treated subjects. In some embodiments, a subject is treated according to the therapeutic uses and methods provided herein until a desired suppression of one or more disease symptoms occurs.

### 2. Products

Further provided herein are methods of making therapeutic products, which, for example, may be used in any of the therapeutic uses and methods provided herein.

In one aspect, provided herein are methods for making a therapeutic cell product. In some embodiments, the methods comprise inserting a vector comprising a transgene (e.g., a kill switch) into a cell, such as any of the cells described herein. In some embodiments, the therapeutic protein is any protein suitable for the treatment of a disease or disorder. In some embodiments, the methods further comprise culturing the cell under conditions that allow for the survival and proliferation of the engineered cell or population of cells, and recovering the engineered cell or population of cells, for example, by separating, enriching, and/or purifying the engineered cell or population of cells from a larger pool of cells. In some embodiments, the therapeutic protein is selected from the group consisting of: enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases, recombinases, transposases, DNA polymerases, RNA polymerases, and reverse transcriptase.

In other aspects, provided herein are methods for making viral or virus-like particles for use in engineering the cell or population of cells derived from the sample as described herein.

In some embodiments, provided herein are methods for making a lentivirus particle for use in engineering the cell or population of cells derived from the sample as described herein. In some embodiments, the methods may comprise inserting nucleic acid(s) or vector(s) encoding a lentivirus particle into a cell, such as any of the cells provided herein. In some embodiments, the methods further comprise culturing the cell under conditions that allow for production of the lentivirus particle, and optionally recovering the lentivirus particle, for example, by separating, enriching, and/or purifying the lentivirus particle. The lentivirus particles can then be used to engineer a cell or population of cells as described herein to express one or more vectors comprising one or more transgenes.

In other embodiments, provided herein are methods for making a viral particle or virus-like particle comprising a fusogen for use in engineering the cell or population of cells derived from the sample as described herein. In some embodiments, the methods may comprise inserting nucleic acid(s) or vector(s) encoding a viral particle or virus-like particle and a fusogen into a cell, such as any of the cells provided herein. In some embodiments, the methods further comprise culturing the cell under conditions that allow for production of the viral particle or virus-like particle comprising the fusogen, and optionally recovering the viral particle or virus-like particle comprising the fusogen, for example, by separating, enriching, and/or purifying the viral particle or virus-like particle comprising the fusogen from the cell. The viral particles or virus-like particles can then be used to engineer a cell or population of cells as described herein to express one or more vectors comprising one or more transgenes.

In other embodiments, provided herein are methods for making an AAV particle for use in engineering the cell or population of cells derived from the sample as described herein. In some embodiments, the methods may comprise inserting nucleic acid(s) or vector(s) encoding an AAV particle into a cell, such as any of the cells provided herein. In some embodiments, the methods further comprise culturing the cell under conditions that allow for production of the AAV particle, and optionally recovering the AAV particle, for example, by separating, enriching, and/or purifying the AAV particle. The AAV particles can then be used to engineer a cell or population of cells as described herein to express one or more vectors comprising one or more transgenes.

In some embodiments, there is provided a method of selecting a cell population that is suitable for cell therapy, comprising detecting a proliferating cell expressing a detection agent in a population of cells, wherein the population of cells comprises a nucleic acid encoding the detection agent integrated at an endogenous proliferation gene locus and operably linked to the promoter of the endogenous proliferation gene. In some embodiments, the selected cell population comprises less than about 50%, about 45%, about 40%, about 35%, about 30%, about 25%, about 20%, about 15%, about 10%, about 9%, about 8%, about 7%, about 6%, about 5%, about 4%, about 3%, about 2%, or about 1% of cells expressing the detection agent. In some embodiments, the selected cell population comprises less than about 30-50%, about 20-40%, about 10-30%, about 5-20%, about 1-20%, about 1-15%, about 1-10%, about 1-9%, about 1-8%, about 1-7%, about 1-6%, about 1-5%, about 1-4%, about 1-3%, or about 1-2% of the total cell population. In some embodiments, the selected cell population comprises less than about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% of the total cell population.

In some embodiments there is provided a method of selecting a cell population that is suitable for cell therapy, comprising detecting an off-target cell expressing a detection agent in a population of cells, wherein the population of cells comprises a nucleic acid encoding the detection agent integrated at an off-target cell marker gene locus and operably linked to the promoter of the off-target cell marker gene. In some embodiments, the selected cell population comprises less than about 50%, about 45%, about 40%, about 35%, about 30%, about 25%, about 20%, about 15%, about 10%, about 9%, about 8%, about 7%, about 6%, about 5%, about 4%, about 3%, about 2%, or about 1% of cells expressing the detection agent. In some embodiments, the selected cell population comprises less than about 30-50%, 20-40%, 10-30%, 5-20%, 1-20%, 1-15%, 1-10%, about 1-9%, about 1-8%, about 1-7%, about 1-6%, about 1-5%, about 1-4%, about 1-3%, or about 1-2% of the total cell population. In some embodiments, the selected cell population comprises less than about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% of the total cell population.

In some embodiments, the methods further comprise administering the therapeutic product, i.e., the cells, the population of cells, or the composition thereof to a patient.

### 3. Kits

In some embodiments, there is provided a kit or an article of manufacture comprising any of the cells, populations of cells, or compositions thereof of the present disclosure. In some embodiments, the kit or the article of manufacture comprises instructions for use according to the methods and/or uses provided herein. In some embodiments, the kit further comprises an inducer.

In some embodiments, there is provided a kit or an article of manufacture comprising a modified cell provided herein, or populations and/or compositions thereof. In some embodiments, the kit or article of manufacture further comprises a package insert comprising instructions for use of the modified cell according to any of the methods or uses of the disclosure, such as a therapeutic method or use described herein. In some embodiments, the kit further comprises an inducer.

In some embodiments, the kit described herein further comprises an inducer, wherein the inducer is selected from the group consisting of rimiducid (AP1903), AP20187, rapamycin, 5-fluorocytosine, ganciclovir, CB 1954, 6-methylpurine deoxyriboside, fludarabine, indole-3-acetic acid (IAA), tetracycline, doxycycline, tamoxifen, cumate, FKCsA, abscisic acid (ABA), riboswitch, ecdysone, tryptophan, arabinose, isopropyl β-d-1-thiogalactopyranoside (IPTG), asunaprevir, grazoprevir, dTAG-13, lenalidomide, anti-MHC-I antibodies, anti-MHC-II antibodies, anti-CCR4 antibodies, anti-CD16 antibodies, anti-CD19 antibodies, anti-CD20 antibodies, anti-CD30 antibodies, anti-EGFR antibodies, anti-GD2 antibodies, anti-HER1 antibodies, anti-HER2 antibodies, anti-MUC1 antibodies, anti-PSMA antibodies, and anti-RQR8 antibodies. In some embodiments, the anti-CCR4 antibodies include, but are not limited to mogamulizumab and biosimilars thereof. In some embodiments, the anti-CD16 or anti-CD30 antibodies include, but are not limited to AFM13 and biosimilars thereof. In some embodiments, the anti-CD19 antibodies include, but are not limited to, tafasitamab, loncastuximab tesirine, MOR208, and biosimilars thereof. In some embodiments, In some embodiments, anti-CD20 antibodies include, but are not limited to, rituximab, obinutuzumab, ofatumumab, ublituximab, ocaratuzumab, rituximab-R11b, and biosimilars thereof. In some embodiments, the anti-EGFR (or anti-HER1) antibodies include, but are not limited to, tomuzotuximab, R05083945 (GA201), panitumumab, cetuximab, and biosimilars thereof. In some embodiments, the anti-GD2 antibodies include, but are not limited to, dinituximab and biosimilars thereof. In some embodiments, anti-HER2 antibodies include, but are not limited to, trastuzumab, pertuzumab, and biosimilars thereof. In some embodiments, the anti-GD2 antibodies include, but are not limited to, Hu14.18K322A, Hu14.18-IL2, Hu3F8, dinituximab, c.60C3-R11c, and biosimilars thereof.

In some embodiments, the kit described herein comprises a selection agent. In some embodiments, the selection agent is a kill switch. In some embodiments, the kill switch is selected from the group consisting of an inducible caspase 9 (iCasp9), cytosine deaminase (CDA), herpes simplex virus thymidine kinase (HSV-Tk), rapamycin-activated caspase 9 (rapaCasp9), chemically regulated-SH2-delivered inhibitory tail (CRASH-IT), nitroreductase (NTR), purine nucleoside phosphorylase (PNP), horseradish peroxidase, inducible MHC-I, inducible MHC-II, CCR4, CD16, CD19, CD20, CD30, EGFR, GD2, HER1, HER2, MUC1, PSMA, and RQR8. In some embodiments, the kill switch is iCasp9 or CDA. In some embodiments, the inducible MHC-I is selected from the group consisting of HLA-A, HLA-B, and HLA-C. In some embodiments, the inducible MHC-II is selected from the group consisting of HLA-DP, HLA-DQ, and HLA-DR. In some embodiments, the selection agent is a degron.

In some embodiments, the kit described herein comprises a detection agent, wherein the detection agent is a cell surface protein. In some embodiments, the cell surface protein is selected from the group consisting of EGFR fused to a His-tag, RQRB fused to a His-tag, and CD47 fused to a His-tag. In some embodiments, the detection agent is a fluorescent protein. In some embodiments, the fluorescent protein is selected from the group consisting of: green fluorescent protein (GFP), enhanced green fluorescent protein (EGFP), yellow fluorescent protein (YFP), enhanced yellow fluorescent protein (EYFP), blue fluorescent protein (BFP), enhanced blue fluorescent protein (EBFP), cyan fluorescent protein (CFP), enhanced cyan fluorescent protein (ECFP), superfolder GFP, superfolder YFP, orange fluorescent protein, red fluorescent protein, small ultrared fluorescent protein, FMN-binding fluorescent protein, dsRed, qFP611, Dronpa, TagRFP, KFP, EosFP, IrisFP, Dendra, Kaede, KikGrl, emerald fluorescent protein, Azami Green, mWasabi, TagGFP, TurboGFP, AcGFP, ZsGreen, and T-Sapphire. In some embodiments, the detection agent is associated with a barcode. In some embodiments, the detection agent is a blood-detectable biomarker. In some embodiments, the detection agent is a secreted protein. In some embodiments, the secreted protein is detectable in vitro. In some embodiments, in vitro detection comprises detecting the secreted protein in cell culture medium collected from a cell culture comprising the cell described herein. In some embodiments, the secreted protein is detectable ex vivo. In some embodiments, ex vivo detection comprises detecting the secreted protein in a blood sample taken from an individual administered the cells described herein.

In some embodiments, the kits described herein comprises a modified cell expressing an engineered receptor. In some embodiments, the engineered receptor is a chimeric antigen receptor (CAR). In some embodiments, the CAR comprises an extracellular antigen binding domain specifically recognizing a target antigen; a transmembrane domain; and an intracellular signaling domain. In some embodiments, the cell is an immune cell. In some embodiments, the immune cell is a T cell, a B cell, or a natural killer (NK) cell.

In some embodiments, the kit described herein comprises a cell, wherein the cell is a mammalian cell. In some embodiments, the mammalian cell is a human cell. In some embodiments, the cell is a stem cell-derived cell. In some embodiments, the stem cell-derived cell is derived from a cell selected from the group consisting of embryonic stem cell, induced pluripotent stem cell, multipotent stem cell, adult stem cell, hematopoietic stem cell, mesenchymal stem cell, endothelial stem cell, epithelial stem cell, adipose stem or progenitor cells, germline stem cells, lung stem or progenitor cells, mammary stem cells, olfactory adult stem cells, hair follicle stem cells, multipotent stem cells, amniotic stem cells, cord blood stem cells, neural stem, and progenitor cells. In some embodiments, the stem-cell derived cell is selected from the group consisting of a stem cell-derived beta cell, an alpha cell, and a delta cell. In some embodiments, the stem-cell derived cell is a stem cell-derived beta cell (SC-beta cell). In some embodiments, the stem-cell derived cell is a stem cell-derived beta islet cell. In some embodiments, the stem-cell derived cell is a stem cell-derived T cell. In some embodiments, the stem cell-derived T cell is selected from the group consisting of an ab T cell, dg T cell, helper/regulatory T cell, cytotoxic T cell, progenitor T cell (e.g., a progenitor T cell that is CD34+CD7+CD1a− or CD34+CD7+CD5+CD1a−), naive T cell, central memory T cell, effector T cell, terminal effector T cell, immature T cell, mature T cell, natural killer T cell, naive T cell, naive central memory T cell (TCM cell), effector memory T cell (TEM cell), and effector memory RA T cell (TEMRA cell). In some embodiments, the stem-cell derived cell is a stem cell-derived neural cell. In some embodiments, the stem cell-derived neural cell is selected from the group consisting of a glial cell, cerebral endothelial cell, neuron, ependymal cell, astrocyte, microglial cell, oligodendrocyte, and a Schwann cell. In some embodiments, the stem-cell derived cell is a stem cell-derived cardiac cell. In some embodiments, the stem cell-derived cardiac cell is selected from the group consisting of cardiomyocytes, nodal cardiomyocytes, conducting cardiomyocytes, working cardiomyocytes, cardiomyocyte precursors, cardiomyocyte progenitor cell, cardiac stem cell, and cardiac muscle cells.

In some embodiments, the kit described herein comprises modified cells comprises modifications that (i) increase expression of one or more tolerogenic factors, and (ii) reduce expression of one or more major histocompatibility complex (MHC) class I molecules and/or one or more MHC class II molecules, wherein the increased expression of (i) and the reduced expression of (ii) is relative to a cell of the same cell type that does not comprise the modifications. In some embodiments, the one or more of the modifications in (ii) reduce expression of: a. one or more MHC class I molecules; b. one or more MHC class II molecules; or c. one or more MHC class I molecules and one or more MHC class II molecules. In some embodiments, the one or more modifications reduce expression of one or more molecules selected from the group consisting of B2M, TAP I, NLRC5, CIITA, HLA-A, HLA-B, HLA-C, HLA-DP, HLA-DM, HLA-DOA, HLA-DOB, HLA-DQ, HLA-DR, RFX5, RFXANK, RFXAP, NFY-A, NFY-B and/or NFY-C and any combination thereof. In some embodiments, the engineered cell does not express one or more molecules selected from the group consisting of B2M, TAP I, NLRC5, CIITA, HLA-A, HLA-B, HLA-C, HLA-DP, HLA-DM, HLA-DOA, HLA-DOB, HLA-DQ, HLA-DR, and combinations thereof. In some embodiments, the one or more tolerogenic factors is selected from the group consisting of CD47, A20/TNFAIP3, C1-Inhibitor, CCL21, CCL22, CD16, CD16 Fc receptor, CD24, CD27, CD35, CD39, CD46, CD52, CD55, CD59, CD200, CR1, CTLA4-Ig, DUX4, FasL, H2-M3, HLA-C, HLA-E, HLA-E heavy chain, HLA-G, PD-L1, IDO1, IL-10, IL15-RF, IL-35, MANF, Mfge8, PD-1, PD-L1 or Serpinb9, and any combination thereof. In some embodiments, the one or more tolerogenic factors is selected from the group consisting of: a) CD47; b) HLA-E; c) CD24; d) PD-L1; e) CD46; f) CD55; f) CD59; h) CR1; i) MANF; j) A20/TNFAIP3; k) HLA-E and CD47; 1) CD24, CD47, PD-L1, and any combination thereof; m) HLA-E, CD24, CD47, and PD-L1, and any combination thereof; n) CD46, CD55, CD59, and CR1, and any combination thereof; o) HLA-E, CD46, CD55, CD59, and CR1, and any combination thereof; p) HLA-E, CD24, CD47, PDL1, CD46, CD55, CD59, and CR1, and any combination thereof; q) HLA-E and PDL1; r) HLA-E, PDL1, and A20/TNFAIP, and any combination thereof; sHLA-E, PDL1, and MANF, and any combination thereof; t) HLA-E, PDL1, A20/TNFAIP, and MANF, and any combination thereof; and u) CD47, PD-L1, HLA-E, HLA-G, CCL21, FASL, SERPINB9, CD200, MFGE8, and any combination thereof. In some embodiments, the engineered cell comprises modifications according to the following: (i) (a) reduces expression of MHC I and/or MHC II; (b) reduces expression of MIC-A and/or MIC-B; (c) increases expression of CD47, and optionally CD24 and PD-L1; and (d) increases expression of CD46, CD55, CD59 and CR1; (ii) (a) reduces expression of MHC class I molecule; (b) reduces expression of MIC-A and/or MIC-B; (c) reduces expression of TXNIP; (d) increases expression of PD-L1 and HLA-E; and (e) optionally increases expression of A20/TNFAIP3 and MANF; (iii) (a) increases expression of CCL21, PD-L1, FASL, SERPINB9, HLA-G, CD47, CD200, and MFGE8; and (b) reduces expression of a MICA and/or MICB; (iv) (a) reduces expression of MHC I and/or MHC II; and (b) increases expression of CD47; or (v) any of (i)-(iv) above further comprising modifications for increasing or decreasing expression of one or more additional genes, optionally reducing expression of B2M, TAP I, NLRC5, CIITA, HLA-A, HLA-B, HLA-C, HLA-DP, HLA-DM, HLA-DOA, HLA-DOB, HLA-DQ, HLA-DR, RFX5, RFXANK, RFXAP, NFY-A, NFY-B, NFY-C, CTLA-4, PD-1, IRF1, MIC-A, MIC-B, a protein that is involved in oxidative or ER stress, TRAC, TRB, CD142, ABO, CD38, PCDH11Y, NLGN4Y and/or RHD, further optionally wherein proteins that are is involved in oxidative or ER stress include thioredoxin-interacting protein (TXNIP), PKR-like ER kinase (PERK), inositol-requiring enzyme 1α (IRE1α), and DJ-1 (PARK7).

In some embodiments, the kit described herein comprises modified cells comprising modifications that (i) increase expression of one or more tolerogenic factors selected from the group consisting of CD47, PD-L1, HLA-E, HLA-G, CCL21, FASL, SERPINB9, CD200, MFGE8, and any combination thereof, and (ii) reduce expression of one or more major histocompatibility complex (MHC) class I molecules and/or one or more MHC class II molecules, wherein the increased expression of (i) and the reduced expression of (ii) is relative to a cell of the same cell type that does not comprise the modifications. In some embodiments, the modification(s) that increase expression comprise increased surface expression, and/or the modifications that reduce expression comprise reduced surface expression, optionally wherein there is no detectable surface expression. In some embodiments, the modification that increases expression of the one or more tolerogenic factors comprises an exogenous polynucleotide encoding the one or more tolerogenic factors. In some embodiments, the one or more tolerogenic factors comprises CD47. In some embodiments, the one or more tolerogenic factors is CD47 and the exogenous polynucleotide encoding CD47 encodes a sequence of amino acids having at least 85% identity to the amino acid sequence of SEQ ID NO: 2, and reduces innate immune killing of the engineered primary cell. In some embodiments, the exogenous polynucleotide encoding CD47 encodes a sequence set forth in SEQ ID NO: 2. In some embodiments, the exogenous polynucleotide encoding the one or more tolerogenic factors is operably linked to a promoter. In some embodiments, the promoter is a constitutive promoter. In some embodiments, the promoter is selected from the group consisting of the CAG promoter, the cytomegalovirus (CMV) promoter, the EF1α promoter, the PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein barr virus (EBV) promoter, the Rous sarcoma virus (RSV) promoter and the UBC promoter. In some embodiments, the exogenous polynucleotide encoding CD47 is integrated into the genome of the engineered primary cell. In some embodiments, the exogenous polynucleotide is a multicistronic vector encoding the one or more tolerogenic factors and an additional transgene encoding a second transgene. In some embodiments, the integration is by non-targeted insertion into the genome of the engineered primary cell, optionally by introduction of the exogenous polynucleotide into the cell using a lentiviral vector. In some embodiments, the integration is by targeted insertion into a target genomic locus of the cell. In some embodiments, the target genomic locus is a a B2M gene locus, a CIITA gene locus, a MICA locus, a MICB locus, a TRAC gene locus, or a TRBC gene locus. In some embodiments, the target genomic locus is selected from the group consisting of: a CCR5 gene locus, a CXCR4 gene locus, a PPPIR12C (also known as AAVS1) gene, an albumin gene locus, a SHS231 locus, a CLYBL gene locus, a ROSA26 gene locus, ABO gene locus, F3 gene locus, FUT1 gene locus, HMGB1 gene locus, KDM5D gene locus, LRP1 gene locus, RHD gene locus, ROSA26 gene locus, and SHS231 gene locus. In some embodiments, the modification that reduces expression of one or more MHC class I molecules reduces one or more MHC class I molecules protein expression. In some embodiments, the modification that reduces expression of one or more MHC class I molecules is a modification that reduces expression of B-2 microglobulin (B2M). In some embodiments, the modification that reduces expression of one or more MHC class I molecules comprises reduced mRNA expression of B2M. In some embodiments, the modification that reduces expression of one or more MHC class I molecules comprises reduced protein expression of B2M. In some embodiments, the modification eliminates B2M gene activity. In some embodiments, the modification comprises inactivation or disruption of both alleles of the B2M gene. In some embodiments, the modification comprises inactivation or disruption of all B2M coding sequences in the cell. In some embodiments, the inactivation or disruption comprises an indel in the B2M gene. In some embodiments, the modification is a frameshift mutation or a deletion of a contiguous stretch of genomic DNA of the B2M gene. In some embodiments, the B2M gene is knocked out. In some embodiments, the modification that reduces expression of one or more MHC class I molecules is a modification that reduces expression of an HLA-A protein, an HLA-B protein, or HLA-C protein, optionally wherein a gene encoding said HLA-A protein, an HLA-B protein, or HLA-C protein is knocked out. In some embodiments, the modification that reduces expression of one or more MHC class II molecules reduces one or more MHC class II molecules protein expression. In some embodiments, the modification that reduces expression of one or more MHC class II molecules is a modification that reduces expression of CIITA. In some embodiments, the modification that reduces expression of one or more MHC class II molecules comprises reduced mRNA expression of CIITA. In some embodiments, the modification that reduces expression of one or more MHC class II molecules comprises reduced protein expression of CIITA. In some embodiments, the modification eliminates CIITA gene activity. In some embodiments, the modification comprises inactivation or disruption of both alleles of the CIITA gene. In some embodiments, the modification comprises inactivation or disruption of all CIITA coding sequences in the cell. In some embodiments, the inactivation or disruption comprises an indel in the CIITA gene. In some embodiments, the modification that reduces expression of one or more MHC class II molecules is a modification that reduces expression of an HLA-DP protein, an HLA-DR protein, or HLA-DQ protein, optionally wherein a gene encoding said HLA-DP protein, an HLA-DR protein, or HLA-DQ protein is knocked out. In some embodiments, the modification that reduces expression of the one or more MHC class I molecules and/or the one or more MHC class II molecules is by a genome-modifying protein. In some embodiments, the genome-modifying protein is associated with gene editing by a sequence-specific nuclease, a CRISPR-associated transposase (CAST), prime editing, or Programmable Addition via Site-specific Targeting Elements (PASTE). In some embodiments, the modification by the genome-modifying protein is nuclease-mediated gene editing. In some embodiments, the nuclease-mediated gene editing is by a zinc finger nuclease (ZFN), a TAL-effector nuclease (TALEN), or a CRISPR-Cas combination that targets the B2M gene, optionally wherein the Cas is Cas9. In some embodiments, the nuclease-mediated gene editing is by a CRISPR-Cas combination and the CRISPR-Cas combination comprises a guide RNA (gRNA) having a targeting domain that is complementary to at least one target site of an endogenous gene for reducing the expression of the one or more MHC class I molecuels and/or one or more MHC class II molecules. In some embodiments, the CRISPR-Cas combination is a ribonucleoprotein (RNP) complex comprising the gRNA and a Cas protein. In some embodiments, the engineered primary cell is a human cell or an animal cell. In some embodiments, the engineered primary cell is a human cell. In some embodiments, the primary cell is a cell type that is exposed to the blood. In some embodiments, the engineered primary cell is a primary cell isolated from a donor subject. In some embodiments, the donor subject is healthy or is not suspected of having a disease or condition at the time the donor sample is obtained from the donor subject. In some embodiments, the engineered primary cell is selected from an islet cell, a beta islet cell, a pancreatic islet cell, an immune cell, a B cell, a T cell, a natural killer (NK) cell, a natural killer T (NKT) cell, a macrophage cell, an endothelial cell, a muscle cell, a cardiac muscle cell, a smooth muscle cell, a skeletal muscle cell, a dopaminergic neuron, a retinal pigmented epithelium cell, a hepatocyte, a thyroid cell, a skin cell, a glial progenitor cell, a neural cell, a cardiac cell, and a blood cell. In some embodiments, the engineered primary cell is an endothelial cell. In some embodiments, the engineered primary cell is an epithelial cell. In some embodiments, the engineered primary cell is a retinal pigmented epithelial cell. In some embodiments, the engineered primary cell is a T cell. In some embodiments, the engineered primary cell is an NK cell. In some embodiments, the engineered primary cell comprises a chimeric antigen receptor (CAR). In some embodiments, the engineered primary cell is an islet cell, optionally a beta islet cell. In some embodiments, the engineered primary cell is a hepatocyte. In some embodiments, the engineered primary cell is ABO blood group type O. In some embodiments, the engineered primary cell is Rhesus factor negative (Rh−).

In some embodiments, the kit described herein comprises modified cells capable of controlled killing of the engineered cell. In some embodiments, the engineered cell comprises a suicide gene or a suicide switch. In some embodiments, the suicide gene or the suicide switch induces controlled cell death in the presence of a drug or prodrug, or upon activation by a selective exogenous compound. In some embodiments, administration of an agent allows for depletion of an engineered cell of the population of engineered cells. In some embodiments, the agent recognizes the one or more tolerogenic factors on the surface of the engineered cell. In some embodiments, the engineered cell is engineered to express the one or more tolerogenic factors. In some embodiments, the one or more tolerogenic factors is CD47. In some embodiments, expression of a detection agent acts as a signal for administration of an exogenous kill switch directed against or specific to a tolerogenic agent. In some embodiments, the exogenous kill switch is an anti-CD47 antibody.

In some embodiments, the kits or articles of manufacture contain materials useful for clinical transplantation therapies, including cell therapies. In some embodiments, the kits or articles of manufacture contain a container and a label or package insert on or associated with the container. Suitable containers include, for example, bottles, vials, syringes, etc. (e.g., glass or plastic containers). Generally, the container holds a composition and may have a sterile access port (for example the container may be an intravenous solution bag or a vial having a stopper pierceable by a hypodermic injection needle). The label or package insert may indicate that the composition is used for treating a particular condition. The label or package insert may further comprise instructions for administering the composition to a patient, for example, instructions customarily included in commercial packages of therapeutic products such as information about the indications, usage, dosage, administration, contraindications and/or warnings concerning the use of such therapeutic products. In some embodiments, the kit or article of manufacture comprises a combination treatment as described herein.

### EXEMPLARY EMBODIMENTS

### First Set of Exemplary Embodiments

Embodiment 1. A cell comprising a nucleic acid encoding a selection agent or a detection agent integrated at an endogenous proliferation gene locus and operably linked to the promoter of the endogenous proliferation gene, wherein expression of the nucleic acid encoding the selection agent or the detection agent is regulated by the promoter of the endogenous proliferation gene and/or regulatory elements of the endogenous proliferation gene.

Embodiment 2. A cell comprising a nucleic acid encoding a selection agent and a detection agent integrated at an endogenous proliferation gene locus and operably linked to the promoter of the endogenous proliferation gene, wherein expression of the nucleic acid encoding the selection agent and the detection agent is regulated by the promoter of the endogenous proliferation gene and/or regulatory elements of the endogenous proliferation gene.

Embodiment 3. A cell comprising a nucleic acid encoding a first detection agent and a second detection agent, wherein the nucleic acid is integrated at an endogenous proliferation gene locus and operably linked to the promoter of the endogenous proliferation gene, and wherein expression of the nucleic acid encoding the first detection agent and the second detection agent is regulated by the promoter of the endogenous proliferation gene and/or regulatory elements of the endogenous proliferation gene.

Embodiment 4. A cell comprising a nucleic acid encoding a first selection agent and a second selection agent, wherein the nucleic acid is integrated at an endogenous proliferation gene locus and operably linked to the promoter of the endogenous proliferation gene, and wherein expression of the nucleic acid encoding the first selection agent and the second selection agent is regulated by the promoter of the endogenous proliferation gene and/or regulatory elements of the endogenous proliferation gene.

Embodiment 5. The cell of any one of Embodiments 1-4, wherein the first selection agent and the second selection agent are operably linked.

Embodiment 5a. The cell of Embodiment 5, wherein the first selection agent and the second selection agent are operably linked by an internal ribosome entry site.

Embodiment 5b. The cell of Embodiment 5, wherein the first selection agent and the second selection agent are operably linked by a self-cleaving peptide.

Embodiment 6. A cell comprising a first nucleic acid encoding a selection agent and a second nucleic acid encoding a detection agent, wherein the first nucleic acid is integrated at a first endogenous proliferation gene locus and operably linked to the promoter of the first endogenous proliferation gene, wherein expression of the first nucleic acid encoding the selection agent is regulated by the promoter of the first endogenous proliferation gene and/or regulatory elements of the first endogenous proliferation gene, wherein the second nucleic acid is integrated at a second endogenous proliferation gene locus and operably linked to the promoter of the second endogenous proliferation gene, and wherein expression of the second nucleic acid encoding the detection agent is regulated by the promoter of the second endogenous proliferation gene and/or regulatory elements of the endogenous proliferation gene.

Embodiment 7. A cell comprising a first nucleic acid encoding a first selection agent and a second nucleic acid encoding a second selection agent, wherein the first nucleic acid is integrated at a first endogenous proliferation gene locus and operably linked to the promoter of the first endogenous proliferation gene, wherein expression of the first nucleic acid encoding the first selection agent is regulated by the promoter of the first endogenous proliferation gene and/or regulatory elements of the first endogenous proliferation gene, wherein the second nucleic acid is integrated at a second endogenous proliferation gene locus and operably linked to the promoter of the second endogenous proliferation gene, and wherein expression of the second nucleic acid encoding the second selection agent is regulated by the promoter of the second endogenous proliferation gene and/or regulatory elements of the endogenous proliferation gene.

Embodiment 8. A cell comprising a first nucleic acid encoding a first detection agent and a second nucleic acid encoding a second detection agent, wherein the first nucleic acid is integrated at a first endogenous proliferation gene locus and operably linked to the promoter of the first endogenous proliferation gene, wherein expression of the first nucleic acid encoding the first detection agent is regulated by the promoter of the first endogenous proliferation gene and/or regulatory elements of the first endogenous proliferation gene, wherein the second nucleic acid is integrated at a second endogenous proliferation gene locus and operably linked to the promoter of the endogenous proliferation gene, and wherein expression of the second nucleic acid encoding the second detection agent is regulated by the promoter of the second endogenous proliferation gene and/or regulatory elements of the second endogenous proliferation gene.

Embodiment 9. The cell of any one of Embodiments 6-8, wherein the first endogenous proliferation gene and/or the second endogenous proliferation gene are expressed in a cell that is proliferating.

Embodiment 10. The cell of any one of Embodiments 6-9, wherein the first endogenous proliferation gene and/or the second endogenous proliferation gene are expressed in a cell that is included in a population of therapeutic cells.

Embodiment 11. The cell of any one of Embodiments 6-10, wherein the first endogenous proliferation gene and/or the second endogenous proliferation gene are expressed in a cell that is included in a population of therapeutic cells but is not a therapeutic cell.

Embodiment 12. The cell of any one of Embodiments 1-11, wherein the cell is a pluripotent cell, a progenitor cell, or a differentiated cell that is proliferating.

Embodiment 13. The cell of any one of Embodiments 6-12, wherein the first endogenous proliferation gene and the second endogenous proliferation gene are the same, and the first and second nucleic acids are integrated at different alleles.

Embodiment 14. The cell of any one of Embodiments 6-12, wherein the first endogenous proliferation gene and the second endogenous proliferation gene are different.

Embodiment 15. The cell of any one of Embodiments 1-14, wherein the endogenous proliferation gene is expressed at a higher level from day 0 to about day 5 of differentiation, compared to expression of the endogenous proliferation gene at day 18 of differentiation, or wherein the endogenous proliferation gene is highly expressed from day 1 to about day 5 of differentiation.

Embodiment 16. The cell of any one of Embodiments 1-15, wherein the endogenous proliferation gene is selected from the group consisting of AURKB, CDK1, CDC20, RRM2, BIRC5, TOP2A, PTTG1, CCNB1, TPX2, KIF11, SPC25, CENPK, SMC4, TYMS, H2AZ1, TMSB15A, CENPF, and MKI67.

Embodiment 16a. The cell of any one of Embodiments 1-16, wherein the endogenous proliferation gene is selected from the group consisting of AURKB, RRM2, TOP2A, PTTG1, CCNB1, SPC25, CENPK, SMC4, TYMS, H2AZ1, TMSB15A, CENPF, and MKI67.

Embodiment 16b. The cell of any one of Embodiments 1-16a, wherein the endogenous proliferation gene is AURKB.

Embodiment 16c. The cell of any one of Embodiments 1-16a, wherein the endogenous proliferation gene is RRM2.

Embodiment 16d. The cell of any one of Embodiments 1-16a, wherein the endogenous proliferation gene is TOP2A.

Embodiment 16e. The cell of any one of Embodiments 1-16a, wherein the endogenous proliferation gene is PTTG1.

Embodiment 16f. The cell of any one of Embodiments 1-16a, wherein the endogenous proliferation gene is CCNB1.

Embodiment 16g. The cell of any one of Embodiments 1-16a, wherein the endogenous proliferation gene is SPC25.

Embodiment 16 h. The cell of any one of Embodiments 1-16a, wherein the endogenous proliferation gene is CENPK.

Embodiment 16i. The cell of any one of Embodiments 1-16a, wherein the endogenous proliferation gene is SMC4.

Embodiment 16j. The cell of any one of Embodiments 1-16a, wherein the endogenous proliferation gene is TYMS.

Embodiment 16k. The cell of any one of Embodiments 1-16a, wherein the endogenous proliferation gene is H2AZ1.

Embodiment 161. The cell of any one of Embodiments 1-16a, wherein the endogenous proliferation gene is TMSB15A.

Embodiment 16m. The cell of any one of Embodiments 1-16a, wherein the endogenous proliferation gene is CENPF.

Embodiment 16n. The cell of any one of Embodiments 1-16a, wherein the endogenous proliferation gene is MKI67.

Embodiment 17. The cell of any one of Embodiments 1-16n, wherein the nucleic acid encoding the selection agent or the detection agent is integrated in a 5′ UTR or a 3′ UTR of the endogenous proliferation gene.

Embodiment 17a. The cell of any one of Embodiments 1-17, wherein the nucleic acid encoding the selection agent or the detection agent is integrated in a 5′ UTR or a 3′ UTR of AURKB.

Embodiment 17b. The cell of any one of Embodiments 1-17, wherein the nucleic acid encoding the selection agent or the detection agent is integrated in a 5′ UTR or a 3′ UTR of CDC20.

Embodiment 17c. The cell of any one of Embodiments 1-17, wherein the nucleic acid encoding the selection agent or the detection agent is integrated in a 5′ UTR or a 3′ UTR of CDK1.

Embodiment 17d. The cell of any one of Embodiments 1-17, wherein the nucleic acid encoding the selection agent or the detection agent is integrated in a 5′ UTR or a 3′ UTR of RRM2.

Embodiment 17e. The cell of any one of Embodiments 1-17, wherein the nucleic acid encoding the selection agent or the detection agent is integrated in a 5′ UTR or a 3′ UTR of BIRC5.

Embodiment 17f. The cell of any one of Embodiments 1-17, wherein the nucleic acid encoding the selection agent or the detection agent is integrated in a 5′ UTR or a 3′ UTR of TOP2A.

Embodiment 17g. The cell of any one of Embodiments 1-17, wherein the nucleic acid encoding the selection agent or the detection agent is integrated in a 5′ UTR or a 3′ UTR of PTTG1.

Embodiment 17 h. The cell of any one of Embodiments 1-17, wherein the nucleic acid encoding the selection agent or the detection agent is integrated in a 5′ UTR or a 3′ UTR of CCNB1.

Embodiment 17i. The cell of any one of Embodiments 1-17, wherein the nucleic acid encoding the selection agent or the detection agent is integrated in a 5′ UTR or a 3′ UTR of TPX2.

Embodiment 17j. The cell of any one of Embodiments 1-17, wherein the nucleic acid encoding the selection agent or the detection agent is integrated in a 5′ UTR or a 3′ UTR of KIF11.

Embodiment 17k. The cell of any one of Embodiments 1-17, wherein the nucleic acid encoding the selection agent or the detection agent is integrated in a 5′ UTR or a 3′ UTR of SPC25.

Embodiment 171. The cell of any one of Embodiments 1-17, wherein the nucleic acid encoding the selection agent or the detection agent is integrated in a 5′ UTR or a 3′ UTR of CENPK.

Embodiment 17m. The cell of any one of Embodiments 1-17, wherein the nucleic acid encoding the selection agent or the detection agent is integrated in a 5′ UTR or a 3′ UTR of SMC4.

Embodiment 17n. The cell of any one of Embodiments 1-17, wherein the nucleic acid encoding the selection agent or the detection agent is integrated in a 5′ UTR or a 3′ UTR of TYMS.

Embodiment 17o. The cell of any one of Embodiments 1-17, wherein the nucleic acid encoding the selection agent or the detection agent is integrated in a 5′ UTR or a 3′ UTR of H2AZ1.

Embodiment 17p. The cell of any one of Embodiments 1-17, wherein the nucleic acid encoding the selection agent or the detection agent is integrated in a 5′ UTR or a 3′ UTR of TMSB15A.

Embodiment 17q. The cell of any one of Embodiments 1-17, wherein the nucleic acid encoding the selection agent or the detection agent is integrated in a 5′ UTR or a 3′ UTR of CENPF.

Embodiment 17r. The cell of any one of Embodiments 1-17, wherein the nucleic acid encoding the selection agent or the detection agent is integrated in a 5′ UTR or a 3′ UTR of MKI67.

Embodiment 18. The cell of any one of Embodiments 1-16n, wherein the nucleic acid encoding the selection agent or the detection agent is integrated in an exon or an intron of the endogenous proliferation gene.

Embodiment 18a. The cell of Embodiment 18, wherein the nucleic acid encoding the selection agent or the detection agent is integrated in an exon of AURKB, optionally wherein the exon is exon 1, 2, 3, 4, 5, 6, 7, 8, or 9.

Embodiment 18b. The cell of Embodiment 18, wherein the nucleic acid encoding the selection agent or the detection agent is integrated in an intron of AURKB, optionally wherein the intron is intron 1, 2, 3, 4, 5, 6, 7, or 8.

Embodiment 18c. The cell of Embodiment 18, wherein the nucleic acid encoding the selection agent or the detection agent is integrated in an exon of CDC20, optionally wherein the exon is exon 1, 2, 3, 4, 5, 6, 7, 8, 9, 10, or 11.

Embodiment 18d. The cell of Embodiment 18, wherein the nucleic acid encoding the selection agent or the detection agent is integrated in an intron of CDC20, optionally wherein the intron is intron 1, 2, 3, 4, 5, 6, 7, 8, 9, or 10.

Embodiment 18e. The cell of Embodiment 18, wherein the nucleic acid encoding the selection agent or the detection agent is integrated in an exon of RRM2, optionally wherein the exon is exon 1, 2, 3, 4, 5, 6, 7, 8, 9, or 10.

Embodiment 18f. The cell of Embodiment 18, wherein the nucleic acid encoding the selection agent or the detection agent is integrated in an intron of RRM2, optionally wherein the intron is intron 1, 2, 3, 4, 5, 6, 7, 8, or 9.

Embodiment 18g. The cell of Embodiment 18, wherein the nucleic acid encoding the selection agent or the detection agent is integrated in an exon of CDK1, optionally wherein the exon is exon 1, 2, 3, 4, 5, 6, 7, or 8.

Embodiment 18 h. The cell of Embodiment 18, wherein the nucleic acid encoding the selection agent or the detection agent is integrated in an intron of CDK1, optionally wherein the intron is intron 1, 2, 3, 4, 5, 6, or 7.

Embodiment 18i. The cell of Embodiment 18, wherein the nucleic acid encoding the selection agent or the detection agent is integrated in an exon of BIRC5, optionally wherein the exon is exon 1, 2, 3, or 4.

Embodiment 18j. The cell of Embodiment 18, wherein the nucleic acid encoding the selection agent or the detection agent is integrated in an intron of BIRC5, optionally wherein the intron is intron 1, 2, or 3.

Embodiment 18k. The cell of Embodiment 18, wherein the nucleic acid encoding the selection agent or the detection agent is integrated in an exon of TOP2A, optionally wherein the exon is exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35.

Embodiment 181. The cell of Embodiment 18, wherein the nucleic acid encoding the selection agent or the detection agent is integrated in an intron of TOP2A, optionally wherein the intron is intron 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34.

Embodiment 18m. The cell of Embodiment 18, wherein the nucleic acid encoding the selection agent or the detection agent is integrated in an exon of PTTG1, optionally wherein the exon is exon 1, 2, 3, 4, 5, or 6.

Embodiment 18n. The cell of Embodiment 18, wherein the nucleic acid encoding the selection agent or the detection agent is integrated in an intron of PTTG1, optionally wherein the intron is intron 1, 2, 3, 4, or 5.

Embodiment 18o. The cell of Embodiment 18, wherein the nucleic acid encoding the selection agent or the detection agent is integrated in an exon of CCNB1, optionally wherein the exon is exon 1, 2, 3, 4, 5, 6, 7, 8, or 9.

Embodiment 18p. The cell of Embodiment 18, wherein the nucleic acid encoding the selection agent or the detection agent is integrated in an intron of CCNB1, optionally wherein the intron is intron 1, 2, 3, 4, 5, 6, 7, or 8.

Embodiment 18q. The cell of Embodiment 18, wherein the nucleic acid encoding the selection agent or the detection agent is integrated in an exon of TPX2, optionally wherein the exon is exon 1, 2, 3, 4, 5, 6, 7, 8, 9, 10, 11, 12, 13, 14, 15, 16, 17, or 18.

Embodiment 18r. The cell of Embodiment 18, wherein the nucleic acid encoding the selection agent or the detection agent is integrated in an intron of TPX2, optionally wherein the intron is intron 1, 2, 3, 4, 5, 6, 7, 8, 9, 10, 11, 12, 13, 14, 15, 16, or 17.

Embodiment 18s. The cell of Embodiment 18, wherein the nucleic acid encoding the selection agent or the detection agent is integrated in an exon of KIF11, optionally wherein the exonis exon 1, 2, 3, 4, 5, 6, 7, 8, 9, 10, 11, 12, 13, 14, 15, 16, 17, 18, 19, 20, 21, or 22.

Embodiment 18t. The cell of Embodiment 18, wherein the nucleic acid encoding the selection agent or the detection agent is integrated in an intron of KIF11, optionally wherein the intron is intron 1, 2, 3, 4, 5, 6, 7, 8, 9, 10, 11, 12, 13, 14, 15, 16, 17, 18, 19, 20, or 21.

Embodiment 18u. The cell of Embodiment 18, wherein the nucleic acid encoding the selection agent or the detection agent is integrated in an exon of SPC25, optionally wherein the exon is exon 1, 2, 3, 4, 5, 6, or 7.

Embodiment 18v. The cell of Embodiment 18, wherein the nucleic acid encoding the selection agent or the detection agent is integrated in an intron of SPC25, optionally wherein the intron is intron 1, 2, 3, 4, 5, or 6.

Embodiment 18w. The cell of Embodiment 18, wherein the nucleic acid encoding the selection agent or the detection agent is integrated in an exon of CENPK, optionally wherein the exon is exon 1, 2, 3, 4, 5, 6, 7, 8, 9, 10, or 11.

Embodiment 18x. The cell of Embodiment 18, wherein the nucleic acid encoding the selection agent or the detection agent is integrated in an intron of CENPK, optionally wherein the intron is intron 1, 2, 3, 4, 5, 6, 7, 8, 9, or 10.

Embodiment 18y. The cell of Embodiment 18, wherein the nucleic acid encoding the selection agent or the detection agent is integrated in an exon of SMC4, optionally wherein the exon is exon 1, 2, 3, 4, 5, 6, 7, 8, 9, 10, 11, 12, 13, 14, 15, 16, 17, 18, 19, 20, 21, 22, 23, or 24.

Embodiment 18z. The cell of Embodiment 18, wherein the nucleic acid encoding the selection agent or the detection agent is integrated in an intron of SMC4, optionally wherein the intron is intron 1, 2, 3, 4, 5, 6, 7, 8, 9, 10, 11, 12, 13, 14, 15, 16, 17, 18, 19, 20, 21, 22, or 23.

Embodiment 18aa. The cell of Embodiment 18, wherein the nucleic acid encoding the selection agent or the detection agent is integrated in an exon of TYMS, optionally wherein the exon is exon 1, 2, 3, 4, 5, 6, or 7.

Embodiment 18bb. The cell of Embodiment 18, wherein the nucleic acid encoding the selection agent or the detection agent is integrated in an intron of TYMS, optionally wherein the intron is intron 1, 2, 3, 4, 5, or 6.

Embodiment 18cc. The cell of Embodiment 18, wherein the nucleic acid encoding the selection agent or the detection agent is integrated in an exon of H2AZ1, optionally wherein the exon is exon 1, 2, 3, 4, or 5.

Embodiment 18dd. The cell of Embodiment 18, wherein the nucleic acid encoding the selection agent or the detection agent is integrated in an intron of H2AZ1, optionally wherein the intron is intron 1, 2, 3, or 4.

Embodiment 18ee. The cell of Embodiment 18, wherein the nucleic acid encoding the selection agent or the detection agent is integrated in an exon of TMSB15A, optionally wherein the exon is exon 1, 2, or 3.

Embodiment 18ff. The cell of Embodiment 18, wherein the nucleic acid encoding the selection agent or the detection agent is integrated in an intron of TMSB15A, optionally wherein the intron is intron 1 or 2.

Embodiment 18gg. The cell of Embodiment 18, wherein the nucleic acid encoding the selection agent or the detection agent is integrated in an exon of CENPF, optionally wherein the exon is exon 1, 2, 3, 4, 5, 6, 7, 8, 9, 10, 11, 12, 13, 14, 15, 16, 17, 18, 19, or 20.

Embodiment 18hh. The cell of Embodiment 18, wherein the nucleic acid encoding the selection agent or the detection agent is integrated in an intron of CENPF, optionally wherein the intron is intron 1, 2, 3, 4, 5, 6, 7, 8, 9, 10, 11, 12, 13, 14, 15, 16, 17, 18, or 19.

Embodiment 18ii. The cell of Embodiment 18, wherein the nucleic acid encoding the selection agent or the detection agent is integrated in an exon of MKI67, optionally wherein the exon is exon 1, 2, 3, 4, 5, 6, 7, 8, 9, 10, 11, 12, 13, 14, or 15.

Embodiment 18jj. The cell of Embodiment 18, wherein the nucleic acid encoding the selection agent or the detection agent is integrated in an intron of MKI67, optionally wherein the intron is intron 1, 2, 3, 4, 5, 6, 7, 8, 9, 10, 11, 12, 13, or 14.

Embodiment 19. The cell of any one of Embodiments 1-18jj, wherein following integration, the endogenous proliferation gene locus comprises i) nucleic acid encoding the endogenous proliferation gene, ii) an internal ribosomal entry site or a self-cleaving RNA site, and iii) the nucleic acid encoding the selection agent or a detection agent.

Embodiment 20. The cell of any one of Embodiments 1-19, wherein the selection agent or the detection agent is expressed in proliferating cells.

Embodiment 21. The cell of any one of Embodiments 1-20, wherein the selection agent or the detection agent is not expressed in non-proliferating cells.

Embodiment 22. The cell of any one of Embodiments 1-21, wherein the selection agent or the detection agent is expressed in partially differentiated cells.

Embodiment 23. The cell of any one of Embodiments 1-22, wherein the selection agent or the detection agent is expressed in an intermediate cell type made during differentiation.

Embodiment 24. The cell of any one of Embodiments 1-23, wherein the selection agent or the detection agent is not expressed in differentiated cells.

Embodiment 25. The cell of any one of Embodiments 1-24, wherein the selection agent or the detection agent is not expressed in a therapeutic cell.

Embodiment 26. The cell of any one of Embodiments 1-25, wherein the cell is selected from the group consisting of a pluripotent stem cell, an induced pluripotent stem cell, a primary cell, a progenitor cell, an embryonic stem cell, a hematopoietic stem cell, a mesenchymal stem cell, an epithelial stem cell, a germline stem cell, a mammary stem cell, an olfactory adult stem cell, a hair follicle stem cell, a multipotent stem cell, an amniotic stem cell, a cord blood stem cell, a neural stem cell, a somatic stem cell, a totipotent stem cell, a fibroblast, a monocytic precursor, an exocrine cell, a pancreatic progenitor, an endocrine progenitor, a hepatoblast, a myoblast, a preadipocyte, a chondrocyte, a bone cell, a synovial cell, a tendon cell, a ligament cell, a meniscus cell, an adipose cell, a dendritic cell, a natural killer cell, a muscle cell, an erythroid-megakaryocytic cell, an eosinophil, an islet beta cell, a neuron, a cardiomyocyte, a blood cell, an exocrine progenitor, a ductal cell, an acinar cell, an alpha cell, a beta cell, a delta cell, a cholangiocyte, a brown adipocyte, a cardiac muscle cell, a PP cell, an epidermal keratinocyte, an epithelial cell, a germ cell, a skeletal joint synovium cell, a periosteum cell, a bone cell, a perichondrium cell, a pericardium cell, a meningeal cell, a keratinocyte precursor cell, a keratinocyte stem cell, a pericyte, a glial cell, an ependymal cell, a serosal cell, a heart cell, a brain cell, a spinal cord cell, a lung cell, a pancreas cell, a bladder cell, a bone marrow cell, a spleen cell, an intestine cell, a stomach cell, an islet cell, a pancreatic islet cell, an immune cell, a B cell, a T cell, a natural killer (NK) cell, a natural killer T (NKT) cell, a macrophage, an endothelial cell, a muscle cell, a smooth muscle cell, a skeletal muscle cell, a dopaminergic neuron, a retinal pigmented epithelium cell, an optic cell, a hepatocyte, a thyroid cell, a skin cell, a glial progenitor cell, a neural cell, a stem cell, an endothelial stem cell, an adipose stem cell, an adipose progenitor cell, a lung stem cell, a lung progenitor cell, a neural progenitor cell, a T cell, a CAR-T cell, a CD14+ cell, a dendritic cell, a PBMC cell, an exocrine secretory epithelial cell, a thyroid epithelial cell, a keratinizing epithelial cell, a gall bladder epithelial cell, a surface epithelial cell, a kidney cell, a primary T cell, a CD3+ T cell, a CD4+ T cell, a CD8+ T cell, a naive T cell, a regulatory T (Treg) cell, a non-regulatory T cell, a Th1 cell, a Th2 cell, a Th9 cell, a Th17 cell, a T-follicular helper (Tfh) cell, a cytotoxic T lymphocyte (CTL), an effector T (Teff) cell, a central memory T (Tcm) cell, an effector memory T (Tem) cell, a TEMRA cells, a tissue-resident memory (Trm) cell, a virtual memory T cell, an innate memory T cell, a memory stem cell (Tsc), a T6 T cell, a helper T cell, a central memory T cell, an effector memory T cell, an effector memory RA T cell, a tissue infiltrating lymphocyte, a capillary endothelial cell, a vascular endothelial cell, an aortic endothelial cell, an arterial endothelial cell, a venous endothelial cell, a renal endothelial cell, a brain endothelial cell, a liver endothelial cell, a gamma cell, an epsilon cell, hematopoietic progenitor cell, a nodal cardiomyocyte, a conducting cardiomyocyte, a working cardiomyocyte, a cardiomyocyte precursor cell, a cardiomyocyte progenitor cell, a cardiac stem cell, an atrial cardiac stem cell, a ventricular cardiac stem cell, an epicardial cell, a vascular endothelial cell, an endocardial endothelial cell, a cardiac valve interstitial cell, a cardiac pacemaker cell, a neuroectodermal cell, a neuronal cell, a neuroendocrine cell, a cholinergic cell, a serotonergic (5-HT) cell, a glutamatergic cell, a GABAergic cell, an adrenergic cell, a noradrenergic cell, a sympathetic neuronal cell, a parasympathetic neuronal cell, a sympathetic peripheral neuronal cell, an astrocyte, an oligodendrocyte, an ependymal cell, a radial glia cell, a Schwann cell, a cerebral endothelial cells (ECs), a neuronal stem cell, a neuronal progenitor cell, a white blood cell, a red blood cell, a platelet cell, a tumor cell, an enterochromaffin cell, a mesenchymal fibroblast, an osteoblast, a stromal cell, and any combination thereof. 26a. The cell of Embodiment 26, wherein the cell is selected from the group consisting of a pancreatic islet cell, an alpha cell, a beta cell, a gamma cell, a delta cell, an epsilon cell, a T cell, a neuron, a glial cell, a cardiomyocyte, a retinal pigmented epithelial cell, a hematopoietic progenitor cell, a natural killer cell, an endothelial cell, and a lung cell.

Embodiment 27. A cell comprising a nucleic acid encoding a selection agent or a detection agent integrated at an off-target cell type gene locus and operably linked to the promoter of the off-target cell marker gene, wherein expression of the nucleic acid encoding the selection agent or the detection agent is regulated by the promoter of the off-target cell marker gene and/or regulatory elements of the off-target cell marker gene.

Embodiment 28. A cell comprising a nucleic acid encoding a selection agent and a detection agent integrated at an off-target cell type gene locus and operably linked to the promoter of the off-target cell type gene, wherein expression of the nucleic acid encoding the selection agent and the detection agent is regulated by the promoter of the off-target cell type gene and/or regulatory elements of the off-target cell type gene.

Embodiment 29. A cell comprising a nucleic acid encoding a first detection agent and a second detection agent, wherein the nucleic acid is integrated at an off-target cell type gene locus and operably linked to the promoter of the off-target cell type gene, and wherein expression of the nucleic acid encoding the first detection agent and the second detection agent is regulated by the promoter of the off-target cell type gene and/or regulatory elements of the off-target cell type gene.

Embodiment 30. A cell comprising a nucleic acid encoding a first selection agent and a second selection agent, wherein the nucleic acid is integrated at an off-target cell type gene locus and operably linked to the promoter of the off-target cell type gene, and wherein expression of the nucleic acid encoding the first selection agent and the second selection agent is regulated by the promoter of the off-target cell type gene and/or regulatory elements of the off-target cell type gene.

Embodiment 31. The cell of any one of Embodiments 27-30, wherein the first selection agent and the second selection agent are operably linked.

Embodiment 31a. The cell of Embodiment 31, wherein the first selection agent and the second selection agent are operably linked by an internal ribosome entry site.

Embodiment 31b. The cell of Embodiment 31, wherein the first selection agent and the second selection agent are operably linked by a self-cleaving peptide.

Embodiment 32. A cell comprising a first nucleic acid encoding a selection agent and a second nucleic acid encoding a detection agent, wherein the first nucleic acid is integrated at a first off-target cell type gene locus and operably linked to the promoter of the first off-target cell type gene, wherein expression of the first nucleic acid encoding the selection agent is regulated by the promoter of the first off-target cell type gene and/or regulatory elements of the first off-target cell type gene, wherein the second nucleic acid is integrated at a second off-target cell type gene locus and operably linked to the promoter of the second off-target cell type gene, and wherein expression of the second nucleic acid encoding the detection agent is regulated by the promoter of the second off-target cell type gene and/or regulatory elements of the off-target cell type gene.

Embodiment 33. A cell comprising a first nucleic acid encoding a first selection agent and a second nucleic acid encoding a second selection agent, wherein the first nucleic acid is integrated at a first off-target cell type gene locus and operably linked to the promoter of the first off-target cell type gene, wherein expression of the first nucleic acid encoding the first selection agent is regulated by the promoter of the first off-target cell type gene and/or regulatory elements of the first off-target cell type gene, wherein the second nucleic acid is integrated at a second off-target cell type gene locus and operably linked to the promoter of the second off-target cell type gene, and wherein expression of the second nucleic acid encoding the second selection agent is regulated by the promoter of the second off-target cell type gene and/or regulatory elements of the off-target cell type gene.

Embodiment 34. A cell comprising a first nucleic acid encoding a first detection agent and a second nucleic acid encoding a second detection agent, wherein the first nucleic acid is integrated at a first off-target cell type gene locus and operably linked to the promoter of the first off-target cell type gene, wherein expression of the first nucleic acid encoding the first detection agent is regulated by the promoter of the first off-target cell type gene and/or regulatory elements of the first off-target cell type gene, wherein the second nucleic acid is integrated at a second off-target cell type gene locus and operably linked to the promoter of the off-target cell type gene, and wherein expression of the second nucleic acid encoding the second detection agent is regulated by the promoter of the second off-target cell type gene and/or regulatory elements of the second off-target cell type gene.

Embodiment 35. The cell of any one of Embodiments 32-34, wherein the first off-target cell type gene and/or the second off-target cell type gene are expressed in a cell that is included in a population of therapeutic cells.

Embodiment 36. The cell of any one of Embodiments 32-35, wherein the first off-target cell type gene and/or the second off-target cell type gene are expressed in a cell that is included in a population of therapeutic cells but is not a therapeutic cell.

Embodiment 37. The cell of any one of Embodiments 27-36, wherein the cell is an off-target cell.

Embodiment 38. The cell of any one of Embodiments 32-37, wherein the first off-target cell type gene and the second off-target cell type gene are the same, and the first and second nucleic acids are integrated at different alleles.

Embodiment 39. The cell of any one of Embodiments 32-37, wherein the first off-target cell type gene and the second off-target cell type gene are different.

Embodiment 40. The cell of any one of Embodiments 27-39, wherein the off-target cell marker gene is selected from the group consisting of a pluripotent stem cell marker gene, an induced pluripotent stem cell marker gene, a primary cell marker gene, a progenitor cell marker gene, an embryonic stem cell marker gene, a hematopoietic stem cell marker gene, a mesenchymal stem cell marker gene, an epithelial stem cell marker gene, a germline stem cell marker gene, a mammary stem cell marker gene, an olfactory adult stem cell marker gene, a hair follicle stem cell marker gene, a multipotent stem cell marker gene, an amniotic stem cell marker gene, a cord blood stem cell marker gene, a neural stem cell marker gene, a somatic stem cell marker gene, a totipotent stem cell marker gene, a fibroblast marker gene, a monocytic precursor marker gene, an exocrine cell marker gene, a pancreatic progenitor marker gene, an endocrine progenitor marker gene, a hepatoblast marker gene, a myoblast marker gene, a preadipocyte marker gene, a chondrocyte marker gene, a synovial cell marker gene, a tendon cell marker gene, a ligament cell marker gene, a meniscus cell marker gene, an adipose cell marker gene, a dendritic cell marker gene, a natural killer cell marker gene, a muscle cell marker gene, an erythroid-megakaryocytic cell marker gene, an eosinophil marker gene, an islet beta cell marker gene, a neuron marker gene, a cardiomyocyte marker gene, a blood cell marker gene, an exocrine progenitor marker gene, a ductal cell marker gene, an acinar cell marker gene, an alpha cell marker gene, a beta cell marker gene, a delta cell marker gene, a cholangiocyte marker gene, a brown adipocyte marker gene, a cardiac muscle cell marker gene, a PP cell marker gene, an epidermal keratinocyte marker gene, an epithelial cell marker gene, a germ cell marker gene, a skeletal joint synovium cell marker gene, a periosteum cell marker gene, a bone cell marker gene, a perichondrium cell marker gene, a pericardium cell marker gene, a meningeal cell marker gene, a keratinocyte precursor cell marker gene, a keratinocyte stem cell marker gene, a pericyte marker gene, a glial cell marker gene, an ependymal cell marker gene, a serosal cell marker gene, a heart cell marker gene, a brain cell marker gene, a spinal cord cell marker gene, a lung cell marker gene, a pancreas cell marker gene, a bladder cell marker gene, a bone marrow cell marker gene, a spleen cell marker gene, an intestine cell marker gene, a stomach cell marker gene, an islet cell marker gene, a pancreatic islet cell marker gene, an immune cell marker gene, a B cell marker gene, a T cell marker gene, a natural killer (NK) cell marker gene, a natural killer T (NKT) cell marker gene, a macrophage marker gene, an endothelial cell marker gene, a muscle cell marker gene, a smooth muscle cell marker gene, a skeletal muscle cell marker gene, a dopaminergic neuron marker gene, a retinal pigmented epithelium cell marker gene, an optic cell marker gene, a hepatocyte marker gene, a thyroid cell marker gene, a skin cell marker gene, a glial progenitor cell marker gene, a neural cell marker gene, a stem cell marker gene, an endothelial stem cell marker gene, an adipose stem cell marker gene, an adipose progenitor cell marker gene, a lung stem cell marker gene, a lung progenitor cell marker gene, a neural progenitor cell marker gene, a T cell marker gene, a CAR-T cell marker gene, a CD14+ cell marker gene, a dendritic cell marker gene, a PBMC cell marker gene, an exocrine secretory epithelial cell marker gene, a thyroid epithelial cell marker gene, a keratinizing epithelial cell marker gene, a gall bladder epithelial cell marker gene, a surface epithelial cell marker gene, a kidney cell marker gene, a primary T cell marker gene, a CD3+ T cell marker gene, a CD4+T cell marker gene, a CD8+ T cell marker gene, a naive T cell marker gene, a regulatory T (Treg) cell marker gene, a non-regulatory T cell marker gene, a Th1 cell marker gene, a Th2 cell marker gene, a Th9 cell marker gene, a Th17 cell marker gene, a T-follicular helper (Tfh) cell marker gene, a cytotoxic T lymphocyte (CTL) marker gene, an effector T (Teff) cell marker gene, a central memory T (Tcm) cell marker gene, an effector memory T (Tem) cell marker gene, a TEMRA cell marker gene, a tissue-resident memory (Trm) cell marker gene, a virtual memory T cell marker gene, an innate memory T cell marker gene, a memory stem cell (Tsc) marker gene, a 76 T cell marker gene, a helper T cell marker gene, a central memory T cell marker gene, an effector memory T cell marker gene, an effector memory RA T cell marker gene, a tissue infiltrating lymphocyte marker gene, a capillary endothelial cell marker gene, a vascular endothelial cell marker gene, an aortic endothelial cell marker gene, an arterial endothelial cell marker gene, a venous endothelial cell marker gene, a renal endothelial cell marker gene, a brain endothelial cell marker gene, a liver endothelial cell marker gene, a gamma cell marker gene, an epsilon cell marker gene, hematopoietic progenitor cell marker gene, a nodal cardiomyocyte marker gene, a conducting cardiomyocyte marker gene, a working cardiomyocyte marker gene, a cardiomyocyte precursor cell marker gene, a cardiomyocyte progenitor cell marker gene, a cardiac stem cell marker gene, an atrial cardiac stem cell marker gene, a ventricular cardiac stem cell marker gene, an epicardial cell marker gene, a vascular endothelial cell marker gene, an endocardial endothelial cell marker gene, a cardiac valve interstitial cell marker gene, a cardiac pacemaker cell marker gene, a neuroectodermal cell marker gene, a neuronal cell marker gene, a neuroendocrine cell marker gene, a cholinergic cell marker gene, a serotonergic (5-HT) cell marker gene, a glutamatergic cell marker gene, a GABAergic cell marker gene, an adrenergic cell marker gene, a noradrenergic cell marker gene, a sympathetic neuronal cell marker gene, a parasympathetic neuronal cell marker gene, a sympathetic peripheral neuronal cell marker gene, an astrocyte marker gene, an oligodendrocyte marker gene, an ependymal cell marker gene, a radial glia cell marker gene, a Schwann cell marker gene, a cerebral endothelial cells (ECs) marker gene, a neuronal stem cell marker gene, a neuronal progenitor cell marker gene, a white blood cell marker gene, a red blood cell marker gene, a platelet cell marker gene, a tumor cell marker gene, an enterochromaffin cell marker gene, a mesenchymal fibroblast marker gene, an osteoblast marker gene, a stromal cell marker gene, and any combination thereof. 40a. The cell of Embodiment 40, wherein the off-target cell marker gene is selected from the group consisting of a pluripotency cell marker gene, a tumorigenic cell marker gene, a ductal marker gene, an enterochromaffin marker gene, a neural marker gene, acinar marker gene, intestinal marker gene, endothelial marker gene, mesenchymal fibroblast marker gene, muscle marker gene, osteoblast marker gene, and stromal marker gene.

Embodiment 41. The cell of any one of Embodiments 27-40, wherein the off-target cell marker gene is identified using flow cytometry and/or single-cell RNA-sequencing.

Embodiment 42. The cell of any one of Embodiments 27-41, wherein the off-target cell marker gene is selected from the group consisting of ANXA1, KRT19, CTSC, DSC2, ARHGAP29, KRT18, KRT8, CD9, PLK2, and KRT17.

Embodiment 42a. The cell of any one of Embodiments 27-42, wherein the off-target cell marker gene is ANXA1.

Embodiment 42b. The cell of any one of Embodiments 27-42, wherein the off-target cell marker gene is KRT19.

Embodiment 42c. The cell of any one of Embodiments 27-42, wherein the off-target cell marker gene is CTSC.

Embodiment 42d. The cell of any one of Embodiments 27-42, wherein the off-target cell marker gene is DSC2.

Embodiment 42e. The cell of any one of Embodiments 27-42, wherein the off-target cell marker gene is ARHGAP29.

Embodiment 42f. The cell of any one of Embodiments 27-42, wherein the off-target cell marker gene is KRT18.

Embodiment 42g. The cell of any one of Embodiments 27-42, wherein the off-target cell marker gene is KRT8.

Embodiment 42h. The cell of any one of Embodiments 27-42, wherein the off-target cell marker gene is CD9.

Embodiment 42i. The cell of any one of Embodiments 27-42, wherein the off-target cell marker gene is PLK2.

Embodiment 42j. The cell of any one of Embodiments 27-42, wherein the off-target cell marker gene is KRT17.

Embodiment 43. The cell of any one of Embodiments 27-42j, wherein the nucleic acid encoding the selection agent or the detection agent is integrated in a 5′ UTR or a 3′ UTR of the off-target cell marker gene.

Embodiment 43a. The cell of Embodiment 43, wherein the nucleic acid encoding the selection agent or the detection agent is integrated in a 5′ UTR or a 3′ UTR of ANXA1.

Embodiment 43b. The cell of Embodiment 43, wherein the nucleic acid encoding the selection agent or the detection agent is integrated in a 5′ UTR or a 3′ UTR of KRT19.

Embodiment 43c. The cell of Embodiment 43, wherein the nucleic acid encoding the selection agent or the detection agent is integrated in a 5′ UTR or a 3′ UTR of CTSC.

Embodiment 43d. The cell of Embodiment 43, wherein the nucleic acid encoding the selection agent or the detection agent is integrated in a 5′ UTR or a 3′ UTR of DSC2.

Embodiment 43e. The cell of Embodiment 43, wherein the nucleic acid encoding the selection agent or the detection agent is integrated in a 5′ UTR or a 3′ UTR of ARHGAP29.

Embodiment 43f. The cell of Embodiment 43, wherein the nucleic acid encoding the selection agent or the detection agent is integrated in a 5′ UTR or a 3′ UTR of KRT18.

Embodiment 43g. The cell of Embodiment 43, wherein the nucleic acid encoding the selection agent or the detection agent is integrated in a 5′ UTR or a 3′ UTR of KRT8.

Embodiment 43h. The cell of Embodiment 43, wherein the nucleic acid encoding the selection agent or the detection agent is integrated in a 5′ UTR or a 3′ UTR of CD9.

Embodiment 43i. The cell of Embodiment 43, wherein the nucleic acid encoding the selection agent or the detection agent is integrated in a 5′ UTR or a 3′ UTR of PLK2.

Embodiment 43j. The cell of Embodiment 43, wherein the nucleic acid encoding the selection agent or the detection agent is integrated in a 5′ UTR or a 3′ UTR of KRT17.

Embodiment 44. The cell of any one of Embodiments 27-43j, wherein the nucleic acid encoding the selection agent or the detection agent is integrated in an exon or an intron of the off-target cell marker gene.

Embodiment 44a. The cell of Embodiment 44, wherein the nucleic acid encoding the selection agent or the detection agent is integrated in an exon of ANXA1, optionally wherein the exon is exon 1, 2, 3, 4, 5, 6, 7, 8, 9, 10, 11, 12, or 13.

Embodiment 44b. The cell of Embodiment 44, wherein the nucleic acid encoding the selection agent or the detection agent is integrated in an intron of ANXA1, optionally wherein the intron is intron 1, 2, 3, 4, 5, 6, 7, 8, 9, 10, 11, or 12.

Embodiment 44c. The cell of Embodiment 44, wherein the nucleic acid encoding the selection agent or the detection agent is integrated in an exon of KRT19, optionally wherein the exon is exon 1, 2, 3, 4, 5, or 6.

Embodiment 44d. The cell of Embodiment 44, wherein the nucleic acid encoding the selection agent or the detection agent is integrated in an intron of KRT19, optionally wherein the intron is intron 1, 2, 3, 4, or 5.

Embodiment 44e. The cell of Embodiment 44, wherein the nucleic acid encoding the selection agent or the detection agent is integrated in an exon of CTSC, optionally wherein the exon is exon 1, 2, 3, 4, 5, 6, or 7.

Embodiment 44f. The cell of Embodiment 44, wherein the nucleic acid encoding the selection agent or the detection agent is integrated in an intron of CTSC, optionally wherein the intron is intron 1, 2, 3, 4, 5, or 6.

Embodiment 44g. The cell of Embodiment 44, wherein the nucleic acid encoding the selection agent or the detection agent is integrated in an exon of DSC2, optionally wherein the exon is exon 1, 2, 3, 4, 5, 6, 7, 8, 9, 10, 11, 12, 13, 14, 15, or 16.

Embodiment 44h. The cell of Embodiment 44, wherein the nucleic acid encoding the selection agent or the detection agent is integrated in an intron of DSC2, optionally wherein the intron is intron 1, 2, 3, 4, 5, 6, 7, 8, 9, 10, 11, 12, 13, 14, or 15.

Embodiment 44i. The cell of Embodiment 44, wherein the nucleic acid encoding the selection agent or the detection agent is integrated in an exon of ARHGAP29, optionally wherein the exon is exon 1, 2, 3, 4, 5, 6, 7, 8, 9, 10, 11, 12, 13, 14, 15, 16, 17, 18, 19, 20, 21, 22, or 23.

Embodiment 44j. The cell of Embodiment 44, wherein the nucleic acid encoding the selection agent or the detection agent is integrated in an intron of ARHGAP29, optionally wherein the intron is intron 1, 2, 3, 4, 5, 6, 7, 8, 9, 10, 11, 12, 13, 14, 15, 16, 17, 18, 19, 20, 21, or 22.

Embodiment 44k. The cell of Embodiment 44, wherein the nucleic acid encoding the selection agent or the detection agent is integrated in an exon of KRT18, optionally wherein the exon is exon 1, 2, 3, 4, 5, 6, or 7.

Embodiment 441. The cell of Embodiment 44, wherein the nucleic acid encoding the selection agent or the detection agent is integrated in an intron of KRT18, optionally wherein the intron is intron 1, 2, 3, 4, 5, or 6.

Embodiment 44m. The cell of Embodiment 44, wherein the nucleic acid encoding the selection agent or the detection agent is integrated in an exon of KRT8, optionally wherein the exon is exon 1, 2, 3, 4, 5, 6, 7, or 8.

Embodiment 44n. The cell of Embodiment 44, wherein the nucleic acid encoding the selection agent or the detection agent is integrated in an intron of KRT8, optionally wherein the intron is intron 1, 2, 3, 4, 5, 6, or 7.

Embodiment 44o. The cell of Embodiment 44, wherein the nucleic acid encoding the selection agent or the detection agent is integrated in an exon of CD9, optionally wherein the exon is exon 1, 2, 3, 4, 5, 6, 7, or 8.

Embodiment 44p. The cell of Embodiment 44, wherein the nucleic acid encoding the selection agent or the detection agent is integrated in an intron of CD9, optionally wherein the intron is intron 1, 2, 3, 4, 5, 6, or 7.

Embodiment 44q. The cell of Embodiment 44, wherein the nucleic acid encoding the selection agent or the detection agent is integrated in an exon of PLK2, optionally wherein the exon is exon 1, 2, 3, 4, 5, 6, 7, 8, 9, 10, 11, 12, 13, or 14.

Embodiment 44r. The cell of Embodiment 44, wherein the nucleic acid encoding the selection agent or the detection agent is integrated in an intron of PLK2, optionally wherein the intron is intron 1, 2, 3, 4, 5, 6, 7, 8, 9, 10, 11, 12, or 13.

Embodiment 44s. The cell of Embodiment 44, wherein the nucleic acid encoding the selection agent or the detection agent is integrated in an exon of KRT17, optionally wherein the exon is exon 1, 2, 3, 4, 5, 6, 7, or 8.

Embodiment 44t. The cell of Embodiment 44, wherein the nucleic acid encoding the selection agent or the detection agent is integrated in an intron of KRT17, optionally wherein the intron is intron 1, 2, 3, 4, 5, 6, or 7.

Embodiment 45. The cell of any one of Embodiments 27-44t, wherein the selection agent or the detection agent is expressed in a cell with a high level of expression of an off-target cell marker gene.

Embodiment 46. The cell of any one of Embodiments 27-45, wherein the selection agent or the detection agent is expressed in a cell with greater than about 3 transcripts per million (TPM), about 4 TPM, about 5 TPM, about 6 TPM, about 7 TPM, about 8 TPM, about 9 TPM, about 10 TPM, about 15 TPM, about 20 TPM, about 25 TPM, about 30 TPM, about 35 TPM, about 40 TPM, about 45 TPM, or about 50 TPM.

Embodiment 47. The cell of any one of Embodiments 27-46, wherein the selection agent or the detection agent is expressed in an off-target cell with greater than about 3 transcripts per million (TPM), about 4 TPM, about 5 TPM, about 6 TPM, about 7 TPM, about 8 TPM, about 9 TPM, about 10 TPM, about 15 TPM, about 20 TPM, about 25 TPM, about 30 TPM, about 35 TPM, about 40 TPM, about 45 TPM, or about 50 TPM.

Embodiment 48. The cell of any one of Embodiments 27, 28, 30-33, and 35-47, wherein the selection agent is expressed in a cell such that the selection agent enables selection of the cell.

Embodiment 49. The cell of any one of Embodiments 27-29, 32, and 34-47, wherein the detection agent is expressed in a cell such that the detection agent enables detection of the cell.

Embodiment 50. The cell of any one of Embodiments 27-49, wherein the selection agent or the detection agent is expressed in a pluripotent cell.

Embodiment 51. The cell of any one of Embodiments 27-50, wherein the selection agent or the detection agent is expressed in a tumorigenic cell.

Embodiment 52. The cell of any one of Embodiments 27-51, wherein the selection agent or the detection agent is expressed in a ductal cell.

Embodiment 53. The cell of any one of Embodiments 27-52, wherein the selection agent or the detection agent is expressed in an enterochromaffin cell.

Embodiment 54. The cell of any one of Embodiments 27-53, wherein the selection agent or the detection agent is expressed in a neural cell.

Embodiment 55. The cell of any one of Embodiments 27-54, wherein the selection agent or the detection agent is expressed in partially differentiated cells.

Embodiment 56. The cell of any one of Embodiments 27-55, wherein the selection agent or the detection agent is not expressed in differentiated cells.

Embodiment 57. The cell of any one of Embodiments 27-56, wherein the selection agent or the detection agent is not expressed in a cell with a low level of expression or no expression of an off-target cell marker gene.

Embodiment 58. The cell of any one of Embodiments 27-57, wherein the selection agent or the detection agent is not expressed in a therapeutic cell.

Embodiment 59. The cell of any one of Embodiments 27-58, wherein the off-target cell marker gene is not a beta cell marker gene, a T cell marker gene, a neuronal cell marker gene, a glial cell marker gene, a cardiac cell marker gene, a retinal pigment epithelium (RPE) cell marker gene, a hematopoietic progenitor cell marker gene, a natural killer cell marker gene, an endothelial cell marker gene, or a lung cell marker gene.

Embodiment 60. The cell of any one of Embodiments 1-59, wherein the cell further comprises a genome editing complex.

Embodiment 60a. The cell of Embodiment 60, wherein the genome editing complex is encoded by one or more transgenes.

Embodiment 60b. The cell of Embodiment 60 or 60a, wherein the genome editing complex comprises a genome targeting entity and a genome modifying entity.

Embodiment 60c. The cell of Embodiment 60b, wherein the genome targeting entity is a nucleic acid-guided targeting entity.

Embodiment 60d. The cell of any one of Embodiments 60-60c, wherein the genome targeting entity is selected from the group consisting of: a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising a gRNA and a Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), and a Type II or Type V Cas protein, or functional portions thereof.

Embodiment 60e. The cell of any one of Embodiments 60-60d, wherein the genome targeting entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, and dCas13b, or functional portions thereof.

Embodiment 60f. The cell of any one of Embodiments 60-60b, wherein the genome modifying entity cleaves, deaminates, nicks, polymerizes, interrogates, integrates, cuts, unwinds, breaks, alters, methylates, demethylates, or otherwise destabilizes the target locus.

Embodiment 60g. The cell of any one of Embodiments 60-60b and 60f, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.

Embodiment 60h. The cell of any one of Embodiments 60-60b, 60f, and 60g, wherein the genome modifying entity is selected from the group consisting of: a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, an endonuclease-deficient-Cas protein, an enzymatically inactive Cas protein, a CRISPR-associated transposase (CAST), a Type II or Type V Cas protein, base editing, prime editing, and a Programmable Addition via Site-specific Targeting Elements (PASTE), or functional portions thereof.

Embodiment 60i. The cell of any one of Embodiments 60-60b and 60f-60h, wherein the genome modifying entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor, a target-primed reverse transcription (TPRT) editor, APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, and a transcriptional repressor, or functional portions thereof.

Embodiment 60j. The cell of any one of Embodiments 60-60i, wherein the genome targeting entity and the genome modifying entity are different domains of a single polypeptide.

Embodiment 60k. The cell of any one of Embodiments 60-60j, wherein the genome targeting entity and genome modifying entity are two different polypeptides that are operably linked together.

Embodiment 601. The cell of any one of Embodiments 60-60j, wherein the genome targeting entity and genome modifying entity are two different polypeptides that are not linked together.

Embodiment 60m. The cell of any one of Embodiments 60-601, wherein the genome editing complex comprises a guide nucleic acid having a targeting domain that is complementary to at least one target locus, optionally wherein the guide nucleic acid is a guide RNA (gRNA).

Embodiment 60n. The cell of any one of Embodiments 60-60m, wherein the genome editing complex is an RNA-guided nuclease.

Embodiment 60o. The cell of Embodiment 60n, wherein the RNA-guided nuclease comprises a Cas nuclease and a guide RNA (CRISPR-Cas combination).

Embodiment 60p. The cell of Embodiment 60o, wherein the CRISPR-Cas combination is a ribonucleoprotein (RNP) complex comprising the gRNA and the Cas nuclease.

Embodiment 60q. The cell of Embodiment 60o or 60p, wherein the Cas nuclease is a Type II or Type V Cas protein.

Embodiment 60r. The cell of any one of Embodiments 60o-60q, wherein the Cas nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7.

Embodiment 61. A composition comprising the cell of any one of the preceding Embodiments.

Embodiment 62. The composition of Embodiment 61, wherein the cell is an allogeneic cell. 62a. The composition of Embodiment 62, wherein the allogeneic cell is derived from one or more donors.

Embodiment 63. The composition of Embodiment 61, wherein the cell is an autologous cell.

Embodiment 64. A method of eliminating proliferating cells comprising providing a population of cells with an inducer, wherein the population of cells express a nucleic acid encoding a selection agent integrated at an endogenous proliferation gene locus and operably linked to the promoter of the endogenous proliferation gene, and wherein providing the inducer causes elimination of proliferating cells.

Embodiment 65. The method of Embodiment 64, wherein the providing is administering the inducer to an individual harboring the population of cells.

Embodiment 66. The method of Embodiment 64 or 65, wherein the method further comprises administering the population of cells to a subject either before or after the inducer is provided to the subject.

Embodiment 67. The method of Embodiment 66, wherein the population of cells is incubated with the inducer prior to administration to the subject.

Embodiment 68. The method of Embodiment 66, wherein the population of cells are administered to a subject before the inducer is provided to the subject.

Embodiment 69. The method of Embodiment 64, wherein the providing comprises incubating the population of cells in vitro.

Embodiment 70. The method of any one of Embodiments 64-69, wherein the endogenous proliferation gene locus is selected from the group consisting of AURKB, CDC20, RRM2, CDK1, BIRC5, TOP2A, PTTG1, CCNB1, TPX2, KIF11, SPC25, CENPK, SMC4, TYMS, H2AZ1, TMSB15A, CENPF, and MKI67.

Embodiment 70a. The method of any one of Embodiments 64-70, wherein the endogenous proliferation gene locus is selected from the group consisting of AURKB, RRM2, TOP2A, PTTG1, CCNB1, SPC25, CENPK, SMC4, TYMS, H2AZ1, TMSB15A, CENPF, and MKI67.

Embodiment 70b. The method of any one of Embodiments 64-70a, wherein the endogenous proliferation gene is AURKB.

Embodiment 70c. The method of any one of Embodiments 64-70a, wherein the endogenous proliferation gene is RRM2.

Embodiment 70d. The method of any one of Embodiments 64-70a, wherein the endogenous proliferation gene is TOP2A.

Embodiment 70e. The method of any one of Embodiments 64-70a, wherein the endogenous proliferation gene is PTTG1.

Embodiment 70f. The method of any one of Embodiments 64-70a, wherein the endogenous proliferation gene is CCNB1.

Embodiment 70g. The method of any one of Embodiments 64-70a, wherein the endogenous proliferation gene is SPC25.

Embodiment 70h. The method of any one of Embodiments 64-70a, wherein the endogenous proliferation gene is CENPK.

Embodiment 70i. The method of any one of Embodiments 64-70a, wherein the endogenous proliferation gene is SMC4.

Embodiment 70j. The method of any one of Embodiments 64-70a, wherein the endogenous proliferation gene is TYMS.

Embodiment 70k. The method of any one of Embodiments 64-70a, wherein the endogenous proliferation gene is H2AZ1.

Embodiment 701. The method of any one of Embodiments 64-70a, wherein the endogenous proliferation gene is TMSB15A.

Embodiment 70m. The method of any one of Embodiments 64-70a, wherein the endogenous proliferation gene is CENPF.

Embodiment 70n. The method of any one of Embodiments 64-70a, wherein the endogenous proliferation gene is MKI67.

Embodiment 71. The method of any one of Embodiments 64-70n, wherein the method selectively eliminates proliferating cells, cancer cells, pluripotent cells, multipotent stem cells, progenitor cells, de-differentiated cells, undifferentiated cells, and/or partially differentiated cells.

Embodiment 72. The method of any one of Embodiments 64-71, wherein the population of cells comprises proliferating cells and non-proliferating cells.

Embodiment 73. The method of any one of Embodiments 64-72, wherein the differentiated cells and/or non-proliferating cells are not eliminated.

Embodiment 74. A method of eliminating a specific cell type comprising providing a population of cells with an inducer, wherein the population of cells express a nucleic acid encoding a kill switch from an off-target cell marker gene locus and operably linked to the promoter of the off-target cell marker gene, and wherein providing the inducer causes elimination of an off-target cell.

Embodiment 75. The method of Embodiment 74, wherein the providing is administering the inducer to an individual harboring the population of cells.

Embodiment 76. The method of Embodiment 74 or 75, wherein the method further comprises administering the population of cells to a subject either before or after the inducer is provided to the subject.

Embodiment 77. The method of Embodiment 76, wherein the population of cells is incubated with the inducer prior to administration to the subject.

Embodiment 78. The method of Embodiment 76, wherein the population of cells are administered to a subject before the inducer is provided to the subject.

Embodiment 79. The method of Embodiment 74, wherein the providing comprises incubating the population of cells in vitro.

Embodiment 80. The method of any one of Embodiments 74-79, wherein the off-target cell marker gene is selected from the group consisting of ANXA1, KRT19, CTSC, DSC2, ARHGAP29, KRT18, KRT8, CD9, PLK2, and KRT17.

Embodiment 80a. The cell of any one of Embodiments 74-80, wherein the off-target cell marker gene is ANXA1.

Embodiment 80b. The method of any one of Embodiments 74-80, wherein the off-target cell marker gene is KRT19.

Embodiment 80c. The method of any one of Embodiments 74-80, wherein the off-target cell marker gene is CTSC.

Embodiment 80d. The method of any one of Embodiments 74-80, wherein the off-target cell marker gene is DSC2.

Embodiment 80e. The method of any one of Embodiments 74-80, wherein the off-target cell marker gene is ARHGAP29.

Embodiment 80f. The method of any one of Embodiments 74-80, wherein the off-target cell marker gene is KRT18.

Embodiment 80g. The method of any one of Embodiments 74-80, wherein the off-target cell marker gene is KRT8.

Embodiment 80h. The method of any one of Embodiments 74-80, wherein the off-target cell marker gene is CD9.

Embodiment 80i. The method of Embodiment any one of Embodiments 74-80, wherein the off-target cell marker gene is PLK2.

Embodiment 80j. The method of Embodiment any one of Embodiments 74-80, wherein the off-target cell marker gene is KRT17.

Embodiment 81. The method of any one of Embodiments 74-80j, wherein the method selectively eliminates cancer cells, pluripotent cells, multipotent stem cells, progenitor cells, de-differentiated cells, undifferentiated cells, partially differentiated cells, and/or off-target cells.

Embodiment 82. The method of any one of Embodiments 74-81, wherein the inducer does not cause elimination of a cell type other than the off-target cell.

Embodiment 83. The method of any one of Embodiments 74-82, wherein on-target cells are not eliminated.

Embodiment 84. The method of any one of Embodiments 74-83, wherein the population of cells comprises on-target cells and off-target cells.

Embodiment 85. The method of any one of Embodiments 74-84, wherein the population of cells comprises therapeutic cells and off-target cells.

Embodiment 86. The method of any one of Embodiments 74-85, wherein therapeutic cells are not eliminated.

Embodiment 87. The method of any one of Embodiments 74-86, wherein the population of cells are stem cell derived cells or primary cells.

Embodiment 88. The method of any one of Embodiments 74-87, wherein the stem cells are pluripotent stem cells, embryonic stem cells, induced pluripotent stem cells, multipotent stem cells, or adult stem cells.

Embodiment 89. The method of any one of Embodiments 74-88, wherein the cells are autologous cells.

Embodiment 90. The method of any one of Embodiments 74-88, wherein the cells are allogeneic cells.

Embodiment 90a. The method of Embodiment 90, wherein the allogeneic cells are derived from one or more donors.

Embodiment 91. The method of any one of Embodiments 64-90a, wherein the inducer is administered if cell proliferation is detected or if information indicating cell proliferation is obtained.

Embodiment 92. The method of any one of Embodiments 64-91, wherein the inducer is administered if expression of proliferation markers is detected or if information indicating expression of proliferation markers is obtained.

Embodiment 93. The method of any one of Embodiments 64-92, wherein the inducer is administered if an excess number of cells is detected or if information indicating an excess number of cells is obtained.

Embodiment 94. The method of any one of Embodiments 64-93, further comprising detecting proliferation using flow cytometry for a proliferation marker.

Embodiment 95. The method of any one of Embodiments 64-94, further comprising detecting proliferation using single-cell RNA-sequencing for expression of proliferation markers.

Embodiment 96. A method of detecting a proliferating cell expressing a detection agent in a population of cells, wherein the population of cells comprises a nucleic acid encoding the detection agent integrated at an endogenous proliferation gene locus and operably linked to the promoter of the endogenous proliferation gene, and wherein expression of the nucleic acid encoding the detection agent is regulated by the promoter of the endogenous proliferation gene and/or regulatory elements of the endogenous proliferation gene.

Embodiment 97. A method of detecting a proliferating cell expressing a detection agent in a population of cells isolated from an individual, wherein the population of cells comprises a nucleic acid encoding the detection agent integrated at an endogenous proliferation gene locus and operably linked to the promoter of the endogenous proliferation gene, and wherein expression of the nucleic acid encoding the detection agent is regulated by the promoter of the endogenous proliferation gene and/or regulatory elements of the endogenous proliferation gene.

Embodiment 98. A method of detecting a proliferating cell expressing a detection agent in a population of cells in an individual, wherein the population of cells comprises a nucleic acid encoding the detection agent integrated at an endogenous proliferation gene locus and operably linked to the promoter of the endogenous proliferation gene, and wherein expression of the nucleic acid encoding the detection agent is regulated by the promoter of the endogenous proliferation gene and/or regulatory elements of the endogenous proliferation gene.

Embodiment 99. A method of detecting an off-target cell expressing a detection agent in a population of cells, wherein the population of cells comprises a nucleic acid encoding the detection agent integrated at an off-target cell marker gene locus and operably linked to the promoter of the off-target cell marker gene, and wherein expression of the nucleic acid encoding the detection agent is regulated by the promoter of the off-target cell marker gene and/or regulatory elements of the off-target cell marker gene.

Embodiment 100. A method of detecting an off-target cell expressing a detection agent in a population of cells isolated from an individual, wherein the population of cells comprises a nucleic acid encoding the detection agent integrated at an off-target cell marker gene locus and operably linked to the promoter of the off-target cell marker gene, and wherein expression of the nucleic acid encoding the detection agent is regulated by the promoter of the off-target cell marker gene and/or regulatory elements of the off-target cell marker gene.

Embodiment 101. A method of detecting an off-target cell expressing a detection agent in a population of cells in an individual, wherein the population of cells comprises a nucleic acid encoding the detection agent integrated at an off-target cell marker gene locus and operably linked to the promoter of the off-target cell marker gene, and wherein expression of the nucleic acid encoding the detection agent is regulated by the promoter of the off-target cell marker gene and/or regulatory elements of the off-target cell marker gene.

Embodiment 102. The method of any one of Embodiments 96-101, comprising detecting the presence or absence of a first barcode and/or a second barcode in the cell or population of cells, wherein the presence of the first barcode indicates the presence of a first transgene and the presence of the second barcode indicates the presence of a second transgene, and selecting the cell or population of cells as being suitable for (a) administration to a subject; (b) manufacturing a drug product; (c) gene editing or further gene editing; (d) viral transduction or further viral transduction; (e) creating a cell bank; (f) differentiation into a cellular intermediate or a fully differentiated drug product; (g) packaging for distribution; and/or (h) cryopreservation or formulation, based on the presence or absence of the first barcode and/or second barcode.

Embodiment 103. The method of Embodiment 102, wherein the therapy is a cell therapy.

Embodiment 104. The method of Embodiment 103, wherein the cell therapy is generated in vivo or ex vivo.

Embodiment 105. The method of Embodiment 104, wherein the cell therapy is generated in vivo in a subject in need thereof to treat a disease in the subject, wherein a vector comprising the first transgene and/or the second transgene and the first barcode and/or the second barcode is administered to the subject.

Embodiment 106. The method of Embodiment 105, wherein the vector is packaged in a fusosome for trafficking to the target cell or target population of cells in vivo within the subject.

Embodiment 107. The method of any one of Embodiments 96-104, further comprising administering to a subject in need thereof an effective dose of a cell, population of cells, or cell therapy to treat a disease in the subject.

Embodiment 108. The method of Embodiment 107, wherein the cell therapy comprises administering one or more doses of engineered immune cells expressing the first transgene and/or the second transgene.

Embodiment 109. The method of any one of Embodiments 103-108, further comprising monitoring a sample obtained from the subject administered the therapy for the presence or absence of the first barcode and/or the second barcode in the cell, population of cells, or therapy, the method comprising: (i) detecting the first barcode and/or the second barcode in the sample obtained from the subject receiving the cell, population of cells, or therapy, wherein detection of the first barcode indicates the presence of the first transgene, and/or detection of the second barcode indicates the presence of the second transgene in the sample; and (ii) determining the percentage, ratio, relative, or absolute number of cells expressing the first transgene and/or the second transgene.

Embodiment 110. The method of Embodiment 109, further comprising determining the percentage, ratio, relative, or absolute number of cells expressing the first transgene and/or the second transgene at a second time point.

Embodiment 111. The method of Embodiment 110, wherein determining the percentage, ratio, relative, or absolute number of cells expressing the first transgene and/or the second transgene in a sample at a first time point and a second time point is used to monitor: i) cell therapy persistence; ii) cell therapy efficacy; iii) expansion of the cells expressing the first transgene and/or the second transgene; or iv) changes in a subject's health or disease profile, in a subject receiving treatment.

Embodiment 112. The method of any one of Embodiments 107-111, further comprising one or more additional administrations of the cell, population of cells, or therapy to the subject comprising: i) the same or different dose as the initial dose of the cell, population of cells, or therapy administered to the subject; and/or ii) a cell, population of cells, or therapy comprising the same or different transgene encoded by a vector as the initial cell, population of cells, or therapy administered to the subject.

Embodiment 113. The method of Embodiment 112, comprising obtaining a sample from the subject who was administered the therapy, detecting the presence or absence of a first barcode and/or a second barcode in the sample, wherein the presence of the first barcode indicates the presence of a first transgene, and the presence of the second barcode indicates the presence of a second transgene, and determining the percentage, ratio, relative, or absolute number of cells expressing the first transgene and/or the second transgene based on the presence or absence of the first barcode and/or second barcode.

Embodiment 114. The method of any one of Embodiments 102-113, wherein the presence of the barcode indicates the presence of the detection agent.

Embodiment 115. The method any one of Embodiments 102-113, wherein the presence of the barcode indicates the presence of the selection agent.

Embodiment 116. The method of any one of Embodiments 96-115, further comprising removing or eliminating the proliferating cell or the off-target cell if expression of the detection agent is identified.

Embodiment 116a. The method of any one of Embodiments 64-116, further comprising treating the population of cells with an agent that recognizes the detection agent, wherein treating the population of cells with the agent that recognizes the detection agent eliminates the proliferating cell or the off-target cell or targets the proliferating cell or the off-target cell for elimination.

Embodiment 116b. The method of any one of Embodiments 64-116, further comprising surgically removing the proliferating cell or the off-target cell if expression of the detection agent is identified.

Embodiment 116c. The method of any one of Embodiments 64-116, further comprising purifying the population of cells to select for cells not expressing the detection agent prior to administration to a patient if expression of the detection agent is identified.

Embodiment 116d. The method of any one of Embodiments 64-116, further comprising modifying a treatment when the proliferating cell or the off-target cell expressing the detection agent is identified.

Embodiment 116e. The method of any one of Embodiments 64-116, further comprising administering an additional therapy when the proliferating cell or the off-target cell expressing the detection agent is identified.

Embodiment 116f. The method of any one of Embodiments 64-116, further comprising administering cells that are not expressing the detection agent to a subject.

Embodiment 116g. The method of any one of Embodiments 64-116, wherein a treatment regimen is not modified when there are no cells expressing the detection agent identified.

Embodiment 117. A method of selecting a cell population that is suitable for cell therapy, comprising detecting a proliferating cell expressing a detection agent in a population of cells, wherein the population of cells comprises a nucleic acid encoding the detection agent integrated at an endogenous proliferation gene locus and operably linked to the promoter of the endogenous proliferation gene.

Embodiment 118. The method of Embodiment 117, wherein the selected cell population comprises less than about 50%, about 45%, about 40%, about 35%, about 30%, about 25%, about 20%, about 15%, about 10%, about 9%, about 8%, about 7%, about 6%, about 5%, about 4%, about 3%, about 2%, or about 1% of cells expressing the detection agent.

Embodiment 119. The method of Embodiment 117 or 118, wherein the selected cell population comprises less than about 30-50%, about 20-40%, about 10-30%, about 5-20%, about 1-20%, about 1-15%, about 1-10%, about 1-9%, about 1-8%, about 1-7%, about 1-6%, about 1-5%, about 1-4%, about 1-3%, or about 1-2% of the total cell population.

Embodiment 120. The method of any one of Embodiments 117-119, wherein the selected cell population comprises less than about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% of the total cell population.

Embodiment 121. A method of selecting a cell population that is suitable for cell therapy, comprising detecting an off-target cell expressing a detection agent in a population of cells, wherein the population of cells comprises a nucleic acid encoding the detection agent integrated at an off-target cell marker gene locus and operably linked to the promoter of the off-target cell marker gene.

Embodiment 122. The method of Embodiment 121, wherein the selected cell population comprises less than about 50%, about 45%, about 40%, about 35%, about 30%, about 25%, about 20%, about 15%, about 10%, about 9%, about 8%, about 7%, about 6%, about 5%, about 4%, about 3%, about 2%, or about 1% of cells expressing the detection agent.

Embodiment 123. The method of Embodiment 121 or 122, wherein the selected cell population comprises less than about 30-50%, 20-40%, 10-30%, 5-20%, 1-20%, 1-15%, 1-10%, about 1-9%, about 1-8%, about 1-7%, about 1-6%, about 1-5%, about 1-4%, about 1-3%, or about 1-2% of the total cell population.

Embodiment 124. The method of any one of Embodiments 121-123, wherein the selected cell population comprises less than about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% of the total cell population.

Embodiment 125. A method of making a cell comprising a selection agent or a detection agent integrated at an endogenous proliferation gene locus and operably linked to the promoter of the endogenous proliferation gene, the method comprising introducing a nucleic acid encoding a selection agent or a detection agent into the cell, wherein the nucleic acid encoding the selection agent or the detection agent is integrated in the genome of the cell at the endogenous proliferation gene locus.

Embodiment 126. A method of making a cell comprising a selection agent or a detection agent integrated at an off-target cell marker gene locus and operably linked to the promoter of the off-target marker gene, the method comprising introducing a nucleic acid encoding a selection agent or a detection agent into the cell, wherein the nucleic acid encoding the selection agent or the detection agent is integrated in the genome of the cell at the off-target cell marker gene locus.

Embodiment 127. The method of Embodiment 125 or 126, wherein the integration of a nucleic acid comprises a gene knock-in.

Embodiment 127a. The method of Embodiment 125 or 126, wherein the integration of a nucleic acid comprises a gene knock-out.

Embodiment 128. The method of any one of Embodiments 125-127a, wherein the integration comprises a targeted integration method.

Embodiment 129. The method of any one of Embodiments 125-128, wherein the method further comprises a genome editing complex.

Embodiment 129a. The method of Embodiment 129, wherein the genome editing complex is encoded by one or more transgenes.

Embodiment 129b. The method of Embodiment 129 or 129a, wherein the genome editing complex comprises a genome targeting entity and a genome modifying entity.

Embodiment 129c. The method of Embodiment 129b, wherein the genome targeting entity is a nucleic acid-guided targeting entity.

Embodiment 129d. The method of Embodiment 129-129c, wherein the genome targeting entity is selected from the group consisting of: a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising a gRNA and a Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), and a Type II or Type V Cas protein, or functional portions thereof.

Embodiment 129e. The method of any one of Embodiments 129-129d, wherein the genome targeting entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, and dCas13b, or functional portions thereof.

Embodiment 129f. The method of Embodiment 129-129b, wherein the genome modifying entity cleaves, deaminates, nicks, polymerizes, interrogates, integrates, cuts, unwinds, breaks, alters, methylates, demethylates, or otherwise destabilizes the target locus.

Embodiment 129g. The method of Embodiment 129-129b and 129f, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.

Embodiment 129h. The method of any one of Embodiments 129-129b, 129f, and 129g, wherein the genome modifying entity is selected from the group consisting of: a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, an endonuclease-deficient-Cas protein, an enzymatically inactive Cas protein, a CRISPR-associated transposase (CAST), a Type II or Type V Cas protein, base editing, prime editing, and a Programmable Addition via Site-specific Targeting Elements (PASTE), or functional portions thereof.

Embodiment 129i. The method of any one of Embodiments 129-129b and 129f-129h, wherein the genome modifying entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor, a target-primed reverse transcription (TPRT) editor, APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, and a transcriptional repressor, or functional portions thereof.

Embodiment 129j. The method of any one of Embodiments 129-129i, wherein the genome targeting entity and the genome modifying entity are different domains of a single polypeptide.

Embodiment 129k. The method of any one of Embodiments 129-129j, wherein the genome targeting entity and genome modifying entity are two different polypeptides that are operably linked together.

Embodiment 129l. The method of any one of Embodiments 129-129j, wherein the genome targeting entity and genome modifying entity are two different polypeptides that are not linked together.

Embodiment 129m. The method of any one of Embodiments 129-129l, wherein the genome editing complex comprises a guide nucleic acid having a targeting domain that is complementary to at least one target locus, optionally wherein the guide nucleic acid is a guide RNA (gRNA).

Embodiment 129n. The method of any one of Embodiments 129-129m, wherein the genome editing complex is an RNA-guided nuclease.

Embodiment 129o. The method of Embodiment 129n, wherein the RNA-guided nuclease comprises a Cas nuclease and a guide RNA (CRISPR-Cas combination).

Embodiment 129p. The method of Embodiment 129o, wherein the CRISPR-Cas combination is a ribonucleoprotein (RNP) complex comprising the gRNA and the Cas nuclease.

Embodiment 129q. The method of Embodiment 129o or 129p, wherein the Cas nuclease is a Type II or Type V Cas protein.

Embodiment 129r. The method of any one of Embodiments 129o-129q, wherein the Cas nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7.

Embodiment 130. The method of any one of Embodiments 128-129r, wherein the targeted integration method is a Cas-directed homology-directed repair (HDR).

Embodiment 131. A method of treating a patient in need thereof comprising, (c) administering a population of cells to the patient, wherein the population of cells comprise a nucleic acid encoding a selection agent integrated at an endogenous proliferation gene locus and operably linked to the promoter of the endogenous proliferation gene, and (d) activating the selection agent to eliminate one or more cells in the population of cells.

Embodiment 132. A method of treating a patient in need thereof comprising, (a) administering a population of cells to the patient, wherein the population of cells comprise a nucleic acid encoding a selection agent integrated at an off-target cell marker gene locus and operably linked to the promoter of the off-target cell marker gene, and (b) activating the selection agent to eliminate one or more cells in the population of cells.

Embodiment 132a. The method of Embodiment 131 or 132, wherein the step of activating the selection agent takes place before the population of cells are administered to the patient and/or after the population of cells are administered to the patient.

Embodiment 132b. The method of Embodiment 132a, wherein the step of activating the selection agent takes place before the population of cells are administered to the patient.

Embodiment 132c. The method of Embodiment 132a, wherein the step of activating the selection agent takes place after the population of cells are administered to the patient.

Embodiment 132d. The method of Embodiment 132a, wherein the step of activating the selection agent takes place before the population of cells are administered to the patient and after the population of cells are administered to the patient.

Embodiment 132e. A method of treating a patient in need thereof comprising activating a selection agent to eliminate one or more cells in a population of cells, wherein the patient was previously administered the population of cells, wherein the population of cells comprise a nucleic acid encoding a selection agent integrated at an endogenous proliferation gene locus and operably linked to the promoter of the endogenous proliferation gene.

Embodiment 132f. A method of treating a patient in need thereof comprising activating a selection agent to eliminate one or more cells in a population of cells, wherein the patient was previously administered the population of cells, wherein the population of cells comprise a nucleic acid encoding a selection agent integrated at an off-target cell marker gene locus and operably linked to the promoter of the off-target cell marker gene.

Embodiment 133. A method of treating a disease in an individual, comprising administering a cell therapy to treat the disease, wherein the cell therapy comprises a nucleic acid encoding a selection agent or a detection agent integrated at an endogenous proliferation gene locus and operably linked to the promoter of the endogenous proliferation gene.

Embodiment 134. A method of treating a disease in an individual, comprising administering a cell therapy to treat the disease, wherein the cell therapy comprises a nucleic acid encoding a selection agent or a detection agent integrated at an off-target cell marker gene locus and operably linked to the promoter of the off-target cell marker gene.

Embodiment 135. The method of Embodiment 133 or 134, wherein the disease is a cellular deficiency.

Embodiment 136. The method of Embodiment 135, wherein the disease is associated with diabetes or is diabetes.

Embodiment 137. The method of Embodiment 136, wherein the diabetes is Type I diabetes.

Embodiment 138. The method of Embodiment 133 or 134, wherein the disease is a cancer, a neurological disease, or an autoimmune disease.

Embodiment 139. The method of Embodiment 138, wherein the cancer is selected from the group consisting of ovarian cancer, gastric adenocarcinoma, pancreatic adenocarcinoma, glioblastoma, hepatocellular carcinoma, B-cell chronic lymphocytic leukemia (B-CLL), juvenile chronic myelogenous leukemia (CML), juvenile myelomonocytic leukemia (JMML), Acute Lymphoblastic Leukemia (ALL), Acute Myeloid Leukemia (AML), Adrenocortical Carcinoma, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Atypical Teratoid/Rhabdoid Tumor, Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors, Burkitt Lymphoma, Carcinoid Tumor, Carcinoma, Cardiac Tumors, Atypical Teratoid/Rhabdoid Tumor, Medulloblastoma, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Endometrial Cancer, Ependymoma, Esophageal Cancer, Esthesioneuroblastoma, Ewing Sarcoma, Extracranial Germ Cell Tumor, Extragonadal Germ Cell Tumor, Intraocular Melanoma, Retinoblastoma, Fallopian Tube Cancer, Fibrous Histiocytoma of Bone, Osteosarcoma, Gallbladder Cancer, Gastric Cancer, Gastrointestinal Carcinoid Tumor, Gastrointestinal Stromal Tumors (GIST) (Soft Tissue Sarcoma), Germ Cell Tumors, Central Nervous System Germ Cell Tumors, Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Hepatocellular Cancer, Histiocytosis (Langerhans Cell), Hodgkin Lymphoma, Hypopharyngeal Cancer, Islet Cell Tumors, Pancreatic Neuroendocrine Tumors, Kaposi Sarcoma (Soft Tissue Sarcoma), Renal Cell Cancer, Langerhans Cell Histiocytosis, Laryngeal Cancer, Leukemia, Lip and Oral Cavity Cancer, Liver Cancer, Lung Cancer (Non-Small Cell, Small Cell, Pleuropulmonary Blastoma, and Tracheobronchial Tumor), Lung Squamous Cell Carcinoma, Lymphoma, Male Breast Cancer, Malignant Fibrous Histiocytoma of Bone and Osteosarcoma, Merkel Cell Carcinoma, Mesothelioma, Metastatic Cancer, Metastatic Squamous Neck Cancer with Occult Primary, Midline Tract Carcinoma With NUT Gene Changes, Oropharyngeal Cancer, Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Chronic Myeloproliferative Neoplasms, acute B lymphoblastic leukemia (B-ALL), large B cell lymphoma (LBCL), diffuse large B cell lymphoma (DLBCL), high-grade B cell lymphoma (HGBCL), primary mediastinal B cell lymphoma (PMBCL), mantle cell lymphoma (MCL), marginal zone lymphoma (MZL), or small lymphocytic lymphoma (SLL) Follicular Lymphoma, Blastic Plasmacytoid Dendritic Cell Neoplasm (BPDCN), Systemic Mastocytosis, Nasal Cavity and Paranasal Sinus Cancer, Nasopharyngeal Cancer, Neuroblastoma, Non-Hodgkin Lymphoma, Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Primary Peritoneal Cancer, Prostate Cancer, Recurrent Cancer, Rhabdomyosarcoma, Salivary Gland Cancer, Vascular Tumors, Small Intestine Cancer, Soft Tissue Sarcoma, T-Cell Lymphoma, Thymoma and Thymic Carcinoma, Transitional Cell Cancer of the Renal Pelvis and Ureter, Vaginal Cancer, Vulvar Cancer, and Wilms Tumor.

Embodiment 140. The method of Embodiment 138, wherein the autoimmune disease is selected from the group consisting of arthritis, rheumatoid arthritis, acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, juvenile-onset rheumatoid arthritis, osteoarthritis, arthritis chronica progrediente, arthritis deformans, polyarthritis chronica primaria, reactive arthritis, ankylosing spondylitis, inflammatory hyperproliferative skin diseases, psoriasis, plaque psoriasis, gutatte psoriasis, pustular psoriasis, psoriasis of the nails, atopy, atopic diseases, hay fever, Job's syndrome, dermatitis, contact dermatitis, chronic contact dermatitis, exfoliative dermatitis, exfoliative psoriatic dermatitis, allergic dermatitis, allergic contact dermatitis, dermatitis herpetiformis, nummular dermatitis, seborrheic dermatitis, non-specific dermatitis, primary irritant contact dermatitis, atopic dermatitis, x-linked hyper IgM syndrome, allergic intraocular inflammatory diseases, urticaria, chronic allergic urticaria, chronic idiopathic urticaria, chronic autoimmune urticaria, myositis, polymyositis/dermatomyositis, juvenile dermatomyositis, toxic epidermal necrolysis, scleroderma, systemic scleroderma, sclerosis, systemic sclerosis, multiple sclerosis (MS), MS associated with EBV infection, spino-optical MS, primary progressive MS (PPMS), relapsing-remitting MS (RRMS), progressive relapsing MS, secondary progressive MS (SPMS), progressive systemic sclerosis, atherosclerosis, arteriosclerosis, sclerosis disseminata, ataxic sclerosis, neuromyelitis optica spectrum disorder, inflammatory bowel disease (IBD), Crohn's disease, autoimmune-mediated gastrointestinal diseases, colitis, ulcerative colitis, colitis ulcerosa, microscopic colitis, collagenous colitis, colitis polyposa, necrotizing enterocolitis, transmural colitis, autoimmune inflammatory bowel disease, bowel inflammation, pyoderma gangrenosum, erythema nodosum, primary sclerosing cholangitis, respiratory distress syndrome, adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage, meningitis, herpes gestationis, pemphigoid gestationis, pruritis scroti, autoimmune premature ovarian failure, sudden hearing loss due to an autoimmune condition, IgE-mediated diseases, anaphylaxis, allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN) with or without nephrotic syndrome, chronic or acute glomerulonephritis, primary GN, immune-mediated GN, membranous GN (membranous nephropathy), idiopathic membranous GN or idiopathic membranous nephropathy, membrano- or membranous proliferative GN (MPGN), Type I or Type II GN, rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, erythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema, allergic or atopic eczema, asteatotic eczema, dyshidrotic eczema, vesicular palmoplantar eczema, asthma, asthma bronchiale, bronchial asthma, auto-immune asthma, conditions involving infiltration of T cells or chronic inflammatory responses, immune reactions against foreign antigens, immune reactions against fetal A-B-O blood groups during pregnancy, chronic pulmonary inflammatory disease, autoimmune myocarditis, leukocyte adhesion deficiency, lupus, lupus nephritis, lupus cerebritis, pediatric lupus, non-renal lupus, extra-renal lupus, discoid lupus, discoid lupus erythematosus, alopecia lupus, systemic lupus erythematosus (SLE), cutaneous SLE, subacute cutaneous SLE, neonatal lupus syndrome (NLE), lupus erythematosus disseminatus, CNS lupus, anti-neutrophilic cytoplasmic autoantibody (ANCA) associated vasculitis, granulomatous polyangiitis, microscopic polyangiitis, autoimmune blistering skin diseases, anti-NMDA receptor neuropathy, stiff persons disease, anti-NMDA receptor encephalitis, anti-synthetase autoimmune syndromes, rapidly progressive glomerulopathy, Type I diabetes, Type II diabetes, latent autoimmune diabetes in adults, Type 1.5 diabetes, juvenile onset (Type I) diabetes mellitus, pediatric insulin-dependent diabetes mellitus (IDDM), adult onset diabetes mellitus (Type II diabetes), idiopathic diabetes, insipidus, diabetic retinopathy, diabetic nephropathy, diabetic large-artery disorder, immune responses associated with acute or delayed hypersensitivity mediated by cytokines or T-lymphocytes, tuberculosis, sarcoidosis, granulomatosis, lymphomatoid granulomatosis, Wegener's granulomatosis, agranulocytosis, vasculitides, vasculitis, large-vessel vasculitis, polymyalgia rheumatica, giant cell (Takayasu's) arteritis, medium-vessel vasculitis, Kawasaki's disease, polyarteritis nodosa/periarteritis nodosa, microscopic polyarteritis, immunovasculitis, CNS vasculitis, cutaneous vasculitis, hypersensitivity vasculitis, necrotizing vasculitis, systemic necrotizing vasculitis, ANCA-associated vasculitis, Churg-Strauss vasculitis, syndrome (CSS), ANCA-associated small-vessel vasculiti, temporal arteritis, aplastic anemia, autoimmune aplastic anemia, Coombs positive anemia, Diamond Blackfan anemia, hemolytic anemia, immune hemolytic anemia, autoimmune hemolytic anemia (AIHA), pernicious anemia (anemia perniciosa), Addison's disease, pure red cell anemia, aplasia (PRCA), Factor VIII deficiency, hemophilia A, autoimmune neutropenia, pancytopenia, leukopenia, diseases involving leukocyte diapedesis, CNS inflammatory disorders, Alzheimer's disease, Parkinson's disease, multiple organ injury syndrome, multiple organ injury syndrome secondary to septicemia, trauma, or hemorrhage, antigen-antibody complex-mediated diseases, anti-glomerular basement membrane disease, anti-phospholipid antibody syndrome, anti-phospholipid syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid, pemphigoid bullous, skin pemphigoid, pemphigus, pemphigus vulgaris, pemphigus foliaceus, pemphigus mucus-membrane pemphigoid, pemphigus erythematosus, autoimmune polyendocrinopathies, Reiter's disease or syndrome, thermal injury, preeclampsia, an immune complex disorder, immune complex nephritis, antibody-mediated nephritis, polyneuropathies, chronic neuropathy, IgM polyneuropathies, IgM-mediated neuropathy, thrombocytopenia, thrombotic thrombocytopenic purpura (TTP), post-transfusion purpura (PTP), heparin-induced thrombocytopenia, autoimmune or immune-mediated thrombocytopenia, idiopathic thrombocytopenic purpura (ITP), chronic or acute ITP, acquired thrombocytopenic purpura, scleritis, idiopathic cerato-scleritis, episcleritis, autoimmune disease of the testis or ovary, autoimmune orchitis or oophoritis, primary hypothyroidism, hypoparathyroidism, autoimmune endocrine diseases, thyroiditis, autoimmune thyroiditis, Hashimoto's disease, chronic thyroiditis, Hashimoto's thyroiditis, subacute thyroiditis, autoimmune thyroid disease, idiopathic hypothyroidism, Grave's disease, polyglandular syndromes, autoimmune polyglandular syndromes, polyglandular endocrinopathy syndromes, paraneoplastic syndromes, neurologic paraneoplastic syndromes, Lambert-Eaton myasthenic syndrome, Eaton-Lambert syndrome, stiff-man or stiff-person syndrome, encephalomyelitis, allergic encephalomyelitis, encephalomyelitis allergica, experimental allergic encephalomyelitis (EAE), myasthenia gravis, thymoma-associated myasthenia gravis, cerebellar degeneration, neuromyotonia, opsoclonus, opsoclonus myoclonus syndrome (OMS), sensory neuropathy, multifocal motor neuropathy, Sheehan's syndrome, hepatitis, autoimmune hepatitis, chronic hepatitis, lupoid hepatitis, giant cell hepatitis, chronic active hepatitis, autoimmune chronic active hepatitis, lymphoid interstitial pneumonitis (LIP), bronchiolitis obliterans, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis, primary biliary cirrhosis, pneumonocirrhosis, autoimmune enteropathy syndrome, Celiac or Coeliac disease, celiac sprue (gluten enteropathy), refractory sprue, idiopathic sprue, cryoglobulinemia, amyotrophic lateral sclerosis (ALS; Lou Gehrig's disease), coronary artery disease, autoimmune ear disease, autoimmune inner ear disease (AIED), autoimmune hearing loss, polychondritis, refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, monoclonal B cell lymphocytosis, benign monoclonal gammopathy or monoclonal gammopathy of undetermined significance, peripheral neuropathy, paraneoplastic syndrome, channelopathies, epilepsy, migraine, arrhythmia, muscular disorders, deafness, blindness, periodic paralysis, channelopathies of the CNS, autism, inflammatory myopathy, focal or segmental or focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases, autoimmune demyelinating diseases, chronic inflammatory demyelinating polyneuropathy, Dressler's syndrome, alopecia areata, alopecia totalis, CREST syndrome, calcinosis, Raynaud's phenomenon, esophageal dysmotility, sclerodactyly, telangiectasia, male or female autoimmune infertility, anti-spermatozoan antibodies, mixed connective tissue disease, Chagas' disease, rheumatic fever, recurrent abortion, farmer's lung, erythema multiforme, post-cardiotomy syndrome, post myocardial infarction cardiotomy syndrome, Cushing's syndrome, bird-fancier's lung, allergic granulomatous angiitis, benign lymphocytic angiitis, Alport's syndrome, alveolitis, allergic alveolitis, fibrosing alveolitis, interstitial lung disease, transfusion reaction, leprosy, malaria, parasitic diseases, leishmaniasis, kypanosomiasis, schistosomiasis, ascariasis, aspergillosis, Samter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis, chronic cyclitis, heterochronic cyclitis, iridocyclitis (acute or chronic), Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, non-malignant thymoma, vitiligo, toxic-shock syndrome, food poisoning, conditions involving infiltration of T cells, leukocyte-adhesion deficiency, immune responses associated with acute or delayed hypersensitivity mediated by cytokines or T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, sphenoid sinusitis, an eosinophil-related disorder, eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis, central nervous system inflammatory disorders, ocular or orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, emphysema, alopecia areata, adipose tissue inflammation/diabetes type II, obesity-associated adipose tissue inflammation/insulin resistance, endometriosis, and pulmonary hemosiderosis.

Embodiment 141. The method of Embodiment 138, wherein the neurological disease is selected from the group consisting of Parkinson's disease, Huntington disease, Alzheimer's disease, multiple sclerosis, Amyotrophic lateral sclerosis (ALS), spinocerebellar ataxia, dementia, Charcot-Marie-Tooth disease, prion disease, muscular dystrophy, other neurodegenerative disease or condition, attention deficit hyperactivity disorder (ADHD), Tourette Syndrome (TS), schizophrenia, psychosis, depression, bipolar disorder, anxiety disorder, autism spectrum disorder, other neuropsychiatric disorder, and stroke.

Embodiment 142. The method of Embodiment 133 or 134, wherein the disease is a cardiac disease.

Embodiment 143. The method of Embodiments 142, wherein the cardiac disorder is selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, idiopathic cardiomyopathy, other cardiomyopathy, myocardial ischemic reperfusion injury, ventricular dysfunction, heart failure, congestive heart failure, coronary artery disease, end-stage heart disease, atherosclerosis, ischemia, hypertension, restenosis, angina pectoris, rheumatic heart, arterial inflammation, cardiovascular disease, myocardial infarction, myocardial ischemia, congestive heart failure, myocardial infarction, cardiac ischemia, cardiac injury, myocardial ischemia, vascular disease, acquired heart disease, congenital heart disease, atherosclerosis, coronary artery disease, dysfunctional conduction systems, dysfunctional coronary arteries, pulmonary hypertension, cardiac arrhythmias, muscular dystrophy, muscle mass abnormality, muscle degeneration, myocarditis, infective myocarditis, drug- or toxin-induced muscle abnormalities, hypersensitivity myocarditis, and autoimmune endocarditis.

Embodiment 144. The method of any one of Embodiments 96-98, 102-120, 125, 127-131, 132a-132e, 133, and 135-143, wherein the endogenous proliferation gene is selected from the group consisting of AURKB, CDK1, CDC20, RRM2, BIRC5, TOP2A, PTTG1, CCNB1, TPX2, KIF11, SPC25, CENPK, SMC4, TYMS, H2AZ1, TMSB15A, CENPF, and MKI67.

Embodiment 144a. The method of any one of Embodiments 96-98, 102-120, 125, 127-131, 132a-132e, 133, and 135-144, wherein the endogenous proliferation gene is selected from the group consisting of AURKB, RRM2, TOP2A, PTTG1, CCNB1, SPC25, CENPK, SMC4, TYMS, H2AZ1, TMSB15A, CENPF, and MKI67.

Embodiment 144b. The method of any one of Embodiments 96-98, 102-120, 125, 127-131, 132a-132e, 133, and 135-144a, wherein the endogenous proliferation gene is AURKB.

Embodiment 144c. The method of any one of Embodiments 96-98, 102-120, 125, 127-131, 132a-132e, 133, and 135-144a, wherein the endogenous proliferation gene is RRM2.

Embodiment 144d. The method of any one of Embodiments 96-98, 102-120, 125, 127-131, 132a-132e, 133, and 135-144a, wherein the endogenous proliferation gene is TOP2A.

Embodiment 144e. The method of any one of Embodiments 96-98, 102-120, 125, 127-131, 132a-132e, 133, and 135-144a, wherein the endogenous proliferation gene is PTTG1.

Embodiment 144f. The method of any one of Embodiments 96-98, 102-120, 125, 127-131, 132a-132e, 133, and 135-144a, wherein the endogenous proliferation gene is CCNB1.

Embodiment 144g. The method of any one of Embodiments 96-98, 102-120, 125, 127-131, 132a-132e, 133, and 135-144a, wherein the endogenous proliferation gene is SPC25.

Embodiment 144h. The method of any one of Embodiments 96-98, 102-120, 125, 127-131, 132a-132e, 133, and 135-144a, wherein the endogenous proliferation gene is CENPK.

Embodiment 144i. The method of any one of Embodiments 96-98, 102-120, 125, 127-131, 132a-132e, 133, and 135-144a, wherein the endogenous proliferation gene is SMC4.

Embodiment 144j. The method of any one of Embodiments 96-98, 102-120, 125, 127-131, 132a-132e, 133, and 135-144a, wherein the endogenous proliferation gene is TYMS.

Embodiment 144k. The method of any one of Embodiments 96-98, 102-120, 125, 127-131, 132a-132e, 133, and 135-144a, wherein the endogenous proliferation gene is H2AZ1.

Embodiment 144l. The method of Embodiment any one of Embodiments 96-98, 102-120, 125, 127-131, 132a-132e, 133, and 135-144a, wherein the endogenous proliferation gene is TMSB15A.

Embodiment 144m. The method of Embodiment any one of Embodiments 96-98, 102-120, 125, 127-131, 132a-132e, 133, and 135-144a, wherein the endogenous proliferation gene is CENPF.

Embodiment 144n. The method of Embodiment any one of Embodiments 96-98, 102-120, 125, 127-131, 132a-132e, 133, and 135-144a, wherein the endogenous proliferation gene is MKI67.

Embodiment 145. The method of any one of Embodiments any one of Embodiments 96-98, 102-120, 125, 127-131, 132a-132e, 133, and 135-144n, wherein the nucleic acid encoding the selection agent or the detection agent is integrated in the 5′ UTR or the 3′ UTR of the endogenous proliferation gene.

Embodiment 145a. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of AURKB.

Embodiment 145b. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of CDC20.

Embodiment 145c. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of CDK1.

Embodiment 145d. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of RRM2.

Embodiment 145e. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of BIRC5.

Embodiment 145f. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of TOP2A.

Embodiment 145g. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of PTTG1.

Embodiment 145h. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of CCNB1.

Embodiment 145i. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of TPX2.

Embodiment 145j. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of KIF11.

Embodiment 145k. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of SPC25.

Embodiment 145l. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of CENPK.

Embodiment 145m. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of SMC4.

Embodiment 145n. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of TYMS.

Embodiment 145o. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of H2AZ1.

Embodiment 145p. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of TMSB15A.

Embodiment 145q. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of CENPF.

Embodiment 145r. The method of any one of Embodiments 96-98, 102-120, 125, 127-131, 132a-132e, 133, and 135-145, wherein the nucleic acid encoding the selection agent or the detection agent is integrated in a 5′ UTR or a 3′ UTR of MKI67.

Embodiment 146. The method of any one of Embodiments 96-98, 102-120, 125, 127-131, 132a-132e, 133, and 135-144n, wherein the nucleic acid encoding the selection agent or the detection agent is integrated in the exon or intron of the endogenous proliferation gene.

Embodiment 146a. The method of any one of Embodiments 96-98, 102-120, 125, 127-131, 132a-132e, 133, 135-144n, and 146, wherein the nucleic acid encoding the selection agent or the detection agent is integrated in an exon of AURKB, optionally wherein the exon is exon 1, 2, 3, 4, 5, 6, 7, 8, or 9.

Embodiment 146b. The method of any one of Embodiments 96-98, 102-120, 125, 127-131, 132a-132e, 133, 135-144n, and 146, wherein the nucleic acid encoding the selection agent or the detection agent is integrated in an intron of AURKB, optionally wherein the intron is intron 1, 2, 3, 4, 5, 6, 7, or 8.

Embodiment 146c. The method of any one of Embodiments 96-98, 102-120, 125, 127-131, 132a-132e, 133, 135-144n, and 146, wherein the nucleic acid encoding the selection agent or the detection agent is integrated in an exon of CDC20, optionally wherein the exon is exon 1, 2, 3, 4, 5, 6, 7, 8, 9, 10, or 11.

Embodiment 146d. The method of any one of Embodiments 96-98, 102-120, 125, 127-131, 132a-132e, 133, 135-144n, and 146, wherein the nucleic acid encoding the selection agent or the detection agent is integrated in an intron of CDC20, optionally wherein the intron is intron 1, 2, 3, 4, 5, 6, 7, 8, 9, or 10.

Embodiment 146e. The method of any one of Embodiments 96-98, 102-120, 125, 127-131, 132a-132e, 133, 135-144n, and 146, wherein the nucleic acid encoding the selection agent or the detection agent is integrated in an exon of RRM2, optionally wherein the exon is exon 1, 2, 3, 4, 5, 6, 7, 8, 9, or 10.

Embodiment 146f. The method of any one of Embodiments 96-98, 102-120, 125, 127-131, 132a-132e, 133, 135-144n, and 146, wherein the nucleic acid encoding the selection agent or the detection agent is integrated in an intron of RRM2, optionally wherein the intron is intron 1, 2, 3, 4, 5, 6, 7, 8, or 9.

Embodiment 146g. The method of any one of Embodiments 96-98, 102-120, 125, 127-131, 132a-132e, 133, 135-144n, and 146, wherein the nucleic acid encoding the selection agent or the detection agent is integrated in an exon of CDK1, optionally wherein the exon is exon 1, 2, 3, 4, 5, 6, 7, or 8.

Embodiment 146h. The method of any one of Embodiments 96-98, 102-120, 125, 127-131, 132a-132e, 133, 135-144n, and 146, wherein the nucleic acid encoding the selection agent or the detection agent is integrated in an intron of CDK1, optionally wherein the intron is intron 1, 2, 3, 4, 5, 6, or 7.

Embodiment 146i. The method of any one of Embodiments 96-98, 102-120, 125, 127-131, 132a-132e, 133, 135-144n, and 146, wherein the nucleic acid encoding the selection agent or the detection agent is integrated in an exon of BIRC5, optionally wherein the exon is exon 1, 2, 3, or 4.

Embodiment 146j. The method of any one of Embodiments 96-98, 102-120, 125, 127-131, 132a-132e, 133, 135-144n, and 146, wherein the nucleic acid encoding the selection agent or the detection agent is integrated in an intron of BIRC5, optionally wherein the intron is intron 1, 2, or 3.

Embodiment 146k. The method of any one of Embodiments 96-98, 102-120, 125, 127-131, 132a-132e, 133, 135-144n, and 146, wherein the nucleic acid encoding the selection agent or the detection agent is integrated in an exon of TOP2A, optionally wherein the exon is exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35.

Embodiment 146l. The method of any one of Embodiments 96-98, 102-120, 125, 127-131, 132a-132e, 133, 135-144n, and 146, wherein the nucleic acid encoding the selection agent or the detection agent is integrated in an intron of TOP2A, optionally wherein the intron is intron 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34.

Embodiment 146m. The method of any one of Embodiments 96-98, 102-120, 125, 127-131, 132a-132e, 133, 135-144n, and 146, wherein the nucleic acid encoding the selection agent or the detection agent is integrated in an exon of PTTG1, optionally wherein the exon is exon 1, 2, 3, 4, 5, or 6.

Embodiment 146n. The method of any one of Embodiments 96-98, 102-120, 125, 127-131, 132a-132e, 133, 135-144n, and 146, wherein the nucleic acid encoding the selection agent or the detection agent is integrated in an intron of PTTG1, optionally wherein the intron is intron 1, 2, 3, 4, or 5.

Embodiment 146o. The method of any one of Embodiments 96-98, 102-120, 125, 127-131, 132a-132e, 133, 135-144n, and 146, wherein the nucleic acid encoding the selection agent or the detection agent is integrated in an exon of CCNB1, optionally wherein the exon is exon 1, 2, 3, 4, 5, 6, 7, 8, or 9.

Embodiment 146p. The method of any one of Embodiments 96-98, 102-120, 125, 127-131, 132a-132e, 133, 135-144n, and 146, wherein the nucleic acid encoding the selection agent or the detection agent is integrated in an intron of CCNB1, optionally wherein the intron is intron 1, 2, 3, 4, 5, 6, 7, or 8.

Embodiment 146q. The method of any one of Embodiments 96-98, 102-120, 125, 127-131, 132a-132e, 133, 135-144n, and 146, wherein the nucleic acid encoding the selection agent or the detection agent is integrated in an exon of TPX2, optionally wherein the exon is exon 1, 2, 3, 4, 5, 6, 7, 8, 9, 10, 11, 12, 13, 14, 15, 16, 17, or 18.

Embodiment 146r. The method of any one of Embodiments 96-98, 102-120, 125, 127-131, 132a-132e, 133, 135-144n, and 146, wherein the nucleic acid encoding the selection agent or the detection agent is integrated in an intron of TPX2, optionally wherein the intron is intron 1, 2, 3, 4, 5, 6, 7, 8, 9, 10, 11, 12, 13, 14, 15, 16, or 17.

Embodiment 146s. The method of any one of Embodiments 96-98, 102-120, 125, 127-131, 132a-132e, 133, 135-144n, and 146, wherein the nucleic acid encoding the selection agent or the detection agent is integrated in an exon of KIF11, optionally wherein the exon is exon 1, 2, 3, 4, 5, 6, 7, 8, 9, 10, 11, 12, 13, 14, 15, 16, 17, 18, 19, 20, 21, or 22.

Embodiment 146t. The method of any one of Embodiments 96-98, 102-120, 125, 127-131, 132a-132e, 133, 135-144n, and 146, wherein the nucleic acid encoding the selection agent or the detection agent is integrated in an intron of KIF11, optionally wherein the intron is intron 1, 2, 3, 4, 5, 6, 7, 8, 9, 10, 11, 12, 13, 14, 15, 16, 17, 18, 19, 20, or 21.

Embodiment 146u. The method of any one of Embodiments 96-98, 102-120, 125, 127-131, 132a-132e, 133, 135-144n, and 146, wherein the nucleic acid encoding the selection agent or the detection agent is integrated in an exon of SPC25, optionally wherein the exon is exon 1, 2, 3, 4, 5, 6, or 7.

Embodiment 146v. The method of any one of Embodiments 96-98, 102-120, 125, 127-131, 132a-132e, 133, 135-144n, and 146, wherein the nucleic acid encoding the selection agent or the detection agent is integrated in an intron of SPC25, optionally wherein the intron is intron 1, 2, 3, 4, 5, or 6.

Embodiment 146w. The method of any one of Embodiments 96-98, 102-120, 125, 127-131, 132a-132e, 133, 135-144n, and 146, wherein the nucleic acid encoding the selection agent or the detection agent is integrated in an exon of CENPK, optionally wherein the exon is exon 1, 2, 3, 4, 5, 6, 7, 8, 9, 10, or 11.

Embodiment 146x. The method of any one of Embodiments 96-98, 102-120, 125, 127-131, 132a-132e, 133, 135-144n, and 146, wherein the nucleic acid encoding the selection agent or the detection agent is integrated in an intron of CENPK, optionally wherein the intron is intron 1, 2, 3, 4, 5, 6, 7, 8, 9, or 10.

Embodiment 146y. The method of any one of Embodiments 96-98, 102-120, 125, 127-131, 132a-132e, 133, 135-144n, and 146, wherein the nucleic acid encoding the selection agent or the detection agent is integrated in an exon of SMC4, optionally wherein the exon is exon 1, 2, 3, 4, 5, 6, 7, 8, 9, 10, 11, 12, 13, 14, 15, 16, 17, 18, 19, 20, 21, 22, 23, or 24.

Embodiment 146z. The method of any one of Embodiments 96-98, 102-120, 125, 127-131, 132a-132e, 133, 135-144n, and 146, wherein the nucleic acid encoding the selection agent or the detection agent is integrated in an intron of SMC4, optionally wherein the intron is intron 1, 2, 3, 4, 5, 6, 7, 8, 9, 10, 11, 12, 13, 14, 15, 16, 17, 18, 19, 20, 21, 22, or 23.

Embodiment 146aa. The method of any one of Embodiments 96-98, 102-120, 125, 127-131, 132a-132e, 133, 135-144n, and 146, wherein the nucleic acid encoding the selection agent or the detection agent is integrated in an exon of TYMS, optionally wherein the exon is exon 1, 2, 3, 4, 5, 6, or 7.

Embodiment 146bb. The method of any one of Embodiments 96-98, 102-120, 125, 127-131, 132a-132e, 133, 135-144n, and 146, wherein the nucleic acid encoding the selection agent or the detection agent is integrated in an intron of TYMS, optionally wherein the intron is intron 1, 2, 3, 4, 5, or 6.

Embodiment 146cc. The method of any one of Embodiments 96-98, 102-120, 125, 127-131, 132a-132e, 133, 135-144n, and 146, wherein the nucleic acid encoding the selection agent or the detection agent is integrated in an exon of H2AZ1, optionally wherein the exon is exon 1, 2, 3, 4, or 5.

Embodiment 146dd. The method of any one of Embodiments 96-98, 102-120, 125, 127-131, 132a-132e, 133, 135-144n, and 146, wherein the nucleic acid encoding the selection agent or the detection agent is integrated in an intron of H2AZ1, optionally wherein the intron is intron 1, 2, 3, or 4.

Embodiment 146ee. The method of any one of Embodiments 96-98, 102-120, 125, 127-131, 132a-132e, 133, 135-144n, and 146, wherein the nucleic acid encoding the selection agent or the detection agent is integrated in an exon of TMSB15A, optionally wherein the exon is exon 1, 2, or 3.

Embodiment 146ff. The method of any one of Embodiments 96-98, 102-120, 125, 127-131, 132a-132e, 133, 135-144n, and 146, wherein the nucleic acid encoding the selection agent or the detection agent is integrated in an intron of TMSB15A, optionally wherein the intron is intron 1 or 2.

Embodiment 146gg. The method of any one of Embodiments 96-98, 102-120, 125, 127-131, 132a-132e, 133, 135-144n, and 146, wherein the nucleic acid encoding the selection agent or the detection agent is integrated in an exon of CENPF, optionally wherein the exon is exon 1, 2, 3, 4, 5, 6, 7, 8, 9, 10, 11, 12, 13, 14, 15, 16, 17, 18, 19, or 20.

Embodiment 146hh. The method of any one of Embodiments 96-98, 102-120, 125, 127-131, 132a-132e, 133, 135-144n, and 146, wherein the nucleic acid encoding the selection agent or the detection agent is integrated in an intron of CENPF, optionally wherein the intron is intron 1, 2, 3, 4, 5, 6, 7, 8, 9, 10, 11, 12, 13, 14, 15, 16, 17, 18, or 19.

146ii. The method of any one of Embodiments 96-98, 102-120, 125, 127-131, 132a-132e, 133, 135-144n, and 146, wherein the nucleic acid encoding the selection agent or the detection agent is integrated in an exon of MKI67, optionally wherein the exon is exon 1, 2, 3, 4, 5, 6, 7, 8, 9, 10, 11, 12, 13, 14, or 15.

146jj. The method of any one of Embodiments 96-98, 102-120, 125, 127-131, 132a-132e, 133, 135-144n, and 146, wherein the nucleic acid encoding the selection agent or the detection agent is integrated in an intron of MKI67, optionally wherein the intron is intron 1, 2, 3, 4, 5, 6, 7, 8, 9, 10, 11, 12, 13, or 14.

Embodiment 147. The method of any one of Embodiments 96-98, 102-120, 125, 127-131, 132a-132e, 133, and 135-146jj, wherein expression of the nucleic acid encoding the selection agent or a detection agent is regulated by the endogenous promoter and/or regulatory elements of the endogenous proliferation gene.

Embodiment 148. The method of any one of Embodiments 96-98, 102-120, 125, 127-131, 132a-132e, 133, 135-146jj, and 147, wherein following integration, the endogenous proliferation gene locus comprises i) nucleic acid encoding the proliferation gene, ii) an internal ribosomal entry site or a self-cleaving RNA site, and iii) the nucleic acid encoding the selection agent or a detection agent.

Embodiment 149. The method of any one of Embodiments 96-98, 102-120, 125, 127-131, 132a-132e, 133, 135-148, wherein the selection agent or the detection agent is expressed in proliferating cells.

Embodiment 150. The method of any one of Embodiments 96-98, 102-120, 125, 127-131, 132a-132e, 133, 135-149, wherein the selection agent or the detection agent is not expressed in non-proliferating cells.

Embodiment 151. The method of any one of Embodiments 96-98, 102-120, 125, 127-131, 132a-132e, 133, 135-150, wherein the selection agent or the detection agent is expressed in partially differentiated cells.

Embodiment 152. The method of any one of Embodiments 96-98, 102-120, 125, 127-131, 132a-132e, 133, 135-151, wherein the selection agent or the detection agent is expressed in an intermediate cell type made during differentiation.

Embodiment 153. The method of any one of Embodiments 96-98, 102-120, 125, 127-131, 132a-132e, 133, 135-152, wherein the selection agent or the detection agent is not expressed in differentiated cells.

Embodiment 154. The method of any one of Embodiments 96-98, 102-120, 125, 127-131, 132a-132e, 133, 135-153, wherein the selection agent or the detection agent is not expressed in a therapeutic cell.

Embodiment 155. The method of any one of Embodiments 96-98, 102-120, 125, 127-131, 132a-132e, 133, 135-154, wherein the cell is selected from the group consisting of a pluripotent stem cell, an induced pluripotent stem cell, a primary cell, a progenitor cell, an embryonic stem cell, a hematopoietic stem cell, a mesenchymal stem cell, an epithelial stem cell, a germline stem cell, a mammary stem cell, an olfactory adult stem cell, a hair follicle stem cell, a multipotent stem cell, an amniotic stem cell, a cord blood stem cell, a neural stem cell, a somatic stem cell, a totipotent stem cell, a fibroblast, a monocytic precursor, an exocrine cell, a pancreatic progenitor, an endocrine progenitor, a hepatoblast, a myoblast, a preadipocyte, a chondrocyte, a bone cell, a synovial cell, a tendon cell, a ligament cell, a meniscus cell, an adipose cell, a dendritic cell, a natural killer cell, a muscle cell, an erythroid-megakaryocytic cell, an eosinophil, an islet beta cell, a neuron, a cardiomyocyte, a blood cell, an exocrine progenitor, a ductal cell, an acinar cell, an alpha cell, a beta cell, a delta cell, a cholangiocyte, a brown adipocyte, a cardiac muscle cell, a PP cell, an epidermal keratinocyte, an epithelial cell, a germ cell, a skeletal joint synovium cell, a periosteum cell, a bone cell, a perichondrium cell, a pericardium cell, a meningeal cell, a keratinocyte precursor cell, a keratinocyte stem cell, a pericyte, a glial cell, an ependymal cell, a serosal cell, a heart cell, a brain cell, a spinal cord cell, a lung cell, a pancreas cell, a bladder cell, a bone marrow cell, a spleen cell, an intestine cell, a stomach cell, an islet cell, a pancreatic islet cell, an immune cell, a B cell, a T cell, a natural killer (NK) cell, a natural killer T (NKT) cell, a macrophage, an endothelial cell, a muscle cell, a smooth muscle cell, a skeletal muscle cell, a dopaminergic neuron, a retinal pigmented epithelium cell, an optic cell, a hepatocyte, a thyroid cell, a skin cell, a glial progenitor cell, a neural cell, a stem cell, an endothelial stem cell, an adipose stem cell, an adipose progenitor cell, a lung stem cell, a lung progenitor cell, a neural progenitor cell, a T cell, a CAR-T cell, a CD14+ cell, a dendritic cell, a PBMC cell, an exocrine secretory epithelial cell, a thyroid epithelial cell, a keratinizing epithelial cell, a gall bladder epithelial cell, a surface epithelial cell, a kidney cell, a primary T cell, a CD3+ T cell, a CD4+ T cell, a CD8+ T cell, a naive T cell, a regulatory T (Treg) cell, a non-regulatory T cell, a Th1 cell, a Th2 cell, a Th9 cell, a Th17 cell, a T-follicular helper (Tfh) cell, a cytotoxic T lymphocyte (CTL), an effector T (Teff) cell, a central memory T (Tcm) cell, an effector memory T (Tem) cell, a TEMRA cells, a tissue-resident memory (Trm) cell, a virtual memory T cell, an innate memory T cell, a memory stem cell (Tsc), a T6 T cell, a helper T cell, a central memory T cell, an effector memory T cell, an effector memory RA T cell, a tissue infiltrating lymphocyte, a capillary endothelial cell, a vascular endothelial cell, an aortic endothelial cell, an arterial endothelial cell, a venous endothelial cell, a renal endothelial cell, a brain endothelial cell, a liver endothelial cell, a gamma cell, an epsilon cell, hematopoietic progenitor cell, a nodal cardiomyocyte, a conducting cardiomyocyte, a working cardiomyocyte, a cardiomyocyte precursor cell, a cardiomyocyte progenitor cell, a cardiac stem cell, an atrial cardiac stem cell, a ventricular cardiac stem cell, an epicardial cell, a vascular endothelial cell, an endocardial endothelial cell, a cardiac valve interstitial cell, a cardiac pacemaker cell, a neuroectodermal cell, a neuronal cell, a neuroendocrine cell, a cholinergic cell, a serotonergic (5-HT) cell, a glutamatergic cell, a GABAergic cell, an adrenergic cell, a noradrenergic cell, a sympathetic neuronal cell, a parasympathetic neuronal cell, a sympathetic peripheral neuronal cell, an astrocyte, an oligodendrocyte, an ependymal cell, a radial glia cell, a Schwann cell, a cerebral endothelial cells (ECs), a neuronal stem cell, a neuronal progenitor cell, a white blood cell, a red blood cell, a platelet cell, a tumor cell, an enterochromaffin cell, a mesenchymal fibroblast, an osteoblast, a stromal cell, and any combination thereof.

Embodiment 156. The method of Embodiment 155, wherein the cell is selected from the group consisting of a pancreatic islet cell, an alpha cell, a beta cell, a gamma cell, a delta cell, an epsilon cell, a T cell, a neuron, a glial cell, a cardiomyocyte, a retinal pigmented epithelial cell, a hematopoietic progenitor cell, a natural killer cell, an endothelial cell, and a lung cell.

Embodiment 157. The method of any one of Embodiments 99-116g, 121-124, 126-130, 132-132d, 132f, and 134-143, wherein the off-target cell marker gene is selected from the group consisting of a pluripotent stem cell marker gene, an induced pluripotent stem cell marker gene, a primary cell marker gene, a progenitor cell marker gene, an embryonic stem cell marker gene, a hematopoietic stem cell marker gene, a mesenchymal stem cell marker gene, an epithelial stem cell marker gene, a germline stem cell marker gene, a mammary stem cell marker gene, an olfactory adult stem cell marker gene, a hair follicle stem cell marker gene, a multipotent stem cell marker gene, an amniotic stem cell marker gene, a cord blood stem cell marker gene, a neural stem cell marker gene, a somatic stem cell marker gene, a totipotent stem cell marker gene, a fibroblast marker gene, a monocytic precursor marker gene, an exocrine cell marker gene, a pancreatic progenitor marker gene, an endocrine progenitor marker gene, a hepatoblast marker gene, a myoblast marker gene, a preadipocyte marker gene, a chondrocyte marker gene, a synovial cell marker gene, a tendon cell marker gene, a ligament cell marker gene, a meniscus cell marker gene, an adipose cell marker gene, a dendritic cell marker gene, a natural killer cell marker gene, a muscle cell marker gene, an erythroid-megakaryocytic cell marker gene, an eosinophil marker gene, an islet beta cell marker gene, a neuron marker gene, a cardiomyocyte marker gene, a blood cell marker gene, an exocrine progenitor marker gene, a ductal cell marker gene, an acinar cell marker gene, an alpha cell marker gene, a beta cell marker gene, a delta cell marker gene, a cholangiocyte marker gene, a brown adipocyte marker gene, a cardiac muscle cell marker gene, a PP cell marker gene, an epidermal keratinocyte marker gene, an epithelial cell marker gene, a germ cell marker gene, a skeletal joint synovium cell marker gene, a periosteum cell marker gene, a bone cell marker gene, a perichondrium cell marker gene, a pericardium cell marker gene, a meningeal cell marker gene, a keratinocyte precursor cell marker gene, a keratinocyte stem cell marker gene, a pericyte marker gene, a glial cell marker gene, an ependymal cell marker gene, a serosal cell marker gene, a heart cell marker gene, a brain cell marker gene, a spinal cord cell marker gene, a lung cell marker gene, a pancreas cell marker gene, a bladder cell marker gene, a bone marrow cell marker gene, a spleen cell marker gene, an intestine cell marker gene, a stomach cell marker gene, an islet cell marker gene, a pancreatic islet cell marker gene, an immune cell marker gene, a B cell marker gene, a T cell marker gene, a natural killer (NK) cell marker gene, a natural killer T (NKT) cell marker gene, a macrophage marker gene, an endothelial cell marker gene, a muscle cell marker gene, a smooth muscle cell marker gene, a skeletal muscle cell marker gene, a dopaminergic neuron marker gene, a retinal pigmented epithelium cell marker gene, an optic cell marker gene, a hepatocyte marker gene, a thyroid cell marker gene, a skin cell marker gene, a glial progenitor cell marker gene, a neural cell marker gene, a stem cell marker gene, an endothelial stem cell marker gene, an adipose stem cell marker gene, an adipose progenitor cell marker gene, a lung stem cell marker gene, a lung progenitor cell marker gene, a neural progenitor cell marker gene, a T cell marker gene, a CAR-T cell marker gene, a CD14+ cell marker gene, a dendritic cell marker gene, a PBMC cell marker gene, an exocrine secretory epithelial cell marker gene, a thyroid epithelial cell marker gene, a keratinizing epithelial cell marker gene, a gall bladder epithelial cell marker gene, a surface epithelial cell marker gene, a kidney cell marker gene, a primary T cell marker gene, a CD3+ T cell marker gene, a CD4+ T cell marker gene, a CD8+ T cell marker gene, a naive T cell marker gene, a regulatory T (Treg) cell marker gene, a non-regulatory T cell marker gene, a Th1 cell marker gene, a Th2 cell marker gene, a Th9 cell marker gene, a Th17 cell marker gene, a T-follicular helper (Tfh) cell marker gene, a cytotoxic T lymphocyte (CTL) marker gene, an effector T (Teff) cell marker gene, a central memory T (Tcm) cell marker gene, an effector memory T (Tem) cell marker gene, a TEMRA cell marker gene, a tissue-resident memory (Trm) cell marker gene, a virtual memory T cell marker gene, an innate memory T cell marker gene, a memory stem cell (Tsc) marker gene, a T6 T cell marker gene, a helper T cell marker gene, a central memory T cell marker gene, an effector memory T cell marker gene, an effector memory RA T cell marker gene, a tissue infiltrating lymphocyte marker gene, a capillary endothelial cell marker gene, a vascular endothelial cell marker gene, an aortic endothelial cell marker gene, an arterial endothelial cell marker gene, a venous endothelial cell marker gene, a renal endothelial cell marker gene, a brain endothelial cell marker gene, a liver endothelial cell marker gene, a gamma cell marker gene, an epsilon cell marker gene, hematopoietic progenitor cell marker gene, a nodal cardiomyocyte marker gene, a conducting cardiomyocyte marker gene, a working cardiomyocyte marker gene, a cardiomyocyte precursor cell marker gene, a cardiomyocyte progenitor cell marker gene, a cardiac stem cell marker gene, an atrial cardiac stem cell marker gene, a ventricular cardiac stem cell marker gene, an epicardial cell marker gene, a vascular endothelial cell marker gene, an endocardial endothelial cell marker gene, a cardiac valve interstitial cell marker gene, a cardiac pacemaker cell marker gene, a neuroectodermal cell marker gene, a neuronal cell marker gene, a neuroendocrine cell marker gene, a cholinergic cell marker gene, a serotonergic (5-HT) cell marker gene, a glutamatergic cell marker gene, a GABAergic cell marker gene, an adrenergic cell marker gene, a noradrenergic cell marker gene, a sympathetic neuronal cell marker gene, a parasympathetic neuronal cell marker gene, a sympathetic peripheral neuronal cell marker gene, an astrocyte marker gene, an oligodendrocyte marker gene, an ependymal cell marker gene, a radial glia cell marker gene, a Schwann cell marker gene, a cerebral endothelial cells (ECs) marker gene, a neuronal stem cell marker gene, a neuronal progenitor cell marker gene, a white blood cell marker gene, a red blood cell marker gene, a platelet cell marker gene, a tumor cell marker gene, an enterochromaffin cell marker gene, a mesenchymal fibroblast marker gene, an osteoblast marker gene, a stromal cell marker gene, and any combination thereof.

Embodiment 158. The method of Embodiment 157, wherein the off-target cell marker gene is selected from the group consisting of a pluripotency cell marker gene, a tumorigenic cell marker gene, a ductal marker gene, an enterochromaffin marker gene, a neural marker gene, acinar marker gene, intestinal marker gene, endothelial marker gene, mesenchymal fibroblast marker gene, muscle marker gene, osteoblast marker gene, and stromal marker gene.

Embodiment 159. The method of any one of Embodiments 99-116g, 121-124, 126-130, 132-132d, 132f, and 134-143, 157, and 158, wherein the off-target cell marker gene is selected from the group consisting of ANXA1, KRT19, CTSC, DSC2, ARHGAP29, KRT18, KRT8, CD9, PLK2, and KRT17.

Embodiment 159a. The method of any one of Embodiments 99-116g, 121-124, 126-130, 132-132d, 132f, and 134-143, and 157-159, wherein the off-target cell marker gene is ANXA1.

Embodiment 159b. The method of any one of Embodiments 99-116g, 121-124, 126-130, 132-132d, 132f, and 134-143, and 157-159, wherein the off-target cell marker gene is KRT19.

Embodiment 159c. The method of any one of Embodiments 99-116g, 121-124, 126-130, 132-132d, 132f, and 134-143, and 157-159, wherein the off-target cell marker gene is CTSC.

Embodiment 159d. The method of any one of Embodiments 99-116g, 121-124, 126-130, 132-132d, 132f, and 134-143, and 157-159, wherein the off-target cell marker gene is DSC2.

Embodiment 159e. The method of any one of Embodiments 99-116g, 121-124, 126-130, 132-132d, 132f, and 134-143, and 157-159, wherein the off-target cell marker gene is ARHGAP29.

Embodiment 159f. The method of any one of Embodiments 99-116g, 121-124, 126-130, 132-132d, 132f, and 134-143, and 157-159, wherein the off-target cell marker gene is KRT18.

Embodiment 159g. The method of any one of Embodiments 99-116g, 121-124, 126-130, 132-132d, 132f, and 134-143, and 157-159, wherein the off-target cell marker gene is KRT8.

Embodiment 159h. The method of any one of Embodiments 99-116g, 121-124, 126-130, 132-132d, 132f, and 134-143, and 157-159, wherein the off-target cell marker gene is CD9.

Embodiment 159i. The method of any one of Embodiments 99-116g, 121-124, 126-130, 132-132d, 132f, and 134-143, and 157-159, wherein the off-target cell marker gene is PLK2.

Embodiment 159j. The method of any one of Embodiments 99-116g, 121-124, 126-130, 132-132d, 132f, and 134-143, and 157-159, wherein the off-target cell marker gene is KRT17.

Embodiment 160. The method of any one of Embodiments 99-116g, 121-124, 126-130, 132-132d, 132f, and 134-143, and 157-159j, wherein the nucleic acid encoding the selection agent or the detection agent is integrated in the 5′ UTR or 3′ UTR of the off-target cell marker gene.

Embodiment 160a. The method of any one of Embodiments 99-116g, 121-124, 126-130, 132-132d, 132f, and 134-143, and 157-160, wherein the nucleic acid encoding the selection agent or the detection agent is integrated in a 5′ UTR or a 3′ UTR of ANXA1.

Embodiment 160b. The method of any one of Embodiments 99-116g, 121-124, 126-130, 132-132d, 132f, and 134-143, and 157-160, wherein the nucleic acid encoding the selection agent or the detection agent is integrated in a 5′ UTR or a 3′ UTR of KRT19.

Embodiment 160c. The method of any one of Embodiments 99-116g, 121-124, 126-130, 132-132d, 132f, and 134-143, and 157-160, wherein the nucleic acid encoding the selection agent or the detection agent is integrated in a 5′ UTR or a 3′ UTR of CTSC.

Embodiment 160d. The method of any one of Embodiments 99-116g, 121-124, 126-130, 132-132d, 132f, and 134-143, and 157-160, wherein the nucleic acid encoding the selection agent or the detection agent is integrated in a 5′ UTR or a 3′ UTR of DSC2.

Embodiment 160e. The method of any one of Embodiments 99-116g, 121-124, 126-130, 132-132d, 132f, and 134-143, and 157-160, wherein the nucleic acid encoding the selection agent or the detection agent is integrated in a 5′ UTR or a 3′ UTR of ARHGAP29.

Embodiment 160f. The method of any one of Embodiments 99-116g, 121-124, 126-130, 132-132d, 132f, and 134-143, and 157-160, wherein the nucleic acid encoding the selection agent or the detection agent is integrated in a 5′ UTR or a 3′ UTR of KRT18.

Embodiment 160g. The method of any one of Embodiments 99-116g, 121-124, 126-130, 132-132d, 132f, and 134-143, and 157-160, wherein the nucleic acid encoding the selection agent or the detection agent is integrated in a 5′ UTR or a 3′ UTR of KRT8.

Embodiment 160h. The method of any one of Embodiments 99-116g, 121-124, 126-130, 132-132d, 132f, and 134-143, and 157-160, wherein the nucleic acid encoding the selection agent or the detection agent is integrated in a 5′ UTR or a 3′ UTR of CD9.

Embodiment 160i. The method of any one of Embodiments 99-116g, 121-124, 126-130, 132-132d, 132f, and 134-143, and 157-160, wherein the nucleic acid encoding the selection agent or the detection agent is integrated in a 5′ UTR or a 3′ UTR of PLK2.

Embodiment 160j. The method of any one of Embodiments 99-116g, 121-124, 126-130, 132-132d, 132f, and 134-143, and 157-160, wherein the nucleic acid encoding the selection agent or the detection agent is integrated in a 5′ UTR or a 3′ UTR of KRT17.

Embodiment 161. The method of any one of Embodiments 99-116g, 121-124, 126-130, 132-132d, 132f, and 134-143, and 157-159j, wherein the nucleic acid encoding the selection agent or the detection agent is integrated in the exon or intron of the off-target cell marker gene.

Embodiment 161a. The method of any one of Embodiments 99-116g, 121-124, 126-130, 132-132d, 132f, and 134-143, 157-159j, and 161, wherein the nucleic acid encoding the selection agent or the detection agent is integrated in an exon of ANXA1, optionally wherein the exon is exon 1, 2, 3, 4, 5, 6, 7, 8, 9, 10, 11, 12, or 13.

Embodiment 161b. The method of any one of Embodiments 99-116g, 121-124, 126-130, 132-132d, 132f, and 134-143, 157-159j, and 161, wherein the nucleic acid encoding the selection agent or the detection agent is integrated in an intron of ANXA1, optionally wherein the intron is intron 1, 2, 3, 4, 5, 6, 7, 8, 9, 10, 11, or 12.

Embodiment 161c. The method of any one of Embodiments 99-116g, 121-124, 126-130, 132-132d, 132f, and 134-143, 157-159j, and 161, wherein the nucleic acid encoding the selection agent or the detection agent is integrated in an exon of KRT19, optionally wherein the exon is exon 1, 2, 3, 4, 5, or 6.

Embodiment 161d. The method of any one of Embodiments 99-116g, 121-124, 126-130, 132-132d, 132f, and 134-143, 157-159j, and 161, wherein the nucleic acid encoding the selection agent or the detection agent is integrated in an intron of KRT19, optionally wherein the intron is intron 1, 2, 3, 4, or 5.

Embodiment 161e. The method of any one of Embodiments 99-116g, 121-124, 126-130, 132-132d, 132f, and 134-143, 157-159j, and 161, wherein the nucleic acid encoding the selection agent or the detection agent is integrated in an exon of CTSC, optionally wherein the exon is exon 1, 2, 3, 4, 5, 6, or 7.

Embodiment 161f. The method of any one of Embodiments 99-116g, 121-124, 126-130, 132-132d, 132f, and 134-143, 157-159j, and 161, wherein the nucleic acid encoding the selection agent or the detection agent is integrated in an intron of CTSC, optionally wherein the intron is intron 1, 2, 3, 4, 5, or 6.

Embodiment 161g. The method of any one of Embodiments 99-116g, 121-124, 126-130, 132-132d, 132f, and 134-143, 157-159j, and 161, wherein the nucleic acid encoding the selection agent or the detection agent is integrated in an exon of DSC2, optionally wherein the exon is exon 1, 2, 3, 4, 5, 6, 7, 8, 9, 10, 11, 12, 13, 14, 15, or 16.

Embodiment 161h. The method of any one of Embodiments 99-116g, 121-124, 126-130, 132-132d, 132f, and 134-143, 157-159j, and 161, wherein the nucleic acid encoding the selection agent or the detection agent is integrated in an intron of DSC2, optionally wherein the intron is intron 1, 2, 3, 4, 5, 6, 7, 8, 9, 10, 11, 12, 13, 14, or 15.

Embodiment 161i. The method of any one of Embodiments 99-116g, 121-124, 126-130, 132-132d, 132f, and 134-143, 157-159j, and 161, wherein the nucleic acid encoding the selection agent or the detection agent is integrated in an exon of ARHGAP29, optionally wherein the exonis exon 1, 2, 3, 4, 5, 6, 7, 8, 9, 10, 11, 12, 13, 14, 15, 16, 17, 18, 19, 20, 21, 22, or 23.

Embodiment 161j. The method of any one of Embodiments 99-116g, 121-124, 126-130, 132-132d, 132f, and 134-143, 157-159j, and 161, wherein the nucleic acid encoding the selection agent or the detection agent is integrated in an intron of ARHGAP29, optionally wherein the intron is intron 1, 2, 3, 4, 5, 6, 7, 8, 9, 10, 11, 12, 13, 14, 15, 16, 17, 18, 19, 20, 21, or 22.

Embodiment 161k. The method of any one of Embodiments 99-116g, 121-124, 126-130, 132-132d, 132f, and 134-143, 157-159j, and 161, wherein the nucleic acid encoding the selection agent or the detection agent is integrated in an exon of KRT18, optionally wherein the exon is exon 1, 2, 3, 4, 5, 6, or 7.

Embodiment 161l. The method of any one of Embodiments 99-116g, 121-124, 126-130, 132-132d, 132f, and 134-143, 157-159j, and 161, wherein the nucleic acid encoding the selection agent or the detection agent is integrated in an intron of KRT18, optionally wherein the intron is intron 1, 2, 3, 4, 5, or 6.

Embodiment 161m. The method of any one of Embodiments 99-116g, 121-124, 126-130, 132-132d, 132f, and 134-143, 157-159j, and 161, wherein the nucleic acid encoding the selection agent or the detection agent is integrated in an exon of KRT8, optionally wherein the exon is exon 1, 2, 3, 4, 5, 6, 7, or 8.

Embodiment 161n. The method of any one of Embodiments 99-116g, 121-124, 126-130, 132-132d, 132f, and 134-143, 157-159j, and 161, wherein the nucleic acid encoding the selection agent or the detection agent is integrated in an intron of KRT8, optionally wherein the intron is intron 1, 2, 3, 4, 5, 6, or 7.

Embodiment 161o. The method of any one of Embodiments 99-116g, 121-124, 126-130, 132-132d, 132f, and 134-143, 157-159j, and 161, wherein the nucleic acid encoding the selection agent or the detection agent is integrated in an exon of CD9, optionally wherein the exon is exon 1, 2, 3, 4, 5, 6, 7, or 8.

Embodiment 161p. The method of any one of Embodiments 99-116g, 121-124, 126-130, 132-132d, 132f, and 134-143, 157-159j, and 161, wherein the nucleic acid encoding the selection agent or the detection agent is integrated in an intron of CD9, optionally wherein the intron is intron 1, 2, 3, 4, 5, 6, or 7.

Embodiment 161q. The method of any one of Embodiments 99-116g, 121-124, 126-130, 132-132d, 132f, and 134-143, 157-159j, and 161, wherein the nucleic acid encoding the selection agent or the detection agent is integrated in an exon of PLK2, optionally wherein the exon is exon 1, 2, 3, 4, 5, 6, 7, 8, 9, 10, 11, 12, 13, or 14.

Embodiment 161r. The method of any one of Embodiments 99-116g, 121-124, 126-130, 132-132d, 132f, and 134-143, 157-159j, and 161, wherein the nucleic acid encoding the selection agent or the detection agent is integrated in an intron of PLK2, optionally wherein the intron is intron 1, 2, 3, 4, 5, 6, 7, 8, 9, 10, 11, 12, or 13.

Embodiment 161s. The method of any one of Embodiments 99-116g, 121-124, 126-130, 132-132d, 132f, and 134-143, 157-159j, and 161, wherein the nucleic acid encoding the selection agent or the detection agent is integrated in an exon of KRT17, optionally wherein the exon is exon 1, 2, 3, 4, 5, 6, 7, or 8.

Embodiment 161t. The method of any one of Embodiments 99-116g, 121-124, 126-130, 132-132d, 132f, and 134-143, 157-159j, and 161, wherein the nucleic acid encoding the selection agent or the detection agent is integrated in an intron of KRT17, optionally wherein the intron is intron 1, 2, 3, 4, 5, 6, or 7.

Embodiment 162. The method of any one of Embodiments 99-116g, 121-124, 126-130, 132-132d, 132f, and 134-143, and 157-161t, wherein the off-target cell marker gene is not a beta cell marker gene, a T cell marker gene, a neuronal cell marker gene, a glial cell marker gene, a cardiac cell marker gene, a retinal pigment epithelium (RPE) cell marker gene, a hematopoietic progenitor cell marker gene, a natural killer cell marker gene, an endothelial cell marker gene, or a lung cell marker gene.

Embodiment 163. A kit comprising a cell comprising the cell of any one of Embodiments 1-26a and 60-60r and instructions for use.

Embodiment 164. A kit comprising a cell comprising the cell of any one of Embodiments 27-59 and 60-60r and instructions for use.

Embodiment 165. The kit of Embodiment 163 or 164, wherein the kit further comprises an inducer.

Embodiment 166. The cell of any one of Embodiments 1-60r, the method of any one of Embodiments 63-162, and the kit of any one of Embodiments 163-165, wherein the inducer is selected from the group consisting of rimiducid (AP1903), AP20187, rapamycin, 5-fluorocytosine, ganciclovir, CB 1954, 6-methylpurine deoxyriboside, fludarabine, indole-3-acetic acid (IAA), tetracycline, doxycycline, tamoxifen, cumate, FKCsA, abscisic acid (ABA), riboswitch, ecdysone, tryptophan, arabinose, isopropyl β-d-1-thiogalactopyranoside (IPTG), asunaprevir, grazoprevir, dTAG-13, lenalidomide, anti-MHC-I antibodies, anti-MHC-II antibodies, anti-CCR4 antibodies, anti-CD16 antibodies, anti-CD19 antibodies, anti-CD20 antibodies, anti-CD30 antibodies, anti-EGFR antibodies, anti-GD2 antibodies, anti-HER1 antibodies, anti-HER2 antibodies, anti-MUC1 antibodies, anti-PSMA antibodies, and anti-RQR8 antibodies.

Embodiment 167. The cell of any one of Embodiments 1-60r and 166, the method of any one of Embodiments 63-162 and 166, and the kit of any one of Embodiments 163-166, wherein the selection agent is a kill switch.

Embodiment 168. The cell of any one of Embodiments 1-60r, 166, and 167, the method of any one of Embodiments 63-162, 166, and 167, and the kit of any one of Embodiments 163-167, wherein the kill switch is selected from the group consisting of an inducible caspase 9 (iCasp9), cytosine deaminase (CDA), herpes simplex virus thymidine kinase (HSV-Tk), rapamycin-activated caspase 9 (rapaCasp9), chemically regulated-SH2-delivered inhibitory tail (CRASH-IT), nitroreductase (NTR), purine nucleoside phosphorylase (PNP), horseradish peroxidase, inducible MHC-I, inducible MHC-II, CCR4, CD16, CD19, CD20, CD30, EGFR, GD2, HER1, HER2, MUC1, PSMA, and RQR8.

Embodiment 169. The cell of any one of Embodiments 1-60r, and 166-168, the method of any one of Embodiments 63-162, 166-168, and the kit of any one of Embodiments 163-168, wherein the kill switch is iCasp9 or CDA.

Embodiment 170. The cell of any one of Embodiments 1-60r and 166, the method of any one of Embodiments 63-162 and 166, and the kit of any one of Embodiments 163-166, wherein the selection agent is degron.

Embodiment 171. The cell of any one of Embodiments 1-60r and 166-168, the method of any one of Embodiments 63-162 and 166-168, and the kit of any one of Embodiments 163-168, wherein the inducible MHC-I is selected from the group consisting of HLA-A, HLA-B, and HLA-C.

Embodiment 172. The cell of any one of Embodiments 1-60r and 166-168, the method of any one of Embodiments 63-162 and 166-168, and the kit of any one of Embodiments 163-168, wherein the inducible MHC-II is selected from the group consisting of HLA-DP, HLA-DQ, and HLA-DR.

Embodiment 173. The cell of any one of Embodiments 1-60r and 166, the method of any one of Embodiments 63-162 and 166, and the kit of any one of Embodiments 163-166, wherein the detection agent is a cell surface protein.

Embodiment 174. The cell of any one of Embodiments 1-60r, 166, and 173, the method of any one of Embodiments 63-162, 166, and 173, and the kit of any one of Embodiments 163-166 and 173, wherein the cell surface protein is selected from the group consisting of EGFR fused to a His-tag, RQRB fused to a His-tag, and CD47 fused to a His-tag.

Embodiment 175. The cell of any one of Embodiments 1-60r and 166, the method of any one of Embodiments 63-162 and 166, and the kit of any one of Embodiments 163-166, wherein the detection agent is a fluorescent protein.

Embodiment 176. The cell of any one of Embodiments 1-60r, 166, and 175, the method of any one of Embodiments 63-162, 166, and 175, and the kit of any one of Embodiments 163-166 and 175, wherein the fluorescent protein is selected from the group consisting of: green fluorescent protein (GFP), enhanced green fluorescent protein (EGFP), yellow fluorescent protein (YFP), enhanced yellow fluorescent protein (EYFP), blue fluorescent protein (BFP), enhanced blue fluorescent protein (EBFP), cyan fluorescent protein (CFP), enhanced cyan fluorescent protein (ECFP), superfolder GFP, superfolder YFP, orange fluorescent protein, red fluorescent protein, small ultrared fluorescent protein, FMN-binding fluorescent protein, dsRed, qFP611, Dronpa, TagRFP, KFP, EosFP, IrisFP, Dendra, Kaede, KikGrl, emerald fluorescent protein, Azami Green, mWasabi, TagGFP, TurboGFP, AcGFP, ZsGreen, and T-Sapphire.

Embodiment 177. The cell of any one of Embodiments 1-60r and 166, the method of any one of Embodiments 63-162 and 166, and the kit of any one of Embodiments 163-166, wherein the detection agent is a blood-detectable biomarker.

Embodiment 178. The cell of any one of Embodiments 1-60r, 166, and 173-177, the method of any one of Embodiments 63-162, 166, and 173-177, and the kit of any one of Embodiments 163-166 and 173-177, wherein the detection agent is associated with a barcode.

Embodiment 179. The cell of any one of Embodiments 1-60r, 166, and 173-178, the method of any one of Embodiments 1-60r, 166, and 173-178, and the kit of any one of Embodiments 1-60r, 166, and 173-178, wherein the barcode is integrated in a 5′ UTR or a 3′ UTR of the endogenous proliferation gene.

Embodiment 180. The cell of any one of Embodiments 1-60r, 166, and 173-178, the method of any one of Embodiments 1-60r, 166, and 173-178, and the kit of any one of Embodiments 1-60r, 166, and 173-178, wherein the barcode is integrated in a 5′ UTR or a 3′ UTR of the off-target cell marker gene.

Embodiment 181. The cell of any one of Embodiments 1-60r and 166, the method of any one of Embodiments 63-162 and 166, and the kit of any one of Embodiments 163-166, wherein the detection agent is a secreted protein.

Embodiment 182. The cell of any one of Embodiments 1-60r, 166, and 181, the method of any one of Embodiments 1-60r, 166, and 181, and the kit of any one of Embodiments 163-166 and 181, wherein the secreted protein is detectable in vitro.

Embodiment 183. The cell of any one of Embodiments 1-60r, 166, 181, and 182, the method of any one of Embodiments 63-162, 166, 181, and 182, and the kit of any one of Embodiments 163-166, 181, and 182, wherein in vitro detection comprises detecting the secreted protein in cell culture medium collected from a cell culture comprising the cell of any one of Embodiments 1-60r.

Embodiment 184. The cell of any one of Embodiments 1-60r, 166, and 181, the method of any one of Embodiments 1-60r, 166, and 181, and the kit of any one of Embodiments 163-166 and 181, wherein the secreted protein is detectable ex vivo.

Embodiment 185. The cell of any one of Embodiments 1-60r, 166, 181, and 184, the method of any one of Embodiments 63-162, 166, 181, and 184, and the kit of any one of Embodiments 163-166, 181, and 184, wherein ex vivo detection comprises detecting the secreted protein in a blood sample taken from an individual administered the cells of any one of Embodiments 1-60r.

Embodiment 186. The cell of any one of Embodiments 1-60r and 166-185, the method of any one of Embodiments 64-185, and the kit of any one of Embodiments 163-185, wherein the cell is modified to express an engineered receptor.

Embodiment 187. The cell of any one of Embodiments 1-60r and 166-186, the method of any one of Embodiments 64-186, and the kit of any one of Embodiments 163-186, wherein the engineered receptor is a chimeric antigen receptor (CAR).

Embodiment 188. The cell of any one of Embodiments 1-60r and 166-187, the method of any one of Embodiments 64-187, and the kit of any one of Embodiments 163-187, wherein the CAR comprises an extracellular antigen binding domain specifically recognizing a target antigen; a transmembrane domain; and an intracellular signaling domain.

Embodiment 189. The cell of any one of Embodiments 1-60r and 166-188, the method of any one of Embodiments 64-188, and the kit of any one of Embodiments 163-188, wherein the cell is an immune cell.

Embodiment 190. The cell of any one of Embodiments 1-60r and 166-189, the method of any one of Embodiments 64-189, and the kit of any one of Embodiments 163-189, wherein the immune cell is a T cell, a B cell, or a natural killer (NK) cell.

Embodiment 191. The cell of any one of Embodiments 1-60r and 166-190, the method of any one of Embodiments 64-190, and the kit of any one of Embodiments 163-190, wherein the cell is a mammalian cell.

Embodiment 192. The cell of any one of Embodiments 1-60r and 166-191, the method of any one of Embodiments 64-191, and the kit of any one of Embodiments 163-191, wherein the mammalian cell is a human cell.

Embodiment 193. The cell of any one of Embodiments 1-60r and 166-192, the method of any one of Embodiments 64-192, and the kit of any one of Embodiments 163-192, wherein the cell is a stem cell-derived cell.

Embodiment 194. The cell of any one of Embodiments 1-60r and 166-193, the method of any one of Embodiments 64-193, and the kit of any one of Embodiments 163-193, wherein the stem cell-derived cell is derived from a cell selected from the group consisting of embryonic stem cell, induced pluripotent stem cell, multipotent stem cell, adult stem cell, hematopoietic stem cell, mesenchymal stem cell, endothelial stem cell, epithelial stem cell, adipose stem or progenitor cells, germline stem cells, lung stem or progenitor cells, mammary stem cells, olfactory adult stem cells, hair follicle stem cells, multipotent stem cells, amniotic stem cells, cord blood stem cells, neural stem, and progenitor cells.

Embodiment 195. The cell of any one of Embodiments 1-60r and 166-194, the method of any one of Embodiments 64-194, and the kit of any one of Embodiments 163-194, wherein the stem-cell derived cell is selected from the group consisting of a stem cell-derived beta cell, an alpha cell, and a delta cell.

Embodiment 195a. The cell of any one of Embodiments 1-60r and 166-195, the method of any one of Embodiments 64-195, and the kit of any one of Embodiments 163-195, wherein the stem-cell derived cell is a stem cell-derived beta cell (SC-beta cell).

Embodiment 195b. The cell of any one of Embodiments 1-60r and 166-195a, the method of any one of Embodiments 64-195a, and the kit of any one of Embodiments 163-195a, wherein the stem-cell derived cell is a stem cell-derived beta islet cell.

Embodiment 195c. The cell of any one of Embodiments 1-60r and 166-195a, the method of any one of Embodiments 64-195a, and the kit of any one of Embodiments 163-195a, wherein the stem-cell derived cell is a stem cell-derived T cell.

Embodiment 195d. The cell of Embodiment 195c, the method of Embodiment 195c, and the kit of Embodiment 195c, wherein the stem cell-derived T cell is selected from the group consisting of an ab T cell, dg T cell, helper/regulatory T cell, cytotoxic T cell, progenitor T cell (e.g., a progenitor T cell that is CD34+CD7+CD1a- or CD34+CD7+CD5+CD1a−), naive T cell, central memory T cell, effector T cell, terminal effector T cell, immature T cell, mature T cell, natural killer T cell, naive T cell, naive central memory T cell (TCM cell), effector memory T cell (TEM cell), and effector memory RA T cell (TEMRA cell).

Embodiment 195e. The cell of any one of Embodiments 1-60r and 166-195, the method of any one of Embodiments 64-195, and the kit of any one of Embodiments 163-195, wherein the stem-cell derived cell is a stem cell-derived neural cell.

Embodiment 195f. The cell of Embodiment 195e, the method of Embodiment 195e, and the kit of Embodiment 195e, wherein the stem cell-derived neural cell is selected from the group consisting of a glial cell, cerebral endothelial cell, neuron, ependymal cell, astrocyte, microglial cell, oligodendrocyte, and a Schwann cell.

Embodiment 195g. The cell of any one of Embodiments 1-60r and 166-195, the method of any one of Embodiments 64-195, and the kit of any one of Embodiments 163-195, wherein the stem-cell derived cell is a stem cell-derived cardiac cell.

Embodiment 195h. The cell of Embodiment 195g, the method of Embodiment 195g, and the kit of Embodiment 195g, wherein the stem cell-derived cardiac cell is selected from the group consisting of cardiomyocytes, nodal cardiomyocytes, conducting cardiomyocytes, working cardiomyocytes, cardiomyocyte precursors, cardiomyocyte progenitor cell, cardiac stem cell, and cardiac muscle cells.

Embodiment 196. The cell of any one of Embodiments 1-60r and 166-195h, the method of any one of Embodiments 64-195h, and the kit of any one of Embodiments 163-195h, wherein the cell comprises modifications that (i) increase expression of one or more tolerogenic factors, and (ii) reduce expression of one or more major histocompatibility complex (MHC) class I molecules and/or one or more MHC class II molecules, wherein the increased expression of (i) and the reduced expression of (ii) is relative to a cell of the same cell type that does not comprise the modifications.

Embodiment 197. The cell of any one of Embodiments 1-60r and 166-196, the method of any one of Embodiments 64-196, and the kit of any one of Embodiments 163-196, wherein the one or more of the modifications in (ii) reduce expression of: a. one or more MHC class I molecules; b. one or more MHC class II molecules; or c. one or more MHC class I molecules and one or more MHC class II molecules.

Embodiment 198. The cell of any one of Embodiments 1-60r and 166-197, the method of any one of Embodiments 64-197, and the kit of any one of Embodiments 163-197, wherein the one or more modifications reduce expression of one or more molecules selected from the group consisting of B2M, TAP I, NLRC5, CIITA, HLA-A, HLA-B, HLA-C, HLA-DP, HLA-DM, HLA-DOA, HLA-DOB, HLA-DQ, HLA-DR, RFX5, RFXANK, RFXAP, NFY-A, NFY-B and/or NFY-C and any combination thereof.

Embodiment 199. The cell of any one of Embodiments 1-60r and 166-198, the method of any one of Embodiments 64-198, and the kit of any one of Embodiments 163-198, wherein the engineered cell does not express one or more molecules selected from the group consisting of B2M, TAP I, NLRC5, CITTA, HLA-A, HLA-B, HLA-C, HLA-DP, HLA-DM, HLA-DOA, HLA-DOB, HLA-DQ, HLA-DR, and combinations thereof.

Embodiment 200. The cell of any one of Embodiments 1-60r and 166-199, the method of any one of Embodiments 64-199, and the kit of any one of Embodiments 163-199, wherein the one or more tolerogenic factors is selected from the group consisting of CD47, A20/TNFAIP3, C1-Inhibitor, CCL21, CCL22, CD16, CD16 Fc receptor, CD24, CD27, CD35, CD39, CD46, CD52, CD55, CD59, CD200, CR1, CTLA4-Ig, DUX4, FasL, H2-M3, HLA-C, HLA-E, HLA-E heavy chain, HLA-G, PD-L1, IDO1, IL-10, IL15-RF, IL-35, MANF, Mfge8, PD-1, PD-L1 or Serpinb9, and any combination thereof.

Embodiment 201. The cell of any one of Embodiments 1-60r and 166-200, the method of any one of Embodiments 64-200, and the kit of any one of Embodiments 163-200, wherein the one or more tolerogenic factors is selected from the group consisting of: a) CD47; b) HLA-E; c) CD24; d) PD-L1; e) CD46; f) CD55; f) CD59; h) CR1; i) MANF; j) A20/TNFAIP3; k) HLA-E and CD47; 1) CD24, CD47, PD-L1, and any combination thereof; m) HLA-E, CD24, CD47, and PD-L1, and any combination thereof; n) CD46, CD55, CD59, and CR1, and any combination thereof; o) HLA-E, CD46, CD55, CD59, and CR1, and any combination thereof; p) HLA-E, CD24, CD47, PDL1, CD46, CD55, CD59, and CR1, and any combination thereof; q) HLA-E and PDL1; r) HLA-E, PDL1, and A20/TNFAIP, and any combination thereof; sHLA-E, PDL1, and MANF, and any combination thereof; t) HLA-E, PDL1, A20/TNFAIP, and MANF, and any combination thereof; and u) CD47, PD-L1, HLA-E, HLA-G, CCL21, FASL, SERPINB9, CD200, MFGE8, and any combination thereof.

Embodiment 202. The cell of any one of Embodiments 1-60r and 166-201, the method of any one of Embodiments 64-201, and the kit of any one of Embodiments 163-201, wherein the engineered cell comprises modifications according to the following: (i) (a) reduces expression of MHC I and/or MHC II; (b) reduces expression of MIC-A and/or MIC-B; (c) increases expression of CD47, and optionally CD24 and PD-L1; and (d) increases expression of CD46, CD55, CD59 and CR1; (ii) (a) reduces expression of MHC class I molecule; (b) reduces expression of MIC-A and/or MIC-B; (c) reduces expression of TXNIP; (d) increases expression of PD-L1 and HLA-E; and (e) optionally increases expression of A20/TNFAIP3 and MANF; (iii) (a) increases expression of CCL21, PD-L1, FASL, SERPINB9, HLA-G, CD47, CD200, and MFGE8; and (b) reduces expression of a MICA and/or MICB; (iv) (a) reduces expression of MHC I and/or MHC II; and (b) increases expression of CD47; or (v) any of (i)-(iv) above further comprising modifications for increasing or decreasing expression of one or more additional genes, optionally reducing expression of B2M, TAP I, NLRC5, CIITA, HLA-A, HLA-B, HLA-C, HLA-DP, HLA-DM, HLA-DOA, HLA-DOB, HLA-DQ, HLA-DR, RFX5, RFXANK, RFXAP, NFY-A, NFY-B, NFY-C, CTLA-4, PD-1, IRF1, MIC-A, MIC-B, a protein that is involved in oxidative or ER stress, TRAC, TRB, CD142, ABO, CD38, PCDH11Y, NLGN4Y and/or RHD, further optionally wherein proteins that are is involved in oxidative or ER stress include thioredoxin-interacting protein (TXNIP), PKR-like ER kinase (PERK), inositol-requiring enzyme 1α (IRE1α), and DJ-1 (PARK7).

Embodiment 203. The cell of any one of Embodiments 1-60r and 166-202, the method of any one of Embodiments 64-202, and the kit of any one of Embodiments 163-202, wherein the cell comprises modifications that (i) increase expression of one or more tolerogenic factors selected from the group consisting of CD47, PD-L1, HLA-E, HLA-G, CCL21, FASL, SERPINB9, CD200, MFGE8, and any combination thereof, and (ii) reduce expression of one or more major histocompatibility complex (MHC) class I molecules and/or one or more MHC class II molecules, wherein the increased expression of (i) and the reduced expression of (ii) is relative to a cell of the same cell type that does not comprise the modifications.

Embodiment 204. The cell of any one of Embodiments 1-60r and 166-203, the method of any one of Embodiments 64-203, and the kit of any one of Embodiments 163-203, wherein the modification(s) that increase expression comprise increased surface expression, and/or the modifications that reduce expression comprise reduced surface expression, optionally wherein there is no detectable surface expression.

Embodiment 205. The cell of any one of Embodiments 1-60r and 166-204, the method of any one of Embodiments 64-204, and the kit of any one of Embodiments 163-204, wherein the modification that increases expression of the one or more tolerogenic factors comprises an exogenous polynucleotide encoding the one or more tolerogenic factors.

Embodiment 206. The cell of any one of Embodiments 1-60r and 166-205, the method of any one of Embodiments 64-205, and the kit of any one of Embodiments 163-205, wherein the one or more tolerogenic factors comprises CD47.

Embodiment 207. The cell of Embodiment 205 or 206, the method of Embodiment 205 or 206, and the kit of Embodiment 205 or 206, wherein the one or more tolerogenic factors is CD47 and the exogenous polynucleotide encoding CD47 encodes a sequence of amino acids having at least 85% identity to the amino acid sequence of SEQ ID NO: 2, and reduces innate immune killing of the engineered primary cell.

Embodiment 208. The cell of Embodiment 207, the method of Embodiment 207, and the kit of Embodiment 207, wherein the exogenous polynucleotide encoding CD47 encodes a sequence set forth in SEQ ID NO: 2.

Embodiment 209. The cell of any one of Embodiments 1-60r and 166-208, the method of any one of Embodiments 64-208, and the kit of any one of Embodiments of any of Embodiments 163-208, wherein the exogenous polynucleotide encoding the one or more tolerogenic factors is operably linked to a promoter.

Embodiment 210. The cell of Embodiment 209, the method of Embodiment 209, and the kit of Embodiment 209, wherein the promoter is a constitutive promoter.

Embodiment 211. The cell of Embodiment 209 or 210, the method of Embodiment 209 or 210, and the kit of Embodiment 209 or 210, wherein the promoter is selected from the group consisting of the CAG promoter, the cytomegalovirus (CMV) promoter, the EF1α promoter, the PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein barr virus (EBV) promoter, the Rous sarcoma virus (RSV) promoter and the UBC promoter.

Embodiment 212. The cell of any one of Embodiments 1-60r and 166-211, the method of any one of Embodiments 64-211, and the kit of any one of Embodiments 163-211, wherein the exogenous polynucleotide encoding CD47 is integrated into the genome of the engineered primary cell.

Embodiment 213. The cell of Embodiment 212, the method of Embodiment 212, and the kit of Embodiment 212, wherein the exogenous polynucleotide is a multicistronic vector encoding the one or more tolerogenic factors and an additional transgene encoding a second transgene.

Embodiment 214. The cell of Embodiment 212 or 213, the method of Embodiment 212 or 213, and the kit of Embodiment 212 or 213, wherein the integration is by non-targeted insertion into the genome of the engineered primary cell, optionally by introduction of the exogenous polynucleotide into the cell using a lentiviral vector.

Embodiment 215. The cell of Embodiment 212 or 213, the method of Embodiment 212 or 213, and the kit of Embodiment 212 or 213, wherein the integration is by targeted insertion into a target genomic locus of the cell.

Embodiment 216. The cell of Embodiment 215, the method of Embodiment 215, and the kit of Embodiment 215, wherein the target genomic locus is a B2M gene locus, a CIITA gene locus, a MICA locus, a MICB locus, a TRAC gene locus, or a TRBC gene locus.

Embodiment 217. The cell of Embodiment 215, the method of Embodiment 215, and the kit of Embodiment 215, wherein the target genomic locus is selected from the group consisting of: a CCR5 gene locus, a CXCR4 gene locus, a PPP1R12C (also known as AAVS1) gene, an albumin gene locus, a SHS231 locus, a CLYBL gene locus, a ROSA26 gene locus, ABO gene locus, F3 gene locus, FUT1 gene locus, HMGB1 gene locus, KDM5D gene locus, LRP1 gene locus, RHD gene locus, ROSA26 gene locus, and SHS231 gene locus.

Embodiment 218. The cell of any one of Embodiments 1-60r and 166-217, the method of any one of Embodiments 64-217, and the kit of any one of Embodiments 163-217, wherein the modification that reduces expression of one or more MHC class I molecules reduces one or more MHC class I molecules protein expression.

Embodiment 219. The cell of any one of Embodiments 1-60r and 166-218, the method of any one of Embodiments 64-218, and the kit of any one of Embodiments 163-218, wherein the modification that reduces expression of one or more MHC class I molecules is a modification that reduces expression of B-2 microglobulin (B2M).

Embodiment 220. The cell of Embodiment 219, the method of Embodiment 219, or the kit of Embodiment 219, wherein the modification that reduces expression of one or more MHC class I molecules comprises reduced mRNA expression of B2M.

Embodiment 221. The cell of Embodiment 219, the method of Embodiment 219, and the kit of Embodiment 219, wherein the modification that reduces expression of one or more MHC class I molecules comprises reduced protein expression of B2M.

Embodiment 222. The cell of any one of Embodiments 1-60r and 166-221, the method of any one of Embodiments 64-221, and the kit of any one of Embodiments 163-221, wherein the modification eliminates B2M gene activity.

Embodiment 223. The cell of any one of Embodiments 1-60r and 166-222, the method of any one of Embodiments 64-222, and the kit of any one of Embodiments 163-222, wherein the modification comprises inactivation or disruption of both alleles of the B2M gene.

Embodiment 224. The cell of any one of Embodiments 1-60r and 166-223, the method of any one of Embodiments 64-223, and the kit of any one of Embodiments 163-223, wherein the modification comprises inactivation or disruption of all B2M coding sequences in the cell.

Embodiment 225. The cell of Embodiment 223 or 224, the method of Embodiment 223 or 224, and the kit of Embodiment 223 or 224, wherein the inactivation or disruption comprises an indel in the B2M gene.

Embodiment 226. The cell of any one of Embodiments 1-60r and 166-225, the method of any one of Embodiments 64-225, and the kit of any one of Embodiments 163-225, wherein the modification is a frameshift mutation or a deletion of a contiguous stretch of genomic DNA of the B2M gene.

Embodiment 227. The cell of any one of Embodiments 1-60r and 166-226, the method of any one of Embodiments 64-226, and the kit of any one of Embodiments 163-226, wherein the B2M gene is knocked out.

Embodiment 228. The cell of any one of Embodiments 218-227, the method of any one of Embodiments 218-227, and the kit of any one of Embodiments 218-227, wherein the modification that reduces expression of one or more MHC class I molecules is a modification that reduces expression of an HLA-A protein, an HLA-B protein, or HLA-C protein, optionally wherein a gene encoding said HLA-A protein, an HLA-B protein, or HLA-C protein is knocked out.

Embodiment 229. The cell of any one of Embodiments 1-60r and 166-228, the method of any one of Embodiments 64-228, and the kit of any one of Embodiments 163-228, wherein the modification that reduces expression of one or more MHC class II molecules reduces one or more MHC class II molecules protein expression.

Embodiment 230. The cell of any one of Embodiments 1-60r and 166-229, the method of any one of Embodiments 64-229, and the kit of any one of Embodiments 163-229, wherein the modification that reduces expression of one or more MHC class II molecules is a modification that reduces expression of CIITA.

Embodiment 231. The cell of Embodiment 230, the method of Embodiment 230, and the kit of Embodiment 230, wherein the modification that reduces expression of one or more MHC class II molecules comprises reduced mRNA expression of CIITA.

Embodiment 232. The cell of Embodiment 230, the method of Embodiment 230, and the kit of Embodiment 230, wherein the modification that reduces expression of one or more MHC class II molecules comprises reduced protein expression of CIITA.

Embodiment 233. The cell of any one of Embodiments 230-232, the method of any one of Embodiments 230-232, and the kit of any one of Embodiments 230-232, wherein the modification eliminates CIITA gene activity.

Embodiment 234. The cell of any one of Embodiments 230-233, the method of any one of Embodiments 230-233, and the kit of any one of Embodiments 230-233, wherein the modification comprises inactivation or disruption of both alleles of the CIITA gene.

Embodiment 235. The cell of any one of Embodiments 230-234, the method of any one of Embodiments 230-234, and the kit of any one of Embodiments 230-234, wherein the modification comprises inactivation or disruption of all CIITA coding sequences in the cell.

Embodiment 236. The cell of Embodiment 234 or 235, the method of Embodiment 234 or 235, and the kit of Embodiment 234 or 235, wherein the inactivation or disruption comprises an indel in the CIITA gene.

Embodiment 237. The cell of any one of Embodiments 230-236, the method of any one of Embodiments 230-236, and the kit of any one of Embodiments 230-236, wherein the indel is a frameshift mutation or a deletion of a contiguous stretch of genomic DNA of the CIITA gene.

Embodiment 238. The cell of any one of Embodiments 230-237, the method of any one of Embodiments 230-237, and the kit of any one of Embodiments 230-237, wherein the modification that reduces expression of one or more MHC class II molecules is a modification that reduces expression of an HLA-DP protein, an HLA-DR protein, or HLA-DQ protein, optionally wherein a gene encoding said HLA-DP protein, an HLA-DR protein, or HLA-DQ protein is knocked out.

Embodiment 239. The cell of any one of Embodiments 1-60r and 166-239, the method of any one of Embodiments 64-239, and the kit of any one of Embodiments 163-239, wherein the modification that reduces expression of the one or more MHC class I molecules and/or the one or more MHC class II molecules is by a genome-modifying protein.

Embodiment 240. The cell of Embodiment 239, the method of Embodiment 239, and the kit of Embodiment 239, wherein the genome-modifying protein is associated with gene editing by a sequence-specific nuclease, a CRISPR-associated transposase (CAST), prime editing, or Programmable Addition via Site-specific Targeting Elements (PASTE).

Embodiment 241. The cell of Embodiment 239 or 240, the method of Embodiment 239 or 240, and the kit of Embodiment 239 or 240, wherein the modification by the genome-modifying protein is nuclease-mediated gene editing.

Embodiment 242. The cell of Embodiment 241, the method of Embodiment 241, and the kit of Embodiment 241, wherein the nuclease-mediated gene editing is by a zinc finger nuclease (ZFN), a TAL-effector nuclease (TALEN), or a CRISPR-Cas combination that targets the B2M gene, optionally wherein the Cas is Cas9.

Embodiment 243. The cell of Embodiment 241 or 242, the method of Embodiment 241 or 242, and the kit of Embodiment 241 or 242, wherein the nuclease-mediated gene editing is by a CRISPR-Cas combination and the CRISPR-Cas combination comprises a guide RNA (gRNA) having a targeting domain that is complementary to at least one target site of an endogenous gene for reducing the expression of the one or more MHC class I molecules and/or one or more MHC class II molecules.

Embodiment 244. The cell of Embodiment 243, the method of Embodiment 243, and the kit of Embodiment 243, wherein the CRISPR-Cas combination is a ribonucleoprotein (RNP) complex comprising the gRNA and a Cas protein.

Embodiment 245. The cell of any one of Embodiments 1-60r and 166-244, the method of any one of Embodiments 64-244, and the kit of any one of Embodiments 163-244, wherein the engineered primary cell is a human cell or an animal cell.

Embodiment 246. The cell of Embodiment 245, the method of Embodiment 245, and the kit of Embodiment 245, wherein the engineered primary cell is a human cell.

Embodiment 247. The cell of any one of Embodiments 1-60r and 166-246, the method of any one of Embodiments 64-246, and the kit of any one of Embodiments 163-246, wherein the primary cell is a cell type that is exposed to the blood.

Embodiment 248. The cell of any one of Embodiments 1-60r and 166-247, the method of any one of Embodiments 64-247, and the kit of any one of Embodiments 163-247, wherein the engineered primary cell is a primary cell isolated from a donor subject.

Embodiment 249. The cell of any Embodiment 248, the method of Embodiment 248, and the kit of Embodiment 248, wherein the donor subject is healthy or is not suspected of having a disease or condition at the time the donor sample is obtained from the donor subject.

Embodiment 250. The cell of any one of Embodiments 1-60r and 166-249, the method of any one of Embodiments 64-249, and the kit of any one of Embodiments 163-249, wherein the engineered primary cell is selected from an islet cell, a beta islet cell, a pancreatic islet cell, an immune cell, a B cell, a T cell, a natural killer (NK) cell, a natural killer T (NKT) cell, a macrophage cell, an endothelial cell, a muscle cell, a cardiac muscle cell, a smooth muscle cell, a skeletal muscle cell, a dopaminergic neuron, a retinal pigmented epithelium cell, a hepatocyte, a thyroid cell, a skin cell, a glial progenitor cell, a neural cell, a cardiac cell, and a blood cell.

Embodiment 251. The cell of any one of Embodiments 1-60r and 166-250, the method of any one of Embodiments 64-250, and the kit of any one of Embodiments 163-250, wherein the engineered primary cell is an endothelial cell.

Embodiment 252. The cell of any one of Embodiments 1-60r and 166-250, the method of any one of Embodiments 64-250, and the kit of any one of Embodiments 163-250, wherein the engineered primary cell is an epithelial cell.

Embodiment 253. The cell of any one of Embodiments 1-60r and 166-250, the method of any one of Embodiments 64-250, and the kit of any one of Embodiments 163-250, wherein the engineered primary cell is a retinal pigmented epithelial cell.

Embodiment 254. The cell of any one of Embodiments 1-60r and 166-250, the method of any one of Embodiments 64-250, and the kit of any one of Embodiments 163-250, wherein the engineered primary cell is a T cell.

Embodiment 255. The cell of any one of Embodiments 1-60r and 166-250, the method of any one of Embodiments 64-250, and the kit of any one of Embodiments 163-250, wherein the engineered primary cell is an NK cell.

Embodiment 256. The cell of Embodiment 254 or 255, the method of Embodiment 254 or 255, and the kit of Embodiment 254 or 255, wherein the engineered primary cell comprises a chimeric antigen receptor (CAR).

Embodiment 257. The cell of any one of Embodiments 1-60r and 166-250, the method of any one of Embodiments 64-250, and the kit of any one of Embodiments 163-250, wherein the engineered primary cell is an islet cell, optionally a beta islet cell.

Embodiment 258. The cell of any one of Embodiments 1-60r and 166-250, the method of any one of Embodiments 64-250, and the kit of any one of Embodiments 163-250, wherein the engineered primary cell is a hepatocyte.

Embodiment 259. The cell of any one of Embodiments 1-60r and 166-258, the method of any one of Embodiments 64-258, and the kit of any one of Embodiments 163-258, wherein the engineered primary cell is ABO blood group type O.

Embodiment 260. The cell of any one of Embodiments 1-60r and 166-259, the method of any one of Embodiments 64-259, and the kit of any one of Embodiments 163-259, wherein the engineered primary cell is Rhesus factor negative (Rh−).

Embodiment 261. The cell of any one of Embodiments 1-60r and 166-260, the method of any one of Embodiments 64-260, and the kit of any one of Embodiments 163-260, wherein the engineered cell is capable of controlled killing of the engineered cell.

Embodiment 262. The cell of any one of Embodiments 1-60r and 166-261, the method of any of Embodiments 64-261, or the kit of any one of Embodiments 163-261, wherein the engineered cell comprises a suicide gene or a suicide switch.

Embodiment 263. The cell of Embodiment 262, the method of Embodiment 262, or the kit of Embodiment 262, wherein the suicide gene or the suicide switch induces controlled cell death in the presence of a drug or prodrug, or upon activation by a selective exogenous compound.

Embodiment 264. The cell of any one of Embodiments 1-60r and 166-263, the method of any of Embodiments 64-263 or the kit of any one of Embodiments 163-263, comprising administering an agent that allows for depletion of an engineered cell of the population of engineered cells.

Embodiment 265. The cell of any one of Embodiments 1-60r and 166-264, the method of any of Embodiments 64-264, or the kit of any one of Embodiments 163-264, comprising administering an agent that recognizes the one or more tolerogenic factors on the surface of the engineered cell.

Embodiment 266. The cell of Embodiment 265, the method of Embodiment 265, or the kit of Embodiment 265, wherein the engineered cell is engineered to express the one or more tolerogenic factors.

Embodiment 267. The cell of Embodiment 265 or 266, the method of Embodiment 265 or 266, or the kit of Embodiment 265 or 266, wherein the one or more tolerogenic factors is CD47.

Embodiment 268. The cell of any one of Embodiments 1-60r and 166-267, the method of any one of Embodiment 64-267, or the kit of any one of Embodiments 163-267, wherein expression of a detection agent acts as a signal for administration of an exogenous kill switch directed against or specific to a tolerogenic agent.

Embodiment 269. The cell of Embodiment 268, the method of Embodiment 268, or the kit of Embodiment 268, wherein the exogenous kill switch is an anti-CD47 antibody.

Embodiment 270. The cell of any one of Embodiments 1-60r and 166-269, the method of any one of Embodiments 64-269, and the kit of any one of Embodiments 163-269, wherein the cells are assayed for hypoimmunogenic phenotypes.

### Second Set of Exemplary Embodiments

Embodiment 1. A cell comprising a nucleic acid encoding a selection agent or a detection agent integrated at an endogenous proliferation gene locus and operably linked to the promoter of the endogenous proliferation gene, wherein expression of the nucleic acid encoding the selection agent or the detection agent is regulated by the promoter of the endogenous proliferation gene and/or regulatory elements of the endogenous proliferation gene.

Embodiment 2. The cell of embodiment 1, wherein the endogenous proliferation gene is selected from the group consisting of AURKB, CDK1, CDC20, RRM2, BIRC5, TOP2A, PTTG1, CCNB1, TPX2, KIF11, SPC25, CENPK, SMC4, TYMS, H2AZ1, TMSB15A, CENPF, and MKI67.

Embodiment 2a. The cell of embodiment 2, wherein the endogenous proliferation gene is selected from the group consisting of AURKB, RRM2, TOP2A, PTTG1, CCNB1, SPC25, CENPK, SMC4, TYMS, H2AZ1, TMSB15A, CENPF, and MKI67.

Embodiment 3. The cell of any one of embodiments 1-2a, wherein the nucleic acid encoding the selection agent or the detection agent is integrated in an exon or an intron of the endogenous proliferation gene.

Embodiment 4. The cell of any one of embodiments 1-3, wherein the nucleic acid encoding the selection agent or the detection agent is integrated in a 5′ UTR or a 3′ UTR of the endogenous proliferation gene.

Embodiment 5. The cell of any one of embodiments 1-4, wherein following integration, the endogenous proliferation gene locus comprises i) nucleic acid encoding the endogenous proliferation gene, ii) an internal ribosomal entry site or a self-cleaving RNA site, and iii) the nucleic acid encoding the selection agent or a detection agent.

Embodiment 6. The cell of any one of embodiments 1-5, wherein:
(i) the endogenous proliferation gene is AURKB and the selection agent is iCaspase9;
(ii) the endogenous proliferation gene is CDK1 and the selection agent is iCaspase9;
(iii) the endogenous proliferation gene is CDC20 and the selection agent is iCaspase9;
(iv) the endogenous proliferation gene is RRM2 and the selection agent is iCaspase9;
(v) the endogenous proliferation gene is BIRC5 and the selection agent is iCaspase9;
(vi) the endogenous proliferation gene is TOP2A and the selection agent is iCaspase9;
(vii) the endogenous proliferation gene is PTTG1 and the selection agent is iCaspase9;
(viii) the endogenous proliferation gene is CCNB1 and the selection agent is iCaspase9;
(ix) the endogenous proliferation gene is TPX2 and the selection agent is iCaspase9;
(x) the endogenous proliferation gene is KIF11 and the selection agent is iCaspase9;
(xi) the endogenous proliferation gene is SPC25 and the selection agent is iCaspase9;
(xii) the endogenous proliferation gene is CENPK and the selection agent is iCaspase9;
(xiii) the endogenous proliferation gene is SMC4 and the selection agent is iCaspase9;
(xiv) the endogenous proliferation gene is TYMS and the selection agent is iCaspase9;
(xv) the endogenous proliferation gene is H2AZ1 and the selection agent is iCaspase9;
(xvi) the endogenous proliferation gene is TMSB15A and the selection agent is iCaspase9;
(xvii) the endogenous proliferation gene is CENPF and the selection agent is iCaspase9;
(xviii) the endogenous proliferation gene is MKI67 and the selection agent is iCaspase9;
(xix) the endogenous proliferation gene is AURKB and the selection agent is cytosine deaminase;
(xx) the endogenous proliferation gene is CDK1 and the selection agent is cytosine deaminase;
(xxi) the endogenous proliferation gene is CDC20 and the selection agent is cytosine deaminase;
(xxii) the endogenous proliferation gene is RRM2 and the selection agent is cytosine deaminase;
(xxiii) the endogenous proliferation gene is BIRC5 and the selection agent is cytosine deaminase;
(xxiv) the endogenous proliferation gene is TOP2A and the selection agent is cytosine deaminase;
(xxv) the endogenous proliferation gene is PTTG1 and the selection agent is cytosine deaminase;
(xxvi) the endogenous proliferation gene is CCNB1 and the selection agent is cytosine deaminase;
(xxvii) the endogenous proliferation gene is TPX2 and the selection agent is cytosine deaminase;
(xxviii) the endogenous proliferation gene is KIF11 and the selection agent is cytosine deaminase;
(xxix) the endogenous proliferation gene is SPC25 and the selection agent is cytosine deaminase;
(xxx) the endogenous proliferation gene is CENPK and the selection agent is cytosine deaminase;
(xxxi) the endogenous proliferation gene is SMC4 and the selection agent is cytosine deaminase;
(xxxii) the endogenous proliferation gene is TYMS and the selection agent is cytosine deaminase;
(xxxiii) the endogenous proliferation gene is H2AZ1 and the selection agent is cytosine deaminase;
(xxxiv) the endogenous proliferation gene is TMSB15A and the selection agent is cytosine deaminase;
(xxxv) the endogenous proliferation gene is CENPF and the selection agent is cytosine deaminase; or
(xxxvi) the endogenous proliferation gene is MKI67 and the selection agent is cytosine deaminase.

Embodiment 7. The cell of any one of embodiments 1-6, wherein the selection agent or the detection agent is expressed in proliferating cells.

Embodiment 8. The cell of any one of embodiments 1-7, wherein the selection agent or the detection agent is not expressed in non-proliferating cells.

Embodiment 9. The cell of any one of embodiments 1-8, wherein the selection agent or the detection agent is expressed in partially differentiated cells.

Embodiment 9a. The cell of any one of embodiments 1-9, wherein the selection agent or the detection agent is expressed in an intermediate cell type made during differentiation.

Embodiment 10. The cell of any one of embodiments 1-9a, wherein the selection agent or the detection agent is not expressed in differentiated cells.

Embodiment 10a. The cell of any one of embodiments 1-10, wherein the selection agent or the detection agent is not expressed in a therapeutic cell.

Embodiment 11. The cell of any one of embodiments 1-10a, wherein the cell is selected from the group consisting of a a pancreatic islet cell, an alpha cell, beta cell, a gamma cell, a delta cell, an epsilon cell, a T cell, a neuron, a glial cell, a cardiomyocyte, a retinal pigmented epithelial cell, a hematopoietic progenitor cell, a natural killer cell, an endothelial cell, and a lung cell.

Embodiment 12. A cell comprising a nucleic acid encoding a selection agent or a detection agent integrated at an off-target cell type gene locus and operably linked to the promoter of the off-target cell marker gene, wherein expression of the nucleic acid encoding the selection agent or the detection agent is regulated by the promoter of the off-target cell marker gene and/or regulatory elements of the off-target cell marker gene.

Embodiment 13. The cell of embodiment 12, wherein the off-target cell marker gene is selected from the group consisting of a pluripotency cell marker gene, a tumorigenic cell marker gene, a ductal marker gene, an enterochromaffin marker gene, a neural marker gene, acinar marker gene, intestinal marker gene, endothelial marker gene, mesenchymal fibroblast marker gene, muscle marker gene, osteoblast marker gene, and stromal marker gene.

Embodiment 14. The cell of embodiment 12 or 13, wherein the off-target cell marker gene is selected from the group consisting of ANXA1, KRT19, CTSC, DSC2, ARHGAP29, KRT18, KRT8, CD9, PLK2, and KRT17.

Embodiment 15. The cell of any one of embodiments 12-14, wherein the nucleic acid encoding the selection agent or the detection agent is integrated in a 5′ UTR or a 3′ UTR of the off-target cell marker gene.

Embodiment 16. The cell of any one of embodiments 12-14, wherein the nucleic acid encoding the selection agent or the detection agent is integrated in an exon or an intron of the off-target cell marker gene.

Embodiment 16a. The cell of embodiment 16, wherein the nucleic acid encoding the selection agent or the detection agent is integrated in an exon or an intron of ANXA1.

Embodiment 16b. The cell of embodiment 16a, wherein the nucleic acid encoding the selection agent or the detection agent is integrated in an exon of ANXA1, optionally wherein the exon is exon 1, 2, 3, 4, 5, 6, 7, 8, 9, 10, 11, 12, or 13.

Embodiment 16c. The cell of embodiment 16a, wherein the nucleic acid encoding the selection agent or the detection agent is integrated in an intron of ANXA1, optionally wherein the intron is intron 1, 2, 3, 4, 5, 6, 7, 8, 9, 10, 11, or 12.

Embodiment 16d. The cell of embodiment 15, wherein the nucleic acid encoding the selection agent or the detection agent is integrated in a 5′ UTR or a 3′ UTR of ANXA1.

Embodiment 16e. The cell of embodiment 16, wherein the nucleic acid encoding the selection agent or the detection agent is integrated in an exon or an intron of KRT19.

Embodiment 16f. The cell of embodiment 16e, wherein the nucleic acid encoding the selection agent or the detection agent is integrated in an exon of KRT19, optionally wherein the exon is exon 1, 2, 3, 4, 5, or 6.

Embodiment 16g. The cell of embodiment 16e, wherein the nucleic acid encoding the selection agent or the detection agent is integrated in an intron of KRT19, optionally wherein the intron is intron 1, 2, 3, 4, or 5.

Embodiment 16 h. The cell of embodiment 15, wherein the nucleic acid encoding the selection agent or the detection agent is integrated in a 5′ UTR or a 3′ UTR of KRT19.

Embodiment 16i. The cell of embodiment 16, wherein the nucleic acid encoding the selection agent or the detection agent is integrated in an exon or an intron of CTSC.

Embodiment 16j. The cell of embodiment 16i, wherein the nucleic acid encoding the selection agent or the detection agent is integrated in an exon of CTSC, optionally wherein the exon is exon 1, 2, 3, 4, 5, 6, or 7.

Embodiment 16k. The cell of embodiment 16i, wherein the nucleic acid encoding the selection agent or the detection agent is integrated in an intron of CTSC, optionally wherein the intron is intron 1, 2, 3, 4, 5, or 6.

Embodiment 161. The cell of embodiment 15, wherein the nucleic acid encoding the selection agent or the detection agent is integrated in a 5′ UTR or a 3′ UTR of CTSC.

Embodiment 16m. The cell of embodiment 16, wherein the nucleic acid encoding the selection agent or the detection agent is integrated in an exon or an intron of DSC2.

Embodiment 16n. The cell of embodiment 16m, wherein the nucleic acid encoding the selection agent or the detection agent is integrated in an exon of DSC2, optionally wherein the exon is exon 1, 2, 3, 4, 5, 6, 7, 8, 9, 10, 11, 12, 13, 14, 15, or 16.

Embodiment 16o. The cell of embodiment 16m, wherein the nucleic acid encoding the selection agent or the detection agent is integrated in an intron of DSC2, optionally wherein the intron is intron 1, 2, 3, 4, 5, 6, 7, 8, 9, 10, 11, 12, 13, 14, or 15.

Embodiment 16p. The cell of embodiment 15, wherein the nucleic acid encoding the selection agent or the detection agent is integrated in a 5′ UTR or a 3′ UTR of DSC2.

Embodiment 16q. The cell of embodiment 16, wherein the nucleic acid encoding the selection agent or the detection agent is integrated in an exon or an intron of ARHGAP29.

Embodiment 16r. The cell of embodiment 16q, wherein the nucleic acid encoding the selection agent or the detection agent is integrated in an exon of ARHGAP29, optionally wherein the exon is exon 1, 2, 3, 4, 5, 6, 7, 8, 9, 10, 11, 12, 13, 14, 15, 16, 17, 18, 19, 20, 21, 22, or 23.

Embodiment 16s. The cell of embodiment 16q, wherein the nucleic acid encoding the selection agent or the detection agent is integrated in an intron of ARHGAP29, optionally wherein the intron is intron 1, 2, 3, 4, 5, 6, 7, 8, 9, 10, 11, 12, 13, 14, 15, 16, 17, 18, 19, 20, 21, or 22.

Embodiment 16t. The cell of embodiment 15, wherein the nucleic acid encoding the selection agent or the detection agent is integrated in a 5′ UTR or a 3′ UTR of ARHGAP29.

Embodiment 16u. The cell of embodiment 16, wherein the nucleic acid encoding the selection agent or the detection agent is integrated in an exon or an intron of KRT18.

Embodiment 16v. The cell of embodiment 16u, wherein the nucleic acid encoding the selection agent or the detection agent is integrated in an exon of KRT18, optionally wherein the exon is exon 1, 2, 3, 4, 5, 6, or 7.

Embodiment 16w. The cell of embodiment 16u, wherein the nucleic acid encoding the selection agent or the detection agent is integrated in an intron of KRT18, optionally wherein the intron is intron 1, 2, 3, 4, 5, or 6.

Embodiment 16x. The cell of embodiment 15, wherein the nucleic acid encoding the selection agent or the detection agent is integrated in a 5′ UTR or a 3′ UTR of KRT18.

Embodiment 16y. The cell of embodiment 16, wherein the nucleic acid encoding the selection agent or the detection agent is integrated in an exon or an intron of KRT8.

Embodiment 16z. The cell of embodiment 16y, wherein the nucleic acid encoding the selection agent or the detection agent is integrated in an exon of KRT8, optionally wherein the exon is exon 1, 2, 3, 4, 5, 6, 7, or 8.

Embodiment 16aa. The cell of embodiment 16y, wherein the nucleic acid encoding the selection agent or the detection agent is integrated in an intron of KRT8, optionally wherein the intron is intron 1, 2, 3, 4, 5, 6, or 7.

Embodiment 16bb. The cell of embodiment 15, wherein the nucleic acid encoding the selection agent or the detection agent is integrated in a 5′ UTR or a 3′ UTR of KRT8.

Embodiment 16cc. The cell of embodiment 16, wherein the nucleic acid encoding the selection agent or the detection agent is integrated in an exon or an intron of CD9.

Embodiment 16dd. The cell of embodiment 16cc, wherein the nucleic acid encoding the selection agent or the detection agent is integrated in an exon of CD9, optionally wherein the exon is exon 1, 2, 3, 4, 5, 6, 7, or 8.

Embodiment 16ee. The cell of embodiment 16cc, wherein the nucleic acid encoding the selection agent or the detection agent is integrated in an intron of CD9, optionally wherein the intron is intron 1, 2, 3, 4, 5, 6, or 7.

Embodiment 16ff. The cell of embodiment 15, wherein the nucleic acid encoding the selection agent or the detection agent is integrated in a 5′ UTR or a 3′ UTR of CD9.

Embodiment 16gg. The cell of embodiment 16, wherein the nucleic acid encoding the selection agent or the detection agent is integrated in an exon or an intron of PLK2.

Embodiment 16hh. The cell of embodiment 16gg, wherein the nucleic acid encoding the selection agent or the detection agent is integrated in an exon of PLK2, optionally wherein the exon is exon 1, 2, 3, 4, 5, 6, 7, 8, 9, 10, 11, 12, 13, or 14.

Embodiment 16ii. The cell of embodiment 16gg, wherein the nucleic acid encoding the selection agent or the detection agent is integrated in an intron of PLK2, optionally wherein the intron is intron 1, 2, 3, 4, 5, 6, 7, 8, 9, 10, 11, 12, or 13.

Embodiment 16jj. The cell of embodiment 15, wherein the nucleic acid encoding the selection agent or the detection agent is integrated in a 5′ UTR or a 3′ UTR of PLK2.

Embodiment 16kk. The cell of embodiment 16, wherein the nucleic acid encoding the selection agent or the detection agent is integrated in an exon or an intron of KRT17.

Embodiment 16ll. The cell of embodiment 16kk, wherein the nucleic acid encoding the selection agent or the detection agent is integrated in an exon of KRT17, optionally wherein the exon is exon 1, 2, 3, 4, 5, 6, 7, or 8.

Embodiment 16 mm. The cell of embodiment 16kk, wherein the nucleic acid encoding the selection agent or the detection agent is integrated in an intron of KRT17, optionally wherein the intron is intron 1, 2, 3, 4, 5, 6, or 7.

Embodiment 16nn. The cell of embodiment 15, wherein the nucleic acid encoding the selection agent or the detection agent is integrated in a 5′ UTR or a 3′ UTR of KRT17.

Embodiment 17. The cell of any one of embodiments 12-16nn, wherein:
(i) the off-target cell marker gene is ANXA1 and the selection agent is iCaspase9;
(ii) the off-target cell marker gene is KRT19 and the selection agent is iCaspase9;
(iii) the off-target cell marker gene is CTSC and the selection agent is iCaspase9;
(iv) the off-target cell marker gene is DSC2 and the selection agent is iCaspase9;
(v) the off-target cell marker gene is ARHGAP29 and the selection agent is iCaspase9;
(vi) the off-target cell marker gene is KRT18 and the selection agent is iCaspase9;
(vii) the off-target cell marker gene is KRT8 and the selection agent is iCaspase9;
(viii) the off-target cell marker gene is CD9 and the selection agent is iCaspase9;
(ix) the off-target cell marker gene is PLK2 and the selection agent is iCaspase9;
(x) the off-target cell marker gene is KRT17 and the selection agent is iCaspase9;
(xi) the off-target cell marker gene is ANXA1 and the selection agent is cytosine deaminase;
(xii) the off-target cell marker gene is KRT19 and the selection agent is cytosine deaminase;
(xiii) the off-target cell marker gene is CTSC and the selection agent is cytosine deaminase;
(xiv) the off-target cell marker gene is DSC2 and the selection agent is cytosine deaminase;
(xv) the off-target cell marker gene is ARHGAP29 and the selection agent is cytosine deaminase;
(xvi) the off-target cell marker gene is KRT18 and the selection agent is cytosine deaminase;
(xvii) the off-target cell marker gene is KRT8 and the selection agent is cytosine deaminase;
(xviii) the off-target cell marker gene is CD9 and the selection agent is cytosine deaminase;
(xix) the off-target cell marker gene is PLK2 and the selection agent is cytosine deaminase; or
(xx) the off-target cell marker gene is KRT17 and the selection agent is cytosine deaminase.

Embodiment 17a. The cell of any one of embodiments 12-17, wherein the selection agent or the detection agent is expressed in a cell with a high level of expression of an off-target cell marker gene.

Embodiment 17b. The cell of any one of embodiments 12-17a, wherein the selection agent or the detection agent is expressed in a pluripotent cell.

Embodiment 17c. The cell of any one of embodiments 12-17b, wherein the selection agent or the detection agent is expressed in a tumorigenic cell.

Embodiment 17d. The cell of any one of embodiments 12-17c, wherein the selection agent or the detection agent is expressed in a ductal cell.

Embodiment 17e. The cell of any one of embodiments 12-17d, wherein the selection agent or the detection agent is expressed in an enterochromaffin cell.

Embodiment 17f. The cell of any one of embodiments 12-17e, wherein the selection agent or the detection agent is expressed in a neural cell.

Embodiment 17g. The cell of any one of embodiments 12-17f, wherein the selection agent or the detection agent is expressed in partially differentiated cells.

Embodiment 17 h. The cell of any one of embodiments 12-17g, wherein the selection agent or the detection agent is not expressed in differentiated cells.

Embodiment 17i. The cell of any one of embodiments 12-17h, wherein the selection agent or the detection agent is not expressed in a cell with a low level of expression or no expression of an off-target cell marker gene.

Embodiment 17j. The cell of any one of embodiments 12-17i, wherein the selection agent or the detection agent is not expressed in a therapeutic cell.

Embodiment 18. The cell of any one of embodiments 12-17j, wherein the off-target cell marker gene is not a beta cell marker gene, a T cell marker gene, a neuronal cell marker gene, a glial cell marker gene, a cardiac cell marker gene, a retinal pigment epithelium (RPE) cell marker gene, a hematopoietic progenitor cell marker gene, a natural killer cell marker gene, an endothelial cell marker gene, or a lung cell marker gene.

Embodiment 19. The cell of any one of embodiments 1-18, wherein the cell comprises a genome editing complex.

Embodiment 19a. The cell of embodiment 19, wherein the genome editing complex is encoded by one or more transgenes.

Embodiment The cell of embodiment 19 or 19a, wherein the genome editing complex comprises a genome targeting entity and a genome modifying entity.

Embodiment 19c. The cell of embodiment 19b, wherein the genome targeting entity is a nucleic acid-guided targeting entity.

Embodiment 19d. The cell of any one of embodiments 19-19b, wherein the genome targeting entity is selected from the group consisting of: a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising a gRNA and a Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), and a Type II or Type V Cas protein, or functional portions thereof.

Embodiment 19e. The cell of any one of embodiments 19-19d, wherein the genome targeting entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, and dCas13b, or functional portions thereof.

Embodiment 19f. The cell of any one of embodiments 19-19e, wherein the genome modifying entity cleaves, deaminates, nicks, polymerizes, interrogates, integrates, cuts, unwinds, breaks, alters, methylates, demethylates, or otherwise destabilizes the target locus.

Embodiment 19g. The cell of any one of embodiments 19-19f, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.

Embodiment 19 h. The cell of any one of embodiments 19-19g, wherein the genome modifying entity is selected from the group consisting of: a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, an endonuclease-deficient-Cas protein, an enzymatically inactive Cas protein, a CRISPR-associated transposase (CAST), a Type II or Type V Cas protein, base editing, prime editing, and a Programmable Addition via Site-specific Targeting Elements (PASTE), or functional portions thereof.

Embodiment 19i. The cell of any one of embodiments 19-19h, wherein the genome modifying entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor, a target-primed reverse transcription (TPRT) editor, APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, and a transcriptional repressor, or functional portions thereof.

Embodiment 19j. The cell of any one of embodiments 19-19i, wherein the genome targeting entity and the genome modifying entity are different domains of a single polypeptide.

Embodiment 19k. The cell of any one of embodiments 19-19j, wherein the genome targeting entity and genome modifying entity are two different polypeptides that are operably linked together.

Embodiment 191. The cell of any one of embodiments 19-19i, wherein the genome targeting entity and genome modifying entity are two different polypeptides that are not linked together.

Embodiment 19m. The cell of any one of embodiments 19-191, wherein the genome editing complex comprises a guide nucleic acid having a targeting domain that is complementary to at least one target locus, optionally wherein the guide nucleic acid is a guide RNA (gRNA).

Embodiment 19n. The cell of any one of embodiments 19-19m, wherein the genome editing complex is an RNA-guided nuclease.

Embodiment 19o. The cell of any one of embodiments 19n, wherein the RNA-guided nuclease comprises a Cas nuclease and a guide RNA (CRISPR-Cas combination).

Embodiment 19p. The cell of any one of embodiments 19o, wherein the CRISPR-Cas combination is a ribonucleoprotein (RNP) complex comprising the gRNA and the Cas nuclease.

Embodiment 19q. The cell of any one of embodiment 19o or 19p, wherein the Cas nuclease is a Type II or Type V Cas protein.

Embodiment 19r. The cell of any one of embodiments 19o-19q, wherein the Cas nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7.

Embodiment 20. A composition comprising the cell of any one of the preceding embodiments.

Embodiment 21. The composition of embodiment 20, wherein the cell is an allogeneic cell.

Embodiment 21a. The composition of embodiment 21, wherein the allogeneic cell is derived from one or more donors.

Embodiment 21b. The composition of embodiment 20, wherein the cell is an autologous cell.

Embodiment 22. A method of eliminating proliferating cells comprising providing a population of cells with an inducer, wherein the population of cells express a nucleic acid encoding a selection agent integrated at an endogenous proliferation gene locus and operably linked to the promoter of the endogenous proliferation gene, and wherein providing the inducer causes elimination of proliferating cells.

Embodiment 23. The method of embodiment 22, wherein the providing is administering the inducer to an individual harboring the population of cells.

Embodiment 24. The method of embodiment 22 or 23, wherein the method further comprises administering the population of cells to a subject either before or after the inducer is provided to the subject.

Embodiment 25. The method of embodiment 24, wherein the population of cells is incubated with the inducer prior to administration to the subject.

Embodiment 26. The method of embodiment 24, wherein the population of cells are administered to a subject before the inducer is provided to the subject.

Embodiment 27. The method of embodiment 22, wherein the providing comprises incubating the population of cells in vitro.

Embodiment 28. The method of any one of embodiments 22-27, wherein the endogenous proliferation gene locus is selected from the group consisting of AURKB, CDC20, RRM2, CDK1, BIRC5, TOP2A, PTTG1, CCNB1, TPX2, KIF11, SPC25, CENPK, SMC4, TYMS, H2AZ1, TMSB15A, CENPF, and MKI67.

Embodiment 28a. The method of embodiment 28, wherein the endogenous proliferation gene locus is selected from the group consisting of AURKB, RRM2, TOP2A, PTTG1, CCNB1, SPC25, CENPK, SMC4, TYMS, H2AZ1, TMSB15A, CENPF, and MKI67.

Embodiment 29. The method of any one of embodiments 22-28a, wherein the method selectively eliminates proliferating cells, cancer cells, pluripotent cells, multipotent stem cells, progenitor cells, de-differentiated cells, undifferentiated cells, and/or partially differentiated cells.

Embodiment 30. The method of any one of embodiments 22-29, wherein the population of cells comprises proliferating cells and non-proliferating cells.

Embodiment 31. The method of any one of embodiments 22-30, wherein the differentiated cells and/or non-proliferating cells are not eliminated.

Embodiment 32. A method of eliminating a specific cell type comprising providing a population of cells with an inducer, wherein the population of cells express a nucleic acid encoding a kill switch from an off-target cell marker gene locus and operably linked to the promoter of the off-target cell marker gene, and wherein providing the inducer causes elimination of an off-target cell.

Embodiment 33. The method of embodiment 32, wherein the providing is administering the inducer to an individual harboring the population of cells.

Embodiment 34. The method of embodiment 32 or 33, wherein the method further comprises administering the population of cells to a subject either before or after the inducer is provided to the subject.

Embodiment 35. The method of embodiment 34, wherein the population of cells is incubated with the inducer prior to administration to the subject.

Embodiment 36. The method of embodiment 34, wherein the population of cells are administered to a subject before the inducer is provided to the subject.

Embodiment 37. The method of embodiment 32, wherein the providing comprises incubating the population of cells in vitro.

Embodiment 38. The method of any one of embodiments 32-37, wherein the off-target cell marker gene is selected from the group consisting of ANXA1, KRT19, CTSC, DSC2, ARHGAP29, KRT18, KRT8, CD9, PLK2, and KRT17.

Embodiment 39. The method of any one of embodiments 32-38, wherein the method selectively eliminates cancer cells, pluripotent cells, multipotent stem cells, progenitor cells, de-differentiated cells, undifferentiated cells, partially differentiated cells, and/or off-target cells.

Embodiment 40. The method of any one of embodiments 32-39, wherein the inducer does not cause elimination of a cell type other than the off-target cell.

Embodiment 40a. The method of any one of embodiments 32-40, wherein on-target cells are not eliminated.

Embodiment 40b. The method of any one of embodiments 32-40a, wherein the population of cells comprises on-target cells and off-target cells.

Embodiment 40c. The method of any one of embodiments 32-40b, wherein the population of cells comprises therapeutic cells and off-target cells.

Embodiment 40d. The method of embodiment 40c, wherein therapeutic cells are not eliminated.

Embodiment 41. The method of any one of embodiments 22-40d, wherein the population of cells are stem cell derived cells or primary cells.

Embodiment 41a. The method of embodiment 41, wherein the stem cells are pluripotent stem cells, embryonic stem cells, induced pluripotent stem cells, multipotent stem cells, or adult stem cells.

Embodiment 42. The method of any one of embodiments 22-41a, wherein the cells are autologous cells.

Embodiment 43. The method of any one of embodiments 22-41a, wherein the cells are allogeneic cells.

Embodiment 43a. The method of embodiment 43, wherein the allogeneic cells are derived from one or more donors.

Embodiment 44. The method of any one of embodiments 22-43a, wherein the inducer is administered if cell proliferation is detected or if information indicating cell proliferation is obtained.

Embodiment 45. The method of any one of embodiments 22-43a, wherein the inducer is administered if expression of proliferation markers is detected or if information indicating expression of proliferation markers is obtained.

Embodiment 46. The method of any one of embodiments 22-43a, wherein the inducer is administered if an excess number of cells is detected or if information indicating an excess number of cells is obtained.

Embodiment 47. The method of any one of embodiments 22-46, further comprising detecting proliferation using flow cytometry for a proliferation marker.

Embodiment 48. The method of any one of embodiments 22-46, further comprising detecting proliferation using single-cell RNA-sequencing for expression of proliferation markers.

Embodiment 49. A method of detecting a proliferating cell expressing a detection agent in a population of cells, wherein the population of cells comprises a nucleic acid encoding the detection agent integrated at an endogenous proliferation gene locus and operably linked to the promoter of the endogenous proliferation gene, and wherein expression of the nucleic acid encoding the detection agent is regulated by the promoter of the endogenous proliferation gene and/or regulatory elements of the endogenous proliferation gene.

Embodiment 50. A method of detecting an off-target cell expressing a detection agent in a population of cells, wherein the population of cells comprises a nucleic acid encoding the detection agent integrated at an off-target cell marker gene locus and operably linked to the promoter of the off-target cell marker gene, and wherein expression of the nucleic acid encoding the detection agent is regulated by the promoter of the off-target cell marker gene and/or regulatory elements of the off-target cell marker gene.

Embodiment 51. The method of embodiment 49 or 50, further comprising removing or eliminating the proliferating cell or the off-target cell if expression of the detection agent is identified.

Embodiment 51a. The method of any one of embodiments 49-51, further comprising treating the population of cells with an agent that recognizes the detection agent, wherein treating the population of cells with the agent that recognizes the detection agent eliminates the proliferating cell or the off-target cell or targets the proliferating cell or the off-target cell for elimination.

Embodiment 51b. The method of any one of embodiments 49-51, further comprising surgically removing the proliferating cell or the off-target cell if expression of the detection agent is identified.

Embodiment 51c. The method of embodiment 49 or 50, further comprising purifying the population of cells to select for cells not expressing the detection agent prior to administration to a patient if expression of the detection agent is identified.

Embodiment 51d. The method of embodiment 49 or 50, further comprising modifying a treatment when the proliferating cell or the off-target cell expressing the detection agent is identified.

Embodiment 51e. The method of embodiment 49 or 50, further comprising administering an additional therapy when the proliferating cell or the off-target cell expressing the detection agent is identified.

Embodiment 51f. The method of embodiment 49 or 50, further comprising administering cells that are not expressing the detection agent to a subject.

Embodiment 51g. The method of embodiment 49 or 50, wherein a treatment regimen is not modified when there are no cells expressing the detection agent identified.

Embodiment 52. A method of making a cell comprising a selection agent or a detection agent integrated at an endogenous proliferation gene locus and operably linked to the promoter of the endogenous proliferation gene, the method comprising introducing a nucleic acid encoding a selection agent or a detection agent into the cell, wherein the nucleic acid encoding the selection agent or the detection agent is integrated in the genome of the cell at the endogenous proliferation gene locus.

Embodiment 53. A method of making a cell comprising a selection agent or a detection agent integrated at an off-target cell marker gene locus and operably linked to the promoter of the off-target marker gene, the method comprising introducing a nucleic acid encoding a selection agent or a detection agent into the cell, wherein the nucleic acid encoding the selection agent or the detection agent is integrated in the genome of the cell at the off-target cell marker gene locus.

Embodiment 54. The method of embodiment 52 or 53, wherein the integration of a nucleic acid comprises a gene knock-in.

Embodiment 54a. The method of embodiment 52 or 53, wherein the integration of a nucleic acid comprises a gene knock-out.

Embodiment 55. The method of any one of embodiments 52-54a wherein the integration comprises a targeted integration method.

Embodiment 55a. The method of any one of embodiments 52-55, wherein the method comprises a genome editing complex.

Embodiment 55b. The method of embodiment 55a, wherein the genome editing complex is encoded by one or more transgenes.

Embodiment 55c. The method of embodiment 55a or 55b, wherein the genome editing complex comprises a genome targeting entity and a genome modifying entity.

Embodiment 55d. The method of embodiment 55c, wherein the genome targeting entity is a nucleic acid-guided targeting entity.

Embodiment 55e. The method of any one of embodiments 55a-55c, wherein the genome targeting entity is selected from the group consisting of: a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising a gRNA and a Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), and a Type II or Type V Cas protein, or functional portions thereof.

Embodiment 55f. The method of any one of embodiments 55a-55e, wherein the genome targeting entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, and dCas13b, or functional portions thereof.

Embodiment 55g. The method of any one of embodiments 55a-55f, wherein the genome modifying entity cleaves, deaminates, nicks, polymerizes, interrogates, integrates, cuts, unwinds, breaks, alters, methylates, demethylates, or otherwise destabilizes the target locus.

Embodiment 55h. The method of any one of embodiments 55a-55g, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.

Embodiment 55i. The method of any one of embodiments 55a-55h, wherein the genome modifying entity is selected from the group consisting of: a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, an endonuclease-deficient-Cas protein, an enzymatically inactive Cas protein, a CRISPR-associated transposase (CAST), a Type II or Type V Cas protein, base editing, prime editing, and a Programmable Addition via Site-specific Targeting Elements (PASTE), or functional portions thereof.

Embodiment 55j. The method of any one of embodiments 55a-55i, wherein the genome modifying entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor, a target-primed reverse transcription (TPRT) editor, APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, and a transcriptional repressor, or functional portions thereof.

Embodiment 55k. The method of any one of embodiments 55a55j, wherein the genome targeting entity and the genome modifying entity are different domains of a single polypeptide.

Embodiment 551. The method of any one of embodiments 55a-55k, wherein the genome targeting entity and genome modifying entity are two different polypeptides that are operably linked together.

Embodiment 55m. The method of any one of embodiments 55a-55j, wherein the genome targeting entity and genome modifying entity are two different polypeptides that are not linked together.

Embodiment 55n. The method of any one of embodiments 55a-55m, wherein the genome editing complex comprises a guide nucleic acid having a targeting domain that is complementary to at least one target locus, optionally wherein the guide nucleic acid is a guide RNA (gRNA).

Embodiment 55o. The method of any one of embodiments 55a-55n, wherein the genome editing complex is an RNA-guided nuclease.

Embodiment 55p. The method of any one of embodiments 455o, wherein the RNA-guided nuclease comprises a Cas nuclease and a guide RNA (CRISPR-Cas combination).

Embodiment 55q. The method of any one of embodiments 55p, wherein the CRISPR-Cas combination is a ribonucleoprotein (RNP) complex comprising the gRNA and the Cas nuclease.

Embodiment 55r. The method of any one of embodiment 55p and 55q, wherein the Cas nuclease is a Type II or Type V Cas protein.

Embodiment 55s. The method of any one of embodiments 55p-55r, wherein the Cas nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7.

Embodiment 56. The method of embodiment 55, wherein the targeted integration method is a Cas-directed homology-directed repair (HDR).

Embodiment 57. A method of treating a patient in need thereof comprising,
(a) administering a population of cells to the patient, wherein the population of cells comprise a nucleic acid encoding a selection agent integrated at an endogenous proliferation gene locus and operably linked to the promoter of the endogenous proliferation gene, and
(b) activating the selection agent to eliminate one or more cells in the population of cells.

Embodiment 57a. The method of embodiment 57, wherein the step of activating the selection agent takes place before the population of cells are administered to the patient and/or after the population of cells are administered to the patient.

Embodiment 57b. The method of embodiment 57 or 57a, wherein the step of activating the selection agent takes place before the population of cells are administered to the patient.

Embodiment 57c. The method of embodiment 57 or 57a, wherein the step of activating the selection agent takes place after the population of cells are administered to the patient.

Embodiment 57d. The method of embodiment 57 or 57a, wherein the step of activating the selection agent takes place before the population of cells are administered to the patient and after the population of cells are administered to the patient.

Embodiment 57e. A method of treating a patient in need thereof comprising activating a selection agent to eliminate one or more cells in a population of cells, wherein the patient was previously administered the population of cells, wherein the population of cells comprise a nucleic acid encoding a selection agent integrated at an endogenous proliferation gene locus and operably linked to the promoter of the endogenous proliferation gene.

Embodiment 58. A method of treating a disease in an individual, comprising administering a cell therapy to treat the disease, wherein the cell therapy comprises a nucleic acid encoding a selection agent or a detection agent integrated at an endogenous proliferation gene locus and operably linked to the promoter of the endogenous proliferation gene.

Embodiment 59. A method of treating a disease in an individual, comprising administering a cell therapy to treat the disease, wherein the cell therapy comprises a nucleic acid encoding a selection agent or a detection agent integrated at an off-target cell marker gene locus and operably linked to the promoter of the off-target cell marker gene.

Embodiment 60. The method of embodiment 58 or 59, wherein the disease is a cellular deficiency.

Embodiment 61. The method of embodiment 60, wherein the disease is associated with diabetes or is diabetes.

Embodiment 62. The method of embodiment 61, wherein the diabetes is Type I diabetes.

Embodiment 63. The method of embodiment 58 or 59, wherein the disease is a cancer, a neurological disease, or an autoimmune disease.

Embodiment 64. The method of embodiment 63, wherein the cancer is selected from the group consisting of ovarian cancer, gastric adenocarcinoma, pancreatic adenocarcinoma, glioblastoma, hepatocellular carcinoma, B-cell chronic lymphocytic leukemia (B-CLL), juvenile chronic myelogenous leukemia (CML), juvenile myelomonocytic leukemia (JMML), Acute Lymphoblastic Leukemia (ALL), Acute Myeloid Leukemia (AML), Adrenocortical Carcinoma, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Atypical Teratoid/Rhabdoid Tumor, Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors, Burkitt Lymphoma, Carcinoid Tumor, Carcinoma, Cardiac Tumors, Atypical Teratoid/Rhabdoid Tumor, Medulloblastoma, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Endometrial Cancer, Ependymoma, Esophageal Cancer, Esthesioneuroblastoma, Ewing Sarcoma, Extracranial Germ Cell Tumor, Extragonadal Germ Cell Tumor, Intraocular Melanoma, Retinoblastoma, Fallopian Tube Cancer, Fibrous Histiocytoma of Bone, Osteosarcoma, Gallbladder Cancer, Gastric Cancer, Gastrointestinal Carcinoid Tumor, Gastrointestinal Stromal Tumors (GIST) (Soft Tissue Sarcoma), Germ Cell Tumors, Central Nervous System Germ Cell Tumors, Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Hepatocellular Cancer, Histiocytosis (Langerhans Cell), Hodgkin Lymphoma, Hypopharyngeal Cancer, Islet Cell Tumors, Pancreatic Neuroendocrine Tumors, Kaposi Sarcoma (Soft Tissue Sarcoma), Renal Cell Cancer, Langerhans Cell Histiocytosis, Laryngeal Cancer, Leukemia, Lip and Oral Cavity Cancer, Liver Cancer, Lung Cancer (Non-Small Cell, Small Cell, Pleuropulmonary Blastoma, and Tracheobronchial Tumor), Lung Squamous Cell Carcinoma, Lymphoma, Male Breast Cancer, Malignant Fibrous Histiocytoma of Bone and Osteosarcoma, Merkel Cell Carcinoma, Mesothelioma, Metastatic Cancer, Metastatic Squamous Neck Cancer with Occult Primary, Midline Tract Carcinoma With NUT Gene Changes, Oropharyngeal Cancer, Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Chronic Myeloproliferative Neoplasms, acute B lymphoblastic leukemia (B-ALL), large B cell lymphoma (LBCL), diffuse large B cell lymphoma (DLBCL), high-grade B cell lymphoma (HGBCL), primary mediastinal B cell lymphoma (PMBCL), mantle cell lymphoma (MCL), marginal zone lymphoma (MZL), or small lymphocytic lymphoma (SLL) Follicular Lymphoma, Blastic Plasmacytoid Dendritic Cell Neoplasm (BPDCN), Systemic Mastocytosis, Nasal Cavity and Paranasal Sinus Cancer, Nasopharyngeal Cancer, Neuroblastoma, Non-Hodgkin Lymphoma, Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Primary Peritoneal Cancer, Prostate Cancer, Recurrent Cancer, Rhabdomyosarcoma, Salivary Gland Cancer, Vascular Tumors, Small Intestine Cancer, Soft Tissue Sarcoma, T-Cell Lymphoma, Thymoma and Thymic Carcinoma, Transitional Cell Cancer of the Renal Pelvis and Ureter, Vaginal Cancer, Vulvar Cancer, and Wilms Tumor.

Embodiment 65. The method of embodiment 47, wherein the autoimmune disease is selected from the group consisting of arthritis, rheumatoid arthritis, acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, juvenile-onset rheumatoid arthritis, osteoarthritis, arthritis chronica progrediente, arthritis deformans, polyarthritis chronica primaria, reactive arthritis, ankylosing spondylitis, inflammatory hyperproliferative skin diseases, psoriasis, plaque psoriasis, gutatte psoriasis, pustular psoriasis, psoriasis of the nails, atopy, atopic diseases, hay fever, Job's syndrome, dermatitis, contact dermatitis, chronic contact dermatitis, exfoliative dermatitis, exfoliative psoriatic dermatitis, allergic dermatitis, allergic contact dermatitis, dermatitis herpetiformis, nummular dermatitis, seborrheic dermatitis, non-specific dermatitis, primary irritant contact dermatitis, atopic dermatitis, x-linked hyper IgM syndrome, allergic intraocular inflammatory diseases, urticaria, chronic allergic urticaria, chronic idiopathic urticaria, chronic autoimmune urticaria, myositis, polymyositis/dermatomyositis, juvenile dermatomyositis, toxic epidermal necrolysis, scleroderma, systemic scleroderma, sclerosis, systemic sclerosis, multiple sclerosis (MS), MS associated with EBV infection, spino-optical MS, primary progressive MS (PPMS), relapsing-remitting MS (RRMS), progressive relapsing MS, secondary progressive MS (SPMS), progressive systemic sclerosis, atherosclerosis, arteriosclerosis, sclerosis disseminata, ataxic sclerosis, neuromyelitis optica spectrum disorder, inflammatory bowel disease (IBD), Crohn's disease, autoimmune-mediated gastrointestinal diseases, colitis, ulcerative colitis, colitis ulcerosa, microscopic colitis, collagenous colitis, colitis polyposa, necrotizing enterocolitis, transmural colitis, autoimmune inflammatory bowel disease, bowel inflammation, pyoderma gangrenosum, erythema nodosum, primary sclerosing cholangitis, respiratory distress syndrome, adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage, meningitis, herpes gestationis, pemphigoid gestationis, pruritis scroti, autoimmune premature ovarian failure, sudden hearing loss due to an autoimmune condition, IgE-mediated diseases, anaphylaxis, allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN) with or without nephrotic syndrome, chronic or acute glomerulonephritis, primary GN, immune-mediated GN, membranous GN (membranous nephropathy), idiopathic membranous GN or idiopathic membranous nephropathy, membrano- or membranous proliferative GN (MPGN), Type I or Type II GN, rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, erythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema, allergic or atopic eczema, asteatotic eczema, dyshidrotic eczema, vesicular palmoplantar eczema, asthma, asthma bronchiale, bronchial asthma, auto-immune asthma, conditions involving infiltration of T cells or chronic inflammatory responses, immune reactions against foreign antigens, immune reactions against fetal A-B-O blood groups during pregnancy, chronic pulmonary inflammatory disease, autoimmune myocarditis, leukocyte adhesion deficiency, lupus, lupus nephritis, lupus cerebritis, pediatric lupus, non-renal lupus, extra-renal lupus, discoid lupus, discoid lupus erythematosus, alopecia lupus, systemic lupus erythematosus (SLE), cutaneous SLE, subacute cutaneous SLE, neonatal lupus syndrome (NLE), lupus erythematosus disseminatus, CNS lupus, anti-neutrophilic cytoplasmic autoantibody (ANCA) associated vasculitis, granulomatous polyangiitis, microscopic polyangiitis, autoimmune blistering skin diseases, anti-NMDA receptor neuropathy, stiff persons disease, anti-NMDA receptor encephalitis, anti-synthetase autoimmune syndromes, rapidly progressive glomerulopathy, Type I diabetes, Type II diabetes, latent autoimmune diabetes in adults, Type 1.5 diabetes, juvenile onset (Type I) diabetes mellitus, pediatric insulin-dependent diabetes mellitus (IDDM), adult onset diabetes mellitus (Type II diabetes), idiopathic diabetes, insipidus, diabetic retinopathy, diabetic nephropathy, diabetic large-artery disorder, immune responses associated with acute or delayed hypersensitivity mediated by cytokines or T-lymphocytes, tuberculosis, sarcoidosis, granulomatosis, lymphomatoid granulomatosis, Wegener's granulomatosis, agranulocytosis, vasculitides, vasculitis, large-vessel vasculitis, polymyalgia rheumatica, giant cell (Takayasu's) arteritis, medium-vessel vasculitis, Kawasaki's disease, polyarteritis nodosa/periarteritis nodosa, microscopic polyarteritis, immunovasculitis, CNS vasculitis, cutaneous vasculitis, hypersensitivity vasculitis, necrotizing vasculitis, systemic necrotizing vasculitis, ANCA-associated vasculitis, Churg-Strauss vasculitis, syndrome (CSS), ANCA-associated small-vessel vasculiti, temporal arteritis, aplastic anemia, autoimmune aplastic anemia, Coombs positive anemia, Diamond Blackfan anemia, hemolytic anemia, immune hemolytic anemia, autoimmune hemolytic anemia (AIHA), pernicious anemia (anemia perniciosa), Addison's disease, pure red cell anemia, aplasia (PRCA), Factor VIII deficiency, hemophilia A, autoimmune neutropenia, pancytopenia, leukopenia, diseases involving leukocyte diapedesis, CNS inflammatory disorders, Alzheimer's disease, Parkinson's disease, multiple organ injury syndrome, multiple organ injury syndrome secondary to septicemia, trauma, or hemorrhage, antigen-antibody complex-mediated diseases, anti-glomerular basement membrane disease, anti-phospholipid antibody syndrome, anti-phospholipid syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid, pemphigoid bullous, skin pemphigoid, pemphigus, pemphigus vulgaris, pemphigus foliaceus, pemphigus mucus-membrane pemphigoid, pemphigus erythematosus, autoimmune polyendocrinopathies, Reiter's disease or syndrome, thermal injury, preeclampsia, an immune complex disorder, immune complex nephritis, antibody-mediated nephritis, polyneuropathies, chronic neuropathy, IgM polyneuropathies, IgM-mediated neuropathy, thrombocytopenia, thrombotic thrombocytopenic purpura (TTP), post-transfusion purpura (PTP), heparin-induced thrombocytopenia, autoimmune or immune-mediated thrombocytopenia, idiopathic thrombocytopenic purpura (ITP), chronic or acute ITP, acquired thrombocytopenic purpura, scleritis, idiopathic cerato-scleritis, episcleritis, autoimmune disease of the testis or ovary, autoimmune orchitis or oophoritis, primary hypothyroidism, hypoparathyroidism, autoimmune endocrine diseases, thyroiditis, autoimmune thyroiditis, Hashimoto's disease, chronic thyroiditis, Hashimoto's thyroiditis, subacute thyroiditis, autoimmune thyroid disease, idiopathic hypothyroidism, Grave's disease, polyglandular syndromes, autoimmune polyglandular syndromes, polyglandular endocrinopathy syndromes, paraneoplastic syndromes, neurologic paraneoplastic syndromes, Lambert-Eaton myasthenic syndrome, Eaton-Lambert syndrome, stiff-man or stiff-person syndrome, encephalomyelitis, allergic encephalomyelitis, encephalomyelitis allergica, experimental allergic encephalomyelitis (EAE), myasthenia gravis, thymoma-associated myasthenia gravis, cerebellar degeneration, neuromyotonia, opsoclonus, opsoclonus myoclonus syndrome (OMS), sensory neuropathy, multifocal motor neuropathy, Sheehan's syndrome, hepatitis, autoimmune hepatitis, chronic hepatitis, lupoid hepatitis, giant cell hepatitis, chronic active hepatitis, autoimmune chronic active hepatitis, lymphoid interstitial pneumonitis (LIP), bronchiolitis obliterans, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis, primary biliary cirrhosis, pneumonocirrhosis, autoimmune enteropathy syndrome, Celiac or Coeliac disease, celiac sprue (gluten enteropathy), refractory sprue, idiopathic sprue, cryoglobulinemia, amyotrophic lateral sclerosis (ALS; Lou Gehrig's disease), coronary artery disease, autoimmune ear disease, autoimmune inner ear disease (AIED), autoimmune hearing loss, polychondritis, refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, monoclonal B cell lymphocytosis, benign monoclonal gammopathy or monoclonal gammopathy of undetermined significance, peripheral neuropathy, paraneoplastic syndrome, channelopathies, epilepsy, migraine, arrhythmia, muscular disorders, deafness, blindness, periodic paralysis, channelopathies of the CNS, autism, inflammatory myopathy, focal or segmental or focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases, autoimmune demyelinating diseases, chronic inflammatory demyelinating polyneuropathy, Dressler's syndrome, alopecia areata, alopecia totalis, CREST syndrome, calcinosis, Raynaud's phenomenon, esophageal dysmotility, sclerodactyly, telangiectasia, male or female autoimmune infertility, anti-spermatozoan antibodies, mixed connective tissue disease, Chagas' disease, rheumatic fever, recurrent abortion, farmer's lung, erythema multiforme, post-cardiotomy syndrome, post myocardial infarction cardiotomy syndrome, Cushing's syndrome, bird-fancier's lung, allergic granulomatous angiitis, benign lymphocytic angiitis, Alport's syndrome, alveolitis, allergic alveolitis, fibrosing alveolitis, interstitial lung disease, transfusion reaction, leprosy, malaria, parasitic diseases, leishmaniasis, kypanosomiasis, schistosomiasis, ascariasis, aspergillosis, Samter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis, chronic cyclitis, heterochronic cyclitis, iridocyclitis (acute or chronic), Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, non-malignant thymoma, vitiligo, toxic-shock syndrome, food poisoning, conditions involving infiltration of T cells, leukocyte-adhesion deficiency, immune responses associated with acute or delayed hypersensitivity mediated by cytokines or T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, sphenoid sinusitis, an eosinophil-related disorder, eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis, central nervous system inflammatory disorders, ocular or orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, emphysema, alopecia areata, adipose tissue inflammation/diabetes type II, obesity-associated adipose tissue inflammation/insulin resistance, endometriosis, and pulmonary hemosiderosis.

Embodiment 66. The method of embodiment 58 or 59, wherein the disease is selected from the group consisting of Parkinson's disease, Huntington disease, Alzheimer's disease, multiple sclerosis, Amyotrophic lateral sclerosis (ALS), spinocerebellar ataxia, dementia, Charcot-Marie-Tooth disease, prion disease, muscular dystrophy, other neurodegenerative disease or condition, attention deficit hyperactivity disorder (ADHD), Tourette Syndrome (TS), schizophrenia, psychosis, depression, bipolar disorder, anxiety disorder, autism spectrum disorder, other neuropsychiatric disorder, and stroke.

Embodiment 67. The method of embodiment 58 or 59, wherein the disease is a cardiac disease.

Embodiment 68. The method of embodiments 67, wherein the cardiac disorder is selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, idiopathic cardiomyopathy, other cardiomyopathy, myocardial ischemic reperfusion injury, ventricular dysfunction, heart failure, congestive heart failure, coronary artery disease, end-stage heart disease, atherosclerosis, ischemia, hypertension, restenosis, angina pectoris, rheumatic heart, arterial inflammation, cardiovascular disease, myocardial infarction, myocardial ischemia, congestive heart failure, myocardial infarction, cardiac ischemia, cardiac injury, myocardial ischemia, vascular disease, acquired heart disease, congenital heart disease, atherosclerosis, coronary artery disease, dysfunctional conduction systems, dysfunctional coronary arteries, pulmonary hypertension, cardiac arrhythmias, muscular dystrophy, muscle mass abnormality, muscle degeneration, myocarditis, infective myocarditis, drug- or toxin-induced muscle abnormalities, hypersensitivity myocarditis, and autoimmune endocarditis.

Embodiment 69. The method of any one of embodiments 49, 51a-52, 54-58, and 60-68, wherein the endogenous proliferation gene is selected from the group consisting of AURKB, CDK1, CDC20, RRM2, BIRC5, TOP2A, PTTG1, CCNB1, TPX2, KIF11, SPC25, CENPK, SMC4, TYMS, H2AZ1, TMSB15A, CENPF, and MKI67.

Embodiment 69a. The method of embodiment 69, wherein the endogenous proliferation gene is selected from the group consisting of AURKB, RRM2, TOP2A, PTTG1, CCNB1, SPC25, CENPK, SMC4, TYMS, H2AZ1, TMSB15A, CENPF, and MKI67.

Embodiment 70. The method of any one of embodiments 49, 51a-52, 54-58, and 60-69, wherein the nucleic acid encoding the selection agent or the detection agent is integrated in the exon or intron of the endogenous proliferation gene.

Embodiment 71. The method of any one of embodiments 49, 51a-52, 54-58, and 60-69, and 70, wherein the nucleic acid encoding the selection agent or the detection agent is integrated in the 5′ UTR or the 3′ UTR of the endogenous proliferation gene.

Embodiment 72. The method of any one of embodiments 49, 51a-52, 54-58, and 60-69, 70, and 71, wherein expression of the nucleic acid encoding the selection agent or a detection agent is regulated by the endogenous promoter and/or regulatory elements of the endogenous proliferation gene.

Embodiment 73. The method of any one of embodiments 49, 51a-52, 54-58, and 60-69, and 70-72, wherein following integration, the endogenous proliferation gene locus comprises i) nucleic acid encoding the proliferation gene, ii) an internal ribosomal entry site or a self-cleaving RNA site, and iii) the nucleic acid encoding the selection agent or a detection agent.

Embodiment 74. The method of any one of embodiments 49, 51a-52, 54-58, and 60-69, and 70-73, wherein:
(i) the endogenous proliferation gene is AURKB and the selection agent is iCaspase9;
(ii) the endogenous proliferation gene is CDK1 and the selection agent is iCaspase9;
(iii) the endogenous proliferation gene is CDC20 and the selection agent is iCaspase9;
(iv) the endogenous proliferation gene is RRM2 and the selection agent is iCaspase9;
(v) the endogenous proliferation gene is BIRC5 and the selection agent is iCaspase9;
(vi) the endogenous proliferation gene is TOP2A and the selection agent is iCaspase9;
(vii) the endogenous proliferation gene is PTTG1 and the selection agent is iCaspase9;
(viii) the endogenous proliferation gene is CCNB1 and the selection agent is iCaspase9;
(ix) the endogenous proliferation gene is TPX2 and the selection agent is iCaspase9;
(x) the endogenous proliferation gene is KIF11 and the selection agent is iCaspase9;
(xi) the endogenous proliferation gene is SPC25 and the selection agent is iCaspase9;
(xii) the endogenous proliferation gene is CENPK and the selection agent is iCaspase9;
(xiii) the endogenous proliferation gene is SMC4 and the selection agent is iCaspase9;
(xiv) the endogenous proliferation gene is TYMS and the selection agent is iCaspase9;
(xv) the endogenous proliferation gene is H2AZ1 and the selection agent is iCaspase9;
(xvi) the endogenous proliferation gene is TMSB15A and the selection agent is iCaspase9;
(xvii) the endogenous proliferation gene is CENPF and the selection agent is iCaspase9;
(xviii) the endogenous proliferation gene is MKI67 and the selection agent is iCaspase9;
(xix) the endogenous proliferation gene is AURKB and the selection agent is cytosine deaminase;
(xx) the endogenous proliferation gene is CDK1 and the selection agent is cytosine deaminase;
(xxi) the endogenous proliferation gene is CDC20 and the selection agent is cytosine deaminase;
(xxii) the endogenous proliferation gene is RRM2 and the selection agent is cytosine deaminase;
(xxiii) the endogenous proliferation gene is BIRC5 and the selection agent is cytosine deaminase;
(xxiv) the endogenous proliferation gene is TOP2A and the selection agent is cytosine deaminase;
(xxv) the endogenous proliferation gene is PTTG1 and the selection agent is cytosine deaminase;
(xxvi) the endogenous proliferation gene is CCNB1 and the selection agent is cytosine deaminase;
(xxvii) the endogenous proliferation gene is TPX2 and the selection agent is cytosine deaminase;
(xxviii) the endogenous proliferation gene is KIF11 and the selection agent is cytosine deaminase;
(xxix) the endogenous proliferation gene is SPC25 and the selection agent is cytosine deaminase;
(xxx) the endogenous proliferation gene is CENPK and the selection agent is cytosine deaminase;
(xxxi) the endogenous proliferation gene is SMC4 and the selection agent is cytosine deaminase;
(xxxii) the endogenous proliferation gene is TYMS and the selection agent is cytosine deaminase;
(xxxiii) the endogenous proliferation gene is H2AZ1 and the selection agent is cytosine deaminase;
(xxxiv) the endogenous proliferation gene is TMSB15A and the selection agent is cytosine deaminase;
(xxxv) the endogenous proliferation gene is CENPF and the selection agent is cytosine deaminase; or
(xxxvi) the endogenous proliferation gene is MKI67 and the selection agent is cytosine deaminase.

Embodiment 75. The method of any one of embodiments 49, 51a-52, 54-58, and 60-69, and 70-74, wherein the selection agent or the detection agent is expressed in proliferating cells.

Embodiment 76. The method of any one of embodiments 49, 51a-52, 54-58, and 60-69, and 70-75, wherein the selection agent or the detection agent is not expressed in non-proliferating cells.

Embodiment 77. The method of any one of embodiments 49, 51a-52, 54-58, and 60-69, and 70-76, wherein the selection agent or the detection agent is expressed in partially differentiated cells.

Embodiment 77a. The method of any one of embodiments 49, 51a-52, 54-58, and 60-69, and 70-77, wherein the selection agent or the detection agent is expressed in an intermediate cell type made during differentiation.

Embodiment 78. The method of any one of embodiments 49, 51a-52, 54-58, and 60-69, and 70-77a, wherein the selection agent or the detection agent is not expressed in differentiated cells.

Embodiment 78a. The method of any one of embodiments 49, 51a-52, 54-58, and 60-69, and 70-78, wherein the selection agent or the detection agent is not expressed in a therapeutic cell.

Embodiment 79. The method of any one of embodiments 49, 51a-52, 54-58, and 60-69, and 70-78a, wherein the cell is selected from the group consisting of a pancreatic islet cell, an alpha cell, a beta cell, a gamma cell, a delta cell, an epsilon cell, a T cell, a neuron, a glial cell, a cardiomyocyte, a retinal pigmented epithelial cell, a hematopoietic progenitor cell, a natural killer cell, an endothelial cell, and a lung cell.

Embodiment 80. The method of any one of embodiments 50-51g, 53-56, and 59-68, wherein the off-target cell marker gene is selected from the group consisting of a pluripotency cell marker gene, a tumorigenic cell marker gene, a ductal marker gene, an enterochromaffin marker gene, a neural marker gene, acinar marker gene, intestinal marker gene, endothelial marker gene, mesenchymal fibroblast marker gene, muscle marker gene, osteoblast marker gene, and stromal marker gene.

Embodiment 81. The method of any one of embodiments 50-51g, 53-56, 59-68, and 80, wherein the off-target cell marker gene is selected from the group consisting of ANXA1, KRT19, CTSC, DSC2, ARHGAP29, KRT18, KRT8, CD9, PLK2, and KRT17.

Embodiment 82. The method of any one of embodiments 50-51g, 53-56, 59-68, 80, and 81, wherein the nucleic acid encoding the selection agent or the detection agent is integrated in the 5′ UTR or 3′ UTR of the off-target cell marker gene.

Embodiment 83. The method of any one of embodiments 50-51g, 53-56, 59-68, and 80-82, wherein the nucleic acid encoding the selection agent or the detection agent is integrated in the exon or intron of the off-target cell marker gene.

Embodiment 83a. The method of any one of embodiments 50-51g, 53-56, 59-68, and 80-83, wherein the nucleic acid encoding the selection agent or the detection agent is integrated in an exon or an intron of ANXA1.

Embodiment 83b. The method of embodiment 83a, wherein the nucleic acid encoding the selection agent or the detection agent is integrated in an exon of ANXA1, optionally wherein the exon is exon 1, 2, 3, 4, 5, 6, 7, 8, 9, 10, 11, 12, or 13.

Embodiment 83c. The method of embodiment 83a, wherein the nucleic acid encoding the selection agent or the detection agent is integrated in an intron of ANXA1, optionally wherein the intron is intron 1, 2, 3, 4, 5, 6, 7, 8, 9, 10, 11, or 12.

Embodiment 83d. The method of any one of embodiments 50-51g, 53-56, 59-68, 80, and 81, wherein the nucleic acid encoding the selection agent or the detection agent is integrated in a 5′ UTR or a 3′ UTR of ANXA1.

Embodiment 83e. The method of any one of embodiments 50-51g, 53-56, 59-68, and 80-83, wherein the nucleic acid encoding the selection agent or the detection agent is integrated in an exon or an intron of KRT19.

Embodiment 83f. The method of embodiment 83e, wherein the nucleic acid encoding the selection agent or the detection agent is integrated in an exon of KRT19, optionally wherein the exon is exon 1, 2, 3, 4, 5, or 6.

Embodiment 83g. The method of embodiment 83e, wherein the nucleic acid encoding the selection agent or the detection agent is integrated in an intron of KRT19, optionally wherein the intron is intron 1, 2, 3, 4, or 5.

Embodiment 83h. The method of embodiment 82, wherein the nucleic acid encoding the selection agent or the detection agent is integrated in a 5′ UTR or a 3′ UTR of KRT19.

Embodiment 83i. The method of any one of embodiments 50-51g, 53-56, 59-68, and 80-83, wherein the nucleic acid encoding the selection agent or the detection agent is integrated in an exon or an intron of CTSC.

Embodiment 83j. The method of embodiment 83i, wherein the nucleic acid encoding the selection agent or the detection agent is integrated in an exon of CTSC, optionally wherein the exon is exon 1, 2, 3, 4, 5, 6, or 7.

Embodiment 83k. The method of embodiment 83i, wherein the nucleic acid encoding the selection agent or the detection agent is integrated in an intron of CTSC, optionally wherein the intron is intron 1, 2, 3, 4, 5, or 6.

Embodiment 831. The method of embodiment 82, wherein the nucleic acid encoding the selection agent or the detection agent is integrated in a 5′ UTR or a 3′ UTR of CTSC.

Embodiment 83m. The method of any one of embodiments 50-51g, 53-56, 59-68, and 80-83, wherein the nucleic acid encoding the selection agent or the detection agent is integrated in an exon or an intron of DSC2.

Embodiment 83n. The method of embodiment 83m, wherein the nucleic acid encoding the selection agent or the detection agent is integrated in an exon of DSC2, optionally wherein the exon is exon 1, 2, 3, 4, 5, 6, 7, 8, 9, 10, 11, 12, 13, 14, 15, or 16.

Embodiment 83o. The method of embodiment 83m, wherein the nucleic acid encoding the selection agent or the detection agent is integrated in an intron of DSC2, optionally wherein the intron is intron 1, 2, 3, 4, 5, 6, 7, 8, 9, 10, 11, 12, 13, 14, or 15.

Embodiment 83p. The method of embodiment 82, wherein the nucleic acid encoding the selection agent or the detection agent is integrated in a 5′ UTR or a 3′ UTR of DSC2.

Embodiment 83q. The method of any one of embodiments 50-51g, 53-56, 59-68, and 80-83, wherein the nucleic acid encoding the selection agent or the detection agent is integrated in an exon or an intron of ARHGAP29.

Embodiment 83r. The method of embodiment 83q, wherein the nucleic acid encoding the selection agent or the detection agent is integrated in an exon of ARHGAP29, optionally wherein the exon is exon 1, 2, 3, 4, 5, 6, 7, 8, 9, 10, 11, 12, 13, 14, 15, 16, 17, 18, 19, 20, 21, 22, or 23.

Embodiment 83s. The method of embodiment 83q, wherein the nucleic acid encoding the selection agent or the detection agent is integrated in an intron of ARHGAP29, optionally wherein the intron is intron 1, 2, 3, 4, 5, 6, 7, 8, 9, 10, 11, 12, 13, 14, 15, 16, 17, 18, 19, 20, 21, or 22.

Embodiment 83t. The method of embodiment 82, wherein the nucleic acid encoding the selection agent or the detection agent is integrated in a 5′ UTR or a 3′ UTR of ARHGAP29.

Embodiment 83u. The method of any one of embodiments 50-51g, 53-56, 59-68, and 80-83, wherein the nucleic acid encoding the selection agent or the detection agent is integrated in an exon or an intron of KRT18.

Embodiment 83v. The method of embodiment 83u, wherein the nucleic acid encoding the selection agent or the detection agent is integrated in an exon of KRT18, optionally wherein the exon is exon 1, 2, 3, 4, 5, 6, or 7.

Embodiment 83w. The method of any one of embodiment 83u, wherein the nucleic acid encoding the selection agent or the detection agent is integrated in an intron of KRT18, optionally wherein the intron is intron 1, 2, 3, 4, 5, or 6.

Embodiment 83x. The method of embodiment 82, wherein the nucleic acid encoding the selection agent or the detection agent is integrated in a 5′ UTR or a 3′ UTR of KRT18.

Embodiment 83y. The method of any one of embodiments 50-51g, 53-56, 59-68, and 80-83, wherein the nucleic acid encoding the selection agent or the detection agent is integrated in an exon or an intron of KRT8.

Embodiment 83z. The method of embodiment 83y, wherein the nucleic acid encoding the selection agent or the detection agent is integrated in an exon of KRT8, optionally wherein the exon is exon 1, 2, 3, 4, 5, 6, 7, or 8.

Embodiment 83aa. The method of embodiment 83y, wherein the nucleic acid encoding the selection agent or the detection agent is integrated in an intron of KRT8, optionally wherein the intron is intron 1, 2, 3, 4, 5, 6, or 7.

Embodiment 83bb. The method of embodiment 82, wherein the nucleic acid encoding the selection agent or the detection agent is integrated in a 5′ UTR or a 3′ UTR of KRT8.

Embodiment 83cc. The method of any one of embodiments 50-51g, 53-56, 59-68, and 80-83, wherein the nucleic acid encoding the selection agent or the detection agent is integrated in an exon or an intron of CD9.

Embodiment 83dd. The method of embodiment 83cc, wherein the nucleic acid encoding the selection agent or the detection agent is integrated in an exon of CD9, optionally wherein the exon is exon 1, 2, 3, 4, 5, 6, 7, or 8.

Embodiment 83ee. The method of embodiment 83cc, wherein the nucleic acid encoding the selection agent or the detection agent is integrated in an intron of CD9, optionally wherein the intron is intron 1, 2, 3, 4, 5, 6, or 7.

Embodiment 83ff. The method of embodiment 82, wherein the nucleic acid encoding the selection agent or the detection agent is integrated in a 5′ UTR or a 3′ UTR of CD9.

Embodiment 83gg. The method of any one of embodiments 50-51g, 53-56, 59-68, and 80-83, wherein the nucleic acid encoding the selection agent or the detection agent is integrated in an exon or an intron of PLK2.

Embodiment 83hh. The method of embodiment 83gg, wherein the nucleic acid encoding the selection agent or the detection agent is integrated in an exon of PLK2, optionally wherein the exon is exon 1, 2, 3, 4, 5, 6, 7, 8, 9, 10, 11, 12, 13, or 14.

Embodiment 83ii. The method of embodiment 83gg, wherein the nucleic acid encoding the selection agent or the detection agent is integrated in an intron of PLK2, optionally wherein the intron is intron 1, 2, 3, 4, 5, 6, 7, 8, 9, 10, 11, 12, or 13.

Embodiment 83jj. The method of embodiment 82, wherein the nucleic acid encoding the selection agent or the detection agent is integrated in a 5′ UTR or a 3′ UTR of PLK2.

Embodiment 83kk. The method of any one of embodiments 50-51g, 53-56, 59-68, and 80-83, wherein the nucleic acid encoding the selection agent or the detection agent is integrated in an exon or an intron of KRT17.

Embodiment 83ll. The method of embodiment 83kk, wherein the nucleic acid encoding the selection agent or the detection agent is integrated in an exon of KRT17, optionally wherein the exon is exon 1, 2, 3, 4, 5, 6, 7, or 8.

Embodiment 83 mm. The method of embodiment 83kk, wherein the nucleic acid encoding the selection agent or the detection agent is integrated in an intron of KRT17, optionally wherein the intron is intron 1, 2, 3, 4, 5, 6, or 7.

Embodiment 83nn. The method of embodiment 82, wherein the nucleic acid encoding the selection agent or the detection agent is integrated in a 5′ UTR or a 3′ UTR of KRT17.

Embodiment 84. The method of any one of embodiments 0-51g, 53-56, 59-68, and 80-83nn, wherein:
(i) the off-target cell marker gene is ANXA1 and the selection agent is iCaspase9;
(ii) the off-target cell marker gene is KRT19 and the selection agent is iCaspase9;
(iii) the off-target cell marker gene is CTSC and the selection agent is iCaspase9;
(iv) the off-target cell marker gene is DSC2 and the selection agent is iCaspase9;
(v) the off-target cell marker gene is ARHGAP29 and the selection agent is iCaspase9;
(vi) the off-target cell marker gene is KRT18 and the selection agent is iCaspase9;
(vii) the off-target cell marker gene is KRT8 and the selection agent is iCaspase9;
(viii) the off-target cell marker gene is CD9 and the selection agent is iCaspase9;
(ix) the off-target cell marker gene is PLK2 and the selection agent is iCaspase9;
(x) the off-target cell marker gene is KRT17 and the selection agent is iCaspase9;
(xi) the off-target cell marker gene is ANXA1 and the selection agent is cytosine deaminase;
(xii) the off-target cell marker gene is KRT19 and the selection agent is cytosine deaminase;
(xiii) the off-target cell marker gene is CTSC and the selection agent is cytosine deaminase;
(xiv) the off-target cell marker gene is DSC2 and the selection agent is cytosine deaminase;
(xv) the off-target cell marker gene is ARHGAP29 and the selection agent is cytosine deaminase;
(xvi) the off-target cell marker gene is KRT18 and the selection agent is cytosine deaminase;
(xvii) the off-target cell marker gene is KRT8 and the selection agent is cytosine deaminase;
(xviii) the off-target cell marker gene is CD9 and the selection agent is cytosine deaminase;
(xix) the off-target cell marker gene is PLK2 and the selection agent is cytosine deaminase; or
(xx) the off-target cell marker gene is KRT17 and the selection agent is cytosine deaminase.

Embodiment 85. The method of any one of embodiments 0-51g, 53-56, 59-68, and 80-84, wherein the off-target cell marker gene is not a beta cell marker gene, a T cell marker gene, a neuronal cell marker gene, a glial cell marker gene, a cardiac cell marker gene, a retinal pigment epithelium (RPE) cell marker gene, a hematopoietic progenitor cell marker gene, a natural killer cell marker gene, an endothelial cell marker gene, or a lung cell marker gene.

Embodiment 86. A kit comprising a cell comprising the cell of any one of embodiments 1-11 and 19-19r and instructions for use.

Embodiment 87. A kit comprising a cell comprising the cell of any one of embodiments 12-19r and instructions for use.

Embodiment 88. The kit of embodiment 86 or 87, wherein the kit further comprises an inducer.

Embodiment 89. The method of any one of embodiments 22-85, and the kit of any one of embodiments 86-88, wherein the inducer is selected from the group consisting of rimiducid (AP1903), AP20187, rapamycin, 5-fluorocytosine, ganciclovir, CB 1954, 6-methylpurine deoxyriboside, fludarabine, indole-3-acetic acid (IAA), tetracycline, doxycycline, tamoxifen, cumate, FKCsA, abscisic acid (ABA), riboswitch, ecdysone, tryptophan, arabinose, isopropyl β-d-1-thiogalactopyranoside (IPTG), anti-MHC-I antibodies, anti-MHC-II antibodies, anti-CCR4 antibodies, anti-CD16 antibodies, anti-CD19 antibodies, anti-CD20 antibodies, anti-CD30 antibodies, anti-EGFR antibodies, anti-GD2 antibodies, anti-HER1 antibodies, anti-HER2 antibodies, anti-MUC1 antibodies, anti-PSMA antibodies, and anti-RQR8 antibodies.

Embodiment 90. The cell of any one of embodiments 1-19r, the method of any one of embodiments 22-85 and 89, and the kit of any one of embodiments 86-89, wherein the selection agent is a kill switch.

Embodiment 91. The cell of any one of embodiments 1-19r and 90, the method of any one of embodiments 22-85, 89, and 90, and the kit of any one of embodiments 86-90, wherein the kill switch is selected from the group consisting of an inducible caspase 9 (iCasp9), cytosine deaminase (CDA), herpes simplex virus thymidine kinase (HSV-Tk), rapamycin-activated caspase 9 (rapaCasp9), nitroreductase (NTR), purine nucleoside phosphorylase (PNP), horseradish peroxidase, inducible MHC-I, inducible MHC-II, CCR4, CD16, CD19, CD20, CD30, EGFR, GD2, HER1, HER2, MUC1, PSMA, and RQR8.

Embodiment 92. The cell of embodiment 91, the method of embodiment 91, and the kit of embodiment 91, wherein the kill switch is iCasp9 or CDA.

Embodiment 93. The cell of any one of embodiments 1-19r and 90-92, the method of any one of embodiments 22-85 and 89-92, and the kit of any one of embodiments 86-92, wherein the inducible MHC-I is selected from the group consisting of HLA-A, HLA-B, and HLA-C.

Embodiment 94. The cell of any one of embodiments 1-19r and 90-93, the method of any one of embodiments 22-85 and 89-93, and the kit of any one of embodiments 86-93, wherein the inducible MHC-II is selected from the group consisting of HLA-DP, HLA-DQ, and HLA-DR.

Embodiment 95. The cell of any one of embodiments 1-19r and 90-94, the method of any one of embodiments 22-85 and 89-94, and the kit of any one of embodiments 86-94, wherein the detection agent is a cell surface protein.

Embodiment 96. The cell of any one of embodiments 1-19r and 90-95, the method of any one of embodiments 22-85 and 89-95, and the kit of any one of embodiments 86-95, wherein the cell surface protein is selected from the group consisting of EGFR fused to a His-tag, RQRB fused to a His-tag, and CD47 fused to a His-tag.

Embodiment 97. The cell of any one of embodiments 1-19r and 90-96, the method of any one of embodiments 22-85 and 89-96, and the kit of any one of embodiments 86-96, wherein the detection agent is a fluorescent protein.

Embodiment 98. The cell of any one of embodiments 1-19r and 90-97, the method of any one of embodiments 22-85 and 89-97, and the kit of any one of embodiments 86-97, wherein the fluorescent protein is selected from the group consisting of: green fluorescent protein (GFP), enhanced green fluorescent protein (EGFP), yellow fluorescent protein (YFP), enhanced yellow fluorescent protein (EYFP), blue fluorescent protein (BFP), enhanced blue fluorescent protein (EBFP), cyan fluorescent protein (CFP), enhanced cyan fluorescent protein (ECFP), superfolder GFP, superfolder YFP, orange fluorescent protein, red fluorescent protein, small ultrared fluorescent protein, FMN-binding fluorescent protein, dsRed, qFP611, Dronpa, TagRFP, KFP, EosFP, IrisFP, Dendra, Kaede, KikGrl, emerald fluorescent protein, Azami Green, mWasabi, TagGFP, TurboGFP, AcGFP, ZsGreen, and T-Sapphire.

Embodiment 99. The cell of any one of embodiments 19r and 90-98, the method of any one of embodiments 22-85 and 89-98, and the kit of any one of embodiments 86-98, wherein the cell is a mammalian cell.

Embodiment 100. The cell of embodiment 99, the method of embodiment 99, and the kit of embodiment 99, wherein the mammalian cell is a human cell.

Embodiment 101. The cell of any one of embodiments 1-19r and 90-100, the method of any one of embodiments 22-85 and 89-100, and the kit of any one of embodiments 86-100, wherein the cell is a stem cell-derived cell.

Embodiment 102. The cell of embodiment 101, the method of embodiment 101, and the kit of embodiment 101, wherein the stem cell-derived cell is derived from a cell selected from the group consisting of embryonic stem cell, induced pluripotent stem cell, multipotent stem cell, adult stem cell, hematopoietic stem cell, mesenchymal stem cell, endothelial stem cell, epithelial stem cell, adipose stem or progenitor cells, germline stem cells, lung stem or progenitor cells, mammary stem cells, olfactory adult stem cells, hair follicle stem cells, multipotent stem cells, amniotic stem cells, cord blood stem cells, neural stem, and progenitor cells.

Embodiment 103. The cell, method, or kit of embodiment 101 or 102, wherein the stem-cell derived cell is selected from the group consisting of a stem cell-derived beta cell, an alpha cell, and a delta cell.

Embodiment 103a. The cell of embodiment 103, the method of embodiment 103, and the kit of embodiment 103, wherein the stem-cell derived cell is a stem cell-derived beta cell (SC-beta cell).

Embodiment 103b. The cell of embodiment 103a, the method of embodiment 103a, and the kit of embodiment 103a wherein the stem-cell derived cell is a stem cell-derived beta islet cell.

Embodiment 103c. The cell of embodiment 103a, the method of embodiment 103a, and the kit of embodiment 103a wherein the stem-cell derived cell is a stem cell-derived T cell.

Embodiment 103d. The cell of embodiment 103c, the method of embodiment 103c, and the kit of embodiment 103c, wherein the stem cell-derived T cell is selected from the group consisting of an ab T cell, dg T cell, helper/regulatory T cell, cytotoxic T cell, progenitor T cell (e.g., a progenitor T cell that is CD34+CD7+CD1a− or CD34+CD7+CD5+CD1a−), naive T cell, central memory T cell, effector T cell, terminal effector T cell, immature T cell, mature T cell, natural killer T cell, naive T cell, naive central memory T cell (TCM cell), effector memory T cell (TEM cell), and effector memory RA T cell (TEMRA cell).

Embodiment 103e. The cell of embodiment 103a, the method of embodiment 103a, and the kit of embodiment 103a wherein the stem-cell derived cell is a stem cell-derived neural cell.

Embodiment 103f. The cell of embodiment 103e, the method of embodiment 103e, and the kit of embodiment 103e, wherein the stem cell-derived neural cell is selected from the group consisting of a glial cell, cerebral endothelial cell, neuron, ependymal cell, astrocyte, microglial cell, oligodendrocyte, and a Schwann cell.

Embodiment 103g. The cell of embodiment 103a, the method of embodiment 103a, and the kit of embodiment 103a wherein the stem-cell derived cell is a stem cell-derived cardiac cell.

Embodiment 103h. The cell of embodiment 103c, the method of embodiment 103c, and the kit of embodiment 103c, wherein the stem cell-derived cardiac cell is selected from the group consisting of cardiomyocytes, nodal cardiomyocytes, conducting cardiomyocytes, working cardiomyocytes, cardiomyocyte precursors, cardiomyocyte progenitor cell, cardiac stem cell, and cardiac muscle cells.

Embodiment 104. The cell of embodiments 1-19r, 103a, and 103b, the method of embodiment 103a or 103b, and the kit of embodiment 103a or 103b, wherein the cells comprise one or more modifications that:
(a) inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules, and/or
(b) increase expression of one or more tolerogenic factors, wherein the increased expression is relative to a control or wild-type cell that does not comprise the modifications.

Embodiment 105. The cell of embodiments 1-19r and 104, the method of embodiment 104, and the kit of embodiment 104, wherein the one or more modifications in (i) reduce expression of:
a. one or more MHC class I molecules
b. one or more MHC class II molecules; or
c. one or more MHC class I molecules and one or more MHC class II molecules.

Embodiment 106. The cell of embodiments 1-19r, 104 and 105, the method of embodiment 104 or 105, and the kit of embodiment 104 or 105, wherein the one or more modifications in (i) reduce expression of one or more molecules selected from the group consisting of B2M, TAP I, NLRC5, CITTA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, and any combination thereof.

Embodiment 107. The cell of embodiments 1-19r and 106, the method of embodiment 106, and the kit of embodiment 106, wherein the modified SC-beta cell does not express one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, and combinations thereof.

Embodiment 108. The cell of any one of embodiments 1-19r and 105-107, the method of any one of any one of embodiments 105-107, and the kit of any one of embodiments 105-107, wherein the one or more modifications that increase expression comprise increased cell surface expression, and/or the one or more modifications that reduce expression comprise reduced cell surface expression.

Embodiment 109. The cell of any one of embodiments 1-19r and 104-108, the method of any one of embodiments 104-108, and the kit of any one of embodiments 104-108, wherein the one or more modifications in (i) reduce expression of one or more MHC class I molecules.

Embodiment 110. The cell of any one of embodiments 1-19r and 104-109, the method of any one of embodiments 104-109, and the kit of any one of embodiments 104-108, wherein the one or more molecules that regulate cell surface protein expression of the one or more MHC class I molecules are B2M.

Embodiment 111. The cell of any one of embodiments 1-19r and 104-110, the method of any one of embodiments 104-110, and the kit of any one of embodiments 104-110, wherein the modifications comprise a modification that regulates cell surface protein expression of the one or more MHC class I molecules and the modification inactivates or disrupts one or more alleles of B2M.

Embodiment 112. The cell of any one of embodiments 1-19r and 104-111, the method of any one of embodiments 104-111, and the kit of any one of embodiments 104-111, wherein the modification that inactivates or disrupts one or more alleles of B2M reduces mRNA expression of the B2M gene.

Embodiment 113. The cell of any one of embodiments 1-19r and 104-112, the method of any one of embodiments 104-112, and the kit of any one of embodiments 104-112, wherein the modification that inactivates or disrupts one or more alleles of B2M reduces protein expression of B2M.

Embodiment 114. The cell of any one of embodiments 1-19r and 104-113, the method of any one of embodiments 104-113, and the kit of any one of embodiments 104-113, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the cell.

Embodiment 115. The cell of any one of embodiments 1-19r and 104-114, the method of any one of embodiments 104-114, and the kit of any one of embodiments 104-114, wherein the inactivation or disruption comprises an indel in the B2M gene.

Embodiment 116. The cell of any one of embodiments 1-19r and 104-115, the method of any one of embodiments 104-115, and the kit of any one of embodiments 104-115, wherein the inactivation or disruption comprises a frameshift mutation or a deletion of a contiguous stretch of genomic DNA of the B2M gene.

Embodiment 117. The cell of any one of embodiments 1-19r and 104-116, the method of any one of embodiments 104-116, and the kit of any one of embodiments 104-116, wherein the one or more modifications in (i) reduce expression of HLA-A, HLA-B, and/or HLA-C.

Embodiment 118. The cell of any one of embodiments 1-19r and 104-117, the method of any one of embodiments 104-117, and the kit of any one of embodiments 104-117, wherein the one or more modifications in (i) reduce expression of one or more MHC class II molecules.

Embodiment 119. The cell of any one of embodiments 1-19r and 104-118, the method of any one of embodiments 104-118, and the kit of any one of embodiments 104-118, wherein the modification is a modification that regulates expression of the one or more MHC class II molecules, and the modification inactivates or disrupts one or more alleles of CITTA.

Embodiment 120. The cell of any one of embodiments 1-19r and 119, the method of embodiment 119, and the kit of embodiment 119, wherein the modification that inactivates or disrupts one or more alleles of CITTA reduces protein expression of CIITA.

Embodiment 121. The cell of any one of embodiments 1-19r, 119, and 120, the method of embodiment 119 or 120, and the kit of embodiment 119 or 120, wherein the modification that inactivates or disrupts one or more alleles of CITTA comprises:
inactivation or disruption of one allele of the CITTA gene;
inactivation or disruption of both alleles of the CITTA gene; or
inactivation or disruption of all CIITA coding alleles in the cell.

Embodiment 122. The cell of any one of embodiments 1-19r and 119-121, the method of any one of embodiments 119-121, and the kit of any one of embodiments 119-121, wherein the inactivation or disruption comprises an indel in the CIITA gene.

Embodiment 123. The cell of any one of embodiments 1-19r and 119-122, the method of any one of embodiments 119-122, and the kit of any one of embodiments 119-122, wherein the inactivation or disruption is a frameshift mutation or a deletion of a contiguous stretch of genomic DNA of the CIITA gene.

Embodiment 124. The cell of any one of embodiments 1-19r and 104-123, the method of any one of embodiments 104-123, and the kit of any one of embodiments 104-123, wherein the one or more modifications in (i) reduce expression of HLA-DM, HLA-DO, HLA-DP, HLA-DQ, HLA-DR, RFX5, RFXANK, and/or RFXAP.

Embodiment 125. The cell of any one of embodiments 1-19r and 104-124, the method of any one of embodiments 104-124, and the kit of any one of embodiments 104-124, wherein expression of HLA-A, HLA-B, HLA-C, HLA-DP, HLA-DQ, and HLA-DR are reduced in the engineered hypoimmunogenic islets.

Embodiment 126. The cell of any one of embodiments 1-19r and 104-125, the method of any one of embodiments 104-125, and the kit of any one of embodiments 104-125, wherein the one or more tolerogenic factors is selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.

Embodiment 127. The cell of any one of embodiments 1-19r and 104-126, the method of any one of embodiments 104-126, and the kit of any one of embodiments 104-126, wherein at least one of the one or more tolerogenic factors is CD47.

Embodiment 128. The cell of any one of embodiments 1-19r and 104-127, the method of any one of embodiments 104-127, and the kit of any one of embodiments 104-127, wherein the one or more tolerogenic factors is CD47.

Embodiment 129. The cell of any one of embodiments 1-19r and 104-128, the method of any one of embodiments 104-128, and the kit of any one of embodiments 104-128, wherein the modification that increases expression of the one or more tolerogenic factors comprises an exogenous polynucleotide encoding the one or more tolerogenic factors.

Embodiment 130. The cell of any one of embodiments 1-19r and 129, the method of embodiment 129, and the kit of embodiment 129, wherein the exogenous polynucleotide encoding the one or more tolerogenic factors is integrated into the genome of the engineered hypoimmunogenic islets.

Embodiment 131. The cell of any one of embodiments 1-19r and 104-130, the method of any one of embodiments 104-130, and the kit of any one of embodiments 104-130, wherein the one or more tolerogenic factors comprises CD47 and the engineered hypoimmunogenic islets expresses CD47 at a first level that is greater than at or about 5-fold over a second level expressed by the control or wild-type islet cell.

Embodiment 132. The cell of any one of embodiments 1-19r and 131, the method of embodiment 131, and the kit of embodiment 131, wherein CD47 is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type islet cell.

Embodiment 133. The cell of any one of embodiments 1-19r and 104-132, the method of any one of embodiments 104-132, and the kit of any one of embodiments 104-132, wherein the one or more tolerogenic factors comprises CD47 and CD47 is expressed by the engineered hypoimmunogenic islets at greater than at or about 20,000 molecules per cell.

Embodiment 134. The cell of any one of embodiments 1-19r and 133, the method of embodiment 133, and the kit of embodiment 133, wherein CD47 is expressed by the engineered hypoimmunogenic islets at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

Embodiment 135. The cell of any one of embodiments 1-19r and 104-130, the method of any one of embodiments 104-130, and the kit of any one of embodiments 104-130, wherein the engineered hypoimmunogenic islets has the phenotype B2Mindel/indel; CIITAindel/indel; CD47tg.

Embodiment 136. The cell of any one of embodiments 1-19r and 104-135, the method of any one of embodiments 104-135, and the kit of any one of embodiments 104-135 wherein among the dose of cells engineered hypoimmunogenic islets at least 85% of the cells have the modifications.

Embodiment 137. The cell of any one of embodiments 1-19r and 136, the method of embodiment 136, and the kit of embodiment 136, wherein at least 90%, at least 92%, at least 95% or at least 98% of the cells have the modifications.

Embodiment 138. The cell of any one of embodiments 1-19r and 104-135, the method of any one of embodiments 104-135, and the kit of any one of embodiments 104-135, wherein among the dose of cells engineered hypoimmunogenic islets at least 85% of the cells have the phenotype has the phenotype B2Mindel/indel; CIITAindel/indel; CD47tg.

Embodiment 139. The cell of any one of embodiments 1-19r and 138, the method of embodiment 138, and the kit of embodiment 138, wherein at least 90%, at least 92%, at least 95% or at least 98% of the cells have the phenotype.

### EXAMPLES

The examples below are intended to be purely exemplary of the invention and should therefore not be considered to limit the invention in any way. The following examples and detailed description are offered by way of illustration and not by way of limitation.

### Example 1: Generation of Modified Cells Containing a Nucleic Acid Encoding an Agent (e.g., Kill Switch) Integrated at an Endogenous Gene Locus

This example describes the generation of modified cells containing a nucleic acid encoding an agent (e.g., a selection agent or a detection agent) integrated at specific gene loci to enable the expression of the agent, which will allow for the selective elimination or identification of a subset of the modified cells expressing the agent.

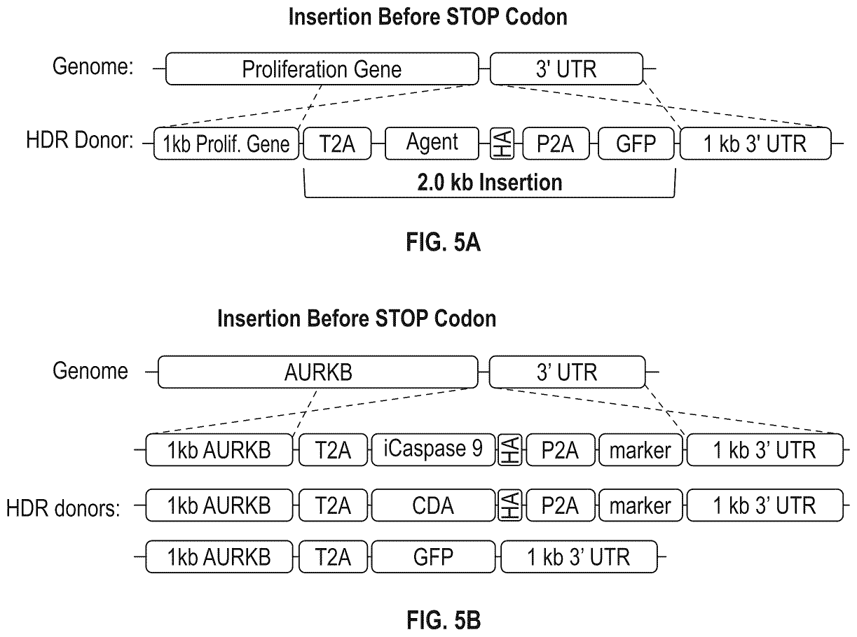

Genes of interest (e.g., cancer-associated genes or off-target genes) are identified using next-generation sequencing techniques and analyses of gene expression signatures. A nucleic acid encoding an agent (e.g., a selection agent or a detection agent) is engineered into the gene of interest via homology-directed repair (HDR) such that the gene of interest and the nucleic acid encoding an agent (e.g., a selection agent or a detection agent) are expressed from the same transcript (FIGS. 5A, 20, and 21). One or more detection agents can be integrated into the same gene locus. One or more detection agents can be integrated into different gene loci. One or more selection agents can be integrated into the same gene locus. One or more selection agents can be integrated into different gene loci. One detection agent and one selection agent can be integrated into the same gene locus. One detection agent and one selection agent can be integrated into different gene loci. In an exemplary example, the agent (e.g., a selection agent or a detection agent) is inserted into the 3′ UTR of the gene of interest. As proof-of-concept, the modified cells are tested in dose response experiments for the ability to eliminate or identify a subset of unwanted cells.

The modified cells containing a nucleic acid encoding an agent (e.g., a selection agent) may be subjected to an inducer, wherein the presence of the inducer causes elimination of a subset of modified cells. The modified cells containing a nucleic acid encoding an agent (e.g., a detection agent) may result in, for example, the surgical removal, purification of the cell population, or modification of a treatment regimen, to eliminate the subset of unwanted cells expressing the detection agent.

### Example 2: Engineering Safer Stem Cell-Derived Therapies

Stem cell derived therapies with immune evasion potential offer key advantages over autologous therapies including cell line selection, better manufacturing control, and wider patient access. To reduce oncological risk, most pluripotent stem cells (PSCs) must progress to the post-mitotic stage during differentiation; however, as few as 20 residual proliferating cells may cause tumors or teratomas that could evade the immune system. A schematic representation is shown in FIG. 1. To address this issue, “proliferation safety switches” were designed to be expressed only in proliferating cells by linking the expression of a safety switch to endogenous cancer-associated genes but are turned off in post-mitotic cells. In cases where the endogenous proliferation gene is expressed at a low level or above, the safety switch can be controlled by an inducer or activator, thereby reducing or preventing unintended impact of the safety switch. These switches could selectively eliminate proliferating cells in vitro or in vivo, enhancing safety and preserving differentiated therapeutic cells.

### In Silico Screen for Gene Candidates (e.g., Proliferation Gene Candidates)

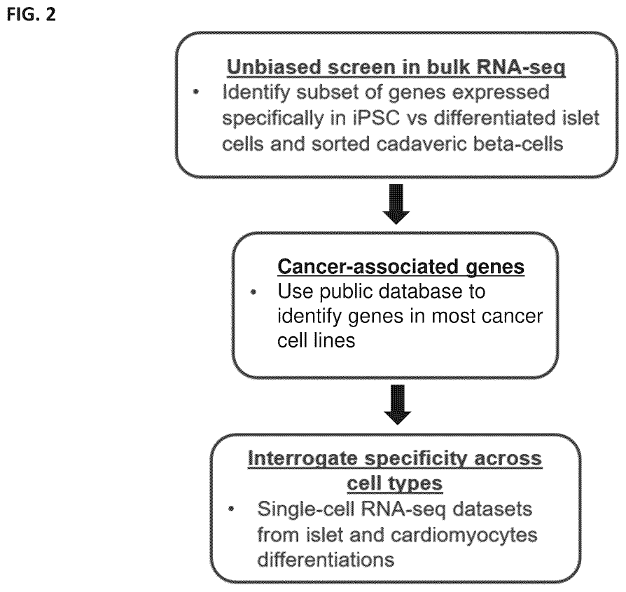

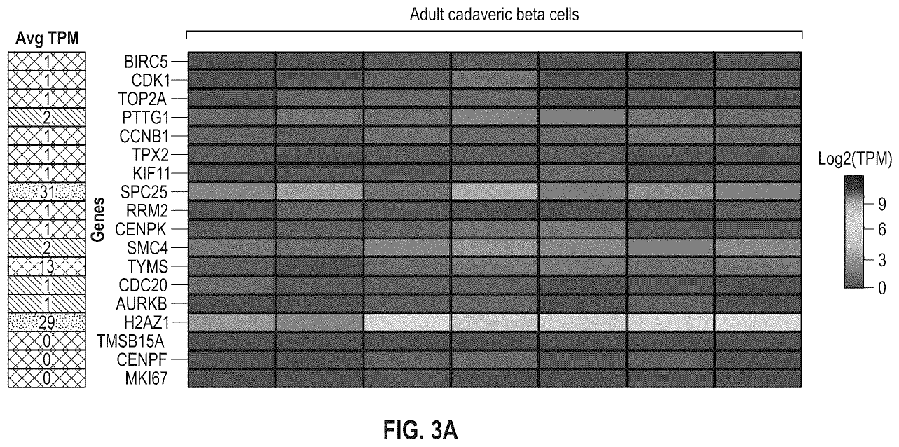

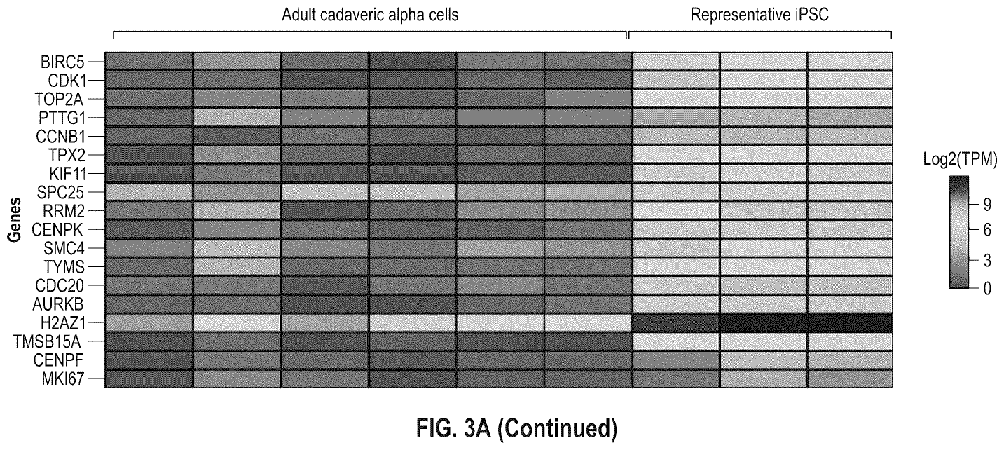

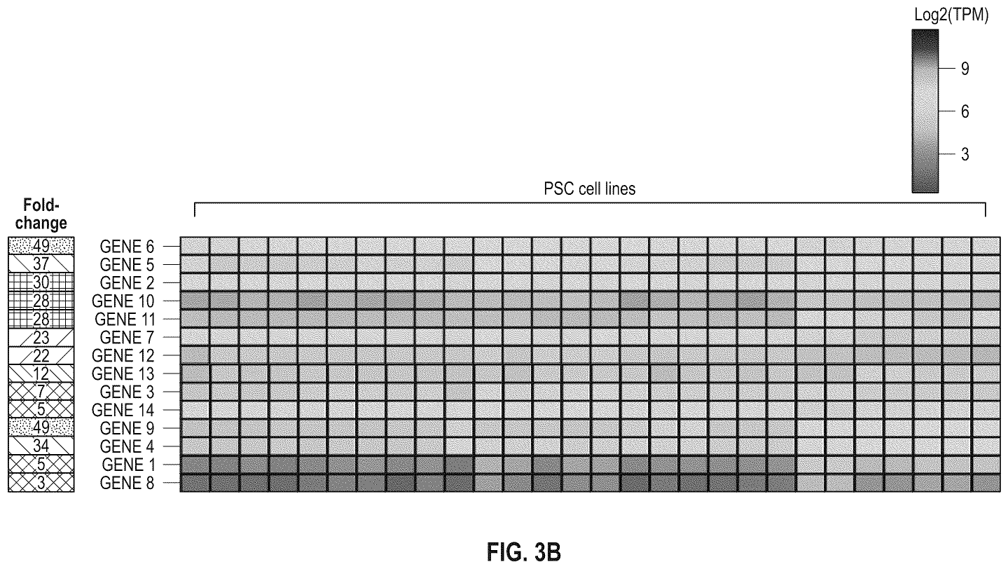

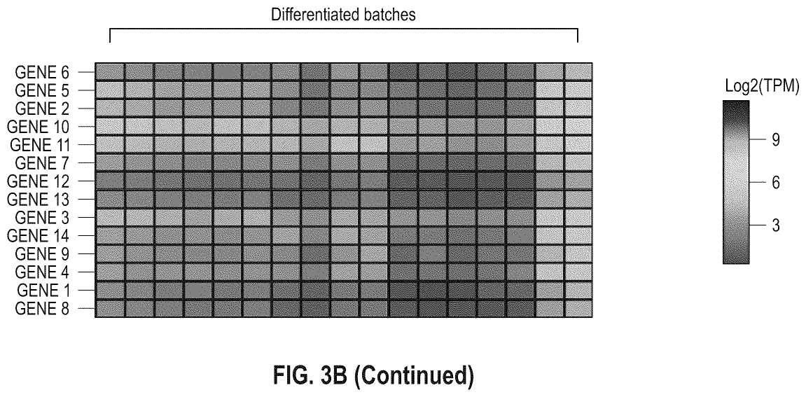

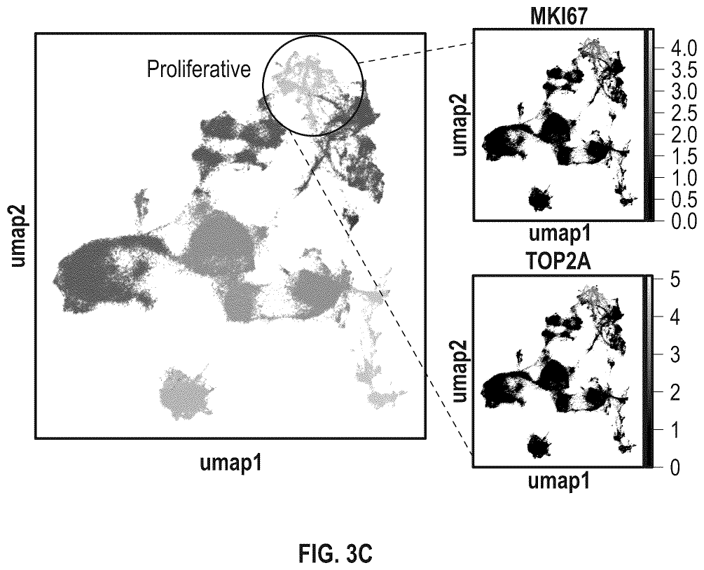

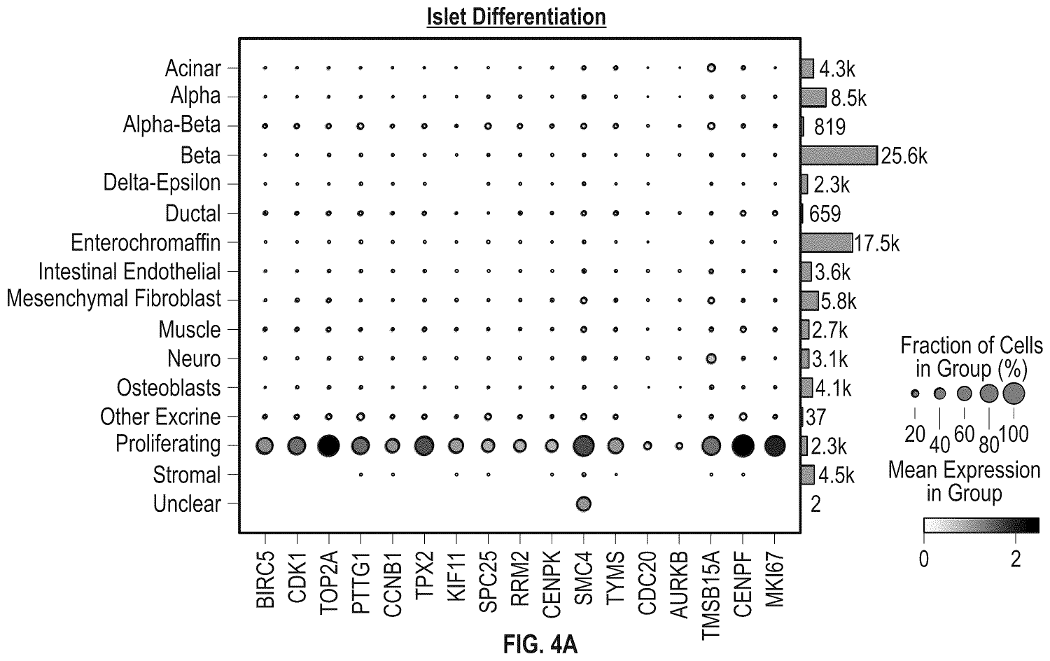

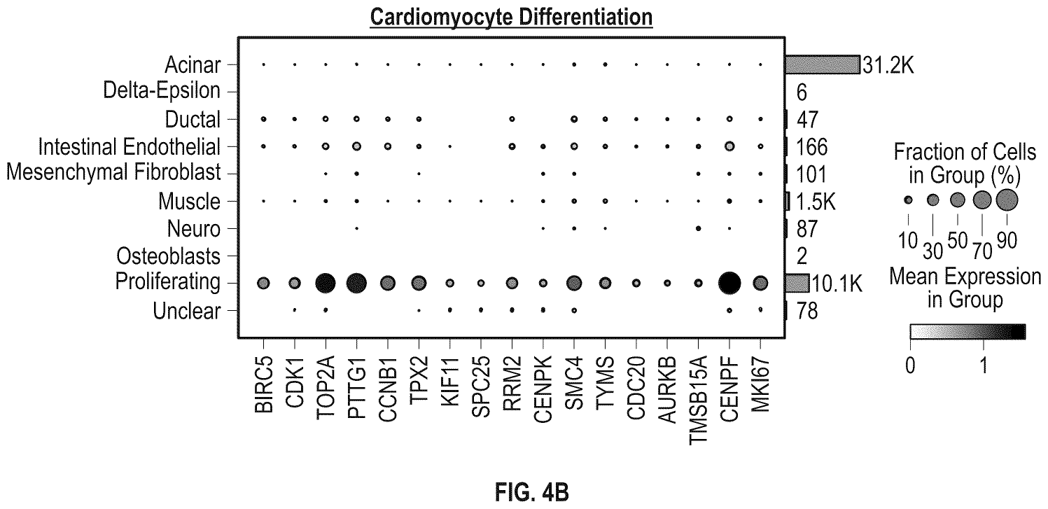

Exemplary proliferation genes were identified in silico. Genes associated with cancer lines were identified: selected genes included genes whose knockout resulted in cell death in at least half of cancer cell lines in a database. Additionally, single-cell RNA (scRNA) sequencing data of two different PSC-derived cell types (n=22) (FIG. 3C) as well as bulk RNA sequencing data of PSC (n=28) compared with PSC-derived and cadaveric-derived sorted cells (n=24) (FIG. 3B) were analyzed to select which of the genes behaved as “proliferation genes,” specifically expressed in pluripotent and proliferative cell types and not in terminally differentiated cells, agnostic of cell type (FIG. 2). Bulk RNA sequencing of adult cadaveric beta cells, adult cadaveric alpha cells, and representative induced PSCs (iPSCs) was used to identify genes with the largest fold change in expression among the datasets. As shown in FIG. 3A, sorted cadaveric islet cells do not express proliferation genes. The analysis was further refined using scRNA sequencing across annotated cell types to interrogate the specificity of nominated genes. scRNA sequencing data of beta islet (FIG. 4A) and cardiomyocyte (FIG. 4B) differentiations identified a gene signature of proliferative cells that was distinct from iPSCs. The selection criteria for proliferation gene candidates are outlined in Table 13 below. The screening approach yielded the following 14 candidate proliferation genes: CDC20, BIRC5, CDK1, CENPF, AURKB, TOP2A, PTTG1, TPX2, KIF11, SPC25, RRM2, CENPK, SMC4, and TYMS.

TABLE 13
Selection criteria for proliferation gene candidates.

| | | | | | | Gene | | | Rank |
|---|---|---|---|---|---|---|---|---|---|
| | | | | Absolute | | effect | | | score of |
| | Fold Change | TPM in | Absolute | TPM | Gene | score | TPM in | SC451 | DE genes |
| | iPSC/diff in | cadaveric | TPM | differentiated | dependency | in | cadaveric | CellXGene | in |
| Gene | bulk RNA-seq | cells | in PSC | batches | probability | knockdown | islets | score | scRNA-seq |
| CDC20 | 49 | 1 | 487 | 10 | 1 | −0.22 | 1 | N/A | N/A |
| BIRC5 | 49 | 0 | 261 | 5 | 0.99 | −0.55 | 1 | 0.23 | 0.33 |
| CDK1 | 37 | 1 | 173 | 5 | 1 | −1.01 | 1 | 0.14 | 0.29 |
| AURKB | 34 | 2 | 272 | 8 | 0.99 | −0.95 | 1 | N/A | N/A |
| TOP2A | 30 | 1 | 290 | 10 | 0.99 | −0.45 | 1 | 0.48 | 0.94 |
| PTTG1 | 28 | 1 | 309 | 11 | 0.55 | −0.08 | 1 | 0.25 | 0.52 |
| TPX2 | 23 | 1 | 236 | 10 | 0.94 | −0.33 | 1 | 0.22 | 0.42 |
| KIF11 | 22 | 1 | 84 | 4 | 1 | −0.44 | 1 | 0.12 | 0.21 |
| SPC25 | 15 | N/A | 71 | 5 | 0.99 | −1.25 | 31 | 0.13 | 0.19 |
| RRM2 | 12 | N/A | 84 | 7 | 1 | −0.6 | 1 | 0.16 | 0.22 |
| CENPK | 12 | 0 | 27 | 2 | 0.99 | −0.2 | 1 | 0.12 | 0.22 |
| SMC4 | 7 | 1 | 46 | 7 | 0.99 | −0.64 | 2 | 0.31 | 0.5 |
| TYMS | 5 | 2 | 17 | 3 | 0.55 | −0.51 | 13 | 0.12 | 0.52 |
| CENPF | 5 | 1 | 21 | 4 | 0.23 | 0.02 | 0 | 0.72 | 1.04 |

In Vitro Validation of Proliferation Targets (iPSCs and Differentiated Cells)

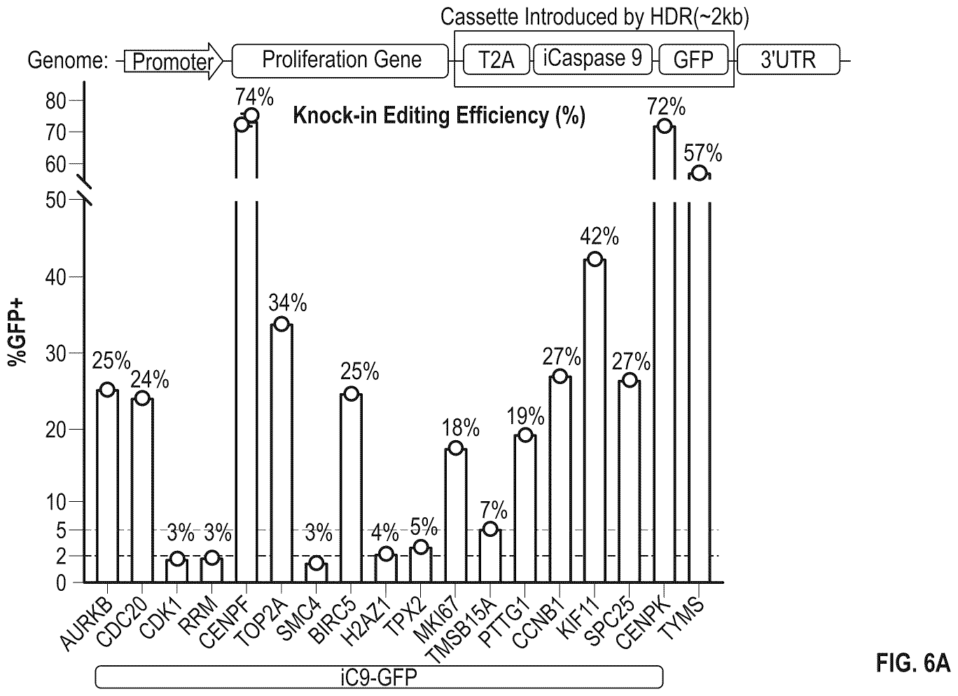

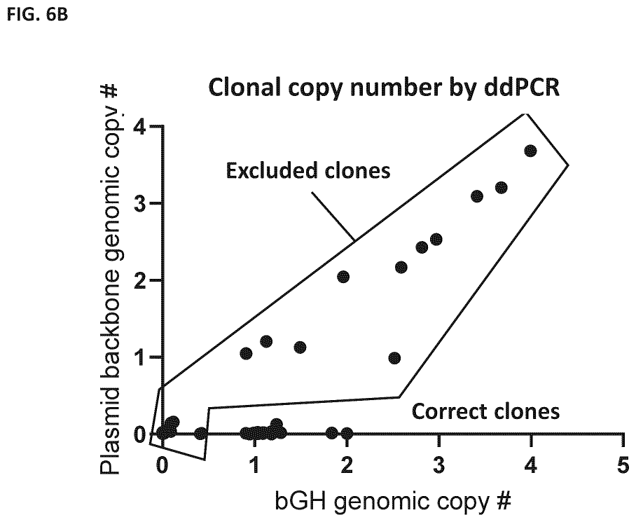

Endogenous loci in iPSCs were engineered via homology-directed repair (HDR) such that the proliferation gene and a kill switch that eliminates cells in the presence of a small molecule activator were expressed from the same transcript. To knock-in (KI) a kill switch at the endogenous proliferation gene locus, CRISPR/Cas guide RNA (gRNA) candidates were identified, homology arms were designed, and homology-directed repair (HDR) donors introduced the kill switch into the 3′ UTR of the endogenous proliferation gene locus (FIG. 5A). All available gRNA and HDR donor pairs were tested in a 30 bp window around the stop codon. In an exemplary example of CRISPR/Cas 9-mediated gene tagging of the AURKB locus, a promoter-less GFP reporter or kill switch (iCaspase 9 or cytosine deaminase) were introduced by HDR into the 3′ UTR (e.g., insertion before the stop codon) of the AURKB locus (FIG. 5B). A 2-kilobase (kb) inducible Caspase 9-GFP (iC9-GFP) kill switch was knocked in frame with the candidate genes in PSCs that then underwent single cell cloning. As measured by a GFP reporter, knock-in efficiency of iC9-GFP varied across loci. As shown in FIG. 6A, the CENPF, CENPK, and TYMS loci exhibited 74%, 72%, and 57% knock-in efficiency, respectively, while the other proliferation gene loci exhibited less than 50% knock-in efficiency. All proliferation targets exhibited knock-in efficiencies higher than 2% with Cas9, which permitted subsequent single cell cloning. As shown in FIG. 6B, ddPCR was used to screen for correctly edited iPSC clones. Edited iPSC clones with monoallelic or biallelic integration and without a plasmid backbone were selected for further characterization.

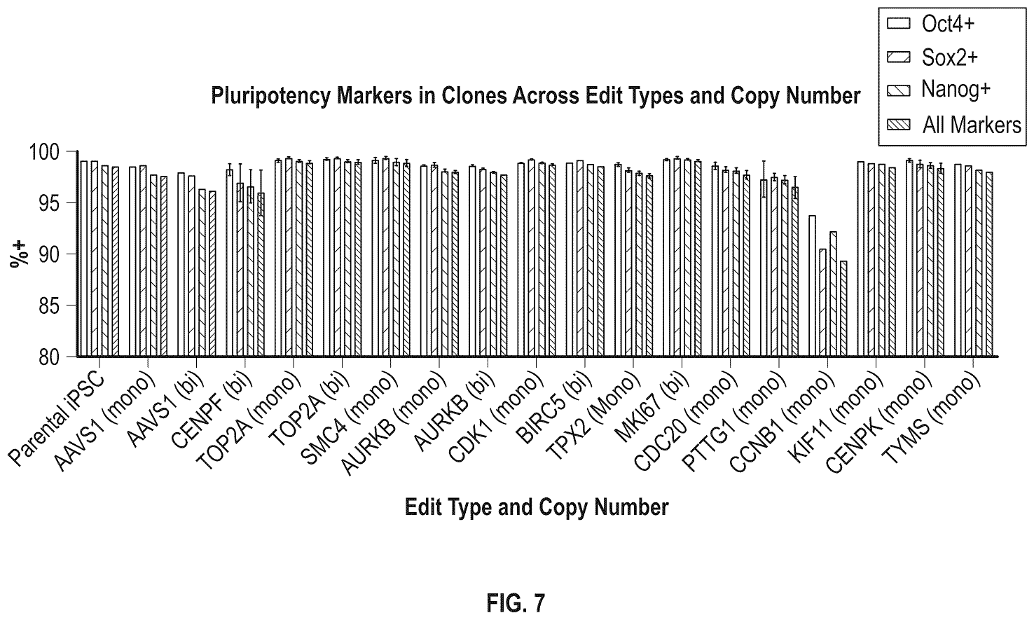

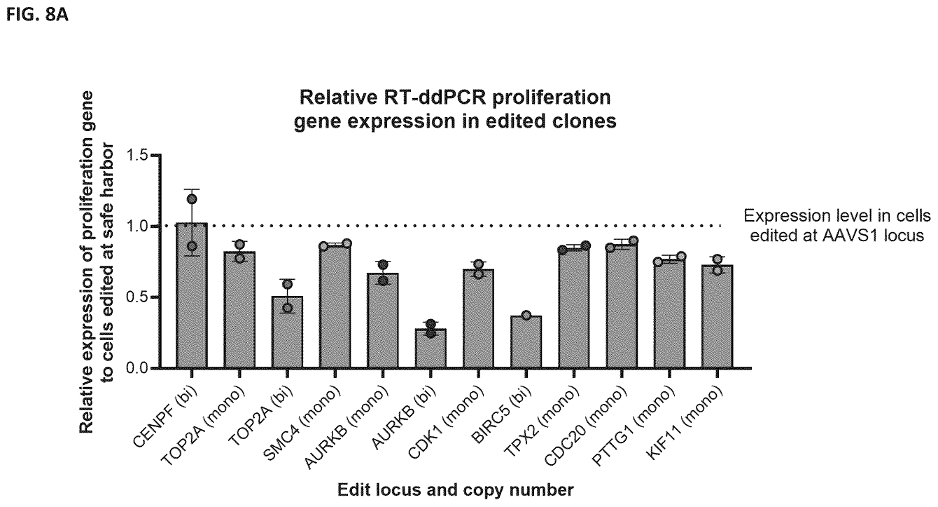

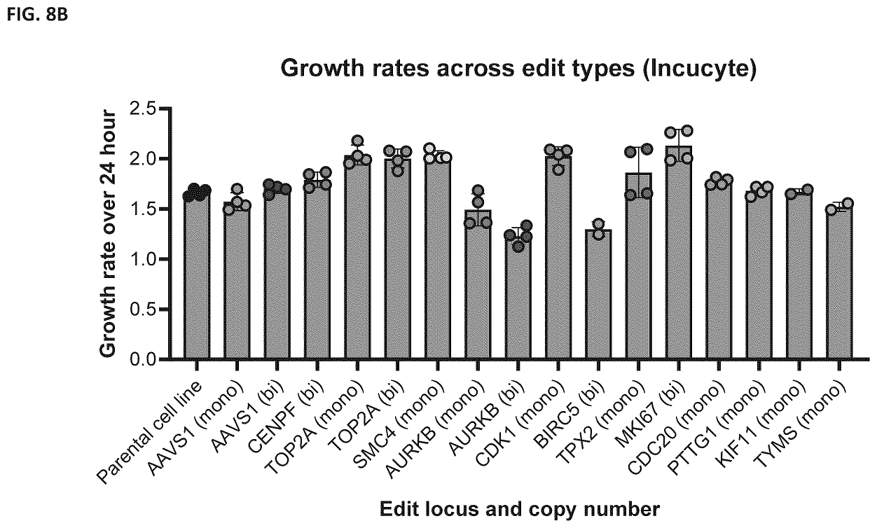

The proliferation kill switches were assessed throughout differentiation for kill switch and proliferation gene expression using flow cytometry, RT-ddPCR, and killing kinetics via viability assays. To determine whether iPSCs remained pluripotent after kill switches were knocked into the proliferation gene loci, flow cytometry was used to detect nuclear staining for pluripotency markers (Oct4, Sox2, and Nanog) of various iPSC lines containing either monoallelic or biallelic knock-in of iC9-GFP. All cell lines passed the 95% threshold for triple positivity of the pluripotency markers, except for the monoallelic CCNB1 clone (FIG. 7). Proliferation gene expression of knock-in cells was measured by RT-ddPCR. As shown in FIG. 8A, compared to expression of specific proliferation genes in control cells edited at the AAVS1 locus, most monoallelic knock-ins at the specified locus resulted in lower expression of the proliferation gene while most biallelic knock-ins, except for CENPF, resulted in an even greater decrease in expression of the proliferation gene. Incucyte live-cell imaging was used to track the proliferation of cells upon spontaneous differentiation. Compared to the parental iPSC line dko D11 (a pluripotent cell line-derived clone with a double knock-out for genes B2M and CIITA), most knock-in cells have unaffected growth rates measured over 24 hours (FIG. 8B). Taken together, although gene expression varied in the knock-in cells, genome engineering overall did not affect pluripotency and doubling times were on par with the parental line.

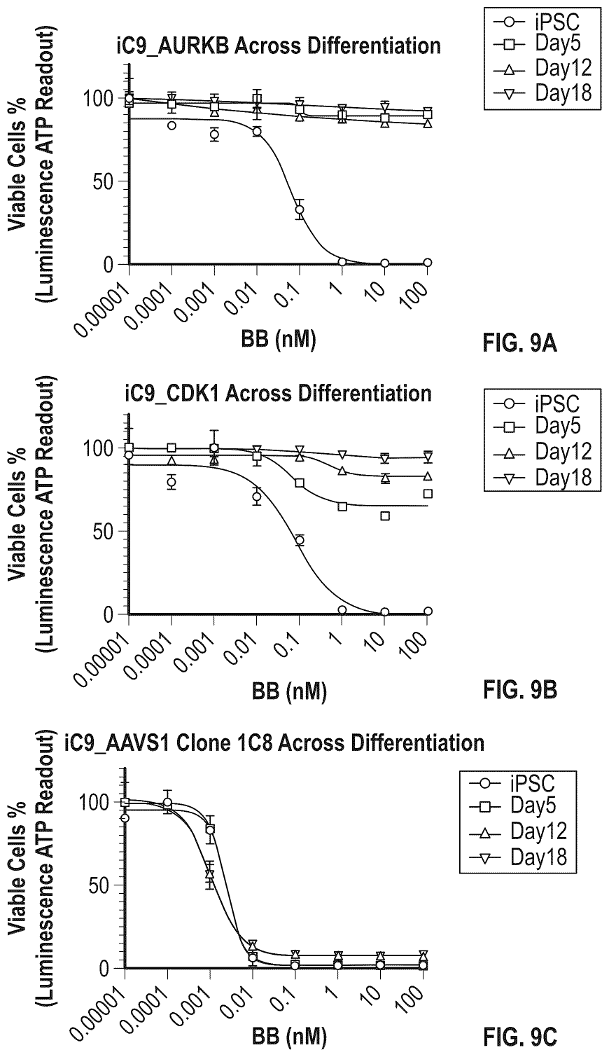

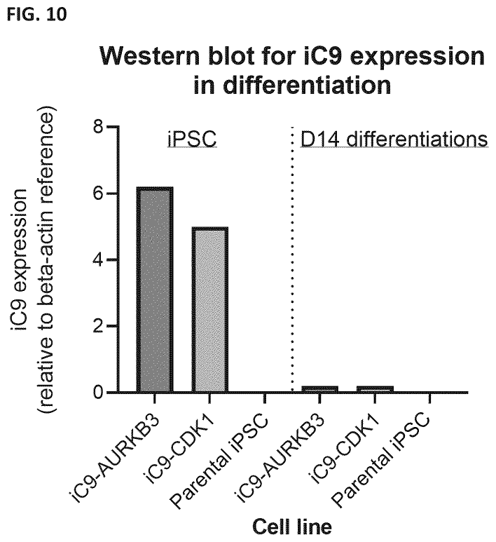

Cell killing analysis was performed to determine whether kill switch/safety switch activation by a small molecule inducer would lead to selective elimination of proliferative cells throughout an 18-day spontaneous iPSC differentiation model. iPSCs with iCaspase 9 inserted into cell cycle genes AURKB (iC9_AURKB), CDC20 (iC9_CDC20), or CDK1 (iC9_CDK1) with a puromycin marker, and then selected with 2 μM puromycin for 72 hours, were tested. As a control, an iPSC line was generated containing an iCaspase 9 inserted to the AAVS1 locus where the resulting iPSC had constitutive expression of iCaspase 9. Cell viability was measured at day 5, day 12, and day 18 of differentiation. As expected, there was no loss of iCaspase 9 function (i.e., iCaspase 9 was continually expressed) in control iC9_AAVS1 iPSCs. Activation/induction of iCaspase 9 with at least 0.01 nM AP20187 (“BB”) in iC9_AAVS1 iPSCs led to 0% cell viability across all stages of differentiation (FIG. 9C). In iC9_AURKB iPSCs, there was complete loss of iCaspase 9 function (due to the loss of AURKB expression) early in differentiation despite proliferating cells remaining in the iPSC culture (FIG. 9A). As shown in FIG. 9A, activation of the kill switch with 1 nM AP20187 in iC9_AURKB iPSCs led to no viable cells while nearly 100% of cells survived from day 5 of differentiation. Gradual loss of function of iCaspase 9 (corresponding to gradual loss of expression of CDK1) was observed for iC9_CDK1 iPSCs. Although there was complete cell death with 1 nM AP20187 at the iPSC stage, an increasing percentage of cells were viable at each later stage of differentiation (FIG. 9B). Consistent with the kill curves in FIGS. 9A and 9B, quantification of Western blotting of iCaspase 9 expression in iC9_AURKB and iC9_CDK1 cell lines showed iCaspase 9 expression decreased by 6-fold at day 14 of differentiation, compared to the iPSC stage (FIG. 10). This data demonstrated that although iCaspase 9 was expressed and capable of induced cell killing (i.e., was functional) when knocked into the AURKB, CDC20, and CDK1 loci, its expression decreased during differentiation corresponding to a decrease in function (e.g., cell killing).

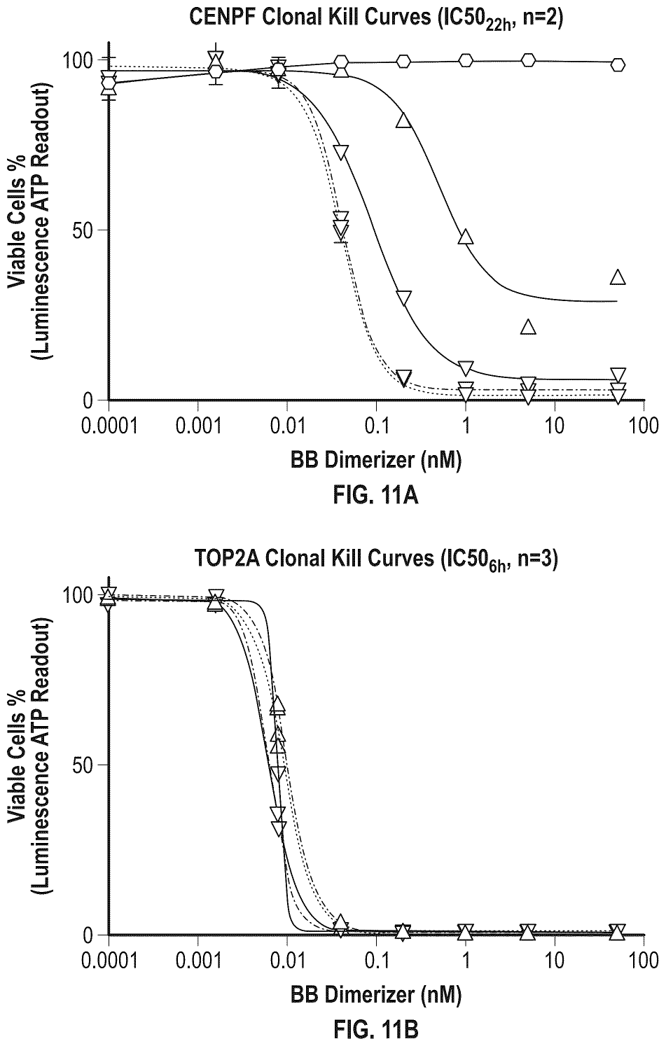

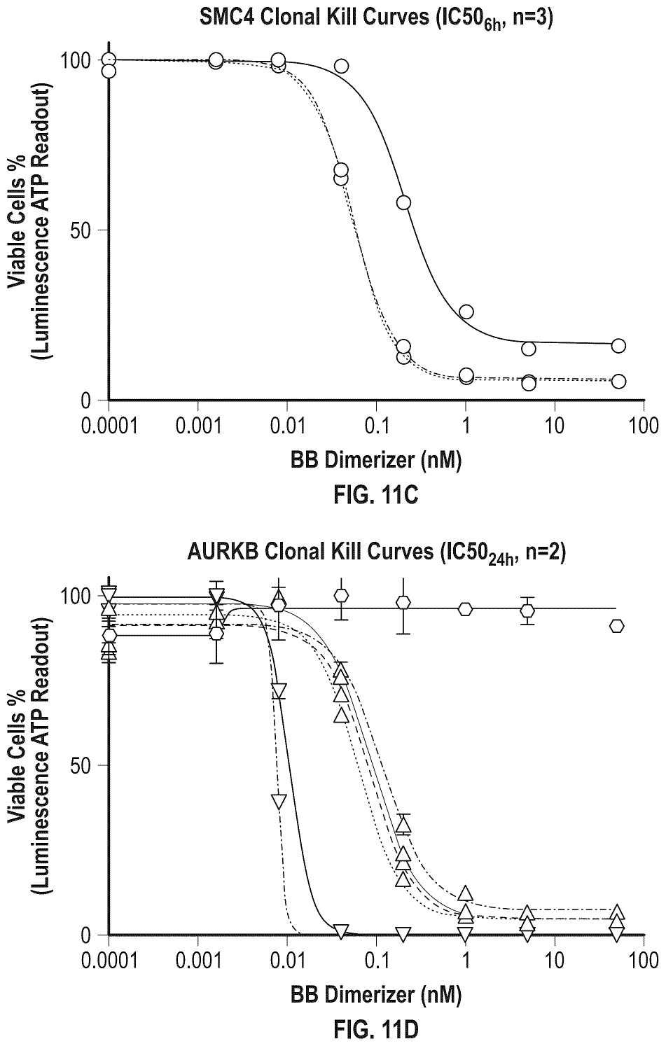

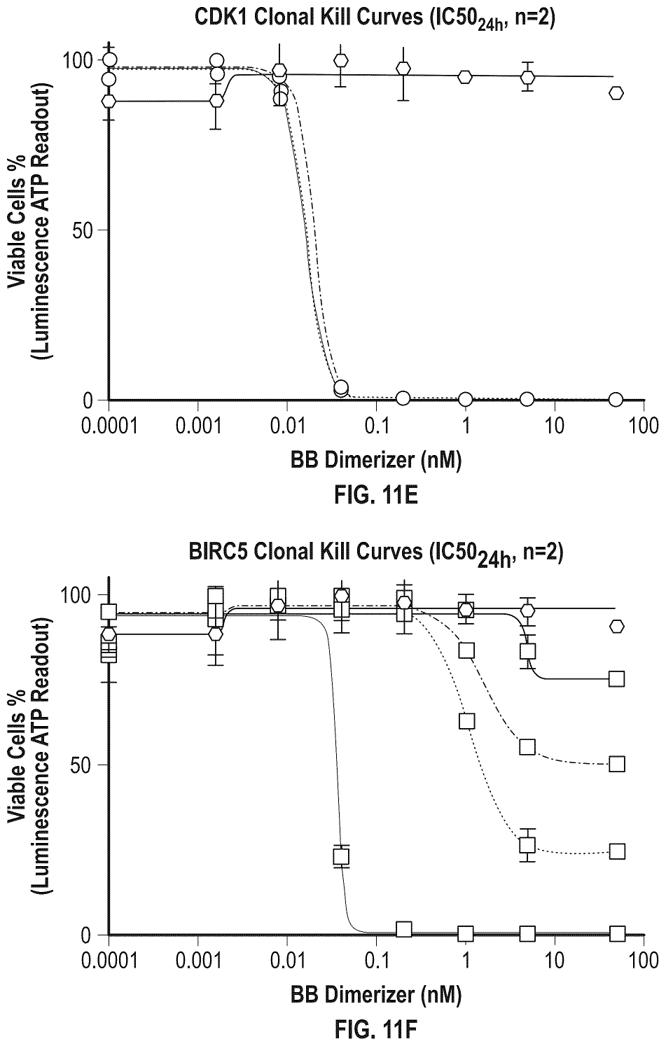

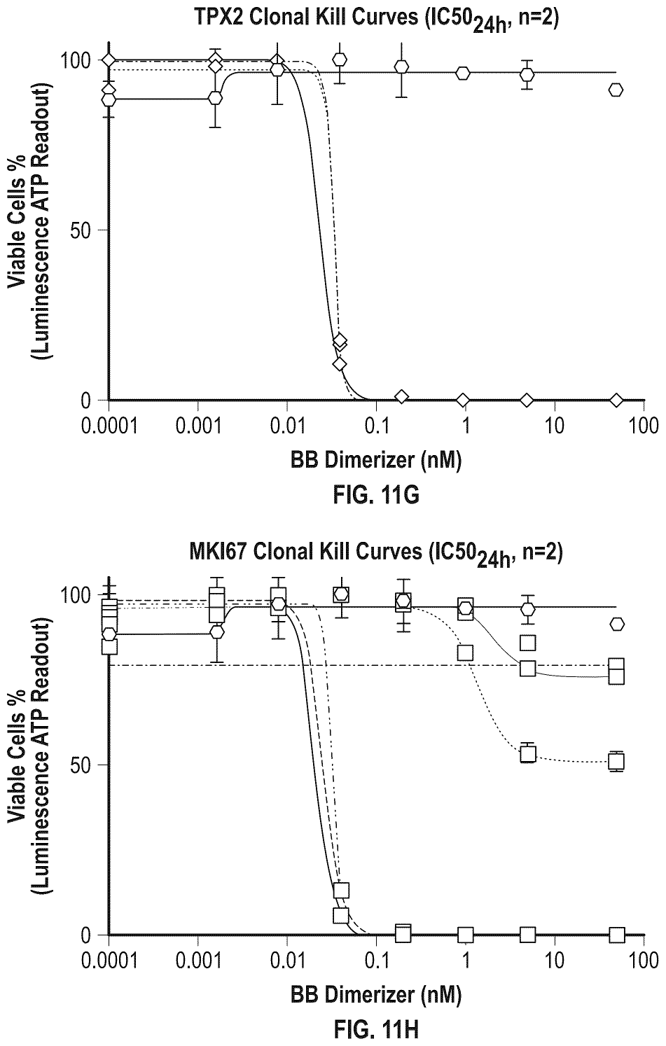

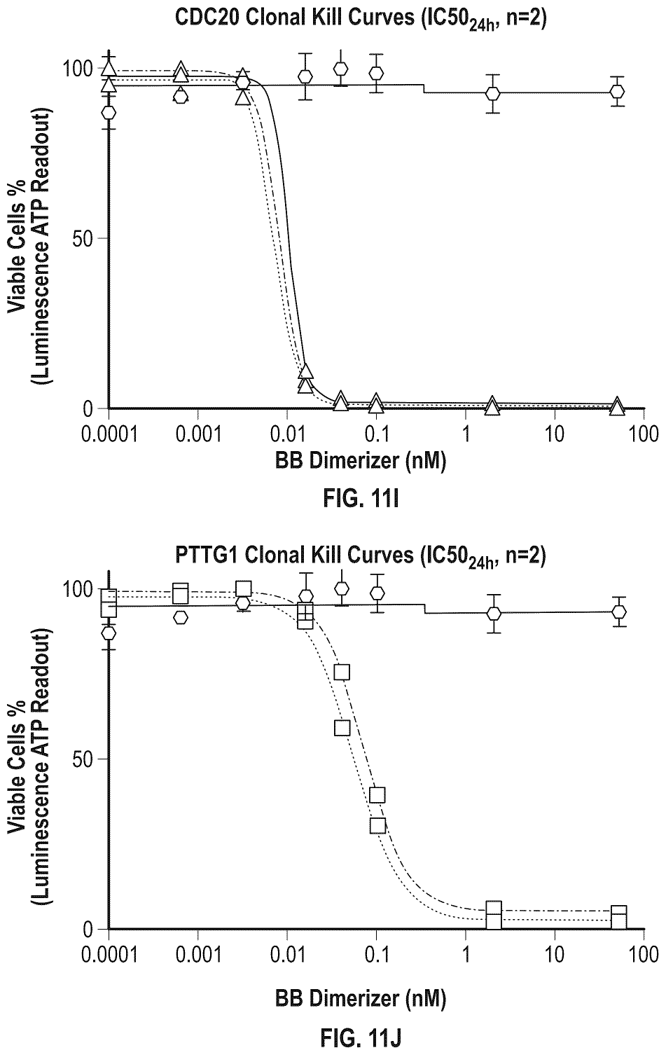

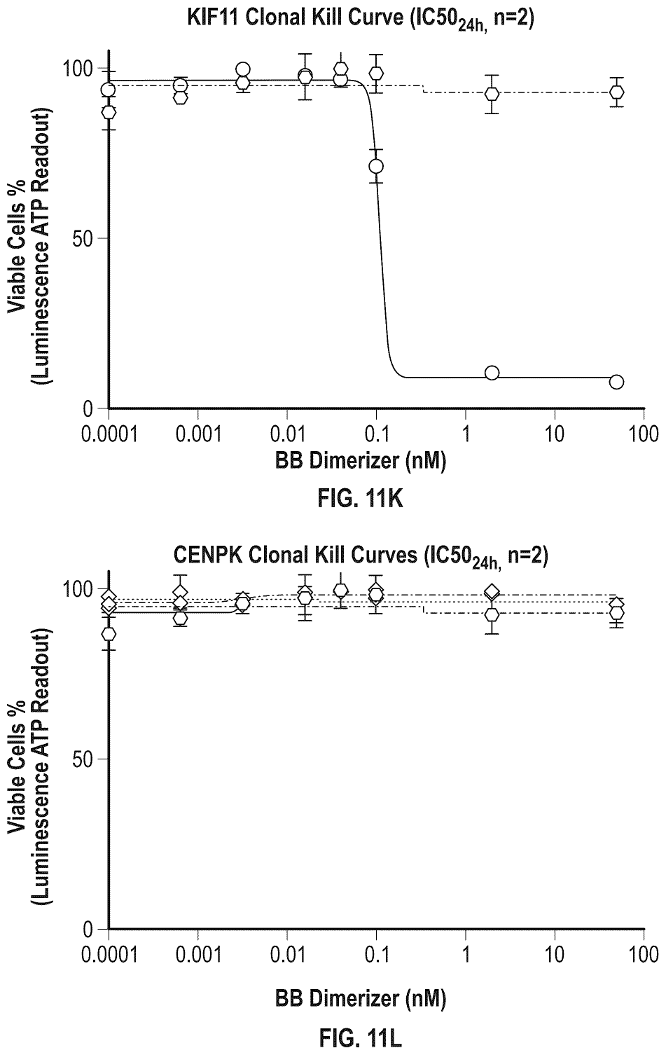

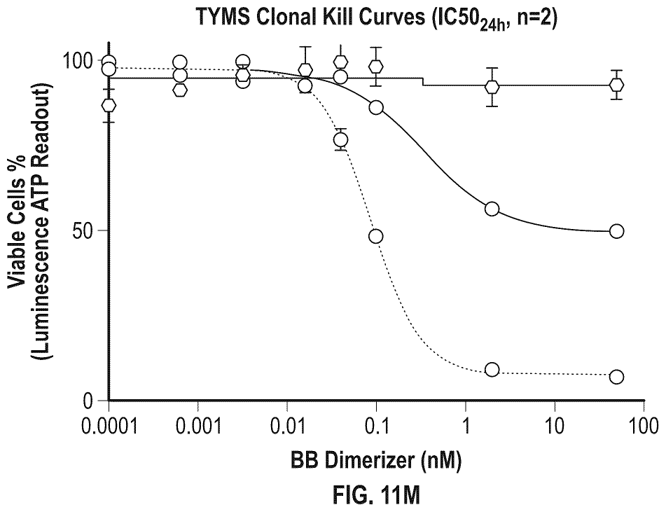

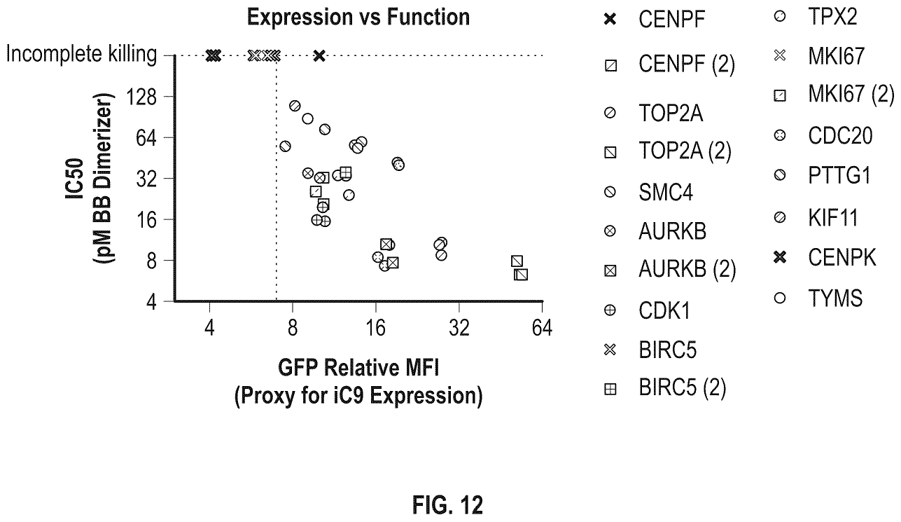

Cell lines with iC9-GFP knocked into proliferation gene loci were tested for kill switch function. After addition of AP20187 (“BB dimerizer”), cell viability was measured. iC9_TOP2A (FIG. 11B), iC9_SMC4 (FIG. 11C), iC9_AURKB (FIG. 11D), iC9_CDK1 (FIG. 11E), iC9_TPX2 (FIG. 11G), iC9_CDC20 (FIG. 11I), iC9_PTTG1 (FIG. 11J), and iC9_KIF11 (FIG. 11K) cell lines exhibited kill switch function, as evidenced by low or no cell viability. Monoallelic clones iC9_CENPF (FIG. 11A), iC9_BIRC5 (FIG. 11F), and iC9_MKI67 (FIG. 11H) demonstrated incomplete killing. As shown in FIG. 11L, iC9_CENPK did not achieve any cell killing, indicating loss of iCaspase 9 function when knocked into the CENPK locus. iC9_TYMS cell lines showed differing levels of cell viability due to clone variations (FIG. 11M). Overall, knock-in of iCaspase 9 into most proliferation gene loci conferred kill switch function in the clones. When iCaspase 9 expression, measured by the mean fluorescence intensity (MFI) of GFP, was plotted against the IC₅₀ for a specific proliferation gene KI clone, there was a minimum threshold of expression that determined iCaspase 9 function, with the exception of the CENPF KI clone (FIG. 12). The kill switch knock-in was functional in most proliferation gene candidates, and in some cases, depended on copy number. Some loci required biallelic integration to confer function, which correlated with the expression level of the kill switch. Therefore, iCaspase 9 expression level in iPSC clones correlated with the efficacy of killing.

In Vitro Validation of iCaspase 9 in a Spontaneous Differentiation Model

To determine whether iCaspase 9 function shut down (i.e., decrease iCaspase 9 expression) as differentiation progressed, the viability of different iC9 monoallelic and biallelic KI cell lines were tested for undifferentiated iPSCs and days 5 and 18 of iPSC differentiation. The growth rates of various iC9 KI cell lines were measured in order to establish a baseline understanding of the cells' proliferation timeline. As shown in FIG. 13, most iC9 KI clones, as well as the control parental iPSC line (dko), proliferate slower by day 5 of differentiation and were non-proliferative by day 18. As expected, the control parental iPSC line (dko) showed 100% cell viability across differentiation while both monoallelic and biallelic AAVS1 control clones remained stable with no viable cells (IC₅₀=0.9-2.6 pM) (FIG. 14A).

The kill curves for monoallelic iC9_CDK1 cell lines showed progressive loss of iCaspase 9 function (decreased iCaspase 9 expression) as cells became more non-proliferative (FIG. 14B). For iC9_CDK1 clone A, 45% of cells were viable at day 5 and 83% of cells were viable at day 18 when dosed with the highest concentration of AP20187 (“BB dimerizer”) (FIG. 14B). For iC9_CDK1 clone B, 36% of cells were viable at day 5 and 77% of cells were viable at day 18 when dosed with the highest concentration of AP20187 (FIG. 14B).

For biallelic iC9_CENPF clone A, 84% of cells were viable at day 5 and 96% of cells were viable at day 18 when dosed with the highest concentration of AP20187 (FIG. 14C). For biallelic iC9_CENPF clone B, 89% of cells were viable at day 5 and 100% of cells were viable at day 18 when dosed with the highest concentration of AP20187 (FIG. 14C).

For monoallelic iC9_TOP2A clone A, 71% of cells were viable at day 5 and 73% of cells were viable at day 18 when dosed with the highest concentration of AP20187 (FIG. 14D). For monoallelic iC9_TOP2A clone B, 72% of cells were viable at day 5 and 86% of cells were viable at day 18 when dosed with the highest concentration of AP20187 (FIG. 14D). For biallelic iC9_TOP2A clone C, 40% of cells were viable at day 5 and 56% of cells were viable at day 18 when dosed with the highest concentration of AP20187 (FIG. 14E). For biallelic iC9_TOP2A clone D, 41% of cells were viable at day 5 and 60% of cells were viable at day 18 when dosed with the highest concentration of AP20187 (FIG. 14E).

For monoallelic iC9_SMC4 clone A, 85% of cells were viable at day 5 and 87% of cells were viable at day 18 when dosed with the highest concentration of AP20187 (FIG. 14F). For monoallelic iC9_SMC4 clone B, 84% of cells were viable at day 5 and 86% of cells were viable at day 18 when dosed with the highest concentration of AP20187 (FIG. 14F).

For monoallelic iC9_AURKB clone A, 85% of cells were viable at day 5 and 95% of cells were viable at day 18 when dosed with the highest concentration of AP20187 (FIG. 14G). For monoallelic iC9_AURKB clone B, 94% of cells were viable at day 5 and 87% of cells were viable at day 18 when dosed with the highest concentration of AP20187 (FIG. 14G). For biallelic iC9_AURKB clone C, 66% of cells were viable at day 5 and 87% of cells were viable at day 18 when dosed with the highest concentration of AP20187 (FIG. 14H). For biallelic iC9_AURKB clone D, 65% of cells were viable at day 5 and 75% of cells were viable at day 18 when dosed with the highest concentration of AP20187 (FIG. 14H).

For biallelic iC9_BIRC5 clone A, 83% of cells were viable at day 5 and 91% of cells were viable at day 18 when dosed with the highest concentration of AP20187 (FIG. 14I).

For monoallelic iC9_TPX2 clone A, 66% of cells were viable at day 5 and 99% of cells were viable at day 18 when dosed with the highest concentration of AP20187(FIG. 14J). For monoallelic iC9_TPX2 clone B, 64% of cells were viable at day 5 and 93% of cells were viable at day 18 when dosed with the highest concentration of AP20187 (FIG. 14J).

For biallelic iC9_MKI67 clone A, 67% of cells were viable at day 5 and 75% of cells were viable at day 18 when dosed with the highest concentration of AP20187 (FIG. 14K). For biallelic iC9_MKI67 clone B, 58% of cells were viable at day 5 and 67% of cells were viable at day 18 when dosed with the highest concentration of AP20187 (FIG. 14K).

For monoallelic iC9_CDC20 clone A, 49% of cells were viable at day 5 and 74% of cells were viable at day 18 when dosed with the highest concentration of AP20187 (FIG. 14L). For monoallelic iC9_CDC20 clone B, 49% of cells were viable at day 5 and 79% of cells were viable at day 18 when dosed with the highest concentration of AP20187 (FIG. 14L).

For monoallelic iC9_PTTG1 clone A, 78% of cells were viable at day 5 and 74% of cells were viable at day 18 when dosed with the highest concentration of AP20187 (FIG. 14M). For monoallelic iC9_PTTG1 clone B, 78% of cells were viable at day 5 and 73% of cells were viable at day 18 when dosed with the highest concentration of AP20187 (FIG. 14M).

As shown in FIG. 14N, monoallelic iC9_KIF11 clone A, 76% of cells were viable at day 5 and 93% of cells were viable at day 18 when dosed with the highest concentration of AP20187.

For monoallelic iC9_TYMS clone A, 23% of cells were viable at day 5 and 75% of cells were viable at day 18 when dosed with the highest concentration of AP20187. (FIG. 14O).

In all the proliferation loci tested, there was progressive loss of iCaspase 9 function (decreased iCaspase 9 expression) as the cells became non-proliferative. The IC₅₀ measured 24 hours after dosing at the indicated differentiation stage for each clone is summarized in Table 14 below.

TABLE 14
IC₅₀ of iC9 KI clones.

| | Differentiation | | | |
|---|---|---|---|---|
| KI Locus | Stage | Expression | Clone | IC₅₀ (pM) |
| AAVS1 | iPSC | Monoallelic | A | 1.9 |
| AAVS1 | Day 5 | Monoallelic | A | 2.1 |
| AAVS1 | Day 18 | Monoallelic | A | 0.9 |
| AAVS1 | iPSC | Biallelic | B | 2.6 |
| AAVS1 | Day 5 | Biallelic | B | 1.5 |
| CDK1 | iPSC | Monoallelic | A | 14.1 |
| CDK1 | iPSC | Monoallelic | B | 14.8 |
| CENPF | iPSC | Biallelic | A | 68.1 |
| CENPF | iPSC | Biallelic | B | 77.5 |
| TOP2A | iPSC | Monoallelic | A | 6.7 |
| TOP2A | iPSC | Monoallelic | B | 8.4 |
| TOP2A | iPSC | Biallelic | C | 2.7 |
| TOP2A | iPSC | Biallelic | D | 2.8 |
| SMC4 | iPSC | Monoallelic | A | 42.8 |
| SMC4 | iPSC | Monoallelic | B | 43.1 |
| AURKB | iPSC | Monoallelic | A | 32 |
| AURKB | iPSC | Monoallelic | B | 35 |
| AURKB | iPSC | Biallelic | C | 12.9 |
| AURKB | iPSC | Biallelic | D | 8.8 |
| BIRC5 | iPSC | Biallelic | A | 43.4 |
| TPX2 | iPSC | Monoallelic | A | 47.4 |
| TPX2 | iPSC | Monoallelic | B | 34.7 |
| MKI67 | iPSC | Biallelic | A | 14.9 |
| MKI67 | iPSC | Biallelic | B | 19.5 |
| CDC20 | iPSC | Monoallelic | A | 7.3 |
| CDC20 | iPSC | Monoallelic | B | 8.4 |
| PTTG1 | iPSC | Monoallelic | A | 54.7 |
| PTTG1 | iPSC | Monoallelic | B | 73.1 |
| KIF11 | iPSC | Monoallelic | A | 109.8 |
| TYMS | iPSC | Monoallelic | A | 87.8 |

Top candidate proliferation loci were selected based on a low IC₅₀ and a high ratio of remaining cells after differentiation. The first criterion for candidate selection was based on efficient killing in iPSCs, which was characterized by an IC₅₀ close to 1-2 μM (IC₅₀ of AAVS1 control clones from FIG. 14A). The second criterion for selecting a proliferation locus was based on the highest percentage of cells that were resistant to kill switch activation on day 18 of spontaneous differentiation. The IC₅₀ for the kill curves at the iPSC stage plotted against the percentage of resistant cells on day 18 of candidate proliferation gene loci is shown in FIG. 15A. As shown in FIG. 15B, gene candidates (TOP2A, AURKB, CDC20, CDK1, CENPF, and TPX2) with IC₅₀ lower than 50 μM and assayed with greater than 80% viable cells in the non-proliferative state were selected for further studies.

In Vitro Validation of iCaspase 9 and Cytosine Deaminase Function and Expression in Beta-Cell Differentiation

To validate iCaspase 9 and cytosine deaminase (CDA) kill switch function in vitro, undifferentiated iPSCs and differentiated pancreatic progenitors with either iC9 or CDA knocked into the AURKB or CDK1 loci were tested for selective killing of pluripotent and proliferative cells in a directed differentiation into beta islet cells.

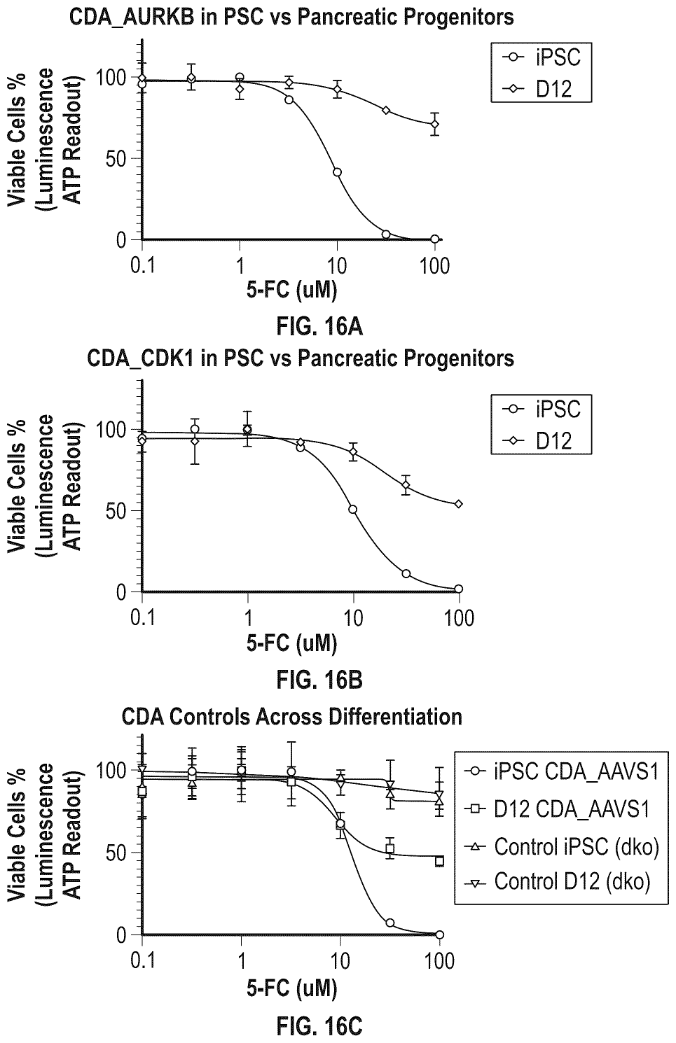

The kill switch function of CDA, which is inherently specific to proliferative cells, at the AURKB and CDK1 loci was tested in pancreatic progenitor cells generated by directed differentiation from CDA_AURKB iPSCs or CDA_CDK1 iPSCs. The control iPSC (dko) and day 12 pancreatic progenitor cells differentiated from the control iPSC (D12) maintained viability throughout increasing concentrations of 5-FC (FIG. 16C). As shown in FIG. 16A, CDA_AURKB iPSCs viability began to decrease when the cells were dosed with about 5 μM of 5-fluorocytosine (5-FC) and exhibited complete cell death when about 100 μM of 5-FC was applied. In CDA-AURKB pancreatic progenitors (pancreatic progenitors derived from CDA_AURKB iPSC), there was partial loss of CDA function at day 12 of differentiation, presumably due to the loss of AUKRB expression and the loss of proliferative potential at the pancreatic progenitor stage (FIG. 16A). When CDA was knocked into the CDK1 locus, complete cell death for CDA_CDK1 iPSCs was observed when 100 μM of 5-FC was applied, but similar to the CDA_AURKB pancreatic progenitor data (see, FIG. 16A), CDA_CDK1 pancreatic progenitors (pancreatic progenitors derived from CDA_CDK1 iPSC) also partially lost CDA function at day 12 of differentiation due to the loss of CDK1 expression and the loss of proliferative potential at the pancreatic progenitor stage (FIG. 16B). The partial loss-of-function was similarly observed in the control cell lines with CDA knocked into the AAVS1 locus. At 100 μM 5-FC, there was no viable CDA_AAVS1 iPSCs, but 50% of CDA_AAVS1 pancreatic progenitors remained viable at the same concentration of prodrug (FIG. 16C).

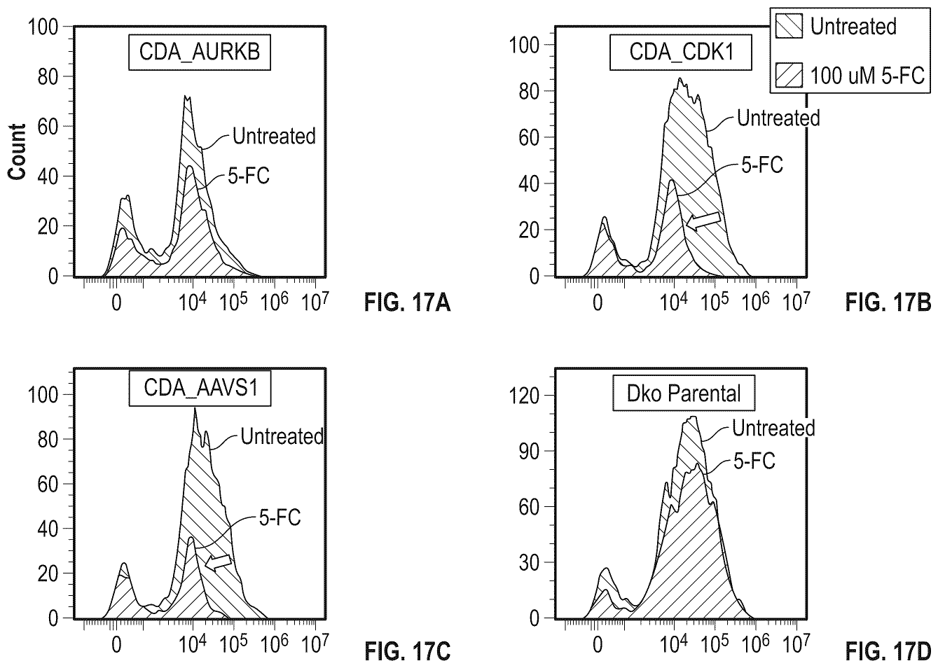

In order to test whether the CDA-induced killing was specific to proliferative cells, pancreatic progenitor cells, untreated or treated with 100 μM of 5-FC, were stained for proliferation marker Ki67 and analyzed by flow cytometry. Consistent with the cell titer assay of FIG. 16C, the control parental iPSC line (dko) showed a similar mean fluorescence intensity for Ki67 staining between untreated and 5-FC-treated cells (FIG. 17D). As shown in FIG. 17A, a large subset of Ki-67 expressing cells was cleared following activation of CDA-induced killing for CDA_AURKB pancreatic progenitors. Similar to the CDA_AAVS1 pancreatic progenitors (FIG. 17C), there was clearance of most of the proliferative cells in the CDA_CDK1 pancreatic progenitor culture (FIG. 17B).

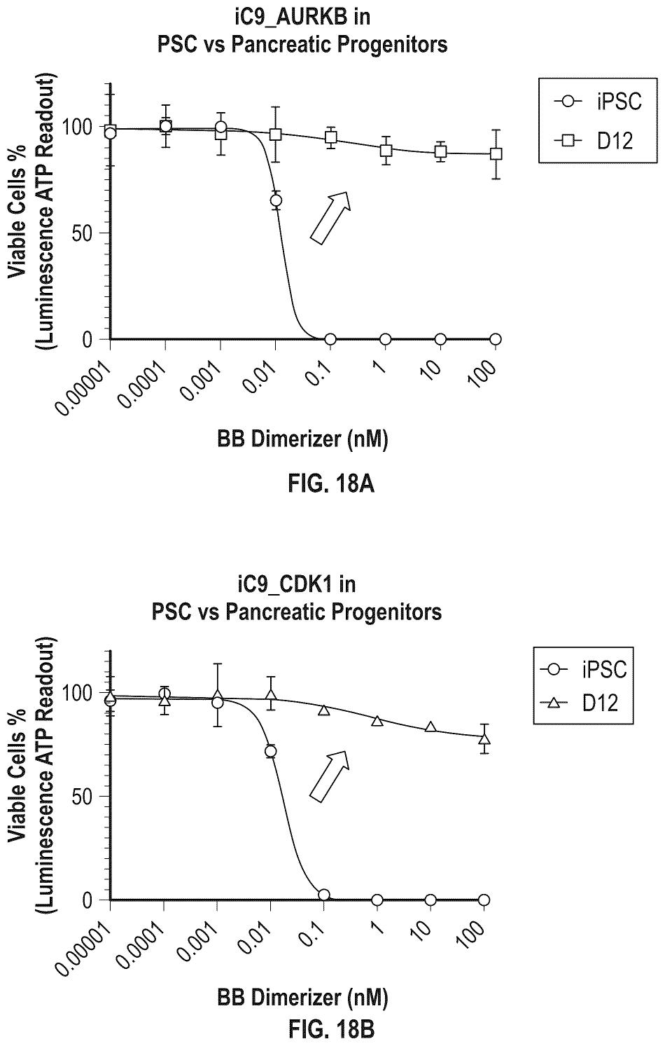

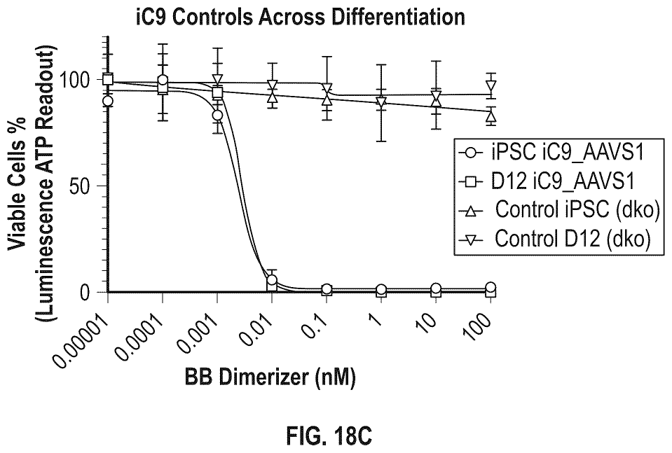

The kill switch function of iC9 at the AURKB and CDK1 loci was also tested in beta cells generated by directed differentiation. The control parental iPSC (dko) and day 12 pancreatic progenitor cells differentiated from the control iPSC remained viable throughout increasing concentrations of AP20187 (“BB dimerizer”) (FIG. 18C). Both control iC9_AAVS1 iPSC and day 12 pancreatic progenitor cells differentiated from the iC9_AAVS1 iPSC demonstrated complete killing when 0.01 nM AP20187 was added to the cultures, demonstrating no loss of iC9 function during differentiation (FIG. 18C). As seen in both iC9_AURKB (FIG. 18A) and iC9_CDK1 (FIG. 18B) iPSC cells, iCaspase 9 function effectively killed all cells when 0.1 nM AP20187 was applied. In both differentiated iC9_AURKB and iC9_CDK1 pancreatic progenitors, there was loss of iCaspase 9 function due to the loss of AURKB and CDK1 expression in pancreatic progenitors (FIGS. 18A and 18B). In vitro characterization demonstrated that safety switch function can be restricted to dividing cells without impact on PSC pluripotency, division time, and/or function.

### In Silico Screen for Gene Candidates (e.g., Off-Target Gene Candidates)

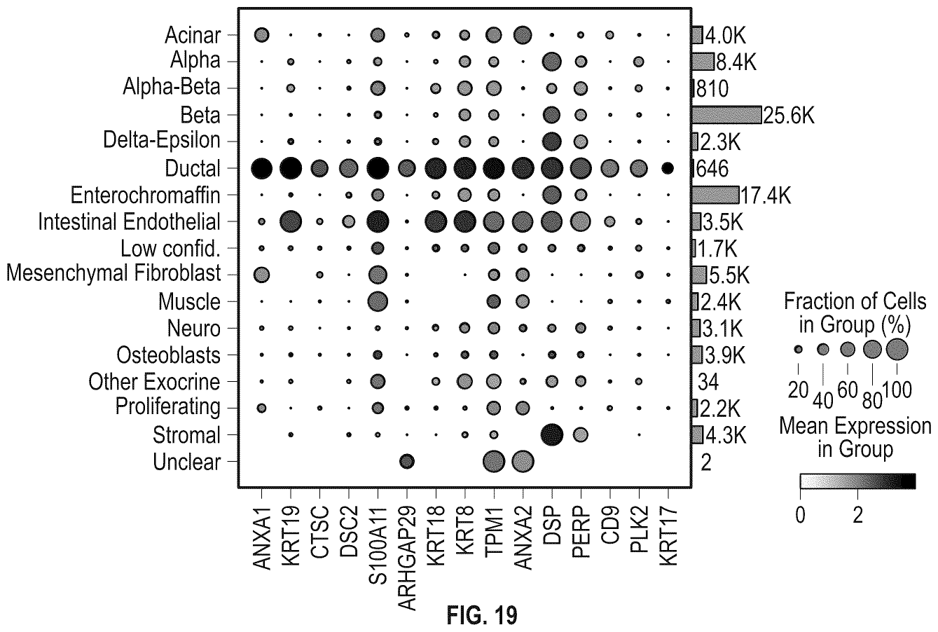

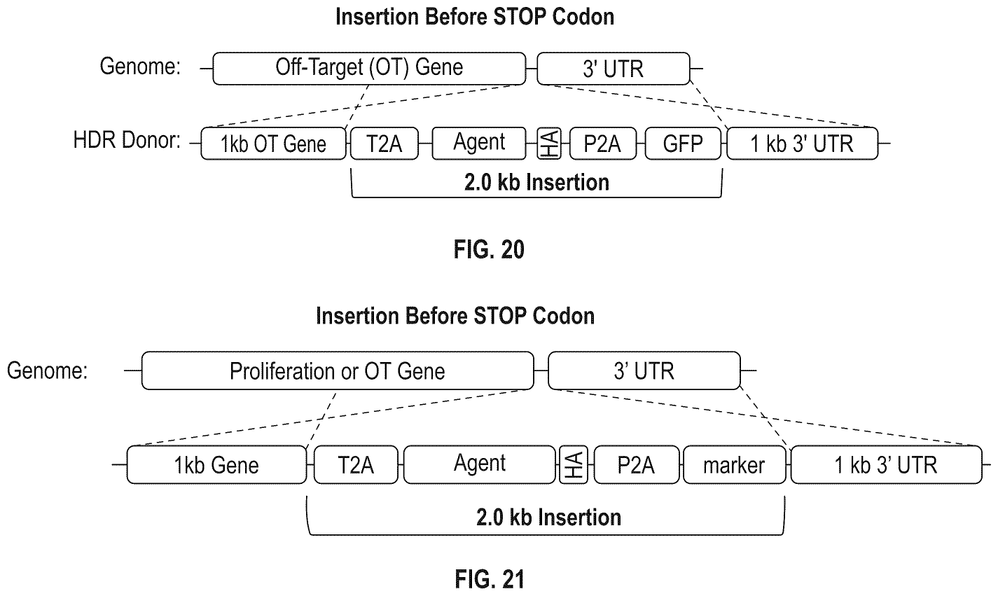

As a further proof-of-concept, markers of an off-target cell type (e.g., ductal cell markers and enterochromaffin cell markers) were identified in single-cell RNA experiments of beta-islet differentiations (n=22). These cells can be found in small numbers in beta islet differentiation batches and are potential precursors of adenocarcinomas, the most common type of pancreatic cancers. The gene markers were either specific to the subset of cells identified as ductal (e.g. KRT17, ARHGAP29) or common to more than one off-target cell type including ductal cells (e.g. ANXA1, KRT19, CTSC, DSC2, KRT18, KRT8) (FIG. 19). To knock-in a kill switch, HDR donors can introduce the kill switch into the 3′ UTR of the off-target cell marker gene locus (FIG. 20). In one embodiment, an inducible kill switch can be used to selectively kill off-target, ductal cells, made during beta islet differentiation.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A cell comprising a nucleic acid encoding a selection agent or a detection agent integrated at an endogenous proliferation gene locus and operably linked to the promoter of the endogenous proliferation gene, wherein expression of the nucleic acid encoding the selection agent or the detection agent is regulated by the promoter of the endogenous proliferation gene and/or regulatory elements of the endogenous proliferation gene.

2-6. (canceled)

7. The cell of claim 1, wherein the cell is a pluripotent cell, a progenitor cell, or a differentiated cell that is proliferating.

8-9. (canceled)

10. The cell of claim 1, wherein:
(a) the endogenous proliferation gene is expressed at a higher level from day 0 to about day 5 of differentiation, compared to expression of the endogenous proliferation gene at day 18 of differentiation, or
(b) the endogenous proliferation gene is highly expressed from day 1 to about day 5 of differentiation.

11. The cell of claim 1, wherein the endogenous proliferation gene is selected from the group consisting of AURKB, CDK1, CDC20, RRM2, BIRC5, TOP2A, PTTG1, CCNB1, TPX2, KIF11, SPC25, CENPK, SMC4, TYMS, H2AZ1, TMSB15A, CENPF, and MKI67.

12. The cell of claim 1, wherein the nucleic acid encoding the selection agent or the detection agent is integrated in (a) a 5′ UTR or a 3′ UTR of the endogenous proliferation gene, or (b) an exon or an intron of the endogenous proliferation gene.

13. (canceled)

14. The cell of claim 1, wherein following integration, the endogenous proliferation gene locus comprises i) nucleic acid encoding the endogenous proliferation gene, ii) an internal ribosomal entry site or a self-cleaving RNA site, and iii) the nucleic acid encoding the selection agent or a detection agent.

15. The cell of claim 1, wherein the selection agent or the detection agent is expressed in proliferating cells.

16-20. (canceled)

21. The cell of claim 1, wherein the cell is selected from the group consisting of a pluripotent stem cell, an induced pluripotent stem cell, a primary cell, a progenitor cell, an embryonic stem cell, a hematopoietic stem cell, a mesenchymal stem cell, an epithelial stem cell, a germline stem cell, a mammary stem cell, an olfactory adult stem cell, a hair follicle stem cell, a multipotent stem cell, an amniotic stem cell, a cord blood stem cell, a neural stem cell, a somatic stem cell, a totipotent stem cell, a fibroblast, a monocytic precursor, an exocrine cell, a pancreatic progenitor, an endocrine progenitor, a hepatoblast, a myoblast, a preadipocyte, a chondrocyte, a bone cell, a synovial cell, a tendon cell, a ligament cell, a meniscus cell, an adipose cell, a dendritic cell, a natural killer cell, a muscle cell, an erythroid-megakaryocytic cell, an eosinophil, an islet beta cell, a neuron, a cardiomyocyte, a blood cell, an exocrine progenitor, a ductal cell, an acinar cell, an alpha cell, a beta cell, a delta cell, a cholangiocyte, a brown adipocyte, a cardiac muscle cell, a PP cell, an epidermal keratinocyte, an epithelial cell, a germ cell, a skeletal joint synovium cell, a periosteum cell, a bone cell, a perichondrium cell, a pericardium cell, a meningeal cell, a keratinocyte precursor cell, a keratinocyte stem cell, a pericyte, a glial cell, an ependymal cell, a serosal cell, a heart cell, a brain cell, a spinal cord cell, a lung cell, a pancreas cell, a bladder cell, a bone marrow cell, a spleen cell, an intestine cell, a stomach cell, an islet cell, a pancreatic islet cell, an immune cell, a B cell, a T cell, a natural killer (NK) cell, a natural killer T (NKT) cell, a macrophage, an endothelial cell, a muscle cell, a smooth muscle cell, a skeletal muscle cell, a dopaminergic neuron, a retinal pigmented epithelium cell, an optic cell, a hepatocyte, a thyroid cell, a skin cell, a glial progenitor cell, a neural cell, a stem cell, an endothelial stem cell, an adipose stem cell, an adipose progenitor cell, a lung stem cell, a lung progenitor cell, a neural progenitor cell, a T cell, a CAR-T cell, a CD14+ cell, a dendritic cell, a PBMC cell, an exocrine secretory epithelial cell, a thyroid epithelial cell, a keratinizing epithelial cell, a gall bladder epithelial cell, a surface epithelial cell, a kidney cell, a primary T cell, a CD3+ T cell, a CD4+ T cell, a CD8+ T cell, a naive T cell, a regulatory T (Treg) cell, a non-regulatory T cell, a Th1 cell, a Th2 cell, a Th9 cell, a Th17 cell, a T-follicular helper (Tfh) cell, a cytotoxic T lymphocyte (CTL), an effector T (Teff) cell, a central memory T (Tcm) cell, an effector memory T (Tem) cell, a TEMRA cells, a tissue-resident memory (Trm) cell, a virtual memory T cell, an innate memory T cell, a memory stem cell (Tsc), a γδ T cell, a helper T cell, a central memory T cell, an effector memory T cell, an effector memory RA T cell, a tissue infiltrating lymphocyte, a capillary endothelial cell, a vascular endothelial cell, an aortic endothelial cell, an arterial endothelial cell, a venous endothelial cell, a renal endothelial cell, a brain endothelial cell, a liver endothelial cell, a gamma cell, an epsilon cell, hematopoietic progenitor cell, a nodal cardiomyocyte, a conducting cardiomyocyte, a working cardiomyocyte, a cardiomyocyte precursor cell, a cardiomyocyte progenitor cell, a cardiac stem cell, an atrial cardiac stem cell, a ventricular cardiac stem cell, an epicardial cell, a vascular endothelial cell, an endocardial endothelial cell, a cardiac valve interstitial cell, a cardiac pacemaker cell, a neuroectodermal cell, a neuronal cell, a neuroendocrine cell, a cholinergic cell, a serotonergic (5-HT) cell, a glutamatergic cell, a GABAergic cell, an adrenergic cell, a noradrenergic cell, a sympathetic neuronal cell, a parasympathetic neuronal cell, a sympathetic peripheral neuronal cell, an astrocyte, an oligodendrocyte, an ependymal cell, a radial glia cell, a Schwann cell, a cerebral endothelial cells (ECs), a neuronal stem cell, a neuronal progenitor cell, a white blood cell, a red blood cell, a platelet cell, a tumor cell, an enterochromaffin cell, a mesenchymal fibroblast, an osteoblast, a stromal cell, and any combination thereof.

22. A cell comprising a nucleic acid encoding a selection agent or a detection agent integrated at an off-target cell type gene locus and operably linked to the promoter of the off-target cell marker gene, wherein expression of the nucleic acid encoding the selection agent or the detection agent is regulated by the promoter of the off-target cell marker gene and/or regulatory elements of the off-target cell marker gene.

23-52. (canceled)

53. A method of eliminating proliferating cells comprising providing a population of cells comprising the cell of claim 1 with an inducer, wherein providing the inducer causes elimination of proliferating cells in the population of cells.

54-96. (canceled)

97. A method of making a cell of claim 1, the method comprising introducing a nucleic acid encoding a selection agent or a detection agent into the cell, wherein the nucleic acid encoding the selection agent or the detection agent is integrated in the genome of the cell at the endogenous proliferation gene locus.

98. A method of making a cell of claim 22, the method comprising introducing a nucleic acid encoding a selection agent or a detection agent into the cell, wherein the nucleic acid encoding the selection agent or the detection agent is integrated in the genome of the cell at the off-target cell marker gene locus.

99-103. (canceled)

104. A method of treating a patient in need thereof comprising,
(a) administering a population of cells to the patient, wherein the population of cells comprise the cell of claim 1, and
(b) activating the selection agent to eliminate one or more cells in the population of cells.

105. A method of treating a patient in need thereof comprising,
(a) administering a population of cells to the patient, wherein the population of cells comprise the cell of claim 2, and
(b) activating the selection agent to eliminate one or more cells in the population of cells.

106-140. (canceled)

141. The cell of claim 1, wherein the selection agent is a kill switch or a degron.

142. The cell of claim 141, wherein the kill switch is selected from the group consisting of an inducible caspase 9 (iCasp9), cytosine deaminase (CDA), herpes simplex virus thymidine kinase (HSV-Tk), rapamycin-activated caspase 9 (rapaCasp9), chemically regulated-SH2-delivered inhibitory tail (CRASH-IT), nitroreductase (NTR), purine nucleoside phosphorylase (PNP), horseradish peroxidase, inducible MHC-I, inducible MHC-II, CCR4, CD16, CD19, CD20, CD30, EGFR, GD2, HER1, HER2, MUC1, PSMA, and RQR8.

143-146. (canceled)

147. The cell of claim 1, wherein the detection agent is a secreted protein or a cell surface protein, and wherein the cell surface protein is selected from the group consisting of EGFR fused to a His-tag, RQRB fused to a His-tag, and CD47 fused to a His-tag.

148. (canceled)

149. The cell of claim 1, wherein the detection agent is a fluorescent protein selected from the group consisting of: green fluorescent protein (GFP), enhanced green fluorescent protein (EGFP), yellow fluorescent protein (YFP), enhanced yellow fluorescent protein (EYFP), blue fluorescent protein (BFP), enhanced blue fluorescent protein (EBFP), cyan fluorescent protein (CFP), enhanced cyan fluorescent protein (ECFP), superfolder GFP, superfolder YFP, orange fluorescent protein, red fluorescent protein, small ultrared fluorescent protein, FMN-binding fluorescent protein, dsRed, gFP611, Dronpa, TagRFP, KFP, EosFP, IrisFP, Dendra, Kaede, KikGrl, emerald fluorescent protein, Azami Green, mWasabi, TagGFP, TurboGFP, AcGFP, ZsGreen, and T-Sapphire.

150-160. (canceled)

161. The cell of claim 1, wherein the cell is a human cell.

162-165. (canceled)

166. The cell of claim 1, wherein the cell is a stem cell-derived beta islet cell.

167-196. (canceled)


SEQUENCE LISTING (347 sequences)

SEQ ID NO: 1 - protein, 305 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 2 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 3 - protein, 85 aa, synthetic construct
WTESLYCGVYTNAWPSSDFRFEYLSSSTITWTSLYEICGVQRDTYSQLKGKKRQASNQKTIQPPRKAVEEPLNAFKESKGMMNDE

SEQ ID NO: 4 - protein, 108 aa, synthetic construct
KNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNE

SEQ ID NO: 5 - protein, 183 aa, synthetic construct
MEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVF

SEQ ID NO: 6 - DNA, 918 bp, synthetic construct
  source 1..918: mol_type = other DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactga

SEQ ID NO: 7 - DNA, 972 bp, synthetic construct
  source 1..972: mol_type = other DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataa

SEQ ID NO: 8 - DNA, 258 bp, synthetic construct
  source 1..258: mol_type = other DNA
tggactgagtctctgtattgcggcgtgtataccaatgcatggccctcttctgatttcaggtttgagtatcttagctctagcacaattacttggactagtttatatgaaatttgtggggttcagagggacacctacagtcagttgaaaggcaagaagagacaagcttccaatcagaagactatacaacctcctaggaaagctgtagaggaaccccttaatgcattcaaagaatcaaaaggaatgatgaatgatgaataa

SEQ ID NO: 9 - DNA, 6796 bp, synthetic construct
  source 1..6796: mol_type = other DNA
agatattttcttgttcaatttaaggagaggtaaatttggtatcaatagaaaaaatgtttctgaaaaatttaaaccctggaaatgtatttatggcatggagtcagatgtttcagggagagaagaacaaatcaagaagcattgcaagtatgctcatatggaatgcttaaggcttgtggttaaaaaatatatatatatggctgtcaatgtcttaggctcatggtagcagcagaaatcgtaataattcttttgtcacatgggttatatccatattggagagaattaactcaggtgaaattaacttgtacactgtttggttttataatatttagagggatcacaactgactgatgtccctttgaagtaccattcttcataaatctttttttttcagaatgggccagccaactgtgacatcccttggatcggagatttagaactagaaagtattctttctacattattagggaagaaaaggagttacttggcggttagcaatattctattttgttttgttttgtttttagagacagggtctcattatgttgaccaggctggcctcgagctcctgggctcaagcaatgctcccacctcagcctcccaagtagctgggactacaggcatgtgccactacacctggcagtgtttattctgataaatacatttatgagctcaaaaatgtaactctaaaaccttatctctgaacttccatattaccatcagaaatttagatagttgtttagttctctttttctttgtagaacatagatataaggcatggtttcattgaagtcagttgtatatacatgtaactatcctgatgttcccaaataaagctctgtatttctgcttagtttattggggaggctgctaaatgtagtgcatcccaacccattttaccctgttctactttaaaaagaggttggcttcttgtttggatacaaggaccaagtcactcccccaggttcctccacagtaagggaggcctatttaaagccgcccatggcactaacagaaactggactcctatgagctcagatacataactgggcctcacaggggtgggacagtatgtagtctaggaattggaaggatccattccatatcaaagaactgaagcatcgtgttgccctctcagcagcaagagtaaggtgatgcccctgtcagttatagttcctgagttcctctgtctttgattctttgcctattagccagctagctcaccctcttgtttatgccactgttttttatcctattcatgccttctcacagacaacttttcttacctacagctttggactcatccttgtctcctttctgtttctttttcactttcccttcccatcaccaactttctgggtttttttctgtttcttcttagagtccagtggcagggagaaacttgtcagtccagtctgttgccatttttcctgtttgagaaagactcaccagcttttggctggctcacagattggctttccttgggtcaggacccacccttttccctgccagctttggaagcttgacagaattcgagtgtgcagtggtggtaaataaatagtaaggaacacagagcagtcctggaggcgtgcctccatctgctgatgagaaaatccagtgctgtcatccagcccaggtcccagcggaatgggcctctctgttcagtaggatccccctcctgctgagtggttcatggcatgtttctgttcaacgcttttccatctgtaggattcttattctgtatttatttgtttttttgggtttttttattttttgagatggagtctcgctctgtcgcccaggctggagtgcagtggcacgaccccagctcgctgcagcctctgcctcccaggacgagggagatcctcccacctcagccttccacgtagctgggactacaggcatgcaccacaggcatgcaccaccacgccagctaatttttgtatttttggtagagacagggttgcatcatgttgcccaggctggtcttgaatgcctgagctcaagcaatctatttgccttggcctcccaaagtgctgggattacaggcatgagccaccacggccagccttctcatttgttttttttataaggaagctatctcttcttccctccccaactagggtattctttttccctttcgtcactttgctcatgtactgtattccttcaacttcattaatgaatccatttggaagcagtgaaaaaggcaactcagaaagctaagaagaaatagatagaggaatactcagagctatctgagtattttctttagtttgttagctctttggagctttgaaactggaaagacccagggagtgatgtggagaaagagactgagcttgtaagacacaggagcagtgagctaagggagatggagtagtggggacaaattctggcacattctgtctacactctgggtagatagaggagggaggatggagcacccatggtgggggtatgttggtgacagcattttcccaccagccagtgtaacaagtggctgatttgggggaaagatggcataaacaaatgagagaatgtgtttactatttgatgtagatgggttatttgcttcatttttcaaatcagtgtatataatcaagaatattcagcatgtttgaatagactgtcagagctggaactctttcattaacatctctggcacctttagttttagccctgaacattttatcttaaaattaaacattaccaaatgccttagtttatttcatttattaaatttatattcttatttgttatttatatcagcttccaatcagaagactatacaacctcctaggaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctat

SEQ ID NO: 10 - DNA, 327 bp, synthetic construct
  source 1..327: mol_type = other DNA
aagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgaataa

SEQ ID NO: 11 - DNA, 549 bp, synthetic construct
  source 1..549: mol_type = other DNA
atggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgttt

SEQ ID NO: 12-252 - skipped (241 entries)

SEQ ID NO: 253 - protein, 41 aa, synthetic construct
RSKRSRLLHSDYMNMTPRRPGPTRKHYQPYAPPRDFAAYRS

SEQ ID NO: 254 - protein, 42 aa, synthetic construct
KRGRKKLLYIFKQPFMRPVQTTQEEDGCSCRFPEEEEGGCEL

SEQ ID NO: 255 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 256 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 257 - protein, 12 aa, synthetic construct
ESKYGPPCPPCP

SEQ ID NO: 258 - protein, 25 aa, synthetic construct
TTTPAPRPPTPAPTIASQPLSLRPE

SEQ ID NO: 259 - protein, 39 aa, synthetic construct
IEVMYPPPYLDNEKSNGTIIHVKGKHLCPSPLFPGPSKP

SEQ ID NO: 260 - protein, 44 aa, synthetic construct
ACRPAAGGAVHTRGLDFACDIYIWAPLAGTCGVLLLSLVITLYC

SEQ ID NO: 261 - protein, 27 aa, synthetic construct
FWVLVVVGGVLACYSLLVTVAFIIFWV

SEQ ID NO: 262 - protein, 41 aa, synthetic construct; identical to SEQ ID NO: 253

SEQ ID NO: 263 - protein, 42 aa, synthetic construct; identical to SEQ ID NO: 254

SEQ ID NO: 264 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 265 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 266 - protein, 21 aa, synthetic construct
MALPVTALLLPLALLLHAARP

SEQ ID NO: 267 - protein, 20 aa, synthetic construct
METDTLLLWVLLLWVPGSTG

SEQ ID NO: 268 - protein, 22 aa, synthetic construct
MLLLVTSLLLCELPHPAFLLIP

SEQ ID NO: 269 - protein, 45 aa, synthetic construct
TTTPAPRPPTPAPTIASQPLSLRPEACRPAAGGAVHTRGLDFACD

SEQ ID NO: 270 - protein, 42 aa, synthetic construct
AAAIEVMYPPPYLDNEKSNGTIIHVKGKHLCPSPLFPGPSKP

SEQ ID NO: 271 - protein, 12 aa, synthetic construct
ESKYGPPCPSCP

SEQ ID NO: 272 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 273 - protein, 24 aa, synthetic construct
IYIWAPLAGTCGVLLLSLVITLYC

SEQ ID NO: 274 - protein, 28 aa, synthetic construct
MFWVLVVVGGVLACYSLLVTVAFIIFWV

SEQ ID NO: 275 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 276 - protein, 6 aa, synthetic construct
QDISKY

SEQ ID NO: 277 - skipped

SEQ ID NO: 278 - protein, 9 aa, synthetic construct
QQGNTLPYT

SEQ ID NO: 279 - protein, 18 aa, synthetic construct
GSTSGSGKPGSGEGSTKG

SEQ ID NO: 280 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 281 - protein, 8 aa, synthetic construct
GVSLPDYG

SEQ ID NO: 282 - protein, 7 aa, synthetic construct
IWGSETT

SEQ ID NO: 283 - protein, 14 aa, synthetic construct
AKHYYYGGSYAMDY

SEQ ID NO: 284 - protein, 15 aa, synthetic construct
GGGGSGGGGSGGGGS

SEQ ID NO: 285 - DNA, 1467 bp, synthetic construct
  source 1..1467: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 286 - protein, 489 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 287-292 - skipped (6 entries)

SEQ ID NO: 293 - protein, 246 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 294 - protein, 106 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 295 - protein, 10 aa, synthetic construct
RASSSVNYMD

SEQ ID NO: 296 - protein, 7 aa, synthetic construct
ATSNLAS

SEQ ID NO: 297 - protein, 9 aa, synthetic construct
QQWSFNPPT

SEQ ID NO: 298 - protein, 122 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 299 - protein, 5 aa, synthetic construct
SYNMH

SEQ ID NO: 300 - protein, 17 aa, synthetic construct
AIYPGNGDTSYNQKFKG

SEQ ID NO: 301 - protein, 13 aa, synthetic construct
SNYYGSSYWFFDV

SEQ ID NO: 302 - protein, 246 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 303 - protein, 124 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 304 - protein, 10 aa, synthetic construct
GDSVSSNSAA

SEQ ID NO: 305 - protein, 9 aa, synthetic construct
TYYRSKWYN

SEQ ID NO: 306 - protein, 14 aa, synthetic construct
AREVTGDLEDAFDI

SEQ ID NO: 307 - protein, 107 aa, synthetic construct
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 308 - protein, 6 aa, synthetic construct
QTIWSY

SEQ ID NO: 309 - skipped

SEQ ID NO: 310 - protein, 9 aa, synthetic construct
QQSYSIPQT

SEQ ID NO: 311 - protein, 246 aa, synthetic construct
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 312 - protein, 124 aa, synthetic construct
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSS

SEQ ID NO: 313 - protein, 10 aa, synthetic construct
GDSVSSNSVA

SEQ ID NO: 314 - protein, 9 aa, synthetic construct
TYYRSTWYN

SEQ ID NO: 315 - protein, 107 aa, synthetic construct
DIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 316 - protein, 246 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 317 - protein, 111 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 318 - protein, 15 aa, synthetic construct
RASESVSVIGAHLIH

SEQ ID NO: 319 - protein, 7 aa, synthetic construct
LASNLET

SEQ ID NO: 320 - protein, 9 aa, synthetic construct
LQSRIFPRT

SEQ ID NO: 321 - protein, 117 aa, synthetic construct
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 322 - protein, 5 aa, synthetic construct
DYSIN

SEQ ID NO: 323 - protein, 17 aa, synthetic construct
WINTETREPAYAYDFRG

SEQ ID NO: 324 - protein, 8 aa, synthetic construct
DYSYAMDY

SEQ ID NO: 325 - protein, 246 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 326 - protein, 111 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 327 - protein, 15 aa, synthetic construct
RASESVTILGSHLIY

SEQ ID NO: 328 - protein, 7 aa, synthetic construct
LASNVQT

SEQ ID NO: 329 - protein, 9 aa, synthetic construct
LQSRTIPRT

SEQ ID NO: 330 - protein, 117 aa, synthetic construct
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 331 - protein, 5 aa, synthetic construct
HYSMN

SEQ ID NO: 332 - protein, 17 aa, synthetic construct
RINTESGVPIYADDFKG

SEQ ID NO: 333 - protein, 8 aa, synthetic construct
DYLYSLDF

SEQ ID NO: 334 - protein, 121 aa, synthetic construct
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 335 - protein, 8 aa, synthetic construct
GFTFSSYA

SEQ ID NO: 336 - protein, 8 aa, synthetic construct
ISGSGDYI

SEQ ID NO: 337 - protein, 14 aa, synthetic construct
AKEGTGANSSLADY

SEQ ID NO: 338 - protein, 243 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 339 - protein, 106 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 340 - protein, 6 aa, synthetic construct
QSISSY

SEQ ID NO: 341 - protein, 8 aa, synthetic construct
QQKYDLLT

SEQ ID NO: 342 - protein, 119 aa, synthetic construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 343 - protein, 10 aa, synthetic construct
GGSISSSSYY

SEQ ID NO: 344 - protein, 7 aa, synthetic construct
ISYSGST

SEQ ID NO: 345 - protein, 11 aa, synthetic construct
ARDRGDTILDV

SEQ ID NO: 346 - DNA, 1503 bp, synthetic construct
  source 1..1503: mol_type = other DNA
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaaaatacgacctcctcacttttggcggagggaccaaggttgagatcaaaggcagcaccagcggctccggcaagcctggctctggcgagggcagcacaaagggacagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagagatcgtggagacaccatactagacgtatggggtcagggtacaatggtcaccgtcagctcattcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 347 - protein, 501 aa, synthetic construct
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR